(19) United States
(12) Patent Application Publication
(10) Pub. No.: US 2023/0395193 A1
(43) Pub. Date: Dec. 7, 2023

(54) SYSTEMS FOR AND METHODS OF TREATMENT SELECTION

(71) Applicant: THE REGENTS OF THE UNIVERSITY OF CALIFORNIA, Oakland, CA (US)

(72) Inventors: Nevan J. KROGAN, San Francisco, CA (US); Kliment VERBA, San Francisco, CA (US)

(21) Appl. No.: 18/032,163

(22) PCT Filed: Oct. 14, 2021
(86) PCT No.: PCT/US2021/055096

§ 371 Date: Apr. 14, 2023

Related U.S. Application Data
(60) Provisional application No. 63/091,929, filed on Oct. 15, 2020.

(51) Int. Cl. G16B 20/40; G16B 15/00; G01N 33/68; G16H 50/70; G16H 50/20; G16H 20/10

(52) U.S. Cl. CPC G16B 20/40; G16B 15/00; G01N 33/6848; G16H 50/70; G16H 50/20; G16H 20/10

(57) ABSTRACT

The disclosure relates to a system comprising software that predicts responsiveness of subjects to certain disease modifying drugs. Embodiments of the disclosure include methods comprising calculating a differential interaction score (DIS), correlating the DIS with the likelihood that a dysfunctional protein-protein interaction is the causal agent of a disease or disorder, and identifying a subject responsive to a treatment based upon the causal agent.

### CROSS-REFERENCE TO RELATED APPLICATIONS

This application claims the benefit of U.S. Application No. 63/091,929, filed on Oct. 15, 2020, the contents of which are hereby incorporated by reference in their entirety.

### STATEMENT REGARDING FEDERALLY SPONSORED RESEARCH

This invention was made with government support under grants P01 AI063302, P50 AI150476, R01 AI143292, U19 AI135972, and U19 AI135990 awarded by The National Institutes of Health, and grant HR001-11-9-2002 awarded by The Defense Advanced Research Projects Agency. The government has certain rights in the invention.

### FIELD OF INVENTION

The disclosure relates to a system comprising software that identifies drug targets and predicts responsiveness of subjects to certain disease modifying drugs. Embodiments of the disclosure include methods comprising calculating a differential interaction score (DIS), correlating the DIS with the likelihood that a dysfunctional protein-protein interaction is the causal agent of a disorder, such as, for example, viral and bacterial diseases, cancers, neurodegenerative diseases, and neuropsychiatric disorders, identifying a drug target based on the causal agent, evaluating a therapeutic specific to the drug target, thereby restoring and/or alleviating dysfunction within the protein network, identifying a subject responsive to a treatment based upon the causal agent, and monitoring the subject's response to the treatment.

### BACKGROUND

In the past two decades, three new deadly human respiratory syndromes associated with coronavirus (CoV) infections emerged: Severe Acute Respiratory Syndrome (SARS) in 2002, Middle East Respiratory Syndrome (MERS) in 2012, and Coronavirus Disease 2019 (COVID-19) in 2019. These three diseases are caused by the zoonotic CoVs SARS-CoV-1, MERS-CoV, and SARS-CoV-2 (A comparative overview of COVID-19, MERS and SARS: Review article. Int. J. Surg. 81), respectively. Before their emergence, human CoVs were associated with usually mild respiratory illness. To date, SARS-CoV-2 has sickened millions and killed almost one million worldwide. This unprecedented challenge has prompted widespread efforts to develop new vaccine and antiviral strategies, including repurposed therapeutics, which offer the potential for treatments with known safety profiles and short development timelines. The successful repurposing of the antiviral nucleoside analog Remdesivir (Beigel, et al., Remdesivir for the treatment of Covid-19—preliminary report. N. Engl. J. Med. (2020)), as well as the host-directed anti-inflammatory steroid dexamethasone (T. R. C. Group, The RECOVERY Collaborative Group, Dexamethasone in Hospitalized Patients with Covid-19—Preliminary Report. New England Journal of Medicine (2020)), provide clear proof that existing compounds can be crucial tools in the fight against COVID-19. Despite these promising examples, there is still no curative treatment for COVID-19. In addition, as with any virus, the search for effective antiviral strategies could be complicated over time by the continued evolution of SARS-CoV-2 and possible resulting drug resistance (M. Becerra-Flores, T. Cardozo, SARS-CoV-2 viral spike G614 mutation exhibits higher case fatality rate. Int. J. Clin. Pract. (2020), doi:10.1111/ijcp.13525).

Current endeavors are appropriately focused on SARS-CoV-2 due to the severity and urgency of the ongoing pandemic. However, the frequency with which other highly virulent CoV strains have emerged highlights an additional need to identify promising targets for broad CoV inhibitors with high barriers to resistance mutations and potential for rapid deployment against future emerging strains. While traditional antivirals target viral enzymes that are often subject to mutation and thus the development of drug resistance, targeting the host proteins required for viral replication is a strategy that can avoid resistance and lead to therapeutics with the potential for broad-spectrum activity as families of viruses often exploit common cellular pathways and processes.

Accordingly, there remains a need for methods and systems for facilitating interpretation of viral biology, in general, and, more specifically, of coronavirus biology, predicting clinical outcomes, and developing treatment strategies.

### SUMMARY OF EMBODIMENTS

Here, shared biology and potential drug targets are identified among the three highly pathogenic human CoV strains. The recently published map of virus-host protein interactions for SARS-CoV-2 was expanded on (Gordon, et al. A SARS-CoV-2 protein interaction map reveals targets for drug repurposing. Nature (2020)), and mapped the full interactome of SARS-CoV-1 and MERS-CoV. The localization of viral proteins across strains was investigated, and the virus-human interactions for each virus was quantitatively compared. Using functional genetics and structural analysis of selected host-dependency factors, drug targets were identified, and real-world analysis performed on clinical data from COVID-19 patient outcomes.

The present disclosure therefore relates to methods of identifying a therapeutic target for a disorder treatment, the method comprising: (a) compiling genetic data about a population of subjects that has a mutation candidate that causes a disorder; (b) performing a mass spectrometry analysis on a sample associated with the disorder to identify dysfunctional protein-protein interactions associated with the disorder; (c) calculating a differential interaction score (DIS); (d) correlating the DIS with the likelihood that the dysfunctional protein-protein interaction is a causal agent of the disorder, wherein if the DIS score is above a first threshold, then the causal agent is selected as a therapeutic target for the disorder treatment, and wherein if the DIS score is below the first threshold, then the causal agent is not selected as a therapeutic target for the disorder treatment.

The disclosure further relates to methods of identifying a therapeutic target for a hyperproliferative disorder treatment, the method comprising: (a) calculating a differential interaction score (DIS); and (b) correlating the DIS with a likelihood that a dysfunctional protein-protein interaction is a causal agent of the disorder, wherein if the DIS score is above a first threshold, then the causal agent is selected as a therapeutic target for the disorder treatment, and wherein if the DIS score is below the first threshold, then the causal agent is not selected as a therapeutic target for the disorder treatment.

The disclosure further relates to methods of identifying a therapeutic for treating a disorder, the method comprising screening a candidate compound for binding with, or activity against a therapeutic target, wherein the therapeutic target was identified via a disclosed method.

The disclosure further relates to methods of predicting a likelihood that a disorder is responsive to a therapeutic, the method comprising: (a) compiling genetic data about a population of subjects that has a mutation candidate that causes a disorder; (b) performing a mass spectrometry analysis on a sample associated with the disorder to identify dysfunctional protein-protein interactions associated with the disorder; (c) calculating a differential interaction score (DIS); (d) correlating the DIS with the likelihood that the dysfunctional protein-protein interaction is the causal agent of the disorder; and (e) selecting a therapeutic for treating the disorder based upon the causal agent.

The disclosure further relates to methods of identifying an interaction between a pathogen protein and a host protein, the method comprising: (a) identifying a first pathogen protein that co-localizes with a first host protein in one or a plurality of bioassays; (b) calculating a differential interaction score (DIS) corresponding to a pathogen protein and a host protein in a sample; and (c) correlating the DIS with the likelihood that the dysfunctional protein-protein interaction is a causal agent of pathogenicity of the pathogen.

The disclosure further relates to methods of identifying an interaction between a first protein and a second protein, wherein the first protein is associated with a disorder of a subject, the method comprising: (a) identifying a first protein that co-localizes with the second protein in one or a plurality of bioassays; (b) calculating a differential interaction score (DIS) corresponding to the first protein and a second protein in a sample from the subject; and (c) correlating the DIS with the likelihood that the dysfunctional protein-protein interaction is a causal agent of pathogenicity of the pathogen.

The disclosure further relates to methods of identifying a subject likely to respond to a disorder treatment, the method comprising: (a) calculating a differential interaction score (DIS); and (b) correlating the DIS with a likelihood that a dysfunctional protein-protein interaction is a causal agent of the disorder, wherein if the DIS score is above a first threshold, then the subject is likely to respond to a disorder treatment based upon the causal agent, and wherein if the DIS score is below the first threshold, then the subject is not likely to respond to the disorder treatment based upon the causal agent.

The disclosure further relates to methods of predicting a likelihood that a subject does or does not respond to a disorder treatment, the method comprising: (a) compiling genetic data about a population of subjects that has a mutation candidate that causes a disorder, wherein the population of subjects includes the subject; (b) performing a mass spectrometry analysis on a sample associated with the disorder to identify dysfunctional protein-protein interactions associated with the disorder; (c) calculating a differential interaction score (DIS); (d) correlating the DIS with the likelihood that the dysfunctional protein-protein interaction is the causal agent of the disorder; and (e) selecting a treatment for the subject based upon the causal agent.

The disclosure further relates to computer program products encoded on a computer-readable storage medium, wherein the computer program product comprises instructions for: (a) identifying protein-protein interactions associated with the disorder; and (b) calculating a differential interaction score (DIS).

The disclosure further relates to systems for identifying a protein interaction network in a subject, the system comprising: (a) a processor operable to execute programs; (b) a memory associated with the processor; (c) a database associated with said processor and said memory; and (d) a program stored in the memory and executable by the processor, the program being operable for: (i) performing a mass spectrometry analysis on a sample from a subject that has a mutation candidate that causes a disorder; (ii) identifying dysfunctional protein-protein interactions associated with the disorder; and (iii) calculating a differential interaction score (DIS).

The disclosure further relates to methods of treating a viral infection due to a Coronavirus in a subject having a genetic alteration in PGES-2 signaling, the method comprising administering to the subject a pharmaceutically effective amount of a PGES-2 inhibitor, wherein the subject was previously identified as being in need of treatment by: (a) performing a mass spectrometry analysis on a sample from the subject; (b) identifying dysfunctional protein-protein interactions associated with the viral infection; and (c) calculating a differential interaction score (DIS).

The disclosure further relates to methods of treating a Coronaviridae viral infection in a subject in need thereof, the method comprising administering to the subject a pharmaceutically effective amount of a sigma receptor inhibitor, wherein the subject was previously identified as being in need of treatment by: (a) performing a mass spectrometry analysis on a sample from the subject; (b) identifying dysfunctional protein-protein interactions associated with the viral infection; and (c) calculating a differential interaction score (DIS).

The disclosure further relates to methods of selecting a disorder treatment for a subject in need thereof, the method comprising: (a) identifying genetic data from the subject in need of treatment; (b) comparing the genetic data from the subject to a compilation of genetic data from population of subjects that has a mutation candidate that causes a disorder, wherein the population of subjects includes the subject in need thereof; (c) performing a mass spectrometry analysis on a sample from the subject associated with the disorder to identify dysfunctional protein-protein interactions associated with the disorder; (d) calculating a differential interaction score (DIS); (e) correlating the DIS with the likelihood that the dysfunctional protein-protein interaction is a causal agent of the disorder; and (f) selecting a disorder treatment for the subject based upon the causal agent.

Still other objects and advantages of the present disclosure will become readily apparent by those skilled in the art from the following detailed description, wherein it is shown and described only the preferred embodiments, simply by way of illustration of the best mode. As will be realized, the disclosure is capable of other and different embodiments, and its several details are capable of modifications in various obvious respects, without departing from the disclosure. Accordingly, the description is to be regarded as illustrative in nature and not as restrictive.

### BRIEF DESCRIPTION OF THE DRAWINGS

The accompanying figures, which are incorporated in and constitute a part of this specification, illustrate several embodiments and together with the description serve to explain the principles of the invention.

FIG. 1A-E show representative data illustrating an overview of coronavirus genome annotations and integrative analysis. Specifically, FIG. 1A shows the genome annotation of SARS-CoV-2, SARS-CoV-1, and MERS-CoV with putative protein coding genes highlighted. The intensity of the filled color indicates the lowest sequence identity between SARS-CoV2 and SARS-CoV-1 or SARS-CoV-2 and MERS. FIG. 1B-D show the genome annotation of structural protein genes for SARS-CoV-2 (FIG. 1B), SARS-CoV-1 (FIG. 1C), and MERS-CoV (FIG. 1D). Color intensity indicates sequence identity to specified virus. FIG. 1E shows an overview of comparative coronavirus analysis. Proteins from SARS-CoV-2, SARS-CoV-1, and MERS-CoV were analyzed for their protein interactions and subcellular localization, and these data were integrated for comparative host interaction network analysis, followed by functional, structural, and clinical data analysis for exemplary virus-specific and pan-viral interactions. The SARS-CoV-2 interactome was previously published in a separate study (D. E. Gordon, Nature (2020)). SARS=both SARS-CoV-1 and SARS-CoV-2; MERS=MERS-CoV; Nsp=non-structural protein; Orf=open reading frame.

FIG. 2A-G show representative data illustrating a comparative analysis of coronavirus-host interactomes.

FIG. 3A-F show representative viabilites, knockdown efficiencies, and editing efficiencies in response to siRNA and CRISPR perturbations.

FIG. 4A-F show representative data illustrating the functional interrogation of SARS-CoV-2 interactors using genetic perturbations.

FIG. 5A-C show representative data illustrating the predicted binding modes of mPGES-2 and Nsp7.

FIG. 6A-F show a representative analysis of coronavirus protein localization.

FIG. 7 shows representative data illustrating the immunolocalization of Strep-tagged SARS-CoV-2 non-structural (Nsp) proteins. HeLaM cells were transfected with 2×Strep-tagged viral proteins, fixed, and immunostained with anti-Strep antibodies. Samples were also immunostained for the Golgi-localized protein Syntaxin 5 (STX5). Scale bar=10 μm.

FIG. 8 shows representative data illustrating the immunolocalization of Strep-tagged SARS-CoV-2 structural and accessory proteins. HeLaM cells were transfected with 2×Strep-tagged viral proteins, fixed, and immunostained with anti-Strep antibodies. Samples were also immunostained for the Golgilocalized protein Syntaxin 5 (STX5). Scale bar=10 μm.

FIG. 9 shows representative data illustrating the immunolocalization of Strep-tagged SARS-CoV-1 non-structural (Nsp) proteins. HeLaM cells were transfected with 2×Strep-tagged viral proteins, fixed, and immunostained with anti-Strep antibodies. Samples were also immunostained for the Golgilocalized protein Syntaxin 5 (STX5). Scale bar=10 μm.

FIG. 10 shows representative data illustrating the immunolocalization of Strep-tagged SARS-CoV-1 structural and accessory proteins. HeLaM cells were transfected with 2×Strep-tagged viral proteins, fixed, and immunostained with anti-Strep antibodies. Samples were also immunostained for the Golgilocalized protein Syntaxin 5 (STX5). Scale bar=10 μm. Ring structures formed by SARS-CoV1 Orf6 highlighted in enlarged micrograph image.

FIG. 11 shows representative data illustrating the immunolocalization of Strep-tagged MERS-CoV non-structural (Nsp) proteins. HeLaM cells were transfected with 2×Strep-tagged viral proteins, fixed, and immunostained with anti-Strep antibodies. Samples were also immunostained for the Golgi-localized protein Syntaxin 5 (STX5). Scale bar=10 μm.

FIG. 12 shows representative data illustrating the immunolocalization of Strep-tagged MERS-CoV structural and accessory proteins. HeLaM cells were transfected with 2×Strep-tagged viral proteins, fixed, and immunostained with anti-Strep antibodies. Samples were also immunostained for the Golgilocalized protein Syntaxin 5 (STX5). Scale bar=10 μm. Ring structures formed by MERS-CoV Orf8b highlighted in enlarged micrograph image.

FIG. 13 shows representative data illustrating the immunolocalization of SARS-CoV-2 proteins in infected Caco-2 cells. Caco-2 cells were infected with SARS-CoV-2, fixed, and immunostained with specific polyclonal antibodies. Samples were co-stained with anti-PDI or Alexa Fluor 647-conjugated phalloidin, and nuclei were stained with DAPI. Scale bar=10 μm.

FIG. 14A-D show representative data illustrating a comparison of enriched terms and shared interactors across viruses.

FIG. 15A-D show representative data illustrating that a comparative differential interaction analysis reveals shared virus-host interactions.

FIG. 16A-G show representative data illustrating the interaction between Orf9b and human Tom70.

FIG. 17A-C show representative data illustrating that Org9b interacts specifically with Tom70.

FIG. 18A-E show representative data illustrating that the CryoEM structure of Orf9b-Tom70 complex reveals Orf9b adopting a helical fold and binding at the substrate recognition site of Tom70.

FIG. 19A-C show representative data illustrating an Orf9b-Tom70 cryoEM density map and the Fourier Shell Correlation of the final reconstruction.

FIG. 20 shows a representative image illustrating subtle conformational changes at the MEEVD binding site of Tom70.

FIG. 21A-F show representative data illustrating that SARS-CoV-2 Orf8 and functional interactor IL17RA are linked to viral outcomes.

FIG. 22A-E show representative data illustrating the perturbation of drug targets and the performance of selected drugs against coronavirus replication in vitro.

FIG. 23A-D show representative data illustrating that real-world data analysis of drugs identified through molecular investigation support their antiviral activity.

FIG. 24 shows representative data illustrating departures from neutral evolution in SIGMAR1.

FIG. 25 shows representative images illustrating SARS-CoV-1 protein expression. Input samples from immunoprecipitations were probed by immunoblot using anti-Strep antibody. Red arrowhead indicates that the band appears near expected molecular weight. Nsp=non-structural protein; Orf=open reading frame.

FIG. 26 shows representative images illustrating MERS-CoV protein expression. Input samples from immunoprecipitations were probed by immunoblot using anti-Strep antibody. Red arrowhead indicates that the band appears near expected molecular weight. Nsp=non-structural protein; Orf=open reading frame.

FIG. 27 shows representative data illustrating a correlation analysis of SARS-CoV-1 proteomics samples. Pearson's pairwise correlations were calculated for all combinations of replicates of SARS-CoV-1 affinity purification-mass spectrometry (AP-MS) samples. Unbiased clustering was applied and correlation scores are depicted by heatmap. All MS samples were compared and clustered using standard artMS (https://github.com/biodavidjm/artMS) procedures on observed feature intensities computed by MaxQuant.

FIG. 28 shows representative data illustrating a correlation analysis of MERS-CoV proteomics samples. Pearson's pairwise correlations were calculated for all combinations of replicates of MERS-CoV affinity purification-mass spectrometry (AP-MS) samples. Unbiased clustering was applied and correlation scores are depicted by heatmap. All MS samples were compared and clustered using standard artMS (https://github.com/biodavidjm/artMS) procedures on observed feature intensities computed by MaxQuant.

FIG. 29 shows a representative illustration of the SARS-CoV-1 Virus-Human Protein Interaction Network. Virus-human protein-protein interaction map depicting high-confidence interactions (MiST≥0.7 & Saint BFDR≤0.05 & Average Spectral Counts≥2) for SARS-CoV-1 as derived from affinity purificationmass spectrometry (AP-MS) is shown. Viral bait proteins are depicted with orange diamonds and human proteins with dark grey circles. Human-human interactions are depicted in thin, dark grey lines. Proteins within the same protein complexes or biological process are indicated with light yellow or light blue highlighting, respectively, and annotated accordingly. Human-human physical interactions, protein complex definitions, and biological process groupings are derived from CORUM (M. Giurgiu, et al., CORUM: the comprehensive resource of mammalian protein complexes-2019. Nucleic Acids Res. 47, D559-D563 (2019)), Gene Ontology (biological process), and manually curated from literature sources.

FIG. 30 shows a representative illustration of the MERS-CoV Virus-Human Protein Interaction Network. Virus-human protein-protein interaction map depicting high-confidence interactions (MiST≥0.7 & Saint BFDR≤0.05 & Average Spectral Counts≥2) for MERS-CoV as derived from affinity purification-mass spectrometry (AP-MS) is shown. Viral bait proteins are depicted with yellow diamonds and human proteins with dark grey circles. Human-human interactions are depicted in thin, dark grey lines. Proteins within the same protein complexes or biological process are indicated with light yellow or light blue highlighting, respectively, and annotated accordingly. Human-human physical interactions, protein complex definitions, and biological process groupings are derived from CORUM (M. Giurgiu, et al., CORUM: the comprehensive resource of mammalian protein complexes-2019. Nucleic Acids Res. 47, D559-D563 (2019)), Gene Ontology (biological process), and manually curated from literature sources.

FIG. 31 shows a representative illustration of the SARS-CoV-2 Nsp16 Virus-Host Protein Interaction Network. Virus-human protein-protein interaction map depicting high-confidence interactions (MiST≥0.7 & Saint BFDR≤0.05 & Average Spectral Counts≥2) for SARS-CoV-2 Nsp16 protein is shown. This network is derived from affinity purification-mass spectrometry (AP-MS) data. Viral bait proteins are depicted with red diamonds and human proteins with dark grey circles. Human-human interactions are depicted in thin, dark grey lines. Proteins within the same protein complexes or biological process are indicated with light yellow or light blue highlighting, respectively, and annotated accordingly. Human-human physical interactions, protein complex definitions, and biological process groupings are derived from CORUM (M. Giurgiu, et al., CORUM: the comprehensive resource of mammalian protein complexes-2019. Nucleic Acids Res. 47, D559-D563 (2019)), Gene Ontology (biological process), and manually curated from literature sources.

FIG. 32 shows a representative flowchart illustrating the use of mass spectrometry to generate protein-protein interaction (PPI) maps, which can then be analyzed using differential interaction scoring (DIS) to identify novel drug targets and, thus, to develop novel drugs.

FIG. 33 shows a representative flowchart illustrating the use of mass spectrometry in combination with differential interaction scoring (DIS) to identify novel drug targets for viral diseases such as, for example, coronaviruses, which can then be used to develop novel therapeutics for treating these diseases.

FIG. 34 shows a representative flowchart illustrating the use of mass spectrometry in combination with differential interaction scoring (DIS) to identify novel drug targets for neurodegenerative diseases such as, for example, Amyotrophic Lateral Sclerosis (ALS), Parkinson's disease, and Alzheimer's disease (AD), which can then be used to develop novel therapeutics for treating these diseases.

FIG. 35 shows a representative flowchart illustrating the use of mass spectrometry in combination with differential interaction scoring (DIS) to identify novel drug targets for neuropsychiatric diseases such as, for example, autism, schizophrenia, obsessive compulsive disorder (OCD), anxiety, and depression, which can then be used to develop novel therapeutics for treating these diseases.

FIG. 36 shows a representative flowchart illustrating the use of mass spectrometry in combination with differential interaction scoring (DIS) to identify novel drug targets for cancers such as, for example, breast, head and neck, lung, pancreatic, and brain, which can then be used to develop novel chemotherapeutics.

FIG. 37 shows a representative flowchart illustrating the use of structural-biology techniques, such as cryoEM, in combination with artificial intelligence (AI) prediction based on deep neural networks to construct a 3-dimensional (3D) structure of a protein.

FIG. 38 shows a representative flowchart illustrating the architecture of the Alphafold system for predicting structure from protein sequence.

FIG. 39A shows that AI prediction by itself fails to recapitulate the correct global protein structure. Correct structure in black; top 6 scoring predictions based on the Alphafold system in grayscale; best RMSD 16 Å, average RMSD 34 Å. FIG. 39B shows that cryoEM by itself only yields low resolution density for full protein, preventing complete model from being constructed. Region which cannot be built solely based on cryoEM data is circled. FIG. 39C shows that the combination of the two methodologies (AI and cryoEM) yields high resolution structure for complete protein. The model obtained from cryoEM in black; the model obtained from AlphaFold prediction in grayscale.

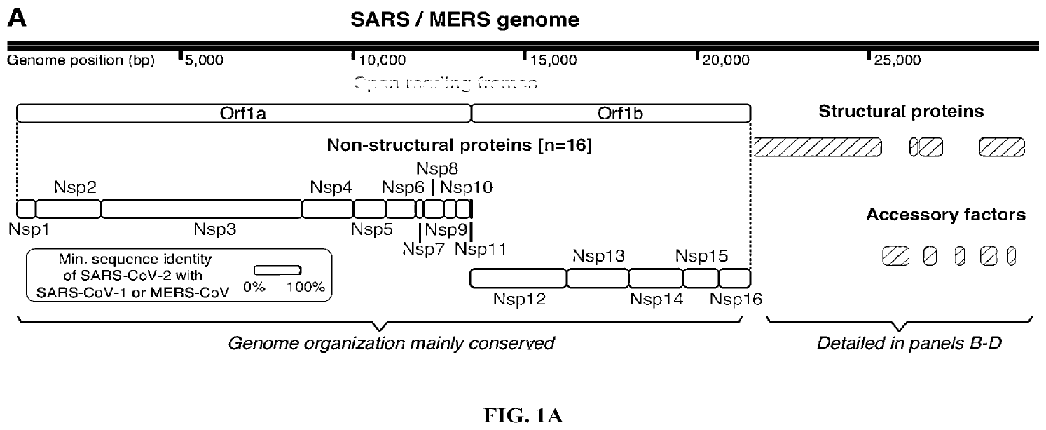

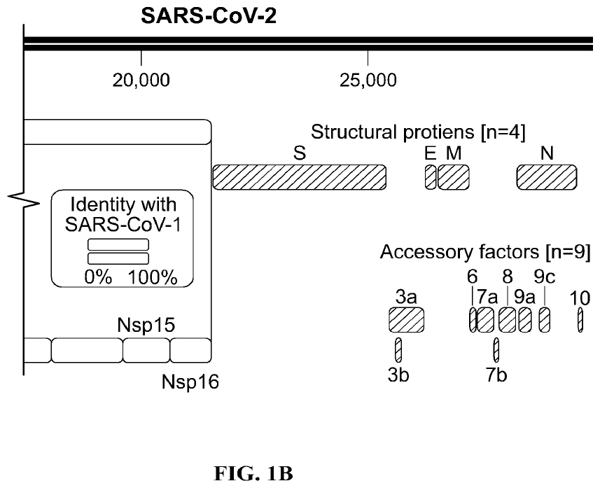

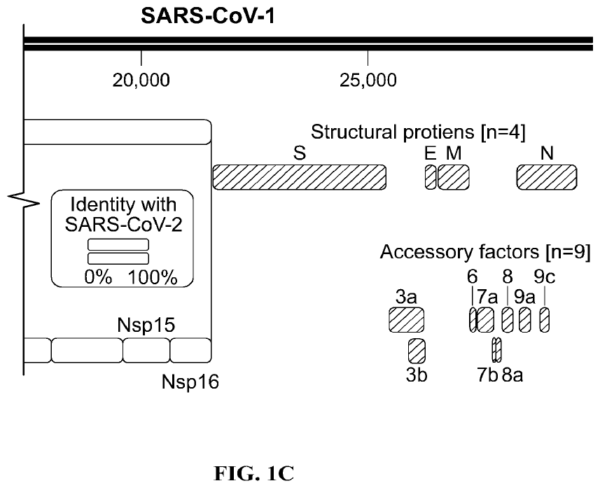

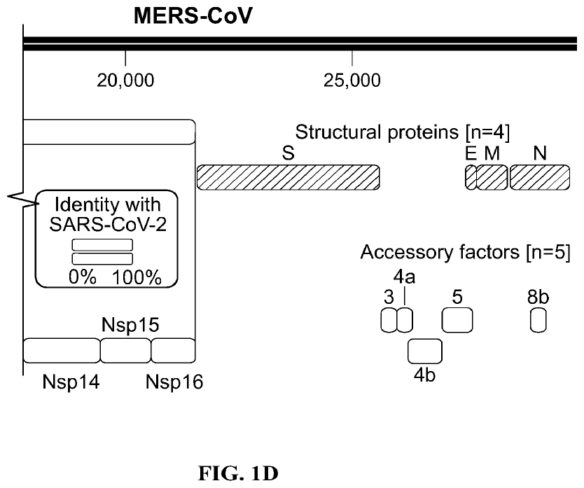

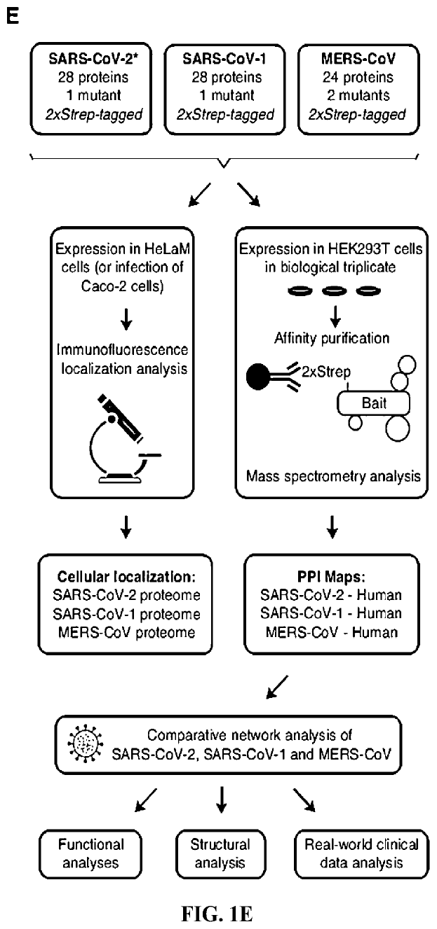

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF EMBODIMENTS

Before the present systems and methods are described, it is to be understood that the present disclosure is not limited to the particular processes, compositions, or methodologies described, as these may vary. It is also to be understood that the terminology used in the description is for the purposes of describing the particular versions or embodiments only, and is not intended to limit the scope of the present disclosure. Unless defined otherwise, all technical and scientific terms used herein have the same meanings as commonly understood by one of ordinary skill in the art. Although any methods and materials similar or equivalent to those described herein can be used in the practice or testing of embodiments of the present disclosure, the methods, devices, and materials in some embodiments are now described. All publications mentioned herein are incorporated by reference in their entirety. Nothing herein is to be construed as an admission that the present disclosure is not entitled to antedate such disclosure by virtue of prior invention.

### Definitions

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of.” “Consisting essentially of,” when used in the claims, shall have its ordinary meaning as used in the field of patent law.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value as such variations are appropriate to perform the disclosed methods. When “about” is present before a series of numbers or a range, it is understood that “about” can modify each of the numbers in the series or range.

The term “at least” prior to a number or series of numbers (e.g. “at least two”) is understood to include the number adjacent to the term “at least,” and all subsequent numbers or integers that could logically be included, as clear from context. When “at least” is present before a series of numbers or a range, it is understood that “at least” can modify each of the numbers in the series or range.

Ranges provided herein are understood to include all individual integer values and all subranges within the ranges.

As used herein, the terms “patient,” “individual diagnosed with . . . ,” and “individual suspected of having . . . ” all refer to an individual who has been diagnosed with a particular disease or a disorder (e.g., viral and bacterial diseases, cancers, neurodegenerative diseases, and neuropsychiatric disorders), has been given a probable diagnosis of a particular disease or disorder (e.g., viral and bacterial diseases, cancers, neurodegenerative diseases, and neuropsychiatric disorders), or an individual who has positive scans (e.g., PET scans) but otherwise lacks major symptoms of a particular disease or disorder and is without a clinical diagnosis of a disease disorder.

As used herein, the term “animal” includes, but is not limited to, humans and non-human vertebrates such as wild animals, rodents, such as rats, ferrets, and domesticated animals, and farm animals, such as dogs, cats, horses, pigs, cows, sheep, and goats. In some embodiments, the animal is a mammal. In some embodiments, the animal is a human. In some embodiments, the animal is a non-human mammal.

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

The term “diagnosis” or “prognosis” as used herein refers to the use of information (e.g., genetic information or data from other molecular tests on biological samples, signs and symptoms, physical exam findings, cognitive performance results, etc.) to anticipate the most likely outcomes, timeframes, and/or response to a particular treatment for a given disease, disorder, or condition, based on comparisons with a plurality of individuals sharing common nucleotide sequences, symptoms, signs, family histories, or other data relevant to consideration of a patient's health status.

As used herein, the phrase “in need thereof” means that the animal or mammal has been identified or suspected as having a need for the particular method or treatment. In some embodiments, the identification can be by any means of diagnosis or observation. In any of the methods and treatments described herein, the animal or mammal can be in need thereof. In some embodiments, the subject in need thereof is a human seeking prevention of a disease or disorder (e.g., viral and bacterial diseases, cancers, neurodegenerative diseases, and neuropsychiatric disorders). In some embodiments, the subject in need thereof is a human diagnosed with a disease or disorder (e.g., viral and bacterial diseases, cancers, neurodegenerative diseases, and neuropsychiatric disorders). In some embodiments, the subject in need thereof is a human seeking treatment for a disease or disorder (e.g., viral and bacterial diseases, cancers, neurodegenerative diseases, and neuropsychiatric disorders). In some embodiments, the subject in need thereof is a human undergoing treatment for a disease or disorder (e.g., viral and bacterial diseases, cancers, neurodegenerative diseases, and neuropsychiatric disorders).

As used herein, the term “mammal” means any animal in the class Mammalia such as rodent (i.e., mouse, rat, or guinea pig), monkey, cat, dog, cow, horse, pig, or human. In some embodiments, the mammal is a human. In some embodiments, the mammal refers to any non-human mammal. The present disclosure relates to any of the methods or compositions of matter wherein the sample is taken from a mammal or non-human mammal. The present disclosure relates to any of the methods or compositions of matter wherein the sample is taken from a human or non-human primate.

As used herein, the term “predicting” refers to making a finding that an individual has a significantly enhanced probability or likelihood of benefiting from and/or responding to a treatment for a disease or disorder (e.g., viral and bacterial diseases, cancers, neurodegenerative diseases, and neuropsychiatric disorders).

A “score” is a numerical value that may be assigned or generated after normalization of the value corresponding to protein-protein interactions associated with a particular disorder (e.g., viral and bacterial diseases, cancers, neurodegenerative diseases, and neuropsychiatric disorders). In some embodiments, the score is normalized in respect to a control data value, such as a value corresponding to a sample from a subject not exhibiting a mutation (e.g wildtype gene or protein from subject).

As used herein, the term “stratifying” refers to sorting individuals into different classes or strata based on the features of the particular disease or disorder (e.g., viral and bacterial diseases, cancers, neurodegenerative diseases, and neuropsychiatric disorders). For example, stratifying a population of individuals with a cancer involves assigning the individuals on the basis of the severity of the disease (e.g., stage 0, stage 1, stage, 2, stage 3, etc.).

As used herein, the term “subject,” “individual,” or “patient,” used interchangeably, means any animal, including mammals, such as mice, rats, other rodents, rabbits, dogs, cats, swine, cattle, sheep, horses, or primates, such as humans. In some embodiments, the subject is a human seeking treatment for a particular disease or disorder (e.g., viral and bacterial diseases, cancers, neurodegenerative diseases, and neuropsychiatric disorders). In some embodiments, the subject is a human diagnosed with a particular disease or disorder (e.g., viral and bacterial diseases, cancers, neurodegenerative diseases, and neuropsychiatric disorders). In some embodiments, the subject is a human suspected of having a particular disease or disorder (e.g., viral and bacterial diseases, cancers, neurodegenerative diseases, and neuropsychiatric disorders). In some embodiments, the subject is a healthy human being.

As used herein, the term “threshold” refers to a defined value by which a normalized score can be categorized. By comparing to a preset threshold, a normalized score can be classified based upon whether it is above or below the preset threshold.

As used herein, the terms “treat,” “treated,” or “treating” can refer to therapeutic treatment and/or prophylactic or preventative measures wherein the object is to prevent or slow down (lessen) an undesired physiological condition, disorder or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment can also include eliciting a clinically significant response without excessive levels of side effects. Treatment also includes prolonging survival as compared to expected survival if not receiving treatment.

As used herein, the term “therapeutic” means an agent utilized to treat, combat, ameliorate, prevent, or improve an unwanted condition or disease of a patient.

A “therapeutically effective amount” or “effective amount” of a composition is a predetermined amount calculated to achieve the desired effect, i.e., to treat, combat, ameliorate, prevent, or improve one or more symptoms of a disease or disorder (e.g., viral and bacterial diseases, cancers, neurodegenerative diseases, and neuropsychiatric disorders). The activity contemplated by the present methods includes both medical therapeutic and/or prophylactic treatment, as appropriate. The specific dose of a compound administered according to the present disclosure to obtain therapeutic and/or prophylactic effects will, of course, be determined by the particular circumstances surrounding the case, including, for example, the compound administered, the route of administration, and the condition being treated. It will be understood that the effective amount administered will be determined by the physician in the light of the relevant circumstances including the condition to be treated, the choice of compound to be administered, and the chosen route of administration, and therefore the above dosage ranges are not intended to limit the scope of the present disclosure in any way. A therapeutically effective amount of compounds of embodiments of the present disclosure is typically an amount such that when it is administered in a physiologically tolerable excipient composition, it is sufficient to achieve an effective systemic concentration or local concentration in the tissue.

### Methods of Developing Protein-Protein Interaction Maps and Identifying Protein-Protein Interactions

In some embodiments, the disclosure relates to methods of identifying an interaction between a pathogen protein and a host protein, the method comprising: (a) identifying a first pathogen protein that co-localizes with a first host protein in one or a plurality of bioassays; (b) calculating a differential interaction score (DIS) corresponding to a pathogen protein and a host protein in a sample; and (c) correlating the DIS with the likelihood that the dysfunctional protein-protein interaction is a causal agent of pathogenicity of the pathogen.

In some embodiments, the disclosure relates to methods of identifying an interaction between a first protein and a second protein, wherein the first protein is associated with a disorder of a subject, the method comprising: (a) identifying a first protein that co-localizes with the second protein in one or a plurality of bioassays; (b) calculating a differential interaction score (DIS) corresponding to the first protein and a second protein in a sample from the subject; and (c) correlating the DIS with the likelihood that the dysfunctional protein-protein interaction is a causal agent of pathogenicity of the pathogen. In some embodiments, the method further comprises the step of: compiling genetic data about a population of subjects that comprise a mutation in a nucleic acid sequence that encodes the first protein. In some embodiments, the one or plurality of bioassays comprises performing a mass spectrometry analysis on a sample associated with the disorder to identify dysfunctional protein-protein interactions associated with the disorder.

In some embodiments, the sample is a population of cells.

In some embodiments, the bioassay comprises one or a combination of: mass spectrometry analysis is performed on a plurality of samples from a population of subjects infected with the pathogen; siRNA knockdown analysis, CRISPR-mediated knockout analysis, infectivity analysis; and co-immunoprecipitation.

In some embodiments, the method further comprises the step of: compiling genetic data about a population of subjects that comprise a mutation in a nucleic acid sequence that encodes the first host protein.

In some embodiments, the one or plurality of bioassays comprises performing a mass spectrometry analysis on a sample associated with the disorder to identify dysfunctional protein-protein interactions associated with the disorder.

In some embodiments, each sample comprises a mixture of population of cells unaffected by the disorder and a population of cells expressing a mutation.

In some embodiments, the calculating comprises calculating one or more of a SAINTexpress algorithm score, a CompPASS algorithm score, and a MiST algorithm score. In some embodiments, the calculating comprises calculating a SAINTexpress algorithm score and a MiST algorithm score.

In some embodiments, the SAINTexpress algorithm score is calculated by a formula:

$$\text{?}$$

$$\text{?} \text{indicates text missing or illegible when filed}$$

wherein Xᵢⱼ is the spectral count for a prey protein i identified in a purification of bait j;
wherein λᵢⱼ is the mean count from a Poisson distribution representing true interaction;
wherein κᵢⱼ is the mean count from a Poisson distribution representing false interaction;
wherein π_{T} is the proportion of true interactions in the data; and wherein dot notation represents all relevant model parameters estimated from the data for the pair of prey i and bait j.

In some embodiments, the MiST algorithm score is calculated by a first formula:

$${A}_{b , i} = \frac{{\sum }_{r = 1}^{{N}_{B}} {Q}_{b , i , r}}{{N}_{R}}$$

wherein A_{b,i} is the abundance of a given bait-prey pair i,b; wherein Q_{b,i,r} is the quantity of bait-prey pair b,I in a replica r; and Nᵣ is the number of replicas; a second formula:

$${R}_{b , i} = \frac{{\sum }_{r = 1}^{{N}_{B}} {Q}_{b , i , r} \cdot log ( {Q}_{b , i , r} )}{{{log}_{2} ( {N}_{R} )}^{- 1}}$$

wherein R_{b,i} is the reproducibility of a given bait-prey pair b,I; and a third formula:

$${S}_{b , i} = \frac{{A}_{b , i}}{{\sum }_{b = 1}^{{N}_{B}} {A}_{b , i}}$$

wherein S_{b,i} is the specificity of a given bait-prey pair b, i; and wherein N_{B} is the number of baits.

In some embodiments, the CompPASS algorithm score is calculated by a Z-score formula pair:

$${\overline{X}}_{j} = \frac{\sum \text{?} {X}_{i , j}}{k} ; n = 1 , 2 , … m \qquad ( Eq . 1 )$$

$${Z}_{i , j} = \frac{{X}_{i , j} - {\overline{X}}_{j}}{{σ}_{i}} \qquad ( Eq . 2 )$$

$$\text{?} \text{indicates text missing or illegible when filed}$$

wherein X is the TSC; wherein i is the bait number; wherein j is the interactor; wherein n is which interactor is being considered; wherein k is the total number of baits; and wherein s is the standard deviation of the TSC mean; a S-score formula:

$${S}_{i , j} = \sqrt{( \frac{k}{\sum \text{?} {f}_{i , j}} ) {X}_{i , j}} ; {f}_{i , j} = \{ \begin{matrix}1 : {X}_{i , j} > 0 \\ {X}_{i , j}\end{matrix} \qquad ( Eq . 3 )$$

$$\text{?} \text{indicates text missing or illegible when filed}$$

wherein f is 0 or 1; a D-score formula:

$${D}_{i , j} = \sqrt{{( \frac{k}{\sum \text{?} {f}_{i , j}} )}^{p} {X}_{i , j}} ; \\ \begin{matrix}{f}_{i , j} = \{ \begin{matrix}1 : {X}_{i , j} > 0 \\ {X}_{i , j}\end{matrix} \\ p = \begin{matrix}number of replicates runs in \\ which the interactor is present\end{matrix}\end{matrix} \qquad ( Eq . 4 )$$

$$\text{?} \text{indicates text missing or illegible when filed}$$

wherein p is 1 or 2; and a WD-score formula:

$${WD}_{i , j} = \sqrt{{( \frac{k}{\sum \text{?} {f}_{i , j}} {ω}_{j} )}^{p} {X}_{i , j}} \\ {ω}_{i} = ( \frac{{σ}_{j}}{{\overline{X}}_{i}} ) , {\overline{X}}_{j} = \frac{\sum \text{?} {X}_{i , j}}{k} ; \\ n = 1 , 2 , … m , \begin{matrix}if {ω}_{j} \leq 1 \to {ω}_{j} = 1 \\ if {ω}_{j} > 1 \to {ω}_{j} = {ω}_{j}\end{matrix} \\ {f}_{i , j} = \{ \begin{matrix}1 : {X}_{i , j} > 0 \\ {X}_{i , j}\end{matrix} p = \begin{matrix}number of replicates runs in \\ which the interactor is present\end{matrix} \qquad ( Eq . 5 )$$

$$\text{?} \text{indicates text missing or illegible when filed}$$

wherein wⱼ is a weight factor; wherein σⱼ is a standard deviation.

In some embodiments, the DIS is calculated by a first formula:

DIS_{A}(b,p)=S_{C1}(b,p)×S_{C2}(b,p)×[1−S_{C3}(b,p)]

wherein DIS_{A}(b,p) is the DIS for each protein-protein interaction (PPI) (b, p) that is conserved in a first bioassay and a second bioassay, but not shared by a third bioassay; wherein S_{C1}(b,p) is the probability of a PPI being present in the first bioassay; wherein S_{C2}(b,p) is the probability of a PPI being present in the second bioassay; and wherein S_{c3}(b,p) is the probability of a PPI being present in the third bioassay; and a second formula:

DIS_{B}(b,p)=[1−S_{C1}(b,p)]×[1−S_{C2}(b,p)]×S_{C3}(b,p

wherein DIS_{B}(b,p) is the DIS score for each PPI (b, p) that is conserved in the third bioassay, but not shared by the first bioassay and the second bioassay; wherein a (+) sign is assigned if DIS_{A}(b,p)>DIS_{B}(b,p); and wherein a (−) sign is assigned if DIS_{A}(b,p)<DIS_{B}(b,p).

In some embodiments, the first, second and third bioassays are expression in a first cell line, expression in a second cell line and expression in a third cell line, respectively.

In some embodiments, the DIS is an average of a SAINTexpress algorithm score and a CompPASS algorithm score.

In some embodiments, the DIS comprises a SAINTexpress algorithm score.

In some embodiments, the DIS is from about 0.0 to about 1.0.

In some embodiments, a DIS of greater than about 0.5 indicates that the protein-protein interaction is likely a causal agent of the disorder.

In some embodiments, a DIS of less than about 0.5 indicates that the protein-protein interaction is not likely a causal agent of the disorder.

In some embodiments, the bioassay is a mass spectrometry analysis performed on a plurality of samples; and calculating comprises calculating a SAINTexpress algorithm score for each sample, and averaging the SAINTexpress algorithm scores.

In some embodiments, the pathogen is a virus. In some embodiments, the pathogen is selected from human immunodeficiency virus (HIV), human papillomavirus (HPV), chicken pox virus, infectious mononucleosis, mumps, measles, rubella, VSV, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, yellow fever virus, tick-borne encephalitis virus, Chikungunya virus (CHIKV), Venezuelan equine encephalitis (VEEV), Eastern equine encephalitis (EEEV), Western equine encephalitis (WEEV), dengue (DENY), influenza, West Nile virus (WNV), zika (ZIKV), Middle East Respiratory Syndromes (MERS), Severe Acute Respiratory Syndrome (SARS), and coronavirus disease 2019 (COVID-19).

In some embodiments, the pathogen protein is from Coronaviridae. In some embodiments, the pathogen protein is expressed by one of: Middle East Respiratory Syndromes coronavirus (MERS-CoV), Severe Acute Respiratory Syndrome coronavirus (SARS-CoV), and SARS-CoV-2.

In some embodiments, the protein-protein interaction is an Orf9b: Tom70 interaction or an Orf8: IL17RA interaction.

In some embodiments, the host protein is human prostaglandin E synthase type 2 (PGES-2) or a human sigma receptor.

In some embodiments, the bioassay comprises one or a combination of: mass spectrometry analysis is performed on a plurality of samples from a population of subjects infected with the pathogen; siRNA knockdown analysis, CRISPR-mediated knockout analysis, infectivity analysis; and co-immunoprecipitation.

In some embodiments, the method further comprises the step of: compiling genetic data about a population of subjects that comprise a mutation in a nucleic acid sequence that encodes the first protein.

In some embodiments, the one or plurality of bioassays comprises performing a mass spectrometry analysis on a sample associated with the disorder to identify dysfunctional protein-protein interactions associated with the disorder.

In some embodiments, a nucleic acid that encodes the first protein comprises at least about 70% sequence identity to any one of the nucleic acids identified in Table X.

In some embodiments, the disorder is a cancer. In some embodiments, the cancer is a sarcoma, a carcinoma, a hematological cancer, a solid tumor, breast cancer, cervical cancer, gastrointestinal cancer, colorectal cancer, brain cancer, skin cancer, head and neck cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, or plasma cell neoplasm (myeloma). In some embodiments, the cancer is breast cancer, head and neck cancer, lung cancer, pancreatic cancer, or brain cancer.

In some embodiments, the disorder is a neuropsychiatric disease. In some embodiments, the neuropsychiatric disorder is autism, schizophrenia, obsessive-compulsive disorder (OCD), anxiety, depression, migraine headaches, palsies, seizures, addiction, uncontrolled anger, anorexia nervosa, bulimia nervosa, binge-eating disorder, attention deficit disorder (ADD), or attention-deficit/hyperactivity disorder (ADHD).

In some embodiments, the neuropsychiatric disorder is autism, schizophrenia, obsessive-compulsive disorder (OCD), anxiety, or depression. In some embodiments, the disorder is a neurodegenerative disease.

In some embodiments, the neurodegenerative disease is amytrophic lateral sclerosis (ALS), Parkinson's disease, Alzheimer's disease, Prion disease, motor neurone diseases (MND), Huntington's disease, spinocerebellar ataxia (SCA), or spinal muscular atrophy (SMA).

In some embodiments, the neurodegenerative disease is amytrophic lateral sclerosis (ALS), Parkinson's disease, or Alzheimer's disease.

In some embodiments, the method further comprises harvesting samples with a functional bioassay. In some embodiments, the functional bioassay is an animal model comprising growth of transformed cell lines.

In some embodiments, the disorder is a viral disease that is due to a Coronavirus, and wherein the disorder treatment comprises administration of a prostaglandin E synthase type 2 (PGES-2) inhibitor or a sigma receptor inhibitor.

In some embodiments, the sigma receptor inhibitor is an antipsychotic (e.g., fluphenazine, chlorpromazine, haloperidol), an antihistamine (e.g., clemastine, meclizine), an antimalarial (e.g., hydroxychloroquine, chloroquine), amiodarone, tamoxifen, triparanol, clomiphene, or propranalol.

In some embodiments, the method further comprises a step of mapping the spatial organization of the protein-protein interaction.

In some embodiments, the method further comprises a step of validating the protein-protein interaction by performing one or combination of: X-ray crystallography, mass spectrometry, and electron microscopy.

In some embodiments, the electron microscopy is cryogenic electron microscopy.

In some embodiments, the disclosure relates to methods of imaging a protein, the method comprising: (a) identifying a first protein that co-localizes with a first host protein in one or a plurality of bioassays; (b) calculating a differential interaction score (DIS) corresponding to the first protein in a sample; and (c) predicting the three-dimensional structure of the first protein by integrating the DIS score into a fit of cryo-EM structure image. In some embodiments, the first protein is isolated in vitro from a sample. In some embodiments, the sample is from a cell extract or subject. In some embodiments, the first protein is mutated as compared to a wild-type or endogenous, unmutated sequence. In some embodiments, the method is a computer-implemented method performed on a system disclosed herein, comprising instructions for execution of the DIS calculation.

In some embodiments, the disclosure relates to methods of imaging an interaction between a pathogen protein and a host protein, the method comprising: (a) identifying a first pathogen protein that co-localizes with a first host protein in one or a plurality of bioassays; (b) calculating a differential interaction score (DIS) corresponding to a pathogen protein and a host protein in a sample; and (c) correlating the DIS with the likelihood that the dysfunctional protein-protein interaction is a causal agent of pathogenicity of the pathogen.

In some embodiments, the disclosure relates to methods of imaging an interaction between a first protein and a second protein, wherein the first protein is associated with a disorder of a subject, the method comprising: (a) identifying a first protein that co-localizes with the second protein in one or a plurality of bioassays; (b) calculating a differential interaction score (DIS) corresponding to the first protein and a second protein in a sample from the subject; and (c) correlating the DIS with the likelihood that the dysfunctional protein-protein interaction is a causal agent of pathogenicity of the pathogen. In some embodiments, the method further comprises the step of: compiling genetic data about a population of subjects that comprise a mutation in a nucleic acid sequence that encodes the first protein. In some embodiments, the one or plurality of bioassays comprises performing a mass spectrometry analysis on a sample associated with the disorder to identify dysfunctional protein-protein interactions associated with the disorder.

In some embodiments, the method further comprises applying Cryo-EM as described elsewhere herein, thereby providing a 3-dimensional structure of the interaction. For example, in some embodiments, the method further comprises: (a) obtaining a molecular volume for the first protein while co-localized with the second protein using a structural-biology technique at a resolution of about 20 Å or better (less); (b) predicting a 3D structure of the first protein co-localized with the second protein based on artificial intelligence (AI) prediction using one or a plurality of deep neural networks to predict the 3D structure based on sequence; (c) breaking the 3D structure predicted in step (b) into overlapping regions; (d) global rigid-body fitting the overlapping regions against the molecular volume obtained in step (a); (e) examining top scoring fits and generating new region boundaries; (f) optionally repeating steps (d) and (e) for one or a plurality of times; (g) combining the regions into a complete protein-protein structure; and (h) refining the complete protein-protein structure obtained in step (g) into the molecular volume of (a). In some embodiments, the method further comprises applying Cryo-EM as described elsewhere herein, thereby providing a 3-dimensional structure of the interaction. For example, in some embodiments, the method further comprises: (a) obtaining a molecular volume for the first protein while co-localized with the second protein using a structural-biology technique; (b) predicting a 3D structure of the first protein co-localized with the second protein based on artificial intelligence (AI) prediction; (c) breaking the 3D structure predicted in step (b) into overlapping regions; (d) global rigid-body fitting the overlapping regions against the molecular volume obtained in step (a); and (e) examining top scoring fits and generating new region boundaries. In some embodiments, the method further comprises generating a structural image of the first protein and/or second protein based upon any one or more of steps (a), (b), (c), (d) and (e). In some embodiments, the AI prediction is performed by applying one or a plurality of deep neural networks to predict the 3D structure based on amino acid sequence. In some embodiments, the AI prediction is performed by using AlphaFold (available at https://alphafold.ebi.ac.uk, which is incorporated by reference in its entirety). In some embodiments, the methods further comprise optionally repeating steps (d) and (e) for one or a plurality of times. In some embodiments, the methods further comprise (g) combining the regions into a complete protein-protein structure. In some embodiments the methods further comprise (h) refining the complete protein-protein structure obtained in step (g) into the molecular volume of (a). In some embodiments, the methods further comprise imaging the complete protein-protein structure by using a computer program product in a system operably connected to or part of a controller in a system disclosed herein, such system comprising a display operably connected to the controller and capable of displaying the complete protein-protein structure to an operator of the system. In some embodiments, the methods are computer-implemented methods comprising a step of calculating a DIS.

In some embodiments, the disclosed methods further comprise creating a genetic interaction phenotypic profile. Genetic interaction phenotypic profiles are disclosed in PCT/US21/55059, the contents of which are hereby incorporated by reference.

### Methods of Identifying Therapeutic Targets and of Screening for and Evaluating Therapeutics

In some embodiments, the disclosure relates to methods of identifying a therapeutic target for a disorder treatment, the method comprising: (a) compiling genetic data about a population of subjects that has a mutation candidate that causes a disorder; (b) performing a mass spectrometry analysis on a sample associated with the disorder to identify dysfunctional protein-protein interactions associated with the disorder; (c) calculating a differential interaction score (DIS); (d) correlating the DIS with the likelihood that the dysfunctional protein-protein interaction is a causal agent of the disorder, wherein if the DIS score is above a first threshold, then the causal agent is selected as a therapeutic target for the disorder treatment, and wherein if the DIS score is below the first threshold, then the causal agent is not selected as a therapeutic target for the disorder treatment.

In some embodiments, the disclosure relates to methods of identifying a therapeutic target for a disorder treatment, the method comprising: (a) calculating a differential interaction score (DIS); and (b) correlating the DIS with a likelihood that a dysfunctional protein-protein interaction is a causal agent of the disorder, wherein if the DIS score is above a first threshold, then the causal agent is selected as a therapeutic target for the disorder treatment, and wherein if the DIS score is below the first threshold, then the causal agent is not selected as a therapeutic target for the disorder treatment.

In some embodiments, the disclosure relates to methods of identifying a therapeutic for treating a disorder, the method comprising screening a candidate compound for binding with, or activity against a therapeutic target, wherein the therapeutic target was identified via a disclosed method.

In some embodiments, the disclosure relates to methods of predicting a likelihood that a disorder is responsive to a therapeutic, the method comprising: (a) compiling genetic data about a population of subjects that has a mutation candidate that causes a disorder; (b) performing a mass spectrometry analysis on a sample associated with the disorder to identify dysfunctional protein-protein interactions associated with the disorder; (c) calculating a differential interaction score (DIS); (d) correlating the DIS with the likelihood that the dysfunctional protein-protein interaction is the causal agent of the disorder; and (e) selecting a therapeutic for treating the disorder based upon the causal agent.

In some embodiments, the sample is a population of cells.

In some embodiments, the bioassay comprises one or a combination of: mass spectrometry analysis is performed on a plurality of samples from a population of subjects infected with the pathogen; siRNA knockdown analysis, CRISPR-mediated knockout analysis, infectivity analysis; and co-immunoprecipitation.

In some embodiments, the method further comprises the step of: compiling genetic data about a population of subjects that comprise a mutation in a nucleic acid sequence that encodes the first host protein.

In some embodiments, the one or plurality of bioassays comprises performing a mass spectrometry analysis on a sample associated with the disorder to identify dysfunctional protein-protein interactions associated with the disorder.

In some embodiments, each sample comprises a mixture of population of cells unaffected by the disorder and a population of cells expressing a mutation.

In some embodiments, the calculating comprises calculating one or more of a SAINTexpress algorithm score, a CompPASS algorithm score, and a MiST algorithm score as further described elsewhere herein. In some embodiments, the calculating comprises calculating a SAINTexpress algorithm score and a MiST algorithm score.

In some embodiments, the DIS is calculated by a first formula:

DIS_{A}(b,p)=S_{C1}(b,p)×S_{C2}(b,p)×[1−S_{C3}(b,p)]

wherein DIS_{A}(b,p) is the DIS for each protein-protein interaction (PPI) (b, p) that is conserved in a first bioassay and a second bioassay, but not shared by a third bioassay; wherein S_{C1}(b,p) is the probability of a PPI being present in the first bioassay; wherein S_{C2}(b,p) is the probability of a PPI being present in the second bioassay; and wherein S_{c3}(b,p) is the probability of a PPI being present in the third bioassay; and a second formula:

DIS_{B}(b,p)=[1−S_{C1}(b,p)]×[1−S_{C2}(b,p)]×S_{C3}(b,p)

wherein DIS_{B}(b,p) is the DIS score for each PPI (b, p) that is conserved in the third bioassay, but not shared by the first bioassay and the second bioassay; wherein a (+) sign is assigned if DIS_{A}(b,p)>DIS_{B}(b,p); and wherein a (−) sign is assigned if DIS_{A}(b,p)<DIS_{B}(b,p).

In some embodiments, the first, second and third bioassays are expression in a first cell line, expression in a second cell line and expression in a third cell line, respectively.

In some embodiments, the DIS is an average of a SAINTexpress algorithm score and a CompPASS algorithm score.

In some embodiments, the DIS comprises a SAINTexpress algorithm score.

In some embodiments, the DIS is from about 0.0 to about 1.0.

In some embodiments, a DIS of greater than about 0.5 indicates that the protein-protein interaction is likely a causal agent of the disorder.

In some embodiments, a DIS of less than about 0.5 indicates that the protein-protein interaction is not likely a causal agent of the disorder.

In some embodiments, the bioassay is a mass spectrometry analysis performed on a plurality of samples; and calculating comprises calculating a SAINTexpress algorithm score for each sample, and averaging the SAINTexpress algorithm scores.

In some embodiments, the pathogen is a virus. In some embodiments, the pathogen is selected from human immunodeficiency virus (HIV), human papillomavirus (HPV), chicken pox virus, infectious mononucleosis, mumps, measles, rubella, VSV, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, yellow fever virus, tick-borne encephalitis virus, Chikungunya virus (CHIKV), Venezuelan equine encephalitis (VEEV), Eastern equine encephalitis (EEEV), Western equine encephalitis (WEEV), dengue (DENY), influenza, West Nile virus (WNV), zika (ZIKV), Middle East Respiratory Syndromes (MERS), Severe Acute Respiratory Syndrome (SARS), and coronavirus disease 2019 (COVID-19).

In some embodiments, the pathogen protein is from Coronaviridae. In some embodiments, the pathogen protein is expressed by one of: Middle East Respiratory Syndromes coronavirus (MERS-CoV), Severe Acute Respiratory Syndrome coronavirus (SARS-CoV), and SARS-CoV-2.

In some embodiments, the protein-protein interaction is an Orf9b: Tom70 interaction or an Orf8: IL17RA interaction.

In some embodiments, the host protein is human prostaglandin E synthase type 2 (PGES-2) or a human sigma receptor.

In some embodiments, the bioassay comprises one or a combination of: mass spectrometry analysis is performed on a plurality of samples from a population of subjects infected with the pathogen; siRNA knockdown analysis, CRISPR-mediated knockout analysis, infectivity analysis; and co-immunoprecipitation.

In some embodiments, the method further comprises the step of: compiling genetic data about a population of subjects that comprise a mutation in a nucleic acid sequence that encodes the first protein.

In some embodiments, the one or plurality of bioassays comprises performing a mass spectrometry analysis on a sample associated with the disorder to identify dysfunctional protein-protein interactions associated with the disorder.

In some embodiments, a nucleic acid that encodes the first protein comprises at least about 70% sequence identity to any one of the nucleic acids identified in Table X.

In some embodiments, the disorder is a cancer. In some embodiments, the cancer is a sarcoma, a carcinoma, a hematological cancer, a solid tumor, breast cancer, cervical cancer, gastrointestinal cancer, colorectal cancer, brain cancer, skin cancer, head and neck cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, or plasma cell neoplasm (myeloma). In some embodiments, the cancer is breast cancer, head and neck cancer, lung cancer, pancreatic cancer, or brain cancer.

In some embodiments, the disorder is a neuropsychiatric disease. In some embodiments, the neuropsychiatric disorder is autism, schizophrenia, obsessive-compulsive disorder (OCD), anxiety, depression, migraine headaches, palsies, seizures, addiction, uncontrolled anger, anorexia nervosa, bulimia nervosa, binge-eating disorder, attention deficit disorder (ADD), or attention-deficit/hyperactivity disorder (ADHD).

In some embodiments, the neuropsychiatric disorder is autism, schizophrenia, obsessive-compulsive disorder (OCD), anxiety, or depression. In some embodiments, the disorder is a neurodegenerative disease.

In some embodiments, the neurodegenerative disease is amytrophic lateral sclerosis (ALS), Parkinson's disease, Alzheimer's disease, Prion disease, motor neurone diseases (MND), Huntington's disease, spinocerebellar ataxia (SCA), or spinal muscular atrophy (SMA).

In some embodiments, the neurodegenerative disease is amytrophic lateral sclerosis (ALS), Parkinson's disease, or Alzheimer's disease.

In some embodiments, the method further comprises harvesting samples with a functional bioassay. In some embodiments, the functional bioassay is an animal model comprising growth of transformed cell lines.

In some embodiments, the disorder is a viral disease that is due to a Coronavirus, and wherein the disorder treatment comprises administration of a prostaglandin E synthase type 2 (PGES-2) inhibitor or a sigma receptor inhibitor.

In some embodiments, the sigma receptor inhibitor is an antipsychotic (e.g., fluphenazine, chlorpromazine, haloperidol), an antihistamine (e.g., clemastine, meclizine), an antimalarial (e.g., hydroxychloroquine, chloroquine), amiodarone, tamoxifen, triparanol, clomiphene, or propranalol.

In some embodiments, the step of identifying the genetic information from a subject comprises sequencing the genetic information from a biopsy or sample obtained from the subject.

In some embodiments, the first, second and third cell lines are cell lines used in performance of a functional bioassay.

In some embodiments, the step of selecting a disorder treatment comprises selecting a treatment from a database of known treatments for the dysfunctional protein-protein interaction.

In some embodiments, the method further comprises a step of mapping the spatial organization of the protein-protein interaction.

In some embodiments, the method further comprises a step of validating the protein-protein interaction by performing one or combination of: X-ray crystallography, mass spectrometry, and electron microscopy.

In some embodiments, the electron microscopy is cryogenic electron microscopy.

### Methods of Identifying and Monitoring a Subject's Responsiveness to a Hyperproliferative Disorder Treatment

In some embodiments, the disclosure relates to methods of identifying a subject likely to respond to a disorder treatment, the method comprising: (a) calculating a differential interaction score (DIS); and (b) correlating the DIS with a likelihood that a dysfunctional protein-protein interaction is a causal agent of the disorder, wherein if the DIS score is above a first threshold, then the subject is likely to respond to a disorder treatment based upon the causal agent, and wherein if the DIS score is below the first threshold, then the subject is not likely to respond to the disorder treatment based upon the causal agent. In some embodiments, the method further comprises (a) compiling genetic data about a population of subjects comprising the subject, wherein the population of subjects has a mutation candidate that causes the disorder; and (b) performing a mass spectrometry analysis on a sample associated with the disorder to identify dysfunctional protein-protein interactions associated with the disorder.

In some embodiments, the disclosure relates to methods of predicting a likelihood that a subject does or does not respond to a disorder treatment, the method comprising: (a) compiling genetic data about a population of subjects that has a mutation candidate that causes a disorder, wherein the population of subjects includes the subject; (b) performing a mass spectrometry analysis on a sample associated with the disorder to identify dysfunctional protein-protein interactions associated with the disorder; (c) calculating a differential interaction score (DIS); (d) correlating the DIS with the likelihood that the dysfunctional protein-protein interaction is the causal agent of the disorder; and (e) selecting a treatment for the subject based upon the causal agent. In some embodiments, the method further comprises: (f) comparing the DIS score to a first threshold; and (g) classifying the subject as being likely to respond to a disorder treatment, wherein each of steps (f) and (g) are performed after step (c), and wherein the first threshold is calculated relative to a first control dataset.

In some embodiments, the disclosure relates to methods of treating a viral infection due to a Coronavirus in a subject having a genetic alteration in PGES-2 signaling, the method comprising administering to the subject a pharmaceutically effective amount of a PGES-2 inhibitor, wherein the subject was previously identified as being in need of treatment by: (a) performing a mass spectrometry analysis on a sample from the subject; (b) identifying dysfunctional protein-protein interactions associated with the viral infection; and (c) calculating a differential interaction score (DIS).

In some embodiments, the disclosure relates to methods of treating a Coronaviridae viral infection in a subject in need thereof, the method comprising administering to the subject a pharmaceutically effective amount of a sigma receptor inhibitor, wherein the subject was previously identified as being in need of treatment by: (a) performing a mass spectrometry analysis on a sample from the subject; (b) identifying dysfunctional protein-protein interactions associated with the viral infection; and (c) calculating a differential interaction score (DIS).

In some embodiments, the disclosure relates to methods of selecting a disorder treatment for a subject in need thereof, the method comprising: (a) identifying genetic data from the subject in need of treatment; (b) comparing the genetic data from the subject to a compilation of genetic data from population of subjects that has a mutation candidate that causes a disorder, wherein the population of subjects includes the subject in need thereof; (c) performing a mass spectrometry analysis on a sample from the subject associated with the disorder to identify dysfunctional protein-protein interactions associated with the disorder; (d) calculating a differential interaction score (DIS); (e) correlating the DIS with the likelihood that the dysfunctional protein-protein interaction is a causal agent of the disorder; and (f) selecting a disorder treatment for the subject based upon the causal agent.

In some embodiments, the sample is a population of cells.

In some embodiments, the bioassay comprises one or a combination of: mass spectrometry analysis is performed on a plurality of samples from a population of subjects infected with the pathogen; siRNA knockdown analysis, CRISPR-mediated knockout analysis, infectivity analysis; and co-immunoprecipitation.

In some embodiments, the method further comprises the step of: compiling genetic data about a population of subjects that comprise a mutation in a nucleic acid sequence that encodes the first host protein.

In some embodiments, the one or plurality of bioassays comprises performing a mass spectrometry analysis on a sample associated with the disorder to identify dysfunctional protein-protein interactions associated with the disorder.

In some embodiments, each sample comprises a mixture of population of cells unaffected by the disorder and a population of cells expressing a mutation.

In some embodiments, the calculating comprises calculating one or more of a SAINTexpress algorithm score, a CompPASS algorithm score, and a MiST algorithm score as further described elsewhere herein. In some embodiments, the calculating comprises calculating a SAINTexpress algorithm score and a MiST algorithm score.

In some embodiments, the DIS is calculated by a first formula:

DIS_{A}(b,p)=S_{C1}(b,p)×S_{C2}(b,p)×[1−S_{C3}(b,p)]

wherein DIS_{A}(b,p) is the DIS for each protein-protein interaction (PPI) (b, p) that is conserved in a first bioassay and a second bioassay, but not shared by a third bioassay; wherein S_{C1}(b,p) is the probability of a PPI being present in the first bioassay; wherein S_{C2}(b,p) is the probability of a PPI being present in the second bioassay; and wherein S_{c3}(b,p) is the probability of a PPI being present in the third bioassay; and a second formula:

DIS_{B}(b,p)=[1−S_{C1}(b,p)]×[1−S_{C2}(b,p)]×S_{C3}(b,p)

wherein DIS_{B}(b,p) is the DIS score for each PPI (b, p) that is conserved in the third bioassay, but not shared by the first bioassay and the second bioassay; wherein a (+) sign is assigned if DIS_{A}(b,p)>DIS_{B}(b,p); and wherein a (−) sign is assigned if DIS_{A}(b,p)<DIS_{B}(b,p).

In some embodiments, the first, second and third bioassays are expression in a first cell line, expression in a second cell line and expression in a third cell line, respectively.

In some embodiments, the DIS is an average of a SAINTexpress algorithm score and a CompPASS algorithm score.

In some embodiments, the DIS comprises a SAINTexpress algorithm score.

In some embodiments, the DIS is from about 0.0 to about 1.0.

In some embodiments, a DIS of greater than about 0.5 indicates that the protein-protein interaction is likely a causal agent of the disorder.

In some embodiments, a DIS of less than about 0.5 indicates that the protein-protein interaction is not likely a causal agent of the disorder.

In some embodiments, the bioassay is a mass spectrometry analysis performed on a plurality of samples; and calculating comprises calculating a SAINTexpress algorithm score for each sample, and averaging the SAINTexpress algorithm scores.

In some embodiments, the pathogen is a virus. In some embodiments, the pathogen is selected from human immunodeficiency virus (HIV), human papillomavirus (HPV), chicken pox virus, infectious mononucleosis, mumps, measles, rubella, VSV, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, yellow fever virus, tick-borne encephalitis virus, Chikungunya virus (CHIKV), Venezuelan equine encephalitis (VEEV), Eastern equine encephalitis (EEEV), Western equine encephalitis (WEEV), dengue (DENY), influenza, West Nile virus (WNV), zika (ZIKV), Middle East Respiratory Syndromes (MERS), Severe Acute Respiratory Syndrome (SARS), and coronavirus disease 2019 (COVID-19).

In some embodiments, the pathogen protein is from Coronaviridae. In some embodiments, the pathogen protein is expressed by one of: Middle East Respiratory Syndromes coronavirus (MERS-CoV), Severe Acute Respiratory Syndrome coronavirus (SARS-CoV), and SARS-CoV-2.

In some embodiments, the protein-protein interaction is an Orf9b: Tom70 interaction or an Orf8: IL17RA interaction.

In some embodiments, the host protein is human prostaglandin E synthase type 2 (PGES-2) or a human sigma receptor.

In some embodiments, the bioassay comprises one or a combination of: mass spectrometry analysis is performed on a plurality of samples from a population of subjects infected with the pathogen; siRNA knockdown analysis, CRISPR-mediated knockout analysis, infectivity analysis; and co-immunoprecipitation.

In some embodiments, the method further comprises the step of: compiling genetic data about a population of subjects that comprise a mutation in a nucleic acid sequence that encodes the first protein.

In some embodiments, the one or plurality of bioassays comprises performing a mass spectrometry analysis on a sample associated with the disorder to identify dysfunctional protein-protein interactions associated with the disorder.

In some embodiments, a nucleic acid that encodes the first protein comprises at least about 70% sequence identity to any one of the nucleic acids identified in Table X.

In some embodiments, the disorder is a cancer. In some embodiments, the cancer is a sarcoma, a carcinoma, a hematological cancer, a solid tumor, breast cancer, cervical cancer, gastrointestinal cancer, colorectal cancer, brain cancer, skin cancer, head and neck cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, or plasma cell neoplasm (myeloma). In some embodiments, the cancer is breast cancer, head and neck cancer, lung cancer, pancreatic cancer, or brain cancer.

In some embodiments, the disorder is a neuropsychiatric disease. In some embodiments, the neuropsychiatric disorder is autism, schizophrenia, obsessive-compulsive disorder (OCD), anxiety, depression, migraine headaches, palsies, seizures, addiction, uncontrolled anger, anorexia nervosa, bulimia nervosa, binge-eating disorder, attention deficit disorder (ADD), or attention-deficit/hyperactivity disorder (ADHD).

In some embodiments, the neuropsychiatric disorder is autism, schizophrenia, obsessive-compulsive disorder (OCD), anxiety, or depression. In some embodiments, the disorder is a neurodegenerative disease.

In some embodiments, the neurodegenerative disease is amytrophic lateral sclerosis (ALS), Parkinson's disease, Alzheimer's disease, Prion disease, motor neurone diseases (MND), Huntington's disease, spinocerebellar ataxia (SCA), or spinal muscular atrophy (SMA).

In some embodiments, the neurodegenerative disease is amytrophic lateral sclerosis (ALS), Parkinson's disease, or Alzheimer's disease.

In some embodiments, the method further comprises harvesting samples with a functional bioassay. In some embodiments, the functional bioassay is an animal model comprising growth of transformed cell lines.

In some embodiments, the subject is a mammal. In some embodiments, the mammal is a human.

In some embodiments, the subject has been diagnosed with a need for treatment of the disorder prior to the administering step.

In some embodiments, the method further comprises identifying a subject in need of treatment of the disorder.

In some embodiments, the subject is identified as being likely to respond to a treatment if the DIS score is greater than 0.5.

In some embodiments, the subject is identified as being unlikely to respond to a treatment if the DIS score is 0.5 or less.

In some embodiments, the method further comprises selecting a disorder treatment for the subject based upon the interaction between the first and second protein.

In some embodiments, the disorder is a viral disease that is due to a Coronavirus, and wherein the disorder treatment comprises administration of a prostaglandin E synthase type 2 (PGES-2) inhibitor or a sigma receptor inhibitor.

In some embodiments, the sigma receptor inhibitor is an antipsychotic (e.g., fluphenazine, chlorpromazine, haloperidol), an antihistamine (e.g., clemastine, meclizine), an antimalarial (e.g., hydroxychloroquine, chloroquine), amiodarone, tamoxifen, triparanol, clomiphene, or propranalol.

In some embodiments, the subject comprises a genetic alteration in sigma receptor signaling.

In some embodiments, the step of identifying the genetic information from a subject comprises sequencing the genetic information from a biopsy or sample obtained from the subject.

In some embodiments, the first, second and third cell lines are cell lines used in performance of a functional bioassay.

In some embodiments, the step of selecting a disorder treatment comprises selecting a treatment from a database of known treatments for the dysfunctional protein-protein interaction.

In some embodiments, the method further comprises a step of mapping the spatial organization of the protein-protein interaction.

In some embodiments, the method further comprises a step of validating the protein-protein interaction by performing one or combination of: X-ray crystallography, mass spectrometry, and electron microscopy.

In some embodiments, the electron microscopy is cryogenic electron microscopy.

### Systems

The above-described methods can be implemented in any of numerous ways. For example, the embodiments may be implemented using a computer program product (i.e., software), hardware, software, or a combination thereof. When implemented in software, the software code can be executed on any suitable processor or collection of processors, whether provided in a single computer or distributed among multiple computers.

In some embodiments, the disclosure relates to computer program products encoded on a computer-readable storage medium, wherein the computer program product comprises instructions for: (a) identifying protein-protein interactions associated with the disorder; and (b) calculating a differential interaction score (DIS).

In some embodiments, the disclosure relates to systems for identifying a protein interaction network in a subject, the system comprising: (a) a processor operable to execute programs; (b) a memory associated with the processor; (c) a database associated with said processor and said memory; and (d) a program stored in the memory and executable by the processor, the program being operable for: (i) performing a mass spectrometry analysis on a sample from a subject that has a mutation candidate that causes a disorder; (ii) identifying dysfunctional protein-protein interactions associated with the disorder; and (iii) calculating a differential interaction score (DIS).

In some embodiments, the instructions further comprise a step of correlating the DIS with the likelihood that the dysfunctional protein-protein interaction is a causal agent of the disorder.

In some embodiments, the computer program product further comprise instructions for selecting a treatment for the subject based upon the causal agent.

In some embodiment, the computer program product further comprises instructions for: (d) comparing the DIS score to a first threshold; and (e) classifying the subject as being likely to respond to a disorder treatment, wherein each of steps (d) and (e) are performed after step (c), and wherein the first threshold is calculated relative to a first control dataset.

In some embodiments, disclosed is a system comprising a disclosed computer program product, and one or more of: (a) a processor operable to execute programs; and (b) a memory associated with the processor.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone, or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks, or fiber optic networks.

A computer employed to implement at least a portion of the functionality described herein may include a memory, coupled to one or more processing units (also referred to herein simply as “processors”), one or more communication interfaces, one or more display units, and one or more user input devices. The memory may include any computer-readable media, and may store computer instructions (also referred to herein as “processor-executable instructions”) for implementing the various functionalities described herein. The processing unit(s) may be used to execute the instructions. The communication interface(s) may be coupled to a wired or wireless network, bus, or other communication means and may therefore allow the computer to transmit communications to and/or receive communications from other devices. The display unit(s) may be provided, for example, to allow a user to view various information in connection with execution of the instructions. The user input device(s) may be provided, for example, to allow the user to make manual adjustments, make selections, enter data or various other information, and/or interact in any of a variety of manners with the processor during execution of the instructions.

The various methods or processes outlined herein may be coded as software that is executable on one or more processors that employ any one of a variety of operating systems or platforms. The disclosure also relates to a computer readable storage medium comprising executable instructions. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention disclosed herein. The computer readable medium or media can be transportable, such that the program or programs stored thereon can be loaded onto one or more different computers or other processors to implement various aspects of the present invention as discussed above. In some embodiments, the system comprises cloud-based software that executes one or all of the steps of each disclosed method instruction.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present disclosure need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, the disclosure relates to various embodiments in which one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Computer-implemented embodiments of the disclosure relate to methods of determining a subject likely to respond to disease-modifying agents comprising steps of: (e) comparing the first normalized score to a first threshold relative to a first control dataset of a sample and comparing a second normalized score to a second threshold relative to a control dataset of the sample; and (f) classifying the subject as being likely to respond to a chemotherapeutic treatment based upon results of comparing of step (e) relative to the first and/or second threshold; wherein each of steps (e) and (f) are performed after step (d).

In some embodiments, the disclosure relates to a system that comprises at least one processor, a program storage, such as memory, for storing program code executable on the processor, and one or more input/output devices and/or interfaces, such as data communication and/or peripheral devices and/or interfaces. In some embodiments, the user device and computer system or systems are communicably connected by a data communication network, such as a Local Area Network (LAN), the Internet, or the like, which may also be connected to a number of other client and/or server computer systems. The user device and client and/or server computer systems may further include appropriate operating system software.

In some embodiments, components and/or units of the devices described herein may be able to interact through one or more communication channels or mediums or links, for example, a shared access medium, a global communication network, the Internet, the World Wide Web, a wired network, a wireless network, a combination of one or more wired networks and/or one or more wireless networks, one or more communication networks, an a-synchronic or asynchronous wireless network, a synchronic wireless network, a managed wireless network, a non-managed wireless network, a burstable wireless network, a non-burstable wireless network, a scheduled wireless network, a non-scheduled wireless network, or the like.

Discussions herein utilizing terms such as, for example, “processing,” “computing,” “calculating,” “determining,” or the like, may refer to operation(s) and/or process(es) of a computer, a computing platform, a computing system, or other electronic computing device, that manipulate and/or transform data represented as physical (e.g., electronic) quantities within the computer's registers and/or memories into other data similarly represented as physical quantities within the computer's registers and/or memories or other information storage medium that may store instructions to perform operations and/or processes.

Some embodiments may take the form of an entirely hardware embodiment, an entirely software embodiment, or an embodiment including both hardware and software elements. Some embodiments may be implemented in software, which includes but is not limited to firmware, resident software, microcode, or the like.

Furthermore, some embodiments may take the form of a computer program product accessible from a computer-usable or computer-readable medium providing program code for use by or in connection with a computer or any instruction execution system. For example, a computer-usable or computer-readable medium may be or may include any apparatus that can contain, store, communicate, propagate, or transport the program for use by or in connection with the instruction execution system, apparatus, or device.

In some embodiments, the medium may be or may include an electronic, magnetic, optical, electromagnetic, InfraRed (IR), or semiconductor system (or apparatus or device) or a propagation medium. Some demonstrative examples of a computer-readable medium may include a semiconductor or solid state memory, magnetic tape, a removable computer diskette, a Random Access Memory (RAM), a Read-Only Memory (ROM), a rigid magnetic disk, an optical disk, or the like. Some demonstrative examples of optical disks include Compact Disk-Read-Only Memory (CD-ROM), Compact Disk-Read/Write (CD-R/W), DVD, or the like.

In some embodiments, a data processing system suitable for storing and/or executing program code may include at least one processor coupled directly or indirectly to memory elements, for example, through a system bus. The memory elements may include, for example, local memory employed during actual execution of the program code, bulk storage, and cache memories which may provide temporary storage of at least some program code in order to reduce the number of times code must be retrieved from bulk storage during execution.

In some embodiments, input/output or I/O devices (including but not limited to keyboards, displays, pointing devices, etc.) may be coupled to the system either directly or through intervening I/O controllers. In some embodiments, network adapters may be coupled to the system to enable the data processing system to become coupled to other data processing systems or remote printers or storage devices, for example, through intervening private or public networks. In some embodiments, modems, cable modems and Ethernet cards are demonstrative examples of types of network adapters. Other suitable components may be used.

Some embodiments may be implemented by software, by hardware, or by any combination of software and/or hardware as may be suitable for specific applications or in accordance with specific design requirements. Some embodiments may include units and/or sub-units, which may be separate of each other or combined together, in whole or in part, and may be implemented using specific, multi-purpose or general processors or controllers. Some embodiments may include buffers, registers, stacks, storage units and/or memory units, for temporary or long-term storage of data or in order to facilitate the operation of particular implementations.

Some embodiments may be implemented, for example, using a machine-readable medium or article which may store an instruction or a set of instructions that, if executed by a machine, cause the machine to perform a method steps and/or operations described herein. Such machine may include, for example, any suitable processing platform, computing platform, computing device, processing device, electronic device, electronic system, computing system, processing system, computer, processor, or the like, and may be implemented using any suitable combination of hardware and/or software. The machine-readable medium or article may include, for example, any suitable type of memory unit, memory device, memory article, memory medium, storage device, storage article, storage medium and/or storage unit; for example, memory, removable or non-removable media, erasable or non-erasable media, writeable or re-writeable media, digital or analog media, hard disk drive, floppy disk, Compact Disk Read Only Memory (CD-ROM), Compact Disk Recordable (CD-R), Compact Disk Re-Writeable (CD-RW), optical disk, magnetic media, various types of Digital Versatile Disks (DVDs), a tape, a cassette, or the like. The instructions may include any suitable type of code, for example, source code, compiled code, interpreted code, executable code, static code, dynamic code, or the like, and may be implemented using any suitable high-level, low-level, object-oriented, visual, compiled and/or interpreted programming language, e.g., C, C++, Java™, BASIC, Pascal, Fortran, Cobol, assembly language, machine code, or the like.

Many of the functional units described in this specification have been labeled as circuits, in order to more particularly emphasize their implementation independence. For example, a circuit may be implemented as a hardware circuit comprising custom very-large-scale integration (VLSI) circuits or gate arrays, off-the-shelf semiconductors such as logic chips, transistors, or other discrete components. A circuit may also be implemented in programmable hardware devices such as field programmable gate arrays, programmable array logic, programmable logic devices or the like.

In some embodiment, the circuits may also be implemented in machine-readable medium for execution by various types of processors. An identified circuit of executable code may, for instance, comprise one or more physical or logical blocks of computer instructions, which may, for instance, be organized as an object, procedure, or function. Nevertheless, the executables of an identified circuit need not be physically located together, but may comprise disparate instructions stored in different locations which, when joined logically together, comprise the circuit and achieve the stated purpose for the circuit. Indeed, a circuit of computer readable program code may be a single instruction, or many instructions, and may even be distributed over several different code segments, among different programs, and across several memory devices. Similarly, operational data may be identified and illustrated herein within circuits, and may be embodied in any suitable form and organized within any suitable type of data structure. The operational data may be collected as a single data set, or may be distributed over different locations including over different storage devices, and may exist, at least partially, merely as electronic signals on a system or network.

The computer readable medium (also referred to herein as machine-readable media or machine-readable content) may be a tangible computer readable storage medium storing the computer readable program code. The computer readable storage medium may be, for example, but not limited to, an electronic, magnetic, optical, electromagnetic, infrared, holographic, micromechanical, or semiconductor system, apparatus, or device, or any suitable combination of the foregoing. As alluded to above, examples of the computer readable storage medium may include but are not limited to a portable computer diskette, a hard disk, a random access memory (RAM), a read-only memory (ROM), an erasable programmable read-only memory (EPROM or Flash memory), a portable compact disc read-only memory (CD-ROM), a digital versatile disc (DVD), an optical storage device, a magnetic storage device, a holographic storage medium, a micromechanical storage device, or any suitable combination of the foregoing. In the context of this document, a computer readable storage medium may be any tangible medium that can contain, and/or store computer readable program code for use by and/or in connection with an instruction execution system, apparatus, or device.

The computer readable medium may also be a computer readable signal medium. A computer readable signal medium may include a propagated data signal with computer readable program code embodied therein, for example, in baseband or as part of a carrier wave. Such a propagated signal may take any of a variety of forms, including, but not limited to, electrical, electro-magnetic, magnetic, optical, or any suitable combination thereof. A computer readable signal medium may be any computer readable medium that is not a computer readable storage medium and that can communicate, propagate, or transport computer readable program code for use by or in connection with an instruction execution system, apparatus, or device. As also alluded to above, computer readable program code embodied on a computer readable signal medium may be transmitted using any appropriate medium, including but not limited to wireless, wireline, optical fiber cable, Radio Frequency (RF), or the like, or any suitable combination of the foregoing. In one embodiment, the computer readable medium may comprise a combination of one or more computer readable storage mediums and one or more computer readable signal mediums. For example, computer readable program code may be both propagated as an electro-magnetic signal through a fiber optic cable for execution by a processor and stored on RAM storage device for execution by the processor.

Computer readable program code for carrying out operations for aspects of the present disclosure may be written in any combination of one or more programming languages, including an object oriented programming language such as Java, Smalltalk, C++ or the like and conventional procedural programming languages, such as the “C” programming language or similar programming languages. The computer readable program code may execute entirely on a user's computer, partly on the user's computer, as a stand-alone computer-readable package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The program code may also be stored in a computer readable medium that can direct a computer, other programmable data processing apparatus, or other devices to function in a particular manner, such that the instructions stored in the computer readable medium produce an article of manufacture including instructions which implement the function/act specified in the schematic flowchart diagrams and/or schematic block diagrams block or blocks.

Functions, operations, components and/or features described herein with reference to one or more embodiments, may be combined with, or may be utilized in combination with, one or more other functions, operations, components and/or features described herein with reference to one or more other embodiments, or vice versa.

Although the disclosure has been described with reference to exemplary embodiments, it is not limited thereto. Those skilled in the art will appreciate that numerous changes and modifications may be made to the preferred embodiments of the disclosure and that such changes and modifications may be made without departing from the true spirit of the disclosure. It is therefore intended that the appended claims be construed to cover all such equivalent variations as fall within the true spirit and scope of the disclosure.

All referenced journal articles, patents, and other publications are incorporated by reference herein in their entireties.

### Cryo-EM

Cryogenic electron microscopy, also known as electron cryomicroscopy (cryo-EM), is an electron microscopy (EM) technique applied on samples cooled to cryogenic temperatures and embedded in an environment of vitreous water. Cryo-EM is an emerging, computer vision-based approach to determine 3-dimensional (3D) macromolecular structure with subnanometre resolution. Cryo-EM is applicable to medium to large-sized molecules in their native state. This scope of applicability is in contrast to X-ray crystallography, which requires a crystal of the target molecule, which are often impossible to grow, or nuclear magnetic resonance (NMR) spectroscopy, which is limited to relatively small molecules. Cryo-EM has the potential to unveil the molecular and chemical nature of fundamental biology through the discovery of atomic structures of previously unknown biological structures, many of which have proven difficult or impossible to study by conventional structural biology techniques.

In cryo-EM, molecules are embedded in a frozen-hydrated state, suspended across holes in a thin carbon film (R. Henderson, Q. Rev. Biophys. 37, 3 (2004); and W. Chiu et al, Structure 13, 363 (2005)), and then imaged with a transmission electron microscope in the presence of coherent, high-energy electrons (10-50 eVA2). A large number of such samples are obtained, each of which provides a micrograph containing hundreds of visible, individual molecules. In a process known as particle picking, individual molecules are imaged, resulting in a stack of cropped images of the molecule (referred to as particle images). Each particle image provides a noisy view of the molecule with an unknown pose. Once a large set of 2-dimensional (2D) electron microscope particle images of the molecule have been obtained, reconstruction is carried out to estimate the 3D density of a target molecule from the images. The ability of cryo-EM to resolve the structures of complex proteins depends on the techniques underlying the reconstruction process.

Generally, images obtained by cryo-EM can be analyzed to identify micrographs of single particles. Single particle selection can be done with the help of software tools such as SIGNATURE (Chen & Grigorieff (2007) J Struct Biol 157(1):168-73). The astigmatic defocus, specimen tilt axis, and tilt angle for each micrograph can be determined using the computer program CTFTILT (Mindell & Grigorieff (2003) J Struct Biol 142(3):334-47). Obtaining separate defocus values for each particle according to its coordinate in the original image improves the data quality of the cryo-EM density map which is obtained by averaging single-particle micrographs of particles.

Fitting of known atomic models within a cryo-EM density map is a common approach for building models of complex structures. A number of computational fitting tools are available which range from simple rigid-body localization of protein structures, such as Situs (Wriggers et al. (1999) J Struct Biol 125(2-3):185-95), Foldhunter (Jiang et al. (2001) J Mol Biol 308(5):1033-44) and Mod-EM (Topf et al. (2005) J Struct Biol 149(2):191-203), to complex and dynamic flexible fitting algorithms like NMFF (Tama et al. (2004) J Struct Biol 147(3):315-2), Flex-EM (Topf et al. (2008) Structure 16(2):295-307), MDFF (Trabuco et al. (2009) Methods 49(2):174-80) and DireX (Schroder et al. (2007) Structure 15(12):1630-41; Zhang et al. (2010) Nature 463(7279):379-83), which morph known structures to a density map.

When an atomic model is not known, cryo-EM density maps can be used in building and/or evaluating structural models from a gallery of potential models that are constructed in silico (see Topf et al. (2005) J Struct Biol 149(2):191-203; Baker et al. (2006) PLoS Comput Biol 2(10):e146; DiMaio et al. (2009) J Mol Biol 392(1):181-90; Topf et al. (2006) J Mol Biol 357(5):1655-68; Zhu et al. (2010) J Mol Biol 397(3):835-51). A related template structure must be known for constrained comparative modeling or, for constrained ab initio modeling, the fold to be modelled must be relatively small. For example, an initial structure may be obtained using IMIRS (Liang et al. (2002) J Struct Biol 137(3):292-304). Further alignment and reconstruction can be performed with FREALIGN (Grigorieff (2007) J Struct Biol 157(1):117-25) using a known protein structure and a known structure of a heterologous protein or a close homologue as template.

Significant structural and functional information can be obtained directly from the density map itself. For example, at from about 5 to about 10 Å resolutions, some secondary structure elements are visible in cryo-EM density maps: α-helices appear as cylinders, while β-sheets appear as thin, curved plates. These secondary structure elements can be reliably identified and quantified using feature recognition tools to describe a protein structure or infer the function of individual proteins. At near-atomic resolutions (3-5 Å), the pitch of α-helices, separation of β-strands, as well as the densities that connect them, can be visualized unambiguously (see e.g., Cheng et al. (2010) J Mol Biol 397(3):852-63; Jiang et al. (2008) Nature 451(7182):1130-4; Ludtke et al. (2008) Structure 16(3):441-8; Yu et al. (2008) Nature 453(7193):415-9). The disclosure relates to a method of creating a cryo-EM image or performing cryo-EM imaging comprising:
(a) calculating a differential interaction score (DIS); (b) applying the DIS score a density map readable by one or more computer program products capable of displaying an image corresponding to the readable density map; (c) displaying an image of a protein on a display in operable communication with a controller or system comprising the computer program product. In some embodiments, the method further comprises (d) correlating the DIS with the likelihood that a dysfunctional protein-protein interaction is the causal agent of the disorder. In some embodiments, the resulting image of the method of performing cryo-EM has a resolution from about 5 to about 20 angstroms, from about 5 to about 15 angstroms, or from about 5 to about 10 angstroms. In some embodiments, the image created by applying the DIS score has a resolution of about 5, about 6, about 7, about 8, about 9, about 10, about 11, about 12, about 13, about 14, about 15, about 16, about 17, about 18, about 19 or about 20 angstroms.

De novo model building in cryo-EM comprises feature recognition, sequence analysis, secondary structure element correspondence, Ca placement and model optimization. Various software applications can be used, e.g., EMAN for density map segmentation and manipulation (Ludtke et al. (1999) J Struct Biol 128(1):82-97), SSEHunter (Baker et al. (2007) Structure 15(1):7-19) to detect secondary structure elements, visualization in UCSF's Chimera (Pettersen et al. (2004) J Comput Chem 25(13):1605-12) and atom manipulation in Coot (Emsley & Cowtan (2004) Acta Crystallogr D Biol Crystallogr 60(Pt 12 Pt 1):2126-32; Emsley et al. (2010) Acta Crystallogr D Biol Crystallogr 66(Pt 4):486-501).

Secondary structure identification programs like SSEHunter provide a semi-automated mechanism for detecting and displaying visually observable secondary structure elements in a density map (Baker et al. (2007) Structure 15(1):7-19). Registration of secondary structure elements in the sequence and structure, combined with geometric and biophysical information, can be used to anchor the protein backbone in the density map (Cheng et al. (2010) J Mol Biol 397(3):852-63; Ludtke et al. (2008) Structure 16(3):441-8). This sequence-to-structure correspondence relates the observed secondary structure elements in the density to those predicted in the sequence. The modeling toolkit GORGON couples sequence-based secondary structure prediction with feature detection and geometric modeling techniques to generate initial protein backbone models (Baker et al. (2011) J Struct Biol 174(2):360-73). Automatic modeling methods such as EM-IMO (electron microscopy-iterative modular optimization) can be used for building, modifying and refining local structures of protein models using cryo-EM maps as a constraint (Zhu et al. (2010) J Mol Biol 397(3):835-51).

Once a correspondence has been determined using secondary structure element, Ca atoms can be assigned to the density beginning with α-helices and followed by β-strands and loops. For example, by taking advantage of clear bumps for Ca atoms, Ca models can be built using the Baton build utility in the crystallographic programs 0 (Jones et al. (1991) Acta Cystallogr A 47 (Pt 2):110-9) and/or Coot (Emsley & Cowtan (2004) Acta Crystallogr D Biol Crystallogr 60(Pt 12 Pt 1):2126-32). Ca positions can be interactively adjusted such that they fit the density optimally while maintaining reasonable geometries and eliminating clashes within the model. Coarse full-atom models can be refined in a pseudocrystallographic manner using CNS (Brunger et al. (1998) Acta Cystallogr D Biol Crystallogr 54(Pt 5):905-21). Models can be further optimized using computational modeling software such as Rosetta (DiMaio et al. (2009) J Mol Biol 392(1):181-90). Full-atom models can also be built with the help of other computational tools such as REMO (Li & Zhang (2009) Proteins 76(3):665-76). The quality of a model can be confirmed by visual comparison of the model with the density map. Pseudocrystallographic R factor/Rfree analysis (Briinger (1992) Nature 355(6359):472-5) provides a measure of the agreement between observed and computed structure factor amplitudes and may be used to confirm that the obtained atomic model provides a good fit to the cryo-EM density maps. Protein model geometry can be checked by PROCHECK (Laskowski et al. (1993) J Appl Cryst 26:283-91).

In cryo-EM, the image intensity is a reflection of the electron phase shift due to electrostatic potentials, including the internal potentials of the atoms in the specimen. In the weak-phase approximation, the Fourier transform I(s) of the image intensity I(x,y) is most readily expressed in terms of the two-dimensional spatial frequency s, as:

Î(s)=Î₀[δ(s)+2h(s)\hat{φ}(s)]

In the equation above, Î₀ is the mean image intensity, δ(s) is the two dimensional Dirac delta function, and h(s) is the contrast transfer function (CTF). T\hat{φ}(s)nction is the Fourier transform of the specimen's phase shift φ(x, y). The image contrast depends on a number of factors including the ice thickness, as unstained biological specimens are embedded in a thin film (e.g., ˜100 nm) of vitreous ice:

$$C = \frac{Δ I}{{I}_{s}} = \frac{( {φ}_{protein} - {φ}_{water} ) \cdot {t}_{protein}}{{φ}_{water} \cdot {t}_{ice}}$$

In the equation above, φₚᵣₒₜₑᵢₙ and φ_{water} are phase shifts of electrons passing through protein and water regions, and tₚᵣₒₜₑᵢₙ and t_{ice} are thicknesses of the protein molecules and ice layer, respectively. The calculated image contrast drops dramatically as the ice thickness increases from, e.g., 10 nm to 100 nm. The protein particles may be clearly seen when contained in a thin ice layer, but not in a thick ice layer. Experiments have shown that by extensive efforts to optimize the vitrification process, the contrast of recorded cryo-EM images may increase dramatically.

Resolution

While cryo-EM could be used as a substitute technique for protein crystallography, the main drawback, however, is the low resolution of the structures obtainable with conventional technology. For example, resolutions of about 7.4 Å (angstroms) have been achieved for virus analysis and resolutions of about 11.5 Å have been achieved for large protein complexes such as ribosome. With recent improvement in this technology, cryo-EM resolutions are now approaching 1.5 ångströms (Å) (Bhella, D., Biophysical Reviews. 2019, 11 (4): 515-519).

In some embodiments, the resolution of the structures obtainable with the methods of the disclosure is from about 1.0 Å to about 20.0 Å. In some embodiments, the resolution of the structures obtainable with the methods of the disclosure is from about 2.0 Å to about 18.0 Å. In some embodiments, the resolution of the structures obtainable with the methods of the disclosure is from about 2.5 Å to about 16.0 Å. In some embodiments, the resolution of the structures obtainable with the methods of the disclosure is from about 3.0 Å to about 14.0 Å. In some embodiments, the resolution of the structures obtainable with the methods of the disclosure is from about 3.5 Å to about 12.0 Å. In some embodiments, the resolution of the structures obtainable with the methods of the disclosure is from about 4.0 Å to about 10.0 Å. In some embodiments, the resolution of the structures obtainable with the methods of the disclosure is from about 4.5 Å to about 8.0 Å.

In some embodiments, the resolution of the structures obtainable with the methods of the disclosure is about 1.0 Å. In some embodiments, the resolution of the structures obtainable with the methods of the disclosure is about 1.5 Å. In some embodiments, the resolution of the structures obtainable with the methods of the disclosure is about 2.0 Å. In some embodiments, the resolution of the structures obtainable with the methods of the disclosure is about 2.5 Å. In some embodiments, the resolution of the structures obtainable with the methods of the disclosure is about 3.0 Å. In some embodiments, the resolution of the structures obtainable with the methods of the disclosure is about 3.5 Å. In some embodiments, the resolution of the structures obtainable with the methods of the disclosure is about 4.0 Å. In some embodiments, the resolution of the structures obtainable with the methods of the disclosure is about 4.5 Å. In some embodiments, the resolution of the structures obtainable with the methods of the disclosure is about 5.0 Å. In some embodiments, the resolution of the structures obtainable with the methods of the disclosure is about 5.5 Å. In some embodiments, the resolution of the structures obtainable with the methods of the disclosure is about 6.0 Å. In some embodiments, the resolution of the structures obtainable with the methods of the disclosure is about 6.5 Å. In some embodiments, the resolution of the structures obtainable with the methods of the disclosure is about 7.0 Å. In some embodiments, the resolution of the structures obtainable with the methods of the disclosure is about 7.5 Å. In some embodiments, the resolution of the structures obtainable with the methods of the disclosure is about 8.0 Å. In some embodiments, the resolution of the structures obtainable with the methods of the disclosure is about 8.5 Å. In some embodiments, the resolution of the structures obtainable with the methods of the disclosure is about 9.0 Å. In some embodiments, the resolution of the structures obtainable with the methods of the disclosure is about 9.5 Å. In some embodiments, the resolution of the structures obtainable with the methods of the disclosure is about 10.0 Å. In some embodiments, the resolution of the structures obtainable with the methods of the disclosure is about 11.0 Å. In some embodiments, the resolution of the structures obtainable with the methods of the disclosure is about 12.0 Å. In some embodiments, the resolution of the structures obtainable with the methods of the disclosure is about 13.0 Å. In some embodiments, the resolution of the structures obtainable with the methods of the disclosure is about 14.0 Å. In some embodiments, the resolution of the structures obtainable with the methods of the disclosure is about 15.0 Å. In some embodiments, the resolution of the structures obtainable with the methods of the disclosure is about 16.0 Å. In some embodiments, the resolution of the structures obtainable with the methods of the disclosure is about 17.0 Å. In some embodiments, the resolution of the structures obtainable with the methods of the disclosure is about 18.0 Å. In some embodiments, the resolution of the structures obtainable with the methods of the disclosure is about 19.0 Å. In some embodiments, the resolution of the structures obtainable with the methods of the disclosure is about 20.0 Å.

Methods

The disclosure further relates to methods of predicting three-dimensional (3D) structure of macromolecules, such as proteins, protein complexes, and viral particles, by combining structural-biology techniques and artificial-intelligence (AI) techniques. The traditional structural-biology techniques, such as nuclear magnetic resonance (NMR) spectroscopy, X-ray crystallography, and cryo-electron microscopy (cryo-EM), predict the 3D structure of a macromolecule based on the molecule itself. The AI techniques, based on machine deep learning, predict the 3D structure of a macromolecule based on genomic data.

Artificial-Intelligence (AI) Techniques

The AI techniques computationally predict the 3D structure of a macromolecule based solely on genomic data. These techniques generally involve use of deep neural networks to predict protein structure based on sequence. Several algorithms have been developed for such prediction.

AlphaFold, for example, is such an algorithm developed by DeepMind (London, UK) that focuses specifically on the problem of modeling target shapes from scratch, without using previously solved proteins as templates. AlphaFold can achieve a high degree of accuracy when predicting the physical properties of a protein structure, and then used two distinct methods to construct predictions of full protein structures. Both of these methods rely on deep neural networks that are trained to predict properties of the protein from its genetic sequence. The properties AlphaFold's networks predict are: (a) the distances between pairs of amino acids and (b) the angles between chemical bonds that connect those amino acids.

AlphaFold works in two steps. It starts with so-called multiple sequence alignments by comparing a protein's sequence with similar ones in a database to reveal pairs of amino acids that do not lie next to each other in a chain, but that tend to appear in tandem. This suggests that these two amino acids are located near each other in the folded protein. AlphaFold trains a neural network to take such pairings to predict a distribution of distances between every pair of residues in a folded protein. These probabilities are then combined into a score that estimates how accurate a proposed protein structure is. By comparing its predictions with precisely measured distances in proteins, AlphaFold learns to make better guesses about how proteins would fold up. In parallel, AlphaFold also trains another neural network predicting the angles of the joints between consecutive amino acids in the folded protein chain.

Using these scoring functions, AlphaFold is able to search the protein landscape to find structures that match the predictions. The first method used in AlphaFold is built on techniques commonly used in structural biology, and repeatedly replaced pieces of a protein structure with new protein fragments. AlphaFold trains a generative neural network to invent new fragments, which were used to continually improve the score of the proposed protein structure.

In a second step, AlphaFold creates a physically possible—but nearly random—folding arrangement for a sequence. Instead of using another neural network, AlphaFold uses an optimization method called gradient descent—a mathematical technique commonly used in machine learning for making small, incremental improvements—to optimize scores and iteratively refine the structure so it comes close to the (not-quite-possible) predictions from the first step and results in highly accurate structures. This technique is applied to entire protein chains rather than to pieces that must be folded separately before being assembled into a larger structure, to simplify the prediction process.

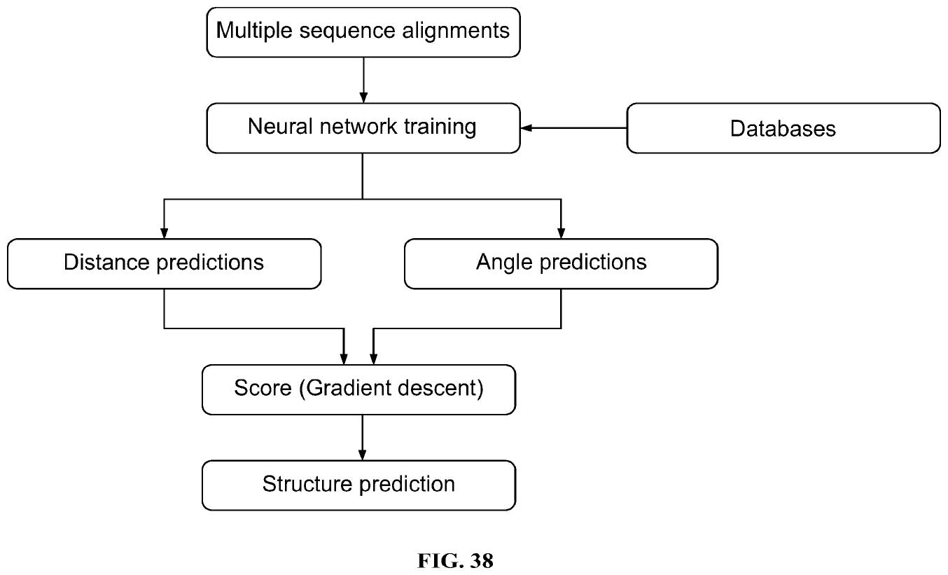

A representative flowchart illustrating the architecture of the Alphafold system for predicting structure from protein sequence is provided in FIG. 38.

Another algorithm for protein 3D structure prediction was developed by Mohammed AlQuraishi, a biologist at Harvard Medical School in Boston, Massachusetts. This algorithm uses a totally different approach. Instead of 2-step approaches as AlphaFold, AlQuraishi's algorithm uses a mathematical function to calculate protein structures in a single step. At the core of AlQuraishi's approach is again a neural network that is fed with known data on how amino-acid sequences map to protein structures and then learns to produce new structures from unfamiliar sequences. Instead of using a neural network to predict certain features of a structure, such as the neural networks predicting the angles and distances between amino acids in the folded protein used in AlphaFold, AlQuraishi's system uses end-to-end differentiable deep learning to create mappings end-to-end and then use an algorithm to laboriously search for a plausible structure that incorporates those features. This approach, which AlQuraishi dubs a recurrent geometric network, predicts the structure of one segment of a protein partly on the basis of what comes before and after it. AlQuraishi's algorithm is published in AlQuraishi, Cell Systems, 2019, 8: 292-301, incorporated by reference herein.

AlQuraishi's model featurizes a protein of length L as a sequence of vectors (x₁, . . . , X_{L}) where xₜ∈R^{d} for all t. The dimensionality d is 41, where 20 dimensions are used as a one-hot indicator of the amino acid residue at a given position, another 20 dimensions are used for the PSSM of that position, and 1 dimension is used to encode the information content of the position. The PSSM values are sigmoid transformed to lie between 0 and 1. The sequence of input vectors are fed to an LS™ (Hochreiter and Schmidhuber, Neural Comput., 1997, 9(8):1735-1780), whose basic formulation is described by the following set of equation.

iₜ=σ(Wᵢ[xₜ,hₜ₋₁]+bᵢ),

fₜ=σ(W_{f}[xₜ,hₜ₋₁]+b_{f}),

oₜ=σ(Wₒ[xₜ,hₜ₋₁]+bₒ),

\tilde{c}ₜ=tan h(W_{c}[xₜ,hₜ₋₁]+b_{c}),

cₜ=iₜ⊙\tilde{c}ₜ+fₜ⊙cₜ₋₁,

hₜ=oₜ⊙ tan h(cₜ),

Wᵢ, W_{f}, Wₒ, W_{c} are weight matrices, bᵢ, b_{f}, bₒ, b_{c} are bias vectors, hₜ and cₜ are the hidden and memory cell state for residue t, respectively, and Θ is element-wise multiplication. It uses two LSTMs, running independently in opposite directions (1 to L and L to 1), to output two hidden states hₜ^{(f)} and hₜ^{(b)} for each residue position t corresponding to the forward and backward directions. Depending on the RGN architecture, these two hidden states are either the final outputs states or they are fed as inputs into one or more LS™ layers.

The outputs from the last LSTM layer form a sequence of a concatenated hidden state vectors ([h_{I}^{(f)}, h_{I}^{(b)}], . . . , [h_{L}^{(f)}, h_{L}^{(b)}]). Each concatenated vector is then fed into an angularization layer described by the following set of equations:

pₜ=softmax(W_{φ}[hₜ^{(f)},hₜ^{(b)}]+b_{φ}).

φₜ=arg(pₜexo(iΦ)).

W_{φ} is a weight matrix, bφ is a bias vector, Φ is a learned alphabet matrix, and arg is the complex-valued argument function. Exponentiation of the complex-valued matrix iΦ is performed element-wise. The Φ matrix defines an alphabet of size m whose letters correspond to triplets of torsional angles defined over the 3-torus. The angularization layer interprets the LS™ hidden state outputs as weights over the alphabet, using them to compute a weighted average of the letters of the alphabet (independently for each torsional angle) to generate the final set of torsional angles φₜ∈S^{I}×S^{I}×S^{I} for residue t (the standard notation for protein backbone torsional angles are overloaded, with φt corresponding to the (ψ, φ, ω) triplet). Note that φt may be alternatively computed using the following equation, where the trigonometric operations are performed element-wise:

φₜ=a tan 2(pₜ sin(Φ),pₜ cos(Φ)).

In general, the geometry of a protein backbone can be represented by three torsional angles φ, ψ, and ω that define the angles between successive planes spanned by the N, C^{α}, and C′ protein backbone atoms (Ramachandran et al., J. Mol. Biol., 1963, 7:95-99). While bond lengths and angles vary as well, their variation is sufficiently limited that they can be assumed fixed. Similar claims hold for side chains as well, although the attention is restricted to backbone structure. The resulting sequence of torsional angles (φ₁, . . . , φ_{L}) from the angularization layer is fed sequentially, along with the coordinates of the last three atoms of the nascent protein chain (c₁, c₃ₜ), into recurrent geometric units that convert this sequence into 3D Cartesian coordinates, with three coordinates resulting from each residue, corresponding to the N, Cα, and C′ backbone atoms. Multiple mathematically-equivalent formulations exist for this transformation; one is adopted based on the Natural Extension Reference Frame (Parsons et al., J. Comput. Chem., 2005, 26(10):1063-1068.), described by the following set of equations:

$${\hat{c}}_{k} = {f}_{kmod 3} [ \begin{matrix}cos ( {θ}_{kmod 3} ) \\ cos ( {φ}_{k / 3 kmod 3} ) sin ( {θ}_{kmod 3} ) \\ sin ( {φ}_{k / 3 kmod 3} ) sin ( {θ}_{kmod 3} )\end{matrix} ] , \\ {m}_{k} = {c}_{k - 1} - {c}_{k - 2} , \\ {n}_{k} = {m}_{k - 1} \times \hat{{m}_{k}} , \\ {M}_{k} = [ \hat{{m}_{k}} , \hat{{n}_{k}} \times \hat{{m}_{k}} , \hat{{n}_{k}} ] , \\ {c}_{k} = {M}_{k} \hat{{c}_{k}} + {c}_{k - 1} .$$

Where rₖ is the length of the bond connecting atoms k−1 and K, θₖ is the bond angle formed by atoms k−2, k−1, and k, φ_{k/3,k mod 3} is the predicted torsional angle formed by atoms k−2 and k−1, Cₖ is the position of the newly predicted atom k, \hat{m} is the unit-normalized version of m, and x is the cross product. Note that k indexes atoms 1 through 3 L, since there are three backbone atoms per residue. For each residue t, it is computed C₃ₜ₋₂, C₃ₜ₋₁, and C₃ₜ using the three predicted torsional angles of residue t, specifically

$${φ}_{t , j} = {φ}_{⌊ \frac{3 t}{3} ⌋ , ( 3 t + j ) mod 3}$$

for j={0,1,2}. The bond lengths and angles are fixed, with three bond length (r₀, r₁, r₂) corresponding to N—C^{α}, C^{α}—C′, and C′—N, and three bond angles (θ₀, θ₁, θ₂) corresponding to N—C^{α}—C′, C^{α}—C′—N, and C′—N—C^{α}. As there are only three unique values we have rₖ=r_{k mod 3} and θ₆ₖ=θ_{k mod 3}. In practice, a modified version of the above equations which enable much higher computational efficiency is employed (AlQuraishi, J. Comput. Chem., 2019, 40(7):885-892).

The resulting sequence (C₁, . . . , C_{3L}) fully describes the protein backbone chain structure and is the model's final predicted output. For training purposes a loss is necessary to optimize model parameters. The dRMSD metric is used as it is differentiable and captures both local and global aspects of protein structure. It is defined by the following set of equations:

$${\tilde{d}}_{j , k} = {‖ {c}_{j} - {c}_{k} ‖}_{2} . \\ {d}_{j , k} = {\tilde{d}}_{j , k}^{( exp )} - {\tilde{d}}_{j , k}^{( pred )} . \\ dRMSD = \frac{{‖ D ‖}_{2}}{L ( L - 1 )} .$$

where {dj,k} are the elements of matrix D, and \tilde{d}_{j,k}^{-(exp)} and \tilde{d}_{j,k}^{(pred)} are computed using the coordinates of the experimental and predicted structures, respectively. In effect, the dRMSD computes the l2-norm of the distances over distances, by first computing the pairwise distances between all atoms in both the predicted and experimental structures individually, and then computing the distances between those distances. For most experimental structures, the coordinates of some atoms are missing. They are excluded from the dRMSD by not computing the differences between their distances and the predicted ones.

RGN hyperparameters were manually fit, through sequential exploration of hyperparameter space, using repeated evaluations on the ProteinNet11 validation set and three evaluations on ProteinNet11 test set. Once chosen the same hyperparameters were used to train RGNs on ProteinNet7-12 training sets. The validation sets were used to determine early stopping criteria, followed by single evaluations on the ProteinNet7-12 test sets to generate the final reported numbers (excepting ProteinNet11).

The final model consisted of two bidirectional LSTM layers, each comprised of 800 units per direction, and in which outputs from the two directions are first concatenated before being fed to the second layer. Input dropout set at 0.5 was used for both layers, and the alphabet size was set to 60 for the angularization layer. Inputs were duplicated and concatenated; this had a separate effect from decreasing dropout probability. LSTMs were random initialized with a uniform distribution with support [−0.001, 0.01], while the alphabet was similarly initialized with support [−π, π]. ADAM was used as the optimizer, with a learning rate of 0.001, β1=0.95 and β2=0.99, and a batch size of 32. Gradients were clipped using norm rescaling with a threshold of 5.0. The loss function used for optimization was length-normalized dRMSD (i.e. dRMSD divided by protein length), which is distinct from the standard dRMSD used for reporting accuracies.

RGNs are very seed sensitive. As a result, a milestone scheme is used to restart underperforming models early. If a dRMSD loss milestone is not achieved by a given iteration, training is restarted with a new initialization seed. In general, 8 models were started and, after surviving all milestones, were run for 250 k iterations, at which point the lower performing half were discarded, and similarly at 500 k iterations, ending with 2 models that were usually run for ˜2.5M iterations. Once validation error stabilized, the learning rate is reduced by a factor of 10 to 0.0001, and run for a few thousand additional iterations to gain a small but detectable increase in accuracy before ending model training.

Determination of 3-Dimensional Structure of a Protein of Interest

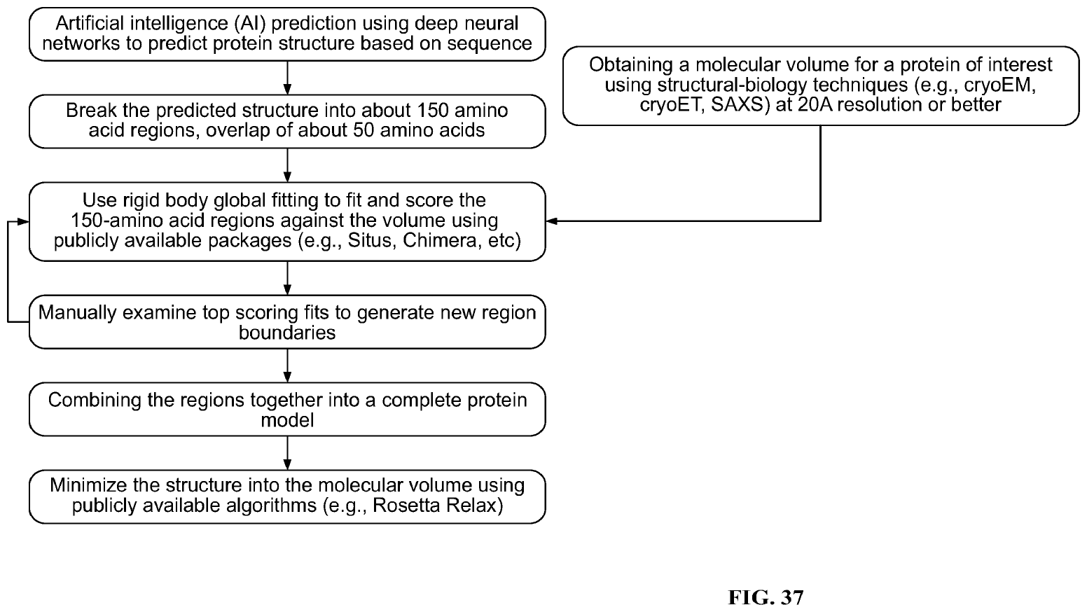

Referring to FIG. 37, which shows a representative flowchart illustrating the use of structural-biology techniques in combination with artificial intelligence (AI) prediction to construct a 3-dimensional (3D) structure of a protein. Based on this flowchart, the methods of the disclosure comprises the following steps: (a) obtaining a molecular volume for a protein of interest using a structural-biology technique at a resolution of about 20 Å or better; (b) predicting a 3D structure of the protein of interest based on artificial intelligence (AI) prediction using one or a plurality of deep neural networks to predict the 3D structure based on sequence; (c) breaking the 3D structure predicted in step (b) into overlapping regions; (d) global rigid-body fitting the overlapping regions against the molecular volume obtained in step (a); (e) examining top scoring fits and generating new region boundaries; (f) optionally repeating steps (d) and (e) for one or a plurality of times; (g) combining the regions into a complete protein structure; and (h) refining the complete protein structure obtained in step (g) into the molecular volume of (a).

In some embodiments, the structural-biology technique used in the methods of the disclosure comprises cryo-EM. In some embodiments, the structural-biology technique used in the methods of the disclosure comprises cryo-TM. In some embodiments, the structural-biology technique used in the methods of the disclosure comprises small angle x-ray scattering (SAXS).

In some embodiments, the resolution of the molecular volume of the protein of interest obtained by the structural-biology technique used in the methods of the disclosure is from about 4 Å to about 10 Å. In some embodiments, the resolution is from about 5 Å to about 11 Å. In some embodiments, the resolution is from about 6 Å to about 12 Å. In some embodiments, the resolution is from about 7 Å to about 13 Å. In some embodiments, the resolution is from about 8 Å to about 14 Å. In some embodiments, the resolution is from about 9 Å to about 15 Å. In some embodiments, the resolution is from about 10 Å to about 16 Å. In some embodiments, the resolution is from about 11 Å to about 17 Å. In some embodiments, the resolution is from about 12 Å to about 18 Å. In some embodiments, the resolution is from about 13 Å to about 19 Å. In some embodiments, the resolution is from about 12 Å to about 20 Å. In some embodiments, the resolution is about 4 Å. In some embodiments, the resolution is about 5 Å. In some embodiments, the resolution is about 6 Å. In some embodiments, the resolution is about 7 Å. In some embodiments, the resolution is about 8 Å. In some embodiments, the resolution is about 9 Å. In some embodiments, the resolution is about 10 Å. In some embodiments, the resolution is about 11 Å. In some embodiments, the resolution is about 12 Å. In some embodiments, the resolution is about 13 Å. In some embodiments, the resolution is about 14 Å. In some embodiments, the resolution is about 15 Å. In some embodiments, the resolution is about 16 Å. In some embodiments, the resolution is about 17 Å. In some embodiments, the resolution is about 18 Å. In some embodiments, the resolution is about 19 Å. In some embodiments, the resolution is about 20 Å.

In some embodiments, the AI technique used in the methods of disclosure predicts the protein structure based on the distances between pairs of amino acids. In some embodiments, the AI technique used in the methods of disclosure predicts the protein structure based on the angles between chemical bonds that connect those amino acids. In some embodiments, the AI technique used in the methods of disclosure predicts the protein structure based on both the protein structure based on the distances between pairs of amino acids and the angles between chemical bonds that connect those amino acids. In some embodiments, the AI technique used in the methods of disclosure predicts protein structure based on end-to-end differentiable deep learning to create mappings end-to-end and use an algorithm to laboriously search for a plausible structure that incorporates those features. In some embodiments, the AI technique used in the methods of disclosure predicts protein structure based on the algorithm disclosed herein as initially published in in AlQuraishi, Cell Systems, 2019, 8: 292-301, incorporated by reference herein.

In some embodiments, the deep neural network used in the methods of the disclosure is a neural network trained for predicting a distance between every pair of amino acid residues in a folded protein. In some embodiments, the deep neural network is a neural network trained for predicting an angle of the joints between consecutive amino acids in a folded protein. In some embodiments, the deep neural network is an end-to-end differentiable deep learning network.

Referring to FIG. 38, which shows a representative flowchart illustrating the architecture of one of the AI techniques suitable for practicing the methods of the disclosure, the Alphafold system, for predicting structure from protein sequence. As a first step, multiple sequences are aligned and the alignments are used together with available databases to train neural networks. In this illustration, the neural network training are focused on two aspects: predicting a distance between every pair of amino acid residues in a folded protein (distance prediction) and predicting an angle of the joints between consecutive amino acids in a folded protein (angle prediction). These two sets of predictions are then used to calculate a score using gradient descent, which is then used to predict the protein 3-D structure.

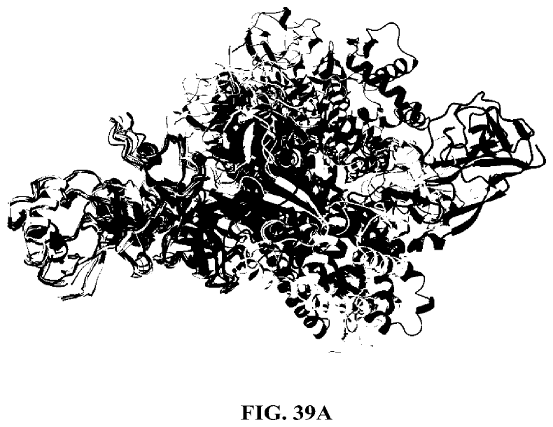

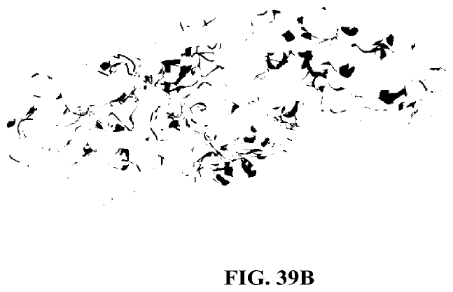

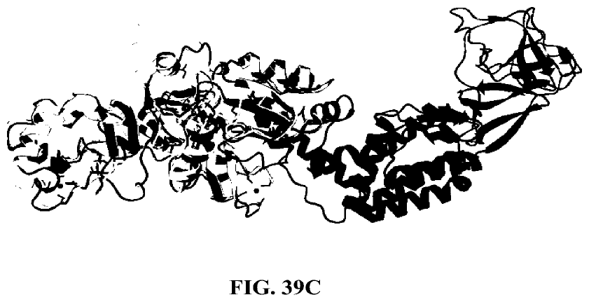

To demonstrate the methods of the disclosure for determining the global protein structure, the Nsp2 protein of SARS CoV2 was used as the protein of interest. The Nsp2 protein of SARS CoV2 has no known function and experiment in SARS CoV1 showed that Nsp2 is not essential but its selection causes a replication defect. A number of high confidence host interactions for Nsp2 were identified using the MS technique. A 3.2 Å SARS CoV2 cryoEM structure was then constructed completely de novo. The experimental model thus built finds no homologous structures in the protein database. It was noted that a 10-amino acid loop and the C-terminus of 120 amino acids in length were missing from this built experimental model (FIG. 39B). The presence of this missing C-terminus was confirmed in a 3.8 Å reconstruction under different conditions (data not shown). However, as it was predicted to be all beta sheets, a de novo structure cannot be built experimentally.

The structure of Nsp2 of SARS CoV2 was also predicted using the AI technique, particularly the AlphaFold program. As shown in FIG. 39A however, the AI prediction by itself fails to recapitulate the correct global protein structure. It appears that the AI technique, such as the AlphaFold program, can have high accuracy in local prediction but lack accuracy in global prediction. In contrast, the protein structure determined by the structural-biology techniques, such as cryoEM, has high accuracy in global prediction, but sometimes lacks accuracy in local prediction as shown in FIG. 39B. By combining the two methodologies as in the methods of the disclosure, a high resolution structure for complete protein can be constructed as shown in FIG. 39C.

In some embodiments, the AI predicted protein structure is divided into overlapping regions of from about 100 to about 300 amino acids in length. In some embodiments, the AI predicted protein structure is divided into overlapping regions of from about 110 to about 280 amino acids in length. In some embodiments, the AI predicted protein structure is divided into overlapping regions of from about 120 to about 260 amino acids in length. In some embodiments, the AI predicted protein structure is divided into overlapping regions of from about 130 to about 240 amino acids in length. In some embodiments, the AI predicted protein structure is divided into overlapping regions of from about 140 to about 220 amino acids in length. In some embodiments, the AI predicted protein structure is divided into overlapping regions of from about 150 to about 200 amino acids in length. In some embodiments, the AI predicted protein structure is divided into overlapping regions of from about 160 to about 180 amino acids in length. In some embodiments, the AI predicted protein structure is divided into overlapping regions of about 100 amino acids in length. In some embodiments, the AI predicted protein structure is divided into overlapping regions of about 110 amino acids in length. In some embodiments, the AI predicted protein structure is divided into overlapping regions of about 120 amino acids in length. In some embodiments, the AI predicted protein structure is divided into overlapping regions of about 130 amino acids in length. In some embodiments, the AI predicted protein structure is divided into overlapping regions of about 140 amino acids in length. In some embodiments, the AI predicted protein structure is divided into overlapping regions of about 150 amino acids in length. In some embodiments, the AI predicted protein structure is divided into overlapping regions of about 160 amino acids in length. In some embodiments, the AI predicted protein structure is divided into overlapping regions of about 170 amino acids in length. In some embodiments, the AI predicted protein structure is divided into overlapping regions of about 180 amino acids in length. In some embodiments, the AI predicted protein structure is divided into overlapping regions of about 190 amino acids in length. In some embodiments, the AI predicted protein structure is divided into overlapping regions of about 200 amino acids in length. In some embodiments, the AI predicted protein structure is divided into overlapping regions of about 210 amino acids in length. In some embodiments, the AI predicted protein structure is divided into overlapping regions of about 220 amino acids in length. In some embodiments, the AI predicted protein structure is divided into overlapping regions of about 230 amino acids in length. In some embodiments, the AI predicted protein structure is divided into overlapping regions of about 240 amino acids in length. In some embodiments, the AI predicted protein structure is divided into overlapping regions of about 250 amino acids in length. In some embodiments, the AI predicted protein structure is divided into overlapping regions of about 260 amino acids in length. In some embodiments, the AI predicted protein structure is divided into overlapping regions of about 270 amino acids in length. In some embodiments, the AI predicted protein structure is divided into overlapping regions of about 280 amino acids in length. In some embodiments, the AI predicted protein structure is divided into overlapping regions of about 290 amino acids in length. In some embodiments, the AI predicted protein structure is divided into overlapping regions of about 300 amino acids in length.

Depending on the length of the regions the AI predicted protein structure is divided into, the length of the overlapping regions may vary. In some embodiments, the regions of the AI predicted protein structure overlap one another by about 10% of the length of the regions. In some embodiments, the regions of the AI predicted protein structure overlap one another by about 15% of the length of the regions. In some embodiments, the regions of the AI predicted protein structure overlap one another by about 20% of the length of the regions. In some embodiments, the regions of the AI predicted protein structure overlap one another by about 25% of the length of the regions. In some embodiments, the regions of the AI predicted protein structure overlap one another by about 30% of the length of the regions. In some embodiments, the regions of the AI predicted protein structure overlap one another by about 35% of the length of the regions. In some embodiments, the regions of the AI predicted protein structure overlap one another by about 40% of the length of the regions. In some embodiments, the regions of the AI predicted protein structure overlap one another by about 45% of the length of the regions. In some embodiments, the regions of the AI predicted protein structure overlap one another by about 50% of the length of the regions.

In some embodiments, the regions of the AI predicted protein structure overlap one another by about 10 amino acid residues. In some embodiments, the regions of the AI predicted protein structure overlap one another by about 15 amino acid residues. In some embodiments, the regions of the AI predicted protein structure overlap one another by about amino acid residues. In some embodiments, the regions of the AI predicted protein structure overlap one another by about 25 amino acid residues. In some embodiments, the regions of the AI predicted protein structure overlap one another by about 30 amino acid residues. In some embodiments, the regions of the AI predicted protein structure overlap one another by about 35 amino acid residues. In some embodiments, the regions of the AI predicted protein structure overlap one another by about 40 amino acid residues. In some embodiments, the regions of the AI predicted protein structure overlap one another by about amino acid residues. In some embodiments, the regions of the AI predicted protein structure overlap one another by about 50 amino acid residues. In some embodiments, the regions of the AI predicted protein structure overlap one another by about 55 amino acid residues. In some embodiments, the regions of the AI predicted protein structure overlap one another by about 60 amino acid residues. In some embodiments, the regions of the AI predicted protein structure overlap one another by about 65 amino acid residues. In some embodiments, the regions of the AI predicted protein structure overlap one another by about amino acid residues. In some embodiments, the regions of the AI predicted protein structure overlap one another by about 75 amino acid residues. In some embodiments, the regions of the AI predicted protein structure overlap one another by about 80 amino acid residues. In some embodiments, the regions of the AI predicted protein structure overlap one another by about 90 amino acid residues. In some embodiments, the regions of the AI predicted protein structure overlap one another by about 100 amino acid residues.

In some embodiments, the AI predicted protein structure is divided into regions of about 100 amino acid residues and overlap one another by about 25 amino acid residues. In some embodiments, the AI predicted protein structure is divided into regions of about 110 amino acid residues and overlap one another by about 30 amino acid residues. In some embodiments, the AI predicted protein structure is divided into regions of about 120 amino acid residues and overlap one another by about 35 amino acid residues. In some embodiments, the AI predicted protein structure is divided into regions of about 130 amino acid residues and overlap one another by about 40 amino acid residues. In some embodiments, the AI predicted protein structure is divided into regions of about 140 amino acid residues and overlap one another by about 45 amino acid residues. In some embodiments, the AI predicted protein structure is divided into regions of about 150 amino acid residues and overlap one another by about 50 amino acid residues. In some embodiments, the AI predicted protein structure is divided into regions of about 160 amino acid residues and overlap one another by about 55 amino acid residues. In some embodiments, the AI predicted protein structure is divided into regions of about 170 amino acid residues and overlap one another by about 60 amino acid residues. In some embodiments, the AI predicted protein structure is divided into regions of about 180 amino acid residues and overlap one another by about 65 amino acid residues. In some embodiments, the AI predicted protein structure is divided into regions of about 190 amino acid residues and overlap one another by about 70 amino acid residues. In some embodiments, the AI predicted protein structure is divided into regions of about 200 amino acid residues and overlap one another by about 75 amino acid residues.

The overlapping regions of the AI predicted protein structure are then globally aligned with the molecular volume of the protein of interest obtained from the structural-biology technique using one or a plurality of global rigid-body fitting packages to obtain a global rigid-body transformation. Publically available global rigid-body fitting packages includes, but not limited to, Situs (available at situs.biomachina.org) and Chimera (available at www.cgl.ucsf.edu/chimera). In some embodiments, the global rigid-body fitting is performed using the Situs package. In some embodiments, the global rigid-body fitting is performed using the Chimera package.

The overlapping regions of the AI predicted protein structure with top scoring fits are selected and further examined to generate new region boundaries. If necessary, another run of global rigid-body fitting can be performed using the selected top-scoring regions. The finally selected top-scoring regions are combined into a complete protein structure, which is then refined into the molecular volume of the protein of interest obtained from the structural-biology technique. This refinement of the protein structure can be performed using publically available algorithms, such as Rosetta Relax (see rosettacommons.org).

### EXEMPLIFICATION

Representative examples of the disclosed methods and systems are illustrated in the following non-limiting methods and examples.

### Materials and Methods

Cells

HEK293T/17 (HEK293T) cells were procured from the UCSF Cell Culture Facility, and are available through UCSF's Cell and Genome Engineering Core (https://cgec.ucsf.edu/cell-culture-and-banking-services). HEK293T cells were cultured in Dulbecco's Modified Eagle's Medium (DMEM) (Corning) supplemented with 10% Fetal Bovine Serum (FBS) (Gibco, Life Technologies) and 1% Penicillin-Streptomycin (Corning) and maintained at 37° C. in a humidified atmosphere of 5% CO₂. STR analysis by the Berkeley Cell Culture Facility on Aug. 8, 2017 authenticates these as HEK293T cells with 94% probability.

HeLaM cells (RRID: CVCL_R965) were originally obtained from the laboratory of M. S. Robinson (CIMR, University of Cambridge, UK) and routinely tested for mycoplasma contamination. HeLaM cells were grown in DMEM supplemented with 10% FBS, 100 U/ml penicillin, 100 μg/ml streptomycin and 2 mM glutamine at 37° C. in a 5% CO₂ humidified incubator.

A549 cells stably expressing ACE2 (A549-ACE2) were a kind gift from Dr. Olivier Schwartz. A549-ACE2 cells were cultured in DMEM supplemented with 10% FBS, blasticidin (20 μg/ml, Sigma) and maintained at 37° C. with 5% CO₂. STR analysis by the Berkeley Cell Culture Facility on Jul. 17, 2020 authenticates these as A549 cells with 100% probability.

Caco-2 cells were cultured in DMEM with GlutaMAX and pyruvate (Gibco, 10569010) and supplemented with 20% FBS (Gibco, 26140079). For Caco-2 cells utilized in Cas9-RNP knockouts, STR analysis by the Berkeley Cell Culture Facility on Apr. 23, 2020 authenticates these as Caco-2 cells with 100% probability.

Vero E6 cells were purchased from ATCC and thus authenticated (VERO C1008 [Vero 76, clone E6, Vero E6] (ATCC, CRL-1586). Vero E6 cells tested negative for mycoplasma contamination. Vero E6 cells were cultured in DMEM (Corning) supplemented with 10% Fetal Bovine Serum (FBS) (Gibco, Life Technologies) and 1% Penicillin-Streptomycin (Corning) and maintained at 37° C. in a humidified atmosphere of 5% CO₂.

Coronavirus Annotation and Plasmid Cloning

SARS-CoV-1 isolate Tor2 (NC_004718) and MERS-CoV (NC_019843) were downloaded from Genbank and utilized to design 2×-Strep tagged expression constructs of open reading frames (Orfs) and proteolytically mature nonstructural proteins (Nsps) derived from Orf1ab (with N-terminal methionines and stop codons added as necessary). Protein termini were analyzed for predicted acylation motifs, signal peptides, and transmembrane regions, and either the N- or C-terminus was chosen for tagging as appropriate. Finally, reading frames were codon optimized and cloned into pLVX-EF1alpha-IRES-Puro (Takara/Clontech) including a 5′ Kozak motif.

Immunofluorescence Microscopy of Viral Protein Constructs

Approximately 60,000 HeLaM cells were seeded onto glass coverslips in a 12-well dish and grown overnight. The cells were transfected using 0.5 μg of plasmid DNA and either polyethylenimine (Polysciences) or Fugene HD (Promega; 1 part DNA to 3 parts transfection reagent) and grown for a further 16 hours.

Transfected cells were fixed with 4% paraformaldehyde (Polysciences) in PBS at room temperature for 15 minutes. The fixative was removed and quenched using 0.1 M glycine in PBS. The cells were permeabilized using 0.1% saponin in PBS containing 10% FBS. The cells were stained with the indicated primary and secondary antibodies for 1 hour at room temperature. The coverslips were mounted onto microscope slides using ProLong Gold antifade reagent (ThermoFisher) and imaged using a UplanApo 60×oil (NA 1.4) immersion objective on a Olympus BX61 motorized wide-field epifluorescence microscope. Images were captured using a Hamamatsu Orca monochrome camera and processed using ImageJ.

To gain insight into the intracellular distribution of each Strep-tagged construct, approximately 100 cells per transfection were manually scored. Each construct was assigned an intracellular distribution in relation to the plasma membrane, endoplasmic reticulum, Golgi, cytoplasm and mitochondria (scored out of 7). In several instances the viral proteins were observed on membranes which did not fit any of the basic categories so were defined as being localized on undefined membranes. Many of the constructs had several localizations so this was also reflected in the scoring. The scoring also took into account the impact of expression level on the localization of the constructs.

Meta Analysis of Immunofluorescence Data

The data concerning viral protein location was first sorted for all Strep-tagged viral proteins expressed individually in three heatmaps (one per virus) using a custom R script (“pheatmap” package). The information concerning protein localization during SARS-CoV-2 infection was added as a square border color code in the first heatmap, to compare the two different localization patterns. In order to compare the predicted versus the experimentally determined locations, the top scoring sequence-based localization prediction for each protein was taken from DeepLoc (J. J. Almagro Armenteros, et al. DeepLoc: prediction of protein subcellular localization using deep learning. Bioinformatics. 33, 3387-3395 (2017)) if the score was bigger than 1. When more than one localization can be assigned to the same protein, as many top scoring ones were taken as the number of experimentally assigned localizations available for the same protein. Finally, for each cell compartment, the number of experimentally assigned viral proteins was counted, and the subset of them predicted to that same compartment as “correct predictions.” To compare changes in protein interactions with changes in protein localization (Strep-tagged experiment versus sequence-based prediction), the Jaccard index of prey overlap was calculated for each viral protein (SARS-CoV-2 vs. SARS-CoV-1 and SARS-CoV-2 vs. MERS-CoV) and plotted together, for proteins with the same localization and for proteins with different localization.

Generation of Polyclonal Sheep Antibodies Targeting SARS-CoV-2 Proteins

Sheep were immunized with individual N-terminal GST-tagged SARS-CoV-2 recombinant proteins or N-terminal MBP-tagged proteins (for SARS-CoV-2 S, S-RBD, and Orf7a), followed by up to 5 booster injections four weeks apart from each other. Sheep were subsequently bled and IgGs were affinity purified using the specific recombinant N-terminal maltose binding protein (MBP)-tagged viral proteins. Each antiserum specifically recognized the appropriate native viral protein. Characterisation of each antiserum by western blotting, immunoprecipitation and immunofluorescence of virus-infected and mock-infected cells were described elsewhere. All antibodies generated can be requested at https://mrcppu-covid.bio/. Also see Table 1.

TABLE 1

| | Antigen | Working | | Catalogue |
|---|---|---|---|---|
| Reagent | Species | Dilution | Supplier | Number |
| Sheep anti- | SARS-COV-2 | 1/200 | MRC PPU Reagents | DA103 |
| Nsp1 | | | and Services | |
| Sheep anti- | SARS-COV-2 | 1/200 | MRC PPU Reagents | DA105 |
| Nsp2 | | | and Services | |
| Sheep anti- | SARS-COV-2 | 1/200 | MRC PPU Reagents | DA118 |
| Nsp5 | | | and Services | |
| Sheep anti- | SARS-COV-2 | 1/200 | MRC PPU Reagents | DA093 |
| Nsp7 | | | and Services | |
| Sheep anti- | SARS-COV-2 | 1/200 | MRC PPU Reagents | DA110 |
| Nsp8 | | | and Services | |
| Sheep anti- | SARS-COV-2 | 1/200 | MRC PPU Reagents | DA094 |
| Nsp9 | | | and Services | |
| Sheep anti- | SARS-COV-2 | 1/200 | MRC PPU Reagents | DA091 |
| Nsp10 | | | and Services | |
| Sheep anti- | SARS-COV-2 | 1/200 | MRC PPU Reagents | DA111 |
| Nsp13 | | | and Services | |
| Sheep anti- | SARS-COV-2 | 1/200 | MRC PPU Reagents | DA112 |
| Nsp14 | | | and Services | |
| Sheep anti-M | SARS-COV-2 | 1/200 | MRC PPU Reagents | DA107 |
| Protein | | | and Services | |
| Sheep anti- | SARS-COV-2 | 1/200 | MRC PPU Reagents | DA102 |
| Orf3a | | | and Services | |
| Sheep anti- | SARS-COV-2 | 1/200 | MRC PPU Reagents | DA087 |
| Orf6 | | | and Services | |
| Sheep anti- | SARS-COV-2 | 1/200 | MRC PPU Reagents | DA092 |
| Orf7b | | | and Services | |
| Sheep anti- | SARS-COV-2 | 1/200 | MRC PPU Reagents | DA088 |
| Orf8 | | | and Services | |
| Sheep anti- | SARS-COV-2 | 1/200 | MRC PPU Reagents | DA089 |
| Orf9a (Orf9b | | | and Services | |
| in this | | | | |
| manuscript) | | | | |
| Mouse anti- | N/A | 1/5000 | Qiagen | 34850 |
| Strep | | | | |
| Mouse anti- | N/A | 1/1000 | IBA Lifesciences | 2-1507- |
| StrepMAB | | | | 001 |
| Rabbit anti- | Human | 1/500 | Synaptic Systems | 110 053 |
| STX5 | | | | |
| Rabbit anti- | Human | | Cell Signaling | 3177S |
| BiP | | | | |
| Rabbit anti- | Human | | Cell Signaling | 3501S |
| PDI | | | | |
| Mouse anti- | Human | 1/200 | Alexis Biologicals | G1/93 |
| ERGIC-53 | | | | |
| Rabbit anti- | Human | 1/1000 | Proteintech | 11802-1- |
| TOM20 | | | | AP |
| Mouse anti- | Human | 1/500 | Santa Cruz | sc-390545 |
| TOM70 | | | | |
| Mouse anti- | Human | 1/200 | BD | 610457 |
| EEA1 | | | | |
| Goat anti- | Rabbit | 1/500 | ThermoFisher | A32731 |
| Rabbit Alexa | | | Scientific | |
| Fluor Plus 488 | | | | |
| Goat anti- | Mouse | 1/1000 | ThermoFisher | A32742 |
| Mouse Alexa | | | Scientific | |
| Fluor Plus 594 | | | | |
| Goat anti- | Mouse | 1/20,000 | BioRad | 1706516 |
| Mouse HRP | | | | |
| AF568-labeled | Sheep | 1/400 | Invitrogen | A21099 |
| donkey-anti- | | | | |
| sheep | | | | |
| AF647-labeled | | 1/400 | Hypermol | 8817-01 |
| Phalloidin | | | | |
| AF488-labeled | Rabbit | 1/400 | Invitrogen | A21441 |
| chicken-anti- | | | | |
| rabbit | | | | |
| AF488-labeled | Mouse | 1/400 | Invitrogen | A21200 |
| chicken-anti- | | | | |
| mouse | | | | |
| Rabbit anti-NP | SARS-COV-2 | 1/10,000 | Garcis-Sastre Lab | |
| antisera | | | | |

Immunofluorescence Microscopy of Infected Caco-2 Cells

For infection experiments in human colon epithelial Caco-2 cells (ATCC, HTB-37), SARS-CoV-2 isolate Muc-IMB-1, kindly provided by the Bundeswehr Institute of Microbiology, Munich, Germany, was used. SARS-CoV-2 was propagated in Vero E6 cells in DMEM supplemented with 2% FBS. All work involving live SARS-CoV-2 was performed in the BSL3 facility of the Institute of Virology, University Hospital Freiburg, and was approved according to the German Act of Genetic Engineering by the local authority (Regierungspraesidium Tuebingen, permit UNI.FRK.05.16/05).

Caco-2 human colon epithelial cells seeded on glass coverslips were infected with SARS-CoV-2 (Strain Muc-IMB-1/2020, second passage on Vero E6 cells (2×10⁶ PFU/ml)) at an MOI of 0.1. At 24 hours post-infection, cells were washed with PBS and fixed in 4% paraformaldehyde in PBS for 20 minutes at room temperature, followed by 5 minutes of quenching in 0.1 M glycine in PBS at room temperature. Cells were permeabilized and blocked in 0.1% saponin in PBS supplemented with 10% fetal calf serum for 45 minutes at room temperature and incubated with primary antibodies for 1 hour at room temperature. After washing 15 minutes with blocking solution, AF568-labeled donkey-anti-sheep (Invitrogen, #A21099; 1:400) secondary antibody as well as AF4647-labeled Phalloidin (Hypermol, #8817-01, 1:400) were applied for 1 hour at room temperature. Subsequent washing was followed by embedding in Diamond Antifade Mountant with DAPI. Fluorescence images were generated using a LSM800 confocal laser-scanning microscope (Zeiss) equipped with a 63×, 1.4 NA oil objective and Airyscan detector and the Zen blue software (Zeiss) and processed with Zen blue software and ImageJ/Fiji.

Transfection and Cell Harvest for Immunoprecipitation Experiments

For each affinity purification (SARS-CoV-1 baits, MERS-CoV baits, GFP-2×Strep, or empty vector controls), ten million HEK293T cells were transfected with up to 15 μg of individual expression constructs using PolyJet transfection reagent (SignaGen Laboratories) at a 1:3 μg:μl ratio of plasmid to transfection reagent based on manufacturer's protocol. After more than 38 hours, cells were dissociated at room temperature using 10 ml PBS without calcium and magnesium (D-PBS) with 10 mM EDTA for at least 5 minutes, pelleted by centrifugation at 200×g, at 4° C. for 5 minutes, washed with 10 ml D-PBS, pelleted once more and frozen on dry ice before storage at −80° C. for later immunoprecipitation analysis. For each bait, three independent biological replicates were prepared.

Anti-Strep-Tag Affinity Purification

Frozen cell pellets were thawed on ice for 15-20 minutes and suspended in 1 ml Lysis Buffer [IP Buffer (50 mM Tris-HCl, pH 7.4 at 4° C., 150 mM NaCl, 1 mM EDTA) supplemented with 0.5% Nonidet P 40 Substitute (NP-40; Fluka Analytical) and cOmplete mini EDTA-free protease and PhosSTOP phosphatase inhibitor cocktails (Roche)]. Samples were then freeze-fractured by refreezing on dry ice for 10-20 minutes, then rethawed and incubated on a tube rotator for 30 minutes at 4° C. Debris was pelleted by centrifugation at 13,000×g, at 4° C. for 15 minutes. Up to 56 samples were arrayed into a 96-well Deepwell plate for affinity purification on the KingFisher Flex Purification System (Thermo Scientific) as follows: MagStrep “type3” beads (30 μl; IBA Lifesciences) were equilibrated twice with 1 ml Wash Buffer (IP Buffer supplemented with 0.05% NP-40) and incubated with 0.95 ml lysate for 2 hours. Beads were washed three times with 1 ml Wash Buffer and then once with 1 ml IP Buffer. Beads were released into 75 μl Denaturation-Reduction Buffer (2 M urea, 50 mM Tris-HCl pH 8.0, 1 mM DTT) in advance of on-bead digestion. All automated protocol steps were performed at 4° C. using the slow mix speed and the following mix times: 30 seconds for equilibration/wash steps, 2 hours for binding, and 1 minute for final bead release. Three 10 second bead collection times were used between all steps.

On-Bead Digestion for Affinity Purification

Bead-bound proteins were denatured and reduced at 37° C. for 30 minutes, alkylated in the dark with 3 mM iodoacetamide for 45 minutes at room temperature, and quenched with 3 mM DTT for 10 minutes. To offset evaporation, 22.5 μl 50 mM Tris-HCl, pH 8.0 were added prior to trypsin digestion. Proteins were then incubated at 37° C., initially for 4 hours with 1.5 μl trypsin (0.5 μg/μl; Promega) and then another 1-2 hours with 0.5 μl additional trypsin. All steps were performed with constant shaking at 1,100 rpm on a ThermoMixer C incubator. Resulting peptides were combined with 50 μl 50 mM Tris-HCl, pH 8.0 used to rinse beads and acidified with trifluoroacetic acid (0.5% final, pH<2.0). Acidified peptides were desalted for MS analysis using a BioPureSPE Mini 96-Well Plate (20 mg PROTO 300 C18; The Nest Group, Inc.) according to standard protocols.

Mass Spectrometry Operation and Peptide Search

Samples were re-suspended in 4% formic acid, 2% acetonitrile solution, and separated by a reversed-phase gradient over a nanoflow C18 column (Dr. Maisch). Each sample was directly injected via a Easy-nLC 1200 (Thermo Fisher Scientific) into a Q-Exactive Plus mass spectrometer (Thermo Fisher Scientific) and analyzed with a 75 minute acquisition, with all MS1 and MS2 spectra collected in the orbitrap; data were acquired using the Thermo software Xcalibur (4.2.47) and Tune (2.11 QF1 Build 3006). For all acquisitions, QCloud was used to control instrument longitudinal performance during the project (C. Chiva, et al., QCloud: A cloud-based quality control system for mass spectrometry-based proteomics laboratories. PLoS One. 13, e0189209 (2018)). All proteomic data was searched against the human proteome (uniprot reviewed sequences downloaded Feb. 28, 2020), EGFP sequence, and the SARS-CoV or MERS protein sequences using the default settings for MaxQuant (version 1.6.12.0) (J. Cox, M. Mann, MaxQuant enables high peptide identification rates, individualized p.p.b.-range mass accuracies and proteome-wide protein quantification. Nat. Biotechnol. 26, 1367-1372 (2008)). Detected peptides and proteins were filtered to 1% false discovery rate in MaxQuant. All MS raw data and search results files have been deposited to the ProteomeXchange Consortium via the PRIDE partner repository with the dataset (identifier PXD PXDO21588, Username: reviewer_pxd021588@ebi.ac.uk, password: B5Ho3HES).

High-Confidence Protein Interaction Scoring

Identified proteins were then subjected to protein-protein interaction scoring with both SAINTexpress (version 3.6.3) and MiST (https://github.com/kroganlab/mist) (Teo, et al. SAINTexpress: improvements and additional features in Significance Analysis of INTeractome software. J. Proteomics. 100, 37-43 (2014); S. Jäger, et al., Global landscape of HIV-human protein complexes. Nature. 481, 365-370 (2011)). A two-step filtering strategy was applied to determine the final list of reported interactors, which relied on two different scoring stringency cut-offs. In the first step, all protein interactions that had a MiST score≥a SAINTexpress Bayesian false-discovery rate (BFDR)≤0.05, and an average spectral count≥2 were chosen. For all proteins that fulfilled these criteria, information about the stable protein complexes that they participated in was extracted from the CORUM (M. Giurgiu, et al., CORUM: the comprehensive resource of mammalian protein complexes-2019. Nucleic Acids Res. 47, D559-D563 (2019)) database of known protein complexes. In the second step, the stringency was relaxed, and additional interactors that (1) formed complexes with interactors determined in filtering step 1 and (2) fulfilled the following criteria: MiST score≥0.6, SAINTexpress BFDR≤0.05, and average spectral counts≥2, were recovered. Proteins that fulfilled filtering criteria in either step 1 or step 2 were considered to be high-confidence protein-protein interactions (HC-PPIs).

Using this filtering criteria, nearly all of the baits recovered a number of HC-PPIs in close alignment with previous datasets reporting an average of around 6 PPIs per bait (E. L. Huttlin, et al., The BioPlex Network: A Systematic Exploration of the Human Interactome. Cell. 162, 425-440 (2015)). However, for a subset of baits, a much higher number of PPIs that passed these filtering criteria were observed. For these baits, the MiST scoring was instead performed using a larger in-house database of 87 baits that were prepared and processed in an analogous manner to this SARS-CoV-2 dataset. This was done to provide a more comprehensive collection of baits for comparison, to minimize the classification of non-specifically binding background proteins as HC-PPIs. This was performed for SARS-CoV-1 baits (M, Nsp12, Nsp13, Nsp8, and Orf7b), MERS-CoV baits (Nsp13, Nsp2, and Orf4a), and SARS-CoV-2 Nsp16. SARS-CoV-2 Nsp16 MiST was scored using the in-house database as well as all previous SARS-CoV-2 data (Gordon, et al. A SARS-CoV-2 protein interaction map reveals targets for drug repurposing. Nature (2020)).

Hierarchical Clustering of Virus-Human Protein Interactions

Hierarchical clustering was performed on interactions for (1) viral bait proteins shared across all three viruses (LIST) and (2) passed the high-confidence scoring criteria (MiST score≥0.6, SAINTexpress BFDR≤0.05, and average spectral counts≥2) in at least one virus. Clustering was performed using a new Interaction Score (K), which was defined as the average between the MiST and Saint score for each virus-human interaction. This was done to provide a single score that captured the benefits from each scoring method. Clustering was performed using the ComplexHeatmap package in R, using the “average” clustering method and “euclidean” distance metric. K-means clustering (k=7) was applied to capture all possible combinations of interaction patterns between viruses.

Gene Ontology Enrichment Analysis on Clusters

Sets of genes found in 7 clusters were tested for enrichment of Gene Ontology (GO) terms, which was performed using the enricher function of clusterProfiler package in R (G. Yu, et al., clusterProfiler: an R package for comparing biological themes among gene clusters. OMICS. 16, 284-287 (2012)). The GO terms were obtained from the C5 collection of Molecular Signature Database (MSigDBv7.1) and include Biological Process, Cellular Component, and Molecular Function ontologies. Significant GO terms were identified (adjusted p-value<0.05) and further refined to select non-redundant terms. To select non-redundant gene sets, a GO term tree based on distances (1−Jaccard Similarity Coefficients of shared genes) between the significant terms was first constructed. The GO term tree was cut at a specific level (h=0.99) to identify clusters of non-redundant gene sets. For results with multiple significant terms belonging to the same cluster, the term with the lowest adjusted p-value was selected.

Sequence Similarity Analysis

Protein sequence similarity was assessed by comparing the protein sequences from SARS-CoV-1 and MERS-CoV to SARS-CoV-2 for orthologous viral bait proteins. The corresponding protein-protein interaction similarity was represented by a Jaccard index, using the high-confidence interactomes for each virus.

Gene Ontology Enrichment and PPI Similarity Analysis

The high-confidence interactors of the three viruses were tested for enrichment of GO terms as described above. Next, GO terms that are significantly enriched (adjusted p-value<0.05) in all 3 viruses were selected. For each enriched term, the list of its associated genes was generated, and the Jaccard Index of pairwise comparisons of 3 viruses computed.

Orthologous Versus Non-Orthologous Interactions Analysis

For a given pair of viruses, all pairs of baits that share interactors were identified and categorized into “orthologous” and “non-orthologous” groups based on whether the two baits were orthologs or not. Then, the total number of shared interactors in each group was summed up to calculate the corresponding fractions. This was performed for all pairwise combinations of the three viruses.

Structural Modeling and Comparison of MERS-CoV Orf4a and SARS-CoV-2 Nsp8

To obtain a sensitive sequence comparison between MERS-CoV Orf4a and SARS-CoV-2 Nsp8, their homologs were taken into consideration. First, homologs of these proteins were searched for in the UniRef30 database using hhblits (1 iteration, E-value cutoff 1e-3) (M. Remmert, et al., HHblits: lightning-fast iterative protein sequence searching by HMM-HMM alignment. Nat. Methods. 9, 173-175 (2011)). Subsequently, the resulting alignments were filtered to include only sequences with at least 80% coverage to the corresponding query sequence, and hidden Markov models (HMMs) were created using hhmake. Finally, the HMMs of Orf4a andNsp8 homologs were locally aligned using hhalign. The structure of Orf4a was predicted de novo using trRosetta (J. Yang, et al., Improved protein structure prediction using predicted interresidue orientations. Proc. Natl. Acad. Sci. U.S.A 117, 1496-1503 (2020)). To provide greater coverage than that provided by experimental structures, SARS-CoV-2 Nsp8 was modeled using the structure of its SARS-CoV homolog as template (PDB: 2AHM) (Y. Zhai, et al., Insights into SARS-CoV transcription and replication from the structure of the nsp7-nsp8 hexadecamer. Nat. Struct. Mol. Biol. 12, 980-986 (2005)) using SWISS-MODEL (A. Waterhouse, et al., SWISS-MODEL: homology modelling of protein structures and complexes. Nucleic Acids Res. 46, W296-W303 (2018)). To search for local structural similarities between Orf4a and Nsp8, Geometricus, a structure embedding tool based on 3D rotation invariant moments, was used (J. Durairaj, et al., Geometricus Represents Protein Structures as Shape-mers Derived from Moment Invariants (2020), p. 2020.09.07.285569). This generates so-called shape-mers, analogous to sequence k-mers. The structures were fragmented into overlapping k-mers based on the sequence (k=20) and into overlapping spheres surrounding each residue (radius=15 Å). To ensure that the similarities found between these distinct structures were significant, a high resolution of 7 was used to define the shape-mers. This resulted in the identification of 4 different shape-mers common to Orf4a and Nsp8. The entire Orf4a structure was aligned with residues 96 to 191 of the Nsp8 structure (i.e., after removal of the long N-terminal helix) using the Caretta structural alignment algorithm detailed by (M. Akdel, et al., Caretta—A multiple protein structure alignment and feature extraction suite. Comput. Struct. Biotechnol. J. 18, 981-992 (2020)), using 3D rotation invariant moments (Durairaj et al. 2020) for initial superposition. The parameters were optimized to maximize the Caretta score. The resulting alignment used k=30, radius=16 Å, gap open penalty=0.05, and gap extend penalty=0.005, and had a root-mean-square deviation (RMSD) of 7.6 Å across 66 aligning residues.

Differential Interaction Score (DIS) Analysis

A differential interaction score (DIS) was calculated for interactions that (1) originated from viral bait proteins shared across all three viruses and (2) passed the high-confidence scoring criteria (MiST score≥0.6, SAINTexpress BFDR≤0.05, and average spectral counts≥2) in at least one virus. The DIS was defined to be the difference between the interaction scores (K) from each virus. DIS near 0 indicates that the interaction is confidently shared between the two viruses being compared, while a DIS near −1 or +1 indicates that the host protein interaction is specific for one virus or the other. A fourth DIS (SARS-MERS) was computed by averaging K from SARS-CoV-1 and SARS-CoV-2 prior to calculating the difference with MERS-CoV. Here, a DIS near +1 indicates SARS-specific interactions (shared between SARS-CoV-1 and SARS-CoV-2 but absent in MERS-CoV), a DIS near −1 indicates MERS-specific interactions (present in MERS-CoV and absent or lowly confident in both SARS-CoVs), and a DIS near 0 indicates interactions shared between all three viruses.

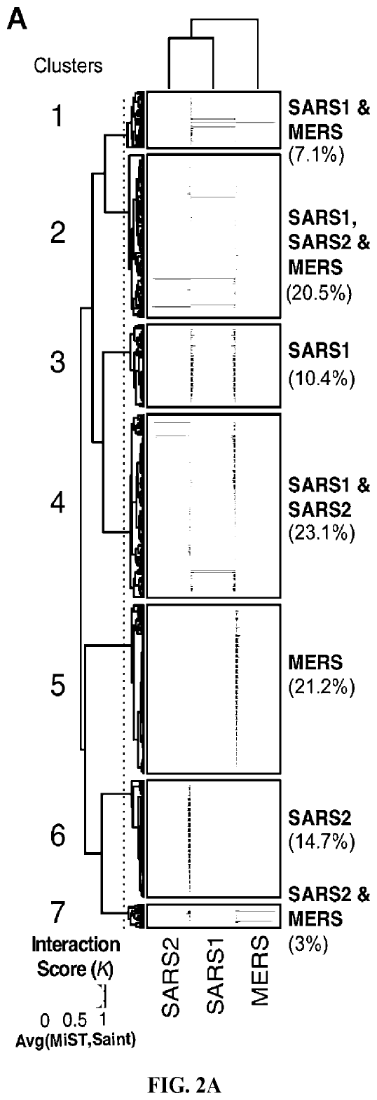

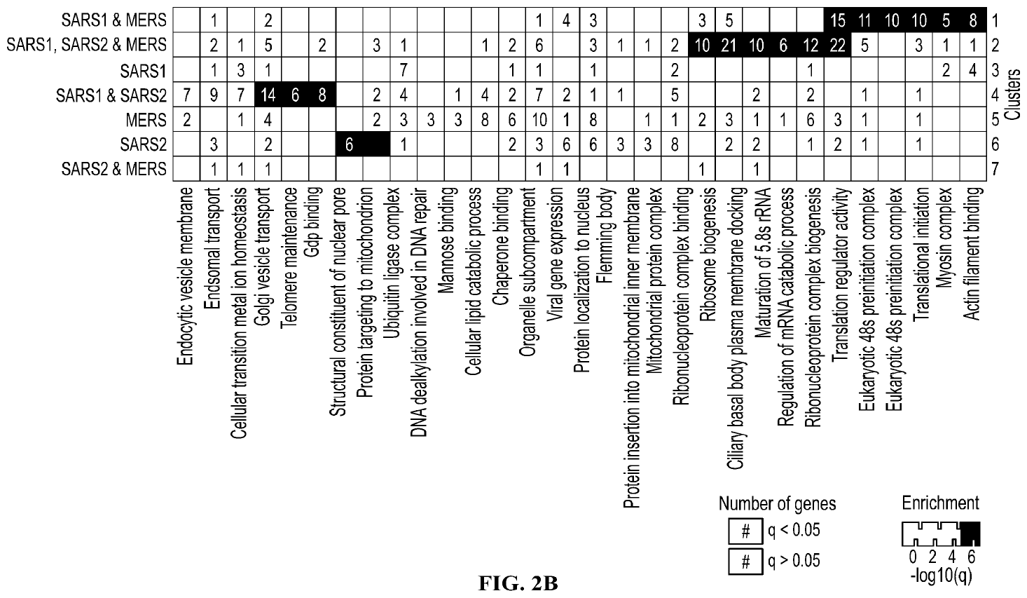

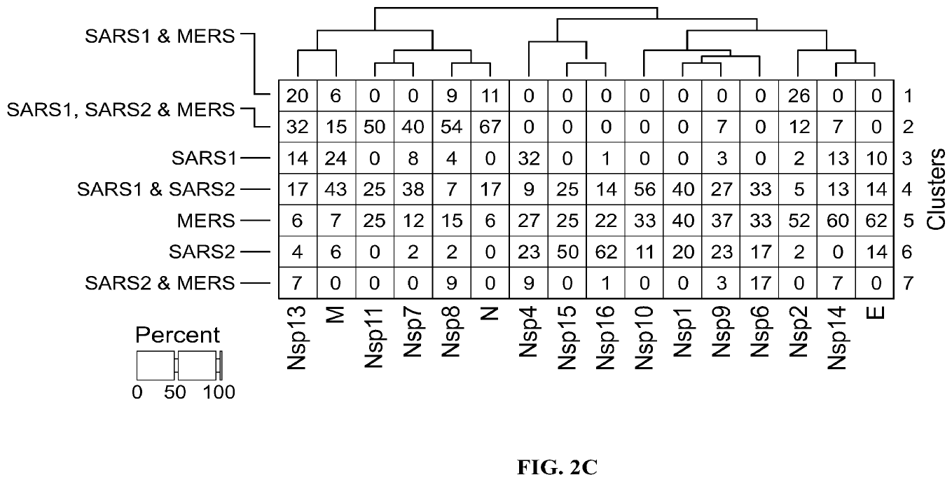

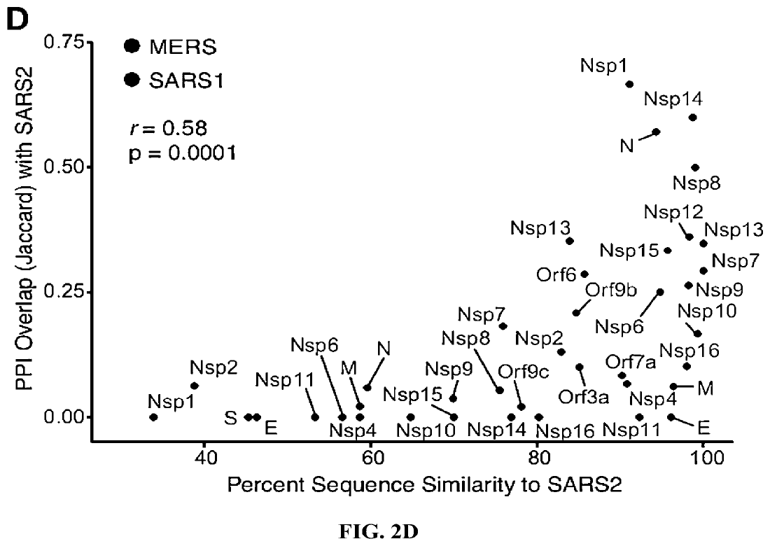

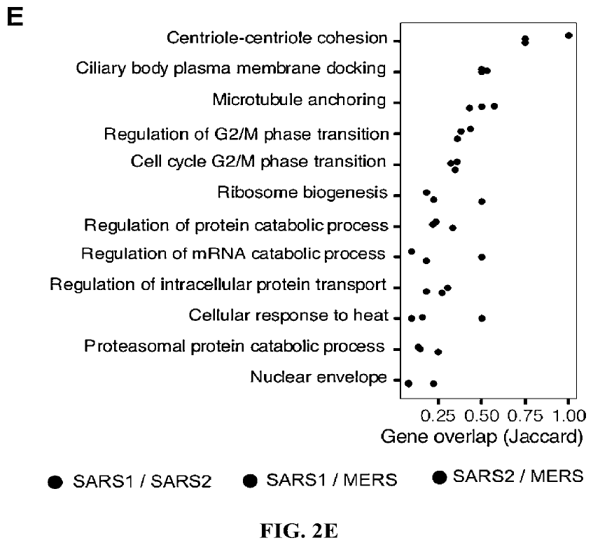

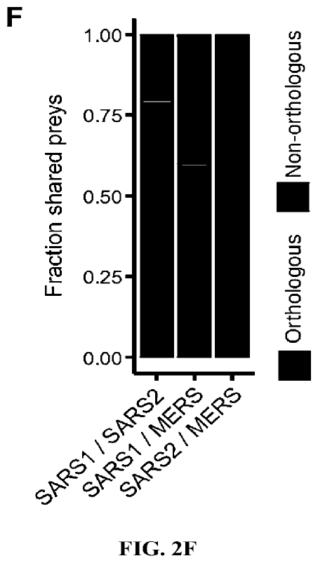

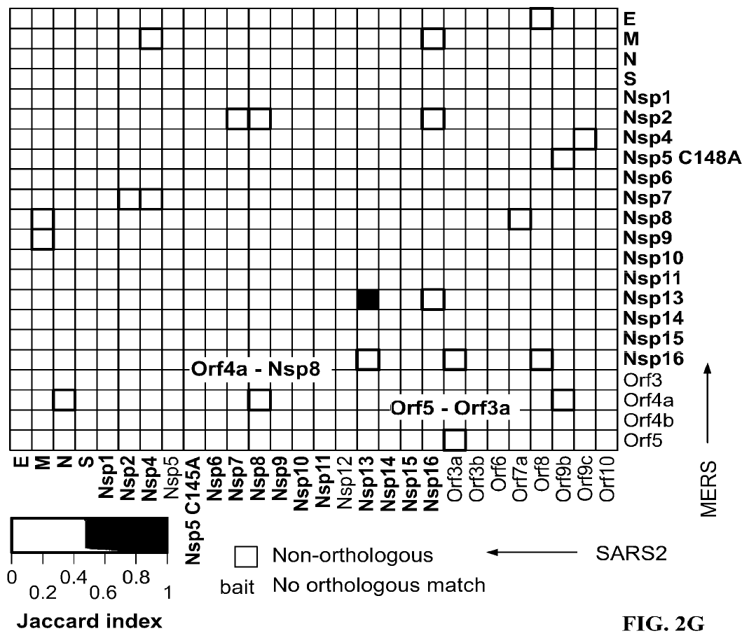

For each pairwise virus comparison, as well as the SARS-MERS comparison, DIS was defined based on cluster membership of interactions (FIG. 2A). For the SARS2-SARS1 comparison, interactions from every cluster except 5 were used, as those interactions are considered absent from both SARS-CoV-2 and SARS-CoV-1. For the SARS2-MERS comparison, interactions from all clusters except 3 were used. For the SARS1-MERS comparison, interactions from all clusters except 6 were used. For the SARS-MERS comparison, only interactions from clusters 2, 4, and 5 were used.

Referring to FIG. 2A, clustering analysis (k-means) of interactors from SARS-CoV-2, SARS-CoV-1, and MERS-CoV weighted according to the average between their MIST and Saint scores (interaction score K) and percentages of total interactions is shown. Included are only viral protein baits represented amongst all three viruses and interactions that pass the high-confidence scoring threshold for at least one virus. Seven clusters highlight all possible scenarios of shared versus unique interactions.

Network Generation and Visualization

Protein-protein interaction networks were generated in Cytoscape (P. Shannon, et al., Cytoscape: a software environment for integrated models of biomolecular interaction networks. Genome Res. 13, 2498-2504 (2003)) and subsequently annotated using Adobe Illustrator. Host-host physical interactions, protein complex definitions, and biological process groupings were derived from CORUM (M. Giurgiu, et al., CORUM: the comprehensive resource of mammalian protein complexes-2019. Nucleic Acids Res. 47, D559-D563 (2019)), Gene Ontology (biological process), and manually curated from literature sources. All networks were deposited in NDEx (R. T. Pillich, et al., NDEx: A Community Resource for Sharing and Publishing of Biological Networks. Methods Mol. Biol. 1558, 271-301 (2017)).

siRNA Library and Transfection in A549-ACE2 Cells

An OnTargetPlus siRNA SMARTpool library (Horizon Discovery) was purchased targeting 331 of the 332 human proteins previously identified to bind SARS-CoV-2 (D. E. Gordon, et al., A SARS-CoV-2 protein interaction map reveals targets for drug repurposing. Nature (2020)) (PDE4DIP was not available for purchase and excluded from the assay). This library was arrayed in 96-well format, with each plate also including two non-targeting siRNAs and one siRNA pool targeting ACE2 (see Table 2 Å provided in U.S. Provisional Application No. 63/091,929 filed on Oct. 15, 2020, expressly incorporated by reference herein). The siRNA library was transfected into A549 cells stably expressing ACE2 (A549-ACE2, kindly provided by Dr. Olivier Schwartz), using Lipofectamine RNAiMAX reagent (Thermo Fisher). Briefly, 6 pmoles of each siRNA pool were mixed with 0.25 μl RNAiMAX transfection reagent and OptiMEM (Thermo Fisher) in a total volume of 20 μl. After a 5 minute incubation period, the transfection mix was added to cells seeded in a 96-well format. 24 hours post-transfection, the cells were subjected to SARS-CoV-2 infection as described in “Viral infection and quantification assay in A549-ACE2 cells,” or incubated for 72 hours to assess cell viability using the CellTiter-Glo luminescent viability assay according to the manufacturer's protocol (Promega). Luminescence was measured in a Tecan Infinity 2000 plate reader, and percentage viability calculated relative to untreated cells (100% viability) and cells lysed with 20% ethanol or 4% formalin (0% viability), included in each experiment.

Viral Infection and Quantification Assay in A549-ACE2 Cells

Cells seeded in a 96-well format were inoculated with a SARS-CoV-2 stock (BetaCoV/France/IDF0372/2020 strain, generated and propagated once in Vero E6 cells and a kind gift from the National Reference Centre for Respiratory Viruses at Institut Pasteur, Paris, originally supplied through the European Virus Archive goes Global platform) at a MOI of 0.1 PFU per cell. Following a one hour incubation period at 37° C., the virus inoculum was removed, and replaced by DMEM containing 2% FBS (Gibco, Thermo Fisher). 72 hours post-infection the cell culture supernatant was collected, heat inactivated at 95° C. for 5 minutes and used for RT-qPCR analysis to quantify viral genomes present in the supernatant. Briefly, SARS-CoV-2 specific primers targeting the N gene region: 5′-TAATCAGACAAGGAACTGATTA-3′ (Forward) and 5′-CGAAGGTGTGACTTCCATG-3′ (Reverse) (D. K. W. Chu, et al., Molecular Diagnosis of a Novel Coronavirus (2019-nCoV) Causing an Outbreak of Pneumonia. Clin. Chem. 66, 549-555 (2020)) were used with the Luna® Universal One-Step RT-qPCR Kit (New England Biolabs) in an Applied Biosystems QuantStudio 6 thermocycler, with the following cycling conditions: 55° C. for 10 minutes, 95° C. for 1 minute, and 40 cycles of 95° C. for 10 seconds, followed by 60° C. for 1 minute. The number of viral genomes is expressed as PFU equivalents/ml, and was calculated by performing a standard curve with RNA derived from a viral stock with a known viral titer.

Knockdown Validation with qRT-PCR in A549-ACE2 Cells

Gene-specific quantitative PCR primers targeting all genes represented in the OnTargetPlus library were purchased and arrayed in a 96-well format identical to that of the siRNA library (IDT; see Table 2B provided in U.S. Provisional Application No. 63/091,929 filed on Oct. 15, 2020, expressly incorporated by reference herein). A549-ACE2 cells treated with siRNA were lysed using the Luna® Cell Ready Lysis Module (New England Biolabs) following the manufacturer's protocol. The lysate was used directly for gene quantification by RT-qPCR with the Luna® Universal One-Step RT-qPCR Kit (New England Biolabs), using the gene-specific PCR primers and GAPDH as a housekeeping gene. The following cycling conditions were used in an Applied Biosystems QuantStudio 6 thermocycler: 55° C. for 10 minutes, 95° C. for 1 minute, and 40 cycles of 95° C. for 10 seconds, followed by 60° C. for 1 minute. The fold change in gene expression for each gene was derived using the 2^{-ΔΔCT}, 2 (Delta Delta CT) method (K. J. Livak, T. D. Schmittgen, Analysis of relative gene expression data using real-time quantitative PCR and the 2(-Delta Delta C(T)) Method. Methods. 25, 402-408 (2001)), normalized to the constitutively expressed housekeeping gene GAPDH. Relative changes were generated comparing the control siRNA knockdown transfected cells to the cells transfected with each siRNA.

sgRNA Selection for Cas9 Knockout Screen

sgRNAs were designed according to Synthego's multi-guide gene knockout (R.

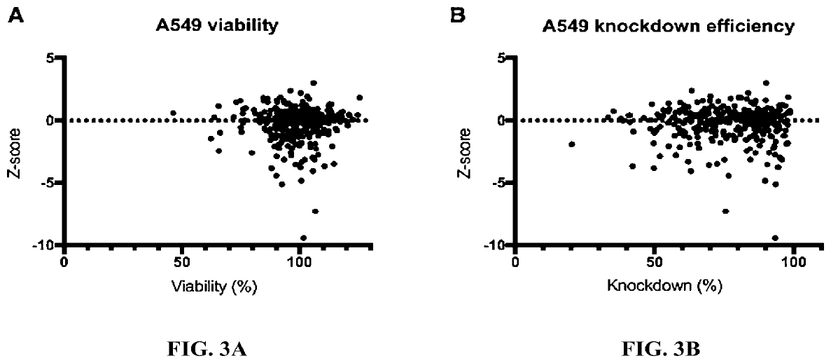

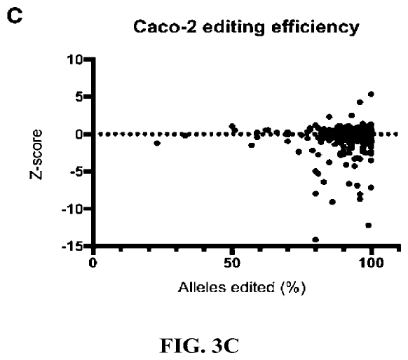

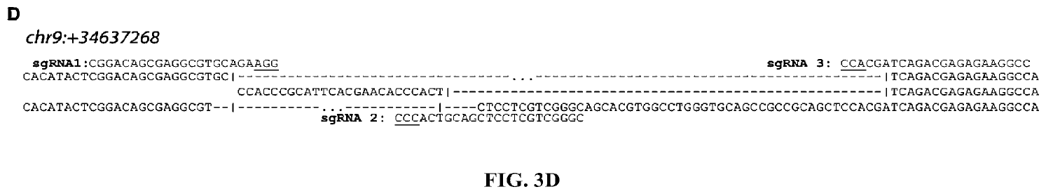

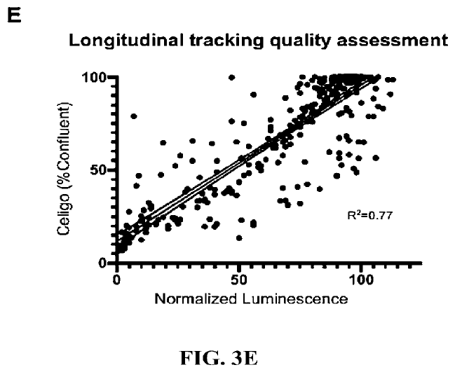

Stoner, et al., Methods and systems for guide ma design and use. US Patent (2019), (available at https://patentimages.storage.googleapis.com/95/c7/43/3d48387ce0f116/US20190382797A1.p df)). Briefly, two or three sgRNAs are bioinformatically designed to work in a cooperative manner to generate small, knockout-causing, fragment deletions in early exons (FIG. 3A-F). These fragment deletions are larger than standard indels generated from single guides. The genomic repair patterns from a multi-guide approach are highly predictable based on the guide-spacing and design constraints to limit off-targets, resulting in a higher probability protein knockout phenotype (see Table 3 provided in U.S. Provisional Application No. 63/091,929 filed on Oct. 15, 2020, expressly incorporated by reference herein).

Referring to FIG. 3A, Z-score was plotted against viability in A549-ACE2 siRNA knockdowns.

Referring to FIG. 3B, Z-score was plotted against siRNA knockdown efficiency in A549-ACE2 cells for 327 of the 332 genes included in the final siRNA dataset. Knockdown efficiency was not obtained for the remaining 5 genes.

Referring to FIG. 3C, Z-score was plotted against editing efficiency (ICE-D score) for 227 of the 288 genes included in the final Caco-2 CRISPR dataset. ICE-D scores were not obtained for the remaining 61 genes.

Referring to FIG. 3D, representative genotype in Caco-2 SIGMAR1 Knockout is shown. Use of multiguide strategy causes genomic dropout between sgRNAs. Plurality of alleles at SIGMAR1 locus have undergone frameshift mutation.

Referring to FIG. 3E, the correlation between quantitative but destructive measurement of cell viability using CellTiter-Glo and non-invasive longitudinal tracking using brightfield imaging is shown. Both measurements are in agreement suggesting both methods can be used to determine gene essentiality (error bars±1 S.D., R2=0.77). These data are from a separate experiment using A549 cells.

Referring to FIG. 3F, longitudinal tracking of Caco-2 gene knockout pools using brightfield imaging is shown. Pools were imaged every day for 11 days except for days of passaging (days 2 and 8, vertical dotted line). The majority of pools showed exponential growth. However, several stayed below the limit of detection (red horizontal line) suggesting pools were lost due to the essential nature of the gene.

sgRNA Synthesis for Cas9 Knockout Screen

RNA oligonucleotides were chemically synthesized on Synthego solid-phase synthesis platform, using CPG solid support containing a universal linker. 5-Benzylthio-1H-tetrazole (BTT, 0.25 M solution in acetonitrile) was used for coupling, (3-((Dimethylamino-methylidene)amino)-3H-1,2,4-dithiazole-3-thione (DDTT, 0.1 M solution in pyridine)) was used for thiolation, dichloroacetic acid (DCA, 3% solution in toluene) was used for detritylation. Modified sgRNA were chemically synthesized to contain 2′-O-methyl analogs and 3′ phosphorothioate nucleotide interlinkages in the terminal three nucleotides at both 5′ and 3′ ends of the RNA molecule. After synthesis, oligonucleotides were subject to a series of deprotection steps, followed by purification by solid phase extraction (SPE). Purified oligonucleotides were analyzed by ESI-MS.

Arrayed Knockout Generation with Cas9-RNPs

For Caco-2 transfection, 10 pmol Streptococcus Pyogenes NLS-Sp.Cas9-NLS (SpCas9) nuclease (Aldevron; 9212) was combined with 30 pmol total synthetic sgRNA (10 pmol each sgRNA, Synthego) to form ribonucleoproteins (RNPs) in 20 μl total volume with SF Buffer (Lonza VSSC-2002) and allowed to complex at room temperature for 10 minutes.

All cells were dissociated into single cells using TrypLE Express (Gibco), resuspended in culture media and counted. 100,000 cells per nucleofection reaction were pelleted by centrifugation at 200×g for 5 minutes. Following centrifugation, cells were resuspended in transfection buffer according to cell type and diluted to 2×10⁴ cells/μl. 5 μl of cell solution was added to preformed RNP solution and gently mixed. Nucleofections were performed on a Lonza HT 384-well nucleofector system (Lonza, #AAU-1001) using program CM-150 for Caco-2 Immediately following nucleofection, each reaction was transferred to a tissue-culture treated 96-well plate containing 100 μl normal culture media and seeded at a density of 50,000 cells/well. Transfected cells were incubated following standard protocols.

Quantification of Arrayed Knockout Efficiency

Two days post-nucleofection, genomic DNA was extracted from cells using DNA QuickExtract (Lucigen, #QE09050). Briefly, cells were lysed by removal of the spent media followed by addition of 40 μl of QuickExtract solution to each well. Once the QuickExtract DNA Extraction Solution was added, the cells were scraped off the plate into the buffer. Following transfer to compatible plates, DNA extract was then incubated at 68° C. for 15 minutes followed by 95° C. for 10 minutes in a thermocycler before being stored for downstream analysis.

Amplicons for indel analysis were generated by PCR amplification with NEBNext polymerase (NEB, #M0541) or AmpliTaq Gold 360 polymerase (Thermo Fisher Scientific, #4398881) according to the manufacturer's protocol. The primers were designed to create amplicons between 400-800 bp, with both primers at least 100 bp distance from any of the sgRNA target sites (Table 4). PCR products were cleaned-up and analyzed by Sanger sequencing (Genewiz). Sanger data files and sgRNA target sequences were input into Inference of CRISPR Edits (ICE) analysis (ice.synthego.com) to determine editing efficiency and to quantify generated indels (T. Hsiau, et al., Inference of CRISPR Edits from Sanger Trace Data (2018), p. 251082). Percentage of alleles edited is expressed as an ice-d score. This score is a measure of how discordant the sanger trace is before vs. after the edit. It is a simple and robust estimate of editing efficiency in a pool, especially suited to highly disruptive editing techniques like multi-guide.

TABLE 4
CAS9 KNOCKOUT AMPLICON PCR AND SEQUENCING PRIMERS

| Gene | | | | Sequencing |
|---|---|---|---|---|
| Symbol | Gene ID | Primer F (5′-3′) | Primer R (5′-3′) | Primer (5′-3′) |
| AAR2 | 25980 | AAGCATCTTTCCCC | TGGGGACAGGTCT | TTCTGATTA |
| | | CACGTT | ACCTCTT | ACTCTGGTTT |
| | | | | CTTTCTTTCT |
| | | | | C |
| AASS | 10157 | GCTGGAGTAAGCA | TCTCAGGAGACCA | GGAGTAAGC |
| | | TAGGGTGA | GAACTGA | ATAGGGTGA |
| | | | | AAATAATAC |
| | | | | TTT |
| AATF | 26574 | TGTTCAGAGTCTAG | TGTCTACTCACCAG | GTATCTTAG |
| | | CTGGGAGT | ACGATCCT | GAAGATCAG |
| | | | | TTGAAGAAA |
| | | | | CTC |
| ACAD9 | 28976 | TGCACGTGACTAA | GGCAGGTTTGGGG | TGCACGTGA |
| | | GGCCTTG | AATCTCA | CTAAGGCCT |
| | | | | TG |
| ACADM | 34 | CCACTTCAGTAGTA | GGAAGAATGGAGT | ATGATTGAA |
| | | TAAATACCACTG | GTGAGTTATTGT | GGCATTTAA |
| | | | | ATAGTGATG |
| | | | | ACT |
| ACSL3 | 2181 | GCCAAGGGTACAC | AGGGACCTGTTTTC | GGTACACAC |
| | | ACAGTGA | CTAACTGA | AGTGAATCT |
| | | | | AATGCTATA |
| | | | | AAA |
| ADAM9 | 8754 | GAGGGCTCAGTTG | GTCCGCACACACCT | GCCGCGCGC |
| | | CGTCAG | GGA | GTGCTCGTC |
| | | | | GGGCGCGCG |
| | | | | TGC |
| ADAMTS1 | 9510 | ACAACGTAGACTC | GGACAGCCTGACC | GTAGACTCC |
| | | CTAAGAGGA | ATAAGCA | TAAGAGGAC |
| | | | | AGTCTCACA |
| | | | | G |
| AES | 166 | CATGACTCACTCCA | CCCTCTTAGAAGCC | CATGACTCA |
| | | GCTGGG | GCAAGT | CTCCAGCTG |
| | | | | GG |
| AGPS | 8540 | AATGTGAAGCTCC | CCTCGACGCTAACT | TGGCACCCG |
| | | AGACGCA | CCTTCC | CCGCCAAGT |
| | | | | CGCCGCGGT |
| | | | | GGC |
| AKAP8 | 10270 | AAAAAGAGAAGCG | ACTATGAGTTCGAC | AAAAAGAGA |
| | | AAGGCGG | CTGGGGT | AGCGAAGGC |
| | | | | GG |
| AKAP8L | 26993 | TTCTGGGAGAAGA | ACATTGAGCCTCCC | TTCTGGGAG |
| | | GGGAGGG | AACCAG | AAGAGGGAG |
| | | | | GG |
| AKAP9 | 10142 | ACGAAGTAGGTTG | CATGCCACTGTGTC | ATAATCTTC |
| | | CCATACCA | CCACTA | CAGGTGGTG |
| | | | | AGTGATGTT |
| | | | | TTA |
| ALG11 | 440138 | TCTCAGGGTAGGTA | AGCGTATCCCATTG | CAGGGTAGG |
| | | GCAGGC | AATCAATGT | TAGCAGGCT |
| | | | | TTTT |
| ALG5 | 29880 | TCCCTCTCTGCCGA | TGAACTAAAACCT | AACTACAAC |
| | | ACTACA | GAGAGTGAGT | AATTATCAA |
| | | | | CTGTGTGCT |
| | | | | CAA |
| ALG8 | 79053 | CTGGCTGAATGGCT | GGCTTCAGAGGGC | GCAGAGGTT |
| | | GTTGGA | TTTCTCC | CTTAACTGC |
| | | | | CTATTAAG |
| ANO6 | 196527 | TCTTCACTTTTAGT | GCTTCTGGTGGCTG | CTTTTAGTG |
| | | GGTGGTCTCT | GATTGA | GTGGTCTCT |
| | | | | GTATTGTTTT |
| | | | | T |
| AP2A2 | 161 | ATGCTGAGAACAC | CTGTGACAGCCTCT | ATGCTGAGA |
| | | TGCTGCT | CCTGG | ACACTGCTG |
| | | | | CT |
| AP2M1 | 1173 | CCACAGGGAGTCA | CTCACCATCCAGCA | TTTAGGCAT |
| | | TAAGAAGGG | GCTCAT | TGGCTTTCTT |
| | | | | TGGAG |
| AP3B1 | 8546 | CACACATTCGCCCC | CGCTCCTCCGTACG | CACACATTC |
| | | AAACTC | AGAAC | GCCCCAAAC |
| | | | | TC |
| ARF6 | 382 | AATCAAGTTGTGCG | CCAGTGTAGTAATG | GATGCCCGA |
| | | GTCGGT | CCGCCA | GTGAGCGGG |
| | | | | GGGCCTGGG |
| | | | | CCT |
| ARL6IP6 | 151188 | CTGCGGCTTCCTTT | CGGGAAAGATACC | ACCCTTGCT |
| | | GCAAC | ATTGCGC | CTCCGTGGT |
| | | | | TTA |
| ATE1 | 11101 | GACTGCACGACTA | TGCCACAATGGAT | GATGGAAAG |
| | | AGTCATCCT | AATAGGAACA | ACCCAGGGT |
| | | | | TTAAAATGA |
| | | | | CTC |
| ATP13A3 | 79572 | CCTCATTTTATCCA | TGTGACAAGACAA | CAGCGATGT |
| | | GGCAGCG | TAAATACCTATCTG | TCCCTTCATC |
| | | | G | TATTATTTC |
| ATP1B1 | 481 | TGGGGTTACCTAAT | TGGCCAGAGTTCA | CTAATCTAA |
| | | CTAAATGCCA | ATCTTTCA | ATGCCAGAG |
| | | | | GAGTGATTT |
| | | | | AAC |
| ATP5L | 10632 | TTGACAGGCTGGAT | CAGGTCAGACGAG | CAAAGATCT |
| | | TCTGCA | TGGAAGG | TTGGACATT |
| | | | | TAAGTATCT |
| | | | | TCG |
| BAG5 | 9529 | GTGTGATACCTTGC | ACCAACATCCTTCT | GAGATTTTT |
| | | TTTCCGC | ATTAGTAGGCT | CCTCCAGTTT |
| | | | | TAACATGTG |
| | | | | TC |
| BCKDK | 10295 | GGTAGATGGGAGC | TTGAGCAGAGAAC | CTGAGCCTG |
| | | TGCTCTC | CCCCAAC | TCAGCATCC |
| | | | | TC |
| BCS1L | 617 | CCTCCACCCTTGCA | AAGTCTCGACACTG | CTTGCATTCC |
| | | TTCCAA | AGGTGC | AATACCACC |
| | | | | CTTAC |
| BZW2 | 28969 | ACAGCAACGTGTG | GCCACACTGCTAG | CGTGTGTAC |
| | | TACATCT | GCCTATT | ATCTATACA |
| | | | | TACATGTCA |
| | | | | TTC |
| C1orf50 | 79078 | GAGGGGGTCCTTG | GCCACCGACTCAC | GAGGGGGTC |
| | | AAAGGC | AATGACA | CTTGAAAGG |
| | | | | CAA |
| CCDC86 | 79080 | TCTCCACCCCTCAC | GTGTAGGTCTTGCT | CTCCACCCC |
| | | CAACAT | GACGCT | TCACCAACA |
| | | | | TG |
| CDK5RAP2 | 55755 | TCAGCTGACAGGG | GACGCTTAATCTCC | TCAGCTGAC |
| | | GACTCAT | TACCTGCA | AGGGGACTC |
| | | | | ATATTTAGA |
| | | | | AG |
| CENPF | 1063 | TGTTAACTTCTTGG | CACCTGTGAAATTA | TGTTAACTTC |
| | | GATTATGGCT | CCTCAAGCA | TTGGGATTA |
| | | | | TGGCTTTAT |
| | | | | AT |
| CEP112 | 201134 | ATTTCCCAGGGCAT | GAAGTTCTGCCTGC | ATATCTAGA |
| | | GCAGTC | CCTACA | TGATGGCCC |
| | | | | TTATTTCTGT |
| | | | | TC |
| CEP135 | 9662 | TGATAACCATGTCT | AGCCAGTATGAAC | CATTGTTTA |
| | | TGTTGAGGT | AGAAACCTT | GTTAAAGAT |
| | | | | CAGGGTGGA |
| | | | | TAT |
| CEP350 | 9857 | GGGAAATCCATGG | CATCATGTTGTCGC | CATCTGGAA |
| | | TGCACCT | CGCTTT | TCAAAGCAC |
| | | | | GTATACTGT |
| | | | | GTA |
| CEP68 | 23177 | AAGCACCTTGATA | ACTCTGGCTGGTCC | GTAGACCTG |
| | | GCCGTGT | TCTTCT | GATAGCTTC |
| | | | | TCTGTCTCTC |
| CHMP2A | 27243 | GGTCCATGCCCAAC | CGTGACCCTGTTCT | TTTTTAACAT |
| | | TCTTGA | GCTTCT | TTGCTGCTCT |
| | | | | GTCTGCTTA |
| | | | | A |
| CHPF | 79586 | TTGCGGCAGCCTTC | GTGCTGACCTCTCA | GGGGCGCAG |
| | | CAG | GACCAC | TTGTTGCAG |
| | | | | CAGCATGCG |
| | | | | CGA |
| CHPF2 | 54480 | CCTTCTCAGCCCCA | TTGGTTGATGCTGA | CTAGAGGGG |
| | | ACTCAC | GGTGGC | GATGTATAT |
| | | | | TCTGAACAA |
| | | | | G |
| CISD3 | 284106 | TCACGGTCCTATGG | TTTTGTTCCCAAGC | GCATCAGAT |
| | | TGTCCT | CCCCTT | CAGCCTCTT |
| | | | | GTAGAG |
| CIT | 11113 | CCGCAAAGCCCTA | AGACGATCTTCTCC | GCAAAGCCC |
| | | ACAGGTA | GCAACA | TAACAGGTA |
| | | | | GACT |
| CLCC1 | 23155 | AATCTTGCTAAATA | ACTTTCAGCATCAG | AATCTTGCT |
| | | CTGACAGTGC | TACTCAATGA | AAATACTGA |
| | | | | CAGTGCATA |
| | | | | TAT |
| CLIP4 | 79745 | AGCACTGATCTGCT | GCATATGAAACAA | TATATTAAC |
| | | GTGTTG | GATGGATTAGAAG | AATAAGAGT |
| | | | GA | GCAGTGATG |
| | | | | AGC |
| CNTRL | 11064 | CACAACCTGAGGC | AGAAGGATGATAT | CTTCGTCAT |
| | | TTCGTCA | CTTAAGGCACA | ATTGCTACT |
| | | | | GAAAACTTT |
| | | | | GTG |
| COL6A1 | 1291 | CGGTTTGGGGTCTC | CTTAGGAGGTTGA | CGGTTTGGG |
| | | TCACTC | GGCCGTC | GTCTCTCACT |
| | | | | C |
| COLGALT1 | 79709 | CTGCAGGTGACGTC | GACTCACCATAGC | CTGCAGGTG |
| | | ACTCC | GCCGTG | ACGTCACTC |
| | | | | CG |
| COQ8B | 79934 | CCAAAGTCACACCT | AGAGGCTGAGGGA | CAAAGTCAC |
| | | ACCCCC | GACTTCA | ACCTACCCC |
| | | | | CAAAGTTG |
| CRTC3 | 64784 | GCCACTTTGTCGGG | AACGGCTAGCGGG | GGGGTCCCT |
| | | CTGA | TGTC | CCAGGTGGC |
| | | | | CGCCGGCGG |
| | | | | CGG |
| CSDE1 | 7812 | CCTTAACAAGGTA | ACATGGGTTTACTA | CCTTAACAA |
| | | AATGCCCATT | TGTGTTCTTCT | GGTAAATGC |
| | | | | CCATTAGG |
| CWC27 | 10283 | AGCAGCTTTCTACA | TGGAATGTTTTTAC | GCAGCTTTC |
| | | AAATAGGGT | AAAGGTAGCTC | TACAAAATA |
| | | | | GGGTATATT |
| | | | | TCT |
| CYB5B | 80777 | GCCACTCCCTTCAT | AAGCCTCCCTTCCT | CCTTCATTG |
| | | TGGTGA | TCCCA | GTGAAAAGA |
| | | | | AAACGAAC |
| CYB5R3 | 1727 | TTACCCCCTCTACA | GCCTCAGAAGAAG | CTCTACAGC |
| | | GCCAGG | CTGCAGA | CAGGGAGAC |
| | | | | TCAGTTC |
| DCAF7 | 10238 | TTTGAAACTAGGG | CAAGAGGGTTCTG | TTTGAAACT |
| | | GTCGGGC | AGGCCTG | AGGGGTCGG |
| | | | | GC |
| DCAKD | 79877 | GTGGAGGGGATGC | AAAGAAGCACCCG | GCCAGTAAG |
| | | CAGTAAG | AGTTCCC | CAGTATGAA |
| | | | | CTCATCAG |
| DDX10 | 1662 | CACAGCCCTCCTTT | CTCCACTCTGCAAC | CCCTCCTTTT |
| | | TCCTGA | TCCTCG | CCTGACGTC |
| | | | | ATT |
| DDX21 | 9188 | CAGTCAAGCAGAT | ATGCTGACTGAGA | CAGTCAAGC |
| | | TCTTTACTATCAGA | GCCCTTG | AGATTCTTT |
| | | | | ACTATCAGA |
| | | | | ATA |
| DNAJC11 | 55735 | AACACACGGCTGG | TCCTGGTGGAGTGT | CTGGGAATG |
| | | GAATGAA | CCTACC | AAGCGCTTT |
| | | | | CTTTTT |
| DNAJC19 | 131118 | GGAAGCAGGAGAA | TGCAGTTTGTAATG | AAGCAATCA |
| | | TGGGTCC | AGTTGGGG | CTTAGAACT |
| | | | | TCATGGATA |
| | | | | TTT |
| DPH5 | 51611 | AGGACAAAGCACC | TGGTTGTCATCGTG | CTTGGTTGG |
| | | CTTTCAT | TTCATCAC | TGTAAAATT |
| | | | | TCCATTCTTC |
| | | | | TG |
| DPY19L1 | 23333 | AGCTCACTCTCCAG | GCACAGCGCCCCT | GGCGGGCGG |
| | | CGG | AAGT | AGGGTGGAG |
| | | | | GGCGGGCTC |
| | | | | GTC |
| ECSIT | 51295 | AGGTCAGAGGGAG | GAGCTTCCTGCAGA | CAAGAAAGA |
| | | GCAAGAA | CGGTG | GAGATGAGT |
| | | | | GATGAAAAG |
| | | | | A |
| EMC1 | 23065 | TGCAAAGGAAACT | CACTAAGCAACAG | CATACTCAC |
| | | CCAGGCA | TGGGTACT | AGCCTTCAA |
| | | | | GATATTCTG |
| | | | | AG |
| ERC1 | 23085 | GTGTGATCTTTTCA | GTGTCATGGTGCTT | GTGTGATCT |
| | | TTACAGATATGGTG | TTAGGTGT | TTTCATTACA |
| | | | | GATATGGTG |
| | | | | TA |
| ERGIC1 | 57222 | GACCCCTACTATGC | TCAGGGTCAGGTC | TTTAGCGGA |
| | | ACTGCC | GAGTGAG | GTCATTGTC |
| | | | | CTGTC |
| ERMP1 | 79956 | AGAGGAGGCCAGC | CGTCTCCCAAAACC | AACAAACTC |
| | | ATTTAAAT | ACCACT | TGTTTTAGTG |
| | | | | AGTCAATGT |
| | | | | AT |
| ERP44 | 23071 | CAGTATAACATAA | TGAACCAAAAAGT | ACTCATTAA |
| | | GCATTTGCCTTGAG | TCTCACTAAGCA | GTATACGTA |
| | | | | TGTCAAATC |
| | | | | CAC |
| ETFA | 2108 | AGGGAAGAAACCT | GACACAAATAGCT | CTTTTAGTTC |
| | | TTTAGTTCCT | AGATTTTCGCT | CTTTTTCACA |
| | | | | CATGGTAAT |
| | | | | G |
| EXOSC2 | 23404 | CCCTTCGGGTTCGC | TCCAGGTCTCCCAC | GCCTTATTGT |
| | | CTTATT | AAGGAA | TGCCAATTG |
| | | | | TAAACATG |
| EXOSC3 | 51010 | TCAAAGCAGGGCT | AAGGGCGGGTGTT | CAAAGCAGG |
| | | ACCACTC | GGAAG | GCTACCACT |
| | | | | CTC |
| EXOSC5 | 56915 | AGTCGTGAGGGAG | ACTGGTTACGCAGC | GTATCCCTG |
| | | AGATGTGT | CTGTTT | CGTATTTAG |
| | | | | TAGTATTCA |
| | | | | ATC |
| EXOSC8 | 11340 | GGCCACAGTTGCCT | TCCTCTTACCTTTC | CAGTAATCC |
| | | TTACTG | CTGGAGA | ATAAATTGA |
| | | | | AAAGTTTAG |
| | | | | GCC |
| FAM134C | 162427 | GTGCAGCGAAGAA | CATCTGCGCAGTTG | GAGAAGTAG |
| | | AACAGGG | CTGTTA | AGCCCTAGA |
| | | | | GGAACCAAC |
| FAM162A | 26355 | AAGACACATGTGG | GGGTATGATATAG | AAGACACAT |
| | | GAAGTACTT | GAACCTCTTCTCT | GTGGGAAGT |
| | | | | ACTTATTTA |
| | | | | AAA |
| FAM8A1 | 51439 | ACCAGCCACCGAC | CACTTGCCGGGAGT | ACCAGCCAC |
| | | TACTAGG | ACTCG | CGACTACTA |
| | | | | GG |
| FAM98A | 25940 | ACGTCTACCCTCAG | TGCAGTGGTGTAA | GTCTACCCT |
| | | CTCCTA | GAAAGGAT | CAGCTCCTA |
| | | | | AATTGG |
| FAR2 | 55711 | AAAGCCACGATGC | CATTGCCCATCACA | AAAGCTCTT |
| | | TCTCACT | CACGC | GGAAGCAAC |
| | | | | AGAAACATT |
| | | | | TTA |
| FASTKD5 | 60493 | ACAGACAGGAGCT | GCCAAAGAGATCA | GAGAAGTCT |
| | | GAGAAGTC | ATACTGACACC | CAGATGCAT |
| | | | | TATAGCTGT |
| | | | | GAA |
| FBLN5 | 10516 | TTGTGGTGAGCATG | GGTGTTTGGGAGTG | GTGAGCATG |
| | | CCAGAT | CTTCCT | CCAGATACA |
| | | | | GACGATG |
| FBN1 | 2200 | ACAACCCTAGCAC | TGGAGAAGGCGGG | GAGGTCTTG |
| | | CTCTAAGG | AGGA | CCAAGGAGT |
| | | | | CTTC |
| FBN2 | 2201 | GCTCCAGCTAAAG | CTGACTCTTTTCTG | CTCCAGCTA |
| | | GGTCTGG | AGGCGC | AAGGGTCTG |
| | | | | GGA |
| FBXL12 | 54850 | GTCACACGGTAGG | CCTCTCACTCTGTC | GTCACACGG |
| | | TACCACC | ACCCCA | TAGGTACCA |
| | | | | CC |
| FGFR1OP | 11116 | CGTTGAAGGTAGA | TGCATTGATACAAT | GGCTCTGTA |
| | | GGCTCTGT | CTGAATGCATC | AAAGAAATA |
| | | | | GGCATAATT |
| | | | | TTT |
| FYCO1 | 79443 | ACTCTGCTAGCTCC | CACGGGACTCACT | ACTCTGCTA |
| | | TCCTCC | GGACAAG | GCTCCTCCTC |
| | | | | C |
| G3BP2 | 9908 | GCACATGTACACA | TGTAAGGAAATCA | TCACTCAAA |
| | | CACGCAC | ATGAGGGTAGGT | CAACAGGTC |
| | | | | AAACACAAA |
| | | | | TTC |
| GCC1 | 79571 | CTGCTACTGCTAAC | CGTTCAGACCCTCC | CTCTTCGGA |
| | | GCCACT | ATGGAG | CTTTGGAGG |
| | | | | TGG |
| GCC2 | 9648 | TGGGAGATGCACA | TCTCTGCTTCATGT | GAAAATTTG |
| | | TAAGGAGT | TCCTTAGCT | AAAAATGAG |
| | | | | TTGATGGCA |
| | | | | GTA |
| GDF15 | 9518 | TCCCCCTAAATACA | GTGAGTATCCGGA | CTAAATACA |
| | | CCCCCA | CTGCAGG | CCCCCAGAC |
| | | | | CCC |
| GFER | 2671 | CGCCACACACTGCT | TCCGCATCCACGTC | CCACACACT |
| | | CTTTTAC | TTGAAG | GCTCTTTTAC |
| | | | | TGGAGAAAG |
| GGCX | 2677 | ACAGCATGAAATT | AGCTGTCAAGACC | CAGCATGAA |
| | | GATCACAGCA | CTAACAGT | ATTGATCAC |
| | | | | AGCAGAAGT |
| | | | | GAA |
| GGH | 8836 | TGGTCATTCACATC | TCCATGTGTAACTC | TCATTCACA |
| | | TTCAACCTG | AGGTGCC | TCTTCAACCT |
| | | | | GTGTAAATA |
| | | | | AT |
| GHITM | 27069 | ACAGTGGATGGTT | CACACTAAAGGCA | CTGAAAATT |
| | | GGGCAAA | GAGCAGC | AAAAAGGTC |
| | | | | GCTTTATTTC |
| | | | | CT |
| GIGYF2 | 26058 | TGTTCTCTTTACTA | ACAGCAGATTTGG | CTCTTTACTA |
| | | GGTCAGTCCA | CTTTGGT | GGTCAGTCC |
| | | | | ATTTGAGTTT |
| | | | | G |
| GNB1 | 2782 | ACTGAAAGAGACA | TGGGAAGAGGTAG | AAGGGAGAA |
| | | GGAGAAGGG | GCACAGT | AGAAAAATC |
| | | | | AGAACTTGT |
| | | | | ATT |
| GNG5 | 2787 | GAAAGTCCTGGGG | AGAGACAAAGTTC | GAACTAATC |
| | | CGGAAG | GGAGCCC | GTCCCCCTA |
| | | | | AAACACAG |
| GOLGA2 | 2801 | ACAGTGCCCCCAA | AAGAAGGTGGGAA | AAACTCACC |
| | | ACTCAC | TCTGGGC | CACAGCAGC |
| | | | | TG |
| GOLGA3 | 2802 | GACGTGGAGGGTG | AGAAAGTGCCGTG | CTTACACAG |
| | | GGAAAAG | CTCATGA | TTGCGTTTCT |
| | | | | TGCATAGGA |
| | | | | AG |
| GOLGA7 | 51125 | TGAGCCTTGAAGCT | ACGGCAACTATCA | TTGAAGCTA |
| | | ATCCAGT | CCATGTAA | TCCAGTATTT |
| | | | | ATAAGAGGG |
| | | | | AT |
| GOLGB1 | 2804 | ACAAGCCACTCAG | GCAGTTACAGCAG | CTCAGATGG |
| | | ATGGTAGAG | ATGGAAGC | TAGAGATGT |
| | | | | GGACTTC |
| GORASP1 | 64689 | CATCCTGCCCTCAG | CCATCTCAGGCCCA | CAGACCTGC |
| | | TCTTCC | GACTTG | CCCAGTAAA |
| | | | | CTCATC |
| GPAA1 | 8733 | GTATCAGGCCCAG | GAAGGGAGCCTCT | GTATCAGGC |
| | | GCTTAGG | GAGCAGA | CCAGGCTTA |
| | | | | GG |
| GPX1 | 2876 | ACAGGAGAGAAGG | TATCGAGAATGTG | TTCTAACCA |
| | | GCAGCTA | GCGTCCC | CAAACAAGG |
| | | | | GAGATTTTC |
| | | | | TAT |
| GRIPAP1 | 56850 | CCGGCCGCAAATA | ACTTGGTATGCTCT | TGAACTAAT |
| | | TCTCCTT | TGGTATTCT | GCAGAATGA |
| | | | | TATCACCTTT |
| | | | | TA |
| GRPEL1 | 80273 | GCAGTAACTTGCCA | ACAGAAATGTTTTC | CTTAACTCT |
| | | CCTGGG | TCCCCAAGT | GGCTTTAGT |
| | | | | CTGTCACCA |
| | | | | ATG |
| GTF2F2 | 2963 | CGGCGTGTTCCTCT | GGCTGAAAGACAC | TGTTCCTCTT |
| | | TTTCCT | TTTGCGT | TTCCTCGGTT |
| | | | | CC |
| HEATR3 | 55027 | TATGCCCTCTTCCA | GTCAGAAGGCGCG | TATGCCCTCT |
| | | CGCCT | CAATG | TCCACGCCT |
| | | | | G |
| HECTD1 | 25831 | GCTCCGACCTCAGA | CTTTGCTGCAGTTG | AGAAAGAGA |
| | | AGATCC | CCTTTCT | ATGGGAAGA |
| | | | | AAGATGTTT |
| | | | | AAT |
| HMOX1 | 3162 | CTGCTTGTTTTGCC | CAGGGCTTTCTGGG | TAAAAGGTT |
| | | CAGTGG | CAATCT | TTTAGGCTG |
| | | | | AGAAAGTGC |
| | | | | ATG |
| HOOK1 | 51361 | AGTGCTTTTGGTTG | GCTTTCTGCCAAGC | CTTTTGGTTG |
| | | GTTACTCA | TTTAATAGT | GTTACTCAG |
| | | | | AATTTTGGA |
| | | | | AT |
| HS2ST1 | 9653 | CCCATGTTTTCCAT | TGAGATCAGCACTC | TTGTATCTTT |
| | | ATCCCTTGG | ACATCCC | TCTAATCAT |
| | | | | GGTCCAAAG |
| | | | | TT |
| HS6ST2 | 90161 | CGAACGTGCGCTA | CACAGAATGCCAG | CCCAGCACC |
| | | CTGG | CTCCTCC | TGCCCAGCC |
| | | | | GGGGTGCAA |
| | | | | ACG |
| HSBP1 | 3281 | CTACTCCCATAATG | GCGACAGATGAAT | GGTCCCGCG |
| | | CCCCGC | GGGGCTA | AGCTGCCAG |
| | | | | TCTCGTCGC |
| | | | | GAG |
| IDE | 3416 | GACTCTGGACCAG | AAAACCCGGAGCA | GCTCCCGCC |
| | | GCCTCT | GCTACC | TGGCGAGCC |
| | | | | GCTCTTCCG |
| | | | | GGC |
| IL17RA | 23765 | TCTGGGTCGACAG | CCTGAGTCGCGAG | CCATGCATG |
| | | ACTGTGA | CTTCTAG | AGCTCAGGT |
| | | | | AACAG |
| INHBE | 83729 | TGTGGCAGGAGAA | ACACCAGACTTCTC | GACACAAAG |
| | | GGAGGAG | ACCCCT | CAGTCTCTA |
| | | | | CTTTTCTAGA |
| | | | | G |
| INTS4 | 92105 | CTCAATAAAAGCTT | ACTGTATTTTCCTA | CTCAATAAA |
| | | CCTAATGAATACCC | AGTCCATCAGCT | AGCTTCCTA |
| | | | | ATGAATACC |
| | | | | CTA |
| ITGB1 | 3688 | CGAGCCTTCAACA | AAAGCCAGAATTG | CAACAGAAA |
| | | GAAACTGG | GGGTACA | CTGGTCAGA |
| | | | | GTTTGCATA |
| | | | | AAG |
| KDELC1 | 79070 | ACCAGGACTCATA | AGAGGAAGAATGT | GTAGAAAGC |
| | | ACTTAGCTTTCA | GGAGGAGA | CTTTATTTTT |
| | | | | CTTCTTTCAG |
| | | | | T |
| KDELC2 | 143888 | GGAGCTGACCAGA | TTTCCCGCCCGAAA | AGGGGCGAC |
| | | CCCAAAA | GACC | ACACGCCGG |
| | | | | GGAGGGACG |
| | | | | CCA |
| LARP4B | 23185 | ACTTGCAGTGACTC | GCTGAGCCTTTGGA | GTGACTCAA |
| | | AACTTCT | GCCTAT | CTTCTTTAGA |
| | | | | CTGTAAAAG |
| | | | | AC |
| LMAN2 | 10960 | GTGACCTTCCTTCC | AGCTGGGGAGAGA | GACTTTATC |
| | | AGAGCC | AGAGAGG | CACGGGAGG |
| | | | | CAG |
| MAP7D1 | 55700 | ACAAGGGAGAGGG | CGGGGTCATTACAC | ATGAGCAAT |
| | | CCACATA | ACACCT | CTGACCTCT |
| | | | | CTCCTCTCTT |
| MARC1 | 64757 | CCGACCAAGTGGA | CCCCCTTGCAGGAT | CCGACCAAG |
| | | AGCTGAG | TTCACA | TGGAAGCTG |
| | | | | AG |
| MARK1 | 4139 | AGGGAGCTGAAGT | AGACTCCAGAGAG | GGAGCTGAA |
| | | CCAGAAGA | GTCCAGG | GTCCAGAAG |
| | | | | AAATTATAA |
| | | | | ATA |
| MAT2B | 27430 | CTGATGCCCGACCC | CTTGAGAGCAAGG | TAACTTAAC |
| | | TAACTT | ATAGTTTCTGT | TTTAGAATT |
| | | | | GGCTTGCAG |
| | | | | ATA |
| MDN1 | 23195 | ACTTCCAAAAATG | TCTTTTCTGGGGGT | CCAAAAATG |
| | | AAGCAGCAA | GACAGG | AAGCAGCAA |
| | | | | TTTAACAAA |
| | | | | CTA |
| MEPCE | 56257 | GCGGTTGAGTCCTC | TTTCCCGACCGACC | CGGTTGAGT |
| | | GAGTAG | GCA | CCTCGAGTA |
| | | | | GTTC |
| MFGE8 | 4240 | CAGACAGCAAACA | CCTCCCAGGTCTGA | CTGCCCTAC |
| | | CCTGGGT | AGAGGA | CTAGCTCAG |
| | | | | TTTG |
| MIB1 | 57534 | GTTATTCTCACGTC | GTGTCCCACTGCAG | GGCTCGCTG |
| | | CCCCGG | ACCTC | CCGCCCCCG |
| | | | | CCGACGCCT |
| | | | | AGA |
| MIPOL1 | 145282 | GTCCCAGCCGTCAC | ACCCTGATGGCAA | CTCCAAAAT |
| | | TAAATT | GGTATGG | TTACCTGTG |
| | | | | CTTACAAAT |
| | | | | TTA |
| MOV10 | 4343 | TCCTTCAGGGAATG | CCTGTCCACCAGCT | GAGGGGGTG |
| | | GGGGAA | CTTTCC | AGTTTCCTA |
| | | | | AGC |
| MPHOSPH10 | 10199 | ATGTTGTTGGGGGC | CCATGTCGGACACT | AAGAGTGCT |
| | | CAGAAT | TCCTCC | GTGAAATTA |
| | | | | TTACCTGTA |
| | | | | ATT |
| MTCH1 | 23787 | AGCCTCCCATCTCC | ACATCCGGCGTGTC | AGCCTCCCA |
| | | CTACG | CCA | TCTCCCTAC |
| | | | | GG |
| MYCBP2 | 23077 | CACACACGAGAAA | GACGGATTCTACCC | CTCCTATCTC |
| | | CTGCAGC | AGCCG | GATAAGTGC |
| | | | | TCCTG |
| NARS2 | 79731 | TGAAAGCAAAGTT | GAGCAGCTGAGAA | GTTATCGGA |
| | | CCAGCGC | AGGAGGG | ACAGTTTTG |
| | | | | TGAAAAGTA |
| | | | | ATG |
| NAT14 | 57106 | GTGTGCCACACTGA | TCCCCTGCATTTGT | CCACACTGA |
| | | ACATCG | GCCAG | ACATCGGAC |
| | | | | TGT |
| NDFIP2 | 54602 | GACCTTCCTCTTTA | AGCCCATTAACAG | ACCTTCCTCT |
| | | TTGTAAAGAAACT | ACATGATAATTACA | TTATTGTAA |
| | | G | C | AGAAACTGA |
| | | | | AA |
| NEU1 | 4758 | GGTTCCCTCTACCC | CTTGTTCTGGGACC | CTCAGGCAA |
| | | CTCAGG | CCATCC | CCAACCCTC |
| | | | | TAAGTTC |
| NGDN | 25983 | TCTGAGCGTTGTTT | TCACTTAAATGAGA | TCTCTTGTAT |
| | | CTCTTGT | GCTACTGTGTGA | TAGCATAAC |
| | | | | TTTCTCATTG |
| | | | | G |
| NINL | 22981 | CTCCCCAAAGTGAC | GCTGAGTGTGCACC | CCCATATCTT |
| | | CAAGCT | TTCTCA | GTGATTATG |
| | | | | TGCTACAAA |
| | | | | AA |
| NLRX1 | 79671 | AGTTTGTCCAGTGG | GGCATCCGGGTTA | CAGACTTTC |
| | | CTTCCC | AAGAGCT | TGGACAGTC |
| | | | | TATATTTTCT |
| | | | | CA |
| NOL10 | 79954 | GCAAAGCTCACTG | CCAGGAAGTGCGT | AAAGCTCAC |
| | | ACCCTGA | CATCAGT | TGACCCTGA |
| | | | | TTATCC |
| NPC2 | 10577 | TAAAGGGAGTCTG | GAGCAGAGCACCT | AGTGAACCC |
| | | GGAGCCA | TCCCATT | TAGCTTTGC |
| | | | | ATGAG |
| NPTX1 | 4884 | GGTCGCCCATGGTG | ATAAAAGGCGCGG | CCCGAGCCG |
| | | TTCTT | GCTCC | GGCTGCTTG |
| | | | | CGGCCGCCG |
| | | | | CCC |
| NSD2 | 7468 | AGCTGTAGAGGTC | GGGTGTCCCAATCC | ACCTATCCT |
| | | CTGGCAT | CTTTCA | AGGTTTTAA |
| | | | | ATGTAATTG |
| | | | | CTT |
| NUTF2 | 10204 | AGGGAAACTGAAG | CAAGACTCTCCTCT | CTTTTCAGA |
| | | TGTGGCC | GCCTGC | GTCTTTCCA |
| | | | | GGGCCTTA |
| PABPC1 | 26986 | GCGCGTCATCACCC | CATGGCCTCGCTCT | GTCATCACC |
| | | TAAAGT | ACGTG | CTAAAGTTT |
| | | | | GAGAGC |
| PABPC4 | 8761 | TGGCAACATGCTGT | ACTCCAGCTCGTCC | CAACATGCT |
| | | CGTGAT | TCG | GTCGTGATG |
| | | | | CC |
| PCNT | 5116 | GGGAGAGCATGTG | GGACTTGGATCGA | GTGTGGTCT |
| | | AGCACG | ACCCAGG | CATGAACCT |
| | | | | AGTGAG |
| PCSK6 | 5046 | TCAGACTCCCCGAG | CTGTGATGCGGTGT | CGAGTGACT |
| | | TGACTC | CCTCAT | CCTCCACAC |
| | | | | TG |
| PDE4DIP | 9659 | CGAATCCCTTGGCC | ACCATCAACTAACC | ATATCCCAC |
| | | AGTGAT | CTCCACA | TTGAAAGTA |
| | | | | TAGGCAGAA |
| | | | | TAT |
| PDZD11 | 51248 | CCGCGCTGAACCTC | GGTTGGAGCTGCTG | CTGAACCTC |
| | | TTAACA | TCTGAA | TTAACAGTA |
| | | | | TGGAAATGA |
| | | | | AG |
| PIGO | 84720 | TGGGGCTGAATCTC | GCTGGGCTTGTATT | GAATCTCCA |
| | | CAGGAT | CAGGGA | GGATCCTCT |
| | | | | GCAAG |
| PIGS | 94005 | GTGAAGGGCAGCT | CTTCGCACGGAGAT | CACTGACTC |
| | | TCTCCTG | CCCAAT | CCGCGTAAA |
| | | | | CA |
| PITRM1 | 10531 | CCATGTGGCTTTCC | GCTGGAGGATTGT | TCCTGAAGG |
| | | TGAAGG | GGTGTCA | ATTAAATTT |
| | | | | CTAATGTCC |
| | | | | TTC |
| PKP2 | 5318 | TCTCTGGAAGCCCT | TCACGTACCCCAGG | CTCTGGAAG |
| | | TCTCTCA | CCA | CCCTTCTCTC |
| | | | | AAG |
| PLD3 | 23646 | TGAATAGCCCCAA | TTTCTGTGGGGAGG | GAATAGCCC |
| | | GACTAATCACT | AGGAGG | CAAGACTAA |
| | | | | TCACTCTTCT |
| | | | | G |
| PLEKHA5 | 54477 | ACATTCCCAACCAT | TTCATGACCCCTCC | AACCATAGA |
| | | AGAGTGCT | CCTTCT | GTGCTAATT |
| | | | | AAACCAGAG |
| | | | | ATC |
| PLEKHF2 | 79666 | GCCCTTTTGATGTG | AGTGACATTTTCCA | TTTTGATGTG |
| | | CTTAGTGA | GGGGAAT | CTTAGTGAT |
| | | | | TATCTTAGA |
| | | | | GG |
| PMPCA | 23203 | ATAAATACGCACG | CGTTCCCGCTACTT | CCAGAGTGC |
| | | CAGCTGC | CACCTT | AAGTAAAAT |
| | | | | ATCAGCTTG |
| PMPCB | 9512 | TGGCTTTAGGACAG | CACCAGCCAACGA | CAGAGATCT |
| | | TGGCTG | AAAAGCT | CAGTGGAAC |
| | | | | CAAAATTCA |
| | | | | A |
| POFUT1 | 23509 | AGCTTTGGCGTCTT | TGACATAGTCTTGG | TTTTAATTGT |
| | | TTGATGA | GGGCCT | CATGTAGTC |
| | | | | TGAACTGTC |
| | | | | TT |
| POLA1 | 5422 | CCCAATTTGGAGAT | CCTCTGCAGAAATC | CAATTTGGA |
| | | TAAAGAGAAATGC | ACATTTTCA | GATTAAAGA |
| | | | | GAAATGCAA |
| | | | | ACA |
| POLA2 | 23649 | AGGTCTGGGTATGT | TGGAACTTGTTCTA | TCCAACCCC |
| | | CCAACC | CCAGCCT | ATTAAACTG |
| | | | | ATTCAATTT |
| | | | | ATA |
| POR | 5447 | GTCCAAGACTGTG | GGACAGAGAGAGG | GTCCAAGAC |
| | | GCTGTCT | AGGCTGA | TGTGGCTGT |
| | | | | CT |
| PPIL3 | 53938 | TCACATTTTAGGGG | TGCTGCTATCACGT | GTGCTAATA |
| | | TAGGTGCT | TTTCAGT | ATTTCTGCTT |
| | | | | TAAAATTGC |
| | | | | AC |
| PPT1 | 5538 | GGCTCCTTCCCCTT | CTGAAAGCTCCAG | CTTTCCAAT |
| | | CTCTCT | GGTAGGG | GCAGATCCT |
| | | | | TCAAATCCT |
| | | | | AAA |
| PRIM1 | 5557 | TAATGTGAGCCTGA | TCGGCCATAAGCG | TAATGTGAG |
| | | CCACGC | CCTG | CCTGACCAC |
| | | | | GC |
| PRIM2 | 5558 | GGATATTTTCTGCA | GAGGTTGAGAAAC | TATATGATG |
| | | CATAGATGGACA | CCTGCCA | TCGTTACAG |
| | | | | GAAATAAAC |
| | | | | TGG |
| PRKAR2A | 5576 | TGCCACCCCTCTAG | GAAAGGCCGGCGT | CCACCCCTC |
| | | ACCTC | GAGT | TAGACCTCT |
| | | | | GG |
| PRKAR2B | 5577 | GAGGTTGCCATGGT | CTCACCATTGAACG | GAGGTTGCC |
| | | TTCCGG | CCCCT | ATGGTTTCC |
| | | | | GG |
| PRRC2B | 84726 | GAAGGGGCATGAT | AGTGGCATCAGCA | CACAGAGCA |
| | | GCTGTCA | CCCTTTT | CCCTTGTGA |
| | | | | CAAG |
| PSMD8 | 5714 | CCCGAGCACTCAG | TTGCTCGTACATGC | CAGGGCAGC |
| | | ACTGAAG | CGGTC | CATGTTCATT |
| | | | | ATTG |
| PTBP2 | 58155 | ACATTGATCCCAAA | TCACCATACTGGAG | TTAAAAATA |
| | | GCCTGG | CAAAGCT | TCTGTTGAG |
| | | | | GGGCCATTT |
| | | | | AAT |
| PVR | 5817 | TACCCTCCTCGCCT | AACCCGAACATCCT | TACCCTCCTC |
| | | GCCAT | CAGCG | GCCTGCCAT |
| | | | | G |
| QSOX2 | 169714 | CACTCGGGAAATG | CTCAGAAACCCAC | GAAATGGGT |
| | | GGTGGAA | CCCAGC | GGAATGAGT |
| | | | | TGGG |
| RAB10 | 10890 | TGTCACTTCCTACT | AGTACATTATATCC | GTTTTCCCTT |
| | | GTTTTCCCT | TGAAGATCAGTTG | TCAGATTTTC |
| | | | G | ATCCAGTAT |
| | | | | G |
| RAB14 | 51552 | GTTTTACATGGCAA | TGCTTATTTAGTGG | GTTTTACAT |
| | | CTTAAGAAACC | ATTTTCCCCC | GGCAACTTA |
| | | | | AGAAACCAT |
| | | | | AAA |
| RAB18 | 22931 | AGCTGGAGTTTAG | CTCATTGACATGTG | CCATGGGTT |
| | | AACCATGGG | TTTTCAAACCA | TCATTTCATG |
| | | | | TATGATAAA |
| | | | | AG |
| RABIA | 5861 | CAACCAGAATCCCT | TCACATCCTGATAA | CCTTGAAAG |
| | | TGAAAGCA | TCTCCACAGT | CAAACGTAA |
| | | | | AACTAATTA |
| | | | | CTA |
| RAB2A | 5862 | TGTGCGTCTCGTTG | ACAATTCAGTTGCA | TAACTTTTTC |
| | | ACTTGA | GGTTTCTGT | CTAAGACTG |
| | | | | GTGAAGTTA |
| | | | | AG |
| RAB5C | 5878 | AGTTGCTGGGCTCA | TTACAGTTGGAGGT | CACAGACGC |
| | | ATTCCA | CCCCCT | ATTTAGTCC |
| | | | | CTAATG |
| RAB7A | 7879 | TTCCACATCTGCCC | TGAAGAACAGGGA | GTACCCTAT |
| | | CACATC | AGGAAAATGT | ATTTTTACCC |
| | | | | AGAGAGAAA |
| | | | | AC |
| RAB8A | 4218 | AAGGTCTCCCCGCG | GCGACTGCTCTTCT | GGGACGCAG |
| | | ACT | CCCTTT | GGGCGGGCG |
| | | | | TCGGCCGCG |
| | | | | GTG |
| RALA | 5898 | TGTTTGCAAATGAG | TGTCACAAGCAAC | CAAATGAGG |
| | | GAAACCAAGA | AACATTACTCT | AAACCAAGA |
| | | | | AATTGTCTA |
| | | | | AAA |
| RAP1GDS1 | 5910 | TGTGGAGCAGAAG | TGGGACAGGTATG | GGAGCAGAA |
| | | GTAATTTTGT | AATGACTGT | GGTAATTTT |
| | | | | GTATAAAGA |
| | | | | CAT |
| RBM28 | 55131 | CGTAAGGGAATGC | ACGAGCACTTCCG | ATAAACTGA |
| | | TTTGCCC | GAATCTC | CTCCTATGA |
| | | | | ACGCATCTA |
| | | | | AAG |
| RBM41 | 55285 | GCTTCTCTTTTACC | CTCTTACAGTGCTG | CAATGTCTG |
| | | AATGTCTGCA | AACCTCCA | CATTCTAAA |
| | | | | AATCAAAGA |
| | | | | AGA |
| RDX | 5962 | CCATGATCCAGCTG | CAGAGACTCTTCTT | ATCCAGCTG |
| | | GCAACT | CTTGCAAGT | GCAACTTAA |
| | | | | AATCTGGAA |
| | | | | AAA |
| REEP5 | 7905 | TCCGATGCCCACGC | GAGTGGAGGACGC | CTGATCCCT |
| | | TTTC | GTAGAC | GAATATGCT |
| | | | | GCTTGTC |
| REEP6 | 92840 | GGAGCCGTCACTCT | TCTCCTGGTATCCT | GTCACTCTG |
| | | GCTAAG | CCGGAC | CTAAGCCTG |
| | | | | TATCTG |
| RHOA | 387 | ACTTGGACTAAGAT | GCCCCATGGTTACC | GAATGGATT |
| | | GGCAGGA | AAAGCA | CTTCTTTCCA |
| | | | | ACATTTTTGT |
| | | | | T |
| RNF41 | 10193 | GCTCCAATCTGATT | ACAAGAGGGAGGC | CACAGGCAG |
| | | CCCTGCT | CTGAAATG | AATATCCAC |
| | | | | TCATCTAG |
| RPL36 | 25873 | AGCAGGTAAGTGG | CAGGCAGGAAGTC | AGCAGGTAA |
| | | TTTCCCG | CCACTC | GTGGTTTCC |
| | | | | CG |
| RRP9 | 9136 | TAGTGTTGGCCTTT | TCCTGCATTATCCA | CAAGGCTAC |
| | | CCCACC | GCCCTG | AACAACCAG |
| | | | | ATCCTTA |
| RTN4 | 57142 | AGTCTCCTCCATCA | AGAGTGGGTTTAA | GTATAGCTC |
| | | TGAGCCT | AATGTGGGT | AAGCAAATA |
| | | | | ACTGCAATT |
| | | | | ATC |
| SAAL1 | 113174 | ATAGTTTTGGGGTC | CAGGCTCCGAACA | ATAGTTTTG |
| | | CGCAGC | GCAGATG | GGGTCCGCA |
| | | | | GC |
| SBNO1 | 55206 | GCTTCACATGTATA | TGGGTCTAATAGA | CTTCACATG |
| | | TTTAAAATTGGGCC | GATTGTTGGATTGT | TATATTTAA |
| | | | | AATTGGGCC |
| | | | | AAG |
| SCAP | 22937 | TTAGCTAACCAGGC | CCTAGTGTGCAGA | TTAGCTAAC |
| | | CAGGAC | GCCAAGT | CAGGCCAGG |
| | | | | ACTAGAGTT |
| SCARB1 | 949 | AAACCAAGACAGG | ATTGCAGGCGAGT | AAACCAAGA |
| | | TGGACCC | AGAAGGG | CAGGTGGAC |
| | | | | CC |
| SCCPDH | 51097 | TAGGAAACCTCCC | GAAACGCTCGTTTG | TAGGAAACC |
| | | GTCGGAA | GGGC | TCCCGTCGG |
| | | | | AAG |
| SELENOS | 55829 | GCCCCACCGAGAA | GGCTTTGAGGGCA | CACCGAGAA |
| | | CCATATA | GGAGTTA | CCATATACT |
| | | | | TCCTACTTTT |
| | | | | T |
| SEPSECS | 51091 | CACCCCCTCCTAAC | GCGAGTTGCATTCT | CTCCTAACA |
| | | AACACC | GGTTCC | ACACCATTT |
| | | | | GGCTTTCAC |
| | | | | TG |
| SIRT5 | 23408 | GCATCTGCCATGTT | CTGAAACAGCAGG | CATCTGCCA |
| | | GTTTGA | ACAGGTG | TGTTGTTTGA |
| | | | | ACATAGT |
| SLC25A21 | 89874 | AAGGGAAAGCACT | ATTCTGGCTTGAAG | TTCTTCAAG |
| | | CAGGTGT | GGAAGTT | AAGATAAAT |
| | | | | TTTGGTGTC |
| | | | | AGA |
| SLC27A2 | 11001 | GTGGCAGGAAAAA | ACTGGCTACGTATG | AGGGGCATT |
| | | GGCAGAC | CTCTCA | TATAACCAA |
| | | | | CATAAATAT |
| | | | | GTA |
| SLC30A6 | 55676 | TCAGTTCAAGTTGC | ACAACTTAACACC | GCCTTATCC |
| | | CTTATCCA | AAACAACTGCA | ATTTAAAAA |
| | | | | TAAAGAGTG |
| | | | | TGG |
| SLC30A7 | 148867 | GTCCGGCAGAAAG | GCAACTCAGCAGC | GTCCAGTGA |
| | | GGAGAAG | AGAGGTA | GGGAGAGTC |
| | | | | AAAAACTC |
| SLC30A9 | 10463 | AGGAAGGCCTCCC | GAAGGTTCTGAGG | CCTATTGGT |
| | | TATTGGT | TTGGCGA | GCTCAACGT |
| | | | | GTTAC |
| SLC44A2 | 57153 | CCCCTGGTTCTGCT | GTTTGCTGGGGATG | CTGGTTCTG |
| | | GGAATT | AGGACA | CTGGAATTC |
| | | | | CAATG |
| SLC9A3R1 | 9368 | GGGATTGGTCTGTG | CCTGCTGGTGGGTC | GATTGGTCT |
| | | GTCCTC | TCCTT | GTGGTCCTC |
| | | | | TCTC |
| SLU7 | 10569 | GGGGGACAAGAGA | CCTTGAGGAGGGG | TGTAGGTAT |
| | | GGAAGGA | GAAGAGA | TATTATCTA |
| | | | | GAGATGTGA |
| | | | | CGG |
| SMOC1 | 64093 | TGCAGCAGTTACTA | GGGGAGTTGAAGA | TTACTAGCC |
| | | GCCACG | GCCACTC | ACGGCCCTT |
| | | | | TTAG |
| SNIP1 | 79753 | CACTCTCAACAGCC | GAAGCGGAAGTCC | CTCTCAACA |
| | | CCTCAG | AGGAGTT | GCCCCTCAG |
| | | | | GATTAAGTC |
| SPG20 | 23111 | GGCACCTCCTGAA | AGAATGAGACTCTT | TGAAGATCA |
| | | GATCATTCT | GTTTCAACCA | TTCTGCAGA |
| | | | | GAAGTGG |
| SRP19 | 6728 | AGGGAAGTCTTCAT | CAGAAAAACGAGC | CTTCATGCC |
| | | GCCACG | TGCCAGG | ACGTCAGAG |
| | | | | ACTAGAGAT |
| | | | | C |
| SRP72 | 6731 | TGCCACGAGAGCA | GAGGAGTGAGACC | CCACGAGAG |
| | | GAAGATT | TGCGTC | CAGAAGATT |
| | | | | ATGATCT |
| STC2 | 8614 | CCCAGCCATTTCAT | GTAACCTCTATCCG | CATTTCATC |
| | | CACCCT | AGCCGC | ACCCTGCTA |
| | | | | GCAC |
| STOML2 | 30968 | TCAGCTTTAGCCTT | CAAGGAGGGGTGG | CAAGAGAAG |
| | | GGCCTT | GAAAAGG | GGACAGAGC |
| | | | | TTGCTTG |
| SUN2 | 25777 | GGAAGAACCAGGG | GAACCCACACCCT | CTCCAAGAG |
| | | GCTCTTC | GCACTAG | CTTCTGAAA |
| | | | | AGTGG |
| TAPT1 | 202018 | GAGGAACTGTCAA | AGGAAGAAGATGG | GGAGCCTCG |
| | | CGGCCG | CGGCTAC | GCAGCCTCG |
| | | | | GCGGCTCCG |
| | | | | CGC |
| TARS2 | 80222 | GACTCTGAGCTCGA | CCCCTGCTCAAGTG | CTTGTATCA |
| | | AGGACC | AAGAGA | CCCAATCCC |
| | | | | CTTAAAAAG |
| | | | | TAG |
| TBCA | 6902 | AAATCAGAGCGGC | GCCCTCTAGTAAAC | AAATCAGAG |
| | | CAGTGAG | CCGCC | CGGCCAGTG |
| | | | | AG |
| TBKBP1 | 9755 | CTCGGGGCAGGAA | TACACTCTATCAGG | CAGGAAGTT |
| | | GTTTCTG | CGCCCT | TCTGGGTTG |
| | | | | CATCTTAG |
| TCF12 | 6938 | CCGACAATGTGAG | AAAGCATAGCCAG | AGTGGTCTA |
| | | GGTGGAG | AAGTACAGA | ATTGAATTC |
| | | | | AAAACGTAC |
| | | | | TTA |
| THTPA | 79178 | GGCCTTAATGTCAC | CGTGTGGGGTCCTA | CTTAATGTC |
| | | CGAGGT | AGACAC | ACCGAGGTA |
| | | | | GAGAGAAAA |
| | | | | G |
| TIMM10 | 26519 | TTCTCTTCCTGCTT | CCCAGGGGTAGGA | CATCTAAAT |
| | | GGCTCC | GAGTGAA | GCCCAACTC |
| | | | | ATTCTAGTG |
| | | | | AC |
| TIMM10B | 26515 | TTTCGAGGCCAGAC | CTCCTTTCTTCCCC | TTTCGAGGC |
| | | GTTCAG | ATGCCC | CAGACGTTC |
| | | | | AG |
| TIMM29 | 90580 | GGCGGCTCTGAGG | GAGCCCCAGGTTG | CTCTGAGGA |
| | | AGATTTT | ACGTAG | GATTTTGGT |
| | | | | CCCG |
| TIMM8B | 26521 | GTCGCCCAAATCTT | ACCCACGACGACG | AAATCTTCC |
| | | CCCTGT | AAAGAAA | CTGTTTTACA |
| | | | | CCTTTTCTTT |
| | | | | T |
| TIMM9 | 26520 | AGTAACTCAGCAG | TCTGTAGATCATAC | CATCTTCTCT |
| | | CTGCAGG | TGTACCCATTT | AAAATGGTC |
| | | | | TGACTTGGT |
| | | | | AC |
| TLE1 | 7088 | GGGAAAAAGTAAA | TGTACAACCCCAAC | GAACAGAAG |
| | | CCCTGAATGGT | CCGAAG | GATGAGTTT |
| | | | | CACTATTAA |
| | | | | ACT |
| TLE3 | 7090 | CCTGCACCAGGTAT | GAATGGGAAGAGC | TATCAACAG |
| | | CAACAGA | CACTCCC | ATGACTCCA |
| | | | | AATCCTTGG |
| | | | | TAA |
| TM2D3 | 80213 | CAAGCGCTCCATCT | CAGAAGGCTCAAC | CAAGCGCTC |
| | | CCGTG | CGGAAGA | CATCTCCGT |
| | | | | GC |
| TMED5 | 50999 | CGGGCTGGCTTCCT | GTCAACCACGAGG | CTCGCCTCTT |
| | | GAA | AGTCCAG | CACCACCAG |
| | | | | G |
| TOMM70 | 9868 | ACCATGTCCAAGTG | CTCGCTCGCTCATT | GGGACCTTC |
| | | AGCACC | GCTTTC | AGGGTGTCC |
| | | | | GCTGCCCGG |
| | | | | GGC |
| TORIAIP1 | 26092 | TACACAGCAGCGA | TCTAGCCGGGTTCG | GGCGGCGGC |
| | | CGACG | TTTTCC | CCCAGCGAC |
| | | | | TCGCAACTG |
| | | | | CCT |
| TRIM59 | 286827 | TGGTAAGGCAATG | TGGAGGTTAATGCC | TCTAATAGA |
| | | ACCACAAAC | TAGAATGTT | CAGTAAACA |
| | | | | TTTAATGGTT |
| | | | | GC |
| TRMT1 | 55621 | GCAAACTCGGTGA | GGCTCTCTGACCCT | AAACTCGGT |
| | | TCACAGC | CTCTGT | GATCACAGC |
| | | | | ACATC |
| TUBGCP2 | 10844 | TGAAGGAAACAGA | GCGCTTAGCCTGTT | TGAAGGAAA |
| | | CCCTGCG | GTAGTG | CAGACCCTG |
| | | | | CG |
| TUBGCP3 | 10426 | GGACACAAAAGCA | AGGGGACTTTGGCT | GACACAAAA |
| | | AGCCTGG | TCATTT | GCAAGCCTG |
| | | | | GATG |
| TYSND1 | 219743 | AGCAGCTCAGCAG | GGCGCTAGGCAGC | GGGCTGCAG |
| | | GAAGC | TTCA | GGGACGCCC |
| | | | | GCGGGACGG |
| | | | | GGC |
| UBAP2 | 55833 | CATGCCCGGCCTTA | CCCCATTTTCCAAA | ATATTTTTAT |
| | | CTGTAG | GGTTCTCC | ATTTAGAAA |
| | | | | GTAATTATA |
| | | | | AA |
| UBXN8 | 7993 | GGGGACGACTTGC | CGATGCAGTCTGG | GAAACACGG |
| | | CTTTCTT | GAGTTGT | CTACAGACT |
| | | | | ATAACTTTA |
| | | | | AAA |
| UPF1 | 5976 | GCACTGTTACCTCT | CCATGTGCCGCTCA | GCACTGTTA |
| | | CGGTCC | CCT | CCTCTCGGT |
| | | | | CC |
| USP13 | 8975 | CGGAGACTCGCCA | AGGAAGAGAAGAG | GGAGACTCG |
| | | TTGGATT | GTCCCGG | CCATTGGAT |
| | | | | TAAAAATAG |
| USP54 | 159195 | GAAAAGGGGCTAA | TGCTTTTTCGACAT | CCTTTTGTCC |
| | | GCTGGGT | TGGGGTC | TTACTAAAG |
| | | | | ATACTGTCA |
| | | | | AT |
| VPS11 | 55823 | AGATCTAGGACTA | GACCCCTCCGACA | AGATCTAGG |
| | | CCCCGCG | AACAGAT | ACTACCCCG |
| | | | | CG |
| VPS39 | 23339 | ATGTTTTCCCCCTC | CTCTGGCTGGGGA | TTGCAAGAA |
| | | TGGAGT | ATGCTAG | CTAGACTAT |
| | | | | CCCATTTTTA |
| | | | | AT |
| WASHC4 | 23325 | TGGGGGTAGATGG | TCTGCATGGCTTAG | TAGTGGCTT |
| | | GCTAGTG | AGAAAAGGA | TTTCATAAT |
| | | | | ATGTTAGGG |
| | | | | TTT |
| WFS1 | 7466 | CCATGCATCCTTCC | CTCTACAGGAAGG | GTAACCAAG |
| | | CTGGTA | TTCTGGTC | TCCTGACAC |
| | | | | CTTCTATGA |
| | | | | GTC |
| YIF1A | 10897 | CCTCTGTGTGCTCC | TTGGGGTCCCCTCA | CTGTGTGCT |
| | | ATCCC | CTGATC | CCATCCCTG |
| | | | | AG |
| ZC3H18 | 124245 | TGGCCTGTCTTTCT | TCTGAGTCCTGGTC | CTGTCTTTCT |
| | | CTGCAG | TTGGGA | CTGCAGAGT |
| | | | | GGAG |
| ZC3H7A | 29066 | AAAACCCCCAAAT | ACGATGAAAGTGA | CAAATTCAG |
| | | TCAGCCT | CTGAGTACA | CCTATATGC |
| | | | | AATACTGAA |
| | | | | AAA |
| ZDHHC5 | 25921 | TGGCCTTTGACCAA | TTTCCCCGGCCCCT | CTTGCAGAT |
| | | CCTCTG | ACT | TTATAGAGC |
| | | | | AAAATAAAC |
| | | | | TGG |
| ZNF318 | 24149 | TTACAGCCAAGTCC | AGAAGACAAGTCT | GATGGTGTC |
| | | CCTGGA | AGATTGCCTTGA | TCCTTTGTTG |
| | | | | GTGTCTCTT |
| ZNF503 | 84858 | GGTACGGAAGCAG | CCCTCGCTTTCTGC | GTACGGAAG |
| | | TAGCCTC | CCTAAG | CAGTAGCCT |
| | | | | CTTC |
| ABCC1 | 4363 | CCTCTTTCCCTGGG | CCCAGGGTTATGAC | CTCTTTCCCT |
| | | CTTGTT | TGATGCA | GGGCTTGTT |
| | | | | GTCTTTG |
| ATP6AP1 | 537 | ACAGCCAACCAGT | CCCGAGCAAGGAA | CCAACCAGT |
| | | GAGAAGG | CAGTCC | GAGAAGGAG |
| | | | | TGG |
| BRD2 | 6046 | GGGCCAGCAATAA | ATGGCCATGCGAA | AATAAAAGC |
| | | AAGCTCC | CTGATGT | TCCACAGAT |
| | | | | TGTTTGGAT |
| | | | | ATT |
| BRD4 | 23476 | CTGACCAGGAGAC | ACTGATATCTCACG | CTGACCAGG |
| | | ATGCAGG | GGGGCT | AGACATGCA |
| | | | | GG |
| CEP250 | 11190 | ATGTGCTTTGGTCC | GCTAGATGTAGGC | AGTTCAAGA |
| | | CCAGTT | CACTCCC | GGAGGTTGA |
| | | | | AGTGG |
| COMT | 1312 | GTGAAATACCCCTC | CTGGTGGGGAGGA | GTGAAATAC |
| | | CAGCGG | CAAAGTG | CCCTCCAGC |
| | | | | GG |
| CSNK2A2 | 1459 | ACATTTGTGGGCTG | TCCATCTGATTGGC | ATCAAAATA |
| | | AATCAAAA | TAACATTGT | GTGAAGTAC |
| | | | | AAACCCAGA |
| | | | | AAA |
| CSNK2B | 1460 | GGTCAGAAGCCCA | CAGGATGACCCCC | TAAGGCCCA |
| | | GGTTTCT | AATCAGA | AAAGTAGGT |
| | | | | GCTAG |
| CUL2 | 8453 | GAACGTTCCACAC | AGACTCACATCTTT | CTAAATACC |
| | | ACTCCCT | CCCAGTTGT | CACCTTACC |
| | | | | CTGACTATA |
| | | | | GAC |
| DCTPP1 | 79077 | CCGGTATCTTCCCA | AATTGGTCGGAGCT | CGTTCCTAG |
| | | GGGCTA | CTGGAG | TTACCACTC |
| | | | | GGAG |
| DNMT1 | 1786 | TCAAAAGAGAACC | TCATCGCCCCTCCC | CTAGTTTCTA |
| | | CCCACCC | CAT | GCCACCAGG |
| | | | | GAGCTAC |
| EDEM3 | 80267 | GACCCTGTCCACCC | GTCCGTGTTACTCC | GACCCTGTC |
| | | CTCTAG | GCATCC | CACCCCTCT |
| | | | | AG |
| EIF4E2 | 9470 | CCTCACAACACCAC | AGTGATGCAGTTTT | TATAGTGTC |
| | | ACATGA | GAGAGACT | TTCCATGCTT |
| | | | | ATGTTCTTA |
| | | | | AC |
| EIF4H | 7458 | AGAATGGCTGATG | GTGACCACACAAG | TTTTCTGTTG |
| | | CTTCTGC | GTGCATG | GAAGCAAAA |
| | | | | GCTCTTAAA |
| | | | | AT |
| ELOB | 6923 | GAGGTCTAAACAT | AGCAGCCGCGATG | GAGGTCTAA |
| | | CGCCCCC | GTGA | ACATCGCCC |
| | | | | CC |
| ERLEC1 | 27248 | TTGATATGTCGTCT | GGAAGAGGCCGAA | TTGATATGT |
| | | GCCCCG | CCCTTAG | CGTCTGCCC |
| | | | | CG |
| ERO1B | 56605 | GGACCGTCACCATC | AACCGTCCCCTTGG | GACCGTCAC |
| | | TTCCTC | GTC | CATCTTCCTC |
| | | | | TTTT |
| F2RL1 | 2150 | AGCCCCTATAAGC | CCCCATAAATCCAG | CCCTATAAG |
| | | ATTTTGTGT | TTGTTGCC | CATTTTGTGT |
| | | | | AATCCTCTA |
| | | | | AT |
| FKBP10 | 60681 | AAGAGGACAGGAA | AACAAGGAAACAG | AAGAGGACA |
| | | GAGGGGG | GACCCCG | GGAAGAGGG |
| | | | | GG |
| FKBP15 | 23307 | TTGAGGGTACAAG | TCAATTTTGAAGCT | AGTAGACAA |
| | | CACTCCC | AGTTCAGTGGT | GATAATGGC |
| | | | | TTTTCAAGTT |
| | | | | TT |
| FKBP7 | 51661 | AGAGAAACACTGC | CTTTGTGACGCAGG | AGAGAAACA |
| | | CATATAATGTGA | ACAACG | CTGCCATAT |
| | | | | AATGTGATT |
| | | | | TTT |
| FOXRED2 | 80020 | GGCTGAGCAGAGA | CGTGACCCAGATTG | CTGAGCAGA |
| | | GTTCCAG | CAGTGA | GAGTTCCAG |
| | | | | TCG |
| GLA | 2717 | AAAAAGCAGCAGC | AGTCATCGGTGATT | AACTGTTCC |
| | | AGAGTCG | GGTCCG | CGTTGAGAC |
| | | | | TCTC |
| HDAC2 | 3066 | AGGAAAAAGAGGG | CAGCTGGTAAAAG | GAGGGTATA |
| | | TATAGCTCTC | TGTGCGT | GCTCTCATTC |
| | | | | TTATTCATC |
| HYOU1 | 10525 | TCCAGGTTTGACAA | TCCTTCACTCCGGG | ATCACTGCC |
| | | TGGCCA | TATCCA | AGTGTATCT |
| | | | | GAAGGGAAA |
| | | | | AG |
| IMPDH2 | 3615 | TAAACCCCTACTCC | AAGTGCCTTTTTGT | CTTGCTAAT |
| | | CACCCC | GGGGGA | GATCGTTGC |
| | | | | CCTTC |
| LARP1 | 23367 | TGACCATGCTTCCC | GGCACCTAAAGCT | CATCTCAGG |
| | | ACTGAA | CCTCCAG | TGTGAAAAT |
| | | | | GACCTTAGA |
| | | | | ATA |
| LOX | 4015 | CCAGCGGTGACTCC | TCCCTCACGTGATT | GCCGGCCGT |
| | | AGATG | TGAGCC | CCGCGTTCG |
| | | | | CGCCGCGGC |
| | | | | GGT |
| MARK2 | 2011 | TCTTCACATGCCTA | ATCCCACAGCTTTT | CCTGCACCC |
| | | CCAGCC | TGCACC | TCATCCCTTA |
| | | | | TATATTTT |
| MARK3 | 4140 | ACAGCCACGTATG | TGGTATTTACCTCT | ACGTATGCA |
| | | CAAAATATCT | CTGCCTGT | AAATATCTA |
| | | | | ATTTCTTCCT |
| | | | | GA |
| MRPS2 | 51116 | AGGAGCATGCGAG | AGTTTCGACCGCGT | CGGAGGGGC |
| | | GAGGAT | GCAG | GCGGGGACC |
| | | | | CGATGGAGC |
| | | | | GGC |
| MRPS25 | 64432 | CAGGAGTGGGGTT | CGGGTGCTAGCTA | CCTCAGTCT |
| | | CTTGTCC | GTCCTTT | GGACCTCTG |
| | | | | TAAAATG |
| MRPS27 | 23107 | TGGAAAAGTAGCA | TCTGTCACATTGCA | TTATTAATG |
| | | GCTACAGGA | CTCTGT | AACTTATAC |
| | | | | CCAGCTCCA |
| | | | | TTC |
| MRPS5 | 64969 | GCCTTGAACTATAA | ACTCCCTCGTCTTG | TGAAAATAC |
| | | CAATTGCAATC | GTTCTT | TCTTCAGAA |
| | | | | CCTATGTAA |
| | | | | TCG |
| NDUFAF1 | 51103 | TTGCACAGTACCCA | AGTGGCTTCTCCTG | CCTCAGAGC |
| | | CTTCGG | GCAAAG | TCAGAGTTC |
| | | | | CATATAG |
| NDUFAF2 | 91942 | ATGGTGAGCGCCG | GATGCCAGAGTGA | GTTACTAGA |
| | | TTACTAG | AGGGGTC | AGGGCTCCA |
| | | | | GGATG |
| NDUFB9 | 4715 | GGAAAACGCTCCT | AACCCGGGTCTACC | GAAAACGCT |
| | | CTTACCGA | ATAGGA | CCTCTTACC |
| | | | | GATAAACTT |
| | | | | GAA |
| NEK9 | 91754 | GGGAAGAGTGGTG | CATCTGAAGCGAG | GAAGAGTGG |
| | | AAGACCC | CGGGAC | TGAAGACCC |
| | | | | TAAGACATA |
| | | | | TA |
| NGLY1 | 55768 | AGAACTAAGAACA | AGGCATTATTTACC | ATGGGGCAT |
| | | AAATATGGGGCA | TTAGGCTGT | AAATTCAGG |
| | | | | AATAAATCA |
| | | | | TAA |
| NUP210 | 23225 | ATGACATGAGCAG | CTCATCACCTGCTG | ATGACATGA |
| | | TGGTGGC | GCCTG | GCAGTGGTG |
| | | | | GC |
| NUP214 | 8021 | GAAGAATTCCAGG | GGGTTAACCTATGA | ATTTATCTGT |
| | | GATACTTAATCC | AGCTTCCA | ATAACTAGG |
| | | | | TATTGGGGT |
| | | | | GT |
| NUP54 | 53371 | CTCTGAGTAGGACT | TGATCTGACTGGCG | CTCTGAGTA |
| | | CCCCGG | GTTTCC | GGACTCCCC |
| | | | | GG |
| NUP58 | 9818 | CGTACTTTTGCGTG | GGGCGGCTAGATT | GTACTTTTGC |
| | | GTTGCT | AAGTGCT | GTGGTTGCT |
| | | | | CC |
| NUP62 | 23636 | GAAGCACCGATCC | CCAGTCATGCCACT | CGATCCCCA |
| | | CCAAAGA | GAGCTT | AAGAAAATC |
| | | | | CAGTTC |
| NUP88 | 4927 | CAGCCAAGAGGAG | GCGGATTGGCTGTG | CCAAGAGGA |
| | | CAAGGAA | CTCA | GCAAGGAAC |
| | | | | AAAAA |
| NUP98 | 4928 | ACTCTCTTCCTTTC | AGGAATTGACTTA | CAGCCTATT |
| | | CAGCCT | GTGGCTCTGA | AACCTTTTC |
| | | | | AGTACATAT |
| | | | | TGA |
| OS9 | 10956 | GGACCTTGGAGCC | ACTCTTCCCGATTC | CGTTTACAA |
| | | ACGTTTA | CCCGTA | ATAGGAATA |
| | | | | GGGTACGTG |
| PLOD2 | 5352 | GGCAACCTACAGA | AGAAGAGTGGTTA | GGCAACCTA |
| | | ATAGTAATATCTAC | CGGTACAGT | CAGAATAGT |
| | | T | | AATATCTAC |
| | | | | TTT |
| PRKACA | 5566 | GTGCTGCTTTTGAG | TGGCTCCGGCATCC | CTTTTGAGG |
| | | GGATGT | CTA | GATGTTACT |
| | | | | GAGGTTG |
| PTGES2 | 80142 | CTGATCAGCATCCC | CTGAGGGTTCCCTT | CTGATCAGC |
| | | CATCCC | AGCGTC | ATCCCCATC |
| | | | | CC |
| RAE1 | 8480 | ACTCTGCTCATTGC | CAGGACACAAGTA | CTGCTCATT |
| | | GCTCTT | CGGGGAC | GCGCTCTTG |
| | | | | TCTGAAAA |
| RBX1 | 9978 | TGCGACAGCCCCTT | CGTCACGCCGATCA | CCCTTTAAG |
| | | TAAGAG | ACTCTA | AGGCGTGGT |
| | | | | CAC |
| RIPK1 | 8737 | AGTCTTGCCCTGAG | ATCCGAAGAGCCA | CCCTGAGGT |
| | | GTTTTCT | TCGTCAC | TTTCTCTCTG |
| | | | | TTTTCTTTA |
| SDF2 | 6388 | TGGTGTTGCGATTA | TTCGCCATTAGCTT | CGATTAAGA |
| | | AGATGCC | CCGGTT | TGCCTTAGA |
| | | | | ACAATTCAG |
| | | | | TTC |
| SIGMAR1 | 10280 | ATCCGAGATCTCAG | GGAGCCTAGGGTT | CAATCGCAC |
| | | CCCAGT | CCGAAG | ATGACACTA |
| | | | | TCAGGGTAT |
| | | | | TC |
| SIL1 | 64374 | CTTGGAACTGATGC | GAGCAAGTGACGA | GTTGTTGGG |
| | | CCACCA | CATGGGA | AGGATTAAA |
| | | | | TGAGAATAC |
| | | | | ATA |
| TBK1 | 29110 | TGAGACATGCACA | CACCCTTGGAAGC | TGCACACAT |
| | | CATACACGT | GAGTACC | ACACGTAAA |
| | | | | TATCTACATT |
| | | | | AT |
| TMEM97 | 27346 | TGTCCACGAGCCTC | AAAGTTGGGTTAG | TCCACGAGC |
| | | CTC | GAGCGGG | CTCCTCTTCT |
| | | | | C |
| TOR1A | 1861 | ATCCTCAATCCCCT | GCCCTGAAGAAAG | ATCCTCAAT |
| | | AGCCCC | ATGGCCT | CCCCTAGCC |
| | | | | CC |
| UGGT2 | 55757 | GGAAGGAGGTGGT | AGTAACGGACTCG | GAAGGAGGT |
| | | GATGCTC | AGCTCCT | GGTGATGCT |
| | | | | CAG |
| ZYG11B | 79699 | AAGTGTGATGGAA | GCAACTTCAGCCA | GATGGAAAT |
| | | ATTTTGGCT | GGTCTTC | TTTGGCTATT |
| | | | | CTTTAACTGT |
| | | | | T |
| ACE2 | 59272 | CTGGGACTCCAAA | CGCCCAACCCAAG | CAAAATCAG |
| | | ATCAGGGA | TTCAAAG | GGATATGGA |
| | | | | GGCAAACAT |
| | | | | C |

Identification of Essential Genes for siRNA and Cas9 Knockout Screen

Here, longitudinal imaging in A549 cells was used to assess cell viability (FIG. 3A-F). For benchmarking, relative cell viability was measured by CellTiter-Glo Luminescent Cell Viability Assay (Promega; G7571) as per manufacturer's instructions. Briefly, two passages post-nucleofection A549 siRNA pools cultured in 96-well tissue-culture treated plates (Corning, #3595) were lysed in the CellTIter-Glo reagent, by removing spent media and adding 100 μl of the CellTiter-Glo reagent containing the CellTiter-Glo buffer and CellTiter-Glo Substrate. Cells were placed on an orbital shaker for 2 minutes on a SpectraMax iD5 (Molecular Devices) and then incubated in the dark at room temperature for 10 minutes. Completely lysed cells were pipette mixed and 25 μl were transferred to a 384-well assay plate (Corning, #3542). The luminescence was recorded on a SpectraMax iD5 (Molecular Devices) with an integration time of 0.25 seconds per well. Luminescence readings were all normalized to the without-sgRNA control condition.

To determine cell viability in Caco-2 knockouts we used longitudinal imaging (FIG. 3A-F). All gene knockout pools were maintained for a minimum of six passages to determine the effect of loss of protein function on cell fitness prior to viral infection. Viability was determined through longitudinal imaging and automated image analysis using a Celigo Imaging Cytometer (Celigo). Each gene knockout pool was split in triplicate wells on separate plates. Every day, except the day of seeding, each well was scanned and analyzed using built in ‘Confluence’ imaging parameters using auto-exposure and autofocus with an offset of −45 μm. Analysis was performed with standard settings except for an intensity threshold setting of 8. Confluency was averaged across 3 wells and plotted over time. Viability genes were determined as pools that were less than 20% confluent 5 days post seeding following 6 passages.

Genes deemed essential were excluded from the knockout screen.

Cells, Virus, and Infections for Caco-2 Cas9 Knockout Screen

Wild-type and CRISPR edited Caco-2 cells were grown at 37° C., 5% CO₂ in DMEM, 10% FBS. SARS-CoV-2 stocks were grown and titered on Vero E6 cells as described previously (A. S. Jureka, et al., Propagation, Inactivation, and Safety Testing of SARS-CoV-2. Viruses. 12 (2020), doi:10.3390/v12060622). Wild-type and CRISPR edited Caco-2 cell lines were infected with SARS-CoV-2 at an MOI of 0.01 in DMEM supplemented with 2% FBS. 72 hours post-infection, supernatants were harvested and stored at −80° C. and the Caco-2 WT/CRISPR KO cells were fixed with 10% neutral buffered formalin (NBF) for 1 hour at room temperature to enable further analysis.

Focus Forming Assay for Caco-2 Cas9 Knockout Screen

Vero E6 cells were plated into 96 well plates at confluence (50,000 cells/well) in DMEM supplemented with 10% heat-inactivated FBS (Gibco). Prior to infection, supernatants from infected Caco-2 WT/CRISPR KO cells were thawed and serially diluted from 10⁻¹ to 10⁻⁸. Growth media was removed from the Vero E6 cells and 40 μl of each virus dilution was plated. After 1 hour adsorption at 37° C., 5% CO₂, 40 μl of 2.4% microcrystalline cellulose (MCC) overlay supplemented with DMEM powdered media (Gibco) to a concentration of 1×was added to each well of the 96 well plate to achieve a final MCC overlay concentration of 1.2%. Plates were then incubated at 37° C., 5% CO₂ for 24 hours. The MCC overlay was gently removed and cells were fixed with 10% NBF for 1 hour at room-temperature. After removal of NBF, monolayers were washed with ultrapure water and ice-cold 100% methanol/0.3% H₂O₂ was added for 30 minutes to permeabilize the cells and quench endogenous peroxidase activity. Monolayers were then blocked for 1 hour in PBS with 5% non-fat dry milk (NFDM). After blocking, monolayers were incubated with SARS-CoV N primary antibody (Novus Biologicals; NB100-56576-1:2000) for 1 hour at room temperature in PBS, 5% NFDM. Monolayers were washed with PBS and incubated with an HRP-Conjugated secondary antibody for 1 hour at room temperature in PBS with 5% NFDM. Secondary was removed, monolayers were washed with PBS, and then developed using TrueBlue substrate (KPL) for 30 minutes. Plates were imaged on a Bio-Rad Chemidoc utilizing a phosphorscreen and foci were counted by eye to calculate focus forming units per ml (FFU/ml) for each knockout. The original formalin-fixed Caco-2 WT/CRISPR KO cells were stained with Dapi (Thermo Scientific) and imaged on a Cytation 5 plate reader to determine cell viability. Wells containing no cells were excluded from further analyses.

Quantitative Analysis and Scoring of Knockdown and Knockout Library Screens

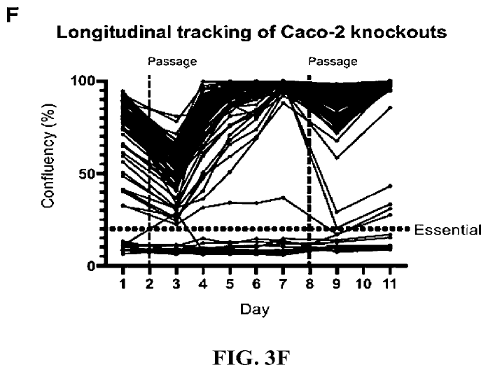

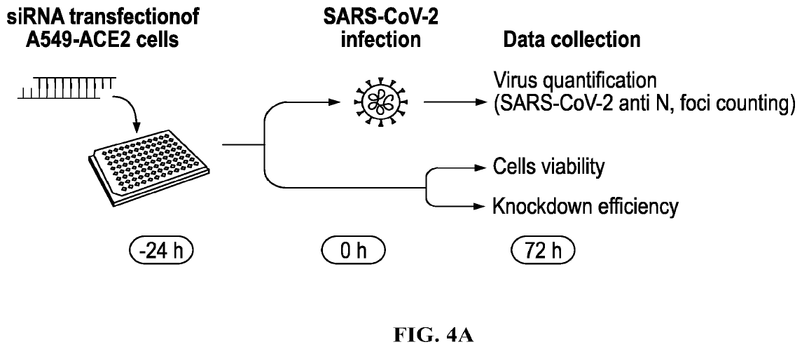

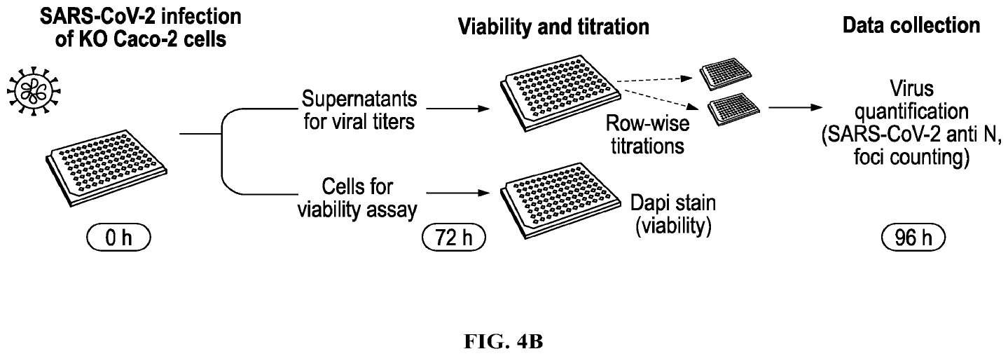

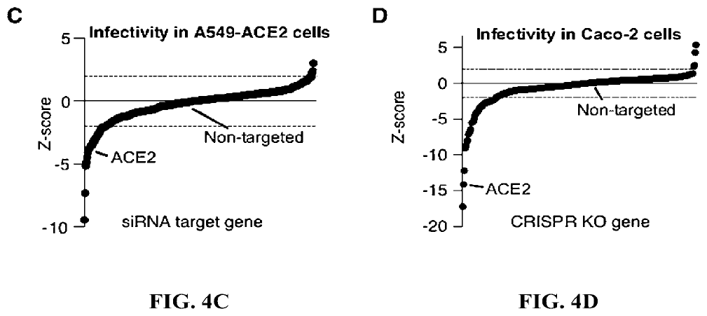

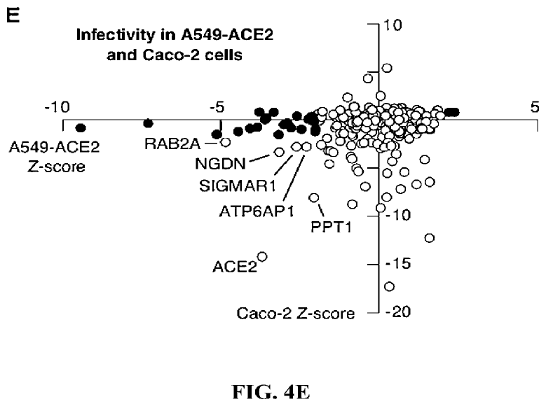

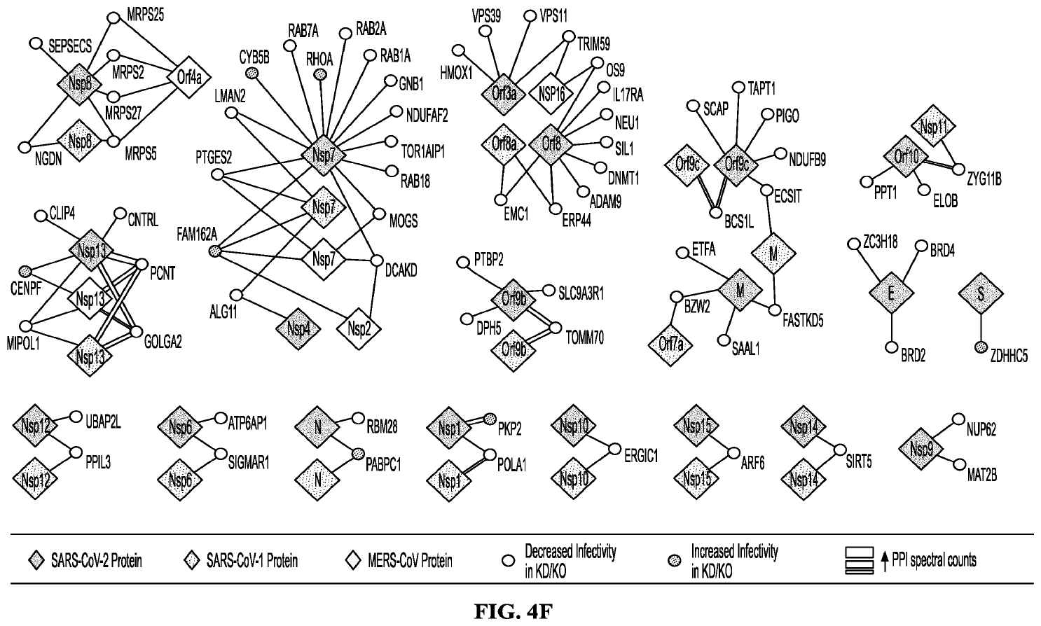

Virus readout by qPCR (A549-ACE2, expressed as PFU/ml) and focus forming assay readouts (Caco-2, FFU/ml) were processed using the RNAither package (https://www.bioconductor.org/packages/release/bioc/html/RNAither.html) in the statistical computing environment R. The two datasets were normalized separately, using the following method. The readouts were first log transformed (natural logarithm), and robust Z-scores (using median and MAD “median absolute deviation” instead of mean and standard deviation) were then calculated for each 96-well plate separately. Z-scores of multiple replicates of the same perturbation were averaged into a final Z-score for presentation in FIG. 4A-F. No filtering was done based on differences in replicate Z-scores. It is suggested to consult the replicate Z-scores for all genes/perturbations of interest. The A549-ACE2 siRNA screen includes 3 replicates (or more) of each perturbation, and the Caco-2 CRISPR screen includes 2 replicates (or more) of each perturbation. The results from the A549-ACE2 screen cover all 332 screened genes (331 SARS-CoV-2 interactors plus ACE2). The results from the Caco-2 screen cover 286 of the screened genes plus ACE2. The remaining Caco-2 genes were either deemed essential, failed editing, or failed in the focus forming assay.

Referring to FIG. 4A, A549-ACE2 cells were transfected with siRNA pools targeting each of the human genes from the SARS-CoV-2 interactome, followed by infection with SARS-CoV-2 and virus quantification using RT-qPCR. Cell viability and knockdown efficiency in uninfected cells was determined in parallel.

Referring to FIG. 4B, Caco-2 cells with CRISPR knockouts of each human gene from the SARS-CoV-2 interactome were infected with SARS-CoV-2, and supernatants were serially diluted and plated onto Vero E6 cells for quantification. Viabilities of the uninfected CRISPR knockout cells were determined in parallel.

Referring to FIG. 4C and FIG. 4D, a plot of results from the infectivity screens in A549-ACE2 knockdown cells (FIG. 4C) and Caco-2 knockout cells (FIG. 4D) sorted by Z-score (Z<0, decreased infectivity; Z>0 increased infectivity) is shown. Negative controls (non-targeting control for siRNA, nontargeted cells for CRISPR) and positive controls (ACE2 knockdown/knockout) are highlighted.

Referring to FIG. 4E, results from both assays with potential hits (14>2) highlighted in red (A549-ACE2), yellow (Caco-2) and orange (both) are shown.

Referring to FIG. 4F, pan-coronavirus interactome reduced to human preys with significant increase (red nodes) or decrease (blue nodes) in SARS-CoV2 replication upon knockdown/knockout is shown. Viral proteins baits from SARS-CoV-2 (red), SARS-CoV-1 (orange) and MERS-CoV (yellow) are represented as diamonds. The thickness of the edge indicates the strength of the PPI in spectral counts. KD=Knockdown; KO=Knockout; PPI=protein-protein interaction.

See also Tables 5 Å-G provide in U.S. Provisional Application No. 63/091,929 filed on Oct. 15, 2020, expressly incorporated by reference herein.

Antiviral Drug and Cytotoxicity Assays (A549-ACE2 Cells)

2,500 A549-ACE2 cells were seeded into 96- or 384-well plates in DMEM (10% FBS) and incubated for 24 hours at 37° C., 5% CO₂. Two hours prior to infection, the media was replaced with 120 μl (96 well format) or 50 μl (384 well format) of DMEM (2% FBS) containing the compound of interest at the indicated concentration. At the time of infection, the media was replaced with virus inoculum (MOI 0.1 PFU/cell) and incubated for 1 hour at 37° C., 5% CO₂. Following the adsorption period, the inoculum was removed, replaced with 120 μl (96 well format) or 50 μl (384 well format) of drug-containing media, and cells incubated for an additional 72 hours at 37° C., 5% CO₂. At this point, the cell culture supernatant was harvested, and viral load assessed by RT-qPCR (as described in ‘Viral infection and quantification assay in A549-ACE2 cells’). Viability was assayed using the CellTiter-Glo assay following the manufacturer's protocol (Promega). Luminescence was measured in a Tecan Infinity 2000 plate reader, and percentage viability calculated relative to untreated cells (100% viability) and cells lysed with 20% ethanol or 4% formalin (0% viability), included in each experiment.

Antiviral Drug and Cytotoxicity Assays (Vero E6 Cells)

Viral growth and cytotoxicity assays in the presence of inhibitors were performed as previously described (Gordon, et al. A SARS-CoV-2 protein interaction map reveals targets for drug repurposing. Nature (2020). 2,000 Vero E6 cells were seeded into 96-well plates in DMEM (10% FBS) and incubated for 24 hours at 37° C., 5% CO₂. Two hours before infection, the medium was replaced with 100 μl of DMEM (2% FBS) containing the compound of interest at concentrations 50% greater than those indicated, including a DMSO control. SARS-CoV-2 virus (100 PFU; MOI 0.025) was added in 50 μl of DMEM (2% FBS), bringing the final compound concentration to those indicated. Plates were then incubated for 48 hours at 37° C. After infection, supernatants were removed and cells were fixed with 4% formaldehyde for 24 hours prior to being removed from the BSL3 facility. The cells were then immunostained for the viral NP protein (rabbit anti-sera produced in the Garcia-Sastre lab; 1:10,000) with a DAPI counterstain. Infected cells (488 nm) and total cells (DAPI) were quantified using a Celigo (Nexcelcom) imaging cytometer. Infectivity is measured by the accumulation of viral NP protein in the nucleus of the cells (fluorescence accumulation). Percent infection was quantified as (Infected cells/Total cells)−Background)*100 and the DMSO control was then set to 100% infection for analysis. The IC₅₀ and IC₉₀ for each experiment was determined using the Prism (GraphPad Software) software. Cytotoxicity measurements were performed using the MTT assay (Roche), according to the manufacturer's instructions. Cytotoxicity was performed in uninfected Vero E6 cells with same compound dilutions and concurrent with viral replication assay. All assays were performed in biologically independent triplicates.

Co-Immunoprecipitation Assays for Orf9b and Tom70

HEK293T and A549 cells were transfected with the indicated mammalian expression plasmids using Lipofectamine 2000 (Invitrogen) and TranslT-X2 (Minis Bio) respectively. 24 hours post-transfection, cells were harvested and lysed in NP-40 lysis buffer (0.5% Nonidet P 40 Substitute (NP-40; Fluka Analytical), 50 mM Tris-HCl, pH 7.4 at 4° C., 150 mM NaCl, 1 mM EDTA) supplemented with cOmplete mini EDTA-free protease and PhosSTOP phosphatase inhibitor cocktails (Roche). Clarified cell lysates were incubated with Streptactin Sepharose beads (IBA) for 2 hours at 4° C., followed by five washes with NP-40 lysis buffer. Protein complexes were eluted in the SDS loading buffer and were analyzed by western blotting with the indicated antibodies.

Quantification of Tom70 Downregulation in HeLaM Cells Overexpressing Orf9b

HeLaM cells were transiently transfected with plasmids encoding GFP-Strep, SARS-CoV-1 Orf9b-Strep or SARS-CoV-2 Orf9b-Strep. The next day, the cells were fixed using 4% paraformaldehyde and immunostained with antibodies against Strep tag, and Tom20 or Tom70. Representative images for each construct were captured by acquiring a single optical section using a Nikon A1 confocal fitted with a CFI Plan Apochromat VC 60×oil objective (NA 1.4). For image quantification multiple fields of view were captured for each construct using a CFI Super Plan Fluor ELWD 40×objective (NA 0.6). The mean fluorescent intensity for Tom20 and Tom70 was measured by manually drawing a region of interest around each cell using ImageJ. Between 30 and 60 cells were quantified for each construct.

Quantification of Tom70 Downregulation in Infected Caco-2 Cells

Caco-2 cells were seeded on glass coverslips in triplicate and infected with SARS-CoV-2 at an MOI of 0.1 as described above. At 24 hours post-infection, cells were fixed with 4% paraformaldehyde and immunostained with antibodies against Tom70, Tom20 and Orf9b. For signal quantification images of non-infected and neighbouring infected cells were acquired using a LSM800 confocal laser-scanning microscope (Zeiss) equipped with a 63×, 1.4 NA oil objective and the Zen blue software (Zeiss). The mean fluorescence intensity of each cell was measured by ImageJ software. 43 cells were quantified for each condition, infected or non-infected, from three independent experiments.

Co-Expression and Purification of Orf9b-Tom70 (109-End) Complexes

SARS-CoV-2 Orf9b and Tom70 (residues 109-end) were coexpressed using a pET29-b(+) vector backbone where Orf9b was tag-less and Tom70 had an N-terminal 10×His-tag and SUMO-tag. LOBSTR E. coli cells transformed with the above construct were grown at 37° C. till O.D. (600 nm)=0.8 and the expression was induced at 37° C. with 1 mM IPTG for 4 hours. Frozen cell pellets were resuspended in 25 ml lysis buffer (200 mM NaCl, 50 mM Tris-HCl pH 8.0, 10% v/v glycerol, 2 mM MgCl₂) per liter cell culture, supplemented with cOmplete protease inhibitor tablets (Roche), 1 mM PMSF (Sigma), 100 μg/ml lysozyme (Sigma), 5 μg/ml DNaseI (Sigma), and then homogenized with an immersion blender (Cuisinart). Cells were lysed by 3×passage through an Emulsiflex C3 cell disruptor (Avestin) at ˜15,000 psi, and the lysate clarified by ultracentrifugation at 100,000×g for 30 minutes at 4° C. The supernatant was collected, supplemented with 20 mM imidazole, loaded into a gravity flow column containing Ni-NTA superflow resin (Qiagen), and rocked with the resin at 4° C. for 1 hour. After allowing the column to drain, resin was rinsed twice with 5 column volumes (cv) of wash buffer (150 mM KCl, 30 mM Tris-HCl pH 8.0, 10% v/v glycerol, 20 mM imidazole, 0.5 mM tris(hydroxypropyl)phosphine (THP, VWR)) supplemented with 2 mM ATP (Sigma) and 4 mM MgCl₂, then washed with 5 cv wash buffer with 40 mM imidazole. Resin was then rinsed with 5 cv Buffer A (50 mM KCl, 30 mM Tris-HCl pH 8.0, 5% glycerol, 0.5 mM THP) and protein was eluted with 2×2.5 cv Buffer A+300 mM imidazole. Elution fractions were combined, supplemented with Ulp1 protease, and rocked at 4° C. for 2 hours. Ulp1-digested Ni-NTA eluate was diluted 1:1 with additional Buffer A, loaded into a 50 ml Superloop, and applied to a MonoQ 10/100 column on an Äkta pure system (GE Healthcare) using 100% Buffer A, 0% Buffer B (1000 mM KCl, 30 mM Tris-HCl pH 8.0, 5% glycerol, 0.5 mM THP). The MonoQ column was washed with 0%-40% Buffer B gradient over 15 cv, peak fractions were analyzed by SDS-PAGE and the identity of tagless Tom70(109-end) and Orf9b proteins confirmed by intact protein mass spectrometry (Xevo G2-XS Mass Spectrometer, Waters). Peak fractions eluting at −15% B contained relatively pure Tom70(109-end) and Orf9b, and these were concentrated using 10 kDa Amicon centrifugal filter (Millipore) and further purified by size exclusion chromatography using a Superdex 200 increase 10/300 GL column (GE healthcare) in buffer containing 150 mM KCl, 20 mM HEPES-NaOH pH 7.5, 0.5 mM THP. The sole size-exclusion peak contained both Tom70(109-end) and Orf9b, and the center fraction was used directly for cryo-EM grid preparation.

Expression and Purification of SARS-CoV-2 Orf9b

Orf9b with N-terminal 10×His-tag and SUMO-tag was expressed using a pET-29b(+) vector backbone. LOBSTR E. coli cells transformed with the above construct were grown at 37° C. until reaching O.D. (600 nm)=0.8 and the expression was induced at 37° C. with 1 mM IPTG for 6 hours. Frozen cell pellets were lysed, homogenized, clarified, and subject to Ni affinity purification as described above for Orf9b-Tom70 complexes, with several small changes. Lysis buffers and Ni-NTA wash buffers contained 500 mM NaCl, and an additional wash step using 10 cv wash buffer+0.2% TWEEN20+500 mM NaCl was carried out prior to the ATP wash. Orf9b was eluted from Ni-NTA resin in Buffer A (50 mM NaCl, 25 mM Tris pH 8.5, 5% glycerol, 0.5 mM THP) supplemented with 300 mM imidazole. This eluate was diluted 1:1 with additional Buffer A, loaded into a 50 ml Superloop, and applied to a MonoQ 10/100 column on an Akta pure system (GE Healthcare) using 100% Buffer A, 0% Buffer B (1000 mM NaCl, 25 mM Tris-HCl pH 8.5, 5% glycerol, 0.5 mM THP). The MonoQ column was washed with 0%-40% Buffer B gradient over 15 cv, and relatively pure Orf9b eluted at 20-25% Buffer B, whereas Orf9b and contaminating proteins eluted at 30-35% buffer B. Fractions from these two peaks were combined and incubated with Ulp1 and HRV3C proteases at 4° C. for 2 hours, supplemented with 10 mM imidazole, then thrice flowed back through 1 ml of Ni-NTA resin equilibrated with size-exclusion buffer (as above)+10 mM imidazole. The reverse-Ni purified sample was concentrated using 10 kDa Amicon centrifugal filter and then further purified by size exclusion chromatography using a Superdex 200 increase 10/300 GL column.

Expression and Purification of Tom70(109-End)

Tom70 (109-end) with N-terminal 10×His-tag and SUMO-tag and C-terminus Spy-tag, HRV-3C protease cleavage site, and eGFP-tag was expressed using a pET-21(+) vector backbone. LOB STR E. coli cells transformed with the above construct were grown at 37° C. till O.D. (600 nm)=0.8 and the expression was induced at 16° C. with 0.5 mM IPTG overnight. The soluble domain of Tom70 (Tom70 (109-end)) was purified as described in (A. C. Y. Fan, et al., Hsp90 functions in the targeting and outer membrane translocation steps of Tom70-mediated mitochondrial import. J. Biol. Chem. 281, 33313-33324 (2006)) with some modifications. Frozen cell pellets of LOB STR E. coli transformed with the above construct were resuspended in 50 ml lysis buffer (500 mM NaCl, 20 mM KH₂PO₄ pH 7.5) per liter cell culture, supplemented with 1 mM PMSF (Sigma) and 100 ug/ml, and homogenized. Cells were lysed by 3× passage through an Emulsiflex C3 cell disruptor (Avestin) at ˜15,000 psi, and the lysate clarified by ultracentrifugation at 100,000×g for 30 minutes at 4° C. The supernatant was collected, supplemented with 20 mM imidazole, loaded into a gravity flow column containing Ni-NTA superflow resin (Qiagen), and rocked with the resin at 4° C. for 1 hour. After allowing the column to drain, resin was rinsed with twice with 5 column volumes (cv) of wash buffer (500 mM KCl, 20 mM KH₂PO₄ pH 8.0, 20 mM imidazole, 0.5 mM THP) supplemented with 2 mM ATP-4 mM MgCl₂, then washed with 5 cv wash buffer with 40 mM imidazole. Bound Tom70 (109-end) was then cleaved from the resin by 2 hour incubation with Ulp1 protease in 4 cv elution buffer (150 mM KCl, 20 mM KH₂PO₄ pH 8.0, 5 mM imidazole, 0.5 mM THP). After cleavage with Ulp1, the flow through was collected along with a 2 cv rinse of the resin with additional elution buffer. These fractions were combined and HRV3C protease was added to remove the C-terminal EGFP tag (1:20 HRV3C to Tom70). After 2 hour HRV3C digestion at 4° C., the double-digested Tom70(109-end) was concentrated using a 30 kDa Amicon centrifugal filter (Millipore) and further purified by size exclusion chromatography using a Superdex 200 increase 10/300 GL column (GE healthcare) in buffer containing 150 mM KCl, 20 mM HEPES-NaOH pH 7.5, 0.5 mM THP.

Prediction of SARS-CoV-2 Orf9b Internal Mitochondrial Targeting Sequence

Orf9b was analyzed for the presence of an internal mitochondrial targeting sequence (i-MTS) as described in (S. Backes, et al., Tom70 enhances mitochondrial preprotein import efficiency by binding to internal targeting sequences. J. Cell Biol. 217, 1369-1382 (2018)) using the TargetP-2.0 server (J. J. Almagro Armenteros, et al., Detecting sequence signals in targeting peptides using deep learning. Life Sci Alliance. 2 (2019), doi:10.26508/lsa.201900429). Sequences corresponding to Orf9b N-terminal truncations of 0 to 62 residues were submitted to the TargetP-2.0 server, and the probability of the peptides containing an MTS plotted against the numbers of residues truncated. A similar analysis using the MitoFates server (Y. Fukasawa, et al., MitoFates: improved prediction of mitochondrial targeting sequences and their cleavage sites. Mol. Cell. Proteomics. 14, 1113-1126 (2015)) predicted that Orf9b residues 54-63 were the most likely to comprise a presequence MTS based on propensity to form a positively charged amphipathic helix. Notably this analysis was consistent with the secondary structure prediction from JPRED (A. Drozdetskiy, et al., JPred4: a protein secondary structure prediction server. Nucleic Acids Res. 43, W389-94 (2015)).

CryoEM Sample Preparation and Data Collection

3 μL of Orf9b-Tom70 complex (12.5 μM) was added to a 400 mesh 1.2/1.3R Au Quantifoil grid previously glow discharged at 15 mA for 30 seconds. Blotting was performed with a blot force of 0 for 5 seconds at 4° C. and 100% humidity in a FEI Vitrobot Mark IV (ThermoFisher) prior to plunge freezing into liquid ethane. 1534 118-frame super-resolution movies were collected with a 3×3 image shift collection strategy at a nominal magnification of 105,000× (physical pixel size: 0.834 Å/pix) on a Titan Krios (ThermoFisher) equipped with a K3 camera and a Bioquantum energy filter (Gatan) set to a slit width of 20 eV. Collection dose rate was 8 e-/pixel/second for a total dose of 66 e-/A2. Defocus range was 0.7 um to 2.4 um. Each collection was performed with semi-automated scripts in SerialEM (D. N. Mastronarde, Automated electron microscope tomography using robust prediction of specimen movements. J Struct. Biol. 152, 36-51 (2005)).

CryoEM Image Processing and Model Building

1534 movies were motion corrected using Motioncor2 (S. Q. Zheng, et al., MotionCor2: anisotropic correction of beam-induced motion for improved cryo-electron microscopy. Nat. Methods. 14, 331-332 (2017)) and dose-weighted summed micrographs were imported in cryosparc (v2.15.0). 1427 micrographs were curated based on CTF fit (better than 5 Å) from a patch CTF job. Template-based particle picking resulted in 2,805,121 particles and 1,616,691 particles were selected after 2D-classification. Five rounds of 3D-classification using multi-class ab-initio reconstruction and heterogenous refinement yielded 178,373 particles. Homogenous refinement of these final particles led to a 3.1 Å electron density map which was used for model building. The reconstruction was filtered by the masked FSC and sharpened with a b-factor of −145.

To build the model of Tom70(109-end), the crystal structure of Saccharomyces cerevisiae Tom71 (PDB ID: 3fp3; sequence identity 25.7%) was first fit into the cryoEM density as a rigid body in UCSF ChimeraX and then relaxed into the final density using Rosetta FastRelax mover in torsion space. This model, along with a BLAST alignment of the two sequences (S. F. Altschul, et al., Gapped BLAST and PSI-BLAST: a new generation of protein database search programs. Nucleic Acids Res. 25, 3389-3402 (1997)), was used as a starting point for manual building using COOT (P. Emsley, K. Cowtan, Coot: model-building tools for molecular graphics. Acta Crystallogr. D Biol. Crystallogr. 60, 2126-2132 (2004)). After initial building by hand the regions with poor density fit/geometry were iteratively rebuilt using Rosetta (R. Y.-R. Wang, et al., Automated structure refinement of macromolecular assemblies from cryo-EM maps using Rosetta. Elife. 5 (2016), doi:10.7554/eLife.17219). Orf9b was built de novo into the final density using COOT, informed and facilitated by the predictions of the TargetP-2.0, MitoFates, and JPRED servers. The Orf9b-Tom70 complex model was submitted to the Namdinator web server (R. T. Kidmose, et al., Namdinator—automatic molecular dynamics flexible fitting of structural models into cryo-EM and crystallography experimental maps. IUCrJ. 6, 526-531 (2019)) and further refined in ISOLDE 1.0 (T. I. Croll, ISOLDE: a physically realistic environment for model building into low-resolution electron-density maps. Acta Crystallogr D Struct Biol. 74, 519-530 (2018)) using the plugin for UCSF ChimeraX (T. D. Goddard, et al., UCSF ChimeraX: Meeting modern challenges in visualization and analysis. Protein Sci. 27, 14-25 (2018)). Final model B-factors were estimated using Rosetta. The model was validated using phenix.validation_cryoem (P. V. Afonine, et al., New tools for the analysis and validation of cryo-EM maps and atomic models. Acta Crystallogr D Struct Biol. 74, 814-840 (2018)). The final model contains residues 109-272, 298-600 of human Tom70, and 39-76 of SARS-CoV-2 Orf9b. Molecular interface between Orf9b and Tom70 was analyzed using the PISA web server (E. Krissinel, K. Henrick, Inference of macromolecular assemblies from crystalline state. J. Mol. Biol. 372, 774-797 (2007)). Figures were prepared using UCSF ChimeraX.

Computational Human Genetics Analysis

To look for genetic variants associated with the list of proteins that had a significant impact on SARS-CoV-2 replication, the largest proteomic GWAS study to date was used (B. B. Sun, et al., Genomic atlas of the human plasma proteome. Nature. 558, 73-79 (2018)). IL17RA was identified as one of the proteins assayed in Sun et al.'s proteomic GWAS. It was observed that IL17RA had multiple cis-acting protein quantitative trait loci (pQTLs) at a corrected p-value 1×10⁻⁵, where cis-acting is defined as within 1 MB of the transcription start site of IL17RA.

The GSMR method (Z. Zhu, et al., Causal associations between risk factors and common diseases inferred from GWAS summary data. Nat. Commun. 9, 224 (2018)) was used to perform MR using near-independent (linkage disequilibrium or LD r²=0.05) cis-pQTLs for IL17RA. The advantage of GSMR method over conventional MR methods is two-fold; first, GSMR performs MR adjusting for any residual correlation between selected genetic variants by default. Second, GSMR has a built-in method called HEIDI (heterogeneity in dependent instruments)-outlier that performs heterogeneity tests in the near-independent genetic instruments and remove potentially pleiotropic instruments (i.e., where there is evidence of heterogeneity at p<0.01). Details of the GSMR and HEIDI method have been published previously (Z. Zhu, et al., Causal associations between risk factors and common diseases inferred from GWAS summary data. Nat. Commun. 9, 224 (2018)).

Summary statistics generated by COVID-19 Human Genetics Initiative (COVID-HGI) (round 3; https://www.covidl9hg.org/results/) for COVID-19 vs. population, hospitalized COVID-19 vs. population and hospitalized COVID-19 vs. non-hospitalized COVID-19 were used for IL17RA MR analysis. Te 1000 genomes phase 3 European population genotype data was used to derive the LD correlation matrix for this analysis. The phenotype definitions as provided by COVID-HGI are as follows. COVID-19 vs. population: Case, individuals with laboratory confirmation of SARS-CoV-2 infection, EHR/ICD coding/Physician-confirmed COVID-19, or self-reported COVID-19 positive; control, everybody that is not a case. Hospitalized COVID-19 vs. population: case, hospitalized, laboratory confirmed SARS-CoV-2 infection or hospitalization due to COVID-19-related symptoms; control, everybody that is not a case, e.g., population. Hospitalized COVID-19 vs. non-hospitalized COVID-19: case, hospitalized, laboratory confirmed SARS-CoV-2 infection or hospitalization due to COVID-19-related symptoms; control, laboratory confirmed SARS-CoV-2 infection and not hospitalized 21 days after the test.

Infections and Treatments for IL17A Treatment Studies

The WA-1 strain (BEI resources) of SARS-CoV-2 was used for all experiments. All live virus experiments were performed in a BSL3 lab. SARS-CoV-2 stocks were passaged in Vero E6 cells (ATCC) and titer was determined via plaque assay on Vero E6 cells as previously described (A. N. Honko, et al., Rapid Quantification and Neutralization Assays for Novel Coronavirus SARS-CoV-2 Using Avicel RC-591 Semi-Solid Overlay, doi:10.20944/preprints202005.0264.v1). Briefly, virus was diluted 1:10²-1:10⁶ and incubated for 1 hour on Vero E6 cells before an overlay of Avicel and complete DMEM (Sigma Aldrich, SLM-241) was added. After incubation at 37° C. for 72 hours, the overlay was removed and cells were fixed with 10% formalin, stained with crystal violet, and counted for plaque formation. SARS-CoV-2 infections of A549-ACE2 cells were done at a MOT of 0.05 for 24 hours. Inhibitors and cytokines were added concurrently with virus. All infections were done in technical triplicate. Cells were treated with the following compounds: Remdesivir (SELLECK CHEMICALS LLC, 58932) and IL-17A (Millipore-Sigma, SRP0675).

RNA Extraction, RT, and Quantitative RT-PCR for IL17 Å Treatment Studies

Total RNA from samples was extracted using the Direct-zol RNA kit (Zymogen, R2060) and quantified using the NanoDrop 2000c (ThermoFisher). cDNA was generated using 500 ng for infected A549-ACE2 cells with Superscript III reverse transcription (ThermoFisher, 18080-044) and oligo(dT)₁₂₋₁₈ (ThermoFisher, 18418-012) and random hexamer primers (ThermoFisher, S0142). Quantitative RT-PCR reactions were performed on a CFX384 (BioRad) and delta cycle threshold (ACt) was determined relative to RPL13 Å levels. Viral detection levels and target host genes in treated samples were normalized to water-treated controls. The SYBR green qPCR reactions contained 5 μl of 2× Maxima SYBR green/Rox qPCR Master Mix (ThermoFisher; K0221), 2 μl of diluted cDNA, and 1 nmol of both forward and reverse primers, in a total volume of 10 μl. The reactions were run as follows: 50° C. for 2 minutes and 95° C. for 10 minutes, followed by 40 cycles of 95° C. for 5 seconds and 62° C. for 30 seconds. Primer efficiencies were around 100%. Dissociation curve analysis after the end of the PCR confirmed the presence of a single and specific product. qRT-PCR primers were used against the SARS-CoV-2 E gene

(PF_042_nCoV_E_F:
ACAGGTACGTTAATAGTTAATAGCGT;
PF_042_nCOV_E_R:
ATATTGCAGCAGTACGCACACA),
the CXCL8 gene (CXCL8 For:
ACTGAGAGTGATTGAGAGTGGAC;
CXCL8 Rev:
AACCCTCTGCACCCAGTTTTC),
and the RPL13A gene (RPL13A For:
CCTGGAGGAGAAGAGGAAAGAGA;
RPL13A Rev:
TTGAGGACCTCTGTGTATTTGTCAA).

Transfections for IL17A Treatment Studies

HEK293T cells were seeded 5×10⁵ cells/well (in 6 well plate) or 3×10⁶ cell/10 cm² plates. Next day, 2 μg or 10 μg of plasmids was transfected using X-tremeGENE 9 DNA Transfection Reagent (Roche) in 6 well plate or 10 cm² plates respectively. For IL-17A (Millipore-Sigma, SRP0675) incubation in cells, 0.5 μg of IL-17A was treated either pre- or post-transfection and incubated at 37° C. After 48 hours, cells were collected by trypsinization. For IL-17A incubation with cell lysates, transfected cell lysates were incubated with presence of 0.5 and 5 μg/ml IL-17A at 4° C. on rotation overnight. Plasmids pLVX-EF1alpha-SARS-CoV-2-orf8-2×Strep-IRES-Puro (Orf8) and pLVX-EF1alpha-eGFP-2×Strep-IRES-Puro (EGFP-Strep) were a gift from Nevan Krogan. (Addgene plasmid #141390, 141395) (Gordon, et al. A SARS-CoV-2 protein interaction map reveals targets for drug repurposing. Nature (2020)). pLVX-EF1alpha-IRES-Puro (Vector) was obtained from Takara/Clontech.

SARS-CoV-2 Orf8 and IL17RA Co-Immunoprecipitation

Transfected and treated HEK293T cells were pelleted and washed in cold D-PBS and later resuspended in Flag-IP Buffer (50 mM Tris HCl, pH 7.4, with 150 mM NaCl, 1 mM EDTA, and 1% NP-40) with 1×HALT (ThermoFisher Scientific, 78429), incubated with buffer for 15 minutes on ice then centrifuged at 13,000 rpm for 5 minutes. The supernatant was collected and 1 mg of protein was used for Immunoprecipitation (IP) with 100 μl Streptactin Sepharose (IBA, 2-1201-010) on a rotor overnight at 4° C. Immunoprecipitates were washed 5 times with Flag-IP buffer and eluted with 1×Buffer E (100 mM Tris-Cl, 150 mM NaCl, 1 mM EDTA, 2.5 mM Desthiobiotin). Eluate was diluted with 1×-NuPAGE (ThermoFisher Scientific, #NP0008) LDS Sample Buffer with 2.5% β-Mercaptoethanol and blotted for targeted antibodies. Antibodies used were Strep Tag II (Qiagen, #34850), B-Actin (Sigma, #A5316), and IL17RA (Cell Signaling, #12661S).

Computational Docking of mPGES-2 and Nsp7

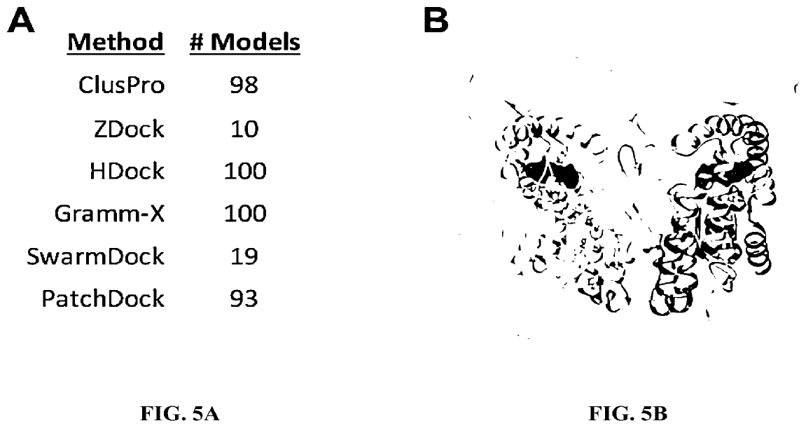

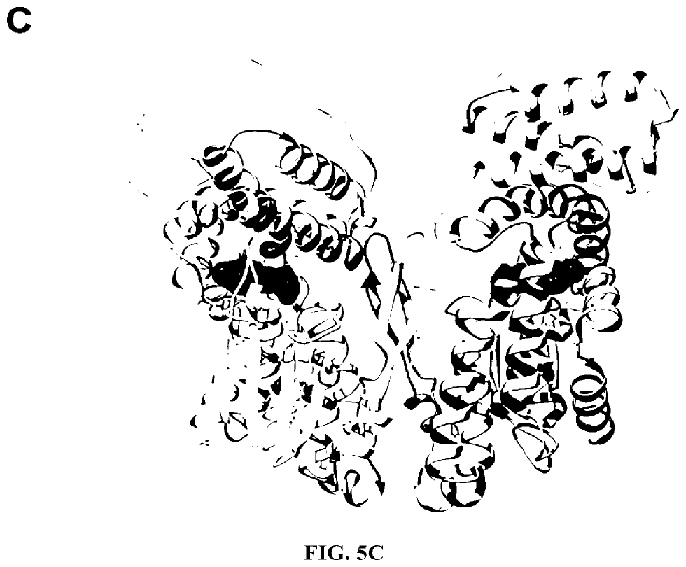

A model for human mPGES-2 dimer was constructed by homology using MODELER (A. Sali, T. L. Blundell, Comparative protein modelling by satisfaction of spatial restraints. J. Mol. Biol. 234, 779-815 (1993)) from the crystal structure of Macaca fascularis mPGES-2 (PDB 1Z9H (T. Yamada, et al., Crystal structure and possible catalytic mechanism of microsomal prostaglandin E synthase type 2 (mPGES-2). J. Mol. Biol. 348, 1163-1176 (2005)), 98% sequence identity) bound to indomethacin. Indomethacin was removed from the structure utilized for docking. The structure of SARS-CoV-2 Nsp7 was extracted from PDB 7BV2 (W. Yin, et al., Structural basis for inhibition of the RNA-dependent RNA polymerase from SARS-CoV-2 by remdesivir. Science. 368, 1499-1504 (2020)). Docking models were produced using ClusPro (D. Kozakov, et al., The ClusPro web server for protein-protein docking. Nat. Protoc. 12, 255-278 (2017)), Zdock (B. G. Pierce, et al., ZDOCK server: interactive docking prediction of protein-protein complexes and symmetric multimers. Bioinformatics. 30, 1771-1773 (2014)), Hdock (Y. Yan, et al., The HDOCK server for integrated protein-protein docking. Nat. Protoc. 15, 1829-1852 (2020)), Gramm-X (A. Tovchigrechko, I. A. Vakser, GRAMM-X public web server for protein-protein docking. Nucleic Acids Res. 34, W310-4 (2006)), SwarmDock (M. Torchala, I. H. Moal, R. A. G. Chaleil, J. Fernandez-Recio, P. A. Bates, SwarmDock: a server for flexible protein-protein docking. Bioinformatics. 29, 807-809 (2013)) and PatchDock (D. Schneidman-Duhovny, et al., PatchDock and SymmDock: servers for rigid and symmetric docking. Nucleic Acids Res. 33, W363-7 (2005)) with SOAP-PP score (G. Q. Dong, et al., Optimized atomic statistical potentials: assessment of protein interfaces and loops. Bioinformatics. 29, 3158-3166 (2013)). For each protocol, up to 100 top scoring models were extracted (fewer for those that do not report>100 models); for PatchDock, models with SOAP-PP Z-scores greater than 3.0 were used (FIG. 5A). The 420 models were clustered at 4.0 Å RMSD, resulting in 127 clusters. The two largest clusters, comprising 192 models, are related by the dimer symmetry. All other clusters contain fewer than 15 models.

Referring to FIG. 5A, the structure of Nsp7 was docked against a homology model of the mPGES-2 dimer (yellow and pink) using a number of docking programs. The number of good scoring models produced by each docking protocol is shown.

Referring to FIG. 5B, the combined localization density of all 420 good scoring models is shown.

Referring to FIG. 5C, the top two clusters of solutions (cyan volume) are symmetry-related and localize to the lobe of mPGES-2 adjacent to the indomethacin binding site (red). Ribbon models of the top scoring models from PatchDock (left) and ZDock (right) represent the two distinct binding modes contained in this cluster of solutions.

Assessment of Positive Selection Signatures in SIGMAR1

SIGMAR1 protein alignments were generated from whole genome sequences of 359 mammals curated by the Zoonomia consortium. Protein alignments were generated with TOGA (https://github.com/hillerlab/TOGA), and missing sequence gaps were refined with CACTUS (J. Armstrong, et al., Progressive alignment with Cactus: a multiple-genome aligner for the thousand-genome era (2019), p. 730531; B. Paten, et al., Cactus: Algorithms for genome multiple sequence alignment. Genome Res. 21, 1512-1528 (2011)). Branches undergoing positive selection were detected with the branch-site test aBSREL (M. D. Smith, et al., Less Is More: An Adaptive Branch-Site Random Effects Model for Efficient Detection of Episodic Diversifying Selection. Mol. Biol. Evol. 32, 1342-1353 (2015)) implemented in the HyPhy package (M. D. Smith, et al., Less Is More: An Adaptive Branch-Site Random Effects Model for Efficient Detection of Episodic Diversifying Selection. Mol. Biol. Evol. 32, 1342-1353 (2015); S. L. K. Pond, et al., HyPhy: hypothesis testing using phylogenies. Bioinformatics. 21, 676-679 (2004)). PhyloP was used to detect codons undergoing accelerated evolution along branches detected as undergoing positive selection by aBSREL relative to the neutral evolution rate in mammals, determined using phyloFit on third nucleotide positions of codons which are assumed to evolve neutrally. P-values from phyloP were corrected for multiple tests using the Benjamini-Hochberg method (K. S. Pollard, et al., Detection of nonneutral substitution rates on mammalian phylogenies. Genome Res. 20, 110-121 (2010)). PhyloFit and phyloP are both part of the PHAST package v1.4 (M. J. Hubisz, et al., PHAST and RPHAST: phylogenetic analysis with space/time models. Brief Bioinform. 12, 41-51 (2011); R. Ramani, et al., PhastWeb: a web interface for evolutionary conservation scoring of multiple sequence alignments using phastCons and phyloP. Bioinformatics. 35, 2320-2322 (2019)).

Comparative SARS-CoV-1 Inhibition by Amiodarone

SARS-CoV-1 (Urbani) drug screens were performed with Vero E6 cells (ATCC #1568, Manassas, VA) cultured in DMEM (Quality Biological), supplemented with 10% (v/v) heat inactivated fetal bovine serum (Sigma), 1% (v/v) penicillin/streptomycin (Gemini Bio-products), and 1% (v/v) L-glutamine (2 mM final concentration, Gibco). Cells were plated in opaque 96 well plates one day prior to infection. Drugs were diluted from stock to 50 μM and an 8-point 1:2 dilution series prepared in duplicate in Vero Media. Every compound dilution and control was normalized to contain the same concentration of drug vehicle (e.g., DMSO). Cells were pre-treated with drug for 2 hours (h) at 37° C. (5% CO₂) prior to infection with SARS-CoV-1 at MOI 0.01. In addition to plates that were infected, parallel plates were left uninfected to monitor cytotoxicity of drug alone. All plates were incubated at 37° C. (5% CO₂) for 3 days before performing CellTiter-Glo (CTG) assays as per the manufacturer's instruction (Promega, Madison, WI). Luminescence was read on a BioTek Synergy HTX plate reader (BioTek Instruments Inc., Winooski, VT) using the Gen5 software (v7.07, Biotek Instruments Inc., Winooski, VT).

Real-World Data Source and Analysis

This study used de-identified patient-level records from HealthVerity's Marketplace dataset, a nationally representative dataset covering>300 million unique patients with medical and pharmacy records from over 60 healthcare data sources in the US. The current study used data from 738,933 patients with documented COVID-19 infection between Mar. 1, 2020 to Aug. 17, 2020, defined as a positive or presumptive positive viral lab test result or an International Classification of Diseases, 10^{th} Revision, Clinical Modification (ICD-10-CM) diagnosis code of U07.1 (COVID-19).

For this population, medical claims, pharmacy claims, laboratory data, and hospital chargemaster data containing diagnoses, procedures, medications, and COVID-19 laboratory results from both inpatient and outpatient settings were analyzed. Claims data included open (unadjudicated) claims sourced in near-real time from practice management and billing systems, claims clearinghouses and laboratory chains, as well as closed (adjudicated) claims encompassing all major US payer types (commercial, Medicare, Medicaid). For inpatient treatment evaluations, linked hospital chargemaster data containing records of all billable procedures, medical services, and treatments administered in hospital settings were used. Linkage of patient-level records across these data types provides a longitudinal view of baseline health status, medication use, and COVID-19 progression for each patient under study. Data for this study covered the period of Dec. 1, 2018 through Aug. 17, 2020. All analyses were conducted with the Aetion Evidence Platform version r4.6.

This study was approved by the New England IRB (#1-9757-1). Medical records constitute protected health information and can be made available to qualified individuals upon reasonable request.

Observation of Hospitalization Outcomes in Outpatient New Users of Indomethacin (Treatment Arm) Vs. Celecoxib (Active Comparator) Using Real-World Data

An incident (new) user, active comparator design (W. A. Ray, Evaluating medication effects outside of clinical trials: new-user designs. Am. J. Epidemiol. 158, 915-920 (2003); S. Schneeweiss, A basic study design for expedited safety signal evaluation based on electronic healthcare data. Pharmacoepidemiol. Drug Saf 19, 858-868 (2010)) was used to assess the risk of hospitalization among newly diagnosed COVID-19 patients who were subsequently treated with indomethacin or the comparator agent, celecoxib. Patients were required to have COVID-19 infection recorded in an outpatient setting during the study period of Mar. 1, 2020 to Aug. 17, 2020 and occurring in the 21 days prior to (and including) the date of indomethacin or celecoxib treatment initiation. Prevalent users of prescription-only NSAIDs (any prescription fill for indomethacin, celecoxib, ketoprofen, meloxicam, sulindac, or piroxicam 60 days prior) and patients hospitalized in the 21 days prior to and including the date of treatment initiation were excluded from this analysis.

Using RSS, patients treated with indomethacin were matched at a 1:1 ratio to controls randomly selected among patients treated with celecoxib, with direct matching on calendar date of treatment (±7 days), age (±5 years), sex, Charlson comorbidity index (exact) (H. Quan, et al., Coding algorithms for defining comorbidities in ICD-9-CM and ICD-10 administrative data. Med. Care. 43, 1130-1139 (2005)), time since confirmed COVID-19 (±5 days), and disease severity based on the highest-intensity COVID-19-related health service in the 7 days prior to and including the date of treatment initiation (lab service only vs. outpatient medical visit vs. emergency department visit) and symptom profile in the 21 days prior to and including the date of treatment initiation (recorded symptoms vs. none). This risk set sampled population was further matched on a propensity score (PS) (P. R. Rosenbaum, D. B. Rubin, The central role of the propensity score in observational studies for causal effects. Biometrika. 70, 41-55 (1983)) estimated using logistic regression with 24 demographic and clinical risk factors, including covariates related to baseline medical history and COVID-19 severity in the 21 days prior to treatment (see Table 7A-I). Balance between indomethacin and celecoxib treatment groups was evaluated by comparison of absolute standardized differences in covariates, with an absolute standardized difference of less than 0.2 indicating good balance between the treatment groups (P. C. Austin, Balance diagnostics for comparing the distribution of baseline covariates between treatment groups in propensity-score matched samples. Stat. Med. 28, 3083-3107 (2009)).

The primary analysis was an intention-to-treat design, with follow-up beginning 1 day after indomethacin or celecoxib initiation and ending on the earliest occurrence of 30 days of follow-up reached or end of patient data. Odds ratios for the primary outcome of all-cause inpatient hospitalization were estimated for the RSS+PS matched population as well as for the RSS matched population. The primary outcome definition required a record of inpatient hospital admission with a resulting inpatient stay; as a sensitivity, a broader outcome definition captured any hospital visit (defined with revenue and place of service codes).

TABLE 7A
TABLE OF CONTENTS

| Table 3B-I Name | Description |
|---|---|
| Data dictionary | Description of all column headings |
| NSAID matching | Matching criteria and cohort |
| | values for the comparison of |
| | new, outpatient users of |
| | indomethacin and celecoxib |
| NSAID cohort | Absolute standard differences |
| balance | of the propensity score risk |
| | factors for the RSS-only and |
| | RSS-and-PS-matched |
| | comparisons of new, outpatient |
| | users of indomethacin and |
| | celecoxib |
| NSAID outcomes | Outcomes of the comparisons |
| | of new, outpatient users of |
| | indomethacin and celecoxib. |
| | Computed by the Action |
| | Evidence Platform r4.6 |
| AP matching | Matching criteria and cohort |
| | values for the comparison of |
| | new, inpatient users of typical |
| | and atypical antipsychotics |
| AP cohort balance | Absolute standard differences |
| | of the propensity score risk |
| | factors for the RSS-only |
| | and RSS-and-PS-matched |
| | comparisons of new, inpatient |
| | users of typical and atypical |
| | antipsychotics |
| AP outcomes | Outcomes of the comparisons |
| | of new, inpatient users of |
| | typical and atypical antipsychotics. |
| | Computed by the Action |
| | Evidence Platform r4.6 |
| Drug list | table of drugs included in |
| | clinical comparisons |

TABLE 7B
DATA DICTIONARY

| Column name | Description |
|---|---|
| Characteristic | Demographic or clinical factor |
| | assessed in patients for |
| | matching |
| Category | Type of risk factor |
| Time period assessed | Time period assessed in records |
| | to determine value of |
| | indicated factor |
| Used for RSS | Boolean variable indicating the |
| matching | use of this characteristic in risk |
| | set sampling |
| Criteria for RSS | Description of matching requirements |
| match | |
| Used for PS matching | Boolean variable indicating the |
| | use of this characteristic in |
| | propensity score matching |
| Criteria for PS match | Description of data type used in |
| | propensity score matching |
| Value and indicated | For a given RSS-only matched |
| distribution in RSS | cohort of users of XXXX drug |
| only XXXX cohort | with number of members YYYY, |
| (n = YYY) | the number of patients in |
| | cohort with a positive identification |
| | for the listed risk factor. |
| | Where appropriate, distribution |
| | as described in the |
| | characteristic column is |
| | included as well. |
| Value and indicated | For a given RSS-and-PS matched |
| distribution in RSS | cohort of users of XXXX |
| and PS XXXX cohort | drug with number of members |
| (n = YYYY) | YYYY, the number of patients |
| | in cohort with a positive identification |
| | for the listed risk factor. |
| | Where appropriate, distribution |
| | as described in the |
| | characteristic column is included as well. |
| Absolute Standard | For the indicated variable, the |
| Difference (RSS | absolute standard difference |
| only) | between the experimental and |
| | comparator groups of the RSS- |
| | only cohort. Absolute standard |
| | difference is defined here: |
| | https://doi.org/10.1002/sim.3697 |
| Absolute Standard | For the indicated variable, the |
| Difference (RSS and | absolute standard difference |
| PS matched) | between the experimental and |
| | comparator groups of the RSS- |
| | and-PS-matched cohort. Absolute |
| | standard difference is |
| | defined here: |
| | https://doi.org/10.1002/sim.3697 |
| RSS only XXXX | In results section these headings |
| cohort | indicate the value of a given |
| | variable for the RSS-only cohort |
| | defined by use of drug |
| | XXXX |
| RSS and PS XXXX | In results section these headings |
| cohort | indicate the value of a given |
| | variable for the RSS-and-PS-matched |
| | cohort defined by use of |
| | drug XXXX |

TABLE 7C
NSAID MATCHING

| | | | | | | | Value and | | Value and | | Value and | | Value and | |
|---|---|---|---|---|---|---|---|---|---|---|---|---|---|---|
| | | | | | | | indicated | | indicated | | indicated | | indicated | |
| | | | | | | | distribution | | distribution | | distribution | | distribution | |
| | | | | | | | in RSS | | in RSS | | in RSS | | in RSS | |
| | | | | | | | only | | only | | and PS | | and PS | |
| | | Time | Used for | | Used for | Criteria | indomethacin | | celecoxib | | indomethacin | | celecoxib | |
| | | period | RSS | Criteria for | PS | for PS | cohort | | cohort | | cohort | | cohort | |
| Characteristic | Category | assessed | matching | RSS match | matching | match | (n = 153) | | (n = 153) | | (n = 103) | | (n = 103) | |
| Month of | Demographic | Date of | Yes | Direct (1:1) | | | | | | | | | | |
| treatment | | treatment | | matching on | | | | | | | | | | |
| initiation | | initiation | | calendar date | | | | | | | | | | |
| | | | | of treatment | | | | | | | | | | |
| | | | | initiation, | | | | | | | | | | |
| | | | | +/−7 days | | | | | | | | | | |
| . . . March/ | Demographic | Date of | — | — | — | — | 58 | (37.9%) | 58 | (37.9%) | 34 | (33.0%) | 34 | (33.0%) |
| April 2020 | | treatment | | | | | | | | | | | | |
| | | initiation | | | | | | | | | | | | |
| . . . May | Demographic | Date of | — | — | — | — | 50 | (32.7%) | 51 | (33.3%) | 35 | (34.0%) | 34 | (33.0%) |
| 2020 | | treatment | | | | | | | | | | | | |
| | | initiation | | | | | | | | | | | | |
| . . . June | Demographic | Date of | — | — | — | — | 22 | (14.4%) | 21 | (13.7%) | 17 | (16.5%) | 16 | (15.5%) |
| 2020 | | treatment | | | | | | | | | | | | |
| | | initiation | | | | | | | | | | | | |
| . . . July/ | Demographic | Date of | — | — | — | — | 23 | (15.0%) | 23 | (15.0%) | 17 | (16.5%) | 19 | (18.4%) |
| August | | treatment | | | | | | | | | | | | |
| 2020 | | initiation | | | | | | | | | | | | |
| Age | Demographic | Date of | Yes | Direct (1:1) | Yes | Age as | | | | | | | | |
| | | treatment | | matching on | | continuous | | | | | | | | |
| | | initiation | | age, +/−5 | | numeric | | | | | | | | |
| | | | | years | | variable | | | | | | | | |
| . . . mean | Demographic | Date of | — | — | — | — | 52.88 | (11.65) | 53.24 | (12.07) | 53.74 | (11.89) | 52.95 | (12.72) |
| (sd) | | treatment | | | | | | | | | | | | |
| | | initiation | | | | | | | | | | | | |
| . . . median | Demographic | Date of | — | — | — | — | 54 | [46, 61] | 54 | [46.50, 62] | 54 | [47, 61] | 55 | [46, 63] |
| [IQR] | | treatment | | | | | | | | | | | | |
| | | initiation | | | | | | | | | | | | |
| Gender | Demographic | Date of | Yes | Direct (1:1) | Yes | Categorical | | | | | | | | |
| | | treatment | | matching on | | | | | | | | | | |
| | | initiation | | gender | | | | | | | | | | |
| . . . Female | Demographic | Date of | — | — | — | — | 65 | (42.5%) | 65 | (42.5%) | 41 | (39.8%) | 50 | (48.5%) |
| | | treatment | | | | | | | | | | | | |
| | | initiation | | | | | | | | | | | | |
| . . . Male | Demographic | Date of | — | — | — | — | 88 | (57.5%) | 88 | (57.5%) | 62 | (60.2%) | 53 | (51.5%) |
| | | treatment | | | | | | | | | | | | |
| | | initiation | | | | | | | | | | | | |
| U.S. | Demographic | Date of | No | — | Yes | Categorical | | | | | | | | |
| Region | | treatment | | | | | | | | | | | | |
| | | initiation | | | | | | | | | | | | |
| . . . Northeast | Demographic | Date of | — | — | — | — | 68 | (44.4%) | 74 | (48.4%) | 46 | (44.7%) | 48 | (46.6%) |
| | | treatment | | | | | | | | | | | | |
| | | initiation | | | | | | | | | | | | |
| . . . Midwest/ | Demographic | Date of | — | — | — | — | 43 | (28.1%) | 40 | (26.1%) | 29 | (28.2%) | 27 | (26.2%) |
| West | | treatment | | | | | | | | | | | | |
| | | initiation | | | | | | | | | | | | |
| . . . South | Demographic | Date of | — | — | — | — | 42 | (27.5%) | 39 | (25.5%) | 28 | (27.2%) | 28 | (27.2%) |
| | | treatment | | | | | | | | | | | | |
| | | initiation | | | | | | | | | | | | |
| No. of | Baseline | 90 days | No | | Yes | Continuous | | | | | | | | |
| medical | health | prior to | | | | numeric | | | | | | | | |
| encounters | resource | date of | | | | variable | | | | | | | | |
| | utilization | confirmed | | | | | | | | | | | | |
| | | COVID19 | | | | | | | | | | | | |
| . . . mean | Baseline | 90 days | — | — | — | — | 4.78 | (4.63) | 6.88 | (9.02) | 4.71 | (4.78) | 4.71 | (4.35) |
| (sd) | health | prior to | | | | | | | | | | | | |
| | resource | date of | | | | | | | | | | | | |
| | utilization | confirmed | | | | | | | | | | | | |
| | | COVID19 | | | | | | | | | | | | |
| . . . median | Baseline | 90 days | — | — | — | — | 3 | [2, 6] | 4 | [2, 8] | 3 | [1, 6] | 3 | [2, 6] |
| [IQR] | health | prior to | | | | | | | | | | | | |
| | resource | date of | | | | | | | | | | | | |
| | utilization | confirmed | | | | | | | | | | | | |
| | | COVID19 | | | | | | | | | | | | |
| No. of | Baseline | 90 days | No | | Yes | Continuous | | | | | | | | |
| pharmacy | health | prior to | | | | numeric | | | | | | | | |
| claims | resource | date of | | | | variable | | | | | | | | |
| | utilization | confirmed | | | | | | | | | | | | |
| | | COVID19 | | | | | | | | | | | | |
| . . . mean | Baseline | 90 days | — | — | — | — | 5.97 | (5.04) | 6.92 | (5.41) | 6.25 | (5.47) | 6.25 | (4.82) |
| (sd) | health | prior to | | | | | | | | | | | | |
| | resource | date of | | | | | | | | | | | | |
| | utilization | confirmed | | | | | | | | | | | | |
| | | COVID19 | | | | | | | | | | | | |
| . . . median | Baseline | 90 days | — | — | — | — | 5 | [3, 7.50] | 6 | [3, 9] | 5 | [3, 8] | 5 | [3, 8] |
| [IQR] | health | prior to | | | | | | | | | | | | |
| | resource | date of | | | | | | | | | | | | |
| | utilization | confirmed | | | | | | | | | | | | |
| | | COVID19 | | | | | | | | | | | | |
| No. of | Baseline | 90 days | No | | Yes | Continuous | | | | | | | | |
| unique | health | prior to | | | | numeric | | | | | | | | |
| medications | resource | date of | | | | variable | | | | | | | | |
| dispensed | utilization | confirmed | | | | | | | | | | | | |
| | | COVID19 | | | | | | | | | | | | |
| . . . mean | Baseline | 90 days | — | — | — | — | 8.02 | (5.51) | 7.81 | (4.64) | 7.27 | (4.81) | 7.40 | (4.54) |
| (sd) | health | prior to | | | | | | | | | | | | |
| | resource | date of | | | | | | | | | | | | |
| | utilization | confirmed | | | | | | | | | | | | |
| | | COVID19 | | | | | | | | | | | | |
| . . . median | Baseline | 90 days | — | — | — | — | 7 | [4, 11] | 7 | [4.50, 10] | 7 | [3, 10] | 6 | [4, 9] |
| [IQR] | health | prior to | | | | | | | | | | | | |
| | resource | date of | | | | | | | | | | | | |
| | utilization | confirmed | | | | | | | | | | | | |
| | | COVID19 | | | | | | | | | | | | |
| Charlson | Baseline | 90 days | Yes | Direct (1:1) | Yes | Continuous | | | | | | | | |
| comorbidity | comorbidities | prior to | | matching on | | numeric | | | | | | | | |
| index | and | date of | | Charlson | | variable | | | | | | | | |
| | comedications | confirmed | | comorbidity | | | | | | | | | | |
| | | COVID19 | | score in 90 | | | | | | | | | | |
| | | | | days prior, | | | | | | | | | | |
| | | | | categorized | | | | | | | | | | |
| | | | | (0-1, 2-3, 4-5, | | | | | | | | | | |
| | | | | 6+). | | | | | | | | | | |
| . . . mean | Baseline | 90 days | — | — | — | — | 0.36 | (0.82) | 0.43 | (0.81) | 0.38 | (0.90) | 0.32 | (0.56) |
| (sd) | comorbidities | prior to | | | | | | | | | | | | |
| | and | date of | | | | | | | | | | | | |
| | comedications | confirmed | | | | | | | | | | | | |
| | | COVID19 | | | | | | | | | | | | |
| . . . median | Baseline | 90 days | — | — | — | — | 0 | [0, 1] | 0 | [0, 1] | 0 | [0, 1] | 0 | [0, 1] |
| [IQR] | comorbidities | prior to | | | | | | | | | | | | |
| | and | date of | | | | | | | | | | | | |
| | comedications | confirmed | | | | | | | | | | | | |
| | | COVID19 | | | | | | | | | | | | |
| Chronic | Baseline | 90 days | No | — | Yes | Dichotomous | 18 | (11.8%) | 19 | (12.4%) | 11 | (10.7%) | 12 | (11.7%) |
| pulmonary | comorbidities | prior to | | | | | | | | | | | | |
| disease | and | date of | | | | | | | | | | | | |
| | comedications | confirmed | | | | | | | | | | | | |
| | | COVID19 | | | | | | | | | | | | |
| Cardiovascular | Baseline | 90 days | No | — | Yes | Dichotomous | 45 | (29.4%) | 53 | (34.6%) | 32 | (31.1%) | 29 | (28.2%) |
| disease | comorbidities | prior to | | | | | | | | | | | | |
| | and | date of | | | | | | | | | | | | |
| | comedications | confirmed | | | | | | | | | | | | |
| | | COVID19 | | | | | | | | | | | | |
| . . . Arrhythmia | Baseline | 90 days | No | — | Yes | Dichotomous | 11 | (7.2%) | 16 | (10.5%) | 10 | (9.7%) | 10 | (9.7%) |
| | comorbidities | prior to | | | | | | | | | | | | |
| | and | date of | | | | | | | | | | | | |
| | comedications | confirmed | | | | | | | | | | | | |
| | | COVID19 | | | | | | | | | | | | |
| . . . Hyper- | Baseline | 90 days | No | — | Yes | Dichotomous | 63 | (41.2%) | 76 | (49.7%) | 45 | (43.7%) | 44 | (42.7%) |
| ension | comorbidities | prior to | | | | | | | | | | | | |
| | and | date of | | | | | | | | | | | | |
| | comedications | confirmed | | | | | | | | | | | | |
| | | COVID19 | | | | | | | | | | | | |
| Diabetes | Baseline | 90 days | No | — | Yes | Dichotomous | 24 | (15.7%) | 28 | (18.3%) | 17 | (16.5%) | 17 | (16.5%) |
| | comorbidities | prior to | | | | | | | | | | | | |
| | and | date of | | | | | | | | | | | | |
| | comedications | confirmed | | | | | | | | | | | | |
| | | COVID19 | | | | | | | | | | | | |
| Immuno- | Baseline | 90 days | No | — | Yes | Dichotomous | 35 | (22.9%) | 28 | (18.3%) | 20 | (19.4%) | 19 | (18.4%) |
| suppressive | comorbidities | prior to | | | | | | | | | | | | |
| condition | and | date of | | | | | | | | | | | | |
| | comedications | confirmed | | | | | | | | | | | | |
| | | COVID19 | | | | | | | | | | | | |
| Any | Baseline | 90 days | No | — | Yes | Dichotomous | 8 | (5.2%) | 6 | (3.9%) | 4 | (3.9%) | 3 | (2.9%) |
| respiratory | comorbidities | prior to | | | | | | | | | | | | |
| support | and | date of | | | | | | | | | | | | |
| or | comedications | confirmed | | | | | | | | | | | | |
| supplemental | | COVID19 | | | | | | | | | | | | |
| oxygen | | | | | | | | | | | | | | |
| use | | | | | | | | | | | | | | |
| Tobacco | Baseline | 90 days | No | — | Yes | Dichotomous | 7 | (4.6%) | 17 | (11.1%) | 6 | (5.8%) | 5 | (4.9%) |
| use | comorbidities | prior to | | | | | | | | | | | | |
| recorded | and | date of | | | | | | | | | | | | |
| | comedications | confirmed | | | | | | | | | | | | |
| | | COVID19 | | | | | | | | | | | | |
| Kidney | Baseline | 90 days | No | — | Yes | Dichotomous | 5 | (3.3%) | 4 | (2.6%) | 4 | (3.9%) | 2 | (1.9%) |
| or liver | comorbidities | prior to | | | | | | | | | | | | |
| disease | and | date of | | | | | | | | | | | | |
| | comedications | confirmed | | | | | | | | | | | | |
| | | COVID19 | | | | | | | | | | | | |
| Overweight | Baseline | 90 days | No | — | Yes | Dichotomous | 27 | (17.6%) | 38 | (24.8%) | 17 | (16.5%) | 19 | (18.4%) |
| or obese | comorbidities | prior to | | | | | | | | | | | | |
| | and | date of | | | | | | | | | | | | |
| | comedications | confirmed | | | | | | | | | | | | |
| | | COVID19 | | | | | | | | | | | | |
| Use of | Baseline | 90 days | No | — | Yes | Dichotomous | 10 | (6.5%) | 11 | (7.2%) | 3 | (2.9%) | 7 | (6.8%) |
| any | comorbidities | prior to | | | | | | | | | | | | |
| antithrombotic | and | date of | | | | | | | | | | | | |
| therapy | comedications | confirmed | | | | | | | | | | | | |
| | | COVID19 | | | | | | | | | | | | |
| Use of | Baseline | 90 days | No | — | Yes | Dichotomous | 37 | (24.2%) | 47 | (30.7%) | 31 | (30.1%) | 27 | (26.2%) |
| statin | comorbidities | prior to | | | | | | | | | | | | |
| medication | and | date of | | | | | | | | | | | | |
| | comedications | confirmed | | | | | | | | | | | | |
| | | COVID19 | | | | | | | | | | | | |
| Use of | Baseline | 90 days | No | — | Yes | Dichotomous | 39 | (25.5%) | 46 | (30.1%) | 29 | (28.2%) | 26 | (25.2%) |
| any | comorbidities | prior to | | | | | | | | | | | | |
| steroid | and | date of | | | | | | | | | | | | |
| medication | comedications | confirmed | | | | | | | | | | | | |
| | | COVID19 | | | | | | | | | | | | |
| Symptom | COVID19 | 21 days | Yes | Direct (1:1) | Yes | Dichotomous, | 32 | (20.9%) | 34 | (22.2%) | 20 | (19.4%) | 20 | (19.4%) |
| profile, | severity and | prior to | | matching on | | moderate | | | | | | | | |
| moderate | utilization | treatment | | symptom | | to severe | | | | | | | | |
| to severe | | initiation | | profile in 21 | | COVID- | | | | | | | | |
| symptoms | | (inclusive) | | days pre- | | 19 signs or | | | | | | | | |
| | | | | treatment, | | symptoms | | | | | | | | |
| | | | | symptomatic | | | | | | | | | | |
| | | | | VS | | | | | | | | | | |
| | | | | asymptomatic. | | | | | | | | | | |
| | | | | Note this RSS | | | | | | | | | | |
| | | | | matching | | | | | | | | | | |
| | | | | criteria uses a | | | | | | | | | | |
| | | | | broader set of | | | | | | | | | | |
| | | | | all possible | | | | | | | | | | |
| | | | | signs and | | | | | | | | | | |
| | | | | symptoms, | | | | | | | | | | |
| | | | | whereas the | | | | | | | | | | |
| | | | | PS inputs and | | | | | | | | | | |
| | | | | results shown | | | | | | | | | | |
| | | | | in columns H- | | | | | | | | | | |
| | | | | K use a | | | | | | | | | | |
| | | | | narrower | | | | | | | | | | |
| | | | | definition. | | | | | | | | | | |
| Time | COVID19 | Date of | Yes | Direct (1:1) | Yes | Continuous | | | | | | | | |
| from | severity and | confirmed | | matching on | | numeric | | | | | | | | |
| documented | utilization | COVID19 | | time from | | variable | | | | | | | | |
| COVID19 | | to date | | documented | | | | | | | | | | |
| to drug | | of | | COVID19 | | | | | | | | | | |
| initiation, | | treatment | | infection to | | | | | | | | | | |
| no. days | | initiation | | treatment | | | | | | | | | | |
| | | (inclusive) | | initiation, | | | | | | | | | | |
| | | | | +/− 5 days | | | | | | | | | | |
| . . . mean | COVID19 | Date of | — | — | — | — | 9.61 | (7.01) | 9.75 | (6.94) | 8.99 | (7.06) | 9.73 | (7.06) |
| (sd) | severity and | confirmed | | | | | | | | | | | | |
| | utilization | COVID19 | | | | | | | | | | | | |
| | | to date | | | | | | | | | | | | |
| | | of | | | | | | | | | | | | |
| | | treatment | | | | | | | | | | | | |
| | | initiation | | | | | | | | | | | | |
| | | (inclusive) | | | | | | | | | | | | |
| . . . median | COVID19 | Date of | — | — | — | — | 8 | [3.50, 15.50] | 9 | [4, 15] | 7 | [2, 15] | 8 | [4, 15] |
| [IQR] | severity and | confirmed | | | | | | | | | | | | |
| | utilization | COVID19 | | | | | | | | | | | | |
| | | to date | | | | | | | | | | | | |
| | | of | | | | | | | | | | | | |
| | | treatment | | | | | | | | | | | | |
| | | initiation | | | | | | | | | | | | |
| | | (inclusive) | | | | | | | | | | | | |
| Any | COVID19 | 21 days | No | — | Yes | Dichotomous | 39 | (25.5%) | 40 | (26.1%) | 23 | (22.3%) | 23 | (22.3%) |

| | | | | | | | | | | |
|---|---|---|---|---|---|---|---|---|---|---|
| emergency | severity and | prior to | | | | | | | | |
| department or | utilization | treatment | | | | | | | | |
| hospital | | initiation | | | | | | | | |
| interaction | | (inclusive) | | | | | | | | |
| COVID19 | COVID19 | 7 days | Yes | Direct (1:1) | No | — | — | — | — | — |
| health | severity and | prior to | | matching on | | | | | | |
| resource | utilization | treatment | | highest | | | | | | |
| utilization | | initiation | | recorded | | | | | | |
| | | (inclusive) | | health | | | | | | |
| | | | | resource | | | | | | |
| | | | | utilization in | | | | | | |
| | | | | the 7 days | | | | | | |
| | | | | prior | | | | | | |
| | | | | (inclusive), | | | | | | |
| | | | | categorized | | | | | | |
| | | | | (laboratory | | | | | | |
| | | | | only, | | | | | | |
| | | | | outpatient | | | | | | |
| | | | | medical visit, | | | | | | |
| | | | | emergency | | | | | | |
| | | | | department or | | | | | | |
| | | | | hospital | | | | | | |
| | | | | encounter) | | | | | | |

TABLE 7D
NSAID COHORT BALANCE

| | Absolute | Absolute |
|---|---|---|
| | Standard | Standard |
| | Difference | Difference |
| | (RSS | (RSS and |
| Variable | only) | PS matched) |
| Month of treatment initiation | 0.021 | 0.055 |
| Age | 0.030 | 0.064 |
| Gender | 0.000 | 0.177 |
| U.S. Region | 0.079 | 0.047 |
| No. of medical encounters | 0.294 | 0.000 |
| No. of pharmacy claims | 0.180 | 0.000 |
| No. of unique medications dispensed | 0.041 | 0.027 |
| Charlson comorbidity index | 0.088 | 0.078 |
| Chronic pulmonary disease | 0.020 | 0.031 |
| Cardiovascular disease (any) | 0.112 | 0.064 |
| . . . Arrhythmia | 0.115 | 0.000 |
| . . . Hypertension | 0.171 | 0.020 |
| Diabetes | 0.070 | 0.000 |
| Immunosuppressive condition | 0.113 | 0.025 |
| Any respiratory support or | 0.063 | 0.054 |
| supplemental oxygen use | | |
| Positive tobacco user | 0.245 | 0.043 |
| Kidney or liver disease | 0.039 | 0.116 |
| Overweight or obese | 0.176 | 0.051 |
| Use of any antithrombotic therapy | 0.026 | 0.181 |
| Use of statin medication | 0.147 | 0.086 |
| Use of any steroid medication | 0.102 | 0.066 |
| Moderate to severe COVID-19 | 0.032 | 0.000 |
| signs or symptoms | | |
| Time from documented COVID19 | 0.019 | 0.105 |
| to drug initiation, no. days | | |
| Any emergency department or hospital | 0.015 | 0.000 |
| interaction, 21 days prior | | |
| Average standardized absolute | 0.092 | 0.054 |
| mean difference | | |

TABLE 7E
NSAID OUTCOMES

| | RSS | | RSS | |
|---|---|---|---|---|
| | only | RSS | and PS | RSS |
| | indo- | only | indo- | and PS |
| | methacin | celecoxib | methacin | celecoxib |
| Cohort | cohort | cohort | cohort | cohort |
| Treatment | indo- | celecoxib | indo- | celecoxib |
| | methacin | | methacin | |
| Treatment classification | Experi- | Referent | Experi- | Referent |
| | mental | | mental | |
| Matching criteria | RSS | RSS | RSS | RSS |
| | only | only | and PS | and PS |
| Number of patients | 153 | 153 | 103 | 103 |
| Number of confirmed | 1 | 7 | 1 | 3 |
| inpatient stays | | | | |
| Risk of confirmed | 6.54 | 45.75 | 9.71 | 29.13 |
| inpatient stays per | | | | |
| 1000 patients | | | | |
| Risk ratio vs referent | 0.14 | NA | 0.33 | NA |
| of confirmed | | | | |
| inpatient stay | | | | |
| 95% confidence | 0.02 | NA | 0.04, 3.15 | NA |
| interval of risk ratio vs | | | | |
| referent of confirmed | | | | |
| outpatient stay, | | | | |
| lower bound | | | | |
| 95% confidence | 1.15 | NA | 0.04, 3.15 | NA |
| interval of risk ratio vs | | | | |
| referent of confirmed | | | | |
| outpatient stay, | | | | |
| upper bound | | | | |
| Odds ratio of confirmed | 0.14 | NA | 0.33 | NA |
| inpatient stay | | | (0.04, | |
| versus referent | | | 3.15) | |
| 95% confidence | 0.02 | NA | 0.04 | NA |
| interval of odds ratio of | | | | |
| confirmed inpatient | | | | |
| stay versus referent, | | | | |
| lower bound | | | | |
| 95% confidence | 1.13 | NA | 3.15 | NA |
| interval of odds ratio of | | | | |
| confirmed inpatient | | | | |
| stay versus referent, | | | | |
| upper bound | | | | |
| p-value of odds | 0.065 | NA | 0.336 | NA |
| ratio of confirmed | | | | |
| inpatient stay versus referent | | | | |
| Number of patients | 4 | 15 | 3 | 7 |
| with any hospital visit | | | | |
| Risk of any hospital | 26.14 | 98.04 | 29.13 | 67.96 |
| visit per 1000 | | | | |
| patients | | | | |
| Risk ratio vs referent | 0.27 | NA | 0.43 | NA |
| of any hospital visit | | | | |
| 95% confidence | 0.09 | NA | 0.11 | NA |
| interval of risk ratio vs | | | | |
| referent of any hospital | | | | |
| visit, lower bound | | | | |
| 95% confidence | 0.79 | NA | 1.61 | NA |
| interval of risk ratio vs | | | | |
| referent of any hospital | | | | |
| visit, upper bound | | | | |
| Odds ratio of any | 0.25 | NA | 0.41 | NA |
| hospital visit versus | | | | |
| referent | | | | |
| 95% confidence | 0.08 | NA | 0.1 | NA |
| interval of odds ratio of | | | | |
| any hospital visit | | | | |
| versus referent, lower | | | | |
| bound | | | | |
| 95% confidence | 0.76 | NA | 1.64 | NA |
| interval of odds ratio of | | | | |
| any hospital visit | | | | |
| versus referent, upper | | | | |
| bound | | | | |
| p-value of odds ratio of any | 0.015 | NA | 0.208 | NA |
| hospital visit versus referent | | | | |

TABLE 7F
AP MATCHING

| | | | | | | | Value and | Value and | Value and | Value and |
|---|---|---|---|---|---|---|---|---|---|---|
| | | | | | | | indicated | indicated | indicated | indicated |
| | | | | | | | distribution | distribution | distribution | distribution |
| | | | | | | | in RSS only | in RSS only | in RSS and | in RSS and |
| | | | Used | | Used | Criteria | typical | atypical | PS typical | PS atypical |
| | | Time period | for RSS | | for PS | for PS | AP cohort | AP cohort | AP cohort | AP cohort |
| Characteristic | Category | assessed | matching | Criteria for RSS match | matching | match | (n = 265) | (n = 265) | (n = 186) | (n = 186) |
| Month of | Demographic | Date of treatment | Yes | Direct (1:1) matching on calendar date | Yes | Categorical | | | | |
| treatment | | initiation | | of treatment initiation, +/−7 days | | | | | | |
| initiation | | | | | | | | | | |
| . . . March/April 2020 | Demographic | Date of treatment | — | — | — | — | 124 (46.8%) | 126 (47.5%) | 77 (41.4%) | 80 (43.0%) |
| | | initiation | | | | | | | | |
| . . . May 2020 | Demographic | Date of treatment | — | — | — | — | 68 (25.7%) | 67 (25.3%) | 47 (25.3%) | 50 (26.9%) |
| | | initiation | | | | | | | | |
| . . . June 2020 | Demographic | Date of treatment | — | — | — | — | 26 (9.8%) | 26 (9.8%) | 22 (11.8%) | 22 (11.8%) |
| | | initiation | | | | | | | | |
| . . . July/Aug 2020 | Demographic | Date of treatment | — | — | — | — | 47 (17.7%) | 46 (17.4%) | 40 (21.5%) | 34 (18.3%) |
| | | initiation | | | | | | | | |
| Age | Demographic | Date of treatment | Yes | Direct (1:1) matching on age, +/−5 | Yes | Age as | | | | |
| | | initiation | | years | | continuous | | | | |
| | | | | | | numeric | | | | |
| | | | | | | variable | | | | |
| . . . mean (sd) | Demographic | Date of treatment | — | — | — | — | 69.93 (17.50) | 69.83 (17.36) | 68.83 (18.33) | 69.19 (17.99) |
| | | initiation | | | | | | | | |
| . . . median [IQR] | Demographic | Date of treatment | — | — | — | — | 72 [61, 82] | 71 [62, 82] | 71 [60, 81.25] | 70 [61.75, 82] |
| | | initiation | | | | | | | | |
| Gender | Demographic | Date of treatment | Yes | Direct (1:1) matching on gender | Yes | Categorical | | | | |
| | | initiation | | | | | | | | |
| . . . Female | Demographic | Date of treatment | — | — | — | — | 106 (40.0%) | 106 (40.0%) | 69 (37.1%) | 69 (37.1%) |
| | | initiation | | | | | | | | |
| . . . Male | Demographic | Date of treatment | — | — | — | — | 159 (60.0%) | 159 (60.0%) | 117 (62.9%) | 117 (62.9%) |
| | | initiation | | | | | | | | |
| U.S. Region | Demographic | Date of treatment | No | — | Yes | Categorical | | | | |
| | | initiation | | | | | | | | |
| . . . Northeast | Demographic | Date of treatment | — | — | — | — | 134 (50.6%) | 116 (43.8%) | 83 (44.6%) | 83 (44.6%) |
| | | initiation | | | | | | | | |
| . . . Midwest/West | Demographic | Date of treatment | — | — | — | — | 54 (20.4%) | 75 (28.3%) | 48 (25.8%) | 47 (25.3%) |
| | | initiation | | | | | | | | |
| . . . South | Demographic | Date of treatment | — | — | — | — | 77 (29.1%) | 74 (27.9%) | 55 (29.6%) | 56 (30.1%) |
| | | initiation | | | | | | | | |
| No. of medical | Baseline | 90 days prior to | No | — | Yes | Continuous | | | | |
| encounters | health | hospitalization, not | | | | numeric | | | | |
| | resource | including date of | | | | variable | | | | |
| | utilization | hospitalization | | | | | | | | |
| . . . mean (sd) | Baseline | 90 days prior to | — | — | — | — | 14.17 (21.51) | 16.08 (23.75) | 15.90 (22.57) | 13.19 (20.39) |
| | health | hospitalization, not | | | | | | | | |
| | resource | including date of | | | | | | | | |
| | utilization | hospitalization | | | | | | | | |
| . . . median [IQR] | Baseline | 90 days prior to | — | — | — | — | 4 [1, 19] | 6 [2, 19] | 5 [1,21] | 5 [1, 16] |
| | health | hospitalization, not | | | | | | | | |
| | resource | including date of | | | | | | | | |
| | utilization | hospitalization | | | | | | | | |
| No. of unique | Baseline | 90 days prior to | No | — | Yes | Continuous | | | | |
| medications | health | hospitalization, not | | | | numeric | | | | |
| dispensed | resource | including date of | | | | variable | | | | |
| | utilization | hospitalization | | | | | | | | |
| . . . mean (sd) | Baseline | 90 days prior to | — | — | — | — | 3.80 (5.21) | 2.63 (4.39) | 3.37 (4.74) | 3.06 (4.77) |
| | health | hospitalization, not | | | | | | | | |
| | resource | including date of | | | | | | | | |
| | utilization | hospitalization | | | | | | | | |
| . . . median [IQR] | Baseline | 90 days prior to | — | — | — | — | 1 [0, 7] | 0 [0, 4] | 1 [0, 6] | 0 [0, 5] |
| | health | hospitalization, not | | | | | | | | |
| | resource | including date of | | | | | | | | |
| | utilization | hospitalization | | | | | | | | |
| Charlson | Baseline | 90 days prior to | Yes | Direct (1:1) matching on Charlson | Yes | Continuous | | | | |
| comorbidity | comorbidities | hospitalization, not | | comorbidity score in 90 days prior, | | numeric | | | | |
| index | and | including date of | | categorized (0-1, 2-3, 4-5, 6+). | | variable | | | | |
| | comedications | hospitalization | | | | | | | | |
| . . . mean (sd) | Baseline | 90 days prior to | — | — | — | — | 1.76 (2.40) | 1.70 (2.19) | 1.80 (2.39) | 1.48 (2.09) |
| | comorbidities | hospitalization, not | | | | | | | | |
| | and | including date of | | | | | | | | |
| | comedications | hospitalization | | | | | | | | |
| . . . median [IQR] | Baseline | 90 days prior to | — | — | — | — | 1 [0, 3] | 1 [0, 3] | 1 [0, 3] | 1 [0, 2] |
| | comorbidities | hospitalization, not | | | | | | | | |
| | and | including date of | | | | | | | | |
| | comedications | hospitalization | | | | | | | | |
| Cancer | Baseline | 90 days prior to | No | — | Yes | Dichotomous | 14 (5.3%) | 15 (5.7%) | 11 (5.9%) | 10 (5.4%) |
| | comorbidities | hospitalization, not | | | | | | | | |
| | and | including date of | | | | | | | | |
| | comedications | hospitalization | | | | | | | | |
| Chronic | Baseline | 90 days prior to | No | — | Yes | Dichotomous | 39 (14.7%) | 57 (21.5%) | 32 (17.2%) | 31 (16.7%) |
| pulmonary | comorbidities | hospitalization, not | | | | | | | | |
| disease | and | including date of | | | | | | | | |
| | comedications | hospitalization | | | | | | | | |
| Cardiovascular | Baseline | 90 days prior to | No | — | Yes | Dichotomous | 145 (54.7%) | 133 (50.2%) | 99 (53.2%) | 91 (48.9%) |
| disease (any) | comorbidities | hospitalization, not | | | | | | | | |
| | and | including date of | | | | | | | | |
| | comedications | hospitalization | | | | | | | | |
| . . . Arrhythmia | Baseline | 90 days prior to | No | — | Yes | Dichotomous | 60 (22.6%) | 49 (18.5%) | 43 (23.1%) | 36 (19.4%) |
| | comorbidities | hospitalization, not | | | | | | | | |
| | and | including date of | | | | | | | | |
| | comedications | hospitalization | | | | | | | | |
| . . . Hypertension | Baseline | 90 days prior to | No | — | Yes | Dichotomous | 153 (57.7%) | 137 (51.7%) | 104 (55.9%) | 100 (53.8%) |
| | comorbidities | hospitalization, not | | | | | | | | |
| | and | including date of | | | | | | | | |
| | comedications | hospitalization | | | | | | | | |
| Dementia | Baseline | 90 days prior to | No | — | Yes | Dichotomous | 60 (22.6%) | 62 (23.4%) | 40 (21.5%) | 34 (18.3%) |
| | comorbidities | hospitalization, not | | | | | | | | |
| | and | including date of | | | | | | | | |
| | comedications | hospitalization | | | | | | | | |
| Diabetes | Baseline | 90 days prior to | No | — | Yes | Dichotomous | 68 (25.7%) | 66 (24.9%) | 47 (25.3%) | 39 (21.0%) |
| | comorbidities | hospitalization, not | | | | | | | | |
| | and | including date of | | | | | | | | |
| | comedications | hospitalization | | | | | | | | |
| Tobacco use | Baseline | 90 days prior to | No | — | Yes | Dichotomous | 37 (14.0%) | 37 (14.0%) | 26 (14.0%) | 25 (13.4%) |
| recorded | comorbidities | hospitalization, not | | | | | | | | |
| | and | including date of | | | | | | | | |
| | comedications | hospitalization | | | | | | | | |
| Kidney or liver | Baseline | 90 days prior to | No | — | Yes | Dichotomous | 58 (21.9%) | 54 (20.4%) | 44 (23.7%) | 37 (19.9%) |
| disease | comorbidities | hospitalization, not | | | | | | | | |
| | and | including date of | | | | | | | | |
| | comedications | hospitalization | | | | | | | | |
| Immunosuppressive | Baseline | 90 days prior to | No | — | Yes | Dichotomous | 38 (14.3%) | 36 (13.6%) | 30 (16.1%) | 23 (12.4%) |
| condition | comorbidities | hospitalization, not | | | | | | | | |
| | and | including date of | | | | | | | | |
| | comedications | hospitalization | | | | | | | | |
| Overweight or | Baseline | 90 days prior to | No | — | Yes | Dichotomous | 30 (11.3%) | 25 (9.4%) | 21 (11.3%) | 20 (10.8%) |
| obese | comorbidities | hospitalization, not | | | | | | | | |
| | and | including date of | | | | | | | | |
| | comedications | hospitalization | | | | | | | | |
| Use of any | Baseline | 90 days prior to | No | — | Yes | Dichotomous | 186 (70.2%) | 204 (77.0%) | 141 (75.8%) | 139 (74.7%) |
| antithrombotic | comorbidities | hospitalization to | | | | | | | | |
| therapy* | and | date of treatment | | | | | | | | |
| | comedications | initiation (includes | | | | | | | | |
| | | both pre-admission | | | | | | | | |
| | | and in-hospital, | | | | | | | | |
| | | pre-treatment | | | | | | | | |
| | | periods). | | | | | | | | |
| Use of statin | Baseline | 90 days prior to | No | — | Yes | Dichotomous | 63 (23.8%) | 38 (14.3%) | 35 (18.8%) | 33 (17.7%) |
| medication | comorbidities | hospitalization, not | | | | | | | | |
| | and | including date of | | | | | | | | |
| | comedications | hospitalization | | | | | | | | |
| Use of any | Baseline | 90 days prior to | No | — | Yes | Dichotomous | 60 (22.6%) | 66 (24.9%) | 47 (25.3%) | 49 (26.3%) |
| steroid | comorbidities | hospitalization to | | | | | | | | |
| medication* | and | date of treatment | | | | | | | | |
| | comedications | initiation (includes | | | | | | | | |
| | | both pre-admission | | | | | | | | |
| | | and in-hospital, | | | | | | | | |
| | | pre-treatment | | | | | | | | |
| | | periods). | | | | | | | | |
| Moderate-to- | Pre- | 21 days prior to | No | — | Yes | Dichotomous | 139 (52.5%) | 135 (50.9%) | 96 (51.6%) | 93 (50.0%) |
| severe COVID-19 | admission | hospitalization | | | | | | | | |
| signs/symptoms | COVID-19 | | | | | | | | | |
| recorded pre- | onset and | | | | | | | | | |
| admission | utilization | | | | | | | | | |
| (inclusive) | | | | | | | | | | |
| Any emergency | Pre- | 21 days prior to | No | — | Yes | Dichotomous | 93 (35.1%) | 96 (36.2%) | 68 (36.6%) | 66 (35.5%) |
| department or | admission | hospitalization | | | | | | | | |
| inpatient | COVID-19 | | | | | | | | | |
| encounter in pre- | onset and | | | | | | | | | |
| admission period | utilization | | | | | | | | | |
| (exclusive) | | | | | | | | | | |
| Use of any | Pre- | 21 days prior to | No | — | Yes | Dichotomous | 27 (10.2%) | 36 (13.6%) | 19 (10.2%) | 25 (13.4%) |
| experimental | admission | hospitalization to | | | | | | | | |
| COVID-19 | COVID-19 | date of treatment | | | | | | | | |
| therapy (HCQ, | onset and | initiation (includes | | | | | | | | |
| Remdesivir, IL- | utilization | both pre-admission | | | | | | | | |
| 6/23, etc) in pre- | | and in-hospital, | | | | | | | | |
| admission or pre- | | pre-treatment | | | | | | | | |
| treatment | | periods). | | | | | | | | |
| periods* | | | | | | | | | | |
| Urban hospital | Hospital | days 0-1 of | No | — | Yes | Dichotomous | 227 (85.7%) | 249 (94.0%) | 172 (92.5%) | 173 (93.0%) |
| setting | facility & | hospitalization | | | | | | | | |
| | admitting | | | | | | | | | |
| | characteristics | | | | | | | | | |
| Teaching | Hospital | days 0-1 of | No | — | Yes | Dichotomous | 158 (59.6%) | 143 (54.0%) | 103 (55.4%) | 109 (58.6%) |
| hospital | facility & | hospitalization | | | | | | | | |
| | admitting | | | | | | | | | |
| | characteristics | | | | | | | | | |
| Hospital with | Hospital | days 0-1 of | No | — | Yes | Dichotomous | 180 (67.9%) | 145 (54.7%) | 112 (60.2%) | 116 (62.4%) |
| 300+ beds | facility & | hospitalization | | | | | | | | |
| | admitting | | | | | | | | | |
| | characteristics | | | | | | | | | |
| Transfer from | Hospital | days 0-1 of | No | — | Yes | Dichotomous | 48 (18.1%) | 47 (17.7%) | 33 (17.7%) | 32 (17.2%) |
| SNF/hospital | facility & | hospitalization | | | | | | | | |
| | admitting | | | | | | | | | |
| | characteristics | | | | | | | | | |
| Emergency | Hospital | days 0-1 of | No | — | Yes | Dichotomous | 179 (67.5%) | 179 (67.5%) | 127 (68.3%) | 131 (70.4%) |
| department or | facility & | hospitalization | | | | | | | | |
| ambulance | admitting | | | | | | | | | |
| encounter on day | characteristics | | | | | | | | | |
| of admission | | | | | | | | | | |
| Emergency or | Hospital | days 0-1 of | No | — | Yes | Dichotomous | 220 (83.0%) | 217 (81.9%) | 153 (82.3%) | 152 (81.7%) |
| trauma admitting | facility & | hospitalization | | | | | | | | |
| type | admitting | | | | | | | | | |
| | characteristics | | | | | | | | | |
| Admitting | Hospital | days 0-1 of | No | — | Yes | Dichotomous | 32 (12.1%) | 28 (10.6%) | 21 (11.3%) | 22 (11.8%) |
| diagnosis for | facility & | hospitalization | | | | | | | | |
| delirium or other | admitting | | | | | | | | | |
| altered mental | characteristics | | | | | | | | | |
| status | | | | | | | | | | |
| No. of days since | Pre- | hospital admission | Yes | Direct (1:1) matching on time from | Yes | Continuous | | | | |
| hospital | treatment | date to the date of | | documented COVID 19 infection to | | numeric | | | | |
| admission | characteristics | treatment initiation | | treatment initiation, no. days | | variable | | | | |
| | | | | categories (0-1, 2-3, 4-5, 6-9, 10-14, | | | | | | |
| | | | | 15-19, 20+) | | | | | | |
| . . . mean (sd) | Pre- | hospital admission | — | — | — | — | 3.07 (1.86) | 3.19 (1.81) | 3.09 (1.91) | 3.14 (1.73) |
| | treatment | date to the date of | | | | | | | | |
| | characteristics | treatment initiation | | | | | | | | |
| . . . median [IQR] | Pre- | hospital admission | — | — | — | — | 2 [2, 3] | 3 [2, 3] | 2 [2, 3] | 3 [2, 3] |
| | treatment | date to the date of | | | | | | | | |
| | characteristics | treatment initiation | | | | | | | | |
| Use of any | Pre- | hospital admission | No | — | Yes | Dichotomous | 157 (59.2%) | 173 (65.3%) | 119 (64.0%) | 124 (66.7%) |
| antibiotic | treatment | date to the date of | | | | | | | | |
| | characteristics | treatment initiation | | | | | | | | |
| On supplemental | Pre- | hospital admission | Yes | Direct (1:1) matching on highest level | Yes | Dichotomous, | 20 (7.5%) | 19 (7.2%) | 11 (5.9%) | 18 (9.7%) |
| oxygen at | treatment | date to the date of | | of respiratory support in 2 days pre- | | oxygen | | | | |
| treatment | characteristics | treatment initiation | | treatment (inclusive), no oxygen vs | | status at | | | | |
| | | | | supplementary oxygen. Note this RSS | | treatment | | | | |
| | | | | matching criteria uses a 2 day | | index date | | | | |
| | | | | lookback window, whereas the PS | | | | | | |
| | | | | inputs and results shown in columns | | | | | | |
| | | | | H-K assess oxygen status on the | | | | | | |
| | | | | treatment index date only. | | | | | | |
| In ICU at | Pre- | hospital admission | No | — | Yes | Dichotomous | 54 (20.4%) | 60 (22.6%) | 38 (20.4%) | 42 (22.6%) |
| treatment | treatment | date to the date of | | | | | | | | |
| | characteristics | treatment initiation | | | | | | | | |
| No. unique | Pre- | hospital admission | No | — | Yes | Continuous | | | | |
| department codes | treatment | date to the date of | | | | numeric | | | | |
| observed | characteristics | treatment initiation | | | | variable | | | | |
| . . . mean (sd) | Pre- | hospital admission | — | — | — | — | 12.46 (4.92) | 12.93 (4.95) | 12.43 (5.10) | 12.73 (4.96) |
| | treatment | date to the date of | | | | | | | | |
| | characteristics | treatment initiation | | | | | | | | |
| . . . median [IQR] | Pre- | hospital admission | — | — | — | — | 12 [9, 15.50] | 13 [9, 16] | 12 [9, 16] | 12.50 [9, 16] |
| | treatment | date to the date of | | | | | | | | |
| | characteristics | treatment initiation | | | | | | | | |

TABLE 7G
AP COHORT BALANCE

| | | Absolute |
|---|---|---|
| | Absolute | Standard |
| | Standard | Difference |
| | Difference | (RSS |
| | (RSS | and PS |
| Variable | only) | matched) |
| Month of treatment initiation | 0.016 | 0.083 |
| Age | 0.006 | 0.020 |
| Gender | 0.000 | 0.000 |
| U.S. Region | 0.191 | 0.014 |
| No. of medical encounters | 0.084 | 0.126 |
| No. of unique medications dispensed | 0.244 | 0.064 |
| Charlson Comorbidity Index | 0.026 | 0.141 |
| Cancer | 0.017 | 0.023 |
| Chronic pulmonary disease | 0.177 | 0.014 |
| Cardiovascular disease (any) | 0.091 | 0.086 |
| Arrhythmia | 0.103 | 0.092 |
| Hypertension | 0.122 | 0.043 |
| Dementia | 0.018 | 0.081 |
| Diabetes | 0.017 | 0.102 |
| Tobacco use recorded | 0.000 | 0.016 |
| Kidney or liver disease | 0.037 | 0.091 |
| Immunosuppressive condition | 0.022 | 0.108 |
| Overweight or obese | 0.062 | 0.017 |
| Use of any antithrombotic therapy (anticoags, | 0.155 | 0.025 |
| antiplatelets, antifibrinolytics) | | |
| Use of statin medication | 0.242 | 0.028 |
| Use of any steroid medication | 0.053 | 0.025 |
| Moderate-to-severe COVID-19 signs/symptoms | 0.030 | 0.032 |
| recorded pre-admission (inclusive) | | |
| Any emergency department or inpatient | 0.024 | 0.022 |
| encounter in pre-admission period (exclusive) | | |
| Use of any experimental COVID-19 therapy | 0.105 | 0.100 |
| (HCQ, Remdesivir, IL-6/23, etc) in pre- | | |
| admission or pre-treatment periods* | | |
| Urban hospital setting | 0.277 | 0.021 |
| Teaching hospital | 0.114 | 0.065 |
| Hospital with 300+ beds | 0.274 | 0.044 |
| Transfer from SNF or hospital | 0.010 | 0.014 |
| Emergency department or ambulance encounter | 0.000 | 0.047 |
| on day of admission | | |
| Emergency or trauma admitting type | 0.030 | 0.014 |
| Admitting diagnosis for delirium or other altered | 0.048 | 0.017 |
| mental status | | |
| No. of days since hospital admission | 0.064 | 0.027 |
| Use of any antibiotic in-hospital | 0.125 | 0.057 |
| Supplemental oxygen use at treatment | 0.014 | 0.141 |
| In ICU at treatment | 0.055 | 0.052 |
| No. unique department codes observed in- | 0.095 | 0.060 |
| hospital | | |
| Average standardized absolute mean difference | 0.082 | 0.053 |

TABLE 7H
AP OUTCOMES

| | RSS only | RSS only | RSS and PS | RSS and PS |
|---|---|---|---|---|
| | typical | atypical | typical | atypical |
| | anti- | anti- | anti- | anti- |
| | psychotic | psychotic | psychotic | psychotic |
| Cohort | cohort | cohort | cohort | cohort |
| Treatment | typical | atypical | typical | atypical |
| | anti- | anti- | anti- | anti- |
| | psychotic | psychotic | psychotic | psychotic |
| Treatment | Experi- | Referent | Experi- | Referent |
| classification | mental | | mental | |
| Matching criteria | RSS only | RSS only | RSS and PS | RSS and PS |
| Number of patients | 265 | 265 | 186 | 186 |
| Number of | 19 | 32 | 13 | 26 |
| patients requiring | | | | |
| mechanical | | | | |
| ventilation | | | | |
| Risk of | 71.7 | 120.75 | 69.89 | 139.78 |
| mechanical | | | | |
| ventilation | | | | |
| per 1000 patients | | | | |
| Risk ratio vs | 0.59 | Referent | 0.5 | Referent |
| referent of | | | | |
| mechanical | | | | |
| ventilation | | | | |
| 95% confidence | 0.35 | Referent | 0.27 | Referent |
| interval of risk | | | | |
| ratio vs referent | | | | |
| of mechanical | | | | |
| ventilation, | | | | |
| lower bound | | | | |
| 95% confidence | 1.02 | Referent | 0.94 | Referent |
| interval of risk | | | | |
| ratio vs referent | | | | |
| of mechanical | | | | |
| ventilation, | | | | |
| upper bound | | | | |
| Odds ratio | 0.56 | Referent | 0.46 | Referent |
| of mechanical | | | | |
| ventilation | | | | |
| versus referent | | | | |
| 95% confidence i | 0.31 | Referent | 0.23 | Referent |
| nterval of | | | | |
| odds ratio | | | | |
| of mechanical | | | | |
| ventilation | | | | |
| versus referent, | | | | |
| lower bound | | | | |
| 95% confidence | 1.02 | Referent | 0.93 | Referent |
| interval of | | | | |
| odds ratio | | | | |
| of mechanical | | | | |
| ventilation | | | | |
| versus referent, | | | | |
| upper bound | | | | |
| p-value of | 0.058 | Referent | 0.031 | Referent |
| odds ratio of | | | | |
| mechanical | | | | |
| ventilation versus | | | | |
| referent | | | | |

TABLE 7I
DRUG LIST

| | | | Experimental | |
|---|---|---|---|---|
| | | | or | |
| Drug | Comparison | Class | Comparator | Notes |
| Indomethacin | NSAIDS | NSAID | experimental | |
| celecoxib | NSAIDS | NSAID | comparator | |
| haloperidol | antipsychotics | typical | experimental | |
| chlorpromazine | antipsychotics | typical | experimental | |
| fluphenazine | antipsychotics | typical | experimental | |
| aripiprazole | antipsychotics | atypical | comparator | |
| olanzapine | antipsychotics | atypical | comparator | |
| quetiapine | antipsychotics | atypical | comparator | |
| risperidone | antipsychotics | atypical | comparator | |
| brexpiprazole | antipsychotics | atypical | comparator | |
| paliperidone | antipsychotics | atypical | comparator | |

Observation of Mechanical Ventilation Outcomes in Inpatient New Users of Typical Antipsychotics (Treatment Arm) Vs. Atypical Antipsychotics (Active Comparator) Using Real-World Data

An incident user, active comparator design (W. A. Ray, Evaluating medication effects outside of clinical trials: new-user designs. Am. J Epidemiol. 158, 915-920 (2003); S. Schneeweiss, A basic study design for expedited safety signal evaluation based on electronic healthcare data. Pharmacoepidemiol. Drug Saf 19, 858-868 (2010)) was used to assess the risk of mechanical ventilation among hospitalized COVID-19 patients treated with typical or atypical antipsychotics in an inpatient setting. See Table 7A-I for a list of drugs included in each category. To permit assessment of day-level in-hospital confounders and outcomes, this analysis was restricted to hospitalized patients observable in hospital chargemaster data. Prevalent users of typical or atypical antipsychotics (any prescription fill or chargemaster-documented use in 60 days prior) and patients with evidence of mechanical ventilation in the 21 days prior to and including the date of treatment initiation were excluded from this analysis.

Using RSS, hospitalized patients treated with typical antipsychotics were matched at a 1:1 ratio to controls randomly selected among patients treated with atypical antipsychotics, with direct matching (1:1 fixed ratio) on calendar date of treatment (±7 days), age (±5 years), sex, Charlson comorbidity index (exact) (H. Quan, et al., Coding algorithms for defining comorbidities in ICD-9-CM and ICD-10 administrative data. Med. Care. 43, 1130-1139 (2005)), time since hospital admission, and disease severity as defined with a simplified version of the World Health Organization's ordinal scale for clinical improvement (WHO R&D Blueprint novel Coronavirus: COVID-19 Therapeutic Trial Synopsis. World Health Organization, 2020, (available at https://www.who.int/blueprint/priority-diseases/key-action/COVID-19_Treatment_Trial_Design_Master_Protocol_synopsis_Final_18022020.pdf)). This risk set sampled population was further matched on a PS estimated using logistic regression with 36 demographic and clinical risk factors, including covariates related to baseline medical history, admitting status, and disease severity at treatment. Balance between typical and atypical treatment groups was evaluated by comparison of absolute standardized differences in covariates, with an absolute standardized difference of less than 0.2 indicating good balance between the treatment groups (P. C. Austin, Balance diagnostics for comparing the distribution of baseline covariates between treatment groups in propensity-score matched samples. Stat. Med. 28, 3083-3107 (2009)).

The primary analysis was an intention-to-treat design, with follow-up beginning 1 day after the date of typical or atypical antipsychotic treatment initiation, and ending on the earliest occurrence of 30 days of follow-up reached, discharge from hospital, or end of patient data. Odds ratios for the primary outcome of inpatient mechanical ventilation were estimated for the RSS+PS matched population as well as for the RSS matched population.

### Results

Conserved Coronavirus Proteins Often Retain the Same Cellular Localization

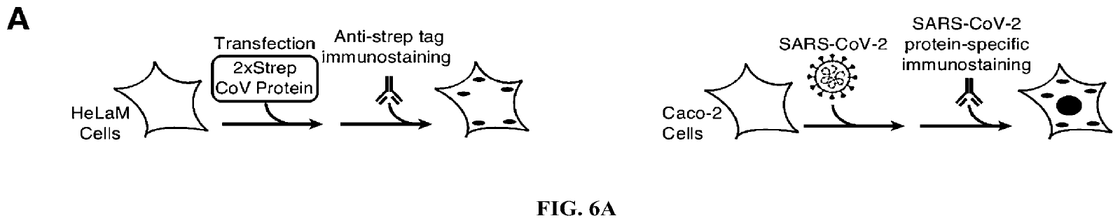

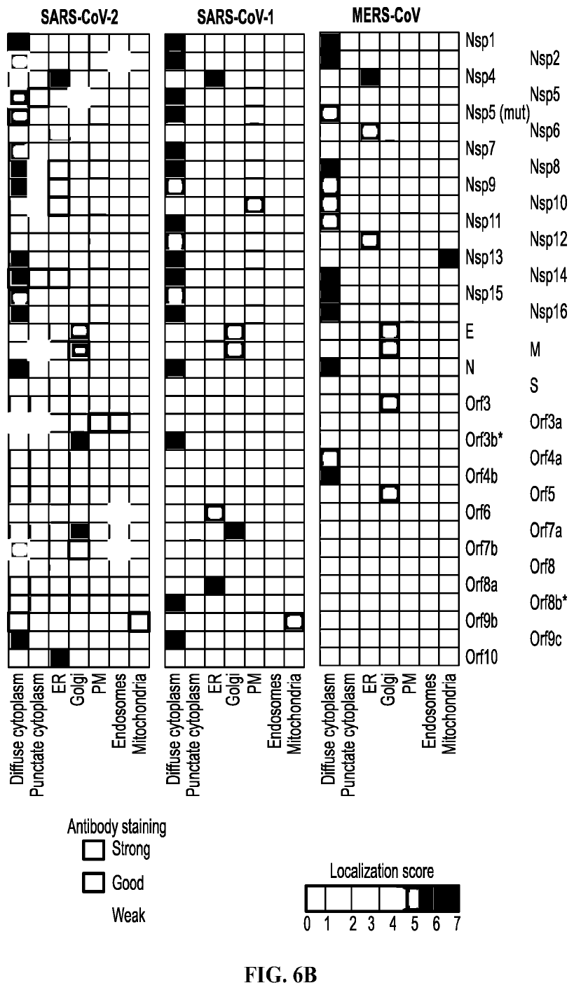

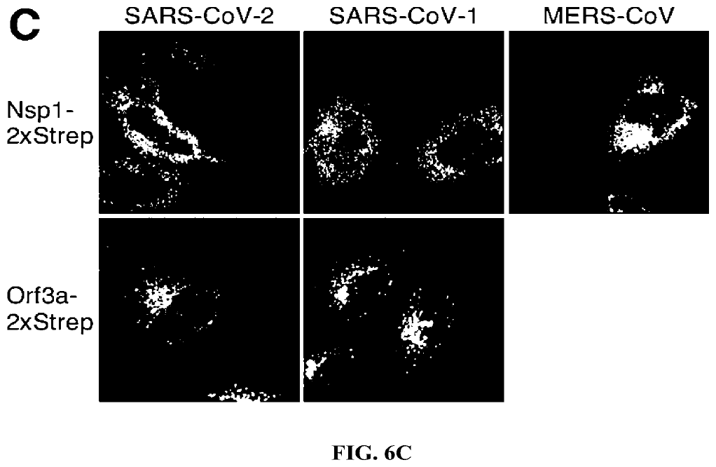

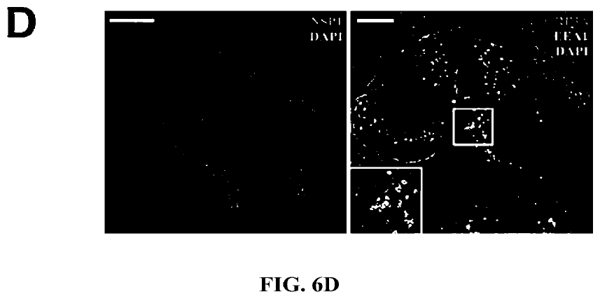

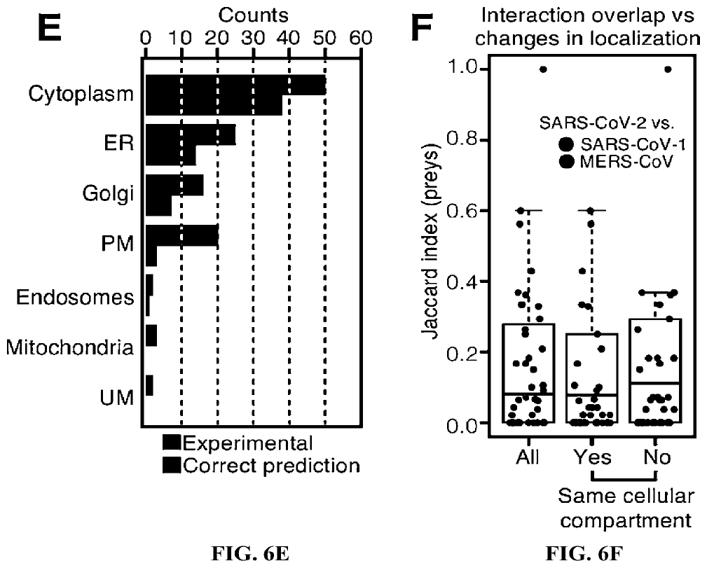

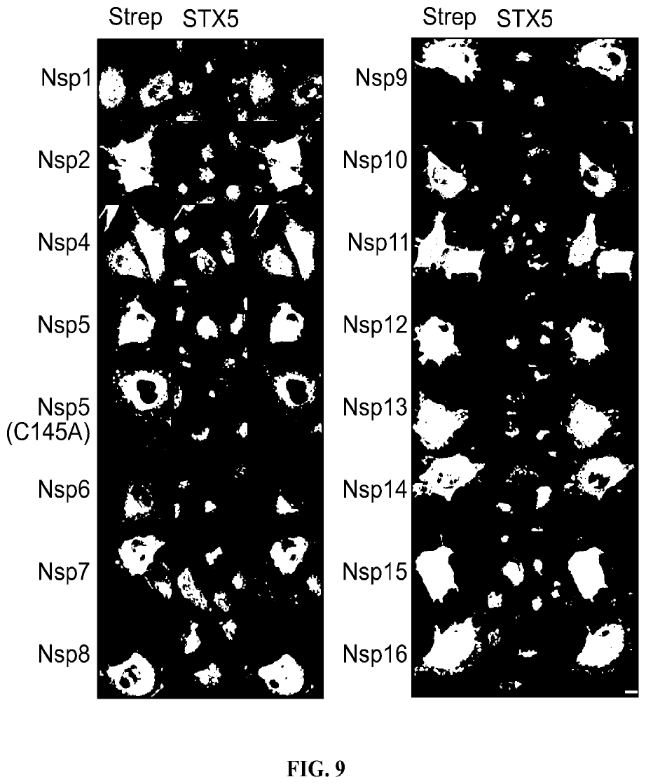

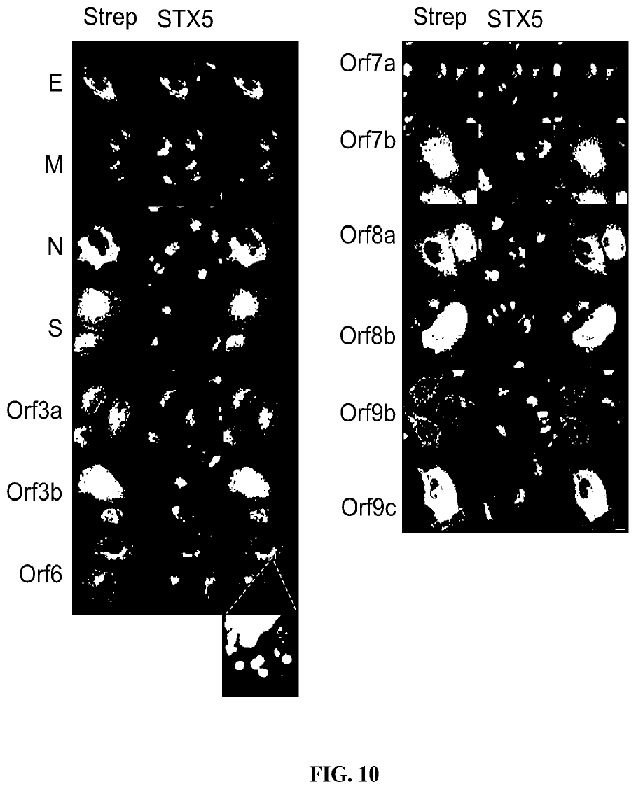

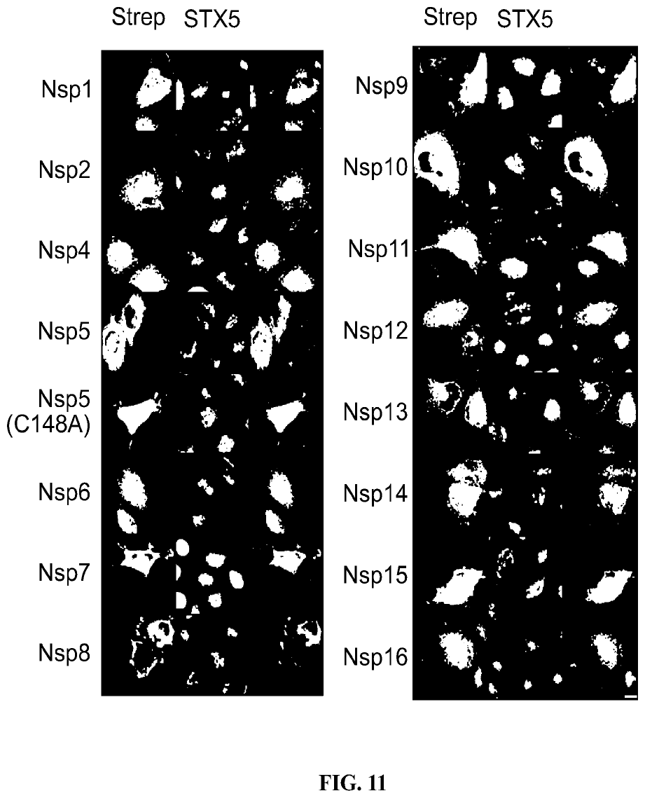

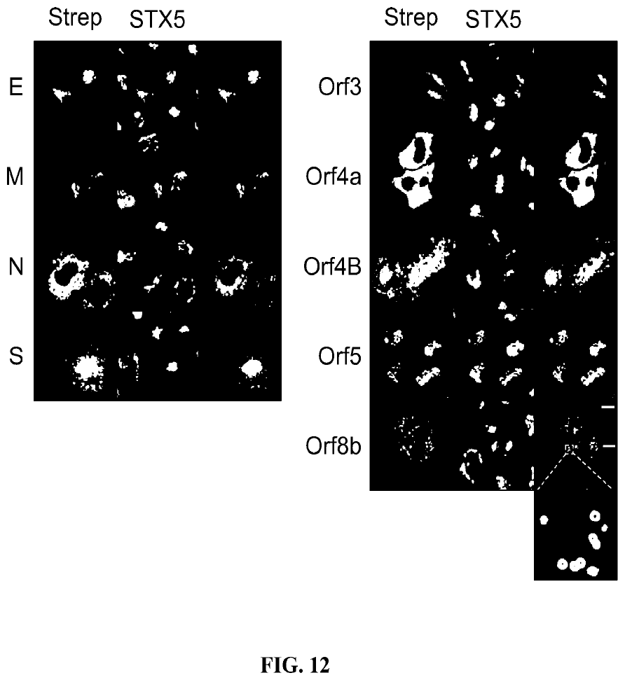

As protein localization can provide important information regarding function, the cellular localization of individually expressed coronavirus proteins was assessed, in addition to mapping their interactions (FIG. 6A) Immunofluorescence localization analysis of all 2×Strep-tagged SARS-CoV-2, SARS-CoV-1, and MERS-CoV proteins highlights similar patterns of localization for the vast majority of shared protein homologs in HelaM cells (FIG. 6B). This supports the hypothesis that conserved proteins share functional similarities. A notable exception is Nsp13, which appears to localize to the cytoplasm for SARS-CoV-2 and SARS-CoV-1; however, MERS-CoV Nsp13 appears to localize to the mitochondria (FIG. 6B and FIG. 7-12 and Table 8 Å-D). To assess the localization of SARS-CoV-2 proteins in the context of infected cells, antibodies against SARS-CoV-2 proteins were raised and validated with the individually-expressed 2×Strep-tagged proteins. Using the 14 antibodies with confirmed specificity, it was observed that localization of viral proteins in infected Caco-2 cells sometimes differed from their localization when expressed individually (FIG. 6B and FIG. 13 and Table 8 Å-D). This likely results from recruitment of viral proteins and complexes into replication compartments, as well as remodeling of the secretory pathway during viral infection. For proteins such as Nsp1 and Orf3a, which are not known to be involved in viral replication, their localization is consistent both when expressed individually and in the context of viral infection (FIG. 6C and FIG. 6D).

Referring to FIG. 6A, an overview of experimental design to determine localization of Strep-tagged SARS-CoV-2, SARS-CoV-1, and MERS-CoV proteins in HeLaM cells (left) or of viral proteins upon SARS-CoV-2 infection in Caco-2 cells (right) is shown.

Referring to FIG. 6B, relative localization for all coronavirus proteins across viruses expressed individually (blue color bar; * indicates viral proteins of high sequence divergence) or in SARS-CoV-2 infected cells (colored box outlines) is shown.

Referring to FIG. 6C and FIG. 6D, the localization of Nsp1 and Orf3a expressed individually (FIG. 6C) or during infection (FIG. 6D) for representative images of all tagged constructs and viral proteins imaged during infection are shown. See FIG. 7-13, respectively. Scale bars=10 μm.

TABLE 8A
LOCALIZATION EXP REPORTER

| | Viral | Diffuse | Punctate | | | | | | |
|---|---|---|---|---|---|---|---|---|---|
| Virus | Protein | cytoplasm | cytoplasmic | ER | Golgi | PM | Endosomes | Mitochondria | Notes |
| SARS_CoV_2 | NSP1 | 6 | | | | 1 | | | Construct |
| | | | | | | | | | is |
| | | | | | | | | | expressed |
| | | | | | | | | | at very low |
| | | | | | | | | | levels. |
| SARS_CoV_2 | NSP2 | 4 | | | | 3 | | | Some |
| | | | | | | | | | enrichment |
| | | | | | | | | | at |
| | | | | | | | | | lamellipodia. |
| SARS_CoV_2 | NSP4 | | | 7 | | | | | |
| SARS_CoV_2 | NSP5 (wt) | 5 | | | | 2 | | | Some |
| | | | | | | | | | enrichment |
| | | | | | | | | | at |
| | | | | | | | | | lamellipodia. |
| SARS_CoV_2 | NSP5_C148A | 5 | | | | 2 | | | Some |
| | | | | | | | | | enrichment |
| | | | | | | | | | at |
| | | | | | | | | | lamellipodia. |
| SARS_CoV_2 | NSP6 | | | 4 | 3 | | | | |
| SARS_CoV_2 | NSP7 | 5 | | | | 2 | | | Some |
| | | | | | | | | | enrichment |
| | | | | | | | | | at |
| | | | | | | | | | lamellipodia. |
| SARS_CoV_2 | NSP8 | 6 | | | | 1 | | | Some |
| | | | | | | | | | enrichment |
| | | | | | | | | | at |
| | | | | | | | | | lamellipodia. |
| SARS_CoV_2 | NSP9 | 7 | | | | | | | Some |
| | | | | | | | | | enrichment |
| | | | | | | | | | at |
| | | | | | | | | | lamellipodia. |
| SARS_CoV_2 | NSP10 | 4 | | | | 3 | | | Strong |
| | | | | | | | | | enrichment |
| | | | | | | | | | at surface |
| | | | | | | | | | when |
| | | | | | | | | | expressed |
| | | | | | | | | | at high |
| | | | | | | | | | levels. |
| SARS_CoV_2 | NSP11 | 4 | | | | 3 | | | Some |
| | | | | | | | | | enrichment |
| | | | | | | | | | at |
| | | | | | | | | | lamellipodia. |
| SARS_CoV_2 | NSP12 | 3 | | | | 4 | | | Some |
| | | | | | | | | | enrichment |
| | | | | | | | | | at |
| | | | | | | | | | lamellipodia. |
| SARS_CoV_2 | NSP13 | 6 | | | | 1 | | | Some |
| | | | | | | | | | enrichment |
| | | | | | | | | | at |
| | | | | | | | | | lamellipodia. |
| SARS_CoV_2 | NSP14 | 6 | | | | 1 | | | Some |
| | | | | | | | | | enrichment |
| | | | | | | | | | at |
| | | | | | | | | | lamellipodia. |
| SARS_CoV_2 | NSP15 | 5 | | | | 2 | | | Some |
| | | | | | | | | | enrichment |
| | | | | | | | | | at |
| | | | | | | | | | lamellipodia. |
| SARS_CoV_2 | NSP16 | 6 | | | | 1 | | | Some |
| | | | | | | | | | enrichment |
| | | | | | | | | | at |
| | | | | | | | | | lamellipodia. |
| SARS_CoV_2 | Orf3A | | | 1 | 1 | 1 | 4 | | Levels at |
| | | | | | | | | | surface |
| | | | | | | | | | increase |
| | | | | | | | | | with |
| | | | | | | | | | expression. |
| | | | | | | | | | At very |
| | | | | | | | | | low levels |
| | | | | | | | | | see puncta |
| | | | | | | | | | which |
| | | | | | | | | | most likely |
| | | | | | | | | | localise to |
| | | | | | | | | | nuclear |
| | | | | | | | | | envelope |
| SARS_CoV_2 | Orf3B | | | | 7 | | | | Only a |
| | | | | | | | | | very small |
| | | | | | | | | | number of |
| | | | | | | | | | cells |
| | | | | | | | | | showing |
| | | | | | | | | | expression. |
| SARS_CoV_2 | Orf6 | | 2 | 1 | 4 | | | | Predominantly |
| | | | | | | | | | Golgi |
| | | | | | | | | | staining |
| | | | | | | | | | with small |
| | | | | | | | | | puncta |
| | | | | | | | | | most likely |
| | | | | | | | | | associated |
| | | | | | | | | | with the |
| | | | | | | | | | ER. |
| SARS_CoV_2 | Orf7A | | 1 | | 6 | | | | Lots of |
| | | | | | | | | | small |
| | | | | | | | | | membrane |
| | | | | | | | | | bound |
| | | | | | | | | | puncta in |
| | | | | | | | | | addition to |
| | | | | | | | | | Golgi |
| | | | | | | | | | staining. |
| SARS_CoV_2 | Orf7B | 4 | | 2 | | 1 | | | At low |
| | | | | | | | | | levels in |
| | | | | | | | | | the ER. As |
| | | | | | | | | | expression |
| | | | | | | | | | increases |
| | | | | | | | | | becomes |
| | | | | | | | | | more |
| | | | | | | | | | cytoplasmic. |
| SARS_CoV_2 | Orf8 | | | 4 | 3 | | | | Some |
| | | | | | | | | | nuclear |
| | | | | | | | | | envelope |
| | | | | | | | | | staining. |
| SARS_CoV_2 | Orf9B | 2 | | | | | | 5 | Cytoplasmic |
| | | | | | | | | | localisation |
| | | | | | | | | | increases |
| | | | | | | | | | with |
| | | | | | | | | | expression. |
| SARS_CoV_2 | Orf9C | 7 | | | | | | | |
| SARS_CoV_2 | Orf10 | | | 7 | | | | | Some |
| | | | | | | | | | nuclear |
| | | | | | | | | | envelope |
| | | | | | | | | | localisation |
| SARS_CoV_2 | M | | | 2 | 5 | | | | At high |
| | | | | | | | | | levels |
| | | | | | | | | | observe |
| | | | | | | | | | protein at |
| | | | | | | | | | PM and |
| | | | | | | | | | tubular |
| | | | | | | | | | structures |
| | | | | | | | | | emanating |
| | | | | | | | | | from ER |
| | | | | | | | | | and Golgi. |
| SARS_CoV_2 | E | | | 2 | 5 | | | | ER |
| | | | | | | | | | localisation |
| | | | | | | | | | increases |
| | | | | | | | | | with |
| | | | | | | | | | expression. |
| SARS_CoV_2 | N | 6 | | | | 1 | | | Some |
| | | | | | | | | | enrichment |
| | | | | | | | | | at |
| | | | | | | | | | lamellipodia. |
| SARS_CoV_2 | S | | | 2 | 1 | 4 | | | |
| SARS_CoV_1 | NSP1 | 6 | | | | 1 | | | Construct |
| | | | | | | | | | is |
| | | | | | | | | | expressed |
| | | | | | | | | | at very low |
| | | | | | | | | | levels. |
| SARS_CoV_1 | NSP2 | 6 | | | | 1 | | | Some |
| | | | | | | | | | enrichment |
| | | | | | | | | | at |
| | | | | | | | | | lamellipodia. |
| SARS_CoV_1 | NSP3 | | | | | | | | Not |
| | | | | | | | | | determined. |
| SARS_CoV_1 | NSP4 | | | 7 | | | | | |
| SARS_CoV_1 | NSP5 (wt) | 6 | | | | 1 | | | Some |
| | | | | | | | | | enrichment |
| | | | | | | | | | at |
| | | | | | | | | | lamellipodia. |
| SARS_CoV_1 | NSP5_C148A | 6 | | | | 1 | | | Some |
| | | | | | | | | | enrichment |
| | | | | | | | | | at |
| | | | | | | | | | lamellipodia. |
| SARS_CoV_1 | NSP6 | | | 4 | 3 | | | | |
| SARS_CoV_1 | NSP7 | 6 | | | | 1 | | | Some |
| | | | | | | | | | enrichment |
| | | | | | | | | | at |
| | | | | | | | | | lamellipodia. |
| SARS_CoV_1 | NSP8 | 6 | | | | 1 | | | Some |
| | | | | | | | | | enrichment |
| | | | | | | | | | at |
| | | | | | | | | | lamellipodia. |
| SARS_CoV_1 | NSP9 | 5 | | | | 2 | | | Some |
| | | | | | | | | | enrichment |
| | | | | | | | | | at |
| | | | | | | | | | lamellipodia. |
| SARS_CoV_1 | NSP10 | 2 | | | | 5 | | | Strong |
| | | | | | | | | | enrichment |
| | | | | | | | | | at surface |
| | | | | | | | | | when |
| | | | | | | | | | expressed |
| | | | | | | | | | at high |
| | | | | | | | | | levels. |
| SARS_CoV_1 | NSP11 | 6 | | | | 1 | | | Some |
| | | | | | | | | | enrichment |
| | | | | | | | | | at |
| | | | | | | | | | lamellipodia. |
| SARS_CoV_1 | NSP12 | 5 | | | | 2 | | | Some |
| | | | | | | | | | enrichment |
| | | | | | | | | | at |
| | | | | | | | | | lamellipodia. |
| SARS_CoV_1 | NSP13 | 6 | | | | 1 | | | Some |
| | | | | | | | | | enrichment |
| | | | | | | | | | at |
| | | | | | | | | | lamellipodia. |
| SARS_CoV_1 | NSP14 | 6 | | | | 1 | | | Some |
| | | | | | | | | | enrichment |
| | | | | | | | | | at |
| | | | | | | | | | lamellipodia. |
| SARS_CoV_1 | NSP15 | 5 | | | | 2 | | | Some |
| | | | | | | | | | enrichment |
| | | | | | | | | | at |
| | | | | | | | | | lamellipodia. |
| SARS_CoV_1 | NSP16 | 6 | | | | 1 | | | Some |
| | | | | | | | | | enrichment |
| | | | | | | | | | at |
| | | | | | | | | | lamellipodia. |
| SARS_CoV_1 | Orf3A | | | 1 | 1 | 1 | 4 | | Levels at |
| | | | | | | | | | surface |
| | | | | | | | | | increase |
| | | | | | | | | | with |
| | | | | | | | | | expression. |
| | | | | | | | | | At very |
| | | | | | | | | | low levels |
| | | | | | | | | | see puncta |
| | | | | | | | | | which |
| | | | | | | | | | localise to |
| | | | | | | | | | nuclear |
| SARS_CoV_1 | Orf3B | 7 | | | | | | | Only a |
| | | | | | | | | | very small |
| | | | | | | | | | number of |
| | | | | | | | | | cells |
| | | | | | | | | | showing |
| | | | | | | | | | expression. |
| | | | | | | | | | Some |
| | | | | | | | | | nuclear |
| | | | | | | | | | staining in |
| | | | | | | | | | addition to |
| | | | | | | | | | cytoplasmic |
| | | | | | | | | | staining. |
| SARS_CoV_1 | Orf6 | | 1 | 5 | 1 | | | | Doughnut |
| | | | | | | | | | or ring like |
| | | | | | | | | | structure |
| | | | | | | | | | associated |
| | | | | | | | | | with ER. |
| SARS_CoV_1 | Orf7A | | 1 | | 6 | | | | Lots of |
| | | | | | | | | | small |
| | | | | | | | | | membrane |
| | | | | | | | | | bound |
| | | | | | | | | | puncta in |
| | | | | | | | | | addition to |
| | | | | | | | | | Golgi |
| | | | | | | | | | staining. |
| SARS_CoV_1 | Orf7B | 3 | | 2 | 1 | 1 | | | |
| SARS_CoV_1 | Orf8A | | | 7 | | | | | Nuclear |
| | | | | | | | | | envelope |
| | | | | | | | | | staining. |
| SARS_CoV_1 | Orf8B | 6 | | | | 1 | | | |
| SARS_CoV_1 | Orf9B | 2 | | | | | | 5 | Cytoplasmic |
| | | | | | | | | | localisation |
| | | | | | | | | | increases |
| | | | | | | | | | with |
| | | | | | | | | | expression. |
| SARS_CoV_1 | Orf9C | 7 | | | | | | | |
| SARS_CoV_1 | M | | | 2 | 5 | | | | At high |
| | | | | | | | | | levels |
| | | | | | | | | | observe |
| | | | | | | | | | protein at |
| | | | | | | | | | PM and |
| | | | | | | | | | tubular |
| | | | | | | | | | structures |
| | | | | | | | | | emanating |
| | | | | | | | | | from ER |
| | | | | | | | | | and Golgi. |
| SARS_CoV_1 | E | | | 2 | 5 | | | | ER |
| | | | | | | | | | localisation |
| | | | | | | | | | increases |
| | | | | | | | | | with |
| | | | | | | | | | expression. |
| SARS_CoV_1 | N | 6 | | | | 1 | | | Some |
| | | | | | | | | | enrichment |
| | | | | | | | | | at |
| | | | | | | | | | lamellipodia. |
| SARS_CoV_1 | S | | | 2 | 1 | 4 | | | |
| MERS | NSP1 | 7 | | | | | | | Construct |
| | | | | | | | | | is |
| | | | | | | | | | expressed |
| | | | | | | | | | at very low |
| | | | | | | | | | levels. |
| MERS | NSP2 | 6 | | | | 1 | | | Some |
| | | | | | | | | | enrichment |
| | | | | | | | | | at |
| | | | | | | | | | lamellipodia. |
| MERS | NSP3 (wt) | | | 7 | | | | | |
| MERS | NSP3_C740A | | | 7 | | | | | |
| MERS | NSP4 | | | 7 | | | | | Present on |
| | | | | | | | | | nuclear |
| | | | | | | | | | envelop at |
| | | | | | | | | | high |
| | | | | | | | | | expression |
| | | | | | | | | | levels |
| MERS | NSP5 (wt) | 3 | | | | 4 | | | Some |
| | | | | | | | | | enrichment |
| | | | | | | | | | at |
| | | | | | | | | | lamellipodia. |
| MERS | NSP5_C148A | 5 | | | | 2 | | | Some |
| | | | | | | | | | enrichment |
| | | | | | | | | | at |
| | | | | | | | | | lamellipodia. |
| MERS | NSP6 | | | 5 | 2 | | | | |
| MERS | NSP7 | 4 | | | | 3 | | | Some |
| | | | | | | | | | enrichment |
| | | | | | | | | | at |
| | | | | | | | | | lamellipodia. |
| MERS | NSP8 | 6 | | | | 1 | | | Expressed |
| | | | | | | | | | at very |
| | | | | | | | | | high |
| | | | | | | | | | levels. |
| MERS | NSP9 | 5 | | | | 2 | | | Some |
| | | | | | | | | | enrichment |
| | | | | | | | | | at |
| | | | | | | | | | lamellipodia. |
| MERS | NSP10 | 5 | | | | 2 | | | Strong |
| | | | | | | | | | enrichment |
| | | | | | | | | | at surface |
| | | | | | | | | | when |
| | | | | | | | | | expressed |
| | | | | | | | | | at high |
| | | | | | | | | | levels. |
| MERS | NSP11 | 5 | | | | 2 | | | Some |
| | | | | | | | | | enrichment |
| | | | | | | | | | at |
| | | | | | | | | | lamellipodia. |
| MERS | NSP12 | 2 | | 5 | | | | | Some cells |
| | | | | | | | | | mainly |
| | | | | | | | | | show |
| | | | | | | | | | cytoplasmic |
| | | | | | | | | | staining |
| | | | | | | | | | and others |
| | | | | | | | | | ER. |
| MERS | NSP13 | | | | | 1 | | 6 | Some |
| | | | | | | | | | enrichment |
| | | | | | | | | | at |
| | | | | | | | | | lamellipodia. |
| MERS | NSP14 | 6 | | | | 1 | | | Some |
| | | | | | | | | | enrichment |
| | | | | | | | | | at |
| | | | | | | | | | lamellipodia. |
| MERS | NSP15 | 6 | | | | 1 | | | Some |
| | | | | | | | | | enrichment |
| | | | | | | | | | at |
| | | | | | | | | | lamellipodia. |
| MERS | NSP16 | 6 | | | | 1 | | | Some |
| | | | | | | | | | enrichment |
| | | | | | | | | | at |
| | | | | | | | | | lamellipodia. |
| MERS | Orf3 | | | 2 | 5 | | | | At low |
| | | | | | | | | | levels |
| | | | | | | | | | predominantly |
| | | | | | | | | | localised |
| | | | | | | | | | to Golgi. |
| | | | | | | | | | As |
| | | | | | | | | | expression |
| | | | | | | | | | increases |
| | | | | | | | | | more |
| | | | | | | | | | found at |
| | | | | | | | | | ER. |
| MERS | Orf4A | 5 | | | | 2 | | | |
| MERS | Orf4B | 7 | | | | | | | Nuclear |
| | | | | | | | | | staining in |
| | | | | | | | | | small |
| | | | | | | | | | number of |
| | | | | | | | | | cells. |
| MERS | Orf5 | | 1 | 1 | 5 | | | | In addition |
| | | | | | | | | | to Golgi |
| | | | | | | | | | staining |
| | | | | | | | | | there are |
| | | | | | | | | | small |
| | | | | | | | | | puncta |
| | | | | | | | | | found in |
| | | | | | | | | | the |
| | | | | | | | | | cytoplasm |
| | | | | | | | | | possibly |
| | | | | | | | | | associated |
| | | | | | | | | | with ER. |
| MERS | Orf8B | | 3 | 4 | | | | | In addition |
| | | | | | | | | | to ER |
| | | | | | | | | | labelling |
| | | | | | | | | | there are |
| | | | | | | | | | doughnut |
| | | | | | | | | | shaped |
| | | | | | | | | | structures |
| | | | | | | | | | found in |
| | | | | | | | | | the |
| | | | | | | | | | cytoplasm |
| | | | | | | | | | possibly |
| | | | | | | | | | associated |
| | | | | | | | | | with ER. |
| MERS | M | | | 2 | 5 | | | | At high |
| | | | | | | | | | levels |
| | | | | | | | | | observe |
| | | | | | | | | | protein at |
| | | | | | | | | | PM and |
| | | | | | | | | | tubular |
| | | | | | | | | | structures |
| | | | | | | | | | emanating |
| | | | | | | | | | from ER |
| | | | | | | | | | and Golgi. |
| MERS | E | | | 2 | 5 | | | | ER |
| | | | | | | | | | localisation |
| | | | | | | | | | increases |
| | | | | | | | | | with |
| | | | | | | | | | expression. |
| MERS | N | 7 | | | | 1 | | | |
| MERS | S | | | 2 | 1 | 4 | | | |

TABLE 8B
LOCALIZATION EXP ANTIBODY

| | | Diffuse | Punctate | | | | | |
|---|---|---|---|---|---|---|---|---|
| Virus | Viral Protein | cytoplasm | cytoplasmic | ER | Golgi | PM | Endosomes | Mitochondria |
| SARS_CoV_2 | NSP1 | XXX | | | | | | |
| SARS_CoV_2 | NSP2 | X | XXX | | | | X | |
| SARS_CoV_2 | NSP5 | X | XX | | X | | | |
| SARS_CoV_2 | NSP7 | | XXX | X | | | | |
| SARS_CoV_2 | NSP8 | | X | XX | | | | |
| SARS_CoV_2 | NSP9 | | X | XX | | | | |
| SARS_CoV_2 | NSP10 | | X | XX | | | | |
| SARS_CoV_2 | NSP11/12 (did | | | | | | | |
| | NOT work) | | | | | | | |
| SARS_CoV_2 | NSP14 (high | X | X | X | | | | |
| | background), | | | | | | | |
| | difficult to | | | | | | | |
| | judge) | | | | | | | |
| SARS_CoV_2 | NSP16 (did | | | | | | | |
| | NOT work) | | | | | | | |
| SARS_CoV_2 | ORF3A | X | X | | | XXX | XXX | |
| SARS_CoV_2 | ORF6 | X | XX | | | | X | |
| SARS_CoV_2 | ORF7A (did | | | | | | | |
| | NOT work) | | | | | | | |
| SARS_CoV_2 | ORF7B | X | | | XX | | X | |
| SARS_CoV_2 | ORF8 (weak/no | | | | | | | |
| | specific | | | | | | | |
| | staining) | | | | | | | |
| SARS_CoV_2 | ORF9A (B) | XX | | | | | | XXX |
| SARS_CoV_2 | ORF9B (C Did | | | | | | | |
| | not work) | | | | | | | |
| SARS_CoV_2 | M (sheep) | | X (vesicular) | | X | XX | | |
| SARS_CoV_2 | N | XXX | | | | | | |
| SARS_CoV_2 | S (could not do) | | | | | | | |

xxx: strong,
xx: moderate,
x: weak verified with marker

TABLE 8C
LOCALIZATION PREDICTIONS

| | Viral | | | | | | | | Cell | Endoplasmic | | Golgi | Lysosome/ | |
|---|---|---|---|---|---|---|---|---|---|---|---|---|---|---|
| Virus | protein ID | Localisation | Localisation | Type | Nucleus | Cytoplasm | Extracellular | Mitochondrion | membrane | reticulum | Plastid | apparatus | Vacuole | Peroxisome |
| SARS_CoV_2 | nsp1 | Cytoplasm/PM | Cytoplasm | Soluble | 0.1428 | 0.4626 | 0.077 | 0.0742 | 0.0022 | 0.003 | 0.2155 | 0.0018 | 0.0133 | 0.0076 |
| SARS_CoV_2 | nsp2 | Cytoplasm/PM | Cytoplasm | Soluble | 0.0635 | 0.3293 | 0.0143 | 0.2246 | 0.0202 | 0.0157 | 0.1975 | 0.0136 | 0.1051 | 0.0162 |
| SARS_CoV_2 | nsp3 | | Endoplasmic | Membrane | 0.001 | 0.0004 | 0 | 0.0002 | 0.1113 | 0.7312 | 0.0002 | 0.0903 | 0.0651 | 0.0002 |
| | | | reticulum | | | | | | | | | | | |
| SARS_CoV_2 | nsp4 | ER | Cell | Membrane | 0 | 0 | 0.0001 | 0.0001 | 0.4961 | 0.0139 | 0 | 0.1846 | 0.3053 | 0 |
| | | | membrane | | | | | | | | | | | |
| SARS_CoV_2 | nsp5 | Cytoplasm/PM | Cytoplasm | Soluble | 0.0267 | 0.374 | 0.2223 | 0.2344 | 0.0109 | 0.0058 | 0.0735 | 0.0018 | 0.0081 | 0.0427 |
| SARS_CoV_2 | nsp6 | ER/Golgi | Golgi | Membrane | 0 | 0 | 0 | 0 | 0.1479 | 0.2928 | 0 | 0.3995 | 0.1597 | 0 |
| | | | apparatus | | | | | | | | | | | |
| SARS_CoV_2 | nsp7 | Cytoplasm/PM | Cytoplasm | Soluble | 0.2118 | 0.451 | 0.2854 | 0.0187 | 0.0055 | 0.0079 | 0.0002 | 0.0027 | 0.0168 | 0 |
| SARS_CoV_2 | nsp8 | Cytoplasm/PM | Cytoplasm | Soluble | 0.1572 | 0.5112 | 0.0112 | 0.0229 | 0.0243 | 0.029 | 0.0474 | 0.0167 | 0.0427 | 0.1374 |
| SARS_CoV_2 | nsp9 | Cytoplasm | Mitochondrion | Soluble | 0.0075 | 0.0541 | 0.0976 | 0.7034 | 0.0047 | 0.0046 | 0.1002 | 0.0007 | 0.0019 | 0.0253 |
| SARS_CoV_2 | nsp10 | Cytoplasm/PM | Extracellular | Soluble | 0.0362 | 0.1582 | 0.7092 | 0.058 | 0.0008 | 0.0009 | 0.0211 | 0.0005 | 0.0152 | 0 |
| SARS_CoV_2 | nsp11 | Cytoplasm/PM | Cytoplasm | Soluble | 0.0802 | 0.6554 | 0.028 | 0.0367 | 0.0309 | 0.0261 | 0.0189 | 0.028 | 0.0322 | 0.0636 |
| SARS_CoV_2 | nsp12 | PM/Cytoplasm | Cytoplasm | Soluble | 0.0802 | 0.6554 | 0.028 | 0.0367 | 0.0309 | 0.0261 | 0.0189 | 0.028 | 0.0322 | 0.0636 |
| SARS_CoV_2 | nsp13 | Cytoplasm/PM | Cytoplasm | Soluble | 0.2251 | 0.7146 | 0.0076 | 0.0132 | 0.0009 | 0.0011 | 0.0066 | 0.0027 | 0.007 | 0.0212 |
| SARS_CoV_2 | nsp14 | Cytoplasm/PM | Cytoplasm | Soluble | 0.0265 | 0.4667 | 0.3393 | 0.0543 | 0.0362 | 0.0132 | 0.018 | 0.0054 | 0.0375 | 0.0028 |
| SARS_CoV_2 | nsp15 | Cytoplasm/PM | Cytoplasm | Soluble | 0.0264 | 0.5939 | 0.1216 | 0.0665 | 0.0346 | 0.0105 | 0.0492 | 0.0089 | 0.084 | 0.0044 |
| SARS_CoV_2 | nsp16 | Cytoplasm/PM | Cytoplasm | Soluble | 0.0739 | 0.5956 | 0.1259 | 0.0822 | 0.013 | 0.0089 | 0.0301 | 0.0033 | 0.0247 | 0.0422 |
| SARS_CoV_2 | orf3a | Endosomes/ | Cell | Membrane | 0.0017 | 0.0018 | 0.0021 | 0.0081 | 0.3085 | 0.2825 | 0.0187 | 0.0873 | 0.2843 | 0.005 |
| | | PM/ER/Golgi | membrane | | | | | | | | | | | |
| SARS_CoV_2 | orf3b | Golgi | Extracellular | Soluble | 0.0441 | 0.0654 | 0.8442 | 0.0369 | 0.0006 | 0.003 | 0.0053 | 0.0002 | 0.0001 | 0 |
| SARS_CoV_2 | orf6 | Golgi/ | Mitochondrion | Membrane | 0.0944 | 0.0836 | 0.043 | 0.3963 | 0.0045 | 0.2919 | 0.0023 | 0.0415 | 0.0211 | 0.0214 |
| | | Punctate.cytoplasm/ | | | | | | | | | | | | |
| | | ER | | | | | | | | | | | | |
| SARS_CoV_2 | orf7a | Golgi/ | Endoplasmic | Membrane | 0 | 0 | 0.0435 | 0 | 0.2771 | 0.4259 | 0 | 0.15 | 0.1034 | 0 |
| | | Punctate.cytoplasm | reticulum | | | | | | | | | | | |
| SARS_CoV_2 | orf7b | Cytoplasm/ | Extracellular | Soluble | 0 | 0 | 0.6715 | 0 | 0.0807 | 0.223 | 0 | 0.0061 | 0.0186 | 0 |
| | | ER/PM | | | | | | | | | | | | |
| SARS_CoV_2 | orf8 | ER/Golgi | Extracellular | Soluble | 0 | 0 | 1 | 0 | 0 | 0 | 0 | 0 | 0 | 0 |
| SARS_CoV_2 | orf9b | Mitochondria/ | Cytoplasm | Soluble | 0.315 | 0.3329 | 0.0494 | 0.2466 | 0.0036 | 0.0023 | 0.038 | 0.0013 | 0.0097 | 0.0011 |
| | | Cytoplasm | | | | | | | | | | | | |
| SARS_CoV_2 | orf10 | ER | Extracellular | Soluble | 0.0036 | 0.0236 | 0.583 | 0.2761 | 0.0151 | 0.0515 | 0.0076 | 0.0137 | 0.0257 | 0.0002 |
| SARS_CoV_2 | M | Golgi/ER | Endoplasmic | Membrane | 0.0001 | 0 | 0 | 0.0063 | 0.0531 | 0.6787 | 0.0001 | 0.2525 | 0.0069 | 0.0024 |
| | | | reticulum | | | | | | | | | | | |
| SARS_CoV_2 | E | Golgi/ER | Golgi | Membrane | 0.0002 | 0.0001 | 0.0005 | 0.0047 | 0.1943 | 0.2792 | 0.0008 | 0.4642 | 0.0558 | 0.0002 |
| | | | apparatus | | | | | | | | | | | |
| SARS_CoV_2 | N | Cytoplasm/PM | Cytoplasm | Soluble | 0.1641 | 0.8223 | 0.0016 | 0.0013 | 0.0024 | 0.0006 | 0.0006 | 0.0004 | 0.0008 | 0.0059 |
| SARS_CoV_2 | S | PM/ER/Golgi | Cell | Membrane | 0 | 0 | 0.0358 | 0.0001 | 0.861 | 0.0764 | 0.0001 | 0.0152 | 0.0114 | 0 |
| | | | membrane | | | | | | | | | | | |
| SARS_CoV_2 | Protein | Cytoplasm? | Cell | Soluble | 0.0425 | 0.0819 | 0.2981 | 0.0324 | 0.4042 | 0.0349 | 0.0137 | 0.0125 | 0.0453 | 0.0345 |
| | 14 | | membrane | | | | | | | | | | | |
| MERS | nsp1 | Cytoplasm | Mitochondrion | Soluble | 0.0414 | 0.3415 | 0.0181 | 0.3929 | 0.0034 | 0.0027 | 0.1068 | 0.0006 | 0.0027 | 0.0898 |
| MERS | nsp2 | Cytoplasm/PM | Cytoplasm | Soluble | 0.0227 | 0.7471 | 0.0157 | 0.0039 | 0.0112 | 0.013 | 0.0037 | 0.0005 | 0.0374 | 0.1448 |
| MERS | nsp3 | ER | Endoplasmic | Membrane | 0.0003 | 0 | 0 | 0.0001 | 0.1541 | 0.7351 | 0.0001 | 0.0532 | 0.0568 | 0.0003 |
| | | | reticulum | | | | | | | | | | | |
| MERS | nsp3_C740A | ER | Endoplasmic | Membrane | 0.0003 | 0 | 0 | 0.0001 | 0.1582 | 0.7347 | 0.0001 | 0.05 | 0.0563 | 0.0002 |
| | | | reticulum | | | | | | | | | | | |
| MERS | nsp4 | ER | Lysosome/ | Membrane | 0 | 0 | 0.0001 | 0.0002 | 0.308 | 0.0564 | 0 | 0.2675 | 0.3678 | 0 |
| | | | Vacuole | | | | | | | | | | | |
| MERS | nsp5 | PM/Cytoplasm | Cytoplasm | Soluble | 0.0238 | 0.3952 | 0.2154 | 0.2102 | 0.0119 | 0.0077 | 0.0707 | 0.0019 | 0.0109 | 0.0524 |
| MERS | nsp5_C148A | Cytoplasm/PM | Cytoplasm | Soluble | 0.0242 | 0.4124 | 0.2004 | 0.2122 | 0.0103 | 0.0066 | 0.0685 | 0.0017 | 0.0092 | 0.0546 |
| MERS | nsp6 | ER/Golgi | Golgi | Membrane | 0 | 0 | 0 | 0.0001 | 0.2288 | 0.238 | 0 | 0.3353 | 0.1979 | 0 |
| | | | apparatus | | | | | | | | | | | |
| MERS | nsp7 | Cytoplasm/PM | Cytoplasm | Soluble | 0.2028 | 0.4393 | 0.3043 | 0.0127 | 0.0052 | 0.0111 | 0.0001 | 0.0033 | 0.021 | 0 |
| MERS | nsp8 | Cytoplasm/PM | Cytoplasm | Soluble | 0.095 | 0.5973 | 0.0169 | 0.0141 | 0.0232 | 0.0124 | 0.0169 | 0.0222 | 0.1355 | 0.0665 |
| MERS | nsp9 | Cytoplasm/PM | Cytoplasm | Soluble | 0.1298 | 0.4833 | 0.0817 | 0.2594 | 0.004 | 0.0011 | 0.006 | 0.0003 | 0.0022 | 0.0322 |
| MERS | nsp10 | Cytoplasm/PM | Cytoplasm | Soluble | 0.1321 | 0.4525 | 0.3243 | 0.0648 | 0.002 | 0.0003 | 0.0195 | 0.0002 | 0.0041 | 0.0003 |
| MERS | nsp11 | Cytoplasm/PM | Extracellular | Soluble | 0.1388 | 0.0938 | 0.4007 | 0.0684 | 0.0097 | 0.0551 | 0.0134 | 0.0396 | 0.1803 | 0.0002 |
| MERS | nsp12 | ER/Cytoplasm | Cytoplasm | Soluble | 0.0695 | 0.7999 | 0.0101 | 0.0156 | 0.0119 | 0.0153 | 0.0042 | 0.0109 | 0.0223 | 0.0403 |
| MERS | nsp13 | Mitochondria/ | Cytoplasm | Soluble | 0.2662 | 0.6154 | 0.0035 | 0.0376 | 0.0009 | 0.0017 | 0.0467 | 0.0071 | 0.0088 | 0.012 |
| | | PM | | | | | | | | | | | | |
| MERS | nsp14 | Cytoplasm/PM | Cytoplasm | Soluble | 0.0389 | 0.4338 | 0.372 | 0.0393 | 0.038 | 0.0091 | 0.0085 | 0.0038 | 0.0483 | 0.0083 |
| MERS | nsp15 | Cytoplasm/PM | Cytoplasm | Soluble | 0.0111 | 0.5548 | 0.1849 | 0.0686 | 0.0426 | 0.0106 | 0.0411 | 0.0051 | 0.0697 | 0.0115 |
| MERS | nsp16 | Cytoplasm/PM | Cytoplasm | Soluble | 0.0668 | 0.5771 | 0.1171 | 0.1087 | 0.0173 | 0.0101 | 0.019 | 0.002 | 0.011 | 0.0709 |
| MERS | orf3 | Golgi/ER | Extracellular | Soluble | 0.0009 | 0.0063 | 0.8522 | 0.0037 | 0.0046 | 0.0766 | 0.0005 | 0.0139 | 0.0414 | 0.0001 |
| MERS | orf4a | Cytoplasm/PM | Extracellular | Soluble | 0.1353 | 0.1664 | 0.4515 | 0.1801 | 0.0194 | 0.0083 | 0.0104 | 0.01 | 0.0166 | 0.002 |
| MERS | orf4b | Cytoplasm | Nucleus | Soluble | 0.7193 | 0.2717 | 0.0022 | 0.0022 | 0.0016 | 0.0003 | 0.0002 | 0.0003 | 0.0004 | 0.0018 |
| MERS | orf5 | Golgi/ER/ | Cell | Membrane | 0.0013 | 0.0002 | 0.0003 | 0.0069 | 0.435 | 0.168 | 0.0738 | 0.0754 | 0.2365 | 0.0027 |
| | | Punctate.cytoplasm | membrane | | | | | | | | | | | |
| MERS | orf8b | ER/ | Mitochondrion | Soluble | 0.151 | 0.1586 | 0.0011 | 0.4053 | 0.0031 | 0.02 | 0.0341 | 0.0142 | 0.0008 | 0.2117 |
| | | Punctate.cytoplasm | | | | | | | | | | | | |
| MERS | M | Golgi/ER | Endoplasmic | Membrane | 0.0004 | 0 | 0 | 0.002 | 0.1512 | 0.3733 | 0.0002 | 0.1958 | 0.2769 | 0.0001 |
| | | | reticulum | | | | | | | | | | | |
| MERS | E | Golgi/ER | Golgi | Membrane | 0.0025 | 0.0013 | 0.0268 | 0.0803 | 0.2152 | 0.1817 | 0.0029 | 0.404 | 0.0844 | 0.0007 |
| | | | apparatus | | | | | | | | | | | |
| MERS | N | Cytoplasm/PM | Cytoplasm | Soluble | 0.2302 | 0.7106 | 0.0043 | 0.0095 | 0.0092 | 0.0018 | 0.0089 | 0.0041 | 0.0052 | 0.0164 |
| MERS | S | PM/ER/Golgi | Cell | Membrane | 0 | 0 | 0.0091 | 0.0001 | 0.9012 | 0.059 | 0 | 0.0251 | 0.0055 | 0 |
| | | | membrane | | | | | | | | | | | |
| SARS_CoV_1 | nsp1 | Cytoplasm/PM | Cytoplasm | Soluble | 0.1375 | 0.4535 | 0.0756 | 0.0878 | 0.0022 | 0.0033 | 0.221 | 0.0013 | 0.0106 | 0.0073 |
| SARS_CoV_1 | nsp2 | Cytoplasm/PM | Cytoplasm | Soluble | 0.1926 | 0.6754 | 0.0058 | 0.0051 | 0.0238 | 0.0042 | 0.0069 | 0.0022 | 0.0182 | 0.066 |
| SARS_CoV_1 | nsp3 | | Endoplasmic | Membrane | 0.0012 | 0 | 0 | 0.0002 | 0.1023 | 0.7627 | 0.0001 | 0.0787 | 0.0542 | 0.0005 |
| | | | reticulum | | | | | | | | | | | |
| SARS_CoV_1 | nsp4 | ER | Cell | Membrane | 0 | 0 | 0.0002 | 0.0001 | 0.4294 | 0.0398 | 0 | 0.1692 | 0.3613 | 0 |
| | | | membrane | | | | | | | | | | | |
| SARS_CoV_1 | nsp5 | Cytoplasm/PM | Cytoplasm | Soluble | 0.0247 | 0.3879 | 0.2182 | 0.2269 | 0.0102 | 0.0055 | 0.0732 | 0.0016 | 0.0077 | 0.0441 |
| SARS_CoV_1 | nsp6 | ER/Golgi | Golgi | Membrane | 0 | 0 | 0 | 0 | 0.16 | 0.2951 | 0 | 0.3887 | 0.1561 | 0 |
| | | | apparatus | | | | | | | | | | | |
| SARS_CoV_1 | nsp7 | Cytoplasm/PM | Cytoplasm | Soluble | 0.2054 | 0.4641 | 0.2816 | 0.0171 | 0.0055 | 0.0073 | 0.0001 | 0.0026 | 0.0163 | 0 |
| SARS_CoV_1 | nsp8 | Cytoplasm/PM | Cytoplasm | Soluble | 0.1116 | 0.5879 | 0.0102 | 0.0174 | 0.0153 | 0.0123 | 0.0523 | 0.0061 | 0.0336 | 0.1532 |
| SARS_CoV_1 | nsp9 | Cytoplasm/PM | Mitochondrion | Soluble | 0.0096 | 0.0648 | 0.087 | 0.7042 | 0.0038 | 0.0038 | 0.0996 | 0.0006 | 0.0017 | 0.025 |
| SARS_CoV_1 | nsp10 | PM/Cytoplasm | Extracellular | Soluble | 0.0386 | 0.1676 | 0.6966 | 0.0548 | 0.0007 | 0.001 | 0.0217 | 0.0005 | 0.0185 | 0 |
| SARS_CoV_1 | nsp11 | Cytoplasm/PM | Extracellular | Soluble | 0.031 | 0.1003 | 0.3883 | 0.1191 | 0.0032 | 0.0021 | 0.2754 | 0.0035 | 0.0762 | 0.001 |
| SARS_CoV_1 | nsp12 | Cytoplasm/PM | Cytoplasm | Soluble | 0.0755 | 0.6164 | 0.0296 | 0.0353 | 0.033 | 0.027 | 0.0202 | 0.0288 | 0.0354 | 0.0988 |
| SARS_CoV_1 | nsp13 | Cytoplasm/PM | Cytoplasm | Soluble | 0.2188 | 0.6512 | 0.0119 | 0.0456 | 0.0016 | 0.0015 | 0.0281 | 0.0059 | 0.0105 | 0.0249 |
| SARS_CoV_1 | nsp14 | Cytoplasm/PM | Cytoplasm | Soluble | 0.0239 | 0.4537 | 0.353 | 0.0534 | 0.0371 | 0.0131 | 0.018 | 0.0058 | 0.0391 | 0.0027 |
| SARS_CoV_1 | nsp15 | Cytoplasm/PM | Cytoplasm | Soluble | 0.0309 | 0.5892 | 0.1558 | 0.0571 | 0.029 | 0.0102 | 0.04 | 0.0069 | 0.0759 | 0.005 |
| SARS_CoV_1 | nsp16 | Cytoplasm/PM | Cytoplasm | Soluble | 0.0835 | 0.6592 | 0.0452 | 0.1241 | 0.0039 | 0.0032 | 0.0269 | 0.0015 | 0.0075 | 0.0449 |
| SARS_CoV_1 | orf3a | Endosomes/ | Lysosome/ | Membrane | 0.0038 | 0.0061 | 0.0056 | 0.0197 | 0.1833 | 0.2503 | 0.0704 | 0.064 | 0.3838 | 0.013 |
| | | PM/ER/Golgi | Vacuole | | | | | | | | | | | |
| SARS_CoV_1 | orf3b | Cytoplasm | Mitochondrion | Soluble | 0.1842 | 0.0969 | 0.2131 | 0.417 | 0.0023 | 0.0012 | 0.0803 | 0.0008 | 0.0021 | 0.0021 |
| SARS_CoV_1 | orf6 | ER/Golgi/ | Extracellular | Soluble | 0.0474 | 0.0566 | 0.4547 | 0.2286 | 0.0289 | 0.0859 | 0.0443 | 0.0097 | 0.043 | 0.0008 |
| | | Punctate.cytoplasm | | | | | | | | | | | | |
| SARS_CoV_1 | orf7a | Golgi/ | Endoplasmic | Membrane | 0 | 0 | 0.046 | 0 | 0.2457 | 0.5195 | 0 | 0.1501 | 0.0386 | 0 |
| | | Punctate.cytoplasm | reticulum | | | | | | | | | | | |
| SARS_CoV_1 | orf7b | Cytoplasm/ | Endoplasmic | Soluble | 0 | 0 | 0.3566 | 0 | 0.1089 | 0.4074 | 0 | 0.0888 | 0.0382 | 0 |
| | | ER/Golgi/PM | reticulum | | | | | | | | | | | |
| SARS_CoV_1 | orf8a | ER | Extracellular | Soluble | 0 | 0 | 1 | 0 | 0 | 0 | 0 | 0 | 0 | 0 |
| SARS_CoV_1 | orf8b | Cytoplasm/PM | Mitochondrion | Soluble | 0.0298 | 0.3311 | 0.2398 | 0.3947 | 0.0018 | 0.0009 | 0.0012 | 0.0002 | 0.0003 | 0.0001 |
| SARS_CoV_1 | orf9b | Mitochondria/ | Cytoplasm | Soluble | 0.3145 | 0.3327 | 0.052 | 0.2153 | 0.008 | 0.0046 | 0.0516 | 0.0028 | 0.0172 | 0.0013 |
| | | Cytoplasm | | | | | | | | | | | | |
| SARS_CoV_1 | orf9c | Cytoplasm | Extracellular | Soluble | 0.1527 | 0.2688 | 0.3169 | 0.2104 | 0.0103 | 0.0098 | 0.0143 | 0.0068 | 0.0067 | 0.0033 |
| SARS_CoV_1 | M | Golgi/ER | Endoplasmic | Membrane | 0.0005 | 0 | 0.0001 | 0.0018 | 0.2185 | 0.3524 | 0.0005 | 0.1442 | 0.2817 | 0.0002 |
| | | | reticulum | | | | | | | | | | | |
| SARS_CoV_1 | E | Golgi/ER | Golgi | Membrane | 0.0005 | 0.0003 | 0.0018 | 0.0045 | 0.2636 | 0.1873 | 0.0022 | 0.4122 | 0.1272 | 0.0004 |
| | | | apparatus | | | | | | | | | | | |
| SARS_CoV_1 | N | Cytoplasm/PM | Cytoplasm | Soluble | 0.2015 | 0.7728 | 0.006 | 0.0012 | 0.0078 | 0.0014 | 0.0008 | 0.0015 | 0.0021 | 0.005 |
| SARS_CoV_1 | S | PM/ER/Golgi | Cell | Membrane | 0 | 0 | 0.0532 | 0.0001 | 0.8413 | 0.0789 | 0.0002 | 0.0139 | 0.0123 | 0 |
| | | | membrane | | | | | | | | | | | |

TABLE 8D
UNIPROT ANNOTATION

| | | UNIPROT LOCATION INFO | | | | | | | |
|---|---|---|---|---|---|---|---|---|---|
| | Experimental | | signal | other loc | | LocSigDB (http://genome.unmc.edu/LocSigDB/index.html) | | | |
| protein | Location | uniprot link | peptide | signals | uniprot location | Signal | Coordinates | Localization | Virus |
| NSP1 | Cytoplasm/ | https://covid- | \— | \— | \— | Yx{2}[VILFWCM] | 67-71, 117-121, 153- | Lysosome | SARS_CoV_2 |
| | PM | 19.uniprot.org/ | | | | | 157 | | |
| | | uniprotkb/P0DTC1 | | | | Kx{3}Q | 10-15 | Lysosome | SARS_CoV_2 |
| | | | | | | [HK]x{1}K | 44-47 | Endoplasmic | SARS_CoV_2 |
| | | | | | | | | reticulum | |
| | | | | | | Lx{2}KN | 121-126 | Golgi (early | SARS_CoV_2 |
| | | | | | | | | post -golgi | |
| | | | | | | | | comparments) | |
| NSP2 | Cytoplasm/ | https://covid- | \— | \— | \— | [DE]x{3}L[LI] | 545-551 | Lysosome\|melanosome | SARS_CoV_2 |
| | PM | 19.uniprot.org/ | | | | Ex{3}LL | 545-551 | Lysosome | SARS_CoV_2 |
| | | uniprotkb/P0DTC1 | | | | Yx{2}[VILFWCM] | 233-237, 316-320, | Lysosome | SARS_CoV_2 |
| | | | | | | | 441-445, 537-541, | | |
| | | | | | | | 619-623 | | |
| | | | | | | Kx{3}Q | 317-322, 492-497 | Lysosome | SARS_CoV_2 |
| | | | | | | Dx{1}E | 615-618 | Endoplasmic | SARS_CoV_2 |
| | | | | | | | | reticulum | |
| | | | | | | [HK]x{1}K | 110-113, 237-240, | Endoplasmic | SARS_CoV_2 |
| | | | | | | | 276-279, 333-336, | reticulum | |
| | | | | | | | 443-446, 454-457, | | |
| | | | | | | | 519-522, 532-535 | | |
| NSP3 | | https://covid- | \— | \— | Host membrane: Multi- | [DE]x{3}L[LI] | 308-314 | Lysosome\|melanosome | SARS_CoV_2 |
| | | 19.uniprot.org/ | | | pass membrane protein, | Ex{3}LL | 308-314 | Lysosome | SARS_CoV_2 |
| | | uniprotkb/P0DTC1 | | | Host cytoplasm | Yx{2}[VILFWCM] | 18-22, 87-91, 103- | Lysosome | SARS_CoV_2 |
| | | | | | | | 107, 213-217, 317- | | |
| | | | | | | | 321, 356-360, 365- | | |
| | | | | | | | 369, 438-442, 588- | | |
| | | | | | | | 592, 693-697, 840- | | |
| | | | | | | | 844, 958-962, 1018- | | |
| | | | | | | | 1022, 1483-1487, | | |
| | | | | | | | 1513-1517, 1535- | | |
| | | | | | | | 1539, 1566-1570, | | |
| | | | | | | | 1573-1577, 1579- | | |
| | | | | | | | 1583, 1743-1747, | | |
| | | | | | | | 1859-1863 | | |
| | | | | | | Kx{3}Q | 376-381, 935-940, | Lysosome | SARS_CoV_2 |
| | | | | | | | 962-967, 977-982, | | |
| | | | | | | | 1838-1843 | | |
| | | | | | | GYx{2}[VILFWCM] | 17-22, 212-217 | Lysosome | SARS_CoV_2 |
| | | | | | | EED | 158-161 | Nucleus | SARS_CoV_2 |
| | | | | | | Dx{1}E | 112-115, 117-120, | Endoplasmic | SARS_CoV_2 |
| | | | | | | | 729-732, 1827-1830, | reticulum | |
| | | | | | | | 1844-1847 | | |
| | | | | | | [HK]x{1}K | 233-236, 413-416, | Endoplasmic | SARS_CoV_2 |
| | | | | | | | 530-533, 587-590, | reticulum | |
| | | | | | | | 788-791, 834-837, | | |
| | | | | | | | 837-840, 1017-1020, | | |
| | | | | | | | 1728-1731, 1790- | | |
| | | | | | | | 1793 | | |
| | | | | | | Yx{4}LL | 857-864, 1353-1360 | Golgi | SARS_CoV_2 |
| NSP4 | ER | https://covid- | \— | \— | Host membrane: Multi- | [DE]x{3}L[LI] | 275-281 | Lysosome\|melanosome | SARS_CoV_2 |
| | | 19.uniprot.org/ | | | pass membrane protein, | Yx{2}[VILFWCM] | 62-66, 158-162, 198- | Lysosome | SARS_CoV_2 |
| | | uniprotkb/P0DTC1 | | | Host cytoplasm | | 202, 207-211, 264- | | |
| | | | | | Localizes in virally- | | 268, 315-319, 327- | | |
| | | | | | induced cytoplasmic | | 331, 351-355, 358- | | |
| | | | | | double-membrane vesicles | | 362, 362-366, 397- | | |
| | | | | | | | 401, 407-411, 443- | | |
| | | | | | | | 447, 460-464, 467- | | |
| | | | | | | | 471 | | |
| | | | | | | GYx{2}I | 61-66 | Lysosome | SARS_CoV_2 |
| | | | | | | GYx{2}[VILFWCM] | 61-66 | Lysosome | SARS_CoV_2 |
| | | | | | | Dx{1}E | 233-236 | Endoplasmic | SARS_CoV_2 |
| | | | | | | | | reticulum | |
| | | | | | | [HK]x{1}K | 466-469 | Endoplasmic | SARS_CoV_2 |
| | | | | | | | | reticulum | |
| NSP5 | Cytoplasm/ | \— | \— | \— | \— | Yx{2}[VILFWCM] | 54-58, 101-105, 154- | Lysosome | SARS_CoV_2 |
| | PM | | | | | | 158, 182-186, 209- | | |
| | | | | | | | 213, 239-243 | | |
| | | | | | | Kx{3}Q | 269-274 | Lysosome | SARS_CoV_2 |
| | | | | | | SPS | 121-124 | Nucleus | SARS_CoV_2 |
| | | | | | | Dx{1}E | 176-179 | Endoplasmic | SARS_CoV_2 |
| | | | | | | | | reticulum | |
| | | | | | | [HK]x{1}K | 88-91, 100-103 | Endoplasmic | SARS_CoV_2 |
| | | | | | | | | reticulum | |
| NSP6 | ER/Golgi | https://covid- | \— | \— | Host membrane: Multi- | Yx{2}[VILFWCM] | 80-84, 175-179, 196- | Lysosome | SARS_CoV_2 |
| | | 19.uniprot.org/ | | | pass membrane protein | | 200, 214-218, 224- | | |
| | | uniprotkb/P0DTC1 | | | | | 228, 234-238, 242- | | |
| | | | | | | | 246 | | |
| | | | | | | [HK]x{1}K | 61-64, 109-112 | Endoplasmic | SARS_CoV_2 |
| | | | | | | | | reticulum | |
| | | | | | | Lx{2}KN | 260-265 | Golgi (early | SARS_CoV_2 |
| | | | | | | | | post -golgi | |
| | | | | | | | | comparments) | |
| NSP7 | Cytoplasm/ | https://covid- | \— | \— | Host cytoplasm, host | Kx{3}Q | 27-32 | Lysosome | SARS_CoV_2 |
| | PM | 19.uniprot.org/ | | | perinuclear region | | | | |
| | | uniprotkb/P0DTC1 | | | nsp7, nsp8, nsp9 and | | | | |
| | | | | | nsp10 are localized in | | | | |
| | | | | | cytoplasmic foci, largely | | | | |
| | | | | | perinuclear. Late in | | | | |
| | | | | | infection, they merge into | | | | |
| | | | | | confluent complexes | | | | |
| NSP8 | Cytoplasm/ | https://covid- | \— | S | Host cytoplasm, host | Yx{2}[VILFWCM] | 12-16 | Lysosome | SARS_CoV_2 |
| | PM | 19.uniprot.org/ | | | perinuclear region | Kx{3}Q | 61-66 | Lysosome | SARS_CoV_2 |
| | | uniprotkb/P0DTC1 | | | nsp7, nsp8, nsp9 and | KKLKK | 36-41 | Nucleus | SARS_CoV_2 |
| | | | | | nsp10 are localized in | Dx{1}E | 30-33 | Endoplasmic | SARS_CoV_2 |
| | | | | | cytoplasmic foci, largely | | | reticulum | |
| | | | | | perinuclear. Late in | [HK]x{1}K | 37-40 | Endoplasmic | SARS_CoV_2 |
| | | | | | infection, they merge into | | | reticulum | |
| | | | | | confluent complexes | | | | |
| NSP9 | Cytoplasm | https://covid- | \— | \— | Host cytoplasm, host | Yx{2}[VILFWCM] | 66-70, 87-91 | Lysosome | SARS_CoV_2 |
| | | 19.uniprot.org/ | | | perinuclear region | [HK]x{1}K | 84-87 | Endoplasmic | SARS_CoV_2 |
| | | uniprotkb/P0DTC1 | | | nsp7, nsp8, nsp9 and | | | reticulum | |
| | | | | | nsp10 are localized in | | | | |
| | | | | | cytoplasmic foci, largely | | | | |
| | | | | | perinuclear. Late in | | | | |
| | | | | | infection, they merge into | | | | |
| | | | | | confluent complexes | | | | |
| NSP10 | Cytoplasm/ | https://covid- | \— | \— | Host cytoplasm, host | Yx{2}[VILFWCM] | 76-80, 96-100 | Lysosome | SARS_CoV_2 |
| | PM | 19.uniprot.org/ | | | perinuclear region | Dx{1}E | 64-67 | Endoplasmic | SARS_CoV_2 |
| | | uniprotkb/P0DTC1 | | | nsp7, nsp8, nsp9 and | | | reticulum | |
| | | | | | nsp10 are localized in | [HK]x{1}K | 93-96 | Endoplasmic | SARS_CoV_2 |
| | | | | | cytoplasmic foci, largely | | | reticulum | |
| | | | | | perinuclear. Late in | | | | |
| | | | | | infection, they merge into | | | | |
| | | | | | confluent complexes | | | | |
| NSP11 | Cytoplasm/ | \— | \— | \— | \— | \— | \— | \— | SARS_CoV_2 |
| | PM | | | | | | | | |
| NSP12 | PM/ | \— | \— | \— | \— | [DE]x{3}L[LI] | 61-67, 465-471 | Lysosome\|melanosome | SARS_CoV_2 |
| | Cytoplasm | | | | | Yx{2}[VILFWCM] | 32-36, 69-73, 87-91, | Lysosome | SARS_CoV_2 |
| | | | | | | | 149-153, 163-167, | | |
| | | | | | | | 175-179, 237-241, | | |
| | | | | | | | 265-269, 479-483, | | |
| | | | | | | | 516-520, 595-599, | | |
| | | | | | | | 606-610, 619-623, | | |
| | | | | | | | 728-732, 746-750, | | |
| | | | | | | | 826-830, 877-881, | | |
| | | | | | | | 903-907, 921-925 | | |
| | | | | | | Kx{3}Q | 288-293, 871-876 | Lysosome | SARS_CoV_2 |
| | | | | | | Dx{1}E | 608-611 | Endoplasmic | SARS_CoV_2 |
| | | | | | | | | reticulum | |
| | | | | | | [HK]x{1}K | 572-575 | Endoplasmic | SARS_CoV_2 |
| | | | | | | | | reticulum | |
| | | | | | | Yx{4}LL | 265-272 | Golgi | SARS_CoV_2 |
| | | | | | | SVM | 904-907 | Plasma | SARS_CoV_2 |
| | | | | | | | | membrane | |
| | | | | | | YEDQ | 521-525 | Plasma | SARS_CoV_2 |
| | | | | | | | | membrane | |
| NSP13 | Cytoplasm/ | \— | \— | \— | \— | Yx{2}[VILFWCM] | 31-35, 224-228, 246- | Lysosome | SARS_CoV_2 |
| | PM | | | | | | 250, 253-257, 269- | | |
| | | | | | | | 273, 277-281, 306- | | |
| | | | | | | | 310, 324-328, 355- | | |
| | | | | | | | 359, 396-400, 476- | | |
| | | | | | | | 480, 541-545, 582- | | |
| | | | | | | | 586 | | |
| | | | | | | Kx{3}Q | 271-276 | Lysosome | SARS_CoV_2 |
| | | | | | | PPx{2}R | 174-179 | Nucleus | SARS_CoV_2 |
| | | | | | | Dx{1}E | 160-163 | Endoplasmic | SARS_CoV_2 |
| | | | | | | | | reticulum | |
| | | | | | | [HK]x{1}K | 345-348, 460-463, | Endoplasmic | SARS_CoV_2 |
| | | | | | | | 465-468 | reticulum | |
| NSP14 | Cytoplasm/ | \— | \— | \— | \— | Yx{2}[VILFWCM] | 50-54, 68-72, 153- | Lysosome | SARS_CoV_2 |
| | PM | | | | | | 157, 223-227, 236- | | |
| | | | | | | | 240, 259-263, 295- | | |
| | | | | | | | 299, 464-468, 497- | | |
| | | | | | | | 501, 510-514, 516- | | |
| | | | | | | | 520 | | |
| | | | | | | Kx{3}Q | 60-65, 338-343 | Lysosome | SARS_CoV_2 |
| | | | | | | GYx{2}[VILFWCM] | 67-72 | Lysosome | SARS_CoV_2 |
| | | | | | | Dx{1}E | 89-92, 344-347 | Endoplasmic | SARS_CoV_2 |
| | | | | | | | | reticulum | |
| | | | | | | [HK]x{1}K | 31-34, 454-457 | Endoplasmic | SARS_CoV_2 |
| | | | | | | | | reticulum | |
| | | | | | | YKGL | 153-157 | Golgi | SARS_CoV_2 |
| NSP15 | Cytoplasm/ | \— | \— | \— | \— | Yx{2}[VILFWCM] | 32-36, 179-183, 237- | Lysosome | SARS_CoV_2 |
| | PM | | | | | | 241, 324-328, 342- | | |
| | | | | | | | 346 | | |
| | | | | | | Kx{3}Q | 204-209 | Lysosome | SARS_CoV_2 |
| | | | | | | Dx{1}E | 39-42 | Endoplasmic | SARS_CoV_2 |
| | | | | | | | | reticulum | |
| NSP16 | Cytoplasm/ | \— | \— | \— | \— | Yx{2}[VILFWCM] | 47-51, 181-185, 228- | Lysosome | SARS_CoV_2 |
| | PM | | | | | | 232, 242-246 | | |
| | | | | | | Kx{3}Q | 24-29, 214-219 | Lysosome | SARS_CoV_2 |
| | | | | | | [HK]x{1}K | 135-138 | Endoplasmic | SARS_CoV_2 |
| | | | | | | | | reticulum | |
| E | Golgi/ER | https://covid- | \— | The | Host Golgi apparatus | [DE]x{3}L[LI] | 7-13 | Lysosome\|melanosome | SARS_CoV_2 |
| | | 19.uniprot.org/ | | cytoplasmic | membrane: Single-pass | Yx{2}[VILFWCM] | 1-5, 58-62 | Lysosome | SARS_CoV_2 |
| | | uniprotkb/P0DTC4 | | tail | type III membrane protein | | | | |
| | | | | functions | | | | | |
| | | | | as a Golgi | | | | | |
| | | | | complex- | | | | | |
| | | | | targeting | | | | | |
| | | | | signal | | | | | |
| M | Golgi/ER | https://covid- | \— | \— | Virion membrane: Multi- | [DE]x{3}L[LI] | 11-17, 114-120, 214- | Lysosome\|melanosome | SARS_CoV_2 |
| | | 19.uniprot.org/ | | | pass membrane protein | | 220 | | |
| | | uniprotkb/P0DTC5 | | | Host Golgi apparatus | Ex{3}LL | 11-17, 114-120 | Lysosome | SARS_CoV_2 |
| | | | | | membrane: Multi-pass | | | | |
| | | | | | membrane protein | | | | |
| | | | | | Largely embedded in the | Yx{2}[VILFWCM] | 177-181 | Lysosome | SARS_CoV_2 |
| | | | | | lipid bilayer | | | | |
| | | | | | | Kx{3}Q | 14-19 | Lysosome | SARS_CoV_2 |
| N | Cytoplasm/ | https://covid- | \— | \— | Virion | [DE]x{3}L[LI] | 347-353 | Lysosome\|melanosome | SARS_CoV_2 |
| | PM | 19.uniprot.org/ | | | Host endoplasmic | Yx{2}[VILFWCM] | 297-301, 359-363 | Lysosome | SARS_CoV_2 |
| | | uniprotkb/P0DTC9 | | | reticulum-Golgi | | | | |
| | | | | | intermediate compartment | | | | |
| | | | | | Host Golgi apparatus | Kx{3}Q | 236-241, 255-260, | Lysosome | SARS_CoV_2 |
| | | | | | | | 298-303, 404-409 | | |
| | | | | | Located inside the virion, | Dx{1}E | 287-290 | Endoplasmic | SARS_CoV_2 |
| | | | | | complexed with the viral | | | reticulum | |
| | | | | | RNA. Probably associates | | | | |
| | | | | | with ER-derived | | | | |
| | | | | | membranes where it | | | | |
| | | | | | participates in viral RNA | | | | |
| | | | | | synthesis and virus | | | | |
| | | | | | budding | | | | |
| | | | | | | SKK | 254-257 | Endoplasmic | SARS_CoV_2 |
| | | | | | | | | reticulum | |
| | | | | | | [HK]x{1}K | 58-61, 99-102, 369- | Endoplasmic | SARS_CoV_2 |
| | | | | | | | 372, 372-375 | reticulum | |
| ORF3a | Endosomes/ | https://covid- | \— | \— | Virion | Yx{2}[VILFWCM] | 90-94, 108-112, 144- | Lysosome | SARS_CoV_2 |
| | PM/ER/ | 19.uniprot.org/ | | | | | 148, 153-157, 159- | | |
| | Golgi | uniprotkb/P0DTC3 | | | | | 163, 210-214, 232- | | |
| | | | | | | | 236 | | |
| | | | | | Host Golgi apparatus | Kx{3}Q | 65-70 | Lysosome | SARS_CoV_2 |
| | | | | | membrane: Multi-pass | | | | |
| | | | | | membrane protein | | | | |
| | | | | | Host cell membrane: | | | | SARS_CoV_2 |
| | | | | | Multi-pass membrane | | | | |
| | | | | | protein | | | | |
| | | | | | Secreted | | | | SARS_CoV_2 |
| | | | | | Host cytoplasm | | | | SARS_CoV_2 |
| | | | | | The cell surface expressed | | | | SARS_CoV_2 |
| | | | | | protein can undergo | | | | |
| | | | | | endocytosis. The protein is | | | | |
| | | | | | secreted in association | | | | |
| | | | | | with membranous | | | | |
| | | | | | structures | | | | |
| ORF6 | Golgi/ | https://covid- | \— | \— | Host endoplasmic | Yx{2}[VILFWCM] | 48-52 | Lysosome | SARS_CoV_2 |
| | UM/ER | 19.uniprot.org/ | | | reticulum membrane | | | | |
| | | uniprotkb/P0DTC6 | | | Host Golgi apparatus | Dx{1}E | 52-55 | Endoplasmic | SARS_CoV_2 |
| | | | | | membrane | | | reticulum | |
| | | | | | Host cytoplasm | Lx{2}KN | 34-39 | Golgi (early | SARS_CoV_2 |
| | | | | | | | | post -golgi | |
| | | | | | | | | comparments) | |
| | | | | | Localizes to virus-induced | | | | SARS_CoV_2 |
| | | | | | vesicular structures called | | | | |
| | | | | | double membrane vesicles | | | | |
| ORF7a | Golgi/UM | https://covid- | positions | \— | Virion | Yx{2}[VILFWCM] | 19-23, 96-100 | Lysosome | SARS_CoV_2 |
| | | 19.uniprot.org/ | 1-15 | | Host endoplasmic | Kx{3}Q | 71-76 | Lysosome | SARS_CoV_2 |
| | | uniprotkb/P0DTC7 | | | reticulum membrane: | | | | |
| | | | | | Single-pass membrane | | | | |
| | | | | | protein | | | | |
| | | | | | Host endoplasmic | KRK | 116-119 | Nucleus | SARS_CoV_2 |
| | | | | | reticulum-Golgi | | | | |
| | | | | | intermediate compartment | | | | |
| | | | | | membrane: Single-pass | | | | |
| | | | | | type I membrane protein | | | | |
| | | | | | Host Golgi apparatus | [HK]x{1}K | 116-119 | Endoplasmic | SARS_CoV_2 |
| | | | | | membrane: Single-pass | | | reticulum | |
| | | | | | membrane protein | | | | |
| ORF8 | ER/Golgi | https://covid- | positions | \— | \— | Yx{2}[VILFWCM] | 41-45, 45-49, 72-76, | Lysosome | SARS_CoV_2 |
| | | 19.uniprot.org/ | 1-15 | | | | 104-108, 110-114 | | |
| | | uniprotkb/P0DTC8 | | | | Kx{3}Q | 67-72 | Lysosome | SARS_CoV_2 |
| ORF9b | Mitochondria/ | https://covid- | \— | 45-54: | Virion | Yx{2}[VILFWCM] | 41-45 | Lysosome | SARS_CoV_2 |
| | Cytoplasm | 19.uniprot.org/ | | nuclear | Host cytoplasmic vesicle | | | | SARS_CoV_2 |
| | | uniprotkb/P0DTC2 | | export | membrane: Peripheral | | | | |
| | | | | signal | membrane protein | | | | |
| | | | | | Host cytoplasm | | | | SARS_CoV_2 |
| | | | | | Host endoplasmic | | | | SARS_CoV_2 |
| | | | | | reticulum | | | | |
| | | | | | Host nucleus | | | | SARS_CoV_2 |
| | | | | | Host mitochondrion | | | | SARS_CoV_2 |
| | | | | | Binds non-covalently to | | | | SARS_CoV_2 |
| | | | | | intracellular lipid bilayers | | | | |
| ORF10 | ER | https://covid- | \— | \— | \— | Yx{2}[VILFWCM] | 2-6, 13-17 | Lysosome | SARS_CoV_2 |
| | | 19.uniprot.org/ | | | | GYx{2}[VILFWCM] | 1-6 | Lysosome | SARS_CoV_2 |
| | | uniprotkb/A0A663DJA2 | | | | | | | |
| S | PM/ER/ | https://covid- | positions | \— | Virion membrane | [DE]x{3}L[LI] | 747-753, 917-923 | Lysosome\|melanosome | SARS_CoV_2 |
| | Golgi | 19.uniprot.org/ | 1-12 | | Host endoplasmic | Ex{3}LL | 747-753 | Lysosome | SARS_CoV_2 |
| | | uniprotkb/P0DTC2 | | | reticulum-Golgi | | | | |
| | | | | | intermediate compartment | | | | |
| | | | | | membrane | | | | |
| | | | | | Host cell membrane | Yx{2}[VILFWCM] | 199-203, 364-368, | Lysosome | SARS_CoV_2 |
| | | | | | | | 448-452, 452-456, | | |
| | | | | | | | 488-492, 507-511, | | |
| | | | | | | | 611-615, 755-759, | | |
| | | | | | | | 836-840, 1046-1050, | | |
| | | | | | | | 1137-1141, 1208- | | |
| | | | | | | | 1212, 1214-1218 | | |
| | | | | | | GYx{2}I | 198-203 | Lysosome | SARS_CoV_2 |
| | | | | | | Kx{3}Q | 309-314 | Lysosome | SARS_CoV_2 |
| | | | | | | GYx{2}[VILFWCM] | 198-203, 1045-1050 | Lysosome | SARS_CoV_2 |
| | | | | | | Dx{1}E | 177-180, 1259-1262 | Endoplasmic | SARS_CoV_2 |
| | | | | | | | | reticulum | |
| | | | | | | [HK]x{1}K | 534-537 | Endoplasmic | SARS_CoV_2 |
| | | | | | | | | reticulum | |
| ORF3b | Golgi | \— | \— | \— | \— | \— | \— | \— | SARS_CoV_2 |
| ORF7b | Cytoplasm/ | https://covid- | \— | \— | Host membrane: Single- | Yx{2}[VILFWCM] | 9-13 | Lysosome | SARS_CoV_2 |
| | ER/PM | 19.uniprot.org/ | | | pass membrane protein | | | | |
| | | uniprotkb/P0DTC8 | | | | | | | |
| Protein | ? | \— | \— | \— | \— | Yx{2}[VILFWCM] | 4-8 | Lysosome | SARS_CoV_2 |
| 14 | | | | | | Kx{3}Q | 14-19 | Lysosome | SARS_CoV_2 |
| NSP1 | Cytoplasm/ | https://covid- | \— | \— | \— | Yx{2}[VILFWCM] | 67-71, 117-121 | Lysosome | SARS_CoV_1 |
| | PM | 19.uniprot.org/ | | | | Kx{3}Q | 10-15 | Lysosome | SARS_CoV_1 |
| | | uniprotkb/P0C6U8 | | | | Dx{1}E | 155-158 | Endoplasmic | SARS_CoV_1 |
| | | | | | | | | reticulum | |
| | | | | | | [HK]x{1}K | 44-47 | Endoplasmic | SARS_CoV_1 |
| | | | | | | | | reticulum | |
| | | | | | | Lx{2}KN | 121-126 | Golgi (early | SARS_CoV_1 |
| | | | | | | | | post -golgi | |
| | | | | | | | | comparments) | |
| NSP2 | Cytoplasm/ | https://covid- | \— | \— | \— | [DE]x{3}L[LI] | 545-551 | Lysosome\|melanosome | SARS_CoV_1 |
| | PM | 19.uniprot.org/ | | | | Ex{3}LL | 545-551 | Lysosome | SARS_CoV_1 |
| | | uniprotkb/P0C6U8 | | | | Yx{2}[VILFWCM] | 233-237, 316-320, | Lysosome | SARS_CoV_1 |
| | | | | | | | 537-541, 619-623 | | |
| | | | | | | Kx{3}Q | 481-486, 544-549, | Lysosome | SARS_CoV_1 |
| | | | | | | | 614-619 | | |
| | | | | | | Dx{1}E | 53-56, 195-198, 615- | Endoplasmic | SARS_CoV_1 |
| | | | | | | | 618 | reticulum | |
| | | | | | | [HK]x{1}K | 100-103, 110-113, | Endoplasmic | SARS_CoV_1 |
| | | | | | | | 333-336, 614-617 | reticulum | |
| NSP3 | \— | https://covid- | \— | \— | Host membrane: Multi- | [DE]x{3}L[LI] | 286-292 | Lysosome\|melanosome | SARS_CoV_1 |
| | | 19.uniprot.org/ | | | pass membrane protein | Ex{3}LL | 286-292 | Lysosome | SARS_CoV_1 |
| | | uniprotkb/P0C6U8 | | | | Yx{2}[VILFWCM] | 19-23, 104-108, 139- | Lysosome | SARS_CoV_1 |
| | | | | | | | 143, 191-195, 250- | | |
| | | | | | | | 254, 295-299, 334- | | |
| | | | | | | | 338, 343-347, 564- | | |
| | | | | | | | 568, 669-673, 694- | | |
| | | | | | | | 698, 794-798, 935- | | |
| | | | | | | | 939, 995-999, 1048- | | |
| | | | | | | | 1052, 1460-1464, | | |
| | | | | | | | 1490-1494, 1543- | | |
| | | | | | | | 1547, 1550-1554, | | |
| | | | | | | | 1556-1560, 1720- | | |
| | | | | | | | 1724, 1836-1840, | | |
| | | | | | | | 1877-1881 | | |
| | | | | | | Kx{3}Q | 377-382, 912-917, | Lysosome | SARS_CoV_1 |
| | | | | | | | 1317-1322 | | |
| | | | | | | GYx{2}[VILFWCM] | 18-23, 190-195 | Lysosome | SARS_CoV_1 |
| | | | | | | EED | 114-117, 160-163 | Nucleus | SARS_CoV_1 |
| | | | | | | SVx{5}QL | 837-846 | Peroxisomes | SARS_CoV_1 |
| | | | | | | Dx{1}E | 111-114, 117-120, | Endoplasmic | SARS_CoV_1 |
| | | | | | | | 706-709, 1821-1824 | reticulum | |
| | | | | | | SKK | 461-464 | Endoplasmic | SARS_CoV_1 |
| | | | | | | | | reticulum | |
| | | | | | | [HK]x{1}K | 224-227, 387-390, | Endoplasmic | SARS_CoV_1 |
| | | | | | | | 506-509, 563-566, | reticulum | |
| | | | | | | | 714-717, 765-768, | | |
| | | | | | | | 811-814, 814-817, | | |
| | | | | | | | 1705-1708, 1767- | | |
| | | | | | | | 1770 | | |
| | | | | | | Yx{4}LL | 834-841 | Golgi | SARS_CoV_1 |
| NSP4 | ER | https://covid- | \— | \— | Host membrane: Multi- | [DE]x{3}L[LI] | 259-265 | Lysosome\|melanosome | SARS_CoV_1 |
| | | 19.uniprot.org/ | | | pass membrane protein | Yx{2}[VILFWCM] | 25-29, 46-50, 142- | Lysosome | SARS_CoV_1 |
| | | uniprotkb/P0C6U8 | | | | | 146, 182-186, 191- | | |
| | | | | | | | 195, 248-252, 299- | | |
| | | | | | | | 303, 311-315, 335- | | |
| | | | | | | | 339, 342-346, 346- | | |
| | | | | | | | 350, 381-385, 427- | | |
| | | | | | | | 431, 444-448, 451- | | |
| | | | | | | | 455 | | |
| | | | | | | GYx{2}I | 45-50 | Lysosome | SARS_CoV_1 |
| | | | | | | GYx{2}[VILFWCM] | 45-50 | Lysosome | SARS_CoV_1 |
| | | | | | | Dx{1}E | 217-220 | Endoplasmic | SARS_CoV_1 |
| | | | | | | | | reticulum | |
| | | | | | | [HK]x{1}K | 450-453 | Endoplasmic | SARS_CoV_1 |
| | | | | | | | | reticulum | |
| NSP5 | Cytoplasm/ | \— | \— | \— | \— | Yx{2}[VILFWCM] | 54-58, 101-105, 154- | Lysosome | SARS_CoV_1 |
| | PM | | | | | | 158, 182-186, 209- | | |
| | | | | | | | 213, 239-243 | | |
| | | | | | | Kx{3}Q | 269-274 | Lysosome | SARS_CoV_1 |
| | | | | | | SPS | 121-124 | Nucleus | SARS_CoV_1 |
| | | | | | | Dx{1}E | 176-179 | Endoplasmic | SARS_CoV_1 |
| | | | | | | | | reticulum | |
| | | | | | | [HK]x{1}K | 100-103 | Endoplasmic | SARS_CoV_1 |
| | | | | | | | | reticulum | |
| | | | | | | CAAL | 265-269 | Plasma | SARS_CoV_1 |
| | | | | | | | | membrane | |
| NSP6 | ER/Golgi | https://covid- | \— | \— | Host membrane: Multi- | [DE]x{3}L[LI] | 195-201 | Lysosome\|melanosome | SARS_CoV_1 |
| | | 19.uniprot.org/ | | | pass membrane protein | Ex{3}LL | 195-201 | Lysosome | SARS_CoV_1 |
| | | uniprotkb/P0C6U8 | | | | Yx{2}[VILFWCM] | 80-84, 175-179, 196- | Lysosome | SARS_CoV_1 |
| | | | | | | | 200, 214-218, 219- | | |
| | | | | | | | 223, 224-228, 234- | | |
| | | | | | | | 238, 242-246 | | |
| | | | | | | GYx{2}[VILFWCM] | 218-223 | Lysosome | SARS_CoV_1 |
| | | | | | | [HK]x{1}K | 2-5, 61-64 | Endoplasmic | SARS_CoV_1 |
| | | | | | | | | reticulum | |
| NSP7 | Cytoplasm/ | https://covid- | \— | \— | Host cytoplasm, host | Kx{3}Q | 27-32 | Lysosome | SARS_CoV_1 |
| | PM | 19.uniprot.org/ | | | perinuclear region | | | | |
| | | uniprotkb/P0C6U8 | | | nsp7, nsp8, nsp9 and | | | | |
| | | | | | nsp10 are localized in | | | | |
| | | | | | cytoplasmic foci, largely | | | | |
| | | | | | perinuclear. Late in | | | | |
| | | | | | infection, they merge into | | | | |
| | | | | | confluent complexes | | | | |
| NSP8 | Cytoplasm/ | https://covid- | \— | \— | Host cytoplasm, host | Kx{3}Q | 61-66 | Lysosome | SARS_CoV_1 |
| | PM | 19.uniprot.org/ | | | perinuclear region | KKLKK | 36-41 | Nucleus | SARS_CoV_1 |
| | | uniprotkb/P0C6U8 | | | nsp7, nsp8, nsp9 and | Dx{1}E | 30-33 | Endoplasmic | SARS_CoV_1 |
| | | | | | nsp10 are localized in | | | reticulum | |
| | | | | | cytoplasmic foci, largely | [HK]x{1}K | 37-40 | Endoplasmic | SARS_CoV_1 |
| | | | | | perinuclear. Late in | | | reticulum | |
| | | | | | infection, they merge into | | | | |
| | | | | | confluent complexes | | | | |
| NSP9 | Cytoplasm/ | https://covid- | \— | \— | Host cytoplasm, host | Yx{2}[VILFWCM] | 66-70, 87-91 | Lysosome | SARS_CoV_1 |
| | PM | 19.uniprot.org/ | | | perinuclear region | [HK]x{1}K | 84-87 | Endoplasmic | SARS_CoV_1 |
| | | uniprotkb/P0C6U8 | | | nsp7, nsp8, nsp9 and | | | reticulum | |
| | | | | | nsp10 are localized in | | | | |
| | | | | | cytoplasmic foci, largely | | | | |
| | | | | | perinuclear. Late in | | | | |
| | | | | | infection, they merge into | | | | |
| | | | | | confluent complexes | | | | |
| NSP10 | PM/ | https://covid- | \— | \— | Host cytoplasm, host | Yx{2}[VILFWCM] | 76-80, 96-100 | Lysosome | SARS_CoV_1 |
| | Cytoplasm | 19.uniprot.org/ | | | perinuclear region | Dx{1}E | 64-67 | Endoplasmic | SARS_CoV_1 |
| | | uniprotkb/P0C6U8 | | | nsp7, nsp8, nsp9 and | | | reticulum | |
| | | | | | nsp10 are localized in | [HK]x{1}K | 93-96 | Endoplasmic | SARS_CoV_1 |
| | | | | | cytoplasmic foci, largely | | | reticulum | |
| | | | | | perinuclear. Late in | | | | |
| | | | | | infection, they merge into | | | | |
| | | | | | confluent complexes | | | | |
| NSP11 | Cytoplasm/ | \— | \— | \— | \— | \— | \— | \— | SARS_CoV_1 |
| | PM | | | | | | | | |
| NSP12 | Cytoplasm/ | \— | \— | \— | \— | [DE]x{3}L[LI] | 61-67, 465-471 | Lysosome\|melanosome | SARS_CoV_1 |
| | PM | | | | | Ex{3}LL | 61-67 | Lysosome | SARS_CoV_1 |
| | | | | | | Yx{2}[VILFWCM] | 32-36, 69-73, 87-91, | Lysosome | SARS_CoV_1 |
| | | | | | | | 149-153, 163-167, | | |
| | | | | | | | 175-179, 237-241, | | |
| | | | | | | | 479-483, 516-520, | | |
| | | | | | | | 595-599, 606-610, | | |
| | | | | | | | 619-623, 728-732, | | |
| | | | | | | | 746-750, 826-830, | | |
| | | | | | | | 877-881, 903-907, | | |
| | | | | | | | 921-925 | | |
| | | | | | | Kx{3}Q | 288-293, 871-876 | Lysosome | SARS_CoV_1 |
| | | | | | | Dx{1}E | 60-63, 608-611, 738- | Endoplasmic | SARS_CoV_1 |
| | | | | | | | 741 | reticulum | |
| | | | | | | [HK]x{1}K | 572-575 | Endoplasmic | SARS_CoV_1 |
| | | | | | | | | reticulum | |
| | | | | | | SVM | 904-907 | Plasma | SARS_CoV_1 |
| | | | | | | | | membrane | |
| | | | | | | YEDQ | 521-525 | Plasma | SARS_CoV_1 |
| | | | | | | | | membrane | |
| NSP13 | Cytoplasm/ | \— | \— | \— | \— | Yx{2}[VILFWCM] | 31-35, 224-228, 246- | Lysosome | SARS_CoV_1 |
| | PM | | | | | | 250, 253-257, 269- | | |
| | | | | | | | 273, 277-281, 306- | | |
| | | | | | | | 310, 324-328, 355- | | |
| | | | | | | | 359, 396-400, 476- | | |
| | | | | | | | 480, 541-545, 582- | | |
| | | | | | | | 586 | | |
| | | | | | | Kx{3}Q | 271-276 | Lysosome | SARS_CoV_1 |
| | | | | | | PPx{2}R | 174-179 | Nucleus | SARS_CoV_1 |
| | | | | | | Dx{1}E | 160-163 | Endoplasmic | SARS_CoV_1 |
| | | | | | | | | reticulum | |
| | | | | | | [HK]x{1}K | 345-348, 460-463, | Endoplasmic | SARS_CoV_1 |
| | | | | | | | 465-468 | reticulum | |
| NSP14 | Cytoplasm/ | \— | \— | \— | \— | Yx{2}[VILFWCM] | 50-54, 68-72, 153- | Lysosome | SARS_CoV_1 |
| | PM | | | | | | 157, 223-227, 236- | | |
| | | | | | | | 240, 295-299, 464- | | |
| | | | | | | | 468, 497-501, 510- | | |
| | | | | | | | 514, 516-520 | | |
| | | | | | | Kx{3}Q | 60-65, 338-343 | Lysosome | SARS_CoV_1 |
| | | | | | | GYx{2}[VILFWCM] | 67-72 | Lysosome | SARS_CoV_1 |
| | | | | | | Dx{1}E | 89-92, 125-128 | Endoplasmic | SARS_CoV_1 |
| | | | | | | | | reticulum | |
| | | | | | | [HK]x{1}K | 31-34, 373-376, 454- | Endoplasmic | SARS_CoV_1 |
| | | | | | | | 457 | reticulum | |
| | | | | | | YKGL | 153-157 | Golgi | SARS_CoV_1 |
| NSP15 | Cytoplasm/ | \— | \— | \— | \— | Yx{2}[VILFWCM] | 7-11, 32-36, 237-241, | Lysosome | SARS_CoV_1 |
| | PM | | | | | | 324-328, 342-346 | | |
| | | | | | | Kx{3}Q | 155-160, 204-209 | Lysosome | SARS_CoV_1 |
| | | | | | | Dx{1}E | 39-42, 199-202 | Endoplasmic | SARS_CoV_1 |
| | | | | | | | | reticulum | |
| NSP16 | Cytoplasm/ | \— | \— | \— | \— | [DE]x{3}L[LI] | 276-282 | Lysosome\|melanosome | SARS_CoV_1 |
| | PM | | | | | Yx{2}[VILFWCM] | 47-51, 181-185, 228- | Lysosome | SARS_CoV_1 |
| | | | | | | | 232, 242-246, 272- | | |
| | | | | | | | 276 | | |
| | | | | | | Kx{3}Q | 24-29, 214-219 | Lysosome | SARS_CoV_1 |
| | | | | | | [HK]x{1}K | 158-161, 214-217 | Endoplasmic | SARS_CoV_1 |
| | | | | | | | | reticulum | |
| ORF3a | Endosomes/ | https://covid- | \— | \— | Virion | Yx{2}[VILFWCM] | 73-77, 90-94, 108- | Lysosome | SARS_CoV_1 |
| | PM/ER/ | 19.uniprot.org/ | | | | | 112, 144-148, 153- | | |
| | Golgi | uniprotkb/P59632 | | | | | 157, 159-163, 199- | | |
| | | | | | | | 203, 210-214 | | |
| | | | | | Host Golgi apparatus | Kx{3}Q | 180-185 | Lysosome | SARS_CoV_1 |
| | | | | | membrane: Multi-pass | | | | |
| | | | | | membrane protein | | | | |
| | | | | | Host cell membrane: | [HK]x{1}K | 131-134, 178-181 | Endoplasmic | SARS_CoV_1 |
| | | | | | Multi-pass membrane | | | reticulum | |
| | | | | | protein | | | | |
| | | | | | Secreted | | | | SARS_CoV_1 |
| | | | | | Host cytoplasm | | | | SARS_CoV_1 |
| ORF3b | Cytoplasm | https://covid- | \— | 80-138: | Host nucleus, host | Yx{2}[VILFWCM] | 62-66 | Lysosome | SARS_CoV_1 |
| | | 19.uniprot.org/ | | Mitochondrial | nucleolus | | | | |
| | | uniprotkb/P59633 | | targeting | | | | | |
| | | | | region | | | | | |
| | | | | 134-154: | Host mitochondrion | SKK | 39-42 | Endoplasmic | SARS_CoV_1 |
| | | | | Nucleolar | | | | reticulum | |
| | | | | targeting | | | | | |
| | | | | region | | | | | |
| | | | | 135-153: | | [HK]x{1}K | 134-137 | Endoplasmic | SARS_CoV_1 |
| | | | | Bipartite | | | | reticulum | |
| | | | | nuclear | | | | | |
| | | | | localization | | | | | |
| | | | | signal | | | | | |
| ORF6 | ER/Golgi/ | https://covid- | \— | 54-63: | Host endoplasmic | Yx{2}[VILFWCM] | 48-52 | Lysosome | SARS_CoV_1 |
| | UM | 19.uniprot.org/ | | Critical | reticulum membrane | | | | |
| | | uniprotkb/P59634 | | for | Host Golgi apparatus | Dx{1}E | 52-55 | Endoplasmic | SARS_CoV_1 |
| | | | | disrupting | membrane | | | reticulum | |
| | | | | nuclear | Host cytoplasm | Lx{2}KN | 43-48 | Golgi (early | SARS_CoV_1 |
| | | | | import | | | | post -golgi | |
| | | | | | | | | comparments) | |
| | | | | | Localizes to virus-induced | | | | SARS_CoV_1 |
| | | | | | vesicular structures called | | | | |
| | | | | | double membrane vesicles | | | | |
| ORF7a | Golgi/UM | https://covid- | positions | \— | Virion | Yx{2}[VILFWCM] | 19-23, 97-101 | Lysosome | SARS_CoV_1 |
| | | 19.uniprot.org/ | 1-15 | | Host endoplasmic | KRK | 117-120 | Nucleus | SARS_CoV_1 |
| | | uniprotkb/P59635 | | | reticulum membrane: | | | | |
| | | | | | Single-pass membrane | | | | |
| | | | | | protein | | | | |
| | | | | | Host endoplasmic | [HK]x{1}K | 117-120 | Endoplasmic | SARS_CoV_1 |
| | | | | | reticulum-Golgi | | | reticulum | |
| | | | | | intermediate compartment | | | | |
| | | | | | membrane: Single-pass | | | | |
| | | | | | type I membrane protein | | | | |
| | | | | | Host Golgi apparatus | | | | SARS_CoV_1 |
| | | | | | membrane: Single-pass | | | | |
| | | | | | membrane protein | | | | |
| ORF7b | Cytoplasm/ | https://covid- | \— | \— | Host membrane: Single- | Yx{2}[VILFWCM] | 8-12 | Lysosome | SARS_CoV_1 |
| | ER/Golgi/ | 19.uniprot.org/ | | | pass membrane protein | | | | |
| | PM | uniprotkb/Q7TFA1 | | | | Dx{1}E | 34-37 | Endoplasmic | SARS_CoV_1 |
| | | | | | | | | reticulum | |
| ORF8a | ER | https://www.uniprot.org/ | \— | \— | \— | \— | \— | \— | SARS_CoV_1 |
| | | uniprot/Q19QW2 | | | | | | | |
| ORF8b | Cytoplasm/ | https://covid- | \— | \— | Host cytoplasm | \— | \— | \— | SARS_CoV_1 |
| | PM | 19.uniprot.org/ | | | Host nucleus | | | | SARS_CoV_1 |
| | | uniprotkb/O80H93 | | | | | | | |
| ORF9b | Mitochondria/ | https:/covid- | \— | 46-54: | Virion | Yx{2}[VILFWCM] | 42-46 | Lysosome | SARS_CoV_1 |
| | Cytoplasm | 19.uniprot.org/ | | nuclear | | | | | |
| | | uniprotkb/P59636 | | export | | | | | |
| | | | | signal | | | | | |
| ORF9c | Cytoplasm | \— | \— | \— | \— | Yx{2}[VILFWCM] | 4-8 | Lysosome | SARS_CoV_1 |
| | | | | | | Kx{3}Q | 14-19 | Lysosome | SARS_CoV_1 |
| M | Golgi/ER | https://covid- | \— | \— | Virion membrane: Multi- | [DE]x{3}L[LI] | 10-16, 113-119, 213- | Lysosome\|melanosome | SARS_CoV_1 |
| | | 19.uniprot.org/ | | | pass membrane protein | | 219 | | |
| | | uniprotkb/P59596 | | | Host Golgi apparatus | Ex{3}LL | 10-16, 113-119 | Lysosome | SARS_CoV_1 |
| | | | | | membrane: Multi-pass | | | | |
| | | | | | membrane protein | | | | |
| | | | | | | Yx{2}[VILFWCM] | 176-180 | Lysosome | SARS_CoV_1 |
| E | Golgi/ER | https://covid- | \— | \— | Host cytoplasmic vesicle | [DE]x{3}L[LI] | 9-13 | Lysosome\|melanosome | SARS_CoV_1 |
| | | 19.uniprot.org/ | | | membrane: Peripheral | | | | |
| | | uniprotkb/P59637 | | | membrane protein | | | | |
| | | | | | Host cytoplasm | Yx{2}[VILFWCM] | 1-5, 58-62 | Lysosome | SARS_CoV_1 |
| | | | | | Host endoplasmic | | | | SARS_CoV_1 |
| | | | | | reticulum | | | | |
| | | | | | Host nucleus | | | | SARS_CoV_1 |
| | | | | | Host mitochondrion | | | | SARS_CoV_1 |
| | | | | | Host endoplasmic | | | | SARS_CoV_1 |
| | | | | | reticulum-Golgi | | | | |
| | | | | | intermediate compartment | | | | |
| | | | | | Host Golgi apparatus | | | | SARS_CoV_1 |
| | | | | | membrane | | | | |
| N | Cytoplasm/ | https://covid- | \— | \— | Virion | [DE]x{3}L[LI] | 348-354 | Lysosome\|melanosome | SARS_CoV_1 |
| | PM | 19.uniprot.org/ | | | Host endoplasmic | Yx{2}[VILFWCM] | 298-302, 360-364 | Lysosome | SARS_CoV_1 |
| | | uniprotkb/P59595 | | | reticulum-Golgi | | | | |
| | | | | | intermediate compartment | | | | |
| | | | | | Host Golgi apparatus | Kx{3}Q | 237-242, 256-261, | Lysosome | SARS_CoV_1 |
| | | | | | | | 299-304 | | |
| | | | | | Host cytoplasm, host | SKK | 255-258 | Endoplasmic | SARS_CoV_1 |
| | | | | | perinuclear region | | | reticulum | |
| | | | | | Located inside the virion, | [HK]x{1}K | 59-62, 100-103, 370- | Endoplasmic | SARS_CoV_1 |
| | | | | | complexed with the viral | | 373, 373-376 | reticulum | |
| | | | | | RNA. Probably associates | | | | |
| | | | | | with ER-derived | | | | |
| | | | | | membranes where it | | | | |
| | | | | | participates in viral RNA | | | | |
| | | | | | synthesis and virus | | | | |
| | | | | | budding | | | | |
| S | PM/ER/ | https://covid- | positions | \— | Virion membrane | [DE]x{3}L[LI] | 729-735 | Lysosome\|melanosome | SARS_CoV_1 |
| | Golgi | 19.uniprot.org/ | 1-13 | | Host endoplasmic | Ex{3}LL | 729-735 | Lysosome | SARS_CoV_1 |
| | | uniprotkb/P59594 | | | reticulum-Golgi | | | | |
| | | | | | intermediate compartment | | | | |
| | | | | | membrane | | | | |
| | | | | | Host cell membrane | Yx{2}[VILFWCM] | 62-66, 199-203, 351- | Lysosome | SARS_CoV_1 |
| | | | | | | | 355, 439-443, 474- | | |
| | | | | | | | 478, 493-497, 597- | | |
| | | | | | | | 601, 659-663, 737- | | |
| | | | | | | | 741, 818-822, 1028- | | |
| | | | | | | | 1032, 1119-1123, | | |
| | | | | | | | 1190-1194, 1196- | | |
| | | | | | | | 1200 | | |
| | | | | | | GYx{2}I | 198-203 | Lysosome | SARS_CoV_1 |
| | | | | | | Kx{3}Q | 296-301, 910-915 | Lysosome | SARS_CoV_1 |
| | | | | | | GYx{2}[VILFWCM] | 198-203, 1027-1032 | Lysosome | SARS_CoV_1 |
| | | | | | | Dx{1}E | 1241-1244 | Endoplasmic | SARS_CoV_1 |
| | | | | | | | | reticulum | |
| | | | | | | [HK]x{1}K | 187-190, 444-447 | Endoplasmic | SARS_CoV_1 |
| | | | | | | | | reticulum | |
| | | | | | | Yx{4}LL | 659-666 | Golgi | SARS_CoV_1 |
| NSP1 | Cytoplasm | https:/www.uniprot.org/ | \— | \— | \— | Yx{2}[VILFWCM] | 55-59, 70-74, 154- | Lysosome | MERS |
| | | uniprot/K9N7C7 | | | | | 158 | | |
| | | | | | | Dx{1}E | 50-53, 132-135, 172- | Endoplasmic | MERS |
| | | | | | | | 175 | reticulum | |
| | | | | | | [HK]x{1}K | 178-181 | Endoplasmic | MERS |
| | | | | | | | | reticulum | |
| NSP2 | Cytoplasm/ | https:/www.uniprot.org/ | \— | \— | \— | Yx{2}[VILFWCM] | 20-24, 56-60, 93-97, | Lysosome | MERS |
| | PM | uniprot/K9N7C7 | | | | | 238-242, 359-363, | | |
| | | | | | | | 366-370, 384-388, | | |
| | | | | | | | 403-407, 433-437, | | |
| | | | | | | | 552-556, 622-626, | | |
| | | | | | | | 642-646 | | |
| | | | | | | Kx{3}Q | 560-565 | Lysosome | MERS |
| | | | | | | EED | 635-638 | Nucleus | MERS |
| | | | | | | Dx{1}E | 34-37, 44-47, 174- | Endoplasmic | MERS |
| | | | | | | | 177 | reticulum | |
| | | | | | | SKK | 575-578 | Endoplasmic | MERS |
| | | | | | | | | reticulum | |
| | | | | | | [HK]x{1}K | 118-121, 524-527, | Endoplasmic | MERS |
| | | | | | | | 558-561 | reticulum | |
| NSP3 | ER | https:/www.uniprot.org/ | \— | \— | Host membrane; Multi- | [DE]x{3}L[LI] | 775-781, 793-799, | Lysosome\|melanosome | MERS |
| | | uniprot/K9N7C7 | | | pass membrane protein | | 1044-1050, 1522- | | |
| | | | | | | | 1528, 1808-1814 | | |
| | | | | | Host cytoplasm | Yx{2}[VILFWCM] | 367-371, 373-377, | Lysosome | MERS |
| | | | | | | | 415-419, 431-435, | | |
| | | | | | | | 530-534, 566-570, | | |
| | | | | | | | 700-704, 783-787, | | |
| | | | | | | | 837-841, 1037-1041, | | |
| | | | | | | | 1055-1059, 1175- | | |
| | | | | | | | 1179, 1364-1368, | | |
| | | | | | | | 1370-1374, 1415- | | |
| | | | | | | | 1419, 1500-1504, | | |
| | | | | | | | 1513-1517, 1629- | | |
| | | | | | | | 1633, 1658-1662, | | |
| | | | | | | | 1681-1685, 1839- | | |
| | | | | | | | 1843 | | |
| | | | | | | Kx{3}Q | 312-317, 326-331, | Lysosome | MERS |
| | | | | | | | 1781-1786 | | |
| | | | | | | GYx{2}[VILFWCM] | 565-570 | Lysosome | MERS |
| | | | | | | Dx{1}E | 114-117, 124-127, | Endoplasmic | MERS |
| | | | | | | | 149-152, 235-238, | reticulum | |
| | | | | | | | 1766-1769 | | |
| | | | | | | [HK]x{1}K | 245-248, 296-299, | Endoplasmic | MERS |
| | | | | | | | 440-443, 767-770, | reticulum | |
| | | | | | | | 932-935, 1066-1069, | | |
| | | | | | | | 1133-1136, 1211- | | |
| | | | | | | | 1214, 1642-1645 | | |
| | | | | | | Lx{2}KN | 648-653 | Golgi (early | MERS |
| | | | | | | | | post -golgi | |
| | | | | | | | | comparments) | |
| NSP3_C740A | ER | \— | \— | \— | \— | [DE]x{3}L[LI] | 775-781, 793-799, | Lysosome\|melanosome | MERS |
| | | | | | | | 1044-1050, 1522- | | |
| | | | | | | | 1528, 1808-1814 | | |
| | | | | | | Yx{2}[VILFWCM] | 367-371, 373-377, | Lysosome | MERS |
| | | | | | | | 415-419, 431-435, | | |
| | | | | | | | 530-534, 566-570, | | |
| | | | | | | | 700-704, 783-787, | | |
| | | | | | | | 837-841, 1037-1041, | | |
| | | | | | | | 1055-1059, 1175- | | |
| | | | | | | | 1179, 1364-1368, | | |
| | | | | | | | 1370-1374, 1415- | | |
| | | | | | | | 1419, 1500-1504, | | |
| | | | | | | | 1513-1517, 1629- | | |
| | | | | | | | 1633, 1658-1662, | | |
| | | | | | | | 1681-1685, 1839- | | |
| | | | | | | | 1843 | | |
| | | | | | | Kx{3}Q | 312-317, 326-331, | Lysosome | MERS |
| | | | | | | | 1781-1786 | | |
| | | | | | | GYx{2}[VILFWCM] | 565-570 | Lysosome | MERS |
| | | | | | | Dx{1}E | 114-117, 124-127, | Endoplasmic | MERS |
| | | | | | | | 149-152, 235-238, | reticulum | |
| | | | | | | | 1766-1769 | | |
| | | | | | | [HK]x{1}K | 245-248, 296-299, | Endoplasmic | MERS |
| | | | | | | | 440-443, 767-770, | reticulum | |
| | | | | | | | 932-935, 1066-1069, | | |
| | | | | | | | 1133-1136, 1211- | | |
| | | | | | | | 1214, 1642-1645 | | |
| | | | | | | Lx{2}KN | 648-653 | Golgi (early | MERS |
| | | | | | | | | post -golgi | |
| | | | | | | | | comparments) | |
| NSP4 | ER | https:/www.uniprot.org/ | \— | \— | Host membrane; Multi- | Yx{2}[VILFWCM] | 31-35, 140-144, 148- | Lysosome | MERS |
| | | uniprot/K9N7C7 | | | pass membrane protein | | 152, 188-192, 227- | | |
| | | | | | | | 231, 284-288, 318- | | |
| | | | | | | | 322, 349-353, 355- | | |
| | | | | | | | 359, 373-377, 436- | | |
| | | | | | | | 440, 448-452, 458- | | |
| | | | | | | | 462 | | |
| | | | | | Host cytoplasm | Dx{1}E | 167-170 | Endoplasmic | MERS |
| | | | | | | | | reticulum | |
| | | | | | | SKK | 405-408 | Endoplasmic | MERS |
| | | | | | | | | reticulum | |
| | | | | | | [HK]x{1}K | 310-313, 457-460 | Endoplasmic | MERS |
| | | | | | | | | reticulum | |
| NSP5 | PM/ | \— | \— | \— | \— | Yx{2}[VILFWCM] | 54-58, 185-189, 202- | Lysosome | MERS |
| | Cytoplasm | | | | | | 206, 212-216, 273- | | |
| | | | | | | | 277 | | |
| | | | | | | Kx{3}Q | 191-196 | Lysosome | MERS |
| | | | | | | Dx{1}E | Dec-15 | Endoplasmic | MERS |
| | | | | | | | | reticulum | |
| NSP5_C148A | Cytoplasm/ | \— | \— | \— | \— | Yx{2}[VILFWCM] | 54-58, 185-189, 202- | Lysosome | MERS |
| | PM | | | | | | 206, 212-216, 273- | | |
| | | | | | | | 277 | | |
| | | | | | | Kx{3}Q | 191-196 | Lysosome | MERS |
| | | | | | | Dx{1}E | Dec-15 | Endoplasmic | MERS |
| | | | | | | | | reticulum | |
| NSP6 | ER/Golgi | https:/www.uniprot.org/ | \— | \— | Host membrane; Multi- | Yx{2}[VILFWCM] | 22-26, 80-84, 119- | Lysosome | MERS |
| | | uniprot/K9N7C7 | | | pass membrane protein | | 123, 166-170, 193- | | |
| | | | | | | | 197, 216-220, 226- | | |
| | | | | | | | 230 | | |
| | | | | | | Kx{3}Q | 247-252 | Lysosome | MERS |
| | | | | | | [HK]x{1}K | 61-64 | Endoplasmic | MERS |
| | | | | | | | | reticulum | |
| NSP7 | Cytoplasm/ | https:/www.uniprot.org/ | \— | \— | host perinuclear region | \— | \— | \— | MERS |
| | PM | uniprot/K9N7C7 | | | Note: nsp7, nsp8, nsp9 and | | | | |
| | | | | | nsp10 are localized in | | | | |
| | | | | | cytoplasmic foci, largely | | | | |
| | | | | | perinuclear. Late in | | | | |
| | | | | | infection, they merge into | | | | |
| | | | | | confluent complexes | | | | |
| NSP8 | Cytoplasm/ | https:/www.uniprot.org/ | \— | \— | host perinuclear region | Yx{2}[VILFWCM] | 145-149 | Lysosome | MERS |
| | PM | uniprot/K9N7C7 | | | Note: nsp7, nsp8, nsp9 and | Dx{1}E | 164-167 | Endoplasmic | MERS |
| | | | | | nsp10 are localized in | | | reticulum | |
| | | | | | cytoplasmic foci, largely | [HK]x{1}K | 52-55, 81-84 | Endoplasmic | MERS |
| | | | | | perinuclear. Late in | | | reticulum | |
| | | | | | infection, they merge into | | | | |
| | | | | | confluent complexes | | | | |
| NSP9 | Cytoplasm/ | https:/www.uniprot.org/ | \— | \— | host perinuclear region | Yx{2}[VILFWCM] | 31-35, 49-53, 84-88 | Lysosome | MERS |
| | PM | uniprot/K9N7C7 | | | Note: nsp7, nsp8, nsp9 and | | | | |
| | | | | | nsp10 are localized in | | | | |
| | | | | | cytoplasmic foci, largely | | | | |
| | | | | | perinuclear. Late in | | | | |
| | | | | | infection, they merge into | | | | |
| | | | | | confluent complexes | | | | |
| NSP10 | Cytoplasm/ | https:/www.uniprot.org/ | \— | \— | host perinuclear region | Yx{2}[VILFWCM] | 27-31 | Lysosome | MERS |
| | PM | uniprot/K9N7C7 | | | Note: nsp7, nsp8, nsp9 and | Dx{1}E | 64-67 | Endoplasmic | MERS |
| | | | | | nsp10 are localized in | | | reticulum | |
| | | | | | cytoplasmic foci, largely | [HK]x{1}K | 91-94 | Endoplasmic | MERS |
| | | | | | perinuclear. Late in | | | reticulum | |
| | | | | | infection, they merge into | | | | |
| | | | | | confluent complexes | | | | |
| NSP11 | Cytoplasm/ | \— | \— | \— | \— | [DE]x{3}L[LI] | Sep-15 | Lysosome\|melanosome | MERS |
| | PM | | | | | Ex{3}LL | Sep-15 | Lysosome | MERS |
| NSP12 | Golgi/ | https:/www.uniprot.org/ | \— | \— | \— | Yx{2}[VILFWCM] | 71-75, 89-93, 124- | Lysosome | MERS |
| | Cytoplasm | uniprot/K9N7C7 | | | | | 128, 150-154, 176- | | |
| | | | | | | | 180, 239-243, 349- | | |
| | | | | | | | 353, 421-425, 480- | | |
| | | | | | | | 484, 517-521, 596- | | |
| | | | | | | | 600, 607-611, 620- | | |
| | | | | | | | 624, 667-671, 729- | | |
| | | | | | | | 733, 746-750, 878- | | |
| | | | | | | | 882, 893-897, 904- | | |
| | | | | | | | 908, 922-926 | | |
| | | | | | | Kx{3}Q | 289-294 | Lysosome | MERS |
| | | | | | | Dx{1}E | 718-721, 875-878 | Endoplasmic | MERS |
| | | | | | | | | reticulum | |
| | | | | | | [HK]x{1}K | 41-44, 110-113, 348- | Endoplasmic | MERS |
| | | | | | | | 351, 573-576 | reticulum | |
| | | | | | | SVM | 905-908 | Plasma | MERS |
| | | | | | | | | membrane | |
| NSP13 | Mitochondria/ | https:/www.uniprot.org/ | \— | \— | \— | [DE]x{3}L[LI] | 160-166 | Lysosome\|melanosome | MERS |
| | PM | uniprot/K9N7C7 | | | | Ex{3}LL | 160-166 | Lysosome | MERS |
| | | | | | | Yx{2}[VILFWCM] | 31-35, 70-74, 93-97, | Lysosome | MERS |
| | | | | | | | 246-250, 253-257, | | |
| | | | | | | | 277-281, 306-310, | | |
| | | | | | | | 324-328, 343-347, | | |
| | | | | | | | 541-545 | | |
| | | | | | | PPx{2}R | 174-179 | Nucleus | MERS |
| | | | | | | SPS | 100-103 | Nucleus | MERS |
| | | | | | | [HK]x{1}K | 171-174, 392-395 | Endoplasmic | MERS |
| | | | | | | | | reticulum | |
| NSP14 | Cytoplasm/ | https:/www.uniprot.org/ | \— | \— | \— | Yx{2}[VILFWCM] | 26-30, 51-55, 69-73, | Lysosome | MERS |
| | PM | uniprot/K9N7C7 | | | | | 180-184, 224-228, | | |
| | | | | | | | 233-237, 237-241, | | |
| | | | | | | | 260-264, 296-300, | | |
| | | | | | | | 462-466, 495-499, | | |
| | | | | | | | 508-512, 514-518 | | |
| | | | | | | GYx{2}[VILFWCM] | 68-73, 232-237 | Lysosome | MERS |
| | | | | | | Dx{1}E | 90-93, 126-129, 293- | Endoplasmic | MERS |
| | | | | | | | 296 | reticulum | |
| | | | | | | [HK]x{1}K | 32-35, 301-304 | Endoplasmic | MERS |
| | | | | | | | | reticulum | |
| NSP15 | Cytoplasm/ | https:/www.uniprot.org/ | \— | \— | \— | Yx{2}[VILFWCM] | 81-85, 104-108, 145- | Lysosome | MERS |
| | PM | uniprot/K9N7C7 | | | | | 149, 153-157, 176- | | |
| | | | | | | | 180, 234-238, 339- | | |
| | | | | | | | 343 | | |
| | | | | | | Dx{1}E | 87-90, 205-208 | Endoplasmic | MERS |
| | | | | | | | | reticulum | |
| | | | | | | [HK]x{1}K | 141-144 | Endoplasmic | MERS |
| | | | | | | | | reticulum | |
| NSP16 | Cytoplasm/ | https:/www.uniprot.org/ | \— | \— | \— | Yx{2}[VILFWCM] | 47-51, 181-185, 228- | Lysosome | MERS |
| | PM | uniprot/K9N7C7 | | | | | 232, 242-246, 299- | | |
| | | | | | | | 303 | | |
| | | | | | | [HK]x{1}K | 253-256 | Endoplasmic | MERS |
| | | | | | | | | reticulum | |
| E | Golgi/ER | \— | \— | \— | \— | Yx{2}[VILFWCM] | 65-69 | Lysosome | MERS |
| M | Golgi/ER | https://www.uniprot.org/ | \— | \— | Virion membrane | [DE]x{3}L[LI] | 113-119 | Lysosome | MERS |
| | | uniprot/K9N7A1 | | | Host Golgi apparatus | Ex{3}LL | 113-119 | Lysosome | MERS |
| | | | | | membrane | | | | |
| | | | | | | Yx{2}[VILFWCM] | 159-163 | Lysosome | MERS |
| | | | | | | Dx{1}E | 210-213 | Endoplasmic | MERS |
| | | | | | | | | reticulum | |
| | | | | | | [HK]x{1}K | 146-149 | Endoplasmic | MERS |
| | | | | | | | | reticulum | |
| N | Cytoplasm/ | \— | \— | \— | \— | Yx{2}[VILFWCM] | 43-47, 214-218, 343- | Lysosome | MERS |
| | PM | | | | | | 347, 357-361 | | |
| | | | | | | Kx{3}Q | 312-317, 363-368 | Lysosome | MERS |
| | | | | | | [HK]x{1}K | 49-52, 228-231, 246- | Endoplasmic | MERS |
| | | | | | | | 249, 363-366, 366- | reticulum | |
| | | | | | | | 369 | | |
| | | | | | | Lx{2}KN | 336-341 | Golgi (early | MERS |
| | | | | | | | | post -golgi | |
| | | | | | | | | comparments) | |
| S | PM/ER/ | https://www.uniprot.org/ | \— | \— | Virion membrane; Single- | [DE]x{3}L[LI] | 383-389, 991-997 | Lysosome\|melanosome | MERS |
| | Golgi | uniprot/K9N5Q8 | | | pass type I membrane | | | | |
| | | | | | protein | | | | |
| | | | | | Host endoplasmic | Yx{2}[VILFWCM] | 17-21, 63-67, 70-74, | Lysosome | MERS |
| | | | | | reticulum-Golgi | | 143-147, 183-187, | | |
| | | | | | intermediate compartment | | 200-204, 230-234, | | |
| | | | | | membrane UniRule | | 269-273, 286-290, | | |
| | | | | | annotation; Single-pass | | 291-295, 350-354, | | |
| | | | | | type I membrane protein | | 437-441, 496-500, | | |
| | | | | | UniRule annotation | | 522-526, 634-638, | | |
| | | | | | | | 647-651, 703-707, | | |
| | | | | | | | 776-780, 823-827, | | |
| | | | | | | | 908-912, 931-935, | | |
| | | | | | | | 1152-1156, 1210- | | |
| | | | | | | | 1214, 1263-1267, | | |
| | | | | | | | 1279-1283, 1291- | | |
| | | | | | | | 1295, 1297-1301 | | |
| | | | | | Host cell membrane | Kx{3}Q | 594-599 | Lysosome | MERS |
| | | | | | UniRule annotation; | | | | |
| | | | | | Single-pass type I | | | | |
| | | | | | membrane protein UniRule | | | | |
| | | | | | annotation | | | | |
| | | | | | | YPAF | 143-147 | Lysosome | MERS |
| | | | | | | GYx{2}[VILFWCM] | 907-912, 930-935 | Lysosome | MERS |
| | | | | | | SPS | 132-135 | Nucleus | MERS |
| | | | | | | Dx{1}E | 354-357, 663-666, | Endoplasmic | MERS |
| | | | | | | | 1343-1346 | reticulum | |
| | | | | | | [HK]x{1}K | 1099-1102, 1329- | Endoplasmic | MERS |
| | | | | | | | 1332 | reticulum | |
| | | | | | | Yx{4}LL | 408-415 | Golgi | MERS |
| ORF3 | Golgi/ER | https://www.uniprot.org/ | positions | \— | Host endoplasmic | Dx{1}E | 75-78 | Endoplasmic | MERS |
| | | uniprot/K9N796 | 1-23 | | reticulum | | | reticulum | |
| | | | | | | Yx{2}[VILFWCM] | 34-38, 54-58 | Lysosome | MERS |
| ORF4a | Cytoplasm/ | https://www.uniprot.org/ | \— | \— | Host cytoplasm | [DE]x{3}L[LI] | 1-7 | Lysosome\|melanosome | MERS |
| | PM | uniprot/K9N54V0 | | | | YTPL | 31-35 | Lysosome | MERS |
| | | | | | | Yx{2}[VILFWCM] | 2-6, 18-22, 31-35 | Lysosome | MERS |
| ORF4b | Cytoplasm | https://www.uniprot.org/ | \— | 22-38: | Host nucleus | Yx{2}[VILFWCM] | 55-59, 237-241 | Lysosome | MERS |
| | | uniprot/K9N643 | | Nuclear | host nucleolus | | | | MERS |
| | | | | localization | host cytoplasm | | | | MERS |
| | | | | motif | | | | | |
| ORF5 | Golgi/ | https://www.uniprot.org/ | \— | \— | host membrane | Yx{2}[VILFWCM] | 71-75, 76-80, 121- | Lysosome | MERS |
| | ER/UM | uniprot/K9N7D2 | | | | | 125, 173-177 | | |
| | | | | | host Golgi apparatus | [HK]x{1}K | 147-150 | Endoplasmic | MERS |
| | | | | | | | | reticulum | |
| ORF8b | ER/UM | https://www.uniprot.org/ | \— | \— | \— | \— | \— | \— | MERS |
| | | uniprot/A0A2D0Y3F8 | | | | | | | |

The localization of our Strep-tagged constructs to sequence based predicted localization was compared, and found to generally agree with the observed localization of the individually expressed proteins (FIG. 6E and Table 6A-D provided in U.S. Provisional Application No. 63/091,929 filed on Oct. 15, 2020, expressly incorporated by reference herein). This agreement suggests that sequence elements may target the proteins to each cellular compartment. Most orthologous proteins show the same localization across the viruses (FIG. 6B). Moreover, changes in localization, as observed for some viral proteins across strains, do not coincide with strong changes in viral-host protein interactions (FIG. 6F). Overall, these results suggest that changes in protein localization are unlikely to be a major source of differences in host targeting mechanisms.

Referring to FIG. 6E, the localization of all coronavirus proteins as predicted based on a machine learning algorithm or determined experimentally for Strep-tagged construct is shown.

Referring to FIG. 6F, the prey overlap per bait measured as Jaccard index comparing SARS-CoV-2 vs. SARS-CoV-1 (red dots) and SARS-CoV-2 vs. MERS-CoV (blue dots) for all viral baits (All), viral baits found in the same cellular compartment (Yes) and viral baits found in different compartments (No), when comparing predicted vs. experimental localization is shown.

Comparison of Host Targeted Processes Identifies Conserved Mechanisms with Divergent Implementations

To study the conservation of targeted host factors and processes, a clustering approach was first used to compare the overlap in protein interactions for the three viruses (FIG. 2A). 7 clusters of viral-host interactions corresponding to those that are specific to each or shared among the viruses were defined. The largest pairwise overlap was observed between SARS-CoV-1 and SARS-CoV-2 (FIG. 2A), as expected from their closer evolutionary relationship. A functional enrichment analysis (FIG. 2B and Table 9 Å-J) highlighted host processes that are targeted through interactions conserved across all three viruses including ribosome biogenesis and regulation of RNA metabolism. Conserved interactions between SARS-CoV-1 and SARS-CoV-2, but not MERS-CoV, were enriched in endosomal and Golgi vesicle transport (FIG. 2B). Despite the small fraction (7.1%) of interactions conserved between SARS-CoV-1 and MERS-CoV, but not SARS-CoV-2, these were strongly enriched in translation initiation and myosin complex proteins (FIG. 2B).

Referring to FIG. 2B, GO enrichment analysis of each cluster from FIG. 2B is shown, with the top six most significant terms per cluster. Color indicates −log 10(q) and number of genes with significant (q<0.05; white) or non-significant enrichment (q>0.05; grey) is shown.

TABLE 9A
CLUSTER 1

| Description | GeneRatio | BgRatio | pvalue | p.adjust | geneID |
|---|---|---|---|---|---|
| GO_EUKARYOTIC_ | 10/36 | 15/18046 | 7.53E−25 | 5.09E−22 | 8665/8667/ |
| 48S_PREINITIATION_ | | | | | 8666/8669/ |
| COMPLEX | | | | | 3646/8661/ |
| | | | | | 10480/8663/ |
| | | | | | 27335/51386 |
| GO_EUKARYOTIC_ | 10/36 | 16/18046 | 2.01E−24 | 5.09E−22 | 8665/8667/ |
| TRANSLATION_ | | | | | 8666/8669/ |
| INITIATION_FACTOR_3_ | | | | | 3646/8661/ |
| COMPLEX | | | | | 10480/8663/ |
| | | | | | 27335/ |
| | | | | | 51386 |
| GO_FORMATION_ | 10/36 | 16/18046 | 2.01E−24 | 5.09E−22 | 8665/8667/ |
| OF_CYTOPLASMIC_ | | | | | 8666/8669/ |
| TRANSLATION_ | | | | | 3646/8661/ |
| INITIATION_ | | | | | 10480/ |
| COMPLEX | | | | | 8663/ |
| | | | | | 27335/ |
| | | | | | 51386 |
| GO_TRANSLATION_ | 10/36 | 18/18046 | 1.09E−23 | 2.08E−21 | 8665/8667/ |
| PREINITIATION_ | | | | | 8666/8669/ |
| COMPLEX | | | | | 3646/8661/ |
| | | | | | 10480/ |
| | | | | | 8663/ |
| | | | | | 27335/ |
| | | | | | 51386 |
| GO_CYTOPLASMIC_ | 10/36 | 31/18046 | 1.09E−20 | 1.66E−18 | 8665/8667/ |
| TRANSLATIONAL_ | | | | | 8666/8669/ |
| INITIATION | | | | | 3646/8661/ |
| | | | | | 10480/ |
| | | | | | 8663/ |
| | | | | | 27335/ |
| | | | | | 51386 |
| GO_TRANSLATION_ | 10/36 | 51/18046 | 3.06E−18 | 3.88E−16 | 8665/8667/ |
| INITIATION_ | | | | | 8666/8669/ |
| FACTOR_ACTIVITY | | | | | 3646/8661/ |
| | | | | | 10480/ |
| | | | | | 8663/ |
| | | | | | 27335/ |
| | | | | | 51386 |
| GO_TRANSLATION_ | 11/36 | 85/18046 | 7.07E−18 | 7.68E−16 | 10985/8665/ |
| FACTOR_ | | | | | 8667/8666/ |
| ACTIVITY_RNA_ | | | | | 8669/3646/ |
| BINDING | | | | | 8661/ |
| | | | | | 10480/ |
| | | | | | 8663/ |
| | | | | | 27335/ |
| | | | | | 51386 |
| GO_TRANSLATION_ | 11/36 | 109/18046 | 1.23E−16 | 1.17E−14 | 10985/8 |
| REGULATOR_ | | | | | 665/8667/ |
| ACTIVITY_ | | | | | 8666/ |
| NUCLEIC_ACID_ | | | | | 8669/ |
| BINDING | | | | | 3646/ |
| | | | | | 8661/ |
| | | | | | 10480/ |
| | | | | | 8663/ |
| | | | | | 27335/ |
| | | | | | 51386 |
| GO_RIBO- | 15/36 | 419/18046 | 8.55E−16 | 7.23E−14 | 55127/8665/ |
| NUCLEOPROTEIN_ | | | | | 8667/8666/ |
| COMPLEX_ | | | | | 8669/3646/ |
| BIOGENESIS | | | | | 8661/ |
| | | | | | 10480/ |
| | | | | | 8663/27335/ |
| | | | | | 51386/ |
| | | | | | 4931/9816/ |
| | | | | | 5822/57647 |
| GO_TRANSLATION_ | 11/36 | 140/18046 | 2.09E−15 | 1.59E−13 | 10985/8665/ |
| REGULATOR_ACTIVITY | | | | | 8667/8666/ |
| | | | | | 8669/3646/ |
| | | | | | 8661/10480/ |
| | | | | | 8663/27335/ |
| | | | | | 51386 |
| GO_CYTOPLASMIC_ | 10/36 | 99/18046 | 3.50E−15 | 2.42E−13 | 8665/8667/866 |
| TRANSLATION | | | | | 6/8669/3646/ |
| | | | | | 8661/10480/ |
| | | | | | 8663/ |
| | | | | | 27335/51386 |
| GO_RIBONUCLEOPROTEIN_ | 11/36 | 193/18046 | 7.48E−14 | 4.75E−12 | 8665/8667/ |
| COMPLEX_ | | | | | 8666/8669/ |
| SUBUNIT_ORGANIZATION | | | | | 3646/8661/ |
| | | | | | 10480/8663/ |
| | | | | | 27335/51386/ |
| | | | | | 5822 |
| GO_TRANSLATIONAL_ | 10/36 | 192/18046 | 2.94E−12 | 1.72E−10 | 8665/8667/ |
| INITIATION | | | | | 8666/8669/ |
| | | | | | 3646/8661/ |
| | | | | | 10480/8663/ |
| | | | | | 27335/51386 |
| GO_ACTIN_FILAMENT_ | 8/36 | 190/18046 | 3.06E−09 | 1.67E−07 | 7168/7111/ |
| BINDING | | | | | 7171/2314/ |
| | | | | | 79784/3 |
| | | | | | 99687/4646/ |
| | | | | | 4644 |
| GO_ACTIN_FILAMENT_ | 7/36 | 143/18046 | 1.17E−08 | 5.92E−07 | 7168/140465/ |
| BASED_MOVEMENT | | | | | 7111/ |
| | | | | | 7171/79784/ |
| | | | | | 4646/4644 |
| GO_VIRAL_TRANSLATION | 4/36 | 15/18046 | 1.79E−08 | 8.52E−07 | 8665/8666/ |
| | | | | | 8661/51386 |
| GO_MYOSIN_COMPLEX | 5/36 | 55/18046 | 7.66E−08 | 3.43E−06 | 140465/79784/ |
| | | | | | 399687/4646/ |
| | | | | | 4644 |
| GO_ACTOMYOSIN | 5/36 | 79/18046 | 4.79E−07 | 2.03E−05 | 7168/7171/ |
| | | | | | 79784/ |
| | | | | | 399687/4644 |
| GO_UNCONVENTIONAL_ | 3/36 | 10/18046 | 8.67E−07 | 3.47E−05 | 140465/4646/ |
| MYOSIN_COMPLEX | | | | | 4644 |
| GO_MUSCLE_FILAMENT_ | 4/36 | 39/18046 | 1.04E−06 | 3.97E−05 | 7168/140465/ |
| SLIDING | | | | | 7111/7171 |
| GO_ACTIN_BINDING | 8/36 | 428/18046 | 1.58E−06 | 5.74E−05 | 7168/7111/ |
| | | | | | 7171/2314/ |
| | | | | | 79784/399687/ |
| | | | | | 4646/4644 |
| GO_ACTIN_FILAMENT | 5/36 | 119/18046 | 3.67E−06 | 0.000126891 | 7168/7111/ |
| | | | | | 7171/4646/ |
| | | | | | 4644 |
| GO_MICROFILAMENT_ | 3/36 | 22/18046 | 1.09E−05 | 0.000361938 | 79784/4646/ |
| MOTOR_ACTIVITY | | | | | 4644 |
| GO_MYOFILAMENT | 3/36 | 27/18046 | 2.06E−05 | 0.000654302 | 7168/7111/ |
| | | | | | 7171 |
| GO_TRANSLATION_ | 3/36 | 32/18046 | 3.48E−05 | 0.001057861 | 8665/10480/ |
| INITIATION_ | | | | | 8663 |
| FACTOR_BINDING | | | | | |
| GO_MATURATION_OF_ | 3/36 | 35/18046 | 4.57E−05 | 0.001336711 | 55127/5822/ |
| SSU_RRNA_FROM_ | | | | | 57647 |
| TRICISTRONIC_RRNA_ | | | | | |
| TRANSCRIPT_SSU_RRNA_ | | | | | |
| 5_8S_RRNA_LSU_RRNA | | | | | |
| GO_MUSCLE_CONTRACTION | 6/36 | 362/18046 | 7.32E−05 | 0.0020634 | 8106/7168/ |
| | | | | | 140465/7111/ |
| | | | | | 7171/79784 |
| GO_STRUCTURAL_ | 3/36 | 43/18046 | 8.52E−05 | 0.002314901 | 7168/ |
| CONSTITUENT_ | | | | | 140465/7171 |
| OF_MUSCLE | | | | | |
| GO_ACTIN_MEDIATED_ | 4/36 | 121/18046 | 9.60E−05 | 0.002468609 | 7168/140465/ |
| CELL_CONTRACTION | | | | | 7111/ |
| | | | | | 7171 |
| GO_CONTRACTILE_FIBER | 5/36 | 235/18046 | 9.73E−05 | 0.002468609 | 5663/7168/ |
| | | | | | 140465/ |
| | | | | | 7111/7171 |
| GO_MATURATION_ | 3/36 | 47/18046 | 0.000111299 | 0.002732219 | 55127/ |
| OF_SSU_RRNA | | | | | 5822/57647 |
| GO_MOTOR_ACTIVITY | 4/36 | 136/18046 | 0.000150757 | 0.003585197 | 140465/ |
| | | | | | 79784/ |
| | | | | | 4646/4644 |
| GO_IRES_DEPENDENT_ | 2/36 | 10/18046 | 0.000172377 | 0.003858207 | 8665/8661 |
| VIRAL_ | | | | | |
| TRANSLATIONAL_ | | | | | |
| INITIATION | | | | | |
| GO_REGULATION_ | 2/36 | 10/18046 | 0.000172377 | 0.003858207 | 3646/8663 |
| OF_MRNA_ | | | | | |
| BINDING | | | | | |
| GO_REGULATION_ | 2/36 | 12/18046 | 0.000252186 | 0.005483238 | 3646/8663 |
| OF_RNA_BINDING | | | | | |
| GO_RIBOSOME_BIOGENESIS | 5/36 | 290/18046 | 0.00025949 | 0.005485334 | 55127/ |
| | | | | | 4931/9816/ |
| | | | | | 5822/57647 |
| GO_MUSCLE_SYSTEM_ | 6/36 | 470/18046 | 0.000303193 | 0.006235933 | 8106/7168/ |
| PROCESS | | | | | 140465/7111/ |
| | | | | | 7171/79784 |
| GO_RIBOSOMAL_SMALL_ | 3/36 | 68/18046 | 0.000334246 | 0.00669371 | 55127/5822/ |
| SUBUNIT_BIOGENESIS | | | | | 57647 |
| GO_ACTIN_FILAMENT_ | 3/36 | 74/18046 | 0.000428805 | 0.008367202 | 7168/7171/ |
| BUNDLE | | | | | 79784 |
| GO_POSITIVE_REGULATION | 4/36 | 182/18046 | 0.000458168 | 0.008716652 | 5663/3646/ |
| OF_BINDING | | | | | 8663/4931 |
| GO_INCLUSION_BODY | 3/36 | 78/18046 | 0.000500491 | 0.009289594 | 5663/8106/ |
| | | | | | 9816 |
| GO_REGULATION_OF_ | 3/36 | 79/18046 | 0.000519536 | 0.009413494 | 8667/3646/ |
| TRANSLATIONAL_ | | | | | 27335 |
| INITIATION | | | | | |
| GO_ACTOMYOSIN_ | 4/36 | 194/18046 | 0.000582726 | 0.010078513 | 7168/7111/ |
| STRUCTURE_ | | | | | 79784/ |
| ORGANIZATION | | | | | 399687 |
| GO_VIRAL_GENE_ | 4/36 | 194/18046 | 0.000582726 | 0.010078513 | 8665/8666/ |
| EXPRESSION | | | | | 8661/51386 |
| GO_MYOSIN_II_COMPLEX | 2/36 | 20/18046 | 0.000718737 | 0.012154636 | 140465/79784 |
| GO_REGULATION_ | 5/36 | 381/18046 | 0.000898813 | 0.014869496 | 5663/5195/ |
| OF_BINDING | | | | | 3646/8663/ |
| | | | | | 4931 |
| GO_RRNA_METABOLIC_ | 4/36 | 221/18046 | 0.000948179 | 0.015352431 | 55127/4931/ |
| PROCESS | | | | | 5822/57647 |
| GO_90S_PRERIBOSOME | 2/36 | 32/18046 | 0.001848268 | 0.029302757 | 55127/5822 |
| GO_SMOOTH_ | 2/36 | 34/18046 | 0.002085251 | 0.032385224 | 5663/4644 |
| ENDOPLASMIC_ | | | | | |
| RETICULUM | | | | | |
| GO_FIBRILLAR_CENTER | 3/36 | 130/18046 | 0.002192275 | 0.032712189 | 55127/5195/ |
| | | | | | 51386 |
| GO_RIBONUCLEOPROTEIN_ | 3/36 | 130/18046 | 0.002192275 | 0.032712189 | 10985/ |
| COMPLEX_BINDING | | | | | 27335/4931 |
| GO_REGULATION_OF_ | 5/36 | 484/18046 | 0.002579566 | 0.036640943 | 10985/8667/ |
| CELLULAR_ | | | | | 3646/8663/ |
| AMIDE_METABOLIC_ | | | | | 27335 |
| PROCESS | | | | | |
| GO_AGGRESOME | 2/36 | 38/18046 | 0.002600014 | 0.036640943 | 5663/9816 |
| GO_SMALL_SUBUNIT_ | 2/36 | 38/18046 | 0.002600014 | 0.036640943 | 55127/5822 |
| PROCESSOME | | | | | |
| GO_ADP_BINDING | 2/36 | 39/18046 | 0.002737128 | 0.037871892 | 399687/4646 |
| GO_AZUROPHIL_GRANULE | 3/36 | 155/18046 | 0.00360493 | 0.04898842 | 5663/10043/ |
| | | | | | 54472 |

TABLE 9B
CLUSTER 2

| Description | GeneRatio | BgRatio | pvalue | p.adjust | geneID |
|---|---|---|---|---|---|
| GO_RIBOSOME_BIOGENESIS | 21/110 | 290/18046 | 5.36E−17 | 9.42E−14 | 9136/6838/10199/ |
| | | | | | 9875/10775/23517/ |
| | | | | | 10153/10607/ |
| | | | | | 1662/9790/55035/ |
| | | | | | 25983/134430/ |
| | | | | | 11340/10200/ |
| | | | | | 79954/55759/ |
| | | | | | 65083/56915/ |
| | | | | | 51010/26574 |
| GO_RRNA_METABOLIC_ | 18/110 | 221/18046 | 1.41E−15 | 1.24E−12 | 9136/10199/9875/ |
| PROCESS | | | | | 10775/23517/ |
| | | | | | 10607/1662/9790/ |
| | | | | | 55035/25983/ |
| | | | | | 134430/11340/ |
| | | | | | 10200/79954/ |
| | | | | | 55759/65083/56915/ |
| | | | | | 51010 |
| GO_RIBONUCLEOPROTEIN_ | 22/110 | 419/18046 | 7.55E−15 | 4.43E−12 | 25980/9136/6838/ |
| COMPLEX_BIOGENESIS | | | | | 10199/9875/ |
| | | | | | 10775/23517/10153/ |
| | | | | | 10607/1662/ |
| | | | | | 9790/55035/25983/ |
| | | | | | 134430/11340/ |
| | | | | | 10200/79954/ |
| | | | | | 55759/65083/ |
| | | | | | 56915/51010/ |
| | | | | | 26574 |
| GO_NCRNA_PROCESSING | 18/110 | 378/18046 | 1.39E−11 | 6.10E−09 | 9136/10199/9875/ |
| | | | | | 10775/23517/ |
| | | | | | 10607/1662/9790/ |
| | | | | | 55035/25983/ |
| | | | | | 134430/11340/ |
| | | | | | 10200/79954/55759/ |
| | | | | | 65083/56915/ |
| | | | | | 51010 |
| GO_NCRNA_METABOLIC_ | 19/110 | 471/18046 | 6.21E−11 | 2.18E−08 | 9136/10199/9875/ |
| PROCESS | | | | | 10775/23517/ |
| | | | | | 10607/1662/9790/ |
| | | | | | 55035/56257/ |
| | | | | | 25983/134430/ |
| | | | | | 11340/10200/79954/ |
| | | | | | 55759/65083/ |
| | | | | | 56915/51010 |
| GO_CILIARY_BASAL_ | 10/110 | 95/18046 | 3.06E−10 | 8.98E−08 | 5116/5566/5577/ |
| BODY_PLASMA_ | | | | | 5108/9662/55755/ |
| MEMBRANE_DOCKING | | | | | 10142/11190/ |
| | | | | | 22994/22981 |
| GO_PRERIBOSOME | 9/110 | 77/18046 | 9.52E−10 | 2.39E−07 | 9136/10199/10607/ |
| | | | | | 9790/25983/ |
| | | | | | 134430/79954/ |
| | | | | | 55759/65083 |
| GO_SMALL_SUBUNIT_ | 7/110 | 38/18046 | 2.78E−09 | 6.12E−07 | 9136/10199/10607/ |
| PROCESSOME | | | | | 25983/134430/ |
| | | | | | 79954/65083 |
| GO_REGULATION_OF_MRNA_ | 12/110 | 199/18046 | 3.17E−09 | 6.20E−07 | 79675/26986/8531/ |
| CATABOLIC_PROCESS | | | | | 8761/23367/ |
| | | | | | 4343/26058/8087/ |
| | | | | | 9513/11340/ |
| | | | | | 56915/51010 |
| GO_RIBONUCLEOPROTEIN_ | 10/110 | 130/18046 | 6.76E−09 | 1.19E−06 | 26046/8531/1460/ |
| COMPLEX_BINDING | | | | | 23367/90850/ |
| | | | | | 25875/6731/6728/ |
| | | | | | 6729/55759 |
| GO_MATURATION_OF_ | 6/110 | 26/18046 | 9.32E−09 | 1.49E−06 | 9875/23517/11340/ |
| 5_8S_RRNA | | | | | 10200/55759/ |
| | | | | | 51010 |
| GO_NUCLEAR_EXOSOME_ | 5/110 | 16/18046 | 3.18E−08 | 4.66E−06 | 23517/11340/ |
| RNASE_COMPLEX | | | | | 10200/56915/ |
| | | | | | 51010 |
| GO_90S_PRERIBOSOME | 6/110 | 32/18046 | 3.56E−08 | 4.82E−06 | 10199/10607/ |
| | | | | | 9790/134430/ |
| | | | | | 55759/65083 |
| GO_MEMBRANE_DOCKING | 10/110 | 179/18046 | 1.43E−07 | 1.79E−05 | 5116/5566/5577/ |
| | | | | | 5108/9662/55755/ |
| | | | | | 10142/11190/ |
| | | | | | 22994/22981 |
| GO_EXORIBONUCLEASE_ | 5/110 | 26/18046 | 4.56E−07 | 5.01E−05 | 23517/11340/ |
| COMPLEX | | | | | 10200/56915/ |
| | | | | | 51010 |
| GO_MICROTUBULE_ | 5/110 | 26/18046 | 4.56E−07 | 5.01E−05 | 10426/10844/2801/ |
| NUCLEATION | | | | | 51199/10142 |
| GO_REGULATION_OF_MRNA_ | 12/110 | 325/18046 | 6.90E−07 | 7.14E−05 | 79675/26986/8531/ |
| METABOLIC_PROCESS | | | | | 8761/23367/ |
| | | | | | 4343/26058/808 |
| | | | | | 7/9513/11340/ |
| | | | | | 56915/51010 |
| GO_REGULATION_OF_CELL_ | 10/110 | 214/18046 | 7.45E−07 | 7.28E−05 | 5116/5566/5577/ |
| CYCLE_G2_M_PHASE_ | | | | | 5108/9662/55755/ |
| TRANSITION | | | | | 10142/11190/ |
| | | | | | 22994/22981 |
| GO_RNA_CATABOLIC_ | 13/110 | 404/18046 | 1.09E−06 | 0.000100685 | 79675/26986/8531/ |
| PROCESS | | | | | 8761/23367/ |
| | | | | | 4343/26058/23517/ |
| | | | | | 8087/9513/11340/ |
| | | | | | 56915/51010 |
| GO_MRNA_3_UTR_BINDING | 7/110 | 90/18046 | 1.27E−06 | 0.000111771 | 26986/8531/8761/ |
| | | | | | 23367/8087/ |
| | | | | | 9513/11340 |
| GO_CELL_CYCLE_G2_M_ | 10/110 | 271/18046 | 6.19E−06 | 0.000518826 | 5116/5566/5577/ |
| PHASE_TRANSITION | | | | | 5108/9662/55755/ |
| | | | | | 10142/11190/ |
| | | | | | 22994/22981 |
| GO_MICROTUBULE_ | 6/110 | 76/18046 | 6.91E−06 | 0.000543595 | 10426/10844/ |
| POLYMERIZATION | | | | | 2801/51199/55755/ |
| | | | | | 10142 |
| GO_MATURATION_OF_ | 4/110 | 21/18046 | 7.22E−06 | 0.000543595 | 9875/11340/ |
| 5_8S_RRNA_FROM_ | | | | | 55759/51010 |
| TRICISTRONIC_RRNA_ | | | | | |
| TRANSCRIPT_SSU_RRNA_ | | | | | |
| 5_8S_RRNA_LSU_RRNA | | | | | |
| GO_REGULATION_OF_CELL_ | 13/110 | 482/18046 | 7.51E−06 | 0.000543595 | 5116/5566/5577/ |
| CYCLE_PHASE_TRANSITION | | | | | 5108/9662/55755/ |
| | | | | | 10142/11190/ |
| | | | | | 22994/22981/ |
| | | | | | 26058/1642/ |
| | | | | | 56257 |
| GO_SNRNA_METABOLIC_ | 5/110 | 45/18046 | 7.73E−06 | 0.000543595 | 23517/56257/ |
| PROCESS | | | | | 11340/56915/ |
| | | | | | 51010 |
| GO_MICROTUBULE_ | 7/110 | 133/18046 | 1.71E−05 | 0.001155066 | 2801/5108/ |
| ORGANIZING_ | | | | | 9662/ |
| CENTER_ORGANIZATION | | | | | 51199/55755/ |
| | | | | | 11190/22994 |
| GO_CAMP_DEPENDENT_ | 3/110 | 10/18046 | 2.56E−05 | 0.001554821 | 5576/5566/5577 |
| PROTEIN_ | | | | | |
| KINASE_COMPLEX | | | | | |
| GO_MICROTUBULE_ | 3/110 | 10/18046 | 2.56E−05 | 0.001554821 | 5108/51199/ |
| ANCHORING_AT_ | | | | | 22981 |
| CENTROSOME | | | | | |
| GO_NUCLEAR_ | 3/110 | 10/18046 | 2.56E−05 | 0.001554821 | 11340/56915/ |
| TRANSCRIBED_ | | | | | 51010 |
| MRNA_CATABOLIC_ | | | | | |
| PROCESS_ | | | | | |
| EXONUCLEOLYTIC_3_5 | | | | | |
| GO_MICROBODY_ | 5/110 | 60/18046 | 3.21E−05 | 0.001883182 | 3615/11001/ |
| MEMBRANE | | | | | 8540/ |
| | | | | | 2181/84896 |
| GO_CYTOPLASMIC_STRESS_ | 5/110 | 63/18046 | 4.07E−05 | 0.002264705 | 26986/8761/23367/ |
| GRANULE | | | | | 4343/26058 |
| GO_PROTEIN_LOCALIZATION | 4/110 | 32/18046 | 4.12E−05 | 0.002264705 | 2804/5108/11190/ |
| TO_MICROTUBULE_ | | | | | 22994 |
| ORGANIZING_CENTER | | | | | |
| GO_MICROTUBULE_ | 3/110 | 12/18046 | 4.66E−05 | 0.002482798 | 5108/51199/ |
| ANCHORING_AT_ | | | | | 22981 |
| MICROTUBULE_ | | | | | |
| ORGANIZING_ | | | | | |
| CENTER | | | | | |
| GO_MICROTUBULE | 11/110 | 421/18046 | 5.25E−05 | 0.00264541 | 10426/10844/ |
| | | | | | 6902/10513/5116/ |
| | | | | | 2801/51199/ |
| | | | | | 55755/51361/ |
| | | | | | 22981/55829 |
| GO_NCRNA_CATABOLIC_ | 4/110 | 34/18046 | 5.26E−05 | 0.00264541 | 23517/11340/ |
| PROCESS | | | | | 56915/51010 |
| GO_CIS_GOLGI_NETWORK | 5/110 | 68/18046 | 5.90E−05 | 0.002718958 | 286451/2801/ |
| | | | | | 2804/10142/26229 |
| GO_RIBOSOMAL_SMALL_ | 5/110 | 68/18046 | 5.90E−05 | 0.002718958 | 6838/10607/9790/ |
| SUBUNIT_BIOGENESIS | | | | | 25983/79954 |
| GO_MATURATION_OF_SSU_ | 4/110 | 35/18046 | 5.92E−05 | 0.002718958 | 10607/9790/ |
| RRNA_FROM_ | | | | | 25983/79954 |
| TRICISTRONIC_RRNA_ | | | | | |
| TRANSCRIPT_SSU_RRNA_ | | | | | |
| 5_8S_RRNA_LSU_RRNA | | | | | |
| GO_CENTRIOLE_CENTRIOLE_ | 3/110 | 13/18046 | 6.03E−05 | 0.002718958 | 9662/51199/ |
| COHESION | | | | | 11190 |
| GO_MICROTUBULE_ | 6/110 | 114/18046 | 6.99E−05 | 0.003074733 | 10426/10844/ |
| POLYMERIZATION_ | | | | | 2801/51199/ |
| OR_DEPOLYMERIZATION | | | | | 55755/10142 |
| GO_CYTOPLASMIC_ | 3/110 | 14/18046 | 7.64E−05 | 0.003277085 | 11340/ |
| EXOSOME_RNASE_ | | | | | 56915/ |
| COMPLEX | | | | | 51010 |
| GO_ | 4/110 | 38/18046 | 8.22E−05 | 0.003443648 | 1459/1460/ |
| PHOSPHATIDYLCHOLINE_ | | | | | 1457/ |
| BIOSYNTHETIC_PROCESS | | | | | 2181 |
| GO_RNA_SURVEILLANCE | 3/110 | 15/18046 | 9.51E−05 | 0.003888504 | 11340/56915/ |
| | | | | | 51010 |
| GO_CILIUM_ORGANIZATION | 10/110 | 381/18046 | 0.000112518 | 0.00449817 | 5116/5566/5577/ |
| | | | | | 5108/9662/55755/ |
| | | | | | 10142/11190/ |
| | | | | | 22994/22981 |
| GO_ACTIVATION_ | 3/110 | 18/18046 | 0.000168219 | 0.006575502 | 5576/5566/5577 |
| OF_PROTEIN_ | | | | | |
| KINASE_A_ACTIVITY | | | | | |
| GO_REGULATION_ | 11/110 | 484/18046 | 0.000180131 | 0.006888039 | 26046/26986/ |
| OF_CELLULAR_ | | | | | 8531/23367/ |
| AMIDE_METABOLIC_ | | | | | 4343/9470/ |
| PROCESS | | | | | 26058/90850/ |
| | | | | | 8087/9513/25983 |
| GO_MATURATION_OF_ | 4/110 | 47/18046 | 0.000190485 | 0.007129015 | 10607/9790/ |
| SSU_RRNA | | | | | 25983/79954 |
| GO_RRNA_CATABOLIC_ | 3/110 | 19/18046 | 0.000198875 | 0.007287949 | 11340/56915/ |
| PROCESS | | | | | 51010 |
| GO_PROTEIN_KINASE_ | 4/110 | 49/18046 | 0.000224166 | 0.007914213 | 5576/5566/5577/ |
| A_BINDING | | | | | 10142 |
| GO_CENTRIOLE | 6/110 | 141/18046 | 0.000224963 | 0.007914213 | 10426/5116/5108/ |
| | | | | | 9662/51199/ |
| | | | | | 11190 |
| GO_GOLGI_ORGANIZATION | 6/110 | 142/18046 | 0.000233737 | 0.008061652 | 2801/2804/9659/ |
| | | | | | 10142/64689/ |
| | | | | | 51361 |
| GO_GAMMA_TUBULIN_ | 3/110 | 21/18046 | 0.000270553 | 0.008979313 | 10426/10844/ |
| COMPLEX | | | | | 55755 |
| GO_PERICENTRIOLAR_ | 3/110 | 21/18046 | 0.000270553 | 0.008979313 | 5108/51199/ |
| MATERIAL | | | | | 55755 |
| GO_CYTOPLASMIC_ | 4/110 | 53/18046 | 0.000304068 | 0.009904742 | 10426/ |
| MICROTUBULE_ | | | | | 10844/5108/ |
| ORGANIZATION | | | | | 51361 |
| GO_POSITIVE_REGULATION_ | 6/110 | 153/18046 | 0.000349203 | 0.011168142 | 1459/5116/5566/ |
| OF_INTRACELLULAR_ | | | | | 5108/22994/ |
| PROTEIN_TRANSPORT | | | | | 26229 |
| GO_RIBOSOME_BINDING | 4/110 | 57/18046 | 0.00040258 | 0.012645311 | 90850/25875/ |
| | | | | | 6731/6728 |
| GO_PROTEIN_ | 4/110 | 58/18046 | 0.000430385 | 0.013281524 | 2804/5108/ |
| LOCALIZATION_ | | | | | 11190/ |
| TO_CYTOSKELETON | | | | | 22994 |
| GO_REGULATION_OF_ | 7/110 | 227/18046 | 0.000482586 | 0.014635663 | 1459/5116/5566/ |
| INTRACELLULAR_ | | | | | 5108/56850/ |
| PROTEIN_TRANSPORT | | | | | 22994/26229 |
| GO_RIBONUCLEOPROTEIN_ | 7/110 | 229/18046 | 0.000508497 | 0.01467639 | 26986/8761/ |
| GRANULE | | | | | 23367/4343/ |
| | | | | | 26058/ |
| | | | | | 8087/9513 |
| GO_CELLULAR_ | 3/110 | 26/18046 | 0.000517303 | 0.01467639 | 5576/5566/5577 |
| RESPONSE_TO_ | | | | | |
| GLUCAGON_STIMULUS | | | | | |
| GO_GAMMA_TUBULIN_ | 3/110 | 26/18046 | 0.000517303 | 0.01467639 | 10426/10844/ |
| BINDING | | | | | 55755 |
| GO_MICROTUBULE_ | 3/110 | 26/18046 | 0.000517303 | 0.01467639 | 5108/51199/ |
| ANCHORING | | | | | 22981 |
| GO_SMALL_NUCLEOLAR_ | 3/110 | 27/18046 | 0.000579393 | 0.016177018 | 9136/10199/ |
| RIBONUCLEOPROTEIN_ | | | | | 10775 |
| COMPLEX | | | | | |
| GO_ACID_THIOL_LIGASE_ | 3/110 | 30/18046 | 0.000793603 | 0.021476108 | 8803/11001/ |
| ACTIVITY | | | | | 2181 |
| GO_SNRNA_3_END_ | 3/110 | 30/18046 | 0.000793603 | 0.021476108 | 11340/56915/ |
| PROCESSING | | | | | 51010 |
| GO_POSITIVE_REGULATION_ | 8/110 | 326/18046 | 0.000864162 | 0.022872592 | 1459/5116/5566/ |
| OF_CELLULAR_PROTEIN_ | | | | | 5108/11190/22994/ |
| LOCALIZATION | | | | | 26229/2181 |
| GO_RENAL_SYSTEM_ | 5/110 | 121/18046 | 0.000871213 | 0.022872592 | 5576/5566/5577/ |
| PROCESS | | | | | 4643/1312 |
| GO_POSITIVE_REGULATION_ | 3/110 | 33/18046 | 0.001052412 | 0.02722342 | 51199/55755/ |
| OF_MICROTUBULE_ | | | | | 10142 |
| POLYMERIZATION_ | | | | | |
| OR_DEPOLYMERIZATION | | | | | |
| GO_POSITIVE_REGULATION_ | 5/110 | 129/18046 | 0.001160757 | 0.029590902 | 26986/8531/ |
| OF_TRANSLATION | | | | | 23367/8087/9513 |
| GO_CILIARY_BASE | 3/110 | 35/18046 | 0.001251353 | 0.030273116 | 5576/5566/5577 |
| GO_NUCLEAR_ | 3/110 | 35/18046 | 0.001251353 | 0.030273116 | 11340/56915/ |
| TRANSCRIBED_MRNA_ | | | | | 51010 |
| CATABOLIC_PROCESS_ | | | | | |
| EXONUCLEOLYTIC | | | | | |
| GO_POSITIVE_REGULATION_ | 3/110 | 35/18046 | 0.001251353 | 0.030273116 | 26986/23367/ |
| OF_VIRAL_GENOME_ | | | | | 1642 |
| REPLICATION | | | | | |
| GO_NUCLEAR_ | 4/110 | 77/18046 | 0.00125636 | 0.030273116 | 26986/11340/ |
| TRANSCRIBED_MRNA_ | | | | | 56915/51010 |
| CATABOLIC_PROCESS_ | | | | | |
| DEADENYLATION_ | | | | | |
| DEPENDENT_DECAY | | | | | |
| GO_RENAL_WATER_ | 3/110 | 36/18046 | 0.001359091 | 0.031875216 | 5576/5566/5577 |
| HOMEOSTASIS | | | | | |
| GO_SNRNA_PROCESSING | 3/110 | 36/18046 | 0.001359091 | 0.031875216 | 11340/56915/ |
| | | | | | 51010 |
| GO_MICROBODY | 5/110 | 135/18046 | 0.001420656 | 0.032880711 | 3615/11001/8540/ |
| | | | | | 2181/84896 |
| GO_RESPONSE_TO_ | 3/110 | 37/18046 | 0.001472489 | 0.033637768 | 5576/5566/5577 |
| GLUCAGON | | | | | |
| GO_CALCIUM_ | 2/110 | 10/18046 | 0.001604815 | 0.03573253 | 490/27032 |
| TRANSMEMBRANE_ | | | | | |
| TRANSPORTER_ACTIVITY_ | | | | | |
| PHOSPHORYLATIVE_ | | | | | |
| MECHANISM | | | | | |
| GO_CAMP_DEPENDENT_ | 2/110 | 10/18046 | 0.001604815 | 0.03573253 | 5576/5577 |
| PROTEIN_KINASE_ | | | | | |
| REGULATOR_ACTIVITY | | | | | |
| GO_AMMONIUM_ION_ | 6/110 | 206/18046 | 0.001646653 | 0.035763652 | 1459/1460/1457/ |
| METABOLIC_PROCESS | | | | | 5447/2181/1312 |
| GO_ | 4/110 | 83/18046 | 0.001659036 | 0.035763652 | 1459/1460/1457/ |
| PHOSPHATIDYLCHOLINE_ | | | | | 2181 |
| METABOLIC_PROCESS | | | | | |
| GO_TRANSLATION_ | 5/110 | 140/18046 | 0.001668098 | 0.035763652 | 26986/23367/ |
| REGULATOR_ACTIVITY | | | | | 9470/8087/9513 |
| GO_POSITIVE_REGULATION_ | 6/110 | 207/18046 | 0.00168754 | 0.035763652 | 1459/5116/5566/ |
| OF_INTRACELLULAR_ | | | | | 5108/22994/ |
| TRANSPORT | | | | | 26229 |
| GO_LIGASE_ACTIVITY_ | 3/110 | 40/18046 | 0.001847707 | 0.038691864 | 8803/11001/2181 |
| FORMING_CARBON_ | | | | | |
| SULFUR_BONDS | | | | | |
| GO_LEUCINE_ZIPPER_ | 2/110 | 11/18046 | 0.001953643 | 0.039499517 | 23085/26574 |
| DOMAIN_BINDING | | | | | |
| GO_MEDIUM_CHAIN_FATTY_ | 2/110 | 11/18046 | 0.001953643 | 0.039499517 | 11001/2181 |
| ACID_COA_LIGASE_ | | | | | |
| ACTIVITY | | | | | |
| GO_NEGATIVE_ | 2/110 | 11/18046 | 0.001953643 | 0.039499517 | 5576/5577 |
| REGULATION_ | | | | | |
| OF_CAMP_DEPENDENT_ | | | | | |
| PROTEIN_KINASE_ACTIVITY | | | | | |
| GO_RNA_PHOSPHODIESTER_ | 5/110 | 148/18046 | 0.002127914 | 0.042534098 | 4343/10775/11340/ |
| BOND_HYDROLYSIS | | | | | 56915/51010 |
| GO_GOLGI_STACK | 5/110 | 150/18046 | 0.002256012 | 0.043235401 | 286451/2802/2801/ |
| | | | | | 2804/10142 |
| GO_RNA_PHOSPHODIESTER_ | 3/110 | 43/18046 | 0.002277594 | 0.043235401 | 11340/56915/ |
| BOND_HYDROLYSIS_ | | | | | 51010 |
| EXONUCLEOLYTIC | | | | | |
| GO_PROTEIN_FOLDING | 6/110 | 220/18046 | 0.002292386 | 0.043235401 | 1459/1460/1457/ |
| | | | | | 6902/7841/ |
| | | | | | 131118 |
| GO_MICROTUBULE_MINUS_ | 2/110 | 12/18046 | 0.002335056 | 0.043235401 | 10426/10844 |
| END_BINDING | | | | | |
| GO_POSITIVE_REGULATION_ | 2/110 | 12/18046 | 0.002335056 | 0.043235401 | 2801/64689 |
| OF_UBIQUITIN_PROTEIN_ | | | | | |
| LIGASE_ACTIVITY | | | | | |
| GO_RNA_7_ | 2/110 | 12/18046 | 0.002335056 | 0.043235401 | 23367/9470 |
| METHYLGUANOSINE_ | | | | | |
| CAP_BINDING | | | | | |
| GO_SNORNA_3_END_ | 2/110 | 12/18046 | 0.002335056 | 0.043235401 | 56915/51010 |
| PROCESSING | | | | | |
| GO_POSITIVE_REGULATION_ | 5/110 | 156/18046 | 0.002674059 | 0.048150038 | 26986/8531/ |
| OF_CELLULAR_AMIDE_ | | | | | 23367/8087/9513 |
| METABOLIC_PROCESS | | | | | |
| GO_NEGATIVE_REGULATION_ | 6/110 | 228/18046 | 0.002738195 | 0.048150038 | 23367/4343/ |
| OF_CELLULAR_AMIDE_ | | | | | 9470/26058/ |
| METABOLIC_PROCESS | | | | | 8087/9513 |
| GO_LONG_CHAIN_FATTY_ | 2/110 | 13/18046 | 0.002748651 | 0.048150038 | 11001/2181 |
| ACID_COA_ | | | | | |
| LIGASE_ACTIVITY | | | | | |
| GO_PROTEIN_KINASE_A_ | 2/110 | 13/18046 | 0.002748651 | 0.048150038 | 5576/5577 |
| CATALYTIC_ | | | | | |
| SUBUNIT_BINDING | | | | | |
| GO_TRANSLATION_ | 2/110 | 13/18046 | 0.002748651 | 0.048150038 | 26986/23367 |
| ACTIVATOR_ACTIVITY | | | | | |
| GO_REGULATION_OF_ | 3/110 | 46/18046 | 0.002764726 | 0.048150038 | 5898/8087/9513 |
| FILOPODIUM_ASSEMBLY | | | | | |

TABLE 9C
CLUSTER 3

| Description | GeneRatio | BgRatio | pvalue | p.adjust | geneID |
|---|---|---|---|---|---|
| GO_UBIQUITIN_LIGASE_ | 7/54 | 284/18046 | 2.09E−05 | 0.021996873 | 51646/57610/ |
| COMPLEX | | | | | 10296/10048/ |
| | | | | | 80232/64795/54994 |

TABLE 9D
CLUSTER 4

| Description | GeneRatio | BgRatio | pvalue | p.adjust | geneID |
|---|---|---|---|---|---|
| GO_TELOMERE_ | 6/120 | 27/18046 | 2.01E−08 | 2.43E−05 | 5976/5422/ |
| MAINTENANCE_ | | | | | 5557/5558/ |
| VIA_SEMI_CONSERVATIVE_ | | | | | 23649/1763 |
| REPLICATION | | | | | |
| GO_GDP_BINDING | 8/120 | 74/18046 | 3.16E−08 | 2.43E−05 | 5878/7879/4218/ |
| | | | | | 5862/10890/51552/ |
| | | | | | 387/22931 |
| GO_RAB_PROTEIN_SIGNAL_ | 8/120 | 75/18046 | 3.51E−08 | 2.43E−05 | 5878/7879/4218/5862/ |
| TRANSDUCTION | | | | | 10890/51552/5861/ |
| | | | | | 22931 |
| GO_GOLGI_VESICLE_ | 14/120 | 367/18046 | 1.54E−07 | 7.98E−05 | 10897/10945/ |
| TRANSPORT | | | | | 1781/90522/ |
| | | | | | 23041/26958/57222/ |
| | | | | | 28952/54520/4218/ |
| | | | | | 10890/51552/5861/ |
| | | | | | 10960 |
| GO_DNA_POLYMERASE_ | 5/120 | 22/18046 | 2.88E−07 | 0.000119247 | 5422/5557/5558/ |
| COMPLEX | | | | | 23649/1763 |
| GO_RAS_PROTEIN_SIGNAL_ | 14/120 | 447/18046 | 1.63E−06 | 0.000564801 | 10146/9908/5962/ |
| TRANSDUCTION | | | | | 382/117178/ |
| | | | | | 5878/7879/4218/ |
| | | | | | 5862/10890/51552/ |
| | | | | | 387/5861/22931 |
| GO_COATED_VESICLE | 11/120 | 290/18046 | 3.76E−06 | 0.001081198 | 8546/10897/10945/ |
| | | | | | 90522/26958/ |
| | | | | | 161/57222/1173/ |
| | | | | | 4218/51552/10960 |
| GO_CELL_CYCLE_DNA_ | 6/120 | 64/18046 | 4.17E−06 | 0.001081198 | 5976/5422/5557/ |
| REPLICATION | | | | | 5558/23649/1763 |
| GO_CELLULAR_TRANSITION_ | 7/120 | 110/18046 | 8.71E−06 | 0.002006511 | 22/523/25800/23516/ |
| METAL_ION_HOMEOSTASIS | | | | | 10463/28982/28952 |
| GO_GTPASE_ACTIVITY | 11/120 | 323/18046 | 1.04E−05 | 0.002163487 | 382/5878/7879/4218/ |
| | | | | | 5862/10890/51552/ |
| | | | | | 387/5861/2787/22931 |
| GO_ENDOSOMAL_ | 9/120 | 228/18046 | 2.17E−05 | 0.00400846 | 8546/382/28952/ |
| TRANSPORT | | | | | 54520/23085/7879/ |
| | | | | | 4218/10890/51552 |
| GO_ENDOPLASMIC_ | 7/120 | 129/18046 | 2.47E−05 | 0.00400846 | 10897/10945/ |
| RETICULUM_GOLGI_ | | | | | 90522/26958/ |
| INTERMEDIATE_ | | | | | 57222/5862/10960 |
| COMPARTMENT | | | | | |
| GO_GOLGI_ASSOCIATED_ | 8/120 | 178/18046 | 2.51E−05 | 0.00400846 | 10897/10945/90522/ |
| VESICLE | | | | | 26958/57222/ |
| | | | | | 4218/51552/10960 |
| GO_REPLISOME | 4/120 | 27/18046 | 2.90E−05 | 0.004150096 | 5422/5557/5558/ |
| | | | | | 23649 |
| GO_TRANSITION_METAL_ | 7/120 | 133/18046 | 3.00E−05 | 0.004150096 | 22/523/25800/23516/ |
| ION_HOMEOSTASIS | | | | | 10463/28982/28952 |
| GO_ENDOPLASMIC_ | 8/120 | 207/18046 | 7.33E−05 | 0.009498922 | 10897/10945/ |
| RETICULUM_TO_ | | | | | 1781/90522/26958/ |
| GOLGI_VESICLE_ | | | | | 57222/5861/ |
| MEDIATED_TRANSPORT | | | | | 10960 |
| GO_ENDOCYTIC_VESICLE_ | 7/120 | 160/18046 | 9.71E−05 | 0.011308379 | 79971/161/1173/ |
| MEMBRANE | | | | | 7879/4218/10890/949 |
| GO_VACUOLAR_MEMBRANE | 11/120 | 414/18046 | 0.000100218 | 0.011308379 | 8546/10548/ |
| | | | | | 2040/523/161/ |
| | | | | | 1173/5878/7879/ |
| | | | | | 5862/51552/949 |
| GO_DNA_REPLICATION_ | 4/120 | 37/18046 | 0.000103646 | 0.011308379 | 5422/5557/5558/ |
| INITIATION | | | | | 23649 |
| GO_ANTIGEN_PROCESSING_ | 8/120 | 227/18046 | 0.000139042 | 0.014411745 | 8546/5714/1781/161/ |
| AND_PRESENTATION | | | | | 1173/3416/ |
| | | | | | 7879/10890 |
| GO_ENDOPLASMIC_ | 3/120 | 16/18046 | 0.000150747 | 0.014788353 | 57142/10890/22931 |
| RETICULUM_ | | | | | |
| TUBULAR_NETWORK_ | | | | | |
| ORGANIZATION | | | | | |
| GO_ENDOCYTIC_VESICLE | 9/120 | 296/18046 | 0.000162191 | 0.014788353 | 79971/161/1173/382/ |
| | | | | | 7879/4218/10890/ |
| | | | | | 51552/949 |
| GO_SECRETORY_GRANULE_ | 9/120 | 298/18046 | 0.000170572 | 0.014788353 | 2040/196527/161/ |
| MEMBRANE | | | | | 5878/7879/10890/ |
| | | | | | 51552/387/22931 |
| GO_NUCLEAR_ | 4/120 | 42/18046 | 0.000171211 | 0.014788353 | 5422/5557/5558/ |
| REPLICATION_FORK | | | | | 23649 |
| GO_MYOSIN_V_BINDING | 3/120 | 17/18046 | 0.000182163 | 0.014974885 | 4218/10890/51552 |
| GO_TRANSITION_METAL_ | 6/120 | 125/18046 | 0.000187818 | 0.014974885 | 22/523/25800/23516/ |
| ION_TRANSPORT | | | | | 10463/28982 |
| GO_LIPID_DROPLET | 5/120 | 82/18046 | 0.000216849 | 0.016615957 | 10280/1727/5878/ |
| | | | | | 7879/23111 |
| GO_ENDOCYTIC_RECYCLING | 4/120 | 45/18046 | 0.000224432 | 0.016615957 | 382/28952/54520/ |
| | | | | | 51552 |
| GO_RETROGRADE_VESICLE_ | 5/120 | 86/18046 | 0.000270997 | 0.019371637 | 10945/26958/57222/ |
| MEDIATED_TRANSPORT_ | | | | | 5861/10960 |
| GOLGI_TO_ | | | | | |
| ENDOPLASMIC_RETICULUM | | | | | |
| GO_GUANYL_NUCLEOTIDE_ | 10/120 | 396/18046 | 0.000314413 | 0.02172592 | 382/5878/7879/4218/ |
| BINDING | | | | | 5862/10890/51552/ |
| | | | | | 387/5861/22931 |
| GO_ENDOPLASMIC_ | 3/120 | 21/18046 | 0.00034944 | 0.023367362 | 57142/10890/22931 |
| RETICULUM_ | | | | | |
| TUBULAR_NETWORK | | | | | |
| GO_DNA_DEPENDENT_DNA_ | 6/120 | 146/18046 | 0.000433554 | 0.028086201 | 5976/5422/5557/ |
| REPLICATION | | | | | 5558/23649/1763 |
| GO_ENDOPLASMIC_ | 4/120 | 57/18046 | 0.000559585 | 0.034128969 | 57142/10890/ |
| RETICULUM_ | | | | | 10960/22931 |
| ORGANIZATION | | | | | |
| GO_POST_GOLGI_ | 5/120 | 101/18046 | 0.000569486 | 0.034128969 | 23041/28952/ |
| VESICLE_MEDIATED_ | | | | | 54520/10890/ |
| TRANSPORT | | | | | 51552 |
| GO_CLATHRIN_ADAPTOR_ | 3/120 | 25/18046 | 0.000592688 | 0.034128969 | 8546/161/1173 |
| COMPLEX | | | | | |
| GO_ENDOPLASMIC_ | 3/120 | 25/18046 | 0.000592688 | 0.034128969 | 57142/10890/22931 |
| RETICULUM_ | | | | | |
| SUBCOMPARTMENT | | | | | |
| GO_ENDOMEMBRANE_ | 10/120 | 436/18046 | 0.0006663 | 0.036683711 | 196527/57142/ |
| SYSTEM_ | | | | | 26993/7879/5862/ |
| ORGANIZATION | | | | | 10890/5861/ |
| | | | | | 10960/26092/22931 |
| GO_MAINTENANCE_OF_ | 5/120 | 105/18046 | 0.000679769 | 0.036683711 | 10945/9908/28952/ |
| PROTEIN_LOCATION | | | | | 2200/2201 |
| GO_ATPASE_ACTIVITY | 10/120 | 438/18046 | 0.000690142 | 0.036683711 | 22/481/1781/ |
| | | | | | 79572/10146/5976/ |
| | | | | | 1763/3416/ |
| | | | | | 10632/2963 |
| GO_PIGMENT_GRANULE | 5/120 | 106/18046 | 0.000709675 | 0.036778921 | 2040/5878/7879/ |
| | | | | | 5862/5861 |
| GO_ZINC_ION_TRANSPORT | 3/120 | 27/18046 | 0.000746478 | 0.037742667 | 25800/23516/10463 |
| GO_RNA_POLYMERASE_ | 5/120 | 112/18046 | 0.00091026 | 0.044927838 | 5422/5557/5558/ |
| COMPLEX | | | | | 23649/2963 |
| GO_RETROGRADE_ | 3/120 | 30/18046 | 0.001021201 | 0.049231367 | 28952/54520/4218 |
| TRANSPORT_ | | | | | |
| ENDOSOME_TO_PLASMA_ | | | | | |
| MEMBRANE | | | | | |

TABLE 9E
CLUSTER 5

| Description | GeneRatio | BgRatio | pvalue | p.adjust | geneID |
|---|---|---|---|---|---|
| GO_DNA_DEALKYLATION_ | 3/113 | 10/18046 | 2.78E−05 | 0.03091315 | 10973/51008/84164 |
| INVOLVED_IN_DNA_REPAIR | | | | | |
| GO_CHAPERONE_BINDING | 6/113 | 102/18046 | 4.36E−05 | 0.03091315 | 4189/7157/8975/3337/ |
| | | | | | 11080/26520 |
| GO_FATTY_ACID_ | 6/113 | 104/18046 | 4.86E−05 | 0.03091315 | 2475/33/10005/3295/ |
| CATABOLIC_PROCESS | | | | | 11001/10999 |
| GO_CELLULAR_LIPID_ | 8/113 | 212/18046 | 5.66E−05 | 0.03091315 | 2475/33/10005/ |
| CATABOLIC_PROCESS | | | | | 3295/11001/10999/ |
| | | | | | 26090/284161 |
| GO_COENZYME_BINDING | 9/113 | 287/18046 | 8.09E−05 | 0.03091315 | 9517/33/7296/ |
| | | | | | 55034/10243/1727/ |
| | | | | | 23530/64757/5033 |
| GO_FATTY_ACID_BETA_ | 5/113 | 71/18046 | 8.25E−05 | 0.03091315 | 2475/33/10005/3295/ |
| OXIDATION | | | | | 11001 |
| GO_ORGANELLE_ | 10/113 | 382/18046 | 0.000143908 | 0.043242626 | 79971/25923/79586/ |
| SUBCOMPARTMENT | | | | | 23256/2530/55717/ |
| | | | | | 55968/3482/2590/6786 |
| GO_MONOCARBOXYLIC_ | 6/113 | 128/18046 | 0.000153888 | 0.043242626 | 2475/33/10005/ |
| ACID_CATABOLIC_ | | | | | 3295/11001/10999 |
| PROCESS | | | | | |
| GO_MANNOSE_BINDING | 3/113 | 19/18046 | 0.000215323 | 0.049194879 | 81562/3482/3998 |
| GO_PROTEIN_ | 8/113 | 266/18046 | 0.000270323 | 0.049194879 | 23534/6774/ |
| LOCALIZATION_ | | | | | 7704/51366/7157/ |
| TO_NUCLEUS | | | | | 163590/10527/55027 |
| GO_NUCLEAR_ENVELOPE_ | 4/113 | 51/18046 | 0.000290316 | 0.049194879 | 79188/55968/5520/26993 |
| ORGANIZATION | | | | | |
| GO_OUTER_MEMBRANE | 7/113 | 204/18046 | 0.000298904 | 0.049194879 | 140707/54708/2475/1727/ |
| | | | | | 64757/51566/23098 |
| GO_CELL_CYCLE_G2_M_ | 8/113 | 271/18046 | 0.000306374 | 0.049194879 | 7157/4361/5520/9113/ |
| PHASE_TRANSITION | | | | | 5704/55722/26993/5715 |
| GO_ORGANIC_ACID_ | 8/113 | 271/18046 | 0.000306374 | 0.049194879 | 2475/33/10005/3295/ |
| CATABOLIC_PROCESS | | | | | 11001/10999/51449/501 |

TABLE 9F
CLUSTER 6

| Description | GeneRatio | BgRatio | pvalue | p.adjust | geneID |
|---|---|---|---|---|---|
| GO_STRUCTURAL_ | 6/74 | 28/18046 | 1.36E−09 | 1.49E−06 | 10204/8021/ |
| CONSTITUENT_OF_ | | | | | 23636/53371/ |
| NUCLEAR_PORE | | | | | 4927/9818 |
| GO_PROTEIN_TARGETING_ | 7/74 | 101/18046 | 1.85E−07 | 0.000101221 | 9512/23203/ |
| TO_MITOCHONDRION | | | | | 10531/26519/ |
| | | | | | 90580/26515/ |
| | | | | | 26520 |
| GO_NCRNA_EXPORT_FROM_ | 5/74 | 38/18046 | 4.57E−07 | 0.000166955 | 8021/23636/ |
| NUCLEUS | | | | | 53371/ |
| | | | | | 4927/9818 |
| GO_PROTEIN_ | 7/74 | 141/18046 | 1.78E−06 | 0.000457903 | 9512/23203/ |
| LOCALIZATION_ | | | | | 10531/26519/ |
| TO_MITOCHONDRION | | | | | 90580/26515/ |
| | | | | | 26520 |
| GO_NUCLEAR_PORE | 6/74 | 92/18046 | 2.09E−06 | 0.000457903 | 10204/8021/ |
| | | | | | 23636/53371/ |
| | | | | | 4927/9818 |
| GO_MULTI_ORGANISM_ | 5/74 | 62/18046 | 5.45E−06 | 0.000996971 | 8021/23636/ |
| LOCALIZATION | | | | | 53371/4927/ |
| | | | | | 9818 |
| GO_PROTEIN_TARGETING | 10/74 | 428/18046 | 9.39E−06 | 0.001347342 | 9512/23203/ |
| | | | | | 10531/5189/ |
| | | | | | 252983/26519/ |
| | | | | | 90580/26515/ |
| | | | | | 26520/53371 |
| GO_PROTEIN_INSERTION_ | 3/74 | 11/18046 | 1.07E−05 | 0.001347342 | 26519/90580/ |
| INTO_ | | | | | 26520 |
| MITOCHONDRIAL_INNER_ | | | | | |
| MEMBRANE | | | | | |
| GO_MITOCHONDRIAL_ | 8/74 | 260/18046 | 1.11E−05 | 0.001347342 | 9512/23203/ |
| PROTEIN_COMPLEX | | | | | 26519/90580/ |
| | | | | | 55735/26515/ |
| | | | | | 26520/51116 |
| GO_PROTEIN_IMPORT | 7/74 | 192/18046 | 1.36E−05 | 0.001391633 | 10204/5189/ |
| | | | | | 8021/23636/ |
| | | | | | 53371/4927/ |
| | | | | | 9818 |
| GO_HOST_ | 5/74 | 75/18046 | 1.40E−05 | 0.001391633 | 8021/23636/ |
| CELLULAR_COMPONENT | | | | | 53371/ |
| | | | | | 4927/9818 |
| GO_REGULATION_ | 5/74 | 79/18046 | 1.80E−05 | 0.001644711 | 8021/23636/ |
| OF_CELLULAR_ | | | | | 53371/ |
| RESPONSE_TO_HEAT | | | | | 4927/9818 |
| GO_PROTEIN_SUMOYLATION | 5/74 | 81/18046 | 2.03E−05 | 0.001715002 | 8021/23636/53371/ |
| | | | | | 4927/9818 |
| GO_REGULATION_OF_ | 5/74 | 87/18046 | 2.88E−05 | 0.002222589 | 8021/23636/ |
| CARBOHYDRATE_ | | | | | 53371/4927/ |
| CATABOLIC_PROCESS | | | | | 9818 |
| GO_ORGANELLE_ENVELOPE_ | 5/74 | 88/18046 | 3.04E−05 | 0.002222589 | 2671/26519/90580/ |
| LUMEN | | | | | 26515/26520 |
| GO_MRNA_TRANSPORT | 6/74 | 151/18046 | 3.60E−05 | 0.002469664 | 10204/8021/23636/ |
| | | | | | 53371/4927/9818 |
| GO_INNER_MITOCHONDRIAL_ | 4/74 | 47/18046 | 4.07E−05 | 0.002623514 | 26519/90580/ |
| MEMBRANE_ | | | | | 55735/26520 |
| ORGANIZATION | | | | | |
| GO_ESTABLISHMENT_ | 3/74 | 17/18046 | 4.32E−05 | 0.002632126 | 26519/90580/ |
| OF_PROTEIN_ | | | | | 26520 |
| LOCALIZATION_TO_ | | | | | |
| MITOCHONDRIAL_MEMBRANE | | | | | |
| GO_IMPORT_INTO_NUCLEUS | 6/74 | 164/18046 | 5.72E−05 | 0.0032999 | 10204/8021/ |
| | | | | | 23636/53371/ |
| | | | | | 4927/9818 |
| GO_REGULATION_OF_ | 5/74 | 105/18046 | 7.10E−05 | 0.003892571 | 10204/8021/ |
| NUCLEOCYTOPLASMIC_ | | | | | 23636/ |
| TRANSPORT | | | | | 53371/9818 |
| GO_MITOCHONDRIAL_ | 7/74 | 258/18046 | 8.96E−05 | 0.00445808 | 9512/23203/ |
| TRANSPORT | | | | | 10531/26519/ |
| | | | | | 90580/26515/ |
| | | | | | 26520 |
| GO_MRNA_EXPORT_FROM_ | 5/74 | 111/18046 | 9.24E−05 | 0.00445808 | 8021/23636/ |
| NUCLEUS | | | | | 53371/4927/9818 |
| GO_REGULATION_OF_ | 4/74 | 58/18046 | 9.35E−05 | 0.00445808 | 10204/23636/ |
| PROTEIN_IMPORT | | | | | 53371/9818 |
| GO_REGULATION_OF_ | 5/74 | 116/18046 | 0.000113831 | 0.005203046 | 8021/23636/ |
| POSTTRANSCRIPTIONAL_ | | | | | 53371/ |
| GENE_SILENCING | | | | | 4927/9818 |
| GO_REGULATION_ | 5/74 | 118/18046 | 0.000123389 | 0.005414302 | 8021/23636/ |
| OF_NUCLEOTIDE_ | | | | | 53371/ |
| METABOLIC_PROCESS | | | | | 4927/9818 |
| GO_REGULATION_OF_ATP_ | 5/74 | 121/18046 | 0.000138865 | 0.005577514 | 8021/23636/ |
| METABOLIC_PROCESS | | | | | 53371/4927/9818 |
| GO_VIRAL_GENE_ | 6/74 | 194/18046 | 0.000144237 | 0.005577514 | 22954/8021/ |
| EXPRESSION | | | | | 23636/53371/ |
| | | | | | 4927/9818 |
| GO_ADP_METABOLIC_ | 5/74 | 122/18046 | 0.00014434 | 0.005577514 | 8021/23636/53371/ |
| PROCESS | | | | | 4927/9818 |
| GO_NUCLEAR_EXPORT | 6/74 | 195/18046 | 0.000148338 | 0.005577514 | 10204/8021/23636/ |
| | | | | | 53371/4927/9818 |
| GO_ESTABLISHMENT_OF_ | 6/74 | 196/18046 | 0.00015253 | 0.005577514 | 10204/8021/ |
| RNA_LOCALIZATION | | | | | 23636/53371/ |
| | | | | | 4927/9818 |
| GO_NUCLEOTIDE_ | 5/74 | 134/18046 | 0.00022379 | 0.007919271 | 8021/23636/53371/ |
| PHOSPHORYLATION | | | | | 4927/9818 |
| GO_RNA_EXPORT_FROM_ | 5/74 | 136/18046 | 0.000239741 | 0.008218637 | 8021/23636/53371/ |
| NUCLEUS | | | | | 4927/9818 |
| GO_FLEMMING_BODY | 3/74 | 31/18046 | 0.000273955 | 0.00910694 | 11064/55165/ |
| | | | | | 23636 |
| GO_CENTRIOLE | 5/74 | 141/18046 | 0.00028341 | 0.009144139 | 8481/8924/55165/ |
| | | | | | 145508/49856 |
| GO_CELLULAR_RESPONSE_ | 5/74 | 142/18046 | 0.000292823 | 0.009177922 | 8021/23636/53371/ |
| TO_HEAT | | | | | 4927/9818 |
| GO_RNA_LOCALIZATION | 6/74 | 229/18046 | 0.000352829 | 0.010751487 | 10204/8021/23636/ |
| | | | | | 53371/4927/9818 |
| GO_PYRUVATE_METABOLIC_ | 5/74 | 150/18046 | 0.00037694 | 0.011175752 | 8021/23636/53371/ |
| PROCESS | | | | | 4927/9818 |
| GO_NUCLEOSIDE_ | 5/74 | 154/18046 | 0.000425286 | 0.011962536 | 8021/23636/ |
| DIPHOSPHATE_ | | | | | 53371/4927/ |
| METABOLIC_PROCESS | | | | | 9818 |
| GO_REGULATION_OF_GENE | 5/74 | 154/18046 | 0.000425286 | 0.011962536 | 8021/23636/53371/ |
| SILENCING | | | | | 4927/9818 |
| GO_NUCLEOBASE_ | 6/74 | 240/18046 | 0.000452662 | 0.012414247 | 10204/8021/ |
| CONTAINING_COMPOUND_ | | | | | 23636/53371/ |
| TRANSPORT | | | | | 4927/9818 |
| GO_REGULATION_ | 5/74 | 157/18046 | 0.000464512 | 0.01242854 | 8021/23636/ |
| OF_GENERATION_OF_ | | | | | 53371/ |
| PRECURSOR_METABOLITES_ | | | | | 4927/9818 |
| AND_ENERGY | | | | | |
| GO_HIPPO_SIGNALING | 3/74 | 38/18046 | 0.000503668 | 0.01315534 | 6789/6788/60485 |
| GO_NEGATIVE_REGULATION_ | 3/74 | 40/18046 | 0.000586424 | 0.014960644 | 6789/6788/60485 |
| OF_ORGAN_GROWTH | | | | | |
| GO_UBIQUITIN_LIKE_ | 4/74 | 96/18046 | 0.000650796 | 0.016225522 | 8924/22954/ |
| PROTEIN_BINDING | | | | | 29761/23636 |
| GO_PROTEIN_TRIMERIZATION | 3/74 | 42/18046 | 0.000677399 | 0.016513494 | 23636/53371/9818 |
| GO_PROTEIN_LOCALIZATION_ | 6/74 | 266/18046 | 0.000776573 | 0.018519573 | 10204/8021/ |
| TO_NUCLEUS | | | | | 23636/53371/ |
| | | | | | 4927/9818 |
| GO_PROTEIN_ | 3/74 | 46/18046 | 0.000885264 | 0.020358488 | 26519/90580/ |
| INSERTION_INTO_ | | | | | 26520 |
| MITOCHONDRIAL_MEMBRANE | | | | | |
| GO_CHAPERONE_MEDIATED_ | 2/74 | 11/18046 | 0.0008908 | 0.020358488 | 26519/26520 |
| PROTEIN_TRANSPORT | | | | | |
| GO_RESPONSE_TO_HEAT | 5/74 | 183/18046 | 0.000928986 | 0.020797914 | 8021/23636/53371/ |
| | | | | | 4927/9818 |
| GO_POSITIVE_REGULATION_ | 5/74 | 184/18046 | 0.00095192 | 0.020885115 | 23476/57153/22954/ |
| OF_I_KAPPAB_KINASE_NF_ | | | | | 29110/23636 |
| KAPPAB_SIGNALING | | | | | |
| GO_HEPATOCYTE_APOPTOTIC_ | 2/74 | 12/18046 | 0.001066124 | 0.022932111 | 6789/6788 |
| PROCESS | | | | | |
| GO_NEGATIVE_REGULATION_ | 4/74 | 111/18046 | 0.001120296 | 0.02363394 | 10505/6789/6788/ |
| OF_DEVELOPMENTAL_ | | | | | 60485 |
| GROWTH | | | | | |
| GO_MITOCHONDRIAL_ | 2/74 | 13/18046 | 0.001256622 | 0.026009714 | 9512/23203 |
| PROTEIN_PROCESSING | | | | | |
| GO_CARBOHYDRATE_ | 5/74 | 198/18046 | 0.001319193 | 0.026799156 | 8021/23636/53371/ |
| CATABOLIC_PROCESS | | | | | 4927/9818 |
| GO_REGULATION_OF_PROTEIN_ | 4/74 | 119/18046 | 0.001449261 | 0.028140401 | 10204/23636/ |
| LOCALIZATION_TO_NUCLEUS | | | | | 53371/9818 |
| GO_ENDOCARDIUM_ | 2/74 | 14/18046 | 0.001462172 | 0.028140401 | 6789/6788 |
| DEVELOPMENT | | | | | |
| GO_POSITIVE_REGULATION_ | 2/74 | 14/18046 | 0.001462172 | 0.028140401 | 6789/6788 |
| OF_EXTRINSIC_APOPTOTIC_ | | | | | |
| SIGNALING_PATHWAY_VIA_ | | | | | |
| DEATH_DOMAIN_RECEPTORS | | | | | |
| GO_REGULATION_ | 5/74 | 204/18046 | 0.00150506 | 0.028466402 | 8021/23636/ |
| OF_CARBOHYDRATE_ | | | | | 53371/ |
| METABOLIC_PROCESS | | | | | 4927/9818 |
| GO_PROTEIN_INSERTION_ | 3/74 | 62/18046 | 0.002104496 | 0.03912935 | 26519/90580/ |
| INTO_MEMBRANE | | | | | 26520 |
| GO_VIRAL_LIFE_CYCLE | 6/74 | 328/18046 | 0.002261425 | 0.040133817 | 22954/8021/ |
| | | | | | 23636/ |
| | | | | | 53371/4927/ |
| | | | | | 9818 |
| GO_INNER_MITOCHONDRIAL_ | 4/74 | 135/18046 | 0.002299199 | 0.040133817 | 26519/90580/ |
| MEMBRANE_PROTEIN_ | | | | | 55735/26515 |
| COMPLEX | | | | | |
| GO_MITOCHONDRIAL_ | 4/74 | 135/18046 | 0.002299199 | 0.040133817 | 26519/90580/ |
| MEMBRANE_ | | | | | 55735/26520 |
| ORGANIZATION | | | | | |
| GO_POSITIVE_REGULATION_ | 3/74 | 64/18046 | 0.002304859 | 0.040133817 | 6789/6788/60485 |
| OF_FAT_CELL_ | | | | | |
| DIFFERENTIATION | | | | | |

TABLE 9G
MERS

| Description | GeneRatio | BgRatio | pvalue | p adjust | geneID |
|---|---|---|---|---|---|
| GO_RIBOSOME_BIOGENESIS | 37/289 | 290/18046 | 8.90E−23 | 2.93E−19 | 55127/11340/4931/ |
| | | | | | 9875/10775/23517/ |
| | | | | | 10153/10607/1662/ |
| | | | | | 9816/5822/55035/55027/ |
| | | | | | 134430/10200/79954/ |
| | | | | | 55759/65083/27341/ |
| | | | | | 29889/23212/117246/ |
| | | | | | 55661/10969/26574/ |
| | | | | | 51013/10199/9136/ |
| | | | | | 79066/57647/88745/ |
| | | | | | 92856/51187/51116/ |
| | | | | | 51118/65003/708 |
| GO_RIBONUCLEOPROTEIN_ | 41/289 | 419/18046 | 9.47E−21 | 1.56E−17 | 55127/11340/8663/ |
| COMPLEX_BIOGENESIS | | | | | 10480/4931/9875/10775/ |
| | | | | | 23517/10153/10607/ |
| | | | | | 1662/9816/5822/ |
| | | | | | 55035/55027/134430/ |
| | | | | | 10200/79954/55759/ |
| | | | | | 65083/27341/29889/ |
| | | | | | 23212/117246/55661/ |
| | | | | | 10969/26574/51013/ |
| | | | | | 10199/9136/79066/ |
| | | | | | 57647/88745/92856/ |
| | | | | | 96764/51187/23405/ |
| | | | | | 51116/51118/65003/ |
| | | | | | 708 |
| GO_RRNA_METABOLIC_ | 29/289 | 221/18046 | 2.19E−18 | 2.40E−15 | 55127/115752/11340/ |
| PROCESS | | | | | 4931/9875/10775/ |
| | | | | | 23517/10607/1662/ |
| | | | | | 5822/55035/134430/ |
| | | | | | 10200/79954/55759/ |
| | | | | | 65083/27341/23212/ |
| | | | | | 117246/55661/10969/ |
| | | | | | 51013/10199/9136/ |
| | | | | | 79066/57647/88745/ |
| | | | | | 92856/51118 |
| GO_NCRNA_PROCESSING | 33/289 | 378/18046 | 2.16E−15 | 1.78E−12 | 55127/4087/11340/ |
| | | | | | 4931/9875/10775/ |
| | | | | | 23517/10607/1662/5822/ |
| | | | | | 55035/134430/10200/ |
| | | | | | 79954/55759/65083/ |
| | | | | | 27341/23212/117246/ |
| | | | | | 55661/10969/51013/ |
| | | | | | 10199/9136/8575/ |
| | | | | | 79670/79066/57647/ |
| | | | | | 88745/92856/81890/ |
| | | | | | 23405/51118 |
| GO_NCRNA_METABOLIC_ | 36/289 | 471/18046 | 6.72E−15 | 4.42E−12 | 55127/4087/115752/ |
| PROCESS | | | | | 11340/4931/9875/ |
| | | | | | 10775/23517/10607/ |
| | | | | | 1662/5822/55035/134430/ |
| | | | | | 10200/79954/55759/ |
| | | | | | 65083/27341/23212/ |
| | | | | | 117246/55661/ |
| | | | | | 10969/51013/2617/ |
| | | | | | 10199/9136/8575/79670/ |
| | | | | | 56257/79066/57647/ |
| | | | | | 88745/92856/81890/ |
| | | | | | 23405/51118 |
| GO_PRERIBOSOME | 16/289 | 77/18046 | 7.03E−14 | 3.85E−11 | 55127/10607/5822/ |
| | | | | | 134430/79954/55759/ |
| | | | | | 65083/27341/23212/ |
| | | | | | 117246/10969/10199/ |
| | | | | | 9136/88745/92856/ |
| | | | | | 51118 |
| GO_90S_PRERIBOSOME | 10/289 | 32/18046 | 4.49E−11 | 2.11E−08 | 55127/10607/5822/ |
| | | | | | 134430/55759/65083/ |
| | | | | | 27341/10199/88745/ |
| | | | | | 92856 |
| GO_RIBONUCLEOPROTEIN_ | 16/289 | 130/18046 | 2.92E−10 | 1.04E−07 | 26046/10985/2475/ |
| COMPLEX_BINDING | | | | | 85451/25875/4931/ |
| | | | | | 55759/29789/4830/ |
| | | | | | 6731/6728/3508/6729/ |
| | | | | | 23107/708/7917 |
| GO_SMALL_SUBUNIT_ | 10/289 | 38/18046 | 3.02E−10 | 1.04E−07 | 55127/10607/5822/ |
| PROCESSOME | | | | | 134430/79954/65083/ |
| | | | | | 10199/9136/92856/ |
| | | | | | 51118 |
| GO_MITOCHONDRIAL_ | 9/289 | 28/18046 | 3.24E−10 | 1.04E−07 | 7818/64969/23107/ |
| SMALL_RIBOSOMAL_ | | | | | 64951/51650/28957/ |
| SUBUNIT | | | | | 51116/64960/64965 |
| GO_PROTEIN_ | 22/289 | 266/18046 | 3.48E−10 | 1.04E−07 | 23534/51194/6774/ |
| LOCALIZATION_TO_ | | | | | 7704/51366/7157/ |
| NUCLEUS | | | | | 163590/51512/4931/ |
| | | | | | 10527/55035/55027/ |
| | | | | | 23212/5594/3839/3840/ |
| | | | | | 3841/23633/9972/ |
| | | | | | 3838/3836/10762 |
| GO_NUCLEAR_IMPORT_ | 8/289 | 20/18046 | 4.19E−10 | 1.15E−07 | 23534/51194/3839/ |
| SIGNAL_RECEPTOR_ | | | | | 3840/3841/23633/ |
| ACTIVITY | | | | | 3838/3836 |
| GO_CELL_CYCLE_G2_M_ | 22/289 | 271/18046 | 4.96E−10 | 1.25E−07 | 5116/5566/5577/1063/ |
| PHASE_TRANSITION | | | | | 5108/9662/55755/ |
| | | | | | 10142/11190/22981/ |
| | | | | | 22994/7157/5518/ |
| | | | | | 4361/5520/9113/5704/ |
| | | | | | 55722/121441/51512/ |
| | | | | | 26993/5715 |
| GO_REGULATION_OF_CELL_ | 19/289 | 214/18046 | 1.77E−09 | 4.15E−07 | 5116/5566/5577/1063/ |
| CYCLE_G2_M_PHASE_ | | | | | 5108/9662/55755/ |
| TRANSITION | | | | | 10142/11190/22981/ |
| | | | | | 22994/7157/5518/ |
| | | | | | 4361/5704/55722/ |
| | | | | | 121441/51512/5715 |
| GO_CILIARY_BASAL_BODY_ | 13/289 | 95/18046 | 3.76E−09 | 8.25E−07 | 5116/5566/5577/5108/ |
| PLASMA_MEMBRANE_ | | | | | 9662/55755/10142/ |
| DOCKING | | | | | 11190/22981/22994/ |
| | | | | | 5518/55722/121441 |
| GO_IMPORT_INTO_NUCLEUS | 16/289 | 164/18046 | 9.07E−09 | 1.86E−06 | 23534/51194/6774/ |
| | | | | | 51366/7157/10527/ |
| | | | | | 55027/5594/3839/3840/ |
| | | | | | 3841/23633/9972/ |
| | | | | | 3838/3836/10762 |
| GO_TRANSLATIONAL_ | 13/289 | 105/18046 | 1.30E−08 | 2.52E−06 | 2935/7818/64432/ |
| TERMINATION | | | | | 64969/23107/64951/ |
| | | | | | 29088/51650/28957/ |
| | | | | | 51116/64960/64965/ |
| | | | | | 65003 |
| GO_MITOCHONDRIAL_ | 12/289 | 89/18046 | 1.79E−08 | 3.28E−06 | 7818/64432/64969/ |
| TRANSLATIONAL_ | | | | | 23107/64951/29088/ |
| TERMINATION | | | | | 51650/28957/51116/ |
| | | | | | 64960/64965/65003 |
| GO_RIBOSOME_BINDING | 10/289 | 57/18046 | 2.11E−08 | 3.66E−06 | 10985/2475/25875/ |
| | | | | | 29789/6731/6728/3508/ |
| | | | | | 23107/708/7917 |
| GO_NUCLEOCYTOPLASMIC_ | 8/289 | 31/18046 | 2.25E−08 | 3.70E−06 | 23534/51194/3839/ |
| CARRIER_ACTIVITY | | | | | 3840/3841/23633/ |
| | | | | | 3838/3836 |
| GO_MEMBRANE_DOCKING | 16/289 | 179/18046 | 3.17E−08 | 4.96E−06 | 23256/8673/5116/ |
| | | | | | 5566/5577/5108/9662/ |
| | | | | | 55755/10142/11190/ |
| | | | | | 22981/22994/5518/ |
| | | | | | 55722/6814/121441 |
| GO_MITOCHONDRIAL_ | 14/289 | 137/18046 | 4.33E−08 | 6.48E−06 | 2617/7818/64432/ |
| TRANSLATION | | | | | 64969/23107/64951/ |
| | | | | | 29088/51650/28957/ |
| | | | | | 51116/64960/64965/ |
| | | | | | 65003/708 |
| GO_NUCLEAR_TRANSPORT | 22/289 | 347/18046 | 4.66E−08 | 6.67E−06 | 23534/23225/51194/ |
| | | | | | 6774/5566/51366/ |
| | | | | | 7157/51512/10527/ |
| | | | | | 55027/65083/26993/ |
| | | | | | 23212/5594/3839/3840/ |
| | | | | | 3841/23633/9972/ |
| | | | | | 3838/3836/10762 |
| GO_PROTEIN_IMPORT | 16/289 | 192/18046 | 8.47E−08 | 1.16E−05 | 23534/51194/6774/ |
| | | | | | 51366/7157/10527/ |
| | | | | | 55027/5594/3839/3840/ |
| | | | | | 3841/23633/9972/ |
| | | | | | 3838/3836/10762 |
| GO_MATURATION_OF_5_8S_ | 7/289 | 26/18046 | 1.27E−07 | 1.68E−05 | 11340/9875/23517/ |
| RRNA | | | | | 10200/55759/23212/ |
| | | | | | 117246 |
| GO_ORGANELLAR_ | 11/289 | 87/18046 | 1.40E−07 | 1.77E−05 | 7818/64969/23107/ |
| RIBOSOME | | | | | 64951/29088/51650/ |
| | | | | | 28957/51116/64960/ |
| | | | | | 64965/65003 |
| GO_NUCLEAR_ | 7/289 | 27/18046 | 1.70E−07 | 2.07E−05 | 3839/3840/3841/23633/ |
| LOCALIZATION_SEQUENCE_ | | | | | 9972/3838/3836 |
| BINDING | | | | | |
| GO_TRANSLATIONAL_ | 13/289 | 135/18046 | 2.66E−07 | 3.13E−05 | 26046/7818/64432/ |
| ELONGATION | | | | | 64969/23107/64951/ |
| | | | | | 29088/51650/28957/ |
| | | | | | 51116/64960/64965/ |
| | | | | | 65003 |
| GO_MITOCHONDRIAL_GENE_ | 14/289 | 165/18046 | 4.42E−07 | 5.02E−05 | 2617/7818/64432/ |
| EXPRESSION | | | | | 64969/23107/64951/ |
| | | | | | 29088/51650/28957/ |
| | | | | | 51116/64960/64965/ |
| | | | | | 65003/708 |
| GO_NLS_BEARING_PROTEIN_ | 6/289 | 20/18046 | 5.14E−07 | 5.64E−05 | 3839/3840/3841/23633/ |
| IMPORT_INTO_NUCLEUS | | | | | 3838/3836 |
| GO_REGULATION_OF_ | 16/289 | 227/18046 | 8.27E−07 | 8.74E−05 | 1459/5116/5566/5108/ |
| INTRACELLULAR_PROTEIN_ | | | | | 9648/22994/51366/ |
| TRANSPORT | | | | | 7157/10956/27248/ |
| | | | | | 3998/51512/26229/ |
| | | | | | 26993/5594/10055 |
| GO_SIGNAL_SEQUENCE_ | 8/289 | 48/18046 | 8.50E−07 | 8.74E−05 | 6729/3839/3840/3841/ |
| BINDING | | | | | 23633/9972/3838/ |
| | | | | | 3836 |
| GO_REGULATION_OF_ | 20/289 | 348/18046 | 9.18E−07 | 9.14E−05 | 23256/8673/1459/ |
| INTRACELLULAR_ | | | | | 5116/5566/5108/9648/ |
| TRANSPORT | | | | | 22994/51366/7157/ |
| | | | | | 10956/27248/3998/ |
| | | | | | 51512/26229/26993/ |
| | | | | | 5595/5594/10055/9972 |
| GO_MATURATION_OF_SSU_ | 7/289 | 35/18046 | 1.15E−06 | 0.000111298 | 55127/10607/5822/ |
| RRNA_FROM_TRICISTRONIC_ | | | | | 79954/23212/57647/ |
| RRNA_TRANSCRIPT_SSU_ | | | | | 88745 |
| RRNA_5_8S_RRNA_LSU_RRNA | | | | | |
| GO_RIBOSOMAL_LARGE_ | 9/289 | 68/18046 | 1.32E−06 | 0.000123902 | 4931/9875/55027/ |
| SUBUNIT_BIOGENESIS | | | | | 55759/23212/117246/ |
| | | | | | 10969/51187/65003 |
| GO_NUCLEAR_PORE | 10/289 | 92/18046 | 2.15E−06 | 0.0001967 | 23225/10527/3839/ |
| | | | | | 3840/3841/23633/9972/ |
| | | | | | 3838/3836/10762 |
| GO_SMALL_RIBOSOMAL_ | 9/289 | 73/18046 | 2.42E−06 | 0.000215305 | 7818/64969/23107/ |
| SUBUNIT | | | | | 64951/51650/28957/ |
| | | | | | 51116/64960/64965 |
| GO_EXORIBONUCLEASE_ | 6/289 | 26/18046 | 2.82E−06 | 0.000243727 | 115752/11340/4931/ |
| COMPLEX | | | | | 23517/10200/51013 |
| GO_REGULATION_OF_CELL_ | 23/289 | 482/18046 | 3.40E−06 | 0.000286405 | 5116/5566/5577/1063/ |
| CYCLE_PHASE_TRANSITION | | | | | 5108/9662/55755/ |
| | | | | | 10142/11190/22981/ |
| | | | | | 22994/7157/5518/ |
| | | | | | 4361/5704/55722/26058/ |
| | | | | | 2071/121441/51512/ |
| | | | | | 1642/5715/56257 |
| GO_NUCLEAR_EXOSOME_ | 5/289 | 16/18046 | 3.85E−06 | 0.000316248 | 11340/4931/23517/ |
| RNASE_COMPLEX | | | | | 10200/51013 |
| GO_RIBOSOME | 15/289 | 228/18046 | 4.29E−06 | 0.000344037 | 10985/9513/7818/ |
| | | | | | 64432/64969/23107/ |
| | | | | | 64951/29088/51187/ |
| | | | | | 51650/28957/51116/ |
| | | | | | 64960/64965/65003 |
| GO_MODULATION_BY_ | 5/289 | 18/18046 | 7.35E−06 | 0.000575455 | 3839/3840/3841/ |
| VIRUS_OF_HOST_CELLULAR_ | | | | | 3838/3836 |
| PROCESS | | | | | |
| GO_RNA_CATABOLIC_ | 20/289 | 404/18046 | 8.78E−06 | 0.000671283 | 55802/2475/5518/ |
| PROCESS | | | | | 5520/5704/26058/115752/ |
| | | | | | 11340/23112/2935/ |
| | | | | | 23517/8087/9513/ |
| | | | | | 51013/5715/27258/ |
| | | | | | 6050/79670/79066/ |
| | | | | | 246243 |
| GO_MATURATION_OF_SSU_ | 7/289 | 47/18046 | 9.13E−06 | 0.000682503 | 55127/10607/5822/ |
| RRNA | | | | | 79954/23212/57647/ |
| | | | | | 88745 |
| GO_RIBOSOMAL_SUBUNIT | 13/289 | 186/18046 | 9.82E−06 | 0.000700525 | 9513/7818/64969/ |
| | | | | | 23107/64951/29088/ |
| | | | | | 51187/51650/28957/ |
| | | | | | 51116/64960/64965/ |
| | | | | | 65003 |
| GO_MICROTUBULE_ | 11/289 | 133/18046 | 9.83E−06 | 0.000700525 | 2801/5108/9662/9648/ |
| ORGANIZING_CENTER_ | | | | | 51199/55755/11190/ |
| ORGANIZATION | | | | | 55968/22994/55722/ |
| | | | | | 79884 |
| GO_MODULATION_BY_VIRUS | 6/289 | 32/18046 | 1.02E−05 | 0.000700525 | 3839/3840/3841/23633/ |
| OF_HOST_MORPHOLOGY_ | | | | | 3838/3836 |
| OR_PHYSIOLOGY | | | | | |
| GO_PROTEIN_LOCALIZATION_ | 6/289 | 32/18046 | 1.02E−05 | 0.000700525 | 5108/11190/55968/ |
| TO_MICROTUBULE_ | | | | | 22994/55722/121441 |
| ORGANIZING_CENTER | | | | | |
| GO_PROTEIN_KINASE_A_ | 7/289 | 49/18046 | 1.21E−05 | 0.00081418 | 5576/5566/5577/10142/ |
| BINDING | | | | | 5573/26993/8227 |
| GO_CAMP_DEPENDENT_ | 4/289 | 10/18046 | 1.25E−05 | 0.00081418 | 5576/5566/5577/5573 |
| PROTEIN_KINASE_COMPLEX | | | | | |
| GO_RIBOSOMAL_SMALL_ | 8/289 | 68/18046 | 1.26E−05 | 0.00081418 | 55127/10607/5822/ |
| SUBUNIT_BIOGENESIS | | | | | 79954/27341/23212/ |
| | | | | | 57647/88745 |
| GO_MATURATION_OF_5_8S_ | 5/289 | 21/18046 | 1.68E−05 | 0.001061202 | 11340/9875/55759/ |
| RRNA_FROM_TRICISTRONIC_ | | | | | 23212/117246 |
| RRNA_TRANSCRIPT_SSU_ | | | | | |
| RRNA_5_8S_RRNA_LSU_RRNA | | | | | |
| GO_HOST_CELLULAR_ | 8/289 | 75/18046 | 2.62E−05 | 0.00162323 | 23225/3998/3839/ |
| COMPONENT | | | | | 23633/9972/3838/3836/ |
| | | | | | 10762 |
| GO_POSITIVE_REGULATION_ | 11/289 | 153/18046 | 3.67E−05 | 0.002232359 | 1459/5116/5566/5108/ |
| OF_INTRACELLULAR_ | | | | | 22994/51366/7157/ |
| PROTEIN_TRANSPORT | | | | | 51512/26229/5594/ |
| | | | | | 10055 |
| GO_PROTEIN_LOCALIZATION_ | 7/289 | 58/18046 | 3.76E−05 | 0.00224621 | 5108/11190/55968/ |
| TO_CYTOSKELETON | | | | | 22994/55722/121441/ |
| | | | | | 6242 |
| GO_CATALYTIC_ACTIVITY_ | 18/289 | 380/18046 | 4.42E−05 | 0.00259851 | 115752/10775/23517/ |
| ACTING_ON_RNA | | | | | 1662/117246/55661/ |
| | | | | | 2617/3508/79670/ |
| | | | | | 56257/79066/57647/ |
| | | | | | 27037/96764/81890/ |
| | | | | | 64848/23405/246243 |
| GO_HELICASE_ACTIVITY | 11/289 | 157/18046 | 4.65E−05 | 0.002680741 | 4361/10111/10973/ |
| | | | | | 2071/23517/1662/55661/ |
| | | | | | 3508/57647/64848/ |
| | | | | | 23405 |
| GO_MODULATION_BY_ | 5/289 | 26/18046 | 5.08E−05 | 0.002880233 | 3839/3840/3841/3838/ |
| SYMBIONT_OF_HOST_ | | | | | 3836 |
| CELLULAR_PROCESS | | | | | |
| GO_CELLULAR_PROTEIN_ | 13/289 | 219/18046 | 5.49E−05 | 0.003057822 | 2935/7818/64432/ |
| COMPLEX_DISASSEMBLY | | | | | 64969/23107/64951/ |
| | | | | | 29088/51650/28957/ |
| | | | | | 51116/64960/64965/ |
| | | | | | 65003 |
| GO_MULTI_ORGANISM_ | 7/289 | 62/18046 | 5.82E−05 | 0.003136764 | 23225/3839/23633/ |
| LOCALIZATION | | | | | 9972/3838/3836/10762 |
| GO_RIBOSOME_ASSEMBLY | 7/289 | 62/18046 | 5.82E−05 | 0.003136764 | 5822/27341/23212/ |
| | | | | | 51187/51116/65003/ |
| | | | | | 708 |
| GO_MODIFICATION_BY_ | 6/289 | 43/18046 | 5.93E−05 | 0.003147448 | 3839/3840/3841/ |
| SYMBIONT_OF_HOST_ | | | | | 23633/3838/3836 |
| MORPHOLOGY_OR_ | | | | | |
| PHYSIOLOGY | | | | | |
| GO_STRUCTURAL_ | 11/289 | 162/18046 | 6.18E−05 | 0.003227572 | 7818/64432/64969/ |
| CONSTITUENT_OF_RIBOSOME | | | | | 64951/29088/51187/ |
| | | | | | 51650/51116/64960/ |
| | | | | | 64965/65003 |
| GO_REGULATION_OF_GOLGI_ | 4/289 | 15/18046 | 7.65E−05 | 0.003911288 | 9659/10142/5595/5594 |
| ORGANIZATION | | | | | |
| GO_MITOCHONDRIAL_ | 20/289 | 471/18046 | 7.73E−05 | 0.003911288 | 79586/33/7157/23597/ |
| MATRIX | | | | | 4833/5163/2617/501/ |
| | | | | | 7818/64969/23107/ |
| | | | | | 64951/29088/51650/ |
| | | | | | 28957/51116/64960/ |
| | | | | | 64965/65003/708 |
| GO_MITOCHONDRIAL_ | 14/289 | 260/18046 | 8.20E−05 | 0.004086201 | 5163/55750/26520/ |
| PROTEIN_COMPLEX | | | | | 7818/64969/23107/ |
| | | | | | 64951/29088/51650/ |
| | | | | | 28957/51116/64960/ |
| | | | | | 64965/65003 |
| GO_ATPASE_ACTIVITY | 19/289 | 438/18046 | 8.83E−05 | 0.004336726 | 4643/4627/4361/10111/ |
| | | | | | 23078/5704/10973/ |
| | | | | | 84896/57130/2071/ |
| | | | | | 4931/23517/1662/ |
| | | | | | 29789/55661/3508/ |
| | | | | | 57647/64848/23405 |
| GO_GOLGI_ORGANIZATION | 10/289 | 142/18046 | 9.83E−05 | 0.004755113 | 25923/81562/2801/ |
| | | | | | 9659/9648/10142/ |
| | | | | | 55968/3998/5595/5594 |
| GO_OUTER_MEMBRANE | 12/289 | 204/18046 | 0.000115307 | 0.005496314 | 140707/54708/2475/ |
| | | | | | 1727/64757/51566/ |
| | | | | | 2181/55750/25875/ |
| | | | | | 23098/4830/81890 |
| GO_AMINO_ACID_BETAINE_ | 4/289 | 17/18046 | 0.000130061 | 0.006111016 | 33/5447/501/223 |
| METABOLIC_PROCESS | | | | | |
| GO_POSITIVE_REGULATION_ | 12/289 | 207/18046 | 0.000132325 | 0.006129826 | 8673/1459/5116/5566/ |
| OF_INTRACELLULAR_ | | | | | 5108/22994/51366/ |
| TRANSPORT | | | | | 7157/51512/26229/ |
| | | | | | 5594/10055 |
| GO_ACTIVATION_OF_ | 4/289 | 18/18046 | 0.000165122 | 0.007542861 | 5576/5566/5577/5573 |
| PROTEIN_KINASE_A_ | | | | | |
| ACTIVITY | | | | | |
| GO_ATPASE_ACTIVITY_ | 14/289 | 286/18046 | 0.000222337 | 0.009935687 | 4643/4627/4361/10111/ |
| COUPLED | | | | | 5704/10973/2071/ |
| | | | | | 23517/1662/55661/ |
| | | | | | 3508/57647/64848/ |
| | | | | | 23405 |
| GO_REGULATION_OF_ | 13/289 | 252/18046 | 0.000223545 | 0.009935687 | 1459/1457/5566/9113/ |
| CELLULAR_PROTEIN_ | | | | | 5704/10956/8975/ |
| CATABOLIC_PROCESS | | | | | 27248/25898/5887/ |
| | | | | | 9817/7874/7917 |
| GO_NUCLEAR_ENVELOPE | 19/289 | 472/18046 | 0.000231141 | 0.010136285 | 79188/23225/51194/ |
| | | | | | 1063/5108/7157/3482/ |
| | | | | | 163590/169714/10527/ |
| | | | | | 5595/3839/3840/ |
| | | | | | 3841/23633/9972/ |
| | | | | | 3838/3836/10762 |
| GO_ENDOMEMBRANE_ | 18/289 | 436/18046 | 0.000248416 | 0.010750509 | 25923/79188/81562/ |
| SYSTEM_ORGANIZATION | | | | | 2801/9659/9648/ |
| | | | | | 10142/55968/4627/5518/ |
| | | | | | 5520/3998/163590/ |
| | | | | | 26993/5595/5594/ |
| | | | | | 8266/7917 |
| GO_PROTEIN_CONTAINING_ | 15/289 | 326/18046 | 0.00026092 | 0.011145006 | 8673/79443/2935/ |
| COMPLEX_DISASSEMBLY | | | | | 7818/64432/64969/ |
| | | | | | 23107/64951/29088/ |
| | | | | | 51650/28957/51116/ |
| | | | | | 64960/64965/65003 |
| GO_REGULATION_OF_ | 7/289 | 79/18046 | 0.00027209 | 0.011473122 | 23225/2475/3337/5595/ |
| CELLULAR_RESPONSE_TO_ | | | | | 5594/9972/10762 |
| HEAT | | | | | |
| GO_ENDOPLASMIC_ | 6/289 | 57/18046 | 0.000292804 | 0.012190288 | 25923/81562/3998/ |
| RETICULUM_ | | | | | 163590/8266/7917 |
| ORGANIZATION | | | | | |
| GO_PERICENTRIOLAR_ | 4/289 | 21/18046 | 0.000310958 | 0.012784281 | 5108/51199/55755/ |
| MATERIAL | | | | | 121441 |
| GO_LONG_CHAIN_FATTY_ | 8/289 | 107/18046 | 0.000325199 | 0.013096082 | 33/10005/3295/11001/ |
| ACID_METABOLIC_PROCESS | | | | | 2181/10999/80142/ |
| | | | | | 5595 |
| GO_NUCLEIC_ACID_ | 14/289 | 297/18046 | 0.000326506 | 0.013096082 | 55802/4361/10111/ |
| PHOSPHODIESTER_BOND_ | | | | | 115752/11340/2071/ |
| HYDROLYSIS | | | | | 10775/1642/23212/ |
| | | | | | 51013/88745/23405/ |
| | | | | | 246243/3836 |
| GO_REGULATION_OF_MRNA_ | 11/289 | 199/18046 | 0.000377092 | 0.014942851 | 55802/2475/5704/ |
| CATABOLIC_PROCESS | | | | | 26058/11340/23112/ |
| | | | | | 8087/9513/51013/5715/ |
| | | | | | 79066 |
| GO_PRERIBOSOME_LARGE_ | 4/289 | 23/18046 | 0.000448619 | 0.016772199 | 55759/23212/117246/ |
| SUBUNIT_PRECURSOR | | | | | 10969 |
| GO_CAMP_DEPENDENT_ | 3/289 | 10/18046 | 0.000448754 | 0.016772199 | 5576/5577/5573 |
| PROTEIN_KINASE_ | | | | | |
| REGULATOR_ACTIVITY | | | | | |
| GO_DNA_DEALKYLATION_ | 3/289 | 10/18046 | 0.000448754 | 0.016772199 | 10973/51008/84164 |
| INVOLVED_IN_DNA_REPAIR | | | | | |
| GO_MICROTUBULE_ | 3/289 | 10/18046 | 0.000448754 | 0.016772199 | 5108/51199/22981 |
| ANCHORING_AT_ | | | | | |
| CENTROSOME | | | | | |
| GO_REGULATION_OF_ | 3/289 | 10/18046 | 0.000448754 | 0.016772199 | 11190/55968/55722 |
| PROTEIN_LOCALIZATION_ | | | | | |
| TO_CENTROSOME | | | | | |
| GO_INTERACTION_WITH_ | 11/289 | 204/18046 | 0.000465127 | 0.017181914 | 8673/3956/1642/3839/ |
| HOST | | | | | 3840/3841/23633/ |
| | | | | | 9972/3838/3836/ |
| | | | | | 7037 |
| GO_MODIFICATION_OF_ | 8/289 | 113/18046 | 0.000470165 | 0.017181914 | 3482/64848/3839/3840/ |
| MORPHOLOGY_OR_ | | | | | 3841/23633/3838/ |
| PHYSIOLOGY_OF_OTHER_ | | | | | 3836 |
| ORGANISM_INVOLVED_IN_ | | | | | |
| SYMBIOTIC_INTERACTION | | | | | |
| GO_CELLULAR_RESPONSE_ | 9/289 | 142/18046 | 0.000477016 | 0.017240714 | 23225/2475/5566/ |
| TO_HEAT | | | | | 7157/3337/5595/5594/ |
| | | | | | 9972/10762 |
| GO_DNA_GEOMETRIC_ | 8/289 | 114/18046 | 0.000498758 | 0.017830591 | 7157/4361/10111/ |
| CHANGE | | | | | 10973/2071/1642/5887/ |
| | | | | | 3508 |
| GO_NEGATIVE_REGULATION_ | 3/289 | 11/18046 | 0.000609741 | 0.021563857 | 5576/5577/5573 |
| OF_CAMP_DEPENDENT_ | | | | | |
| PROTEIN_KINASE_ACTIVITY | | | | | |
| GO_GOLGI_STACK | 9/289 | 150/18046 | 0.000709222 | 0.024206774 | 79586/23256/286451/ |
| | | | | | 2530/2802/2801/ |
| | | | | | 10142/55968/2590 |
| GO_CELLULAR_RESPONSE_ | 4/289 | 26/18046 | 0.000729337 | 0.024206774 | 5576/5566/5577/5573 |
| TO_GLUCAGON_STIMULUS | | | | | |
| GO_MICROTUBULE_ | 4/289 | 26/18046 | 0.000729337 | 0.024206774 | 5108/9648/51199/22981 |
| ANCHORING | | | | | |
| GO_MICROTUBULE_ | 4/289 | 26/18046 | 0.000729337 | 0.024206774 | 10844/2801/51199/10142 |
| NUCLEATION | | | | | |
| GO_REGULATION_OF_ | 4/289 | 26/18046 | 0.000729337 | 0.024206774 | 10111/5595/5594/7874 |
| TELOMERE_CAPPING | | | | | |
| GO_PROTEIN_EXIT_FROM_ | 5/289 | 45/18046 | 0.000735986 | 0.024206774 | 9648/10956/27248/ |
| ENDOPLASMIC_RETICULUM | | | | | 6400/55829 |
| GO_PROTEASOMAL_PROTEIN_ | 18/289 | 478/18046 | 0.000735992 | 0.024206774 | 4189/26046/114088/ |
| CATABOLIC_PROCESS | | | | | 5566/55968/5704/ |
| | | | | | 10956/8975/27248/6400/ |
| | | | | | 55829/1642/5715/ |
| | | | | | 25898/5887/9817/ |
| | | | | | 7874/7917 |
| GO_RESPONSE_TO_HEAT | 10/289 | 183/18046 | 0.000754533 | 0.024570882 | 23225/2475/5566/7157/ |
| | | | | | 3337/11080/5595/ |
| | | | | | 5594/9972/10762 |
| GO_MICROTUBULE_ | 3/289 | 12/18046 | 0.000803382 | 0.025905145 | 5108/51199/22981 |
| ANCHORING_AT_ | | | | | |
| MICROTUBULE_ORGANIZING_ | | | | | |
| CENTER | | | | | |
| GO_POSITIVE_REGULATION_ | 14/289 | 326/18046 | 0.000820039 | 0.025942557 | 1459/5116/5566/5108/ |
| OF_CELLULAR_PROTEIN_ | | | | | 11190/22994/51366/ |
| LOCALIZATION | | | | | 7157/51512/26229/ |
| | | | | | 2181/5594/245812/ |
| | | | | | 10055 |
| GO_REGULATION_OF_ | 10/289 | 185/18046 | 0.000820318 | 0.025942557 | 5566/5704/10956/ |
| PROTEASOMAL_PROTEIN_ | | | | | 8975/27248/25898/5887/ |
| CATABOLIC_PROCESS | | | | | 9817/7874/7917 |
| GO_REGULATION_OF_ | 14/289 | 328/18046 | 0.000869901 | 0.027248614 | 9517/23256/1459/6774/ |
| AUTOPHAGY | | | | | 2475/5566/2801/ |
| | | | | | 79443/7157/8975/ |
| | | | | | 526/523/5595/9817 |
| GO_RESPONSE_TO_AMINO_ | 5/289 | 47/18046 | 0.000900302 | 0.027934845 | 10985/2475/79726/5595/ |
| ACID_STARVATION | | | | | 5594 |
| GO_PROTEIN_C_TERMINUS_ | 10/289 | 189/18046 | 0.000966074 | 0.029517111 | 7704/1063/9662/11190/ |
| BINDING | | | | | 7157/4361/2071/ |
| | | | | | 10055/3839/7874 |
| GO_ENDOPLASMIC_ | 4/289 | 28/18046 | 0.000974071 | 0.029517111 | 10956/27248/6400/ |
| RETICULUM_TO_CYTOSOL_ | | | | | 55829 |
| TRANSPORT | | | | | |
| GO_MACROAUTOPHAGY | 13/289 | 295/18046 | 0.000988285 | 0.029517111 | 9517/23256/8673/1459/ |
| | | | | | 1457/2475/5566/ |
| | | | | | 79443/55968/7157/ |
| | | | | | 526/523/5595 |
| GO_NEGATIVE_REGULATION_ | 12/289 | 259/18046 | 0.000999869 | 0.029517111 | 23256/1459/1457/6774/ |
| OF_CELLULAR_CATABOLIC_ | | | | | 2475/2801/7157/ |
| PROCESS | | | | | 10956/27248/79066/ |
| | | | | | 7874/7917 |
| GO_CARNITINE_METABOLIC_ | 3/289 | 13/18046 | 0.001032067 | 0.029517111 | 33/5447/223 |
| PROCESS | | | | | |
| GO_CENTRIOLE_CENTRIOLE_ | 3/289 | 13/18046 | 0.001032067 | 0.029517111 | 9662/51199/11190 |
| COHESION | | | | | |
| GO_LONG_CHAIN_FATTY_ | 3/289 | 13/18046 | 0.001032067 | 0.029517111 | 11001/2181/10999 |
| ACID_COA_LIGASE_ACTIVITY | | | | | |
| GO_MEIOTIC_SPINDLE_ | 3/289 | 13/18046 | 0.001032067 | 0.029517111 | 2801/4627/5518 |
| ORGANIZATION | | | | | |
| GO_PROTEIN_KINASE_A_ | 3/289 | 13/18046 | 0.001032067 | 0.029517111 | 5576/5577/5573 |
| CATALYTIC_SUBUNIT_ | | | | | |
| BINDING | | | | | |
| GO_ERAD_PATHWAY | 7/289 | 99/18046 | 0.001065618 | 0.030213958 | 4189/10956/8975/27248/ |
| | | | | | 6400/55829/7917 |
| GO_NEGATIVE_REGULATION_ | 6/289 | 73/18046 | 0.001109729 | 0.030827086 | 1459/1457/10956/27248/ |
| OF_PROTEOLYSIS_INVOLVED_ | | | | | 7874/7917 |
| IN_CELLULAR_PROTEIN_ | | | | | |
| CATABOLIC_PROCESS | | | | | |
| GO_PROTEIN_ | 4/289 | 29/18046 | 0.001115817 | 0.030827086 | 10956/55768/6400/23324 |
| DEGLYCOSYLATION | | | | | |
| GO_REGULATION_OF_ERAD_ | 4/289 | 29/18046 | 0.001115817 | 0.030827086 | 10956/8975/27248/7917 |
| PATHWAY | | | | | |
| GO_POSITIVE_REGULATION_ | 8/289 | 129/18046 | 0.001124734 | 0.030827086 | 2475/8663/8087/9513/ |
| OF_TRANSLATION | | | | | 5595/5594/23107/ |
| | | | | | 708 |
| GO_DOUBLE_STRANDED_ | 6/289 | 74/18046 | 0.001191694 | 0.03204572 | 8087/8575/51663/23567/ |
| RNA_BINDING | | | | | 23405/7037 |
| GO_PRODUCTION_OF_SMALL_ | 5/289 | 50/18046 | 0.001195976 | 0.03204572 | 7157/4087/8575/79670/ |
| RNA_INVOLVED_IN_GENE_ | | | | | 23405 |
| SILENCING_BY_RNA | | | | | |
| GO_PROCESS_UTILIZING_ | 18/289 | 499/18046 | 0.001198426 | 0.03204572 | 9517/23256/8673/1459/ |
| AUTOPHAGIC_MECHANISM | | | | | 1457/6774/2475/ |
| | | | | | 5566/2801/79443/55968/ |
| | | | | | 7157/8975/2011/ |
| | | | | | 526/523/5595/9817 |
| GO_CHAPERONE_BINDING | 7/289 | 102/18046 | 0.001269345 | 0.033668357 | 4189/7157/8975/3337/ |
| | | | | | 11080/26520/8266 |
| GO_MATURATION_OF_LSU_ | 3/289 | 14/18046 | 0.001298044 | 0.033883065 | 9875/55759/117246 |
| RRNA_FROM_TRICISTRONIC_ | | | | | |
| RRNA_TRANSCRIPT_SSU_ | | | | | |
| RRNA_5_8S_RRNA_LSU_RRNA | | | | | |
| GO_ORGANELLE_ | 3/289 | 14/18046 | 0.001298044 | 0.033883065 | 2801/5595/5594 |
| INHERITANCE | | | | | |
| GO_NUCLEAR_ENVELOPE_ | 5/289 | 51/18046 | 0.001308859 | 0.033896351 | 79188/55968/5518/ |
| ORGANIZATION | | | | | 5520/26993 |
| GO_ORGANELLE_ | 15/289 | 382/18046 | 0.001331687 | 0.034218102 | 79971/25923/79586/ |
| SUBCOMPARTMENT | | | | | 23256/286451/2530/ |
| | | | | | 55717/2802/2801/ |
| | | | | | 9648/10142/55968/ |
| | | | | | 3482/2590/6786 |
| GO_MESODERM_ | 6/289 | 76/18046 | 0.001369455 | 0.034647212 | 79971/5566/7296/4087/ |
| MORPHOGENESIS | | | | | 2296/5573 |
| GO_RNA_HELICASE_ACTIVITY | 6/289 | 76/18046 | 0.001369455 | 0.034647212 | 23517/1662/55661/ |
| | | | | | 3508/57647/64848 |
| GO_NUCLEAR_TRANSCRIBED_ | 6/289 | 77/18046 | 0.001465583 | 0.036796197 | 55802/11340/23112/ |
| MRNA_CATABOLIC_PROCESS_ | | | | | 51013/27258/79670 |
| DEADENYLATION_ | | | | | |
| DEPENDENT_DECAY | | | | | |
| GO_REGULATION_OF_ | 7/289 | 105/18046 | 0.00150236 | 0.037433803 | 5566/51366/7157/51512/ |
| NUCLEOCYTOPLASMIC_ | | | | | 26993/5594/9972 |
| TRANSPORT | | | | | |
| GO_UBIQUITIN_DEPENDENT_ | 6/289 | 78/18046 | 0.001566767 | 0.038745089 | 4189/10956/27248/ |
| ERAD_PATHWAY | | | | | 6400/55829/7917 |
| GO_LIPID_IMPORT_INTO_ | 3/289 | 15/18046 | 0.001603429 | 0.038777037 | 11001/2181/10999 |
| CELL | | | | | |
| GO_PRE_MIRNA_PROCESSING | 3/289 | 15/18046 | 0.001603429 | 0.038777037 | 8575/79670/23405 |
| GO_PROTEIN_LOCALIZATION_ | 3/289 | 15/18046 | 0.001603429 | 0.038777037 | 4931/55035/23212 |
| TO_NUCLEOLUS | | | | | |
| GO_DNA_DEALKYLATION | 4/289 | 33/18046 | 0.001828322 | 0.043818276 | 10973/51008/84164/ |
| | | | | | 7874 |
| GO_TELOMERE_CAPPING | 5/289 | 55/18046 | 0.001840252 | 0.043818276 | 4361/10111/5595/5594/ |
| | | | | | 7874 |
| GO_REGULATION_OF_ | 9/289 | 172/18046 | 0.001851852 | 0.043818276 | 9517/23256/2475/5566/ |
| MACROAUTOPHAGY | | | | | 79443/7157/526/ |
| | | | | | 523/5595 |
| GO_TRANSLATION_ | 8/289 | 140/18046 | 0.001895642 | 0.044375357 | 10985/2475/8663/10480/ |
| REGULATOR_ACTIVITY | | | | | 2935/8087/9513/708 |
| GO_STRIATED_MUSCLE_ | 6/289 | 81/18046 | 0.001902379 | 0.044375357 | 205428/6774/1482/2 |
| CELL_PROLIFERATION | | | | | 296/5573/5594 |
| GO_REGULATION_OF_ | 17/289 | 484/18046 | 0.002153723 | 0.049884473 | 26046/10985/6774/ |
| CELLULAR_AMIDE_ | | | | | 2475/85451/26058/ |
| METABOLIC_PROCESS | | | | | 8663/23112/2935/5163/ |
| | | | | | 8087/9513/5595/ |
| | | | | | 5594/79066/23107/708 |

TABLE 9H
SARS-COV-1

| Description | GeneRatio | BgRatio | pvalue | p.adjust | geneID |
|---|---|---|---|---|---|
| GO_EUKARYOTIC_48S_ | 13/356 | 15/18046 | 5.59E−21 | 1.93E−17 | 8665/8667/8666/8669/ |
| PREINITIATION_COMPLEX | | | | | 3646/8661/10480/ |
| | | | | | 8663/27335/51386/ |
| | | | | | 8664/8662/8668 |
| GO_EUKARYOTIC_ | 13/356 | 16/18046 | 2.93E−20 | 3.37E−17 | 8665/8667/8666/8669/ |
| TRANSLATION_INITIATION_ | | | | | 3646/8661/10480/ |
| FACTOR_3_COMPLEX | | | | | 8663/27335/51386/ |
| | | | | | 8664/8662/8668 |
| GO_FORMATION_OF_ | 13/356 | 16/18046 | 2.93E−20 | 3.37E−17 | 8665/8667/8666/8669/ |
| CYTOPLASMIC_ | | | | | 3646/8661/10480/ |
| TRANSLATION_INITIATION_ | | | | | 8663/27335/51386/ |
| COMPLEX | | | | | 8664/8662/8668 |
| GO_TRANSLATION_ | 13/356 | 18/18046 | 4.32E−19 | 3.74E−16 | 8665/8667/8666/8669/ |
| PREINITIATION_COMPLEX | | | | | 3646/8661/10480/ |
| | | | | | 8663/27335/51386/ |
| | | | | | 8664/8662/8668 |
| GO_CYTOPLASMIC_ | 14/356 | 31/18046 | 2.05E−16 | 1.42E−13 | 8665/8667/8666/8669/ |
| TRANSLATIONAL_ | | | | | 3646/8661/10480/ |
| INITIATION | | | | | 8663/27335/51386/ |
| | | | | | 8664/8662/8668/2475 |
| GO_TRANSLATION_ | 16/356 | 51/18046 | 1.44E−15 | 8.31E−13 | 8665/8667/9470/8666/ |
| INITIATION_FACTOR_ | | | | | 8669/3646/8661/ |
| ACTIVITY | | | | | 10480/8663/27335/ |
| | | | | | 51386/8664/8662/ |
| | | | | | 8668/1967/4528 |
| GO_TRANSLATION_ | 19/356 | 109/18046 | 3.98E−13 | 1.97E−10 | 23367/26986/8665/ |
| REGULATOR_ACTIVITY_ | | | | | 8667/9470/8666/8669/ |
| NUCLEIC_ACID_BINDING | | | | | 3646/8661/10480/ |
| | | | | | 8663/27335/51386/ |
| | | | | | 8664/8662/8668/1967/ |
| | | | | | 10985/4528 |
| GO_TRANSLATION_FACTOR_ | 17/356 | 85/18046 | 6.70E−13 | 2.90E−10 | 8665/8667/9470/8666/ |
| ACTIVITY_RNA_BINDING | | | | | 8669/3646/8661/ |
| | | | | | 10480/8663/27335/ |
| | | | | | 51386/8664/8662/ |
| | | | | | 8668/1967/10985/4528 |
| GO_TRANSLATION_ | 20/356 | 140/18046 | 4.50E−12 | 1.73E−09 | 23367/26986/8665/ |
| REGULATOR_ACTIVITY | | | | | 8667/9470/8666/8669/ |
| | | | | | 3646/8661/10480/ |
| | | | | | 8663/27335/51386/ |
| | | | | | 8664/8662/8668/1967/ |
| | | | | | 2475/10985/4528 |
| GO_RIBONUCLEOPROTEIN_ | 32/356 | 419/18046 | 6.55E−11 | 2.26E−08 | 55127/9136/6838/ |
| COMPLEX_BIOGENESIS | | | | | 26156/10569/8665/ |
| | | | | | 8667/8666/8669/3646/ |
| | | | | | 8661/10480/8663/27335/ |
| | | | | | 51386/8664/8662/ |
| | | | | | 8668/10199/1662/ |
| | | | | | 9790/57647/11340/ |
| | | | | | 79954/26574/25983/ |
| | | | | | 56915/51010/65003/ |
| | | | | | 27340/55027/23195 |
| GO_CYTOPLASMIC_ | 16/356 | 99/18046 | 9.63E−11 | 3.03E−08 | 8531/25873/8665/8667/ |
| TRANSLATION | | | | | 8666/8669/3646/ |
| | | | | | 8661/10480/8663/ |
| | | | | | 27335/51386/8664/ |
| | | | | | 8662/8668/2475 |
| GO_TRANSLATIONAL_ | 20/356 | 192/18046 | 1.46E−09 | 4.22E−07 | 23367/26986/25873/ |
| INITIATION | | | | | 8665/8667/9470/8666/ |
| | | | | | 8669/3646/8661/ |
| | | | | | 10480/8663/27335/ |
| | | | | | 51386/8664/8662/ |
| | | | | | 8668/1967/2475/4528 |
| GO_ENDOPLASMIC_ | 16/356 | 129/18046 | 5.31E−09 | 1.41E−06 | 10945/90522/26958/ |
| RETICULUM_GOLGI_ | | | | | 57222/2801/2804/ |
| INTERMEDIATE_ | | | | | 399687/64689/10960/ |
| COMPARTMENT | | | | | 126003/23392/22820/ |
| | | | | | 5034/811/23071/56886 |
| GO_CENTRIOLE | 16/356 | 141/18046 | 1.94E−08 | 4.78E−06 | 10426/80184/1070/ |
| | | | | | 9738/54535/219844/ |
| | | | | | 5116/11116/5108/9857/ |
| | | | | | 9662/11190/51199/ |
| | | | | | 8924/84461/4218 |
| GO_CILIARY_BASAL_BODY_ | 13/356 | 95/18046 | 4.48E−08 | 1.03E−05 | 1781/80184/9738/ |
| PLASMA_MEMBRANE_ | | | | | 5116/11116/5566/5108/ |
| DOCKING | | | | | 9662/55755/10142/ |
| | | | | | 11190/22994/22981 |
| GO_MYOSIN_COMPLEX | 10/356 | 55/18046 | 1.05E−07 | 2.26E−05 | 140465/4643/79784/ |
| | | | | | 399687/4645/4646/ |
| | | | | | 22998/4644/4627/4649 |
| GO_VIRAL_TRANSLATION | 6/356 | 15/18046 | 2.43E−07 | 4.95E−05 | 8665/8666/8661/51386/ |
| | | | | | 8664/8662 |
| GO_REGULATION_OF_ | 28/356 | 484/18046 | 4.07E−07 | 7.81E−05 | 26046/6774/79072/ |
| CELLULAR_AMIDE_ | | | | | 8531/23367/26986/ |
| METABOLIC_PROCESS | | | | | 4343/23185/57690/ |
| | | | | | 8667/9470/3646/26058/ |
| | | | | | 90850/8663/27335/ |
| | | | | | 8664/8662/64215/ |
| | | | | | 25983/1967/2475/ |
| | | | | | 10985/811/84300/55245/ |
| | | | | | 4528/63935 |
| GO_RIBONUCLEOPROTEIN_ | 16/356 | 193/18046 | 1.49E−06 | 0.000270292 | 10569/8665/8667/8666/ |
| COMPLEX_SUBUNIT_ | | | | | 8669/3646/8661/ |
| ORGANIZATION | | | | | 10480/8663/27335/ |
| | | | | | 51386/8664/8662/ |
| | | | | | 8668/65003/23195 |
| GO_RIBONUCLEOPROTEIN_ | 13/356 | 130/18046 | 1.80E−06 | 0.000311839 | 26046/8531/1460/ |
| COMPLEX_BINDING | | | | | 23367/90850/27335/ |
| | | | | | 25875/6731/6728/2475/ |
| | | | | | 10985/4528/27044 |
| GO_MEMBRANE_DOCKING | 15/356 | 179/18046 | 2.77E−06 | 0.000455315 | 1781/80184/9738/ |
| | | | | | 5116/11116/5566/5108/ |
| | | | | | 9662/55755/10142/ |
| | | | | | 11190/22994/22981/ |
| | | | | | 4218/4905 |
| GO_INCLUSION_BODY | 10/356 | 78/18046 | 3.01E−06 | 0.000468184 | 5663/8106/2876/4928/ |
| | | | | | 9531/5704/9529/ |
| | | | | | 10273/5424/9463 |
| GO_MICROFILAMENT_ | 6/356 | 22/18046 | 3.23E−06 | 0.000468184 | 4643/79784/4645/4646/ |
| MOTOR_ACTIVITY | | | | | 4644/4627 |
| GO_ACTOMYOSIN | 10/356 | 79/18046 | 3.39E−06 | 0.000468184 | 3983/7168/7171/79784/ |
| | | | | | 399687/22998/4644/ |
| | | | | | 4627/9531/2275 |
| GO_REGULATION_OF_ | 10/356 | 79/18046 | 3.39E−06 | 0.000468184 | 3281/23225/4928/8480/ |
| CELLULAR_RESPONSE_ | | | | | 8021/2475/9531/ |
| TO_HEAT | | | | | 26973/9529/53371 |
| GO_GOLGI_VESICLE_ | 22/356 | 367/18046 | 4.14E−06 | 0.000551029 | 10945/1781/90522/ |
| TRANSPORT | | | | | 23041/26958/57222/ |
| | | | | | 1523/2802/2801/2804/ |
| | | | | | 399687/64689/4644/ |
| | | | | | 54520/4218/10960/ |
| | | | | | 126003/2181/10342/ |
| | | | | | 4905/22820/9463 |
| GO_CELLULAR_RESPONSE_ | 13/356 | 142/18046 | 4.85E−06 | 0.000621324 | 3281/10569/5566/ |
| TO_HEAT | | | | | 23225/4928/8480/8021/ |
| | | | | | 2475/9531/26973/ |
| | | | | | 9529/10273/53371 |
| GO_ACTIN_FILAMENT_ | 15/356 | 190/18046 | 5.77E−06 | 0.000711995 | 55219/3983/2934/ |
| BINDING | | | | | 7168/7111/7171/4643/ |
| | | | | | 2314/79784/399687/ |
| | | | | | 4645/4646/4644/ |
| | | | | | 4627/9463 |
| GO_PROTEIN_FOLDING | 16/356 | 220/18046 | 8.08E−06 | 0.000963703 | 267/1459/1460/1457/ |
| | | | | | 53938/64215/5034/ |
| | | | | | 811/5824/30001/23071/ |
| | | | | | 56886/9601/9531/ |
| | | | | | 26973/9529 |
| GO_MICROTUBULE_ | 6/356 | 26/18046 | 9.32E−06 | 0.000993614 | 5195/11116/5108/9857/ |
| ANCHORING | | | | | 51199/22981 |
| GO_MICROTUBULE_ | 6/356 | 26/18046 | 9.32E−06 | 0.000993614 | 10426/10844/2801/ |
| NUCLEATION | | | | | 10142/51199/10048 |
| GO_POSITIVE_REGULATION_ | 12/356 | 129/18046 | 9.54E−06 | 0.000993614 | 79072/8531/23367/ |
| OF_TRANSLATION | | | | | 26986/23185/3646/ |
| | | | | | 8663/8664/2475/84300/ |
| | | | | | 55245/63935 |
| GO_CADHERIN_BINDING | 20/356 | 330/18046 | 9.69E−06 | 0.000993614 | 5663/23367/26156/ |
| | | | | | 90102/10755/5962/ |
| | | | | | 2802/2801/23085/4627/ |
| | | | | | 3646/26058/26136/ |
| | | | | | 9689/28969/10985/ |
| | | | | | 9531/3069/27044/2011 |
| GO_RESPONSE_TO_ | 17/356 | 249/18046 | 9.77E−06 | 0.000993614 | 490/8531/3281/10569/ |
| TEMPERATURE_STIMULUS | | | | | 5566/23225/1967/ |
| | | | | | 4928/8480/8021/2475/ |
| | | | | | 30001/9531/26973/ |
| | | | | | 9529/10273/53371 |
| GO_REGULATION_OF_MRNA_ | 15/356 | 199/18046 | 1.01E−05 | 0.000997691 | 79072/79675/8531/ |
| CATABOLIC_PROCESS | | | | | 8761/23367/26986/ |
| | | | | | 4343/57690/26058/ |
| | | | | | 11340/56915/51010/ |
| | | | | | 8021/2475/5704 |
| GO_OUTER_MEMBRANE | 15/356 | 204/18046 | 1.36E−05 | 0.001305356 | 5663/4580/140707/ |
| | | | | | 10280/1727/64757/ |
| | | | | | 25875/23111/2181/ |
| | | | | | 65991/2475/9868/ |
| | | | | | 54884/55626/51566 |
| GO_RESPONSE_TO_HEAT | 14/356 | 183/18046 | 1.69E−05 | 0.001574582 | 3281/10569/5566/ |
| | | | | | 23225/1967/4928/8480/ |
| | | | | | 8021/2475/9531/ |
| | | | | | 26973/9529/10273/ |
| | | | | | 53371 |
| GO_RIBOSOME_BIOGENESIS | 18/356 | 290/18046 | 1.96E−05 | 0.001741034 | 55127/9136/6838/ |
| | | | | | 26156/10199/1662/9790/ |
| | | | | | 57647/11340/79954/ |
| | | | | | 26574/25983/56915/ |
| | | | | | 51010/65003/27340/ |
| | | | | | 55027/23195 |
| GO_NUCLEAR_TRANSPORT | 20/356 | 347/18046 | 2.01E−05 | 0.001741034 | 10526/9670/6774/5663/ |
| | | | | | 64328/8106/10569/ |
| | | | | | 54535/5566/23225/ |
| | | | | | 51692/4928/8480/ |
| | | | | | 8021/30000/55027/ |
| | | | | | 811/9531/5494/53371 |
| GO_PROTEIN_DISULFIDE_ | 5/356 | 18/18046 | 2.01E−05 | 0.001741034 | 169714/5034/30001/ |
| ISOMERASE_ACTIVITY | | | | | 23071/9601 |
| GO_SNRNA_3_END_ | 6/356 | 30/18046 | 2.25E−05 | 0.00189567 | 25896/11340/56915/ |
| PROCESSING | | | | | 51010/203522/26512 |
| GO_SNRNA_METABOLIC_ | 7/356 | 45/18046 | 2.61E−05 | 0.002147788 | 25896/56257/11340/ |
| PROCESS | | | | | 56915/51010/203522/ |
| | | | | | 26512 |
| GO_IRES_DEPENDENT_ | 4/356 | 10/18046 | 2.85E−05 | 0.002215389 | 8665/8661/8664/8662 |
| VIRAL_TRANSLATIONAL_ | | | | | |
| INITIATION | | | | | |
| GO_UNCONVENTIONAL_ | 4/356 | 10/18046 | 2.85E−05 | 0.002215389 | 140465/4646/4644/ |
| MYOSIN_COMPLEX | | | | | 4649 |
| GO_PROTEIN_IMPORT | 14/356 | 192/18046 | 2.88E−05 | 0.002215389 | 10526/9670/6774/5663/ |
| | | | | | 8504/5195/51025/ |
| | | | | | 4928/8021/30000/ |
| | | | | | 55027/5824/9531/53371 |
| GO_CYTOPLASMIC_STRESS_ | 8/356 | 63/18046 | 3.19E−05 | 0.002396938 | 10146/8761/23367/ |
| GRANULE | | | | | 9908/26986/4343/ |
| | | | | | 23185/26058 |
| GO_NUCLEAR_EXPORT | 14/356 | 195/18046 | 3.42E−05 | 0.002518156 | 64328/8106/10569/ |
| | | | | | 54535/5566/23225/ |
| | | | | | 51692/4928/8480/8021/ |
| | | | | | 811/9531/5494/53371 |
| GO_MATURATION_OF_SSU_ | 6/356 | 35/18046 | 5.66E−05 | 0.004039362 | 55127/9790/57647/ |
| RRNA_FROM_TRICISTRONIC_ | | | | | 79954/25983/27340 |
| RRNA_TRANSCRIPT_SSU_ | | | | | |
| RRNA_5_8S_RRNA_LSU_RRNA | | | | | |
| GO_DNA_POLYMERASE_ | 5/356 | 22/18046 | 5.80E−05 | 0.004039362 | 23649/5422/5557/5558/ |
| COMPLEX | | | | | 5424 |
| GO_PROCESS_UTILIZING_ | 24/356 | 499/18046 | 5.84E−05 | 0.004039362 | 10548/823/6774/5663/ |
| AUTOPHAGIC_MECHANISM | | | | | 1459/1460/23367/ |
| | | | | | 1457/8897/5566/2801/ |
| | | | | | 8975/54472/26073/ |
| | | | | | 4218/65991/2475/ |
| | | | | | 9373/9868/9531/2011/ |
| | | | | | 10273/23557/55626 |
| GO_POSITIVE_REGULATION_ | 12/356 | 156/18046 | 6.37E−05 | 0.004319498 | 79072/8531/23367/ |
| OF_CELLULAR_AMIDE_ | | | | | 26986/23185/3646/ |
| METABOLIC_PROCESS | | | | | 8663/8664/2475/84300/ |
| | | | | | 55245/63935 |
| GO_SNRNA_PROCESSING | 6/356 | 36/18046 | 6.67E−05 | 0.004422116 | 25896/11340/56915/ |
| | | | | | 51010/203522/26512 |
| GO_REGULATION_OF_ | 12/356 | 157/18046 | 6.78E−05 | 0.004422116 | 6774/5663/23225/3416/ |
| GENERATION_OF_ | | | | | 55829/4928/8480/ |
| PRECURSOR_METABOLITES_ | | | | | 8021/2475/84300/ |
| AND_ENERGY | | | | | 405/53371 |
| GO_TRANSITION_METAL_ | 6/356 | 37/18046 | 7.84E−05 | 0.005016227 | 1317/540/27032/25800/ |
| ION_TRANSMEMBRANE_ | | | | | 23516/57181 |
| TRANSPORTER_ACTIVITY | | | | | |
| GO_PHOSPHATIDYLCHOLINE_ | 6/356 | 38/18046 | 9.15E−05 | 0.005649461 | 137964/56994/1459/ |
| BIOSYNTHETIC_PROCESS | | | | | 1460/1457/2181 |
| GO_SMALL_SUBUNIT_ | 6/356 | 38/18046 | 9.15E−05 | 0.005649461 | 55127/9136/10199/ |
| PROCESSOME | | | | | 79954/25983/27340 |
| GO_REGULATION_OF_CELL_ | 14/356 | 214/18046 | 9.39E−05 | 0.005694299 | 1781/80184/9738/5116/ |
| CYCLE_G2_M_PHASE_ | | | | | 11116/5566/5108/ |
| TRANSITION | | | | | 9662/55755/10142/ |
| | | | | | 11190/22994/22981/ |
| | | | | | 5704 |
| GO_CELL_CYCLE_G2_M_ | 16/356 | 271/18046 | 0.000102072 | 0.006025499 | 1781/80184/9738/4660/ |
| PHASE_TRANSITION | | | | | 5116/11116/5566/ |
| | | | | | 5108/9662/55755/ |
| | | | | | 10142/11190/22994/ |
| | | | | | 22981/5704/54850 |
| GO_ACTIN_FILAMENT_ | 8/356 | 74/18046 | 0.000102836 | 0.006025499 | 3983/7168/7171/79784/ |
| BUNDLE | | | | | 22998/4627/9531/ |
| | | | | | 2275 |
| GO_RIBOSOME_BINDING | 7/356 | 57/18046 | 0.000124134 | 0.007152189 | 90850/27335/25875/ |
| | | | | | 6731/6728/2475/10985 |
| GO_TOR_COMPLEX | 4/356 | 14/18046 | 0.000127463 | 0.007155666 | 9675/9894/23367/2475 |
| GO_ACTIN_BINDING | 21/356 | 428/18046 | 0.000128334 | 0.007155666 | 55219/3983/10755/ |
| | | | | | 2934/7168/7111/5962/ |
| | | | | | 7171/4643/2314/79784/ |
| | | | | | 399687/4645/4646/ |
| | | | | | 22998/4644/4627/ |
| | | | | | 10296/2275/4649/9463 |
| GO_RRNA_METABOLIC_ | 14/356 | 221/18046 | 0.000132032 | 0.007245019 | 55127/9136/26156/ |
| PROCESS | | | | | 10199/1662/9790/57647/ |
| | | | | | 11340/79954/25983/ |
| | | | | | 56915/51010/27340/ |
| | | | | | 23195 |
| GO_CELL_REDOX_ | 8/356 | 77/18046 | 0.000136406 | 0.0072547 | 2876/169714/55829/ |
| HOMEOSTASIS | | | | | 80142/5034/30001/ |
| | | | | | 23071/9601 |
| GO_PRERIBOSOME | 8/356 | 77/18046 | 0.000136406 | 0.0072547 | 55127/9136/26156/ |
| | | | | | 10199/9790/79954/ |
| | | | | | 25983/27340 |
| GO_REGULATION_OF_ | 8/356 | 79/18046 | 0.000163447 | 0.008338772 | 23367/8667/3646/ |
| TRANSLATIONAL_INITIATION | | | | | 27335/8662/1967/ |
| | | | | | 2475/4528 |
| GO_REPLISOME | 5/356 | 27/18046 | 0.000164026 | 0.008338772 | 23649/5422/5557/5558/ |
| | | | | | 5424 |
| GO_TELOMERE_ | 5/356 | 27/18046 | 0.000164026 | 0.008338772 | 23649/5422/5557/5558/ |
| MAINTENANCE_VIA_ | | | | | 5424 |
| SEMI_CONSERVATIVE_ | | | | | |
| REPLICATION | | | | | |
| GO_NUCLEAR_ENVELOPE | 22/356 | 472/18046 | 0.000185158 | 0.009276703 | 10526/5663/55219/ |
| | | | | | 64328/5422/1070/2627/ |
| | | | | | 5108/4646/4008/ |
| | | | | | 23225/10280/169714/ |
| | | | | | 64215/4928/8480/ |
| | | | | | 8021/811/9587/54884/ |
| | | | | | 53371/84514 |
| GO_POSITIVE_REGULATION_ | 11/356 | 153/18046 | 0.000232256 | 0.011470111 | 5663/64328/1459/ |
| OF_INTRACELLULAR_ | | | | | 80184/5116/5566/5108/ |
| PROTEIN_TRANSPORT | | | | | 22994/26229/9531/ |
| | | | | | 5494 |
| GO_ENDOPLASMIC_ | 13/356 | 207/18046 | 0.000248095 | 0.012079767 | 10945/1781/90522/ |
| RETICULUM_TO_GOLGI_ | | | | | 26958/57222/2801/ |
| VESICLE_MEDIATED_ | | | | | 2804/64689/10960/ |
| TRANSPORT | | | | | 126003/10342/4905/ |
| | | | | | 22820 |
| GO_REGULATION_OF_MRNA_ | 17/356 | 325/18046 | 0.000266984 | 0.012818957 | 79072/79675/8531/ |
| METABOLIC_PROCESS | | | | | 8761/23367/8106/ |
| | | | | | 26986/4343/57690/ |
| | | | | | 26058/11340/56915/ |
| | | | | | 51010/4928/8021/2475/ |
| | | | | | 5704 |
| GO_MATURATION_OF_SSU_ | 6/356 | 47/18046 | 0.00030663 | 0.01448285 | 55127/9790/57647/ |
| RRNA | | | | | 79954/25983/27340 |
| GO_MICROTUBULE_ | 10/356 | 133/18046 | 0.000310018 | 0.01448285 | 1070/9738/2801/5108/ |
| ORGANIZING_CENTER_ | | | | | 9662/55755/11190/ |
| ORGANIZATION | | | | | 22994/51199/26973 |
| GO_REGULATION_OF_ | 8/356 | 87/18046 | 0.000319423 | 0.014723257 | 6774/5663/23225/4928/ |
| CARBOHYDRATE_ | | | | | 8480/8021/405/53371 |
| CATABOLIC_PROCESS | | | | | |
| GO_NCRNA_3_END_ | 6/356 | 48/18046 | 0.000344685 | 0.015678618 | 25896/11340/56915/ |
| PROCESSING | | | | | 51010/203522/26512 |
| GO_MOTOR_ACTIVITY | 10/356 | 136/18046 | 0.000370764 | 0.016141843 | 1781/10513/140465/ |
| | | | | | 4643/79784/4645/4646/ |
| | | | | | 4644/4627/4649 |
| GO_90S_PRERIBOSOME | 5/356 | 32/18046 | 0.000377372 | 0.016141843 | 55127/26156/10199/ |
| | | | | | 9790/27340 |
| GO_PROTEIN_LOCALIZATION_ | 5/356 | 32/18046 | 0.000377372 | 0.016141843 | 2804/5108/10464/11190/ |
| TO_MICROTUBULE_ | | | | | 22994 |
| ORGANIZING_CENTER | | | | | |
| GO_TRANSLATION_ | 5/356 | 32/18046 | 0.000377372 | 0.016141843 | 23367/8665/10480/8663/ |
| INITIATION_FACTOR_BINDING | | | | | 8662 |
| GO_RIBOSOMAL_SMALL_ | 7/356 | 68/18046 | 0.000378215 | 0.016141843 | 55127/6838/9790/57647/ |
| SUBUNIT_BIOGENESIS | | | | | 79954/25983/27340 |
| GO_INTRAMOLECULAR_ | 6/356 | 49/18046 | 0.000386339 | 0.016287476 | 169714/80142/5034/ |
| OXIDOREDUCTASE_ACTIVITY | | | | | 30001/23071/9601 |
| GO_NCRNA_METABOLIC_ | 21/356 | 471/18046 | 0.000465221 | 0.019376745 | 55127/25896/9136/ |
| PROCESS | | | | | 26156/55621/10199/ |
| | | | | | 1662/9790/56257/ |
| | | | | | 57647/11340/79954/ |
| | | | | | 25983/56915/51010/ |
| | | | | | 27340/27044/203522/ |
| | | | | | 55699/23195/26512 |
| GO_VIRAL_GENE_ | 12/356 | 194/18046 | 0.000488403 | 0.020100116 | 25873/23225/8665/ |
| EXPRESSION | | | | | 8666/8661/51386/8664/ |
| | | | | | 8662/4928/8480/8021/ |
| | | | | | 53371 |
| GO_ION_TRANSMEMBRANE_ | 5/356 | 34/18046 | 0.000504876 | 0.020533599 | 481/490/493/540/ |
| TRANSPORTER_ACTIVITY_ | | | | | 27032 |
| PHOSPHORYLATIVE_ | | | | | |
| MECHANISM | | | | | |
| GO_NCRNA_PROCESSING | 18/356 | 378/18046 | 0.00054824 | 0.021883989 | 55127/25896/9136/ |
| | | | | | 26156/55621/10199/ |
| | | | | | 1662/9790/57647/ |
| | | | | | 11340/79954/25983/ |
| | | | | | 56915/51010/27340/ |
| | | | | | 203522/23195/26512 |
| GO_ACTIN_FILAMENT_ | 10/356 | 143/18046 | 0.000551868 | 0.021883989 | 7168/140465/7111/ |
| BASED_MOVEMENT | | | | | 7171/4643/79784/ |
| | | | | | 10142/4646/4644/4627 |
| GO_MYOSIN_II_COMPLEX | 4/356 | 20/18046 | 0.000561794 | 0.021883989 | 140465/79784/22998/ |
| | | | | | 4627 |
| GO_PROTEASOMAL_PROTEIN_ | 21/356 | 478/18046 | 0.0005634 | 0.021883989 | 26046/5663/201595/ |
| CATABOLIC_PROCESS | | | | | 267/79699/10755/ |
| | | | | | 5566/8975/8924/10296/ |
| | | | | | 64795/2876/55829/ |
| | | | | | 11101/23392/9373/ |
| | | | | | 56886/5704/9529/10273/ |
| | | | | | 54850 |
| GO_CILIUM_ORGANIZATION | 18/356 | 381/18046 | 0.000601202 | 0.023092824 | 1781/80184/9738/ |
| | | | | | 219844/3983/5116/ |
| | | | | | 11116/2934/5566/5108/ |
| | | | | | 9662/10464/55755/ |
| | | | | | 10142/11190/22994/ |
| | | | | | 22981/4218 |
| GO_NEGATIVE_REGULATION_ | 14/356 | 259/18046 | 0.000662952 | 0.02460442 | 493/6774/5663/8531/ |
| OF_CELLULAR_CATABOLIC_ | | | | | 1459/23367/26986/ |
| PROCESS | | | | | 1457/10755/2801/ |
| | | | | | 26073/51025/2475/ |
| | | | | | 9529 |
| GO_REGULATION_OF_ATP_ | 9/356 | 121/18046 | 0.000664738 | 0.02460442 | 6774/5663/23225/4928/ |
| METABOLIC_PROCESS | | | | | 8480/8021/84300/405/ |
| | | | | | 53371 |
| GO_CALMODULIN_BINDING | 12/356 | 201/18046 | 0.000669433 | 0.02460442 | 490/493/29966/5116/ |
| | | | | | 4643/79784/55755/ |
| | | | | | 4645/4646/4644/4627/ |
| | | | | | 23352 |
| GO_POSITIVE_REGULATION_ | 12/356 | 201/18046 | 0.000669433 | 0.02460442 | 5663/2934/7168/55755/ |
| OF_SUPRAMOLECULAR_ | | | | | 10142/22998/51199/ |
| FIBER_ORGANIZATION | | | | | 382/2876/2475/ |
| | | | | | 79709/9463 |
| GO_GAMMA_TUBULIN_ | 4/356 | 21/18046 | 0.000683258 | 0.02460442 | 10426/10844/80184/ |
| COMPLEX | | | | | 55755 |
| GO_POSITIVE_REGULATION_ | 4/356 | 21/18046 | 0.000683258 | 0.02460442 | 64328/5566/9531/5494 |
| OF_PROTEIN_EXPORT_FROM_ | | | | | |
| NUCLEUS | | | | | |
| GO_MICROTUBULE_ | 7/356 | 76/18046 | 0.0007457 | 0.026576131 | 10426/10844/2801/ |
| POLYMERIZATION | | | | | 55755/10142/51199/ |
| | | | | | 10048 |
| GO_RNA_3_END_PROCESSING | 10/356 | 150/18046 | 0.000800776 | 0.027132745 | 25896/8106/26986/ |
| | | | | | 10569/51692/11340/ |
| | | | | | 56915/51010/203522/ |
| | | | | | 26512 |
| GO_MACROAUTOPHAGY | 15/356 | 295/18046 | 0.000808835 | 0.027132745 | 823/5663/1459/1460/ |
| | | | | | 23367/1457/8897/5566/ |
| | | | | | 26073/2475/9373/ |
| | | | | | 9868/9531/23557/ |
| | | | | | 55626 |
| GO_ACTIN_FILAMENT_ | 3/356 | 10/18046 | 0.000824107 | 0.027132745 | 2934/2314/4627 |
| SEVERING | | | | | |
| GO_CALCIUM_ | 3/356 | 10/18046 | 0.000824107 | 0.027132745 | 490/493/27032 |
| TRANSMEMBRANE_ | | | | | |
| TRANSPORTER_ACTIVITY_ | | | | | |
| PHOSPHORYLATIVE_ | | | | | |
| MECHANISM | | | | | |
| GO_ER_MEMBRANE_ | 3/356 | 10/18046 | 0.000824107 | 0.027132745 | 9694/23065/56851 |
| PROTEIN_COMPLEX | | | | | |
| GO_MICROTUBULE_ | 3/356 | 10/18046 | 0.000824107 | 0.027132745 | 5108/51199/22981 |
| ANCHORING_AT_ | | | | | |
| CENTROSOME | | | | | |
| GO_NUCLEAR_TRANSCRIBED_ | 3/356 | 10/18046 | 0.000824107 | 0.027132745 | 11340/56915/51010 |
| MRNA_CATABOLIC_PROCESS_ | | | | | |
| EXONUCLEOLYTIC_3_5 | | | | | |
| GO_REGULATION_OF_MRNA_ | 3/356 | 10/18046 | 0.000824107 | 0.027132745 | 3646/8663/8664 |
| BINDING | | | | | |
| GO_MRNA_TRANSPORT | 10/356 | 151/18046 | 0.000842884 | 0.027489154 | 8106/9908/1070/10569/ |
| | | | | | 23225/51692/4928/ |
| | | | | | 8480/8021/53371 |
| GO_NCRNA_EXPORT_FROM_ | 5/356 | 38/18046 | 0.000853866 | 0.027587044 | 23225/4928/8480/8021/ |
| NUCLEUS | | | | | 53371 |
| GO_POSITIVE_REGULATION_ | 12/356 | 207/18046 | 0.000866497 | 0.02773594 | 5663/64328/1459/ |
| OF_INTRACELLULAR_ | | | | | 80184/5116/5566/5962/ |
| TRANSPORT | | | | | 5108/22994/26229/ |
| | | | | | 9531/5494 |
| GO_ADP_BINDING | 5/356 | 39/18046 | 0.000963791 | 0.030567201 | 399687/4646/4627/ |
| | | | | | 1727/26973 |
| GO_PROTEIN_SUMOYLATION | 7/356 | 81/18046 | 0.001090727 | 0.034278573 | 54472/23225/4928/ |
| | | | | | 8480/8021/405/53371 |
| GO_TORC2_COMPLEX | 3/356 | 11/18046 | 0.001116632 | 0.034776544 | 9675/9894/2475 |
| GO_MICROBODY_MEMBRANE | 6/356 | 60/18046 | 0.001153579 | 0.035606456 | 8504/5195/3615/2181/ |
| | | | | | 5824/51 |
| GO_RNA_CATABOLIC_ | 18/356 | 404/18046 | 0.001174671 | 0.035936625 | 79072/79675/8531/ |
| PROCESS | | | | | 8761/23367/26986/ |
| | | | | | 4343/25873/57690/ |
| | | | | | 3646/26058/11340/ |
| | | | | | 56915/51010/8021/ |
| | | | | | 2475/5704/27044 |
| GO_PHOSPHATIDYLCHOLINE_ | 7/356 | 83/18046 | 0.001259481 | 0.03819322 | 137964/56994/1459/ |
| METABOLIC_PROCESS | | | | | 1460/1457/949/2181 |
| GO_NUCLEAR_REPLICATION_ | 5/356 | 42/18046 | 0.001356897 | 0.040789508 | 23649/5422/5557/5558/ |
| FORK | | | | | 5424 |
| GO_PROTEIN_N_TERMINUS_ | 8/356 | 109/18046 | 0.001429241 | 0.042593833 | 1459/1457/5195/382/ |
| BINDING | | | | | 3646/5824/11130/51 |
| GO_MICROBODY | 9/356 | 135/18046 | 0.001446999 | 0.042621737 | 8504/219743/5195/ |
| | | | | | 4644/3615/3416/2181/ |
| | | | | | 5824/51 |
| GO_MICROTUBULE_ | 3/356 | 12/18046 | 0.001467164 | 0.042621737 | 5108/51199/22981 |
| ANCHORING_AT_ | | | | | |
| MICROTUBULE_ | | | | | |
| ORGANIZING_CENTER | | | | | |
| GO_REGULATION_OF_RNA_ | 3/356 | 12/18046 | 0.001467164 | 0.042621737 | 3646/8663/8664 |
| BINDING | | | | | |
| GO_NEGATIVE_REGULATION_ | 15/356 | 314/18046 | 0.001508493 | 0.042700284 | 493/6774/5663/8531/ |
| OF_CATABOLIC_PROCESS | | | | | 1459/23367/26986/ |
| | | | | | 1457/64784/10755/ |
| | | | | | 2801/26073/51025/ |
| | | | | | 2475/9529 |
| GO_REGULATION_OF_CELL_ | 20/356 | 482/18046 | 0.0015108 | 0.042700284 | 493/1781/80184/9738/ |
| CYCLE_PHASE_TRANSITION | | | | | 8737/5116/11116/ |
| | | | | | 5566/5962/5108/9662/ |
| | | | | | 55755/10142/11190/ |
| | | | | | 22994/22981/26058/ |
| | | | | | 56257/5704/9587 |
| GO_CELL_SUBSTRATE_ | 18/356 | 414/18046 | 0.001541786 | 0.042700284 | 823/10146/26986/ |
| JUNCTION | | | | | 90102/2934/5576/5962/ |
| | | | | | 7171/2314/4627/4008/ |
| | | | | | 382/51056/26136/ |
| | | | | | 5034/811/2275/2274 |
| GO_PDZ_DOMAIN_BINDING | 7/356 | 86/18046 | 0.001550175 | 0.042700284 | 490/493/5663/10755/ |
| | | | | | 23085/4905/51 |
| GO_RETROGRADE_VESICLE_ | 7/356 | 86/18046 | 0.001550175 | 0.042700284 | 10945/26958/57222/ |
| MEDIATED_TRANSPORT_ | | | | | 10960/4905/22820/ |
| GOLGI_TO_ENDOPLASMIC_ | | | | | 9463 |
| RETICULUM | | | | | |
| GO_REGULATION_OF_ | 13/356 | 252/18046 | 0.001557894 | 0.042700284 | 5663/1459/1457/79699/ |
| CELLULAR_PROTEIN_ | | | | | 10755/5566/5962/ |
| CATABOLIC_PROCESS | | | | | 8975/2876/84300/ |
| | | | | | 5704/9529/10273 |
| GO_REGULATION_OF_ | 17/356 | 381/18046 | 0.001570807 | 0.042700284 | 5663/267/1460/5195/ |
| BINDING | | | | | 5566/2801/382/3646/ |
| | | | | | 8663/8664/56257/ |
| | | | | | 57326/5824/4140/2011/ |
| | | | | | 10273/23557 |
| GO_IMPORT_INTO_NUCLEUS | 10/356 | 164/18046 | 0.001576641 | 0.042700284 | 10526/9670/6774/5663/ |
| | | | | | 4928/8021/30000/ |
| | | | | | 55027/9531/53371 |
| GO_UBIQUITIN_LIGASE_ | 14/356 | 284/18046 | 0.001601861 | 0.042700284 | 267/84231/79699/4008/ |
| COMPLEX | | | | | 51646/57610/10296/ |
| | | | | | 10048/80232/64795/ |
| | | | | | 54994/10238/10273/ |
| | | | | | 54850 |
| GO_MITOCHONDRIAL_ | 9/356 | 137/18046 | 0.001602733 | 0.042700284 | 10240/79072/84545/ |
| TRANSLATION | | | | | 64969/65003/84300/ |
| | | | | | 55245/4528/55699 |
| GO_MRNA_EXPORT_FROM_ | 8/356 | 111/18046 | 0.001605738 | 0.042700284 | 8106/10569/23225/ |
| NUCLEUS | | | | | 51692/4928/8480/8021/ |
| | | | | | 53371 |
| GO_MITOCHONDRIAL_GENE_ | 10/356 | 165/18046 | 0.00164969 | 0.043534193 | 10240/79072/60493/ |
| EXPRESSION | | | | | 84545/64969/65003/ |
| | | | | | 84300/55245/4528/ |
| | | | | | 55699 |
| GO_MITOTIC_SPINDLE_POLE | 4/356 | 27/18046 | 0.001825239 | 0.047776996 | 55755/51199/51646/ |
| | | | | | 8480 |
| GO_TAU_PROTEIN_BINDING | 5/356 | 45/18046 | 0.00185715 | 0.047776996 | 26574/4140/2011/4139/ |
| | | | | | 10273 |
| GO_ALPHA_LINOLENIC_ACID_ | 3/356 | 13/18046 | 0.001879569 | 0.047776996 | 9415/60481/51 |
| METABOLIC_PROCESS | | | | | |
| GO_CENTRIOLE_CENTRIOLE_ | 3/356 | 13/18046 | 0.001879569 | 0.047776996 | 9662/11190/51199 |
| COHESION | | | | | |
| GO_NUCLEAR_INCLUSION_ | 3/356 | 13/18046 | 0.001879569 | 0.047776996 | 8106/4928/10273 |
| BODY | | | | | |
| GO_REGULATION_OF_ | 12/356 | 227/18046 | 0.001903619 | 0.048035119 | 5663/64328/1459/ |
| INTRACELLULAR_PROTEIN_ | | | | | 80184/5116/5566/ |
| TRANSPORT | | | | | 5108/22994/26229/ |
| | | | | | 9531/5494/53371 |

TABLE 9I
SARS-COV-2

| Description | GeneRatio | BgRatio | pvalue | p.adjust | geneID |
|---|---|---|---|---|---|
| GO_PROTEIN_TARGETING | 30/374 | 428/18046 | 6.46E−09 | 2.40E−05 | 8546/9512/2040/23203/ |
| | | | | | 10531/1459/25873/51125/ |
| | | | | | 80273/219743/9648/5189/ |
| | | | | | 252983/11001/3416/26519/ |
| | | | | | 90580/26515/26520/8540/ |
| | | | | | 7879/131118/6731/6728/ |
| | | | | | 6729/53371/26521/55823/ |
| | | | | | 10956/9868 |
| GO_PROTEIN_TARGETING_ | 13/374 | 101/18046 | 1.66E−07 | 0.000309267 | 9512/23203/10531/1459/ |
| TO_MITOCHONDRION | | | | | 80273/26519/90580/26515/ |
| | | | | | 26520/131118/26521/55823/ |
| | | | | | 9868 |
| GO_MITOCHONDRIAL_ | 20/374 | 260/18046 | 5.36E−07 | 0.000664267 | 9512/23203/80273/10295/ |
| PROTEIN_COMPLEX | | | | | 1763/26519/90580/55735/ |
| | | | | | 26515/26520/10632/131118/ |
| | | | | | 51116/64969/23107/26521/ |
| | | | | | 9868/617/51103/4715 |
| GO_NCRNA_EXPORT_FROM_ | 8/374 | 38/18046 | 8.97E−07 | 0.000817847 | 23225/8021/23636/53371/ |
| NUCLEUS | | | | | 4927/9818/4928/8480 |
| GO_STRUCTURAL_ | 7/374 | 28/18046 | 1.26E−06 | 0.000817847 | 10204/8021/23636/53371/ |
| CONSTITUENT_OF_ | | | | | 4927/9818/4928 |
| NUCLEAR_PORE | | | | | |
| GO_ENDOMEMBRANE_ | 26/374 | 436/18046 | 1.53E−06 | 0.000817847 | 196527/57142/26993/11113/ |
| SYSTEM_ORGANIZATION | | | | | 2801/2804/9659/9648/10142/ |
| | | | | | 64689/51361/23325/7879/ |
| | | | | | 5862/10890/5861/10960/ |
| | | | | | 26092/22931/91754/55823/ |
| | | | | | 25777/1861/27243/9529/ |
| | | | | | 50999 |
| GO_CELLULAR_RESPONSE_ | 14/374 | 142/18046 | 1.54E−06 | 0.000817847 | 10569/3281/5566/23225/ |
| TO_HEAT | | | | | 3066/8021/23636/53371/ |
| | | | | | 4927/9818/3162/4928/8480/ |
| | | | | | 9529 |
| GO_RETROGRADE_ | 7/374 | 30/18046 | 2.10E−06 | 0.000973987 | 56850/10311/28952/54520/ |
| TRANSPORT_ENDOSOME_ | | | | | 57020/4218/23339 |
| TO_PLASMA_MEMBRANE | | | | | |
| GO_GDP_BINDING | 10/374 | 74/18046 | 2.86E−06 | 0.000997832 | 5898/5878/7879/4218/5862/ |
| | | | | | 10890/51552/387/22931/ |
| | | | | | 6729 |
| GO_MRNA_TRANSPORT | 14/374 | 151/18046 | 3.20E−06 | 0.000997832 | 26993/5976/9908/10569/ |
| | | | | | 10204/23225/51692/8021/ |
| | | | | | 23636/53371/4927/9818/ |
| | | | | | 4928/8480 |
| GO_MRNA_EXPORT_FROM_ | 12/374 | 111/18046 | 3.29E−06 | 0.000997832 | 26993/5976/10569/23225/ |
| NUCLEUS | | | | | 51692/8021/23636/53371/ |
| | | | | | 4927/9818/4928/8480 |
| GO_SNRNA_METABOLIC_ | 8/374 | 45/18046 | 3.48E−06 | 0.000997832 | 92105/57508/25896/56257/ |
| PROCESS | | | | | 11340/56915/51010/23404 |
| GO_VESICLE_MEDIATED_ | 11/374 | 93/18046 | 3.49E−06 | 0.000997832 | 51125/56850/10311/28952/ |
| TRANSPORT_TO_THE_ | | | | | 54520/57020/150684/2181/ |
| PLASMA_MEMBRANE | | | | | 4218/10890/23339 |
| GO_CELL_CYCLE_G2_M_ | 19/374 | 271/18046 | 4.08E−06 | 0.001067475 | 23476/5714/26993/10270/ |
| PHASE_TRANSITION | | | | | 11113/5116/11116/5566/ |
| | | | | | 5577/1063/9662/11064/ |
| | | | | | 55755/10142/11190/22981/ |
| | | | | | 8481/9978/54850 |
| GO_CILIARY_BASAL_BODY_ | 11/374 | 95/18046 | 4.31E−06 | 0.001067475 | 5116/11116/5566/5577/9662/ |
| PLASMA_MEMBRANE_ | | | | | 11064/55755/10142/11190/ |
| DOCKING | | | | | 22981/8481 |
| GO_MEMBRANE_DOCKING | 15/374 | 179/18046 | 5.04E−06 | 0.001145849 | 5116/11116/5566/5577/9662/ |
| | | | | | 11064/55755/10142/11190/ |
| | | | | | 22981/8481/7879/4218/ |
| | | | | | 10890/55823 |
| GO_REGULATION_OF_ | 10/374 | 79/18046 | 5.24E−06 | 0.001145849 | 3281/23225/8021/23636/ |
| CELLULAR_RESPONSE_ | | | | | 53371/4927/9818/4928/ |
| TO_HEAT | | | | | 8480/9529 |
| GO_ERAD_PATHWAY | 11/374 | 99/18046 | 6.46E−06 | 0.001284765 | 8975/29761/55829/1861/ |
| | | | | | 10956/80020/27248/80267/ |
| | | | | | 55757/7993/7466 |
| GO_ENDOPLASMIC_ | 20/374 | 306/18046 | 6.56E−06 | 0.001284765 | 79709/11001/2200/1861/ |
| RETICULUM_LUMEN | | | | | 8614/1291/4240/10956/ |
| | | | | | 79070/143888/80020/27248/ |
| | | | | | 23071/80267/64374/55757/ |
| | | | | | 10525/51661/60681/7466 |
| GO_CENTRIOLE | 13/374 | 141/18046 | 7.64E−06 | 0.001304812 | 10426/5116/11116/9857/ |
| | | | | | 9662/51199/11190/8481/ |
| | | | | | 8924/55165/145508/49856/ |
| | | | | | 4218 |
| GO_PROTEIN_ | 13/374 | 141/18046 | 7.64E−06 | 0.001304812 | 9512/23203/10531/1459/ |
| LOCALIZATION_TO_ | | | | | 80273/26519/90580/26515/ |
| MITOCHONDRION | | | | | 26520/131118/26521/55823/ |
| | | | | | 9868 |
| GO_SNRNA_PROCESSING | 7/374 | 36/18046 | 7.72E−06 | 0.001304812 | 92105/57508/25896/11340/ |
| | | | | | 56915/51010/23404 |
| GO_GOLGI_ORGANIZATION | 13/374 | 142/18046 | 8.26E−06 | 0.001335199 | 11113/2801/2804/9659/ |
| | | | | | 9648/10142/64689/51361/ |
| | | | | | 5862/5861/10960/9529/ |
| | | | | | 50999 |
| GO_CUL2_RING_UBIQUITIN_ | 5/374 | 15/18046 | 9.42E−06 | 0.001459813 | 150684/8453/79699/9978/ |
| LIGASE_COMPLEX | | | | | 6923 |
| GO_CELL_DIVISION_SITE | 9/374 | 70/18046 | 1.37E−05 | 0.002032541 | 10426/10844/11113/5962/ |
| | | | | | 382/55165/5898/387/3688 |
| GO_PROTEIN_FOLDING | 16/374 | 220/18046 | 1.49E−05 | 0.002134264 | 10283/1459/1460/80273/ |
| | | | | | 6902/53938/2782/7841/ |
| | | | | | 131118/1861/56605/55768/ |
| | | | | | 23071/64374/9529/7466 |
| GO_TELOMERE_ | 6/374 | 27/18046 | 1.56E−05 | 0.002146989 | 5976/5422/5557/5558/ |
| MAINTENANCE_VIA_SEMI_ | | | | | 23649/1763 |
| CONSERVATIVE_ | | | | | |
| REPLICATION | | | | | |
| GO_GLYCOPROTEIN_ | 23/374 | 412/18046 | 1.79E−05 | 0.002382707 | 2801/64689/440138/5861/ |
| METABOLIC_PROCESS | | | | | 7841/9653/26574/29880/ |
| | | | | | 5046/10956/79070/143888/ |
| | | | | | 79586/55768/90161/6388/ |
| | | | | | 23071/80267/23509/55757/ |
| | | | | | 54480/23333/79053 |
| GO_ENDOSOMAL_ | 16/374 | 228/18046 | 2.32E−05 | 0.002937493 | 8546/56850/23085/9648/ |
| TRANSPORT | | | | | 382/10311/28952/54520/ |
| | | | | | 57020/23325/7879/4218/ |
| | | | | | 10890/51552/23339/27243 |
| GO_HOST_CELLULAR_ | 9/374 | 75/18046 | 2.41E−05 | 0.002937493 | 4343/23225/8021/23636/ |
| COMPONENT | | | | | 53371/4927/9818/4928/8480 |
| GO_RNA_LOCALIZATION | 16/374 | 229/18046 | 2.45E−05 | 0.002937493 | 26993/5976/9908/10569/ |
| | | | | | 10204/23225/51692/51010/ |
| | | | | | 23404/8021/23636/53371/ |
| | | | | | 4927/9818/4928/8480 |
| GO_ | 5/374 | 18/18046 | 2.55E−05 | 0.002967549 | 29880/79070/143888/55757/ |
| GLUCOSYLTRANSFERASE_ | | | | | 79053 |
| ACTIVITY | | | | | |
| GO_RNA_EXPORT_FROM_ | 12/374 | 136/18046 | 2.66E−05 | 0.002995921 | 26993/5976/10569/23225/ |
| NUCLEUS | | | | | 51692/8021/23636/53371/ |
| | | | | | 4927/9818/4928/8480 |
| GO_RESPONSE_TO_HEAT | 14/374 | 183/18046 | 2.91E−05 | 0.00315232 | 10569/3281/5566/23225/ |
| | | | | | 3066/8021/23636/53371/ |
| | | | | | 4927/9818/3162/4928/8480/ |
| | | | | | 9529 |
| GO_SNRNA_3_END_ | 6/374 | 30/18046 | 2.97E−05 | 0.00315232 | 57508/25896/11340/56915/ |
| PROCESSING | | | | | 51010/23404 |
| GO_NUCLEAR_TRANSCRIBED_ | 4/374 | 10/18046 | 3.45E−05 | 0.003568014 | 11340/56915/51010/23404 |
| MRNA_CATABOLIC_PROCESS_ | | | | | |
| EXONUCLEOLYTIC_3_5 | | | | | |
| GO_MULTI_ORGANISM_ | 8/374 | 62/18046 | 4.02E−05 | 0.004041208 | 23225/8021/23636/53371/ |
| LOCALIZATION | | | | | 4927/9818/4928/8480 |
| GO_REGULATION_OF_CELL_ | 15/374 | 214/18046 | 4.22E−05 | 0.00412939 | 23476/5714/5116/11116/ |
| CYCLE_G2_M_PHASE_ | | | | | 5566/5577/1063/9662/11064/ |
| TRANSITION | | | | | 55755/10142/11190/22981/ |
| | | | | | 8481/9978 |
| GO_CYTOPLASMIC_STRESS_ | 8/374 | 63/18046 | 4.52E−05 | 0.004313284 | 26986/10146/8761/23367/ |
| GRANULE | | | | | 4343/9908/23185/26058 |
| GO_CHAPERONE_MEDIATED_ | 4/374 | 11/18046 | 5.34E−05 | 0.004842662 | 26519/26520/26521/1861 |
| PROTEIN_TRANSPORT | | | | | |
| GO_UDP_ | 4/374 | 11/18046 | 5.34E−05 | 0.004842662 | 29880/79070/143888/55757 |
| GLUCOSYLTRANSFERASE_ | | | | | |
| ACTIVITY | | | | | |
| GO_ENDOPLASMIC_ | 5/374 | 21/18046 | 5.76E−05 | 0.00489194 | 7905/57142/10193/10890/ |
| RETICULUM_TUBULAR_ | | | | | 22931 |
| NETWORK | | | | | |
| GO_NUCLEAR_EXPORT | 14/374 | 195/18046 | 5.84E−05 | 0.00489194 | 26993/5976/10569/5566/ |
| | | | | | 10204/23225/51692/8021/ |
| | | | | | 23636/53371/4927/9818/ |
| | | | | | 4928/8480 |
| GO_VIRAL_LIFE_CYCLE | 19/374 | 328/18046 | 5.88E−05 | 0.00489194 | 2040/26986/23367/22954/ |
| | | | | | 23225/3416/7879/5861/949/ |
| | | | | | 8021/23636/53371/4927/ |
| | | | | | 9818/4928/8480/3688/5817/ |
| | | | | | 27243 |
| GO_I_KAPPAB_KINASE_NF_ | 17/374 | 273/18046 | 5.92E−05 | 0.00489194 | 23476/57153/79753/9188/ |
| KAPPAB_SIGNALING | | | | | 8737/7088/23085/29110/ |
| | | | | | 28952/22954/387/23636/ |
| | | | | | 3162/286827/2150/79671/ |
| | | | | | 54602 |
| GO_ESTABLISHMENT_OF_ | 14/374 | 196/18046 | 6.17E−05 | 0.004913479 | 26993/5976/9908/10569/ |
| RNA_LOCALIZATION | | | | | 10204/23225/51692/8021/ |
| | | | | | 23636/53371/4927/9818/ |
| | | | | | 4928/8480 |
| GO_PROTEIN_KINASE_A_ | 7/374 | 49/18046 | 6.30E−05 | 0.004913479 | 26993/10270/5576/5566/ |
| BINDING | | | | | 5577/5962/10142 |
| GO_PROTEASOMAL_PROTEIN_ | 24/374 | 478/18046 | 6.47E−05 | 0.004913479 | 5714/5566/8975/10193/ |
| CATABOLIC_PROCESS | | | | | 10612/8924/150684/29761/ |
| | | | | | 2876/55829/11101/8453/ |
| | | | | | 79699/9978/1861/10956/ |
| | | | | | 80020/27248/80267/54850/ |
| | | | | | 55757/9529/7993/7466 |
| GO_REGULATION_OF_ | 21/374 | 388/18046 | 6.47E−05 | 0.004913479 | 1459/23077/5566/5962/ |
| PROTEIN_CATABOLIC_ | | | | | 8975/10193/28952/7337/ |
| PROCESS | | | | | 22954/150684/29761/3416/ |
| | | | | | 2876/7879/79699/9978/55823/ |
| | | | | | 10956/27248/8754/9529 |
| GO_DNA_POLYMERASE_ | 5/374 | 22/18046 | 7.33E−05 | 0.005451949 | 5422/5557/5558/23649/1763 |
| COMPLEX | | | | | |
| GO_ENDOPLASMIC_ | 11/374 | 129/18046 | 7.84E−05 | 0.005715577 | 10897/57222/2801/2804/ |
| RETICULUM_GOLGI_ | | | | | 64689/537/5862/10960/ |
| INTERMEDIATE_ | | | | | 23071/55757/50999 |
| COMPARTMENT | | | | | |
| GO_GOLGI_VESICLE_ | 20/374 | 367/18046 | 8.78E−05 | 0.006279921 | 10897/51125/57222/2802/ |
| TRANSPORT | | | | | 2801/2804/9648/64689/ |
| | | | | | 28952/54520/57020/150684/ |
| | | | | | 2181/4218/10890/51552/ |
| | | | | | 5861/10960/10525/50999 |
| GO_CENTRIOLE_CENTRIOLE_ | 4/374 | 13/18046 | 0.000111928 | 0.007731467 | 9662/23177/51199/11190 |
| COHESION | | | | | |
| GO_MIDBODY | 13/374 | 182/18046 | 0.000112363 | 0.007731467 | 11113/5962/1063/11064/ |
| | | | | | 382/51056/55165/5898/4218/ |
| | | | | | 387/51097/23636/23111 |
| GO_REGULATION_OF_BONE_ | 5/374 | 24/18046 | 0.00011434 | 0.007731467 | 5447/537/2200/4015/202018 |
| DEVELOPMENT | | | | | |
| GO_NUCLEAR_PORE | 9/374 | 92/18046 | 0.000122379 | 0.008127307 | 10204/23225/8021/23636/ |
| | | | | | 53371/4927/9818/4928/8480 |
| GO_CLEAVAGE_FURROW | 7/374 | 55/18046 | 0.000133834 | 0.008732111 | 11113/5962/382/55165/5898/ |
| | | | | | 387/3688 |
| GO_ENDOPLASMIC_ | 5/374 | 25/18046 | 0.000140511 | 0.009009631 | 7905/57142/10193/10890/ |
| RETICULUM_ | | | | | 22931 |
| SUBCOMPARTMENT | | | | | |
| GO_NUCLEOBASE_ | 15/374 | 240/18046 | 0.000153305 | 0.009554297 | 26993/5976/9908/10569/ |
| CONTAINING_COMPOUND_ | | | | | 8737/10204/23225/51692/ |
| TRANSPORT | | | | | 8021/23636/53371/4927/ |
| | | | | | 9818/4928/8480 |
| GO_CYTOPLASMIC_ | 4/374 | 14/18046 | 0.000154143 | 0.009554297 | 11340/56915/51010/23404 |
| EXOSOME_RNASE_COMPLEX | | | | | |
| GO_RAB_PROTEIN_SIGNAL_ | 8/374 | 75/18046 | 0.000158809 | 0.009682153 | 5878/7879/4218/5862/10890/ |
| TRANSDUCTION | | | | | 51552/5861/22931 |
| GO_MICROTUBULE_ | 5/374 | 26/18046 | 0.000171028 | 0.010096073 | 11116/9857/9648/51199/ |
| ANCHORING | | | | | 22981 |
| GO_MICROTUBULE_ | 5/374 | 26/18046 | 0.000171028 | 0.010096073 | 10426/10844/2801/51199/ |
| NUCLEATION | | | | | 10142 |
| GO_RNA_SURVEILLANCE | 4/374 | 15/18046 | 0.000206769 | 0.011991982 | 11340/56915/51010/23404 |
| GO_GOLGI_TO_PLASMA_ | 7/374 | 59/18046 | 0.000209594 | 0.011991982 | 51125/28952/54520/57020/ |
| MEMBRANE_TRANSPORT | | | | | 150684/2181/10890 |
| GO_FLAVIN_ADENINE_ | 8/374 | 79/18046 | 0.000228571 | 0.012879609 | 34/2108/2671/5447/8540/ |
| DINUCLEOTIDE_BINDING | | | | | 1727/80020/28976 |
| GO_REGULATION_OF_ | 15/374 | 252/18046 | 0.00026042 | 0.014455232 | 1459/5566/5962/8975/28952/ |
| CELLULAR_PROTEIN_ | | | | | 7337/150684/29761/2876/ |
| CATABOLIC_PROCESS | | | | | 79699/9978/55823/10956/ |
| | | | | | 27248/9529 |
| GO_NUCLEAR_EXOSOME_ | 4/374 | 16/18046 | 0.000271202 | 0.014653625 | 11340/56915/51010/23404 |
| RNASE_COMPLEX | | | | | |
| GO_PROTEIN_SUMOYLATION | 8/374 | 81/18046 | 0.000271874 | 0.014653625 | 23225/8021/23636/53371/ |
| | | | | | 4927/9818/4928/8480 |
| GO_NEGATIVE_REGULATION_ | 6/374 | 44/18046 | 0.000276234 | 0.014675895 | 29761/55829/10956/27248/ |
| OF_RESPONSE_TO_ | | | | | 10525/7466 |
| ENDOPLASMIC_ | | | | | |
| RETICULUM_STRESS | | | | | |
| GO_REGULATION_OF_ | 14/374 | 227/18046 | 0.000289442 | 0.014959797 | 2040/26993/1459/5116/5566/ |
| INTRACELLULAR_PROTEIN_ | | | | | 56850/9648/10204/23636/ |
| TRANSPORT | | | | | 53371/9818/55823/10956/ |
| | | | | | 27248 |
| GO_GLYCOPROTEIN_ | 18/374 | 341/18046 | 0.000289622 | 0.014959797 | 2801/64689/440138/7841/ |
| BIOSYNTHETIC_PROCESS | | | | | 9653/26574/29880/79070/ |
| | | | | | 143888/79586/90161/6388/ |
| | | | | | 80267/23509/55757/54480/ |
| | | | | | 23333/79053 |
| GO_ENDOCYTIC_RECYCLING | 6/374 | 45/18046 | 0.000313232 | 0.015877204 | 382/10311/28952/54520/ |
| | | | | | 57020/51552 |
| GO_UNFOLDED_PROTEIN_ | 10/374 | 127/18046 | 0.000315922 | 0.015877204 | 80273/55027/23195/1861/ |
| BINDING | | | | | 56605/27248/64374/55757/ |
| | | | | | 22937/51103 |
| GO_PROTEIN_CONTAINING_ | 16/374 | 286/18046 | 0.000329364 | 0.016332062 | 26993/5976/10569/56850/ |
| COMPLEX_LOCALIZATION | | | | | 201134/23225/51692/117178/ |
| | | | | | 4218/8021/23636/53371/ |
| | | | | | 4927/9818/4928/8480 |
| GO_MITOCHONDRIAL_ | 15/374 | 258/18046 | 0.000334811 | 0.016383706 | 9512/23203/10531/1459/ |
| TRANSPORT | | | | | 80273/26519/90580/26515/ |
| | | | | | 26520/10632/131118/26521/ |
| | | | | | 55823/30968/9868 |
| GO_CYTOPLASMIC_ | 4/374 | 17/18046 | 0.000348877 | 0.016850308 | 8453/9978/6923/10956 |
| UBIQUITIN_LIGASE_COMPLEX | | | | | |
| GO_NUCLEAR_ENVELOPE | 22/374 | 472/18046 | 0.000367355 | 0.017400219 | 57142/5422/1063/10204/ |
| | | | | | 23225/57508/10280/26092/ |
| | | | | | 169714/8021/23636/53371/ |
| | | | | | 4927/9818/151188/25777/ |
| | | | | | 4928/8480/1861/27243/ |
| | | | | | 23333/27346 |
| GO_REGULATION_OF_INTRA | 18/374 | 348/18046 | 0.00036962 | 0.017400219 | 2040/92840/26993/1459/ |
| CELLULAR_TRANSPORT | | | | | 5116/5566/5962/56850/9648/ |
| | | | | | 10204/8021/23636/53371/ |
| | | | | | 9818/3162/55823/10956/ |
| | | | | | 27248 |
| GO_FLEMMING_BODY | 5/374 | 31/18046 | 0.00040576 | 0.018862786 | 11064/382/55165/5898/ |
| | | | | | 23636 |
| GO_REGULATION_OF_ | 11/374 | 157/18046 | 0.000440685 | 0.019853743 | 23225/3416/387/55829/8021/ |
| GENERATION_OF_ | | | | | 23636/53371/4927/9818/ |
| PRECURSOR_ | | | | | 4928/8480 |
| METABOLITES_AND_ENERGY | | | | | |
| GO_REGULATION_OF_ | 8/374 | 87/18046 | 0.000443595 | 0.019853743 | 23225/8021/23636/53371/ |
| CARBOHYDRATE_ | | | | | 4927/9818/4928/8480 |
| CATABOLIC_PROCESS | | | | | |
| GO_NCRNA_3_END_ | 6/374 | 48/18046 | 0.000447931 | 0.019853743 | 57508/25896/11340/56915/ |
| PROCESSING | | | | | 51010/23404 |
| GO_RAS_PROTEIN_SIGNAL_ | 21/374 | 447/18046 | 0.000448431 | 0.019853743 | 10146/9908/5962/382/25959/ |
| TRANSDUCTION | | | | | 117178/5898/5878/7879/ |
| | | | | | 4218/5862/10890/51552/ |
| | | | | | 387/5861/2782/22931/23636/ |
| | | | | | 3688/1786/2150 |
| GO_MICROTUBULE_ | 10/374 | 133/18046 | 0.000456974 | 0.019993963 | 2801/9662/23177/9648/ |
| ORGANIZING_CENTER_ | | | | | 51199/55755/11190/117178/ |
| ORGANIZATION | | | | | 23636/27243 |
| GO_POSITIVE_REGULATION_ | 12/374 | 184/18046 | 0.000470866 | 0.020362233 | 23476/57153/9188/8737/ |
| OF_I_KAPPAB_KINASE_NF_ | | | | | 29110/28952/22954/387/ |
| KAPPAB_SIGNALING | | | | | 23636/3162/2150/54602 |
| GO_ORGANELLE_ENVELOPE_ | 8/374 | 88/18046 | 0.0004793 | 0.020379019 | 2671/23408/26519/90580/ |
| LUMEN | | | | | 26515/26520/26521/30968 |
| GO_MICROTUBULE_ | 10/374 | 134/18046 | 0.000484918 | 0.020379019 | 5116/2801/55755/49856/ |
| CYTOSKELETON_ | | | | | 387/23636/25777/8480/ |
| ORGANIZATION_ | | | | | 3688/27243 |
| INVOLVED_IN_MITOSIS | | | | | |
| GO_REGULATION_OF_CELL_ | 22/374 | 482/18046 | 0.000487694 | 0.020379019 | 23476/5714/8737/5116/ |
| CYCLE_PHASE_TRANSITION | | | | | 11116/5566/5577/5962/1063/ |
| | | | | | 9662/11064/55755/10142/ |
| | | | | | 11190/22981/8481/25959/ |
| | | | | | 252983/26058/56257/9978/ |
| | | | | | 9510 |
| GO_NEGATIVE_REGULATION_ | 9/374 | 111/18046 | 0.000504692 | 0.020854991 | 57142/8737/10505/6789/ |
| OF_DEVELOPMENTAL_ | | | | | 6788/60485/23111/8614/ |
| GROWTH | | | | | 9518 |
| GO_INNER_MITOCHONDRIAL_ | 10/374 | 135/18046 | 0.000514265 | 0.021017057 | 80273/26519/90580/55735/ |
| MEMBRANE_PROTEIN_ | | | | | 26515/10632/131118/617/ |
| COMPLEX | | | | | 51103/4715 |
| GO_RRNA_CATABOLIC_ | 4/374 | 19/18046 | 0.000549846 | 0.022164753 | 11340/56915/51010/23404 |
| PROCESS | | | | | |
| GO_CADHERIN_BINDING | 17/374 | 330/18046 | 0.000559172 | 0.022164753 | 57142/28969/23367/5318/ |
| | | | | | 55833/5962/2802/2801/ |
| | | | | | 23085/90102/8496/26058/ |
| | | | | | 10890/5861/7458/3688/2011 |
| GO_NEGATIVE_REGULATION_ | 6/374 | 50/18046 | 0.000560228 | 0.022164753 | 8737/7088/28952/387/ |
| OF_I_KAPPAB_KINASE_NF_ | | | | | 286827/79671 |
| KAPPAB_SIGNALING | | | | | |
| GO_NUCLEAR_ENVELOPE_ | 6/374 | 51/18046 | 0.000623995 | 0.024427762 | 26993/26092/91754/25777/ |
| ORGANIZATION | | | | | 1861/27243 |
| GO_MYOSIN_BINDING | 7/374 | 71/18046 | 0.000660835 | 0.02560048 | 22954/5898/4218/10890/ |
| | | | | | 51552/387/9368 |
| GO_PORE_COMPLEX_ | 4/374 | 20/18046 | 0.000676144 | 0.025658981 | 196527/57142/51248/4928 |
| ASSEMBLY | | | | | |
| GO_PROTEIN_KINASE_A_ | 4/374 | 20/18046 | 0.000676144 | 0.025658981 | 26993/10270/5566/10142 |
| REGULATORY_SUBUNIT_ | | | | | |
| BINDING | | | | | |
| GO_REGULATION_OF_ | 9/374 | 116/18046 | 0.000695716 | 0.026135014 | 8737/23225/8021/23636/ |
| POSTTRANSCRIPTIONAL_ | | | | | 53371/4927/9818/4928/8480 |
| GENE_SILENCING | | | | | |
| GO_ENDOPLASMIC_ | 7/374 | 72/18046 | 0.000719197 | 0.026746948 | 57222/2801/64689/537/ |
| RETICULUM_GOLGI_ | | | | | 5862/10960/50999 |
| INTERMEDIATE_ | | | | | |
| COMPARTMENT_ | | | | | |
| MEMBRANE | | | | | |
| GO_RESPONSE_TO_ | 14/374 | 249/18046 | 0.000728973 | 0.026842079 | 10569/3281/5566/23225/ |
| TEMPERATURE_STIMULUS | | | | | 3066/8021/23636/53371/ |
| | | | | | 4927/9818/3162/4928/8480/ |
| | | | | | 9529 |
| GO_UBIQUITIN_LIKE_ | 16/374 | 309/18046 | 0.000763632 | 0.027842614 | 57142/8737/5576/5566/5577/ |
| PROTEIN_LIGASE_BINDING | | | | | 8975/8924/29761/9470/ |
| | | | | | 5898/23111/8453/9978/ |
| | | | | | 6923/9529/7466 |
| GO_POSITIVE_REGULATION_ | 7/374 | 74/18046 | 0.00084813 | 0.030623273 | 10146/9908/9662/49856/387/ |
| OF_ORGANELLE_ASSEMBLY | | | | | 23636/202018 |
| GO_REGULATION_OF_MRNA_ | 12/374 | 199/18046 | 0.000941327 | 0.033050086 | 5714/26986/8761/23367/ |
| CATABOLIC_PROCESS | | | | | 5976/4343/26058/11340/ |
| | | | | | 56915/51010/23404/8021 |
| GO_REGULATION_OF_ATP_ | 9/374 | 121/18046 | 0.000942039 | 0.033050086 | 23225/387/8021/23636/ |
| METABOLIC_PROCESS | | | | | 53371/4927/9818/4928/8480 |
| GO_CAMP_DEPENDENT_ | 3/374 | 10/18046 | 0.00095089 | 0.033050086 | 5576/5566/5577 |
| PROTEIN_KINASE_COMPLEX | | | | | |
| GO_EXTRACELLULAR_ | 3/374 | 10/18046 | 0.00095089 | 0.033050086 | 2200/2201/10516 |
| MATRIX_CONSTITUENT_ | | | | | |
| CONFERRING_ELASTICITY | | | | | |
| GO_TAU_PROTEIN_KINASE_ | 4/374 | 22/18046 | 0.000987988 | 0.034021538 | 23387/4140/2011/4139 |
| ACTIVITY | | | | | |
| GO_RESPONSE_TO_ | 15/374 | 288/18046 | 0.001042722 | 0.035576919 | 10897/8975/29761/55829/ |
| ENDOPLASMIC_RETICULUM_ | | | | | 1861/8614/10956/80020/ |
| STRESS | | | | | 27248/23071/80267/55757/ |
| | | | | | 10525/7993/7466 |
| GO_RIBOSOME_BIOGENESIS | 15/374 | 290/18046 | 0.001117506 | 0.03778186 | 9136/9188/10199/1662/ |
| | | | | | 25983/11340/79954/56915/ |
| | | | | | 51010/26574/51116/23404/ |
| | | | | | 4927/55027/23195 |
| GO_UBIQUITIN_DEPENDENT_ | 7/374 | 78/18046 | 0.001160367 | 0.0387237 | 55829/10956/80020/27248/ |
| ERAD_PATHWAY | | | | | 80267/7993/7466 |
| GO_ENDOPLASMIC_ | 4/374 | 23/18046 | 0.001176601 | 0.0387237 | 10956/27248/80267/55757 |
| RETICULUM_QUALITY_ | | | | | |
| CONTROL_COMPARTMENT | | | | | |
| GO_MITOTIC_CYTOKINETIC_ | 4/374 | 23/18046 | 0.001176601 | 0.0387237 | 55165/387/23636/27243 |
| PROCESS | | | | | |
| GO_POST_GOLGI_VESICLE_ | 8/374 | 101/18046 | 0.001194702 | 0.038974528 | 51125/28952/54520/57020/ |
| MEDIATED_TRANSPORT | | | | | 150684/2181/10890/51552 |
| GO_PROTEIN_INSERTION_ | 3/374 | 11/18046 | 0.001287453 | 0.041635113 | 26519/90580/26520 |
| INTO_MITOCHONDRIAL_ | | | | | |
| INNER_MEMBRANE | | | | | |
| GO_ATPASE_BINDING | 7/374 | 80/18046 | 0.001346993 | 0.042832181 | 481/5962/29761/5898/26092/ |
| | | | | | 55829/7466 |
| GO_ATPASE_REGULATOR_ | 5/374 | 40/18046 | 0.001349121 | 0.042832181 | 481/80273/26092/131118/ |
| ACTIVITY | | | | | 64374 |
| GO_ESTABLISHMENT_OF_ | 6/374 | 59/18046 | 0.001359021 | 0.042832181 | 51125/56850/64689/2181/ |
| PROTEIN_LOCALIZATION_TO_ | | | | | 4218/10890 |
| PLASMA_MEMBRANE | | | | | |
| GO_RESPONSE_TO_OXYGEN_ | 18/374 | 391/18046 | 0.001414735 | 0.043960913 | 481/523/5714/3066/537/387/ |
| LEVELS | | | | | 2782/26355/8453/9978/ |
| | | | | | 6921/6923/3162/5352/8614/ |
| | | | | | 5327/10525/22937 |
| GO_REGULATION_OF_GENE_ | 10/374 | 154/18046 | 0.001418475 | 0.043960913 | 8737/23225/8021/23636/ |
| SILENCING | | | | | 53371/4927/9818/4928/ |
| | | | | | 8480/1786 |
| GO_MICROBODY_MEMBRANE | 6/374 | 60/18046 | 0.001484122 | 0.045241382 | 3615/5189/11001/8540/2181/ |
| | | | | | 55711 |
| GO_NUCLEAR_INNER_ | 6/374 | 60/18046 | 0.001484122 | 0.045241382 | 10204/10280/26092/151188/ |
| MEMBRANE | | | | | 25777/23333 |
| GO_NUCLEAR_MEMBRANE | 15/374 | 299/18046 | 0.001512476 | 0.045730865 | 10204/23225/57508/10280/ |
| | | | | | 26092/169714/23636/53371/ |
| | | | | | 9818/151188/25777/4928/ |
| | | | | | 1861/23333/27346 |
| GO_MAINTENANCE_OF_ | 8/374 | 105/18046 | 0.001534842 | 0.046032881 | 9908/28952/2200/2201/ |
| PROTEIN_LOCATION | | | | | 25777/10956/8733/202018 |
| GO_LIPID_DROPLET | 7/374 | 82/18046 | 0.001556286 | 0.046082687 | 10280/2181/1727/5878/7879/ |
| | | | | | 51097/23111 |
| GO_NUCLEUS_ | 9/374 | 130/18046 | 0.001561285 | 0.046082687 | 57142/26993/26092/53371/ |
| ORGANIZATION | | | | | 91754/25777/4928/1861/ |
| | | | | | 27243 |
| GO_POST_TRANSLATIONAL_ | 17/374 | 363/18046 | 0.001586563 | 0.046460075 | 5714/28952/10489/150684/ |
| PROTEIN_MODIFICATION | | | | | 4218/5862/5861/2200/10238/ |
| | | | | | 8453/9978/6921/6923/ |
| | | | | | 8614/4240/54850/7466 |
| GO_HEPATOCYTE_ | 3/374 | 12/18046 | 0.001690346 | 0.047266145 | 382/6789/6788 |
| APOPTOTIC_PROCESS | | | | | |
| GO_HOPS_COMPLEX | 3/374 | 12/18046 | 0.001690346 | 0.047266145 | 51361/23339/55823 |
| GO_MAINTENANCE_OF_ | 3/374 | 12/18046 | 0.001690346 | 0.047266145 | 10956/8733/202018 |
| PROTEIN_LOCALIZATION_IN_ | | | | | |
| ENDOPLASMIC_RETICULUM | | | | | |
| GO_POSITIVE_REGULATION_ | 3/374 | 12/18046 | 0.001690346 | 0.047266145 | 2801/64689/5861 |
| OF_UBIQUITIN_PROTEIN_ | | | | | |
| LIGASE_ACTIVITY | | | | | |
| GO_SNORNA_3_END_ | 3/374 | 12/18046 | 0.001690346 | 0.047266145 | 56915/51010/23404 |
| PROCESSING | | | | | |
| GO_STRUCTURAL_ | 3/374 | 12/18046 | 0.001690346 | 0.047266145 | 2200/2201/10516 |
| MOLECULE_ACTIVITY_ | | | | | |
| CONFERRING_ELASTICITY | | | | | |
| GO_ATP_METABOLIC_ | 15/374 | 303/18046 | 0.001722322 | 0.04743457 | 481/523/23225/10632/387/ |
| PROCESS | | | | | 8021/23636/53371/4927/ |
| | | | | | 9818/30968/4928/8480/ |
| | | | | | 51103/4715 |
| GO_MITOTIC_SPINDLE_ | 8/374 | 107/18046 | 0.001731505 | 0.04743457 | 5116/2801/49856/387/23636/ |
| ORGANIZATION | | | | | 25777/8480/27243 |
| GO_POSITIVE_REGULATION_ | 19/374 | 431/18046 | 0.001734633 | 0.04743457 | 26986/23367/5976/4343/ |
| OF_CATABOLIC_PROCESS | | | | | 5962/8975/79443/29110/ |
| | | | | | 10193/28952/22954/26058/ |
| | | | | | 3416/7879/79699/9978/ |
| | | | | | 3162/55823/8754 |
| GO_SPLICEOSOMAL_ | 11/374 | 186/18046 | 0.001771706 | 0.048094702 | 10283/25980/26986/79753/ |
| COMPLEX | | | | | 5976/55131/10569/53938/ |
| | | | | | 154007/55599/58155 |
| GO_REGULATION_OF_ | 7/374 | 84/18046 | 0.001790073 | 0.048241159 | 8975/29761/55829/10956/ |
| RESPONSE_TO_ | | | | | 27248/10525/7466 |
| ENDOPLASMIC_RETICULUM_ | | | | | |
| STRESS | | | | | |
| GO_TRANSFERASE_ | 12/374 | 215/18046 | 0.001821239 | 0.048727958 | 79709/440138/29880/79070/ |
| ACTIVITY_TRANSFERRING_ | | | | | 143888/79586/6388/23509/ |
| HEXOSYL_GROUPS | | | | | 55757/54480/23333/79053 |
| GO_PROTEIN_PEPTIDYL_ | 5/374 | 43/18046 | 0.001876107 | 0.049540462 | 10283/53938/23307/51661/ |
| PROLYL_ISOMERIZATION | | | | | 60681 |
| GO_EXORIBONUCLEASE_ | 4/374 | 26/18046 | 0.001891569 | 0.049540462 | 11340/56915/51010/23404 |
| COMPLEX | | | | | |
| GO_GAMMA_TUBULIN_ | 4/374 | 26/18046 | 0.001891569 | 0.049540462 | 10426/10844/55755/8481 |
| BINDING | | | | | |

TABLE 9J
TABLE OF CONTENTS

| Column | |
|---|---|
| Names | Description |
| Description | The name of the enriched GO term |
| GeneRatio | Shows the number of genes in cluster or virus interactome |
| | that match the term in Description and the full size of |
| | genes in the set considered in the enrichment analysis |
| BgRatio | Shows the number of genes annotated in the term and the |
| | total number of genes in the universe of annotations |
| pvalue | p-value resulting from a hypergeometric test for |
| | enrichment of genes |
| p.adjust | The adjusted p-value |
| geneID | Entrez Gene ID of the genes in cluster or virus |
| | interactome that match Description. There will be as |
| | many genes here as the numerator in GeneRatio. |

Table 9A-I list significantly enriched GO terms. Tables labeled as “Cluster_x” represent the results associated with clusters defined in FIG. 2A. Cluster 7 does not have a sheet as there were no terms with adjusted p-value < 0.05. Tables labeled as MERS, SARS-COV-1, and SARS-COV-2 represent the results associated with the high-confidence interactors of the corresponding virus.

Next, whether the conserved interactions were specific for certain viral proteins (FIG. 2C) was investigated, and it wasfound that some proteins (i.e., M, N, Nsp7/8/13) showed a disproportionately high fraction of shared interactions conserved across the three viruses. This suggests that the processes targeted by these proteins may be more essential and/or more likely to be required for other emerging coronaviruses. Such differences in conservation of interactions should be encoded, to some extent, in the degree of sequence differences. Comparing pairs of homologous proteins shared between SARS-CoV-2 and SARS-CoV-1 or MERS-CoV, a significant correlation was observed between sequence conservation and protein-protein interaction (PPI) similarity (calculated as Jaccard index) (FIG. 2D, r=0.58, p-value=0.0001). Without wishing to be bound by theoyr, this shows that the evolution of protein sequences strongly determines the divergence in the host interactors.

Referring to FIG. 2C, the percentage of interactions for each viral protein belonging to each cluster identified in FIG. 2A is shown.

Referring to FIG. 2D, a correlation between protein sequence similarity and PPI overlap (Jaccard index) comparing SARS-CoV-2 and SARS-CoV-1 (blue) or MERS-CoV (red) is shown. Interactions for PPI overlap are derived from the final thresholded list of interactions per virus.

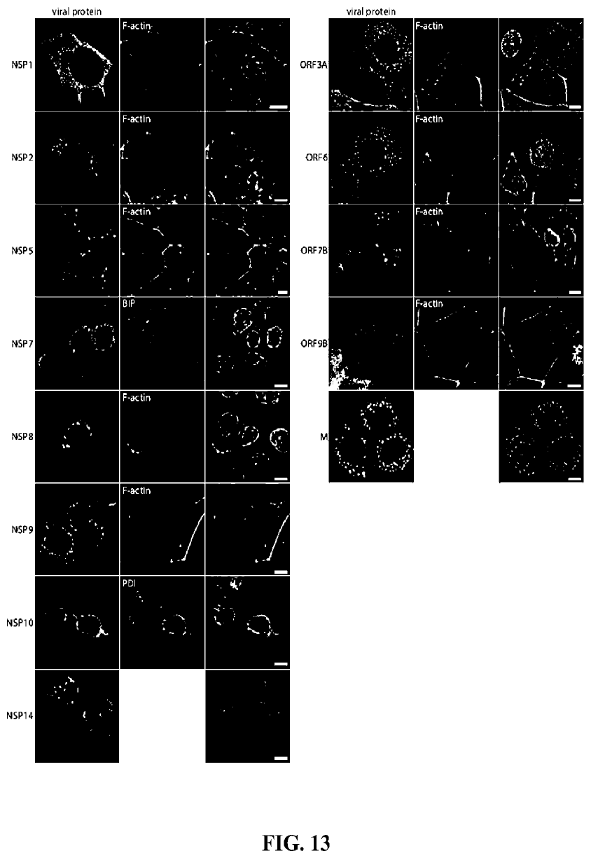

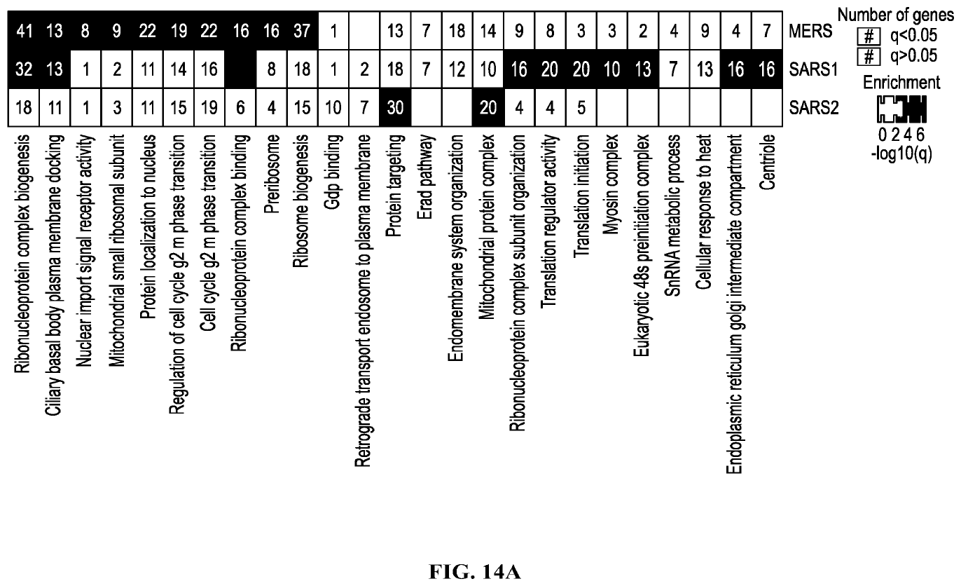

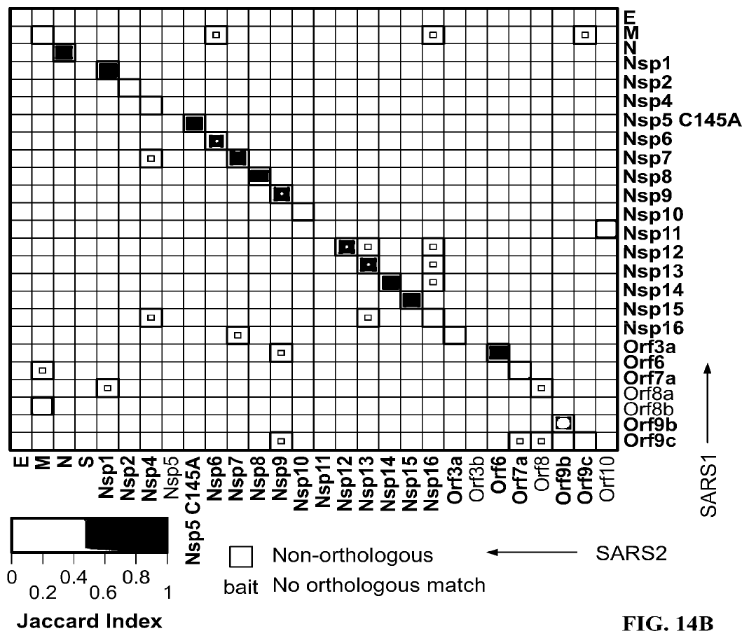

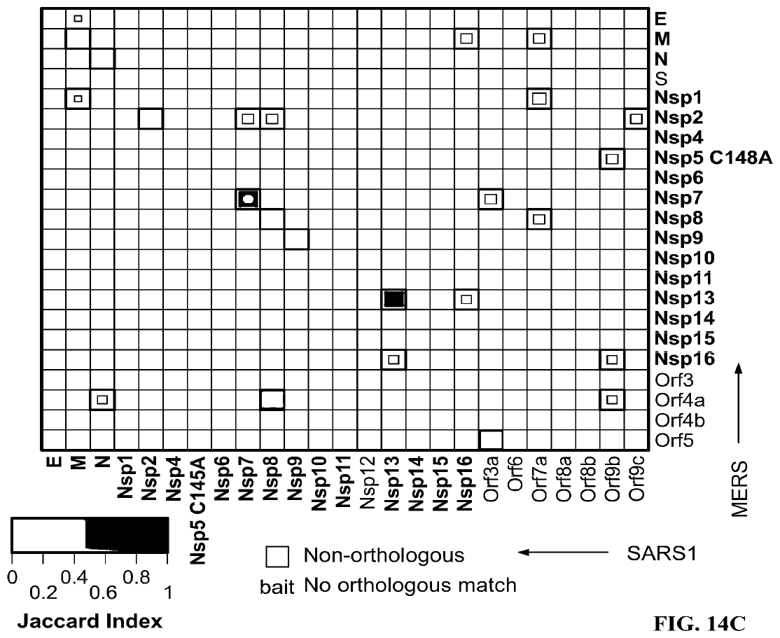

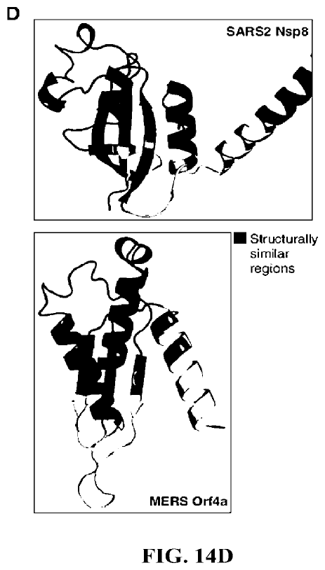

While studying the function of host proteins interacting with each virus it was noted that some shared cellular processes were targeted via different interactions across the viruses. To study this in more detail, the cellular processes significantly enriched in the interactomes of all three viruses (FIG. 14A and Table 9A-J) were identified, and ranked by the degree of overlapping proteins (FIG. 2E). This identified proteins related to the nuclear envelope, proteasomal catabolism, cellular response to heat, and regulation of intracellular protein transport as biological functions that are hijacked by these viruses through different human proteins. Additionally, it was found that up to 51% of protein interactions with a conserved human target occurred via a different (non-orthologous) viral protein (FIG. 2F) and, in some cases, the overlap of interactions for two non-orthologous virus baits was greater than that for the orthologous pair (FIG. 2G and FIG. 14B-C). For example, several interacting proteins of SARS-CoV-2 Nsp8 are also targeted by MERS-CoV Orf4a, and interactions of MERS-CoV Orf5 share interactors with SARS-CoV-2 Orf3a (FIG. 2G). In the case of Nsp8, some degree of structural homology was found between the C-terminal region of Nsp8 and a predicted structural model of Orf4a (FIG. 14D), indicative of a possible common interaction mechanism.

Referring to FIG. 2E, GO biological process terms significantly enriched (q<0.05) for all three virus PPIs with Jaccard index indicating overlap of genes from each term for pairwise comparisons between SARS-CoV-1 and SARS-CoV-2 (purple), SARS-CoV-1 and MERS-CoV (green) and SARS-CoV-2 and MERS-CoV (orange).

Referring to FIG. 2F, the fraction of shared preys between orthologous (blue) versus non-orthologous (red) viral protein baits is shown.

Referring to FIG. 2G, a heatmap depicting overlap in PPIs (Jaccard index) between each bait from SARS-CoV-2 and MERS-CoV is shown. Baits in grey were not assessed, do not exist, or do not have high-confidence interactors in the compared virus. Non-orthologous bait interactions are highlighted with a red square. GO=Gene Ontology; PPI=protein-protein interaction; SARS2=SARS-CoV-2; SARS1=SARS-CoV-1; MERS=MERS-CoV.

Referring to FIG. 14A, Gene Ontology (GO) enrichment analysis of the high-confidence interactors of the three viruses is shown. The top ten most significant terms are included per virus. Color indicates −log 10(q). Number indicates number of genes; white numbers denote significant enrichment (q<0.05), whereas grey numbers indicate non-significance (q>0.05).

Referring to FIG. 14B, a heatmap depicting overlap in protein-protein interactions (Jaccard index) between all baits from SARS-CoV-1 and SARS-CoV-2 is shown. Baits in grey were not assessed, do not exist, or do not have high-confidence interactors in the alternate virus. Nonorthologous baits are highlighted with a red square.

Referring to FIG. 14C, a heatmap depicting overlap in protein-protein interactions (Jaccard index) between all baits from SARS-CoV-1 and MERS-CoV is shown. Baits in grey were not assessed, do not exist, or do not have high-confidence interactors in the alternate virus. Non-orthologous baits are highlighted with a red square.

Referring to FIG. 14D, the structure of the C-terminal region of SARS-CoV-2 Nsp8 (upper panel) and a predicted structural model of MERS-CoV Orf4a (lower panel) is shown. Red represents structurally similar regions as determined by Geometricus.

In summary, it was found that sequence differences determine the degree of changes in viral-host interactions, and that often the same cellular process can be targeted via different viral and/or host proteins. Without wishing to be bound by theory, these results suggest some degree of plasticity in the way these viruses can control a given biological process in the host cell.

Quantitative Differential Interaction Scoring (DIS) Identifies Interactions Conserved Between Coronaviruses

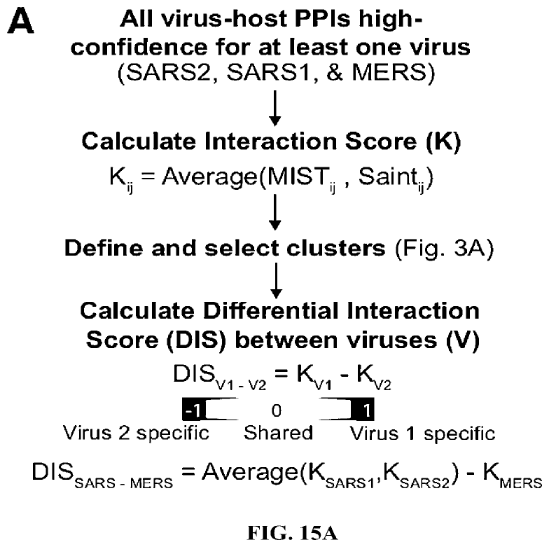

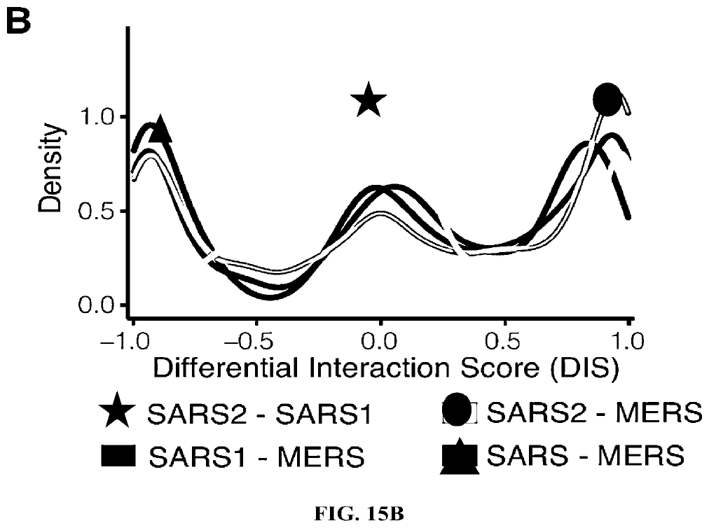

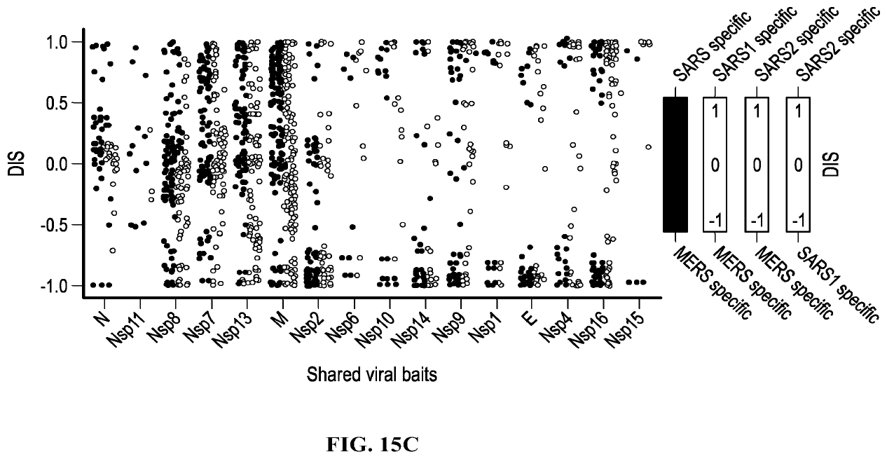

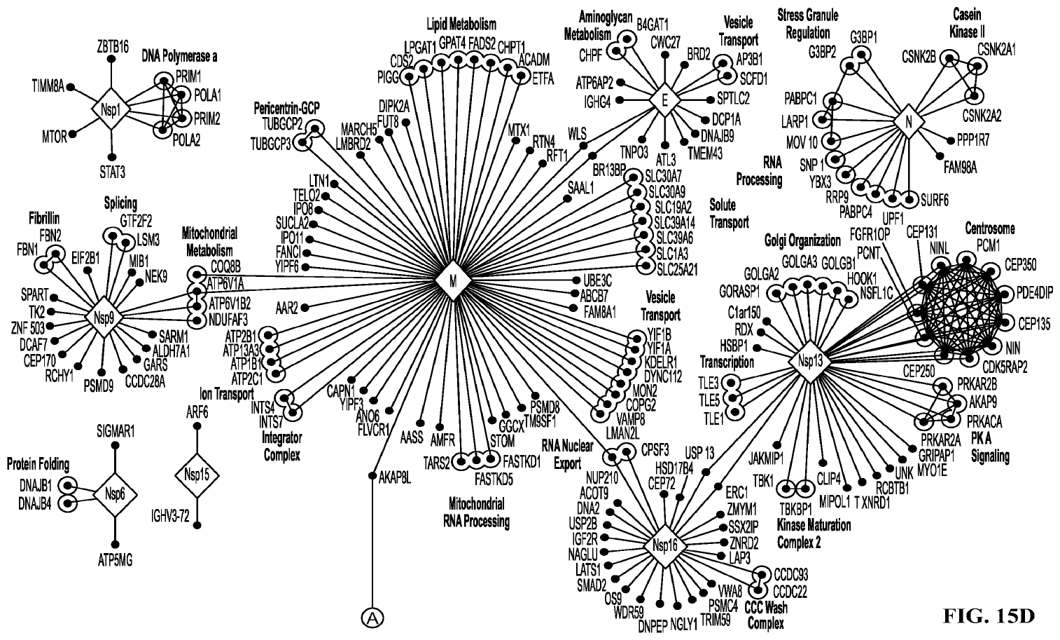

The identification of virus-host interactions conserved across pathogenic coronaviruses provides the opportunity to reveal host targets that may remain essential for these and other emerging coronaviruses. For a quantitative comparison of each virus-human interaction from viral baits shared by all three viruses, a differential interaction score (DIS) was developed. DIS is calculated between any pair of viruses and is defined as the difference between the interaction scores (K) from each virus (FIG. 15A and Table 10A-B). This kind of comparative analysis is beneficial as it permits the recovery of conserved interactions that may fall just below strict cutoffs. For each comparison, DIS was calculated for interactions residing in certain clusters as defined in the previous analysis (see FIG. 2A). For example, for the SARS-CoV-2 to MERS-CoV comparison, a DIS was computed for interactions residing in all clusters except cluster 3, where interactions are either not found or scores were very low for both SARS-CoV-2 and MERS-CoV. A DIS of 0 indicates that the interaction is confidently shared between the two viruses being compared, while a DIS of +1 or −1 indicates that the host protein interaction is specific for the virus listed first or second, respectively.

Referring to FIG. 15A, a flowchart depicting calculation of differential interactions scores (DIS) using the average between the Saint and MIST scores between every bait (i) and prey (j) to derive interaction score (K) is shown. The DIS is the difference between the interaction scores from each virus. The modified DIS (SARS-MERS) compares the average K from SARS-CoV-1 and SARS-CoV2 to that of MERS-CoV. Only viral bait proteins shared between all three viruses are included.

TABLE 10A

| Bait_Prey | Bait | Prey | MIST_MERS | MIST_SARS1 | MIST_SARS2 | Saint_MERS | Saint_SARS1 | Saint_SARS2 | BFDR_MERS | BFDR_SARS1 | BFDR_SARS2 |
|---|---|---|---|---|---|---|---|---|---|---|---|
| E-O00203 | E | AP3B1 | 0.2698 | 0.60657 | 0.963550095 | 0 | 0.63 | 0.99 | 0.75 | 0.1 | 0 |
| E-O15270 | E | SPTLC2 | 0.89523 | 0 | 0 | 0.97 | 0 | 0 | 0 | NA | NA |
| E-O43505 | E | B4GAT1 | 0.71348 | 0 | 0 | 1 | 0 | 0 | 0 | NA | NA |
| E-O60885 | E | BRD4 | 0.095039 | 0.68551 | 0.97848835 | 0 | 0 | 0.97 | 0.75 | 0.74 | 0 |
| E-O75787 | E | ATP6AP2 | 0.86035 | 0 | 0 | 0.98 | 0 | 0 | 0 | NA | NA |
| E-P01861 | E | IGHG4 | 0.99139 | 0 | 0 | 0.95 | 0 | 0 | 0.01 | NA | NA |
| E-P25440 | E | BRD2 | 0 | 0.36688 | 0.906592876 | 0 | 0.63 | 1 | NA | 0.12 | 0 |
| E-Q5T9L3 | E | WLS | 0.90131 | 0 | 0 | 0.95 | 0 | 0 | 0.01 | NA | NA |
| E-Q6DD88 | E | ATL3 | 0.98317 | 0 | 0 | 1 | 0 | 0 | 0 | NA | NA |
| E-Q6UX04 | E | CWC27 | 0.03892 | 0.65353 | 0.89310916 | 0 | 0.98 | 0.66 | 0.75 | 0 | 0.03 |
| E-Q86VM9 | E | ZC3H18 | 0 | 0.61758 | 0.796415039 | 0 | 0 | 0.97 | NA | 0.74 | 0 |
| E-Q8IWA5 | E | SLC44A2 | 0 | 0 | 0.950342834 | 0 | 0 | 0.98 | NA | NA | 0 |
| E-Q8IZ52 | E | CHPF | 0.80352 | 0 | 0 | 0.97 | 0 | 0 | 0.01 | NA | NA |
| E-Q8WVM8 | E | SCFD1 | 0.72135 | 0.30634 | 0 | 0.95 | 0 | 0 | 0.01 | 0.74 | NA |
| E-Q8WY22 | E | BRI3BP | 0.99124 | 0 | 0 | 1 | 0 | 0 | 0 | NA | NA |
| E-Q92665 | E | MRPS31 | 0 | 0.86696 | 0 | 0 | 0.95 | 0 | NA | 0.01 | NA |
| E-Q9BTV4 | E | TMEM43 | 0.87527 | 0 | 0 | 1 | 0 | 0 | 0 | NA | NA |
| E-Q9NPI6 | E | DCP1A | 0.97974 | 0 | 0 | 1 | 0 | 0 | 0 | NA | NA |
| E-Q9UBS3 | E | DNAJB9 | 0.97286 | 0 | 0 | 0.98 | 0 | 0 | 0 | NA | NA |
| E-Q9ULP9 | E | TBC1D24 | 0 | 0.91651 | 0 | 0 | 0.97 | 0 | NA | 0.01 | NA |
| E-Q9Y5L0 | E | TNPO3 | 0.90977 | 0 | 0 | 0.99 | 0 | 0 | 0 | NA | NA |
| M-O15321 | M | TM9SF1 | 0 | 0.99145 | 0.55254956 | 0 | 1 | 1 | NA | 0 | 0 |
| M-O15397 | M | IPO8 | 0.83073 | 0.70698 | 0.582052482 | 0.31 | 1 | 0.98 | 0.22 | 0 | 0 |
| M-O15431 | M | SLC31A1 | 0 | 0.74357 | 0.685510759 | 0 | 0.95 | 0 | NA | 0.01 | 0.69 |
| M-O43156 | M | TTI1 | 0 | 0.98681 | 0 | 0 | 0.97 | 0 | NA | 0.01 | NA |
| M-O60779 | M | SLC19A2 | 0 | 0.98935 | 0.744933284 | 0 | 0.97 | 0.32 | NA | 0.01 | 0.23 |
| M-O75027 | M | ABCB7 | 0 | 0.73924 | 0.598033368 | 0 | 1 | 0.65 | NA | 0 | 0.05 |
| M-O75439 | M | PMPCB | 0 | 0 | 0.985120198 | 0 | 0 | 1 | NA | NA | 0 |
| M-O94822 | M | LTN1 | 0.99367 | 0.92809 | 0.537310468 | 0.94 | 1 | 1 | 0.01 | 0 | 0 |
| M-O94829 | M | IPO13 | 0.66055 | 0.99269 | 0.586881917 | 0.31 | 1 | 0.33 | 0.22 | 0 | 0.19 |
| M-O95070 | M | YIF1A | 0 | 0.48186 | 0.856000835 | 0 | 0.65 | 0.97 | NA | 0.09 | 0 |
| M-O95674 | M | CDS2 | 0.98243 | 0.85794 | 0.529235842 | 0.96 | 1 | 1 | 0.01 | 0 | 0 |
| M-O95864 | M | FADS2 | 0 | 0.96971 | 0.587168157 | 0 | 0.98 | 0.65 | NA | 0 | 0.05 |
| M-P05026 | M | ATP1B1 | 0 | 0.99394 | 0.817625601 | 0 | 1 | 1 | NA | 0 | 0 |
| M-P07384 | M | CAPN1 | 0.63285 | 0.82648 | 0.463123411 | 0 | 1 | 0.99 | 0.75 | 0 | 0 |
| M-P11310 | M | ACADM | 0 | 0.29729 | 0.724348569 | 0 | 0.63 | 0.97 | NA | 0.1 | 0 |
| M-P13804 | M | ETFA | 0 | 0.47824 | 0.718398295 | 0 | 1 | 0.97 | NA | 0 | 0 |
| M-P20020 | M | ATP2B1 | 0.85897 | 0.88177 | 0.66909613 | 0.31 | 1 | 1 | 0.22 | 0 | 0 |
| M-P23634 | M | ATP2B4 | 0 | 0.94562 | 0.429226053 | 0 | 0.67 | 0.32 | NA | 0.04 | 0.23 |
| M-P24390 | M | KDELR1 | 0 | 0.72294 | 0.454194622 | 0 | 0.95 | 0.64 | NA | 0.01 | 0.08 |
| M-P27105 | M | STOM | 0 | 0.69334 | 0.752971772 | 0 | 0.98 | 0.98 | NA | 0 | 0 |
| M-P33527 | M | ABCC1 | 0 | 0.97041 | 0 | 0 | 1 | 0 | NA | 0 | NA |
| M-P35670 | M | ATP7B | 0 | 0.99058 | 0 | 0 | 0.98 | 0 | NA | 0 | NA |
| M-P38435 | M | GGCX | 0 | 0.93354 | 0.789966998 | 0 | 1 | 0.96 | NA | 0 | 0.01 |
| M-P38606 | M | ATP6V1A | 0 | 0.36314 | 0.794938493 | 0 | 0.98 | 0.65 | NA | 0 | 0.05 |
| M-P40763 | M | STAT3 | 0 | 0.87424 | 0 | 0 | 0.99 | 0 | NA | 0 | NA |
| M-P43003 | M | SLC1A3 | 0.97418 | 0.87471 | 0.688209246 | 0.31 | 1 | 0.98 | 0.22 | 0 | 0 |
| M-P48556 | M | PSMD8 | 0 | 0.37311 | 0.881424779 | 0 | 0.63 | 0.65 | NA | 0.1 | 0.05 |
| M-P49768 | M | PSEN1 | 0.98243 | 0.77968 | 0.538073775 | 0.31 | 0.98 | 0 | 0.22 | 0 | 0.69 |
| M-P56589 | M | PEX3 | 0.61637 | 0.78566 | 0 | 0 | 0.98 | 0 | 0.75 | 0 | NA |
| M-P61803 | M | DAD1 | 0 | 0.91673 | 0.544853165 | 0 | 0.99 | 0.32 | NA | 0 | 0.23 |
| M-P98194 | M | ATP2C1 | 0.98279 | 0.96438 | 0.437113101 | 0.62 | 1 | 1 | 0.09 | 0 | 0 |
| M-Q00765 | M | REEP5 | 0 | 0.30793 | 0.913088507 | 0 | 0.33 | 1 | NA | 0.22 | 0 |
| M-Q10713 | M | PMPCA | 0 | 0 | 0.991059815 | 0 | 0 | 1 | NA | NA | 0 |
| M-Q13409 | M | DYNC1I2 | 0 | 0.75358 | 0.685510754 | 0 | 0.98 | 0.33 | NA | 0 | 0.19 |
| M-Q13433 | M | SLC39A6 | 0.44339 | 0.92272 | 0.886153423 | 0.31 | 0.99 | 0.64 | 0.22 | 0 | 0.08 |
| M-Q13505 | M | MTX1 | 0 | 0.7196 | 0.750438714 | 0 | 0.98 | 0.64 | NA | 0 | 0.08 |
| M-Q14CZ7 | M | FASTKD3 | 0 | 0.99394 | 0.303183199 | 0 | 0.95 | 0 | NA | 0.01 | 0.69 |
| M-Q15043 | M | SLC39A14 | 0.18378 | 0.72087 | 0.537571222 | 0 | 1 | 1 | 0.75 | 0 | 0 |
| M-Q15386 | M | UBE3C | 0 | 0.70952 | 0.265922883 | 0 | 0.67 | 0.64 | NA | 0.04 | 0.08 |
| M-Q4KMQ2 | M | ANO6 | 0 | 0.86403 | 0.993904419 | 0 | 0.32 | 1 | NA | 0.28 | 0 |
| M-Q53R41 | M | FASTKD1 | 0.58836 | 0.8606 | 0.622957566 | 0.97 | 1 | 1 | 0 | 0 | 0 |
| M-Q5BJH7 | M | YIF1B | 0.37122 | 0.98935 | 0.597949548 | 0 | 0.97 | 1 | 0.75 | 0.01 | 0 |
| M-Q5H8A4 | M | PIGG | 0.13645 | 0.98937 | 0.558367337 | 0 | 1 | 0.97 | 0.75 | 0 | 0 |
| M-Q5JRX3 | M | PITRM1 | 0 | 0.0011109 | 0.952308232 | 0 | 0 | 1 | NA | 0.74 | 0 |
| M-Q5T1Q4 | M | SLC35F1 | 0 | 0.98681 | 0 | 0 | 0.97 | 0 | NA | 0.01 | NA |
| M-Q5T9L3 | M | WLS | 0.086274 | 0.99094 | 0.626982883 | 0 | 1 | 0.99 | 0.75 | 0 | 0 |
| M-Q68DH5 | M | LMBRD2 | 0.98693 | 0.68551 | 0.244942963 | 0.95 | 0 | 0 | 0.01 | 0.74 | 0.69 |
| M-Q6AI08 | M | HEATR6 | 0 | 0.82843 | 0 | 0 | 0.97 | 0 | NA | 0.01 | NA |
| M-Q6P3X3 | M | TTC27 | 0.74622 | 0.72081 | 0.362292246 | 1 | 1 | 0.33 | 0 | 0 | 0.19 |
| M-Q6PJG6 | M | BRAT1 | 0 | 0.99113 | 0 | 0 | 1 | 0 | NA | 0 | NA |
| M-Q6PML9 | M | SLC30A9 | 0 | 0.47111 | 0.886323242 | 0 | 0.66 | 0.65 | NA | 0.07 | 0.05 |
| M-Q7L8L6 | M | FASTKD5 | 0 | 0.71047 | 0.758365887 | 0 | 1 | 1 | NA | 0 | 0 |
| M-Q7RTS9 | M | DYM | 0 | 0.98935 | 0 | 0 | 0.97 | 0 | NA | 0.01 | NA |
| M-Q7Z3U7 | M | MON2 | 0 | 0.98147 | 0.685510175 | 0 | 0.98 | 0.32 | NA | 0 | 0.23 |
| M-Q86UL3 | M | GPAT4 | 0.29976 | 0.84955 | 0.48498957 | 0.31 | 1 | 0.96 | 0.22 | 0 | 0.01 |
| M-Q8N1F8 | M | STK11IP | 0 | 0.99394 | 0 | 0 | 0.95 | 0 | NA | 0.01 | NA |
| M-Q8N5G2 | M | MACO1 | 0 | 0.9356 | 0 | 0 | 0.67 | 0 | NA | 0.04 | NA |
| M-Q8NDZ4 | M | DIPK2A | 0.74768 | 0 | 0 | 1 | 0 | 0 | 0 | NA | NA |
| M-Q8NEW0 | M | SLC30A7 | 0.58339 | 0.62216 | 0.766972437 | 0.64 | 0.97 | 1 | 0.08 | 0.01 | 0 |
| M-Q8TBF5 | M | PIGX | 0 | 0.99009 | 0.427323161 | 0 | 0.99 | 0.33 | NA | 0 | 0.19 |
| M-Q8TCJ2 | M | STT3B | 0 | 0.99097 | 0.01779039 | 0 | 1 | 0 | NA | 0 | 0.69 |
| M-Q8TEM1 | M | NUP210 | 0.72584 | 0.029862 | 0 | 1 | 0 | 0 | 0 | 0.74 | NA |
| M-Q8WUD6 | M | CHPT1 | 0 | 0.89785 | 0.635974009 | 0 | 0.98 | 0.65 | NA | 0 | 0.05 |
| M-Q8WY22 | M | BRI3BP | 0 | 0.82488 | 0.574146705 | 0 | 1 | 1 | NA | 0 | 0 |
| M-Q92604 | M | LPGAT1 | 0 | 0.98681 | 0.652520995 | 0 | 0.97 | 0.66 | NA | 0.01 | 0.04 |
| M-Q92616 | M | GCN1 | 0.76728 | 0.54828 | 0 | 1 | 1 | 0 | 0 | 0 | NA |
| M-Q969V3 | M | NCLN | 0.48416 | 0.77626 | 0.464252443 | 1 | 1 | 0.32 | 0 | 0 | 0.23 |
| M-Q96AA3 | M | RFT1 | 0 | 0.80897 | 0.551265158 | 0 | 0.95 | 0.98 | NA | 0.01 | 0 |
| M-Q96CW5 | M | TUBGCP3 | 0.55409 | 0.99335 | 0.753607002 | 0.33 | 1 | 1 | 0.18 | 0 | 0 |
| M-Q96D53 | M | COQ8B | 0 | 0.94235 | 0.80074032 | 0 | 1 | 0.99 | NA | 0 | 0 |
| M-Q96EC8 | M | YIPF6 | 0.94049 | 0.97013 | 0.677288018 | 1 | 0.65 | 0.64 | 0 | 0.09 | 0.08 |
| M-Q96ER3 | M | SAAL1 | 0 | 0.37631 | 0.769472929 | 0 | 0.98 | 1 | NA | 0 | 0 |
| M-Q96HR9 | M | REEP6 | 0 | 0 | 0.955657163 | 0 | 0 | 0.65 | NA | NA | 0.05 |
| M-Q96HW7 | M | INTS4 | 0 | 0.81238 | 0.943304706 | 0 | 0.33 | 0.65 | NA | 0.21 | 0.05 |
| M-Q99805 | M | TM9SF2 | 0 | 0.79474 | 0.410099202 | 0 | 0.67 | 0.33 | NA | 0.04 | 0.19 |
| M-Q9BQ95 | M | ECSIT | 0 | 0.98935 | 0 | 0 | 0.97 | 0 | NA | 0.01 | NA |
| M-Q9BQT8 | M | SLC25A21 | 0.43267 | 0.69462 | 0.880779937 | 0 | 0.65 | 0.65 | 0.75 | 0.09 | 0.05 |
| M-Q9BSJ2 | M | TUBGCP2 | 0.89421 | 0.94558 | 0.83958055 | 0.97 | 1 | 1 | 0 | 0 | 0 |
| M-Q9BTY2 | M | FUCA2 | 0 | 0.91171 | 0.440518376 | 0 | 0.98 | 0.32 | NA | 0 | 0.23 |
| M-Q9BV40 | M | VAMP8 | 0.98738 | 0 | 0 | 1 | 0 | 0 | 0 | NA | NA |
| M-Q9BW92 | M | TARS2 | 0.061949 | 0.37463 | 0.758110505 | 0 | 1 | 0.97 | 0.75 | 0 | 0 |
| M-Q9BYC5 | M | FUT8 | 0.963 | 0 | 0 | 0.98 | 0 | 0 | 0 | NA | NA |
| M-Q9C0D9 | M | SELENOI | 0 | 0.98935 | 0.879776538 | 0 | 0.97 | 0 | NA | 0.01 | 0.69 |
| M-Q9C0E2 | M | XPO4 | 0 | 0.94301 | 0.879776036 | 0 | 0.97 | 0 | NA | 0.01 | 0.69 |
| M-Q9GZM5 | M | YIPF3 | 0.53419 | 0.92485 | 0.483341368 | 0 | 0.98 | 0.65 | 0.75 | 0 | 0.05 |
| M-Q9H0V9 | M | LMAN2L | 0.97612 | 0 | 0 | 0.98 | 0 | 0 | 0 | NA | NA |
| M-Q9H2J7 | M | SLC6A15 | 0 | 0.99394 | 0.246796903 | 0 | 0.99 | 0 | NA | 0 | 0.69 |
| M-Q9H583 | M | HEATR1 | 0.70638 | 0.75713 | 0 | 0.99 | 1 | 0 | 0 | 0 | NA |
| M-Q9H7F0 | M | ATP13A3 | 0 | 0.99199 | 0.487611844 | 0 | 1 | 0.97 | NA | 0 | 0 |
| M-Q9H845 | M | ACAD9 | 0 | 0.84516 | 0 | 0 | 1 | 0 | NA | 0 | NA |
| M-Q9H8M5 | M | CNNM2 | 0 | 0.99394 | 0 | 0 | 0.99 | 0 | NA | 0 | NA |
| M-Q9NQC3 | M | RTN4 | 0 | 0.44481 | 0.873826097 | 0 | 1 | 1 | NA | 0 | 0 |
| M-Q9NVH2 | M | INTS7 | 0 | 0.89434 | 0.808244829 | 0 | 0.97 | 0.64 | NA | 0.01 | 0.08 |
| M-Q9NVI1 | M | FANCI | 0.81327 | 0.72447 | 0.557293884 | 1 | 1 | 1 | 0 | 0 | 0 |
| M-Q9NX47 | M | MARCH5 | 0.98243 | 0 | 0 | 0.99 | 0 | 0 | 0 | NA | NA |
| M-Q9P2R7 | M | SUCLA2 | 0.66214 | 0.76644 | 0.419797298 | 0.95 | 1 | 0.98 | 0.01 | 0 | 0 |
| M-Q9UBF2 | M | COPG2 | 0 | 0.91857 | 0.117335394 | 0 | 1 | 0.99 | NA | 0 | 0 |
| M-Q9UBU6 | M | FAM8A1 | 0 | 0.88005 | 0.80448832 | 0 | 0.63 | 0.97 | NA | 0.1 | 0 |
| M-Q9UDR5 | M | AASS | 0 | 0.95492 | 0.765109504 | 0 | 0.65 | 0.98 | NA | 0.08 | 0 |
| M-Q9UI26 | M | IPO11 | 0.99367 | 0.68215 | 0.649385462 | 0.99 | 1 | 1 | 0 | 0 | 0 |
| M-Q9UKV5 | M | AMFR | 0.27192 | 0.98708 | 0.043516186 | 0 | 1 | 1 | 0.75 | 0 | 0 |
| M-Q9ULF5 | M | SLC39A10 | 0 | 0.73747 | 0 | 0 | 1 | 0 | NA | 0 | NA |
| M-Q9ULX6 | M | AKAP8L | 0 | 0.34 | 0.751981385 | 0 | 0.98 | 1 | NA | 0 | 0 |
| M-Q9Y312 | M | AAR2 | 0.56081 | 0.48301 | 0.801486724 | 0.31 | 0.66 | 0.99 | 0.22 | 0.05 | 0 |
| M-Q9Y4R8 | M | TELO2 | 0.74925 | 0.91945 | 0.542406748 | 1 | 1 | 1 | 0 | 0 | 0 |
| M-Q9Y5Y0 | M | FLVCR1 | 0 | 0.97851 | 0.640982121 | 0 | 0.98 | 0.65 | NA | 0 | 0.05 |
| M-Q9Y6E2 | M | BZW2 | 0 | 0 | 0.756364362 | 0 | 0 | 0.97 | NA | NA | 0 |
| N-O43818 | N | RRP9 | 0.54769 | 0.90021 | 0.861168798 | 1 | 1 | 1 | 0 | 0 | 0 |
| N-O75683 | N | SURF6 | 0.45451 | 0.70857 | 0.608432617 | 0.98 | 1 | 0.99 | 0 | 0 | 0 |
| N-P11940 | N | PABPC1 | 0.48869 | 0.64471 | 0.736635929 | 1 | 1 | 1 | 0 | 0 | 0 |
| N-P16989 | N | YBX3 | 0.40553 | 0.74013 | 0.654394207 | 0.62 | 1 | 1 | 0.09 | 0 | 0 |
| N-P19784 | N | CSNK2A2 | 0.76302 | 0.78377 | 0.875048268 | 1 | 1 | 1 | 0 | 0 | 0 |
| N-P67870 | N | CSNK2B | 0.52768 | 0.70614 | 0.803607895 | 0.61 | 1 | 0.97 | 0.12 | 0 | 0 |
| N-P68400 | N | CSNK2A1 | 0.87167 | 0.64361 | 0.981288441 | 1 | 0.99 | 0.32 | 0 | 0 | 0.23 |
| N-Q13283 | N | G3BP1 | 0 | 0.92369 | 0.95331626 | 0 | 1 | 1 | NA | 0 | 0 |
| N-Q13310 | N | PABPC4 | 0.52068 | 0.86606 | 0.846200046 | 1 | 1 | 1 | 0 | 0 | 0 |
| N-Q15435 | N | PPP1R7 | 0.98385 | 0 | 0 | 1 | 0 | 0 | 0 | NA | NA |
| N-Q6PKG0 | N | LARP1 | 0.512 | 0.742 | 0.73787466 | 1 | 1 | 1 | 0 | 0 | 0 |
| N-Q86U42 | N | PABPN1 | 0.45331 | 0.71046 | 0.534817993 | 0.31 | 0.95 | 0.32 | 0.22 | 0.01 | 0.31 |
| N-Q8NCA5 | N | FAM98A | 0.53223 | 0.9296 | 0.921076719 | 0.64 | 1 | 1 | 0.08 | 0 | 0 |
| N-Q8TAD8 | N | SNIP1 | 0.65313 | 0.71644 | 0.818230245 | 0.88 | 1 | 1 | 0.02 | 0 | 0 |
| N-Q92900 | N | UPF1 | 0.11167 | 0.51968 | 0.753067271 | 0 | 0.97 | 1 | 0.75 | 0.01 | 0 |
| N-Q9BQ75 | N | CMSS1 | 0.47647 | 0.83768 | 0.415963465 | 0.94 | 1 | 0 | 0.01 | 0 | 0.69 |
| N-Q9HCE1 | N | MOV10 | 0.66104 | 0.61115 | 0.736672944 | 1 | 0.97 | 0.99 | 0 | 0.01 | 0 |
| N-Q9UN86 | N | G3BP2 | 0 | 0.87669 | 0.958133672 | 0 | 1 | 1 | NA | 0 | 0 |
| nsp1-O60220 | nsp1 | TIMM8A | 0.70557 | 0 | 0 | 1 | 0 | 0 | 0 | NA | NA |
| nsp1-P09884 | nsp1 | POLA1 | 0 | 0.68551 | 0.981264591 | 0 | 1 | 0.99 | NA | 0 | 0 |
| nsp1-P40763 | nsp1 | STAT3 | 0.9586 | 0 | 0 | 0.99 | 0 | 0 | 0 | NA | NA |
| nsp1-P42345 | nsp1 | MTOR | 0.94974 | 0 | 0 | 0.67 | 0 | 0 | 0.04 | NA | NA |
| nsp1-P49642 | nsp1 | PRIM1 | 0 | 0.65454 | 0.981268688 | 0 | 0.99 | 0.99 | NA | 0 | 0 |
| nsp1-P49643 | nsp1 | PRIM2 | 0 | 0.649 | 0.993975192 | 0 | 1 | 1 | NA | 0 | 0 |
| nsp1-Q05516 | nsp1 | ZBTB16 | 0.98489 | 0 | 0 | 1 | 0 | 0 | 0 | NA | NA |
| nsp1-Q14181 | nsp1 | POLA2 | 0 | 0.99329 | 0.943678488 | 0 | 1 | 0.67 | NA | 0 | 0.03 |
| nsp1-Q8NBJ5 | nsp1 | COLGALT1 | 0 | 0 | 0.794123974 | 0 | 0 | 1 | NA | NA | 0 |
| nsp1-Q99959 | nsp1 | PKP2 | 0 | 0 | 0.964585351 | 0 | 0 | 1 | NA | NA | 0 |
| nsp10-O94973 | nsp10 | AP2A2 | 0 | 0.77587 | 0.99112813 | 0 | 0.66 | 1 | NA | 0.06 | 0 |
| nsp10-P28330 | nsp10 | ACADL | 0.88002 | 0 | 0 | 1 | 0 | 0 | 0 | NA | NA |
| nsp10-P55789 | nsp10 | GFER | 0 | 0.46503 | 0.965372815 | 0 | 0.41 | 1 | NA | 0.17 | 0 |
| nsp10-Q6Q0C0 | nsp10 | TRAF7 | 0 | 0.98559 | 0.993045461 | 0 | 1 | 0 | NA | 0 | 0.69 |
| nsp10-Q969X5 | nsp10 | ERGIC1 | 0 | 0.86515 | 0.912239515 | 0 | 1 | 1 | NA | 0 | 0 |
| nsp10-Q96CW1 | nsp10 | AP2M1 | 0 | 0.74596 | 0.982905884 | 0 | 0.33 | 0.98 | NA | 0.24 | 0 |
| nsp10-Q9BZH6 | nsp10 | WDR11 | 0.97455 | 0 | 0 | 1 | 0 | 0 | 0 | NA | NA |
| nsp10-Q9C026 | nsp10 | TRIM9 | 0.89351 | 0 | 0 | 0.66 | 0 | 0 | 0.05 | NA | NA |
| nsp10-Q9HAV7 | nsp10 | GRPEL1 | 0 | 0.53137 | 0.986587081 | 0 | 0.99 | 0.98 | NA | 0 | 0 |
| nsp11-O14734 | nsp11 | ACOT8 | 0.70954 | 0.3104 | 0.369791477 | 0.96 | 0.33 | 0.33 | 0.01 | 0.2 | 0.18 |
| nsp11-O75347 | nsp11 | TBCA | 0.47761 | 0.47563 | 0.768344701 | 0.78 | 0.67 | 0.93 | 0.03 | 0.05 | 0.01 |
| nsp11-Q92624 | nsp11 | APPBP2 | 0.64641 | 0.85506 | 0.941018639 | 0.62 | 1 | 0.33 | 0.09 | 0 | 0.19 |
| nsp11-Q9C0D3 | nsp11 | ZYG11B | 0 | 0.89544 | 0.447833969 | 0 | 1 | 1 | NA | 0 | 0 |
| nsp13-A7MCY6 | nsp13 | TBKBP1 | 0.68551 | 0.86537 | 0.985289524 | 0 | 0.32 | 1 | 0.75 | 0.28 | 0 |
| nsp13-O14578 | nsp13 | CIT | 0 | 0 | 0.887314876 | 0 | 0 | 1 | NA | NA | 0 |
| nsp13-O14639 | nsp13 | ABLIM1 | 0 | 0.74788 | 0 | 0 | 1 | 0 | NA | 0 | NA |
| nsp13-O14908 | nsp13 | GIPC1 | 0.22076 | 0.87091 | 0 | 0 | 0.98 | 0 | 0.75 | 0 | NA |
| nsp13-O60237 | nsp13 | PPP1R12B | 0.22137 | 0.74867 | 0 | 0.31 | 0.67 | 0 | 0.22 | 0.04 | NA |
| nsp13-O60784 | nsp13 | TOM1 | 0.39582 | 0.81982 | 0.196041465 | 0.64 | 1 | 0.33 | 0.07 | 0 | 0.18 |
| nsp13-O75381 | nsp13 | PEX14 | 0.68551 | 0.87952 | 0 | 0.31 | 0.66 | 0 | 0.22 | 0.05 | NA |
| nsp13-O75506 | nsp13 | HSBP1 | 0 | 0.52758 | 0.851502614 | 0 | 0.99 | 1 | NA | 0 | 0 |
| nsp13-O95613 | nsp13 | PCNT | 0.95289 | 0.95032 | 0.971855938 | 1 | 1 | 1 | 0 | 0 | 0 |
| nsp13-O95684 | nsp13 | FGFR1OP | 0.68551 | 0.86156 | 0.981570359 | 0 | 0.67 | 0.65 | 0.75 | 0.05 | 0.05 |
| nsp13-P06396 | nsp13 | GSN | 0.29922 | 0.74995 | 0 | 0.33 | 1 | 0 | 0.18 | 0 | NA |
| nsp13-P09493 | nsp13 | TPM1 | 0.76988 | 0.81095 | 0.197572818 | 1 | 1 | 0.33 | 0 | 0 | 0.18 |
| nsp13-P13861 | nsp13 | PRKAR2A | 0.87649 | 0.79998 | 0.897857211 | 1 | 1 | 1 | 0 | 0 | 0 |
| nsp13-P14649 | nsp13 | MYL6B | 0.77192 | 0.85675 | 0.303981322 | 0.98 | 1 | 0.33 | 0 | 0 | 0.18 |
| nsp13-P17612 | nsp13 | PRKACA | 0.84509 | 0.86768 | 0.880321174 | 0.98 | 1 | 1 | 0 | 0 | 0 |
| nsp13-P28289 | nsp13 | TMOD1 | 0.414 | 0.71944 | 0.139654825 | 0.66 | 1 | 0.33 | 0.05 | 0 | 0.18 |
| nsp13-P31323 | nsp13 | PRKAR2B | 0.98498 | 0.88015 | 0.983191506 | 0.97 | 0.66 | 1 | 0 | 0.07 | 0 |
| nsp13-P35241 | nsp13 | RDX | 0 | 0.86694 | 0.912028315 | 0 | 0.97 | 1 | NA | 0.01 | 0 |
| nsp13-P49454 | nsp13 | CENPF | 0.91284 | 0.88015 | 0.873840643 | 0.97 | 0 | 1 | 0 | 0.74 | 0 |
| nsp13-P67936 | nsp13 | TPM4 | 0.86851 | 0.88611 | 0.381089268 | 1 | 1 | 0.33 | 0 | 0 | 0.18 |
| nsp13-Q04724 | nsp13 | TLE1 | 0 | 0.95538 | 0.96917283 | 0 | 0.98 | 1 | NA | 0 | 0 |
| nsp13-Q04726 | nsp13 | TLE3 | 0 | 0.85217 | 0.933626993 | 0 | 1 | 1 | NA | 0 | 0 |
| nsp13-Q08117 | nsp13 | TLE5 | 0 | 0.94933 | 0.962431031 | 0 | 0.65 | 0.66 | NA | 0.09 | 0.04 |
| nsp13-Q08378 | nsp13 | GOLGA3 | 0.90861 | 0.88663 | 0.928738823 | 1 | 1 | 1 | 0 | 0 | 0 |
| nsp13-Q08379 | nsp13 | GOLGA2 | 0.91185 | 0.90103 | 0.952311087 | 1 | 1 | 1 | 0 | 0 | 0 |
| nsp13-Q12965 | nsp13 | MYO1E | 0.87848 | 0.98702 | 0.685511322 | 1 | 1 | 0.33 | 0 | 0 | 0.18 |
| nsp13-Q13045 | nsp13 | FLII | 0.40852 | 0.74106 | 0.041584009 | 0.67 | 1 | 0.32 | 0.04 | 0 | 0.23 |
| nsp13-Q14789 | nsp13 | GOLGB1 | 0.85988 | 0.88008 | 0.985604541 | 0.31 | 1 | 1 | 0.22 | 0 | 0 |
| nsp13-Q15154 | nsp13 | PCM1 | 0.70364 | 0.75293 | 0.696288454 | 1 | 1 | 1 | 0 | 0 | 0 |
| nsp13-Q16881 | nsp13 | TXNRD1 | 0.96667 | 0 | 0 | 1 | 0 | 0 | 0 | NA | NA |
| nsp13-Q4V328 | nsp13 | GRIPAP1 | 0.87985 | 0.68552 | 0.989815969 | 0 | 1 | 1 | 0.75 | 0 | 0 |
| nsp13-Q5VT06 | nsp13 | CEP350 | 0.30194 | 0.73848 | 0.86755993 | 0.33 | 0.67 | 1 | 0.19 | 0.04 | 0 |
| nsp13-Q5VU43 | nsp13 | PDE4DIP | 0.98858 | 0.87932 | 0.979124391 | 1 | 1 | 1 | 0 | 0 | 0 |
| nsp13-Q5VUJ6 | nsp13 | LRCH2 | 0 | 0.7652 | 0 | 0 | 0.97 | 0 | NA | 0.01 | NA |
| nsp13-Q66GS9 | nsp13 | CEP135 | 0.8678 | 0.95899 | 0.975292134 | 0.66 | 0.98 | 1 | 0.05 | 0 | 0 |
| nsp13-Q6ZVM7 | nsp13 | TOM1L2 | 0.47294 | 0.92681 | 0.28330576 | 0 | 1 | 0.32 | 0.75 | 0 | 0.23 |
| nsp13-Q76N32 | nsp13 | CEP68 | 0.832 | 0 | 0.879704216 | 0.33 | 0 | 0.67 | 0.19 | NA | 0.03 |
| nsp13-Q7Z406 | nsp13 | MYH14 | 0.54878 | 0.70986 | 0.079233549 | 1 | 1 | 0.33 | 0 | 0 | 0.17 |
| nsp13-Q7Z7A1 | nsp13 | CNTRL | 0 | 0 | 0.989917408 | 0 | 0 | 1 | NA | NA | 0 |
| nsp13-Q8IUD2 | nsp13 | ERC1 | 0.98713 | 0.90874 | 0.990718127 | 1 | 0.66 | 1 | 0 | 0.05 | 0 |
| nsp13-Q8IWJ2 | nsp13 | GCC2 | 0.91146 | 0 | 0.987387119 | 0.98 | 0 | 1 | 0 | NA | 0 |
| nsp13-Q8N3C7 | nsp13 | CLIP4 | 0 | 0.90389 | 0.966944672 | 0 | 0.65 | 0.99 | NA | 0.08 | 0 |
| nsp13-Q8N4C6 | nsp13 | NIN | 0.98681 | 0.68551 | 0.991583194 | 1 | 1 | 1 | 0 | 0 | 0 |
| nsp13-Q8N8E3 | nsp13 | CEP112 | 0.84889 | 0.68551 | 0.964318835 | 0.33 | 0 | 0.65 | 0.19 | 0.74 | 0.05 |
| nsp13-Q8NDN9 | nsp13 | RCBTB1 | 0.78594 | 0 | 0 | 0.99 | 0 | 0 | 0 | NA | NA |
| nsp13-Q8TD10 | nsp13 | MIPOL1 | 0.88012 | 0.86835 | 0.98176996 | 1 | 1 | 1 | 0 | 0 | 0 |
| nsp13-Q8WXW3 | nsp13 | PIBF1 | 0.59305 | 0.83029 | 0.610504389 | 0 | 0.67 | 0 | 0.75 | 0.04 | 0.69 |
| nsp13-Q92614 | nsp13 | MYO18A | 0.52971 | 0.87674 | 0.152846567 | 1 | 1 | 0.33 | 0 | 0 | 0.18 |
| nsp13-Q92995 | nsp13 | USP13 | 0.8682 | 0.96538 | 0.987514452 | 0.31 | 0.98 | 1 | 0.22 | 0 | 0 |
| nsp13-Q96CN9 | nsp13 | GCC1 | 0 | 0.65419 | 0.873361571 | 0 | 0 | 1 | NA | 0.74 | 0 |
| nsp13-Q96II8 | nsp13 | LRCH3 | 0.3371 | 0.90876 | 0 | 0.33 | 1 | 0 | 0.18 | 0 | NA |
| nsp13-Q96N16 | nsp13 | JAKMIP1 | 0 | 0.97246 | 0.987966991 | 0 | 1 | 1 | NA | 0 | 0 |
| nsp13-Q96SN8 | nsp13 | CDK5RAP2 | 0.9235 | 0.90815 | 0.939307247 | 1 | 1 | 1 | 0 | 0 | 0 |
| nsp13-Q99996 | nsp13 | AKAP9 | 0.98986 | 0.87708 | 0.990813809 | 1 | 1 | 1 | 0 | 0 | 0 |
| nsp13-Q9BQQ3 | nsp13 | GORASP1 | 0.98092 | 0.96911 | 0.986870312 | 0.31 | 0.99 | 1 | 0.22 | 0 | 0 |
| nsp13-Q9BQS8 | nsp13 | FYCO1 | 0.97192 | 0 | 0.733173301 | 1 | 0 | 0.65 | 0 | NA | 0.05 |
| nsp13-Q9BV19 | nsp13 | C1orf50 | 0 | 0.98609 | 0.932056845 | 0 | 0.95 | 1 | NA | 0.01 | 0 |
| nsp13-Q9BV73 | nsp13 | CEP250 | 0.87853 | 0.97667 | 0.990717833 | 1 | 1 | 1 | 0 | 0 | 0 |
| nsp13-Q9BZF9 | nsp13 | UACA | 0.5526 | 0.81512 | 0.431068209 | 0.65 | 1 | 0.33 | 0.06 | 0 | 0.18 |
| nsp13-Q9C0B0 | nsp13 | UNK | 0.97076 | 0 | 0 | 0.97 | 0 | 0 | 0 | NA | NA |
| nsp13-Q9H0E2 | nsp13 | TOLLIP | 0.66286 | 0.85198 | 0.148955029 | 0.67 | 1 | 0 | 0.05 | 0 | 0.69 |
| nsp13-Q9UHD2 | nsp13 | TBK1 | 0.68551 | 0.86537 | 0.993970596 | 0 | 0.32 | 1 | 0.75 | 0.28 | 0 |
| nsp13-Q9UJC3 | nsp13 | HOOK1 | 0.85988 | 0.68551 | 0.994048081 | 0.31 | 1 | 1 | 0.22 | 0 | 0 |
| nsp13-Q9ULV0 | nsp13 | MYO5B | 0 | 0.72441 | 0 | 0 | 0.67 | 0 | NA | 0.04 | NA |
| nsp13-Q9UM54 | nsp13 | MYO6 | 0.69034 | 0.77867 | 0.178240322 | 1 | 1 | 0.33 | 0 | 0 | 0.17 |
| nsp13-Q9UNZ2 | nsp13 | NSFL1C | 0.98824 | 0 | 0 | 0.95 | 0 | 0 | 0.01 | NA | NA |
| nsp13-Q9UPN4 | nsp13 | CEP131 | 0.69689 | 0.85879 | 0.583168141 | 1 | 1 | 0.99 | 0 | 0 | 0 |
| nsp13-Q9UPQ0 | nsp13 | LIMCH1 | 0 | 0.89548 | 0 | 0 | 1 | 0 | NA | 0 | NA |
| nsp13-Q9Y216 | nsp13 | NINL | 0.98456 | 0.68551 | 0.987790569 | 1 | 1 | 1 | 0 | 0 | 0 |
| nsp13-Q9Y411 | nsp13 | MYO5A | 0.60089 | 0.78808 | 0.199600266 | 0.98 | 1 | 0.33 | 0 | 0 | 0.18 |
| nsp13-Q9Y608 | nsp13 | LRRFIP2 | 0.61069 | 0.77317 | 0.182792533 | 0.98 | 1 | 0.33 | 0 | 0 | 0.18 |
| nsp14-O95071 | nsp14 | UBR5 | 0.75799 | 0 | 0 | 0.67 | 0 | 0 | 0.04 | NA | NA |
| nsp14-O95714 | nsp14 | HERC2 | 0 | 0.97816 | 0 | 0 | 1 | 0 | NA | 0 | NA |
| nsp14-P04637 | nsp14 | TP53 | 0.81292 | 0 | 0 | 1 | 0 | 0 | 0 | NA | NA |
| nsp14-P06280 | nsp14 | GLA | 0 | 0.80341 | 0.841137578 | 0 | 1 | 1 | NA | 0 | 0 |
| nsp14-P12268 | nsp14 | IMPDH2 | 0.73398 | 0.71448 | 0.989667608 | 0.64 | 0.97 | 1 | 0.08 | 0.01 | 0 |
| nsp14-P30153 | nsp14 | PPP2R1A | 0.72375 | 0.2207 | 0.433732356 | 1 | 0.18 | 0.72 | 0 | 0.43 | 0.02 |
| nsp14-P49959 | nsp14 | MRE11 | 0.78836 | 0 | 0 | 1 | 0 | 0 | 0 | NA | NA |
| nsp14-P63151 | nsp14 | PPP2R2A | 0.7599 | 0.44327 | 0.365051744 | 0.99 | 0.25 | 0 | 0 | 0.38 | 0.69 |
| nsp14-Q5QP82 | nsp14 | DCAF10 | 0.9884 | 0 | 0 | 1 | 0 | 0 | 0 | NA | NA |
| nsp14-Q5T9A4 | nsp14 | ATAD3B | 0.73349 | 0 | 0 | 1 | 0 | 0 | 0 | NA | NA |
| nsp14-Q92878 | nsp14 | RAD50 | 0.90053 | 0 | 0 | 1 | 0 | 0 | 0 | NA | NA |
| nsp14-Q96EN8 | nsp14 | MOCOS | 0.99187 | 0 | 0 | 1 | 0 | 0 | 0 | NA | NA |
| nsp14-Q96JN8 | nsp14 | NEURL4 | 0 | 0.87704 | 0 | 0 | 1 | 0 | NA | 0 | NA |
| nsp14-Q9NQX3 | nsp14 | GPHN | 0.84378 | 0 | 0 | 1 | 0 | 0 | 0 | NA | NA |
| nsp14-Q9NXA8 | nsp14 | SIRT5 | 0 | 0.99078 | 0.99363281 | 0 | 1 | 1 | NA | 0 | 0 |
| nsp15- | nsp15 | IGHV3-72 | 0.9363 | 0 | 0 | 1 | 0 | 0 | 0 | NA | NA |
| A0A0B4J1Y9 | | | | | | | | | | | |
| nsp15-P61970 | nsp15 | NUTF2 | 0 | 0 | 0.987886 | 0 | 0 | 0.97 | NA | NA | 0 |
| nsp15-P62330 | nsp15 | ARF6 | 0 | 0.713 | 0.988131492 | 0 | 1 | 1 | NA | 0 | 0 |
| nsp15-Q9H4P4 | nsp15 | RNF41 | 0 | 0 | 0.993560817 | 0 | 0 | 1 | NA | NA | 0 |
| nsp16-A3KMH1 | nsp16 | VWA8 | 0.72836 | 0 | 0 | 0.97 | 0 | 0 | 0 | NA | NA |
| nsp16-O14972 | nsp16 | VPS26C | 0 | 0 | 0.989672314 | 0 | 0 | 0.97 | NA | NA | 0.01 |
| nsp16-O43933 | nsp16 | PEX1 | 0 | 0 | 0.993038775 | 0 | 0 | 1 | NA | NA | 0 |
| nsp16-O60232 | nsp16 | ZNRD2 | 0.23358 | 0.73317 | 0.459525316 | 0.02 | 0.88 | 0.88 | 0.54 | 0.01 | 0.01 |
| nsp16-O60826 | nsp16 | CCDC22 | 0 | 0.55155 | 0.992439461 | 0 | 0.99 | 1 | NA | 0 | 0 |
| nsp16-O75382 | nsp16 | TRIM3 | 0 | 0 | 0.939078269 | 0 | 0 | 1 | NA | NA | 0 |
| nsp16-O75564 | nsp16 | JRK | 0 | 0 | 0.708146128 | 0 | 0 | 0.98 | NA | NA | 0 |
| nsp16-O75665 | nsp16 | OFD1 | 0 | 0 | 0.993704543 | 0 | 0 | 1 | NA | NA | 0 |
| nsp16-O95714 | nsp16 | HERC2 | 0 | 0 | 0.872117541 | 0 | 0 | 1 | NA | NA | 0 |
| nsp16-O95754 | nsp16 | SEMA4F | 0 | 0 | 0.990804706 | 0 | 0 | 1 | NA | NA | 0 |
| nsp16-O95835 | nsp16 | LATS1 | 0.82894 | 0 | 0 | 0.94 | 0 | 0 | 0.01 | NA | NA |
| nsp16-P11717 | nsp16 | IGF2R | 0.87428 | 0 | 0 | 0.97 | 0 | 0 | 0 | NA | NA |
| nsp16-P28838 | nsp16 | LAP3 | 0 | 0.9888 | 0.93521568 | 0 | 1 | 1 | NA | 0 | 0 |
| nsp16-P43686 | nsp16 | PSMC4 | 0.75749 | 0 | 0 | 0.98 | 0 | 0 | 0 | NA | NA |
| nsp16-P51530 | nsp16 | DNA2 | 0 | 0.79299 | 0.93085338 | 0 | 0.33 | 1 | NA | 0.2 | 0 |
| nsp16-P51659 | nsp16 | HSD17B4 | 0.82439 | 0 | 0.310191794 | 0.98 | 0 | 0.31 | 0 | NA | 0.32 |
| nsp16-P54802 | nsp16 | NAGLU | 0.98997 | 0 | 0 | 1 | 0 | 0 | 0 | NA | NA |
| nsp16-Q05086 | nsp16 | UBE3A | 0 | 0 | 0.993205727 | 0 | 0 | 1 | NA | NA | 0 |
| nsp16-Q12923 | nsp16 | PTPN13 | 0 | 0.035145 | 0.82472846 | 0 | 0 | 1 | NA | 0.74 | 0 |
| nsp16-Q13043 | nsp16 | STK4 | 0 | 0 | 0.936895908 | 0 | 0 | 1 | NA | NA | 0 |
| nsp16-Q13049 | nsp16 | TRIM32 | 0 | 0 | 0.988853916 | 0 | 0 | 1 | NA | NA | 0 |
| nsp16-Q13188 | nsp16 | STK3 | 0.68551 | 0 | 0.816118789 | 0 | 0 | 1 | 0.75 | NA | 0 |
| nsp16-Q13438 | nsp16 | OS9 | 0.99193 | 0 | 0.059439168 | 1 | 0 | 0 | 0 | NA | 0.72 |
| nsp16-Q15345 | nsp16 | LRRC41 | 0 | 0 | 0.988401417 | 0 | 0 | 0.97 | NA | NA | 0.01 |
| nsp16-Q15796 | nsp16 | SMAD2 | 0.96209 | 0 | 0 | 1 | 0 | 0 | 0 | NA | NA |
| nsp16-Q53EZ4 | nsp16 | CEP55 | 0 | 0 | 0.712072426 | 0 | 0 | 1 | NA | NA | 0 |
| nsp16-Q567U6 | nsp16 | CCDC93 | 0 | 0.80434 | 0.99302779 | 0 | 0.97 | 1 | NA | 0.01 | 0 |
| nsp16-Q5SVZ6 | nsp16 | ZMYM1 | 0 | 0.9891 | 0.994026056 | 0 | 1 | 1 | NA | 0 | 0 |
| nsp16-Q5SZL2 | nsp16 | CEP85L | 0 | 0.6041 | 0.993496095 | 0 | 0 | 1 | NA | 0.74 | 0 |
| nsp16-Q5VUJ6 | nsp16 | LRCH2 | 0 | 0 | 0.962503191 | 0 | 0 | 1 | NA | NA | 0 |
| nsp16-Q63ZY3 | nsp16 | KANK2 | 0 | 0 | 0.991823966 | 0 | 0 | 1 | NA | NA | 0 |
| nsp16-Q6GYQ0 | nsp16 | RALGAPA1 | 0 | 0 | 0.977416641 | 0 | 0 | 0.98 | NA | NA | 0 |
| nsp16-Q6IEG0 | nsp16 | SNRNP48 | 0 | 0 | 0.787090668 | 0 | 0 | 0.99 | NA | NA | 0 |
| nsp16-Q6PJI9 | nsp16 | WDR59 | 0.91343 | 0 | 0 | 0.95 | 0 | 0 | 0.01 | NA | NA |
| nsp16-Q6ZU80 | nsp16 | CEP128 | 0 | 0 | 0.893091909 | 0 | 0 | 1 | NA | NA | 0 |
| nsp16-Q6ZWJ1 | nsp16 | STXBP4 | 0 | 0 | 0.985046716 | 0 | 0 | 0.98 | NA | NA | 0 |
| nsp16-Q70EL1 | nsp16 | USP54 | 0 | 0 | 0.718980196 | 0 | 0 | 1 | NA | NA | 0 |
| nsp16-Q7Z3J2 | nsp16 | VPS35L | 0 | 0.68551 | 0.99120106 | 0 | 0 | 0.99 | NA | 0.74 | 0 |
| nsp16-Q7Z4G1 | nsp16 | COMMD6 | 0 | 0 | 0.993976899 | 0 | 0 | 0.95 | NA | NA | 0.01 |
| nsp16-Q86SQ0 | nsp16 | PHLDB2 | 0 | 0 | 0.831826435 | 0 | 0 | 1 | NA | NA | 0 |
| nsp16-Q86W92 | nsp16 | PPFIBP1 | 0 | 0 | 0.968360808 | 0 | 0 | 1 | NA | NA | 0 |
| nsp16-Q86X10 | nsp16 | RALGAPB | 0 | 0 | 0.983214673 | 0 | 0 | 1 | NA | NA | 0 |
| nsp16-Q8IUD2 | nsp16 | ERC1 | 0 | 0.9266 | 0.921350502 | 0 | 1 | 1 | NA | 0 | 0 |
| nsp16-Q8IWR1 | nsp16 | TRIM59 | 0.95769 | 0 | 0 | 0.66 | 0 | 0 | 0.05 | NA | NA |
| nsp16-Q8N668 | nsp16 | COMMD1 | 0 | 0 | 0.961313726 | 0 | 0 | 0.66 | NA | NA | 0.05 |
| nsp16-Q8TEM1 | nsp16 | NUP210 | 0 | 0.98108 | 0.850755735 | 0 | 1 | 1 | NA | 0 | 0 |
| nsp16-Q92995 | nsp16 | USP13 | 0.98234 | 0 | 0 | 1 | 0 | 0 | 0 | NA | NA |
| nsp16-Q96DZ1 | nsp16 | ERLEC1 | 0.78671 | 0 | 0.384798111 | 1 | 0 | 0.97 | 0 | NA | 0.01 |
| nsp16-Q96HP0 | nsp16 | DOCK6 | 0 | 0 | 0.990342796 | 0 | 0 | 1 | NA | NA | 0 |
| nsp16-Q96II8 | nsp16 | LRCH3 | 0 | 0 | 0.93763489 | 0 | 0 | 1 | NA | NA | 0 |
| nsp16-Q96IV0 | nsp16 | NGLY1 | 0.96057 | 0 | 0 | 1 | 0 | 0 | 0 | NA | NA |
| nsp16-Q96RU2 | nsp16 | USP28 | 0.97728 | 0 | 0 | 0.97 | 0 | 0 | 0 | NA | NA |
| nsp16-Q9BVQ7 | nsp16 | SPATA5L1 | 0 | 0 | 0.98126167 | 0 | 0 | 1 | NA | NA | 0 |
| nsp16-Q9GZQ3 | nsp16 | COMMD5 | 0 | 0 | 0.992994501 | 0 | 0 | 1 | NA | NA | 0 |
| nsp16-Q9H000 | nsp16 | MKRN2 | 0 | 0 | 0.71582382 | 0 | 0 | 1 | NA | NA | 0 |
| nsp16-Q9H0H0 | nsp16 | INTS2 | 0 | 0.31941 | 0.938340768 | 0 | 0.32 | 1 | NA | 0.28 | 0 |
| nsp16-Q9H4B6 | nsp16 | SAV1 | 0 | 0 | 0.869610136 | 0 | 0 | 1 | NA | NA | 0 |
| nsp16-Q9NVH2 | nsp16 | INTS7 | 0 | 0 | 0.92002501 | 0 | 0 | 1 | NA | NA | 0 |
| nsp16-Q9NX08 | nsp16 | COMMD8 | 0 | 0 | 0.936985686 | 0 | 0 | 0.89 | NA | NA | 0.01 |
| nsp16-Q9P000 | nsp16 | COMMD9 | 0 | 0 | 0.983665198 | 0 | 0 | 0.99 | NA | NA | 0 |
| nsp16-Q9P209 | nsp16 | CEP72 | 0.96027 | 0 | 0.685510246 | 1 | 0 | 0 | 0 | NA | 0.72 |
| nsp16-Q9P2D0 | nsp16 | IBTK | 0 | 0 | 0.774163503 | 0 | 0 | 1 | NA | NA | 0 |
| nsp16-Q9P2S5 | nsp16 | WRAP73 | 0 | 0 | 0.98754455 | 0 | 0 | 1 | NA | NA | 0 |
| nsp16-Q9UBI1 | nsp16 | COMMD3 | 0 | 0 | 0.989352281 | 0 | 0 | 1 | NA | NA | 0 |
| nsp16-Q9UHD2 | nsp16 | TBK1 | 0 | 0 | 0.730696528 | 0 | 0 | 1 | NA | NA | 0 |
| nsp16-Q9UHP3 | nsp16 | USP25 | 0 | 0 | 0.980380642 | 0 | 0 | 1 | NA | NA | 0 |
| nsp16-Q9UKF6 | nsp16 | CPSF3 | 0 | 0.89275 | 0.731969888 | 0 | 1 | 1 | NA | 0 | 0 |
| nsp16-Q9ULA0 | nsp16 | DNPEP | 0.92879 | 0 | 0 | 1 | 0 | 0 | 0 | NA | NA |
| nsp16-Q9UN81 | nsp16 | L1RE1 | 0 | 0 | 0.871349588 | 0 | 0 | 0.97 | NA | NA | 0.01 |
| nsp16-Q9Y2D8 | nsp16 | SSX2IP | 0 | 0.99395 | 0.944408372 | 0 | 0 | 1 | NA | 0.74 | 0 |
| nsp16-Q9Y2K2 | nsp16 | SIK3 | 0 | 0 | 0.977256516 | 0 | 0 | 1 | NA | NA | 0 |
| nsp16-Q9Y2S7 | nsp16 | POLDIP2 | 0.22683 | 0.7418 | 0.186930874 | 0 | 1 | 0.32 | 0.75 | 0 | 0.24 |
| nsp16-Q9Y305 | nsp16 | ACOT9 | 0.95763 | 0 | 0 | 1 | 0 | 0 | 0 | NA | NA |
| nsp16-Q9Y6G5 | nsp16 | COMMD10 | 0 | 0 | 0.992408318 | 0 | 0 | 1 | NA | NA | 0 |
| nsp2-O00186 | nsp2 | STXBP3 | 0.99168 | 0 | 0 | 1 | 0 | 0 | 0 | NA | NA |
| nsp2-O00303 | nsp2 | EIF3F | 0.53431 | 0.87273 | 0 | 1 | 1 | 0 | 0 | 0 | NA |
| nsp2-O00746 | nsp2 | NME4 | 0.80747 | 0.39111 | 0 | 0.95 | 0.32 | 0 | 0.01 | 0.28 | NA |
| nsp2-O14975 | nsp2 | SLC27A2 | 0.46144 | 0.42751 | 0.915803486 | 0.64 | 0.65 | 0.99 | 0.08 | 0.07 | 0 |
| nsp2-O15372 | nsp2 | EIF3H | 0.46627 | 0.71459 | 0.019650551 | 1 | 1 | 0 | 0 | 0 | 0.69 |
| nsp2-O60573 | nsp2 | EIF4E2 | 0.51532 | 0.83022 | 0.806833749 | 1 | 1 | 1 | 0 | 0 | 0 |
| nsp2-O75821 | nsp2 | EIF3G | 0.34433 | 0.76953 | 0 | 1 | 1 | 0 | 0 | 0 | NA |
| nsp2-O75822 | nsp2 | EIF3J | 0.56841 | 0.85594 | 0 | 0.99 | 1 | 0 | 0 | 0 | NA |
| nsp2-P00387 | nsp2 | CYB5R3 | 0.73714 | 0.2649 | 0 | 1 | 0 | 0 | 0 | 0.74 | NA |
| nsp2-P15954 | nsp2 | COX7C | 0.9895 | 0 | 0.442430132 | 0.97 | 0 | 0 | 0.01 | NA | 0.69 |
| nsp2-P16435 | nsp2 | POR | 0.74761 | 0.45328 | 0.710961769 | 1 | 0.66 | 1 | 0 | 0.07 | 0 |
| nsp2-P52306 | nsp2 | RAP1GDS1 | 0 | 0.92777 | 0.991635744 | 0 | 1 | 1 | NA | 0 | 0 |
| nsp2-P60228 | nsp2 | EIF3E | 0.54907 | 0.75501 | 0 | 1 | 1 | 0 | 0 | 0 | NA |
| nsp2-Q10471 | nsp2 | GALNT2 | 0.98389 | 0 | 0 | 0.97 | 0 | 0 | 0 | NA | NA |
| nsp2-Q13423 | nsp2 | NNT | 0.77519 | 0 | 0 | 0.97 | 0 | 0 | 0 | NA | NA |
| nsp2-Q14152 | nsp2 | EIF3A | 0.52249 | 0.86374 | 0 | 1 | 1 | 0 | 0 | 0 | NA |
| nsp2-Q15650 | nsp2 | TRIP4 | 0.87852 | 0 | 0 | 1 | 0 | 0 | 0 | NA | NA |
| nsp2-Q2M389 | nsp2 | WASHC4 | 0 | 0 | 0.972115182 | 0 | 0 | 0.99 | NA | NA | 0 |
| nsp2-Q5SZL2 | nsp2 | CEP85L | 0.86472 | 0 | 0 | 0.67 | 0 | 0 | 0.04 | NA | NA |
| nsp2-Q5T1M5 | nsp2 | FKBP15 | 0 | 0.97855 | 0.988056696 | 0 | 0.63 | 1 | NA | 0.1 | 0 |
| nsp2-Q5VT66 | nsp2 | MARC1 | 0.83301 | 0 | 0 | 0.99 | 0 | 0 | 0 | NA | NA |
| nsp2-Q6NUN9 | nsp2 | ZNF746 | 0.96549 | 0.85087 | 0 | 1 | 0.66 | 0 | 0 | 0.05 | NA |
| nsp2-Q6Y7W6 | nsp2 | GIGYF2 | 0.76827 | 0.87377 | 0.767224555 | 1 | 1 | 1 | 0 | 0 | 0 |
| nsp2-Q7L2H7 | nsp2 | EIF3M | 0.62747 | 0.96342 | 0 | 1 | 1 | 0 | 0 | 0 | NA |
| nsp2-Q86UK7 | nsp2 | ZNF598 | 0.48357 | 0.76844 | 0.56549083 | 1 | 1 | 1 | 0 | 0 | 0 |
| nsp2-Q8N3C0 | nsp2 | ASCC3 | 0.83183 | 0 | 0 | 1 | 0 | 0 | 0 | NA | NA |
| nsp2-Q8N9N2 | nsp2 | ASCC1 | 0.98223 | 0 | 0 | 1 | 0 | 0 | 0 | NA | NA |
| nsp2-Q8NBU5 | nsp2 | ATAD1 | 0.72843 | 0 | 0 | 1 | 0 | 0 | 0 | NA | NA |
| nsp2-Q8TF46 | nsp2 | DIS3L | 0.99038 | 0 | 0 | 1 | 0 | 0 | 0 | NA | NA |
| nsp2-Q8WVC6 | nsp2 | DCAKD | 0.77573 | 0 | 0 | 0.97 | 0 | 0 | 0.01 | NA | NA |
| nsp2-Q96A26 | nsp2 | FAM162A | 0.79955 | 0.014345 | 0.011155417 | 0.98 | 0 | 0 | 0 | 0.74 | 0.69 |
| nsp2-Q96B26 | nsp2 | EXOSC8 | 0.79211 | 0 | 0 | 0.66 | 0 | 0 | 0.05 | NA | NA |
| nsp2-Q96D09 | nsp2 | GPRASP2 | 0.98996 | 0 | 0 | 1 | 0 | 0 | 0 | NA | NA |
| nsp2-Q99613 | nsp2 | EIF3C | 0.9926 | 0.99317 | 0 | 1 | 1 | 0 | 0 | 0 | NA |
| nsp2-Q9BQ70 | nsp2 | TCF25 | 0.82229 | 0 | 0 | 1 | 0 | 0 | 0 | NA | NA |
| nsp2-Q9C037 | nsp2 | TRIM4 | 0.35683 | 0.76789 | 0 | 0 | 0.98 | 0 | 0.75 | 0 | NA |
| nsp2-Q9H1I8 | nsp2 | ASCC2 | 0.88018 | 0 | 0 | 1 | 0 | 0 | 0 | NA | NA |
| nsp2-Q9HD20 | nsp2 | ATP13A1 | 0.93754 | 0 | 0 | 0.98 | 0 | 0 | 0 | NA | NA |
| nsp2-Q9UBQ5 | nsp2 | EIF3K | 0.54617 | 0.73776 | 0 | 1 | 1 | 0 | 0 | 0 | NA |
| nsp2-Q9UH62 | nsp2 | ARMCX3 | 0.98889 | 0 | 0 | 0.95 | 0 | 0 | 0.01 | NA | NA |
| nsp2-Q9UPQ9 | nsp2 | TNRC6B | 0.73711 | 0 | 0 | 1 | 0 | 0 | 0 | NA | NA |
| nsp2-Q9Y262 | nsp2 | EIF3L | 0.46611 | 0.87362 | 0 | 1 | 1 | 0 | 0 | 0 | NA |
| nsp4-P13674 | nsp4 | P4HA1 | 0.90323 | 0 | 0.364154115 | 1 | 0 | 0.33 | 0 | NA | 0.19 |
| nsp4-P14735 | nsp4 | IDE | 0 | 0.98862 | 0.918031442 | 0 | 1 | 1 | NA | 0 | 0 |
| nsp4-P49257 | nsp4 | LMAN1 | 0.76853 | 0.57914 | 0 | 1 | 0 | 0 | 0 | 0.74 | NA |
| nsp4-P62072 | nsp4 | TIMM10 | 0 | 0.043526 | 0.961471982 | 0 | 0 | 1 | NA | 0.74 | 0 |
| nsp4-P62699 | nsp4 | YPEL5 | 0 | 0.99361 | 0 | 0 | 0.99 | 0 | NA | 0 | NA |
| nsp4-Q13586 | nsp4 | STIM1 | 0.97869 | 0 | 0 | 0.96 | 0 | 0 | 0.01 | NA | NA |
| nsp4-Q2TAA5 | nsp4 | ALG11 | 0 | 0.60123 | 0.72745605 | 0 | 1 | 1 | NA | 0 | 0 |
| nsp4-Q6VN20 | nsp4 | RANBP10 | 0 | 0.99277 | 0 | 0 | 1 | 0 | NA | 0 | NA |
| nsp4-Q7L5Y9 | nsp4 | MAEA | 0 | 0.98917 | 0 | 0 | 0.98 | 0 | NA | 0 | NA |
| nsp4-Q8NBJ7 | nsp4 | SUMF2 | 0.99115 | 0 | 0 | 0.99 | 0 | 0 | 0 | NA | NA |
| nsp4-Q8NFQ8 | nsp4 | TOR1AIP2 | 0.7969 | 0 | 0 | 1 | 0 | 0 | 0 | NA | NA |
| nsp4-Q8TEM1 | nsp4 | NUP210 | 0.39242 | 0.0039899 | 0.710174697 | 1 | 0 | 1 | 0 | 0.74 | 0 |
| nsp4-Q92643 | nsp4 | PIGK | 0.82887 | 0.22696 | 0.421421444 | 1 | 0 | 0.66 | 0 | 0.74 | 0.03 |
| nsp4-Q969N2 | nsp4 | PIGT | 0.70908 | 0 | 0.353983625 | 1 | 0 | 0.33 | 0 | NA | 0.19 |
| nsp4-Q96S59 | nsp4 | RANBP9 | 0 | 0.9935 | 0 | 0 | 1 | 0 | NA | 0 | NA |
| nsp4-Q9BSF4 | nsp4 | TIMM29 | 0 | 0 | 0.986980311 | 0 | 0 | 1 | NA | NA | 0 |
| nsp4-Q9H7D7 | nsp4 | WDR26 | 0 | 0.92941 | 0 | 0 | 1 | 0 | NA | 0 | NA |
| nsp4-Q9H871 | nsp4 | RMND5A | 0 | 0.9774 | 0 | 0 | 0.98 | 0 | NA | 0 | NA |
| nsp4-Q9NVH1 | nsp4 | DNAJC11 | 0 | 0 | 0.726866873 | 0 | 0 | 1 | NA | NA | 0 |
| nsp4-Q9NWU2 | nsp4 | GID8 | 0 | 0.98069 | 0 | 0 | 1 | 0 | NA | 0 | NA |
| nsp4-Q9Y5J6 | nsp4 | TIMM10B | 0 | 0 | 0.985104055 | 0 | 0 | 0.98 | NA | NA | 0 |
| nsp4-Q9Y5J7 | nsp4 | TIMM9 | 0 | 0 | 0.913806284 | 0 | 0 | 1 | NA | NA | 0 |
| nsp6-O75964 | nsp6 | ATP5MG | 0.021184 | 0.42343 | 0.717265558 | 0 | 1 | 1 | 0.75 | 0 | 0 |
| nsp6-P25685 | nsp6 | DNAJB1 | 0.83377 | 0 | 0 | 0.99 | 0 | 0 | 0 | NA | NA |
| nsp6-Q15904 | nsp6 | ATP6AP1 | 0.41324 | 0 | 0.989106922 | 0.62 | 0 | 1 | 0.09 | NA | 0 |
| nsp6-Q99720 | nsp6 | SIGMAR1 | 0 | 0.74095 | 0.842213253 | 0 | 1 | 1 | NA | 0 | 0 |
| nsp6-Q9H7F0 | nsp6 | ATP13A3 | 0 | 0.27018 | 0.805525853 | 0 | 0 | 1 | NA | 0.74 | 0 |
| nsp6-Q9UDY4 | nsp6 | DNAJB4 | 0.87935 | 0 | 0 | 0.66 | 0 | 0 | 0.05 | NA | NA |
| nsp7-A8MTT3 | nsp7 | CEBPZOS | 0.99309 | 0.98607 | 0.988878577 | 1 | 0.98 | 0.64 | 0 | 0 | 0.08 |
| nsp7-O00116 | nsp7 | AGPS | 0.63068 | 0.6251 | 0.826490325 | 0.53 | 1 | 1 | 0.13 | 0 | 0 |
| nsp7-O14975 | nsp7 | SLC27A2 | 0.79874 | 0.28335 | 0.049938217 | 1 | 0.32 | 0 | 0 | 0.28 | 0.69 |
| nsp7-O43169 | nsp7 | CYB5B | 0.6157 | 0.41671 | 0.80351019 | 0.31 | 0.98 | 0.99 | 0.22 | 0 | 0 |
| nsp7-O94766 | nsp7 | B3GAT3 | 0.8801 | 0.74743 | 0.585758918 | 0.67 | 0.66 | 0.97 | 0.04 | 0.05 | 0 |
| nsp7-O95159 | nsp7 | ZFPL1 | 0.72814 | 0.089899 | 0 | 0.95 | 0.33 | 0 | 0.01 | 0.24 | NA |
| nsp7-O95573 | nsp7 | ACSL3 | 0.91283 | 0.61136 | 0.897068932 | 1 | 1 | 1 | 0 | 0 | 0 |
| nsp7-P00387 | nsp7 | CYB5R3 | 0.078917 | 0.75124 | 0.956349351 | 0 | 1 | 1 | 0.75 | 0 | 0 |
| nsp7-P11233 | nsp7 | RALA | 0.57983 | 0.35486 | 0.750366485 | 0.66 | 0.99 | 0.97 | 0.06 | 0 | 0 |
| nsp7-P21964 | nsp7 | COMT | 0.57953 | 0.39728 | 0.745231765 | 0.94 | 1 | 0.66 | 0.01 | 0 | 0.04 |
| nsp7-P51148 | nsp7 | RAB5C | 0 | 0.54146 | 0.87908593 | 0 | 1 | 1 | NA | 0 | 0 |
| nsp7-P51149 | nsp7 | RAB7A | 0 | 0.48171 | 0.972724229 | 0 | 1 | 1 | NA | 0 | 0 |
| nsp7-P61006 | nsp7 | RAB8A | 0.094078 | 0.75447 | 0.895744596 | 0 | 1 | 0.65 | 0.75 | 0 | 0.05 |
| nsp7-P61019 | nsp7 | RAB2A | 0 | 0.55131 | 0.97919572 | 0 | 0.99 | 0.65 | NA | 0 | 0.05 |
| nsp7-P61026 | nsp7 | RAB10 | 0.11387 | 0.40774 | 0.981443071 | 0 | 0.97 | 0.98 | 0.75 | 0.01 | 0 |
| nsp7-P61106 | nsp7 | RAB14 | 0.38785 | 0.36825 | 0.750712826 | 0.31 | 1 | 1 | 0.22 | 0 | 0 |
| nsp7-P61586 | nsp7 | RHOA | 0 | 0.37112 | 0.829029399 | 0 | 0.98 | 0.65 | NA | 0 | 0.05 |
| nsp7-P62820 | nsp7 | RAB1A | 0 | 0.43828 | 0.935289593 | 0 | 1 | 0.99 | NA | 0 | 0 |
| nsp7-P62873 | nsp7 | GNB1 | 0.027515 | 0.27496 | 0.839532136 | 0 | 0.33 | 0.98 | 0.75 | 0.24 | 0 |
| nsp7-P63218 | nsp7 | GNG5 | 0.32569 | 0.31298 | 0.817631566 | 0 | 0.63 | 0.65 | 0.75 | 0.1 | 0.05 |
| nsp7-Q12907 | nsp7 | LMAN2 | 0 | 0.74257 | 0.725773983 | 0 | 1 | 1 | NA | 0 | 0 |
| nsp7-Q13724 | nsp7 | MOGS | 0.80868 | 0.66843 | 0.782330987 | 1 | 1 | 1 | 0 | 0 | 0 |
| nsp7-Q2TAA5 | nsp7 | ALG11 | 0 | 0.9002 | 0.465050352 | 0 | 1 | 0.65 | NA | 0 | 0.05 |
| nsp7-Q53H12 | nsp7 | AGK | 0.70589 | 0.40457 | 0.581229943 | 1 | 1 | 1 | 0 | 0 | 0 |
| nsp7-Q5JTV8 | nsp7 | TOR1AIP1 | 0.037862 | 0.53637 | 0.74516805 | 0 | 0.95 | 0.65 | 0.75 | 0.01 | 0.05 |
| nsp7-Q5VT66 | nsp7 | MARC1 | 0.52585 | 0.82997 | 0.939721024 | 0 | 1 | 1 | 0.75 | 0 | 0 |
| nsp7-Q6P1M0 | nsp7 | SLC27A4 | 0.91017 | 0 | 0 | 1 | 0 | 0 | 0 | NA | NA |
| nsp7-Q6P1Q0 | nsp7 | LETMD1 | 0.97824 | 0.79121 | 0.686459543 | 1 | 1 | 1 | 0 | 0 | 0 |
| nsp7-Q6ZRP7 | nsp7 | QSOX2 | 0.96617 | 0.98889 | 0.794325146 | 0.97 | 1 | 0.67 | 0 | 0 | 0.03 |
| nsp7-Q7LGA3 | nsp7 | HS2ST1 | 0.5733 | 0.80849 | 0.706466834 | 0 | 1 | 1 | 0.75 | 0 | 0 |
| nsp7-Q8IUR0 | nsp7 | TRAPPC5 | 0 | 0.90869 | 0.877498541 | 0 | 0.95 | 0 | NA | 0.01 | 0.69 |
| nsp7-Q8N183 | nsp7 | NDUFAF2 | 0 | 0.76562 | 0.981444858 | 0 | 0.63 | 0.98 | NA | 0.1 | 0 |
| nsp7-Q8N2K0 | nsp7 | ABHD12 | 0.77849 | 0.2418 | 0.393580798 | 1 | 0 | 0.32 | 0 | 0.74 | 0.23 |
| nsp7-Q8N9F7 | nsp7 | GDPD1 | 0.98701 | 0.87982 | 0 | 1 | 0 | 0 | 0 | 0.74 | NA |
| nsp7-Q8NBU5 | nsp7 | ATAD1 | 0.73826 | 0.59996 | 0.63242046 | 1 | 1 | 1 | 0 | 0 | 0 |
| nsp7-Q8NBX0 | nsp7 | SCCPDH | 0.96651 | 0.99217 | 0.978675119 | 0.66 | 1 | 0.97 | 0.06 | 0 | 0 |
| nsp7-Q8WTV0 | nsp7 | SCARB1 | 0 | 0.98016 | 0.854406247 | 0 | 0.98 | 0.66 | NA | 0 | 0.03 |
| nsp7-Q8WUY8 | nsp7 | NAT14 | 0.94047 | 0.77941 | 0.720285746 | 1 | 1 | 1 | 0 | 0 | 0 |
| nsp7-Q8WVC6 | nsp7 | DCAKD | 0.91629 | 0.6736 | 0.862452335 | 1 | 1 | 1 | 0 | 0 | 0 |
| nsp7-Q96A26 | nsp7 | FAM162A | 0.85168 | 0.87704 | 0.748773582 | 1 | 1 | 1 | 0 | 0 | 0 |
| nsp7-Q96DA6 | nsp7 | DNAJC19 | 0.78729 | 0.877 | 0.981450126 | 0.64 | 0.66 | 0.98 | 0.08 | 0.06 | 0 |
| nsp7-Q96ER9 | nsp7 | CCDC51 | 0 | 0.8562 | 0.685510484 | 0 | 0.98 | 0 | NA | 0 | 0.69 |
| nsp7-Q96KC8 | nsp7 | DNAJC1 | 0 | 0.97979 | 0 | 0 | 0.98 | 0 | NA | 0 | NA |
| nsp7-Q9BQE4 | nsp7 | SELENOS | 0.70106 | 0.72526 | 0.701764404 | 0.95 | 1 | 1 | 0.01 | 0 | 0 |
| nsp7-Q9H7Z7 | nsp7 | PTGES2 | 0.97653 | 0.86482 | 0.764538331 | 1 | 1 | 0.99 | 0 | 0 | 0 |
| nsp7-Q9NP72 | nsp7 | RAB18 | 0 | 0.42172 | 0.756605088 | 0 | 0.66 | 0.65 | NA | 0.06 | 0.05 |
| nsp7-Q9NX40 | nsp7 | OCIAD1 | 0.90909 | 0.59218 | 0.690748962 | 1 | 1 | 1 | 0 | 0 | 0 |
| nsp7-Q9NYP7 | nsp7 | ELOVL5 | 0 | 0.84898 | 0.685510854 | 0 | 0.97 | 0 | NA | 0.01 | 0.69 |
| nsp7-Q9Y3D7 | nsp7 | PAM16 | 0.59373 | 0.9496 | 0.766727199 | 0 | 0.67 | 0.33 | 0.75 | 0.05 | 0.19 |
| nsp7-Q9Y5J7 | nsp7 | TIMM9 | 0.77215 | 0.3231 | 0.074367865 | 0.66 | 0 | 0 | 0.05 | 0.74 | 0.69 |
| nsp8-O00566 | nsp8 | MPHOSPH10 | 0.63142 | 0.79381 | 0.728559172 | 0.97 | 0.98 | 0.66 | 0 | 0 | 0.03 |
| nsp8-O15381 | nsp8 | NVL | 0.92746 | 0.36364 | 0 | 0.97 | 0.66 | 0 | 0 | 0.05 | NA |
| nsp8-O60287 | nsp8 | URB1 | 0.75107 | 0.62158 | 0.586595339 | 1 | 1 | 1 | 0 | 0 | 0 |
| nsp8-O76094 | nsp8 | SRP72 | 0.50317 | 0.72069 | 0.739540656 | 1 | 1 | 1 | 0 | 0 | 0 |
| nsp8-O95260 | nsp8 | ATE1 | 0 | 0.83722 | 0.804292637 | 0 | 1 | 1 | NA | 0 | 0 |
| nsp8-O95373 | nsp8 | IPO7 | 0.73192 | 0 | 0 | 1 | 0 | 0 | 0 | NA | NA |
| nsp8-O95707 | nsp8 | POP4 | 0.74158 | 0.86009 | 0.8670804 | 0.97 | 0.32 | 0.32 | 0.01 | 0.28 | 0.23 |
| nsp8-O96028 | nsp8 | NSD2 | 0.49946 | 0.97503 | 0.864651959 | 0 | 0.65 | 0.65 | 0.75 | 0.09 | 0.05 |
| nsp8-P09132 | nsp8 | SRP19 | 0.56792 | 0.85781 | 0.832502372 | 1 | 1 | 1 | 0 | 0 | 0 |
| nsp8-P10644 | nsp8 | PRKAR1A | 0.98253 | 0 | 0 | 0.99 | 0 | 0 | 0 | NA | NA |
| nsp8-P42285 | nsp8 | MTREX | 0.7549 | 0.50799 | 0.565305623 | 1 | 0.66 | 0.65 | 0 | 0.05 | 0.05 |
| nsp8-P51114 | nsp8 | FXR1 | 0.8556 | 0.3336 | 0.336477658 | 1 | 1 | 1 | 0 | 0 | 0 |
| nsp8-P51116 | nsp8 | FXR2 | 0.75416 | 0.35976 | 0.373677635 | 1 | 1 | 1 | 0 | 0 | 0 |
| nsp8-P61011 | nsp8 | SRP54 | 0.39521 | 0.6574 | 0.755584148 | 0.76 | 0.65 | 0.99 | 0.03 | 0.08 | 0 |
| nsp8-P82663 | nsp8 | MRPS25 | 0.60063 | 0.55893 | 0.826437119 | 0.95 | 0.32 | 1 | 0.01 | 0.28 | 0 |
| nsp8-Q03701 | nsp8 | CEBPZ | 0.7073 | 0.44586 | 0.52197305 | 1 | 1 | 1 | 0 | 0 | 0 |
| nsp8-Q12788 | nsp8 | TBL3 | 0.74964 | 0.46634 | 0.380828129 | 1 | 1 | 1 | 0 | 0 | 0 |
| nsp8-Q13206 | nsp8 | DDX10 | 0.75703 | 0.78016 | 0.755753594 | 1 | 1 | 1 | 0 | 0 | 0 |
| nsp8-Q14146 | nsp8 | URB2 | 0.88233 | 0.56549 | 0.336186744 | 1 | 0.99 | 0.33 | 0 | 0 | 0.18 |
| nsp8-Q14692 | nsp8 | BMS1 | 0.68604 | 0.7344 | 0.616523719 | 1 | 1 | 1 | 0 | 0 | 0 |
| nsp8-Q15269 | nsp8 | PWP2 | 0.77802 | 0.39761 | 0.288654637 | 0.98 | 0.98 | 0.67 | 0 | 0 | 0.03 |
| nsp8-Q15397 | nsp8 | PUM3 | 0.6236 | 0.72164 | 0.626646614 | 1 | 1 | 1 | 0 | 0 | 0 |
| nsp8-Q16531 | nsp8 | DDB1 | 0.94832 | 0.29714 | 0.329839777 | 0.96 | 0.99 | 1 | 0.01 | 0 | 0 |
| nsp8-Q4GOJ3 | nsp8 | LARP7 | 0.43919 | 0.79384 | 0.812479682 | 1 | 1 | 1 | 0 | 0 | 0 |
| nsp8-Q76FK4 | nsp8 | NOL8 | 0.80515 | 0.63235 | 0.560442083 | 1 | 1 | 0.96 | 0 | 0 | 0.01 |
| nsp8-Q7L2J0 | nsp8 | MEPCE | 0.43695 | 0.78202 | 0.790978117 | 1 | 1 | 1 | 0 | 0 | 0 |
| nsp8-Q7Z4Q2 | nsp8 | HEATR3 | 0.98736 | 0 | 0 | 0.95 | 0 | 0 | 0.01 | NA | NA |
| nsp8-Q8IX01 | nsp8 | SUGP2 | 0.71554 | 0 | 0 | 0.95 | 0 | 0 | 0.01 | NA | NA |
| nsp8-Q8IY37 | nsp8 | DHX37 | 0.50147 | 0.98962 | 0 | 0.66 | 1 | 0 | 0.05 | 0 | NA |
| nsp8-Q8N5D0 | nsp8 | WDTC1 | 0.99156 | 0.015561 | 0.407783421 | 1 | 0 | 0.96 | 0 | 0.74 | 0.01 |
| nsp8-Q8N983 | nsp8 | MRPL43 | 0 | 0.99078 | 0 | 0 | 0.97 | 0 | NA | 0.01 | NA |
| nsp8-Q8NEJ9 | nsp8 | NGDN | 0.56745 | 0.64081 | 0.71407894 | 0.64 | 0.98 | 1 | 0.08 | 0 | 0 |
| nsp8-Q8NI36 | nsp | WDR36 | 0.77991 | 0.42551 | 0.47386872 | 0.98 | 1 | 1 | 0 | 0 | 0 |
| nsp8-Q8TC07 | nsp8 | TBC1D15 | 0.98574 | 0 | 0 | 1 | 0 | 0 | 0 | NA | NA |
| nsp8-Q96B26 | nsp8 | EXOSC8 | 0.5042 | 0.97866 | 0.990898225 | 0.64 | 0.98 | 1 | 0.08 | 0 | 0 |
| nsp8-Q96FK6 | nsp8 | WDR89 | 0.69287 | 0.99353 | 0 | 0.99 | 0.99 | 0 | 0 | 0 | NA |
| nsp8-Q96159 | nsp8 | NARS2 | 0.88015 | 0.067044 | 0.78185035 | 0.62 | 0 | 1 | 0.09 | 0.74 | 0 |
| nsp8-Q99547 | nsp8 | MPHOSPH6 | 0.75562 | 0.91098 | 0.974291683 | 0.94 | 0.33 | 0.32 | 0.01 | 0.21 | 0.23 |
| nsp8-Q9BSC4 | nsp8 | NOL10 | 0.90318 | 0.80021 | 0.807819511 | 1 | 1 | 1 | 0 | 0 | 0 |
| nsp8-Q9GZL7 | nsp8 | WDR12 | 0.83699 | 0.61793 | 0.562899877 | 1 | 0.97 | 0.65 | 0 | 0.01 | 0.05 |
| nsp8-Q9H6F5 | nsp8 | CCDC86 | 0.56342 | 0.97057 | 0.736803661 | 0.64 | 0.97 | 1 | 0.07 | 0 | 0 |
| nsp8-Q9H6R4 | nsp8 | NOL6 | 0.73249 | 0.3704 | 0.355297835 | 1 | 1 | 1 | 0 | 0 | 0 |
| nsp8-Q9HD40 | nsp8 | SEPSECS | 0.974 | 0.40352 | 0.809559247 | 0.31 | 0.32 | 1 | 0.22 | 0.28 | 0 |
| nsp8-Q9NQT4 | nsp8 | EXOSC5 | 0.59082 | 0.64069 | 0.704291901 | 0.95 | 0.99 | 0.99 | 0.01 | 0 | 0 |
| nsp8-Q9NQT5 | nsp8 | EXOSC3 | 0.5731 | 0.60253 | 0.774797319 | 0.95 | 0.98 | 1 | 0.01 | 0 | 0 |
| nsp8-Q9NTK5 | nsp8 | OLA1 | 0.89068 | 0.013447 | 0.451456849 | 0.67 | 0 | 0.99 | 0.04 | 0.74 | 0 |
| nsp8-Q9NY61 | nsp8 | AATF | 0.65603 | 0.85156 | 0.783703681 | 0.95 | 1 | 1 | 0.01 | 0 | 0 |
| nsp8-Q9UGI8 | nsp8 | TES | 0 | 0.99046 | 0.685510876 | 0 | 1 | 0.33 | NA | 0 | 0.19 |
| nsp8-Q9UHG3 | nsp8 | PCYOX1 | 0.99165 | 0 | 0 | 1 | 0 | 0 | 0 | NA | NA |
| nsp8-Q9UL40 | nsp8 | ZNF346 | 0.26738 | 0.7147 | 0 | 0.14 | 0.98 | 0 | 0.39 | 0 | NA |
| nsp8-Q9ULT8 | nsp8 | HECTD1 | 0 | 0.82709 | 0.885504785 | 0 | 1 | 1 | NA | 0 | 0 |
| nsp8-Q9ULX6 | nsp8 | AKAP8L | 0.81872 | 0 | 0.213643659 | 0.95 | 0 | 0.64 | 0.01 | NA | 0.08 |
| nsp8-Q9Y399 | nsp8 | MRPS2 | 0 | 0 | 0.972057569 | 0 | 0 | 0.65 | NA | NA | 0.05 |
| nsp8-Q9Y3A4 | nsp8 | RRP7A | 0.79389 | 0.33638 | 0.341118627 | 0.97 | 0 | 0.32 | 0 | 0.74 | 0.23 |
| nsp9-O00142 | nsp9 | TK2 | 0 | 0.98401 | 0.68551879 | 0 | 1 | 1 | NA | 0 | 0 |
| nsp9-O00233 | nsp9 | PSMD9 | 0.99068 | 0 | 0 | 0.97 | 0 | 0 | 0.01 | NA | NA |
| nsp9-P13984 | nsp9 | GTF2F2 | 0 | 0.59529 | 0.877426938 | 0 | 0.96 | 1 | NA | 0.01 | 0 |
| nsp9-P21281 | nsp9 | ATP6V1B2 | 0.96322 | 0 | 0 | 0.66 | 0 | 0 | 0.05 | NA | NA |
| nsp9-P35555 | nsp9 | FBN1 | 0 | 0.68551 | 0.992372395 | 0 | 0.32 | 1 | NA | 0.28 | 0 |
| nsp9-P35556 | nsp9 | FBN2 | 0 | 0.99111 | 0.991012329 | 0 | 1 | 1 | NA | 0 | 0 |
| nsp9-P35658 | nsp9 | NUP214 | 0.031562 | 0 | 0.962233264 | 0 | 0 | 1 | 0.75 | NA | 0 |
| nsp9-P37198 | nsp9 | NUP62 | 0 | 0.16429 | 0.993010451 | 0 | 0 | 1 | NA | 0.74 | 0 |
| nsp9-P38606 | nsp9 | ATP6V1A | 0.97813 | 0 | 0 | 1 | 0 | 0 | 0 | NA | NA |
| nsp9-P41250 | nsp9 | GARS | 0.91459 | 0 | 0 | 0.94 | 0 | 0 | 0.01 | NA | NA |
| nsp9-P49419 | nsp9 | ALDH7A1 | 0.89105 | 0 | 0 | 1 | 0 | 0 | 0 | NA | NA |
| nsp9-P61962 | nsp9 | DCAF7 | 0 | 0.76041 | 0.969234024 | 0 | 1 | 1 | NA | 0 | 0 |
| nsp9-P62310 | nsp9 | LSM3 | 0.87637 | 0 | 0 | 0.96 | 0 | 0 | 0.01 | NA | NA |
| nsp9-Q14232 | nsp9 | EIF2B1 | 0 | 0.77978 | 0.992001364 | 0 | 0.98 | 0 | NA | 0 | 0.69 |
| nsp9-Q15056 | nsp9 | EIF4H | 0 | 0.32352 | 0.86901939 | 0 | 0 | 1 | NA | 0.74 | 0 |
| nsp9-Q5SW79 | nsp9 | CEP170 | 0.88196 | 0 | 0 | 1 | 0 | 0 | 0 | NA | NA |
| nsp9-Q6SZW1 | nsp9 | SARM1 | 0.82032 | 0 | 0 | 0.66 | 0 | 0 | 0.05 | NA | NA |
| nsp9-Q7Z3B4 | nsp9 | NUP54 | 0 | 0 | 0.991624822 | 0 | 0 | 1 | NA | NA | 0 |
| nsp9-Q86YT6 | nsp9 | MIB1 | 0.9611 | 0.71417 | 0.89782233 | 1 | 1 | 1 | 0 | 0 | 0 |
| nsp9-Q8IWP9 | nsp9 | CCDC28A | 0.92122 | 0.089793 | 0 | 1 | 0.32 | 0 | 0 | 0.28 | NA |
| nsp9-Q8N0X7 | nsp9 | SPART | 0 | 0.83931 | 0.962964129 | 0 | 1 | 1 | NA | 0 | 0 |
| nsp9-Q8N1G2 | nsp9 | CMTR1 | 0 | 0.70971 | 0 | 0 | 0.67 | 0 | NA | 0.05 | NA |
| nsp9-Q8TD19 | nsp9 | NEK9 | 0.82535 | 0.77502 | 0.991972865 | 0.57 | 1 | 1 | 0.12 | 0 | 0 |
| nsp9-Q96F45 | nsp9 | ZNF503 | 0.078984 | 0.5176 | 0.777581447 | 0 | 1 | 1 | 0.75 | 0 | 0 |
| nsp9-Q96PM5 | nsp9 | RCHY1 | 0.80642 | 0 | 0 | 1 | 0 | 0 | 0 | NA | NA |
| nsp9-Q99567 | nsp9 | NUP88 | 0 | 0 | 0.92724312 | 0 | 0 | 0.99 | NA | NA | 0 |
| nsp9-Q9BU61 | nsp9 | NDUFAF3 | 0.89629 | 0 | 0 | 0.95 | 0 | 0 | 0.01 | NA | NA |
| nsp9-Q9BVL2 | nsp9 | NUP58 | 0 | 0 | 0.979586223 | 0 | 0 | 1 | NA | NA | 0 |
| nsp9-Q9NZL9 | nsp9 | MAT2B | 0 | 0 | 0.978282655 | 0 | 0 | 1 | NA | NA | 0 |
| nsp9-Q9UBX5 | nsp9 | FBLN5 | 0.99375 | 0 | 0.992002193 | 0 | 0 | 0.96 | 0.75 | NA | 0.01 |

| | FoldChange_{—} | FoldChange_{—} | FoldChange_{—} | K_Interaction | K_Interaction | K_Interaction | | Cluster_{—} | DIS_SARS1_{—} | DIS_SARS2_{—} | DIS_SARS2_{—} | DIS_SARS_{—} |
|---|---|---|---|---|---|---|---|---|---|---|---|---|
| Bait_Prey | MERS | SARS1 | SARS2 | Score_MERS | Score_SARS1 | Score_SARS2 | Cluster | Assignments | MERS | MERS | SARS1 | MERS |
| E-O00203 | 1.6 | 16.67 | 46.67 | 0.1349 | 0.618285 | 0.976775048 | 4 | S2_S1 | 0.483385 | 0.841875048 | 0.358490048 | 0.662630024 |
| E-O15270 | 30 | 0 | 0 | 0.932615 | 0 | 0 | 5 | M | −0.932615 | −0.932615 | NA | −0.932615 |
| E-O43505 | 40 | 0 | 0 | 0.85674 | 0 | 0 | 5 | M | −0.85674 | −0.85674 | NA | −0.85674 |
| E-O60885 | 1 | 3.33 | 26.67 | 0.0475195 | 0.342755 | 0.974244175 | 6 | S2 | NA | 0.926724675 | 0.631489175 | NA |
| E-O75787 | 46.67 | 0 | 0 | 0.920175 | 0 | 0 | 5 | M | −0.920175 | −0.920175 | NA | −0.920175 |
| E-P01861 | 23.33 | 0 | 0 | 0.970695 | 0 | 0 | 5 | M | −0.970695 | −0.970695 | NA | −0.970695 |
| E-P25440 | 0 | 5.33 | 70 | 0 | 0.49844 | 0.953296438 | 4 | S2_S1 | 0.49844 | 0.953296438 | 0.454856438 | 0.725868219 |
| E-Q5T9L3 | 23.33 | 0 | 0 | 0.925655 | 0 | 0 | 5 | M | −0.925655 | −0.925655 | NA | −0.925655 |
| E-Q6DD88 | 116.67 | 0 | 0 | 0.991585 | 0 | 0 | 5 | M | −0.991585 | −0.991585 | NA | −0.991585 |
| E-Q6UX04 | 0.57 | 36.67 | 26.67 | 0.01946 | 0.816765 | 0.77655458 | 4 | S2_S1 | 0.797305 | 0.75709458 | −0.04021042 | 0.77719979 |
| E-Q86VM9 | 0 | 10 | 26.67 | 0 | 0.30879 | 0.88320752 | 6 | S2 | NA | 0.88320752 | 0.57441752 | NA |
| E-Q8IWA5 | 0 | 0 | 26.67 | 0 | 0 | 0.965171417 | 6 | S2 | NA | 0.965171417 | 0.965171417 | NA |
| E-Q8IZ52 | 26.67 | 0 | 0 | 0.88676 | 0 | 0 | 5 | M | −0.88676 | −0.88676 | NA | −0.88676 |
| E-Q8WVM8 | 23.33 | 6.67 | 0 | 0.835675 | 0.15317 | 0 | 5 | M | −0.682505 | −0.835675 | NA | −0.75909 |
| E-Q8WY22 | 56.67 | 0 | 0 | 0.99562 | 0 | 0 | 5 | M | −0.99562 | −0.99562 | NA | −0.99562 |
| E-Q92665 | 0 | 20 | 0 | 0 | 0.90848 | 0 | 3 | S1 | 0.90848 | NA | −0.90848 | NA |
| E-Q9BTV4 | 293.33 | 0 | 0 | 0.937635 | 0 | 0 | 5 | M | −0.937635 | −0.937635 | NA | −0.937635 |
| E-Q9NPI6 | 63.33 | 0 | 0 | 0.98987 | 0 | 0 | 5 | M | −0.98987 | −0.98987 | NA | −0.98987 |
| E-Q9UBS3 | 36.67 | 0 | 0 | 0.97643 | 0 | 0 | 5 | M | −0.97643 | −0.97643 | NA | −0.97643 |
| E-Q9ULP9 | 0 | 23.33 | 0 | 0 | 0.943255 | 0 | 3 | S1 | 0.943255 | NA | −0.943255 | NA |
| E-Q9Y5L0 | 33.33 | 0 | 0 | 0.949885 | 0 | 0 | 5 | M | −0.949885 | −0.949885 | NA | −0.949885 |
| M-O15321 | 0 | 43.33 | 36.67 | 0 | 0.995725 | 0.77627478 | 4 | S2_S1 | 0.995725 | 0.77627478 | −0.21945022 | 0.88599989 |
| M-O15397 | 13.33 | 116.67 | 30 | 0.570365 | 0.85349 | 0.781026241 | 2 | S2_S1_M | 0.283125 | 0.21066124141 | −0.072463759 | 0.246893121 |
| M-O15431 | 0 | 20 | 3.33 | 0 | 0.846785 | 0.34275538 | 3 | S1 | 0.846785 | NA | −0.504029621 | NA |
| M-O43156 | 0 | 23.33 | 0 | 0 | 0.978405 | 0 | 3 | S1 | 0.978405 | NA | −0.978405 | NA |
| M-O60779 | 0 | 23.33 | 13.33 | 0 | 0.979675 | 0.532466642 | 4 | S2_S1 | 0.979675 | 0.532466642 | −0.447208358 | 0.756070821 |
| M-O75027 | 0 | 70 | 23.33 | 0 | 0.86962 | 0.624016684 | 4 | S2_S1 | 0.86962 | 0.624016684 | 60.245603316 | 0.746818342 |
| M-O75439 | 0 | 0 | 96.67 | 0 | 0 | 0.992560099 | 6 | S2 | NA | 0.992560099 | 0.992560099 | NA |
| M-O94822 | 20 | 116.67 | 53.33 | 0.966835 | 0.964045 | 0.768655234 | 2 | S2_S1_M | −0.00279 | 0.198179766 | −0.195389766 | −0.100484883 |
| M-O94829 | 10 | 43.33 | 16.67 | 0.485275 | 0.996345 | 0.458440959 | 1 | S1_M | 0.51107 | 0.026834042 | −0.537904042 | NA |
| M-O95070 | 0 | 20 | 23.33 | 0 | 0.56593 | 0.913000418 | 4 | S2_S1 | 0.56593 | 0.913000418 | 0.347070418 | 0.739465209 |
| M-O95674 | 26.67 | 63.33 | 43.33 | 0.971215 | 0.92897 | 0.764617921 | 2 | S2_S1_M | −0.042245 | −0.206597079 | −0.164352079 | −0.12442104 |
| M-O95864 | 0 | 40 | 20 | 0 | 0.974855 | 0.618584079 | 4 | S2_S1 | 0.974855 | 0.618584079 | −0.356270922 | 0.796719539 |
| M-P05026 | 0 | 50 | 36.67 | 0 | 0.99697 | 0.908812801 | 4 | S2_S1 | 0.99697 | 0.908812801 | −0.0881572 | 0.9528914 |
| M-P07384 | 10 | 70 | 30 | 0.316425 | 0.91324 | 0.726561706 | 4 | S2_S1 | 0.596815 | 0.410136706 | −0.186678295 | 0.503475853 |
| M-P11310 | 0 | 13.33 | 26.67 | 0 | 0.463645 | 0.847174285 | 4 | S2_S1 | 0.463645 | 0.847174285 | 0.383529285 | 0.655409642 |
| M-P13804 | 0 | 53.33 | 23.33 | 0 | 0.73912 | 0.844199148 | 4 | S2_S1 | 0.73912 | 0.844199148 | 0.105079148 | 0.791659574 |
| M-P20020 | 10 | 136.67 | 73.33 | 0.584485 | 0.940885 | 0.834548065 | 2 | S2_S1_M | 0.3564 | 0.250063065 | −0.106336935 | 0.303231533 |
| M-P23634 | 0 | 40 | 10 | 0 | 0.80781 | 0.374613027 | 3 | S1 | 0.80781 | NA | −0.433196974 | NA |
| M-P24390 | 0 | 20 | 16.67 | 0 | 0.83647 | 0.547097311 | 4 | S2_S1 | 0.83647 | 0.547097311 | −0.289372689 | 0.691783656 |
| M-P27105 | 0 | 26.67 | 30 | 0 | 0.83667 | 0.866485886 | 4 | S2_S1 | 0.83667 | 0.866485886 | 0.029815886 | 0.851577943 |
| M-P33527 | 0 | 130 | 0 | 0 | 0.985205 | 0 | 3 | S1 | 0.985205 | NA | −0.985205 | NA |
| M-P35670 | 0 | 26.67 | 0 | 0 | 0.98529 | 0 | 3 | S1 | 0.98529 | NA | −0.98529 | NA |
| M-P38435 | 0 | 43.33 | 20 | 0 | 0.96677 | 0.874983499 | 4 | S2_S1 | 0.96677 | 0.874983499 | −0.091786501 | 0.92087675 |
| M-P38606 | 0 | 33.33 | 26.67 | 0 | 0.67157 | 0.722469247 | 4 | S2_S1 | 0.67157 | 0.722469247 | 0.050899247 | 0.697019623 |
| M-P40763 | 0 | 36.67 | 0 | 0 | 0.93212 | 0 | 3 | S1 | 0.93212 | NA | −0.93212 | NA |
| M-P43003 | 13.33 | 50 | 30 | 0.64209 | 0.937355 | 0.834104623 | 2 | S2_S1_M | 0.295265 | 0.192014623 | −0.103250377 | 0.243639812 |
| M-P48556 | 0 | 16.67 | 20 | 0 | 0.501555 | 0.76571239 | 4 | S2_S1 | 0.501555 | 0.76571239 | 0.26415739 | 0.633633695 |
| M-P49768 | 13.33 | 26.67 | 10 | 0.646215 | 0.87984 | 0.269036888 | 1 | S1_M | 0.233625 | −0.377178113 | −0.610803113 | NA |
| M-P56589 | 10 | 30 | 0 | 0.308185 | 0.88283 | 0 | 3 | S1 | 0.574645 | NA | −0.88283 | NA |
| M-P61803 | 0 | 33.33 | 13.33 | 0 | 0.953365 | 0.432426583 | 3 | S1 | 0.953365 | NA | −0.520938418 | NA |
| M-P98194 | 16.67 | 93.33 | 76.67 | 0.801395 | 0.98219 | 0.718556551 | 2 | S2_S1_M | 0.180795 | −0.08283845 | −0.26363345 | 0.048978275 |
| M-Q00765 | 0 | 20 | 106.67 | 0 | 0.318965 | 0.956544254 | 6 | S2 | NA | 0.956544254 | 0.637579254 | NA |
| M-Q10713 | 0 | 0 | 93.33 | 0 | 0 | 0.995529908 | 6 | S2 | NA | 0.995529908 | 0.995529908 | NA |
| M-Q13409 | 0 | 30 | 10 | 0 | 0.86679 | 0.507755377 | 4 | S2_S1 | 0.86679 | 0.507755377 | −0.359034623 | 0.687272689 |
| M-Q13433 | 6.67 | 33.33 | 16.67 | 0.376695 | 0.95636 | 0.763076712 | 4 | S2_S1 | 0.579665 | 0.386381712 | −0.193283289 | 0.483023356 |
| M-Q13505 | 0 | 40 | 16.67 | 0 | 0.8498 | 0.695219357 | 4 | S2_S1 | 0.8498 | 0.695219357 | −0.154580643 | 0.772509679 |
| M-Q14CZ7 | 0 | 20 | 6.67 | 0 | 0.97197 | 0.1515916 | 3 | S1 | 0.97197 | NA | −0.820378401 | NA |
| M-Q15043 | 3.33 | 80 | 50 | 0.09189 | 0.860435 | 0.768785611 | 4 | S2_S1 | 0.768545 | 0.676895611 | −0.091649380 | 0.722720306 |
| M-Q15386 | 0 | 56.67 | 13.33 | 0 | 0.68976 | 0.452961442 | 4 | S2_S1 | 0.68976 | 0.452961442 | −0.236798559 | 0.571360721 |
| M-Q4KMQ2 | 0 | 10 | 93.33 | 0 | 0.592015 | 0.99695221 | 4 | S2_S1 | 0.592015 | 0.99695221 | 0.40493721 | 0.794483605 |
| M-Q53R41 | 30 | 80 | 73.33 | 0.77918 | 0.9303 | 0.811478783 | 2 | S2_S1_M | 0.15112 | 0.032298783 | −0.118821217 | 0.091709392 |
| M-Q5BJH7 | 6.67 | 23.33 | 33.33 | 0.18561 | 0.979675 | 0.798974774 | 4 | S2_S1 | 0.794065 | 0.613364774 | −0.180700226 | 0.703714887 |
| M-Q5H8A4 | 3.33 | 40 | 23.33 | 0.068225 | 0.994685 | 0.764183669 | 4 | S2_S1 | 0.92646 | 0.695958669 | −0.230501332 | 0.811209334 |
| M-Q5JRX3 | 0 | 3.33 | 70 | 0 | 0.00055545 | 0.976154116 | 6 | S2 | NA | 0.976154116 | 0.975598666 | NA |
| M-Q5T1Q4 | 0 | 23.33 | 0 | 0 | 0.978405 | 0 | 3 | S1 | 0.978405 | NA | −0.978405 | NA |
| M-Q5T9L3 | 3.33 | 56.67 | 40 | 0.043137 | 0.99547 | 0.808491442 | 4 | S2_S1 | 0.952333 | 0.765354442 | −0.186978550 | 0.858843721 |
| M-Q68DH5 | 23.33 | 3.33 | 3.33 | 0.968465 | 0.342755 | 0.122471482 | 5 | M | −0.62571 | 0.845993519 | NA | 0.735851759 |
| M-Q6AI08 | 0 | 23.33 | 0 | 0 | 0.899215 | 0 | 3 | S1 | 0.899215 | NA | −0.899215 | NA |
| M-Q6P3X3 | 66.67 | 116.67 | 16.67 | 0.87311 | 0.860405 | 0.346146123 | 1 | S1_M | −0.012705 | −0.526963877 | −0.514258877 | NA |
| M-Q6PJG6 | 0 | 36.67 | 0 | 0 | 0.995565 | 0 | 3 | S1 | 0.995565 | NA | −0.995565 | NA |
| M-Q6PML9 | 0 | 23.33 | 20 | 0 | 0.565555 | 0.768161621 | 4 | S2_S1 | 0.565555 | 0.768161621 | 0.202606621 | 0.666858311 |
| M-Q7L8L6 | 0 | 123.33 | 73.33 | 0 | 0.855235 | 0.879182944 | 4 | S2_S1 | 0.855235 | 0.879182944 | 0.023947944 | 0.867208972 |
| M-Q7RTS9 | 0 | 23.33 | 0 | 0 | 0.979675 | 0 | 3 | S1 | 0.979675 | NA | −0.979675 | NA |
| M-Q7Z3U7 | 0 | 30 | 6.67 | 0 | 0.980735 | 0.502755088 | 4 | S2_S1 | 0.980735 | 0.502755088 | −0.477979913 | 0.741745044 |
| M-Q86UL3 | 6.67 | 70 | 20 | 0.30488 | 0.924775 | 0.722494785 | 4 | S2_S1 | 0.619895 | 0.417614785 | −0.202280215 | 0.518754893 |
| M-Q8N1F8 | 0 | 20 | 0 | 0 | 0.97197 | 0 | 3 | S1 | 0.97197 | NA | −0.97197 | NA |
| M-Q8N5G2 | 0 | 40 | 0 | 0 | 0.8028 | 0 | 3 | S1 | 0.8028 | NA | −0.8028 | NA |
| M-Q8NDZ4 | 93.33 | 0 | 0 | 0.87384 | 0 | 0 | 5 | M | −0.87384 | −0.87384 | NA | −0.87384 |
| M-Q8NEW0 | 20 | 30 | 46.67 | 0.611695 | 0.79608 | 0.883486219 | 2 | S2_S1_M | 0.184385 | 0.271791219 | 0.087406219 | 0.228088109 |
| M-Q8TBF5 | 0 | 33.33 | 13.33 | 0 | 0.990045 | 0.378661581 | 3 | S1 | 0.990045 | NA | −0.61138342 | NA |
| M-Q8TCJ2 | 0 | 73.33 | 3.33 | 0 | 0.995485 | 0.008895195 | 3 | S1 | 0.995485 | NA | −0.986589805 | NA |
| M-Q8TEM1 | 426.67 | 3.33 | 0 | 0.86292 | 0.014931 | 0 | 5 | M | −0.847989 | −0.86292 | NA | −0.8554545 |
| M-Q8WUD6 | 0 | 26.67 | 20 | 0 | 0.938925 | 0.642987005 | 4 | S2_S1 | 0.938925 | 0.642987005 | −0.295937996 | 0.790956002 |
| M-Q8WY22 | 0 | 46.67 | 46.67 | 0 | 0.91244 | 0.787073353 | 4 | S2_S1 | 0.91244 | 0.787073353 | −0.125366648 | 0.849756676 |
| M-Q92604 | 0 | 23.33 | 23.33 | 0 | 0.978405 | 0.656260498 | 4 | S2_S1 | 0.978405 | 0.656260498 | −0.322144503 | 0.817332749 |
| M-Q92616 | 60 | 436.67 | 0 | 0.88364 | 0.77414 | 0 | 1 | S1_M | −0.1095 | −0.88364 | −0.77414 | NA |
| M-Q969V3 | 56.67 | 80 | 13.33 | 0.74208 | 0.88813 | 0.392126222 | 1 | S1_M | 0.14605 | −0.349953779 | −0.496003779 | NA |
| M-Q96AA3 | 0 | 20 | 26.67 | 0 | 0.879485 | 0.765632579 | 4 | S2_S1 | 0.879485 | 0.765632579 | −0.113852421 | 0.82255879 |
| M-Q96CW5 | 16.67 | 90 | 76.67 | 0.442045 | 0.996675 | 0.876803501 | 2 | S2_S1_M | 0.55463 | 0.434758501 | −0.119871490 | 0.494694251 |
| M-Q96D53 | 0 | 50 | 33.33 | 0 | 0.971175 | 0.89537016 | 4 | S2_S1 | 0.971175 | 0.89537016 | −0.07580484 | 0.93327258 |
| M-Q96EC8 | 40 | 20 | 13.33 | 0.970245 | 0.810065 | 0.658644009 | 2 | S2_S1_M | −0.16018 | −0.311600991 | −0.151420991 | −0.235890496 |
| M-Q96ER3 | 0 | 43.33 | 33.33 | 0 | 0.678155 | 0.884736465 | 4 | S2_S1 | 0.678155 | 0.884736465 | 0.206581465 | 0.781445732 |
| M-Q96HR9 | 0 | 0 | 23.33 | 0 | 0 | 0.802828582 | 6 | S2 | NA | 0.802828582 | 0.802828582 | NA |
| M-Q96HW7 | 0 | 20 | 26.67 | 0 | 0.57119 | 0.796652353 | 4 | S2_S1 | 0.57119 | 0.796652353 | 0.225462353 | 0.683921177 |
| M-Q99805 | 0 | 63.33 | 16.67 | 0 | 0.73237 | 0.370049601 | 3 | S1 | 0.73237 | NA | −0.362320399 | NA |
| M-Q9BQ95 | 0 | 23.33 | 0 | 0 | 0.979675 | 0 | 3 | S1 | 0.979675 | NA | −0.979675 | NA |
| M-Q9BQT8 | 6.67 | 20 | 20 | 0.216335 | 0.67231 | 0.765389969 | 4 | S2_S1 | 0.455975 | 0.549054969 | 0.093079969 | 0.502514984 |
| M-Q9BSJ2 | 30 | 163.33 | 130 | 0.932105 | 0.97279 | 0.919790275 | 2 | S2_S1_M | 0.040685 | −0.012314725 | −0.052999725 | 0.014185138 |
| M-Q9BTY2 | 0 | 40 | 13.33 | 0 | 0.945855 | 0.380259188 | 3 | S1 | 0.945855 | NA | −0.565595812 | NA |
| M-Q9BV40 | 90 | 0 | 0 | 0.99369 | 0 | 0 | 5 | M | −0.99369 | −0.99369 | NA | −0.99369 |
| M-Q9BW92 | 3.33 | 40 | 26.67 | 0.0309745 | 0.687315 | 0.864055253 | 4 | S2_S1 | 0.6563405 | 0.833080753 | 0.176740253 | 0.744710626 |
| M-Q9BYC5 | 50 | 0 | 0 | 0.9715 | 0 | 0 | 5 | M | −0.9715 | −0.9715 | NA | −0.9715 |
| M-Q9C0D9 | 0 | 23.33 | 6.67 | 0 | 0.979675 | 0.439888269 | 3 | S1 | 0.979675 | NA | −0.539786731 | NA |
| M-Q9C0E2 | 0 | 36.67 | 6.67 | 0 | 0.956505 | 0.439888018 | 3 | S1 | 0.956505 | NA | −0.516616982 | NA |
| M-Q9GZM5 | 6.67 | 26.67 | 20 | 0.267095 | 0.952425 | 0.566670684 | 4 | S2_S1 | 0.68533 | 0.299575684 | −0.385754316 | 0.492452842 |
| M-Q9H0V9 | 40 | 0 | 0 | 0.97806 | 0 | 0 | 5 | M | −0.97806 | −0.97806 | NA | −0.97806 |
| M-Q9H2J7 | 0 | 30 | 6.67 | 0 | 0.99197 | 0.123398452 | 3 | S1 | 0.99197 | NA | −0.868571549 | NA |
| M-Q9H583 | 32 | 230 | 0 | 0.84819 | 0.878565 | 0 | 1 | S1_M | 0.030375 | −0.84819 | −0.878565 | NA |
| M-Q9H7F0 | 0 | 70 | 23.33 | 0 | 0.995995 | 0.728805922 | 4 | S2_S1 | 0.995995 | 0.728805922 | −0.267189078 | 0.862400461 |
| M-Q9H845 | 0 | 60 | 0 | 0 | 0.92258 | 0 | 3 | S1_M | 0.92258 | NA | −0.92258 | NA |
| M-Q9H8M5 | 0 | 30 | 0 | 0 | 0.99197 | 0 | 3 | S1 | 0.99197 | NA | −0.99197 | NA |
| M-Q9NQC3 | 0 | 60 | 106.67 | 0 | 0.722405 | 0.936913049 | 4 | S2_S1 | 0.722405 | 0.936913049 | 0.214508040 | 0.829659024 |
| M-Q9NVH2 | 0 | 26.67 | 16.67 | 0 | 0.93217 | 0.724122415 | 4 | S2_S1 | 0.93217 | 0.724122415 | −0.208047586 | 0.828146207 |
| M-Q9NVI1 | 136.67 | 373.33 | 270 | 0.906635 | 0.862235 | 0.778646942 | 2 | S2_S1_M | −0.0444 | −0.127988058 | −0.083588058 | −0.086194029 |
| M-Q9NX47 | 40 | 0 | 0 | 0.986215 | 0 | 0 | 5 | M | −0.986215 | −0.986215 | NA | −0.986215 |
| M-Q9P2R7 | 23.33 | 50 | 30 | 0.80607 | 0.88322 | 0.699898649 | 2 | S2_S1_M | 0.07715 | −0.106171351 | −0.183321351 | −0.014510676 |
| M-Q9UBF2 | 0 | 70 | 40 | 0 | 0.959285 | 0.553667697 | 4 | S2_S1 | 0.959285 | 0.553667697 | −0.405617303 | 0.756476349 |
| M-Q9UBU6 | 0 | 13.33 | 23.33 | 0 | 0.755025 | 0.88724416 | 4 | S2_S1 | 0.755025 | 0.88724416 | 0.13221916 | 0.82113458 |
| M-Q9UDR5 | 0 | 23.33 | 30 | 0 | 0.80246 | 0.872554752 | 4 | S2_S1 | 0.80246 | 0.872554752 | 0.070094752 | 0.837507376 |
| M-Q9UI26 | 30 | 93.33 | 40 | 0.991835 | 0.841075 | 0.824692731 | 2 | S2_S1_M | −0.15076 | −0.167142269 | −0.016382269 | −0.158951135 |
| M-Q9UKV5 | 6.67 | 63.33 | 33.33 | 0.13596 | 0.99354 | 0.521758093 | 4 | S2_S1 | 0.85758 | 0.385798093 | −0.471781907 | 0.621689047 |
| M-Q9ULF5 | 0 | 56.67 | 0 | 0 | 0.868735 | 0 | 3 | S1 | 0.868735 | NA | −0.868735 | NA |
| M-Q9ULX6 | 0 | 26.67 | 46.67 | 0 | 0.66 | 0.875990693 | 4 | S2_S1 | 0.66 | 0.875990693 | 0.215990693 | 0.767995346 |
| M-Q9Y312 | 13.33 | 30 | 43.33 | 0.435405 | 0.571505 | 0.895743362 | 2 | S2_S1_M | 0.1361 | 0.460338362 | 0.324238362 | 0.298219181 |
| M-Q9Y4R8 | 46.67 | 196.67 | 70 | 0.874625 | 0.959725 | 0.771203374 | 2 | S2_S1_M | 0.0851 | −0.103421626 | −0.188521626 | −0.009160813 |
| M-Q9Y5Y0 | 0 | 36.67 | 23.33 | 0 | 0.979255 | 0.645491061 | 1 | S2_S1 | 0.979255 | 0.645491061 | −0.33376394 | 0.81237303 |
| M-Q9Y6E2 | 0 | 0 | 23.33 | 0 | 0 | 0.863182181 | 6 | S2 | NA | 0.863182181 | 0.863182181 | NA |
| N-O43818 | 83.33 | 116.67 | 130 | 0.773845 | 0.950105 | 0.930584399 | 2 | S2_S1_M | 0.17626 | 0.156739399 | −0.019520601 | 0.1664997 |
| N-O75683 | 40 | 56.67 | 33.33 | 0.717255 | 0.854285 | 0.799216309 | 2 | S2_S1_M | 0.13703 | 0.081961309 | −0.055068691 | 0.109495654 |
| N-P11940 | 60 | 53.33 | 73.33 | 0.744345 | 0.822355 | 0.868317965 | 2 | S2_S1_M | 0.07801 | 0.123972965 | 0.045962965 | 0.100991482 |
| N-P16989 | 16.67 | 66.67 | 53.33 | 0.512765 | 0.870065 | 0.827197104 | 2 | S2_S1_M | 0.3573 | 0.314432104 | −0.042867897 | 0.335866052 |
| N-P19784 | 38.67 | 133.33 | 70 | 0.88151 | 0.891885 | 0.937524134 | 2 | S2_S1_M | 0.010375 | 0.056014134 | 0.045639134 | 0.033194567 |
| N-P67870 | 12 | 43.33 | 23.33 | 0.56884 | 0.85307 | 0.886803948 | 2 | S2_S1_M | 0.28423 | 0.317963948 | 0.033733948 | 0.301096974 |
| N-P68400 | 36.67 | 30 | 13.33 | 0.935835 | 0.816805 | 0.650644221 | 2 | S2_S1_M | −0.11903 | −0.28519078 | −0.16616078 | −0.20211039 |
| N-Q13283 | 0 | 633.33 | 150.33 | 0 | 0.961845 | 0.97665813 | 4 | S2_S1 | 0.961845 | 0.97665813 | 0.01481313 | 0.969251565 |
| N-Q13310 | 96.67 | 113.33 | 100 | 0.76034 | 0.93303 | 0.923100023 | 2 | S2_S1_M | 0.17269 | 0.162760023 | −0.009929977 | 0.167725012 |
| N-Q15435 | 53.33 | 0 | 0 | 0.991925 | 0 | 0 | 5 | M | −0.991925 | −0.991925 | NA | −0.991925 |
| N-Q6PKG0 | 103.33 | 82 | 86.67 | 0.756 | 0.871 | 0.86893733 | 2 | S2_S1_M | 0.115 | 0.11293733 | −0.00206267 | 0.113968665 |
| N-Q86U42 | 10 | 18 | 7.33 | 0.381655 | 0.83023 | 0.427408997 | 1 | S1_M | 0.448575 | 0.045753997 | −0.402821004 | NA |
| N-Q8NCA5 | 20 | 46.67 | 36.67 | 0.586115 | 0.9648 | 0.96053836 | 2 | S2_S1_M | 0.378685 | 0.37442336 | −0.004261641 | 0.37655418 |
| N-Q8TAD8 | 14.67 | 19.33 | 66.67 | 0.766565 | 0.85822 | 0.909115123 | 2 | S2_S1_M | 0.091655 | 0.142550123 | 0.050895123 | 0.117102561 |
| N-Q92900 | 3.33 | 26.67 | 56.67 | 0.055835 | 0.74484 | 0.876533636 | 4 | S2_S1 | 0.689005 | 0.820698636 | 0.131693636 | 0.754851818 |
| N-Q9BQ75 | 20 | 40 | 6.67 | 0.708235 | 0.91884 | 0.207981733 | 1 | S1_M | 0.210605 | −0.500253268 | −0.710858268 | NA |
| N-Q9HCE1 | 56.67 | 23.33 | 33.33 | 0.83052 | 0.790575 | 0.863336472 | 2 | S2_S1_M | −0.039945 | 0.032816472 | 0.072761472 | 0.003564264 |
| N-Q9UN86 | 0 | 150.67 | 194.33 | 0 | 0.938345 | 0.979066836 | 4 | S2_S1 | 0.938345 | 0.979066836 | 0.040721836 | 0.958705918 |
| nsp1-O60220 | 143.33 | 0 | 0 | 0.852785 | 0 | 0 | 5 | M | −0.852785 | −0.852785 | NA | −0.852785 |
| nsp1-P09884 | 0 | 233.33 | 33.33 | 0 | 0.842755 | 0.985632296 | 4 | S2_S1 | 0.842755 | 0.985632296 | 0.142877296 | 0.914193648 |
| nsp1-P40763 | 50 | 0 | 0 | 0.9743 | 0 | 0 | 5 | M | −0.9743 | −0.9743 | NA | −0.9743 |
| nsp1-P42345 | 33.33 | 0 | 0 | 0.80987 | 0 | 0 | 5 | M | −0.80987 | −0.80987 | NA | −0.80987 |
| nsp1-P49642 | 0 | 70 | 33.33 | 0 | 0.82227 | 0.985634344 | 4 | S2_S1 | 0.82227 | 0.985634344 | 0.163364344 | 0.903952172 |
| nsp1-P49643 | 0 | 160 | 46.67 | 0 | 0.8245 | 0.996987596 | 4 | S2_S1 | 0.8245 | 0.996987596 | 0.172487596 | 0.910743798 |
| nsp1-Q05516 | 153.33 | 0 | 0 | 0.992445 | 0 | 0 | 5 | M | −0.992445 | −0.992445 | NA | −0.992445 |
| nsp1-Q14181 | 0 | 93.33 | 40 | 0 | 0.996645 | 0.806839244 | 4 | S2_S1 | 0.996645 | 0.806839244 | −0.189805756 | 0.901742122 |
| nsp1-Q8NBJ5 | 0 | 0 | 73.33 | 0 | 0 | 0.897061987 | 6 | S2 | NA | 0.897061987 | 0.897061987 | NA |
| nsp1-Q99959 | 0 | 0 | 430 | 0 | 0 | 0.982292676 | 6 | S2 | NA | 0.982292676 | 0.982292676 | NA |
| nsp10-O94973 | 0 | 23.33 | 56.67 | 0 | 0.717935 | 0.995564065 | 4 | S2_S1 | 0.717935 | 0.995564065 | 0.277629065 | 0.856749533 |
| nsp10-P28330 | 120 | 0 | 0 | 0.94001 | 0 | 0 | 5 | M | −0.94001 | −0.94001 | NA | −0.94001 |
| nsp10-P55789 | 0 | 3.56 | 46.67 | 0 | 0.437515 | 0.982686408 | 6 | S2 | NA | 0.982686408 | 0.545171408 | NA |
| nsp10-Q6Q0C0 | 0 | 123.33 | 10 | 0 | 0.992795 | 0.496522731 | 4 | S2_S1 | 0.992795 | 0.496522731 | −0.49627227 | 0.744658865 |
| nsp10-Q969X5 | 0 | 193.33 | 146.67 | 0 | 0.932575 | 0.956119758 | 4 | S2_S1 | 0.932575 | 0.956119758 | 0.023544758 | 0.944347379 |
| nsp10-Q96CW1 | 0 | 16.67 | 30 | 0 | 0.53798 | 0.981452942 | 4 | S2_S1 | 0.53798 | 0.981452942 | 0.443472942 | 0.759716471 |
| nsp10-Q9BZH6 | 46.67 | 0 | 0 | 0.987275 | 0 | 0 | 5 | M | −0.987275 | −0.987275 | NA | −0.987275 |
| nsp10-Q9C026 | 30 | 0 | 0 | 0.776755 | 0 | 0 | 5 | M | −0.776755 | −0.776755 | NA | −0.776755 |
| nsp10-Q9HAV7 | 0 | 30 | 26.67 | 0 | 0.760685 | 0.983293541 | 4 | S2_S1 | 0.760685 | 0.983293541 | 0.222608541 | 0.87198927 |
| nsp11-O14734 | 30 | 30 | 20 | 0.83477 | 0.3202 | 0.349895739 | 5 | M | −0.51457 | −0.484874262 | NA | −0.499722131 |
| nsp11-O75347 | 5.45 | 30 | 14.67 | 0.628805 | 0.572815 | 0.849172351 | 2 | S2_S1_M | −0.05599 | 0.220367351 | 0.276357351 | 0.082188675 |
| nsp11-Q92624 | 16.67 | 73.33 | 16.67 | 0.633205 | 0.92753 | 0.63550932 | 2 | S2_S1_M | 0.294325 | 0.002304319 | −0.292020681 | 0.14831466 |
| nsp11-Q9C0D3 | 0 | 46.67 | 76.67 | 0 | 0.94772 | 0.723916985 | 4 | S2_S1 | 0.94772 | 0.723916985 | −0.223803016 | 0.835818492 |
| nsp13-A7MCY6 | 3.33 | 10 | 63.33 | 0.342755 | 0.592685 | 0.992644762 | 4 | S2_S1 | 0.24993 | 0.649889762 | 0.399959762 | 0.449909881 |
| nsp13-O14578 | 0 | 0 | 60 | 0 | 0 | 0.943657438 | 6 | S2 | NA | 0.943657438 | 0.943657438 | NA |
| nsp13-O14639 | 0 | 53.33 | 0 | 0 | 0.87394 | 0 | 3 | S1 | 0.87394 | NA | −0.87394 | NA |
| nsp13-O14908 | 3.33 | 66.67 | 0 | 0.11038 | 0.925455 | 0 | 3 | S1 | 0.815075 | NA | −0.925455 | NA |
| nsp13-O60237 | 6.67 | 40 | 0 | 0.265685 | 0.709335 | 0 | 3 | S1 | 0.44365 | NA | −0.709335 | NA |
| nsp13-O60784 | 20 | 153.33 | 16.67 | 0.51791 | 0.90991 | 0.263020733 | 1 | S1_M | 0.392 | −0.254889268 | −0.646889268 | NA |
| nsp13-O75381 | 6.67 | 30 | 0 | 0.497755 | 0.76976 | 0 | 1 | S1_M | 0.272005 | −0.497755 | −0.76976 | NA |
| nsp13-O75506 | 0 | 30 | 43.33 | 0 | 0.75879 | 0.925751307 | 4 | S2_S1 | 0.75879 | 0.925751307 | 0.166961307 | 0.842270654 |
| nsp13-O95613 | 923.33 | 1563.33 | 1810 | 0.976445 | 0.97516 | 0.985927969 | 2 | S2_S1_M | −0.001285 | 0.009482969 | 0.010767969 | 0.004098985 |
| nsp13-O95684 | 3.33 | 30 | 20 | 0.342755 | 0.76578 | 0.81578518 | 4 | S2_S1 | 0.423025 | 0.47303018 | 0.050005179 | 0.44802759 |
| nsp13-P06396 | 16.67 | 46.67 | 0 | 0.31461 | 0.874975 | 0 | 3 | S1 | 0.560365 | NA | −0.874975 | NA |
| nsp13-P09493 | 103.33 | 170 | 20 | 0.88494 | 0.905475 | 0.263786409 | 1 | S1_M | 0.020535 | −0.621153591 | −0.641688591 | NA |
| nsp13-P13861 | 156.67 | 103.33 | 200 | 0.938245 | 0.89999 | 0.948928606 | 2 | S2_S1_M | −0.038255 | 0.010683606 | 0.048938606 | −0.013785697 |
| nsp13-P14649 | 40 | 93.33 | 13.33 | 0.87596 | 0.928375 | 0.316990661 | 1 | S1_M | 0.052415 | −0.558969339 | −0.611384339 | NA |
| nsp13-P17612 | 33.33 | 60 | 53.33 | 0.912545 | 0.93384 | 0.940160587 | 2 | S2_S1_M | 0.021295 | 0.027615587 | 0.006320587 | 0.024455294 |
| nsp13-P28289 | 26.67 | 103.33 | 13.33 | 0.537 | 0.85972 | 0.234827413 | 1 | S1_M | 0.32272 | −0.302172588 | −0.624892588 | NA |
| nsp13-P31323 | 30 | 20 | 66.67 | 0.97749 | 0.770075 | 0.991595753 | 2 | S2_S1_M | −0.207415 | 0.014105753 | 0.221520753 | −0.096654624 |
| nsp13-P35241 | 0 | 40 | 70 | 0 | 0.91847 | 0.956014158 | 4 | S2_S1 | 0.91847 | 0.956014158 | 0.037544158 | 0.937242079 |
| nsp13-P49454 | 53.33 | 6.67 | 200 | 0.94142 | 0.440075 | 0.936920322 | 7 | S2_M | −0.501345 | −0.004499678 | 0.496845322 | NA |
| nsp13-P67936 | 150 | 223.33 | 40 | 0.934255 | 0.943055 | 0.355544634 | 1 | S1_M | 0.0088 | −0.578710366 | −0.587510366 | NA |
| nsp13-Q04724 | 0 | 33.33 | 43.33 | 0 | 0.96769 | 0.984586415 | 4 | S2_S1 | 0.96769 | 0.984586415 | 0.016896415 | 0.976138208 |
| nsp13-Q04726 | 0 | 86.67 | 180 | 0 | 0.926085 | 0.966813497 | 4 | S2_S1 | 0.926085 | 0.966813497 | 0.040728497 | 0.946449248 |
| nsp13-Q08117 | 0 | 20 | 23.33 | 0 | 0.799665 | 0.811215516 | 4 | S2_S1 | 0.799665 | 0.811215516 | 0.011550516 | 0.805440258 |
| nsp13-Q08378 | 285.33 | 193 | 850 | 0.954305 | 0.943315 | 0.964369412 | 2 | S2_S1_M | −0.01099 | 0.010064412 | 0.021054412 | −0.000462794 |
| nsp13-Q08379 | 353.33 | 483.33 | 773.33 | 0.955925 | 0.950515 | 0.976155544 | 2 | S2_S1_M | −0.00541 | 0.020230544 | 0.025640544 | 0.007410272 |
| nsp13-Q12965 | 96.67 | 446.67 | 30 | 0.93924 | 0.99351 | 0.507755661 | 2 | S2_S1_M | 0.05427 | −0.431484339 | −0.485754339 | −0.18860717 |
| nsp13-Q13045 | 46.67 | 206.67 | 6.67 | 0.53926 | 0.87053 | 0.180792005 | 1 | S1_M | 0.33127 | −0.358467996 | −0.689737996 | NA |
| nsp13-Q14789 | 10 | 360 | 900 | 0.58494 | 0.94004 | 0.992802271 | 2 | S2_S1_M | 0.3551 | 0.407862271 | 0.052762271 | 0.381481135 |
| nsp13-Q15154 | 290 | 470 | 260 | 0.85182 | 0.876465 | 0.848144227 | 2 | S2_S1_M | 0.024645 | −0.003675773 | −0.028320773 | 0.010484614 |
| nsp13-Q16881 | 140 | 0 | 0 | 0.983335 | 0 | 0 | 5 | M | −0.983335 | −0.983335 | NA | −0.983335 |
| nsp13-Q4V328 | 6.67 | 136.67 | 310 | 0.439925 | 0.84276 | 0.994907985 | 2 | S2_S1_M | 0.402835 | 0.554982985 | 0.152147985 | 0.478908992 |
| nsp13-Q5VT06 | 10 | 46.67 | 56.67 | 0.31597 | 0.70424 | 0.933779965 | 4 | S2_S1 | 0.38827 | 0.617809965 | 0.229539965 | 0.503039983 |
| nsp13-Q5VU43 | 206.67 | 120 | 236.67 | 0.99429 | 0.93966 | 0.989562196 | 2 | S2_S1_M | −0.05463 | −0.004727804 | 0.049902196 | −0.029678902 |
| nsp13-Q5VUJ6 | 0 | 33.33 | 0 | 0 | 0.8676 | 0 | 3 | S1 | 0.8676 | NA | −0.8676 | NA |
| nsp13-Q66GS9 | 26.67 | 40 | 63.33 | 0.7639 | 0.969495 | 0.987646067 | 2 | S2_S1_M | 0.205595 | 0.223746067 | 0.018151067 | 0.214670534 |
| nsp13-Q6ZVM7 | 6.67 | 110 | 6.67 | 0.23647 | 0.963405 | 0.30165288 | 3 | S1 | 0.726935 | NA | −0.66175212 | NA |
| nsp13-Q76N32 | 16.67 | 0 | 30 | 0.581 | 0 | 0.774852108 | 7 | S2_M | −0.581 | 0.193852108 | 0.774852108 | NA |
| nsp13-Q7Z406 | 266.67 | 880 | 63.33 | 0.77439 | 0.85493 | 0.204616775 | 1 | S1_M | 0.08054 | −0.569773226 | −0.650313226 | NA |
| nsp13-Q7Z7A1 | 0 | 0 | 50 | 0 | 0 | 0.994958704 | 6 | S2 | NA | 0.994958704 | 0.994958704 | NA |
| nsp13-Q8IUD2 | 333.33 | 36.67 | 240 | 0.993565 | 0.78437 | 0.995359064 | 2 | S2_S1_M | −0.209195 | 0.001794064 | 0.210989064 | −0.103700468 |
| nsp13-Q8IWJ2 | 80 | 0 | 46.67 | 0.94573 | 0 | 0.99369356 | 7 | S2_M | −0.94573 | 0.04796356 | 0.99369356 | NA |
| nsp13-Q8N3C7 | 0 | 30 | 36.67 | 0 | 0.776945 | 0.978472336 | 4 | S2_S1 | 0.776945 | 0.978472336 | 0.201527336 | 0.877708668 |
| nsp13-Q8N4C6 | 43.33 | 360 | 690 | 0.993405 | 0.842755 | 0.995791597 | 2 | S2_S1_M | −0.15065 | 0.002386597 | 0.153036597 | −0.074131701 |
| nsp13-Q8N8E3 | 13.33 | 3.33 | 23.33 | 0.589445 | 0.342755 | 0.807159418 | 7 | S2_M | −0.24669 | 0.217714418 | 0.464404418 | NA |
| nsp13-Q8NDN9 | 36.67 | 0 | 0 | 0.88797 | 0 | 0 | 5 | M | −0.88797 | −0.88797 | NA | −0.88797 |
| nsp13-Q8TD10 | 83.33 | 86.67 | 180 | 0.94006 | 0.934175 | 0.99088498 | 2 | S2_S1_M | −0.005885 | 0.05082498 | 0.05670998 | 0.02246999 |
| nsp13-Q8WXW3 | 6.67 | 43.33 | 6.67 | 0.296525 | 0.750145 | 0.305252195 | 3 | S1 | 0.45362 | NA | −0.444892806 | NA |
| nsp13-Q92614 | 120 | 576.67 | 26.67 | 0.764855 | 0.93837 | 0.241423284 | 1 | S1_M | 0.173515 | −0.523431717 | −0.696946717 | NA |
| nsp13-Q92995 | 10 | 30 | 103.33 | 0.5891 | 0.97269 | 0.993757226 | 2 | S2_S1_M | 0.38359 | 0.404657226 | 0.021067226 | 0.394123613 |
| nsp13-Q96CN9 | 0 | 4 | 96.67 | 0 | 0.327095 | 0.936680786 | 6 | S2 | NA | 0.936680786 | 0.609585786 | NA |
| nsp13-Q96II8 | 26.67 | 230 | 0 | 0.33355 | 0.95438 | 0 | 3 | S1 | 0.62083 | NA | −0.95438 | NA |
| nsp13-Q96N16 | 0 | 103.33 | 146.67 | 0 | 0.98623 | 0.993983496 | 4 | S2_S1 | 0.98623 | 0.993983496 | 0.007753495 | 0.990106748 |
| nsp13-Q96SN8 | 326.67 | 176 | 626.67 | 0.96175 | 0.954075 | 0.969653624 | 2 | S2_S1_M | −0.007675 | 0.007903623 | 0.015578623 | 0.000114312 |
| nsp13-Q99996 | 548 | 573.33 | 1090 | 0.99493 | 0.93854 | 0.995406905 | 2 | S2_S1_M | −0.05639 | 0.000476905 | 0.056866905 | −0.027956548 |
| nsp13-Q9BQQ3 | 13.33 | 36.67 | 53.33 | 0.64546 | 0.979555 | 0.993435156 | 2 | S2_S1_M | 0.334095 | 0.347975156 | 0.013880156 | 0.341035078 |
| nsp13-Q9BQS8 | 213.33 | 0 | 20 | 0.98596 | 0 | 0.691586651 | 7 | S2_M | −0.98596 | −0.29437335 | 0.691586651 | NA |
| nsp13-Q9BV19 | 0 | 20 | 40 | 0 | 0.968045 | 0.966028423 | 4 | S2_S1 | 0.968045 | 0.966028423 | −0.002016578 | 0.967036711 |
| nsp13-Q9BV73 | 256.67 | 1060 | 1510 | 0.939265 | 0.988335 | 0.995358917 | 2 | S2_S1_M | 0.04907 | 0.056093917 | 0.007023917 | 0.052581958 |
| nsp13-Q9BZF9 | 60 | 293.33 | 20 | 0.6013 | 0.90756 | 0.380534105 | 1 | S1_M | 0.30626 | −0.220765896 | −0.527025896 | NA |
| nsp13-Q9C0B0 | 33.33 | 0 | 0 | 0.97038 | 0 | 0 | 5 | M | −0.97038 | −0.97038 | NA | −0.97038 |
| nsp13-Q9H0E2 | 26.67 | 60 | 3.33 | 0.66643 | 0.92599 | 0.074477515 | 1 | S1_M | 0.25956 | −0.591952486 | −0.851512486 | NA |
| nsp13-Q9UHD2 | 3.33 | 10 | 70 | 0.342755 | 0.592685 | 0.996985298 | 4 | S2_S1 | 0.24993 | 0.654230298 | 0.404300298 | 0.452080149 |
| nsp13-Q9UJC3 | 10 | 123.33 | 240 | 0.58494 | 0.842755 | 0.997024041 | 2 | S2_S1_M | 0.257815 | 0.412084041 | 0.154269041 | 0.33494952 |
| nsp13-Q9ULV0 | 0 | 96.67 | 0 | 0 | 0.697205 | 0 | 3 | S1 | 0.697205 | NA | −0.697205 | NA |
| nsp13-Q9UM54 | 533.33 | 414.67 | 136.67 | 0.84517 | 0.889335 | 0.254120161 | 1 | S1_M | 0.044165 | −0.591049839 | −0.635214839 | NA |
| nsp13-Q9UNZ2 | 23.33 | 0 | 0 | 0.96912 | 0 | 0 | 5 | M | −0.96912 | −0.96912 | NA | −0.96912 |
| nsp13-Q9UPN4 | 66.67 | 240 | 30 | 0.848445 | 0.929395 | 0.786584071 | 2 | S2_S1_M | 0.08095 | −0.06186093 | −0.14281093 | 0.009544535 |
| nsp13-Q9UPQ0 | 0 | 86.67 | 0 | 0 | 0.94774 | 0 | 3 | S1 | 0.94774 | NA | −0.94774 | NA |
| nsp13-Q9Y2I6 | 186.67 | 173.33 | 453.33 | 0.99228 | 0.842755 | 0.993895285 | 2 | S2_S1_M | −0.149525 | 0.001615284 | 0.151140285 | −0.073954858 |
| nsp13-Q9Y4I1 | 86.67 | 603.33 | 20 | 0.790445 | 0.89404 | 0.264800133 | 1 | S1_M | 0.103595 | −0.525644867 | −0.629239867 | NA |
| nsp13-Q9Y608 | 53.33 | 146.67 | 20 | 0.795345 | 0.886585 | 0.256396267 | 1 | S1_M | 0.09124 | −0.538948734 | −0.630188734 | NA |
| nsp14-O95071 | 83.33 | 0 | 0 | 0.713995 | 0 | 0 | 5 | M | −0.713995 | −0.713995 | NA | −0.713995 |
| nsp14-O95714 | 0 | 333.33 | 0 | 0 | 0.98908 | 0 | 3 | S1 | 0.98908 | NA | −0.98908 | NA |
| nsp14-P04637 | 67.2 | 0 | 0 | 0.90646 | 0 | 0 | 5 | M | −0.90646 | −0.90646 | NA | −0.90646 |
| nsp14-P06280 | 0 | 156.67 | 256.67 | 0 | 0.901705 | 0.920568789 | 4 | S2_S1 | 0.901705 | 0.920568789 | 0.018863789 | 0.911136895 |
| nsp14-P12268 | 20 | 63.33 | 183.33 | 0.68699 | 0.84224 | 0.994833804 | 2 | S2_S1_M | 0.15525 | 0.307843804 | 0.152593804 | 0.231546902 |
| nsp14-P30153 | 18.55 | 2.4 | 5.87 | 0.861875 | 0.20035 | 0.576866178 | 7 | S2_M | −0.661525 | −0.285008822 | 0.376516178 | NA |
| nsp14-P49959 | 60 | 0 | 0 | 0.89418 | 0 | 0 | 5 | M | −0.89418 | −0.89418 | NA | −0.89418 |
| nsp14-P63151 | 13.33 | 5.33 | 6.67 | 0.87495 | 0.346635 | 0.182525872 | 5 | M | −0.528315 | −0.692424128 | NA | −0.610369564 |
| nsp14-Q5QP82 | 66.67 | 0 | 0 | 0.9942 | 0 | 0 | 5 | M | −0.9942 | −0.9942 | NA | −0.9942 |
| nsp14-Q5T9A4 | 400 | 0 | 0 | 0.866745 | 0 | 0 | 5 | M | −0.866745 | −0.866745 | NA | −0.866745 |
| nsp14-Q92878 | 88 | 0 | 0 | 0.950265 | 0 | 0 | 5 | M | −0.950265 | −0.950265 | NA | −0.950265 |
| nsp14-Q96EN8 | 133.33 | 0 | 0 | 0.995935 | 0 | 0 | 5 | M | −0.995935 | −0.995935 | NA | −0.995935 |
| nsp14-Q96JN8 | 0 | 173.33 | 0 | 0 | 0.93852 | 0 | 3 | S1 | 0.93852 | NA | −0.93852 | NA |
| nsp14-Q9NQX3 | 60 | 0 | 0 | 0.92189 | 0 | 0 | 5 | M | −0.92189 | −0.92189 | NA | −0.92189 |
| nsp14-Q9NXA8 | 0 | 120 | 116.67 | 0 | 0.99539 | 0.996816405 | 4 | S2_S1 | 0.99539 | 0.996816405 | 0.001426405 | 0.996103203 |
| nsp15- | 36.67 | 0 | 0 | 0.96815 | 0 | 0 | 5 | M | −0.96815 | −0.96815 | NA | −0.96815 |
| A0A0B4J1Y9 | | | | | | | | | | | | |
| nsp15-P61970 | 0 | 0 | 23.33 | 0 | 0 | 0.978943 | 6 | S2 | NA | 0.978943 | 0.978943 | NA |
| nsp15-P62330 | 0 | 36.67 | 70 | 0 | 0.8565 | 0.994065746 | 4 | S2_S1 | 0.8565 | 0.994065746 | 0.137565746 | 0.925282873 |
| nsp15-Q9H4P4 | 0 | 0 | 213.33 | 0 | 0 | 0.996780409 | 6 | S2 | NA | 0.996780409 | 0.996780409 | NA |
| nsp16-A3KMH1 | 33.33 | 0 | 0 | 0.84918 | 0 | 0 | 5 | M | −0.84918 | −0.84918 | NA | −0.84918 |
| nsp16-O14972 | 0 | 0 | 23.33 | 0 | 0 | 0.979836157 | 6 | S2 | NA | 0.979836157 | 0.979836157 | NA |
| nsp16-O43933 | 0 | 0 | 73.33 | 0 | 0 | 0.996519388 | 6 | S2 | NA | 0.996519388 | 0.996519388 | NA |
| nsp16-O60232 | 0.9 | 6.29 | 5.91 | 0.12679 | 0.806585 | 0.669762658 | 4 | S2_S1 | 0.679795 | 0.542972658 | −0.136822342 | 0.611383829 |
| nsp16-O60826 | 0 | 33.33 | 196.67 | 0 | 0.770775 | 0.996219731 | 4 | S2_S1 | 0.770775 | 0.996219731 | 0.225444731 | 0.883497365 |
| nsp16-O75382 | 0 | 0 | 66.67 | 0 | 0 | 0.969539135 | 6 | S2 | NA | 0.969539135 | 0.969539135 | NA |
| nsp16-O75564 | 0 | 0 | 30 | 0 | 0 | 0.844073064 | 6 | S2 | NA | 0.844073064 | 0.844073064 | NA |
| nsp16-O75665 | 0 | 0 | 106.67 | 0 | 0 | 0.996852272 | 6 | S2 | NA | 0.996852272 | 0.996852272 | NA |
| nsp16-O95714 | 0 | 0 | 93.33 | 0 | 0 | 0.936058771 | 6 | S2 | NA | 0.936058771 | 0.936058771 | NA |
| nsp16-O95754 | 0 | 0 | 50 | 0 | 0 | 0.995402353 | 6 | S2 | NA | 0.995402353 | 0.995402353 | NA |
| nsp16-O95835 | 20 | 0 | 0 | 0.88447 | 0 | 0 | 5 | M | −0.88447 | −0.88447 | NA | −0.88447 |
| nsp16-P11717 | 33.33 | 0 | 0 | 0.92214 | 0 | 0 | 5 | M | −0.92214 | −0.92214 | NA | −0.92214 |
| nsp16-P28838 | 0 | 430 | 1383.33 | 0 | 0.9944 | 0.96760784 | 4 | S2_S1 | 0.9944 | 0.96760784 | −0.02679216 | 0.98100392 |
| nsp16-P43686 | 43.33 | 0 | 0 | 0.868745 | 0 | 0 | 5 | M | −0.868745 | −0.868745 | NA | −0.868745 |
| nsp16-P51530 | 0 | 26.67 | 206.67 | 0 | 0.561495 | 0.96542669 | 4 | S2_S1 | 0.561495 | 0.96542669 | 0.40393169 | 0.763460845 |
| nsp16-P51659 | 26.4 | 0 | 10.93 | 0.902195 | 0 | 0.310095897 | 5 | M | −0.902195 | −0.592099103 | NA | −0.747147052 |
| nsp16-P54802 | 453.33 | 0 | 0 | 0.994985 | 0 | 0 | 5 | M | −0.994985 | −0.994985 | NA | −0.994985 |
| nsp16-Q05086 | 0 | 0 | 203.33 | 0 | 0 | 0.996602864 | 6 | S2 | NA | 0.996602864 | 0.996602864 | NA |
| nsp16-Q12923 | 0 | 0.8 | 119.1 | 0 | 0.0175725 | 0.91236423 | 6 | S2 | NA | 0.91236423 | 0.89479173 | NA |
| nsp16-Q13043 | 0 | 0 | 110 | 0 | 0 | 0.968447954 | 6 | S2 | NA | 0.968447954 | 0.968447954 | NA |
| nsp16-Q13049 | 0 | 0 | 93.33 | 0 | 0 | 0.994426958 | 6 | S2 | NA | 0.994426958 | 0.994426958 | NA |
| nsp16-Q13188 | 3.33 | 0 | 150 | 0.342755 | 0 | 0.908059395 | 6 | S2 | NA | 0.565304395 | 0.908059395 | NA |
| nsp16-Q13438 | 36.67 | 0 | 3.33 | 0.995965 | 0 | 0.029719584 | 5 | M | −0.995965 | −0.966245416 | NA | −0.981105208 |
| nsp16-Q15345 | 0 | 0 | 23.33 | 0 | 0 | 0.979200709 | 6 | S2 | NA | 0.979200709 | 0.979200709 | NA |
| nsp16-Q15796 | 46 | 0 | 0 | 0.981045 | 0 | 0 | 5 | M | −0.981045 | −0.981045 | NA | −0.981045 |
| nsp16-Q53EZ4 | 0 | 0 | 253.33 | 0 | 0 | 0.856036213 | 6 | S2 | NA | 0.856036213 | 0.856036213 | NA |
| nsp16-Q567U6 | 0 | 23.33 | 170 | 0 | 0.88717 | 0.996513895 | 4 | S2_S1 | 0.88717 | 0.996513895 | 0.109343895 | 0.941841948 |
| nsp16-Q5SVZ6 | 0 | 260 | 766.67 | 0 | 0.99455 | 0.997013028 | 4 | S2_S1 | 0.99455 | 0.997013028 | 0.002463028 | 0.995781514 |
| nsp16-Q5SZL2 | 0 | 6.67 | 406.67 | 0 | 0.30205 | 0.996748048 | 6 | S2 | NA | 0.996748048 | 0.694698048 | NA |
| nsp16-Q5VUJ6 | 0 | 0 | 243.33 | 0 | 0 | 0.981251596 | 6 | S2 | NA | 0.981251596 | 0.98125159 | NA |
| nsp16-Q63ZY3 | 0 | 0 | 113.33 | 0 | 0 | 0.995911983 | 6 | S2 | NA | 0.995911983 | 0.995911983 | NA |
| nsp16-Q6GYQ0 | 0 | 0 | 36.67 | 0 | 0 | 0.978708321 | 6 | S2 | NA | 0.978708321 | 0.978708321 | NA |
| nsp16-Q6IEG0 | 0 | 0 | 33.33 | 0 | 0 | 0.888545334 | 6 | S2 | NA | 0.888545334 | 0.888545334 | NA |
| nsp16-Q6PJI9 | 23.33 | 0 | 0 | 0.931715 | 0 | 0 | 5 | M | −0.931715 | −0.931715 | NA | −0.931715 |
| nsp16-Q6ZU80 | 0 | 0 | 60 | 0 | 0 | 0.946545955 | 6 | S2 | NA | 0.946545955 | 0.946545955 | NA |
| nsp16-Q6ZWJ1 | 0 | 0 | 30 | 0 | 0 | 0.982523358 | 6 | S2 | NA | 0.982523358 | 0.982523358 | NA |
| nsp16-Q70EL1 | 0 | 0 | 116.67 | 0 | 0 | 0.859490098 | 6 | S2 | NA | 0.859490098 | 0.859490098 | NA |
| nsp16-Q7Z3J2 | 0 | 3.33 | 33.33 | 0 | 0.342755 | 0.99060053 | 6 | S2 | NA | 0.99060053 | 0.64784553 | NA |
| nsp16-Q7Z4G1 | 0 | 0 | 20 | 0 | 0 | 0.97198845 | 6 | S2 | NA | 0.97198845 | 0.97198845 | NA |
| nsp16-Q86SQ0 | 0 | 0 | 86.67 | 0 | 0 | 0.915913218 | 6 | S2 | NA | 0.915913218 | 0.915913218 | NA |
| nsp16-Q86W92 | 0 | 0 | 223.33 | 0 | 0 | 0.984180404 | 6 | S2 | NA | 0.984180404 | 0.984180404 | NA |
| nsp16-Q86X10 | 0 | 0 | 50 | 0 | 0 | 0.991607337 | 6 | S2 | NA | 0.991607337 | 0.991607337 | NA |
| nsp16-Q8IUD2 | 0 | 356.67 | 2083.33 | 0 | 0.9633 | 0.960675251 | 4 | S2_S1 | 0.9633 | 0.960675251 | −0.002624749 | 0.961987626 |
| nsp16-Q8IWR1 | 26.67 | 0 | 0 | 0.808845 | 0 | 0 | 5 | M | −0.808845 | −0.808845 | NA | −0.808845 |
| nsp16-Q8N668 | 0 | 0 | 26.67 | 0 | 0 | 0.810656863 | 6 | S2 | NA | 0.810656863 | 0.810656863 | NA |
| nsp16-Q8TEM1 | 0 | 583.33 | 606.67 | 0 | 0.99054 | 0.925377868 | 4 | S2_S1 | 0.99054 | 0.925377868 | −0.065162133 | 0.957958934 |
| nsp16-Q92995 | 653.33 | 0 | 0 | 0.99117 | 0 | 0 | 5 | M | −0.99117 | −0.99117 | NA | −0.99117 |
| nsp16-Q96DZ1 | 133.33 | 0 | 23.33 | 0.893355 | 0 | 0.677399056 | 7 | S2_M | −0.893355 | −0.215955945 | 0.677399056 | NA |
| nsp16-Q96HP0 | 0 | 0 | 76.67 | 0 | 0 | 0.995171398 | 6 | S2 | NA | 0.995171398 | 0.995171398 | NA |
| nsp16-Q96II8 | 0 | 0 | 290 | 0 | 0 | 0.968817445 | 6 | S2 | NA | 0.968817445 | 0.968817445 | NA |
| nsp16-Q96IV0 | 70 | 0 | 0 | 0.980285 | 0 | 0 | 5 | M | −0.980285 | −0.980285 | NA | −0.980285 |
| nsp16-Q96RU2 | 30 | 0 | 0 | 0.97364 | 0 | 0 | 5 | M | −0.97364 | −0.97364 | NA | −0.97364 |
| nsp16-Q9BVQ7 | 0 | 0 | 43.33 | 0 | 0 | 0.990630835 | 6 | S2 | NA | 0.990630835 | 0.990630835 | NA |
| nsp16-Q9GZQ3 | 0 | 0 | 43.33 | 0 | 0 | 0.996497251 | 6 | S2 | NA | 0.996497251 | 0.996497251 | NA |
| nsp16-Q9H000 | 0 | 0 | 96.67 | 0 | 0 | 0.85791191 | 6 | S2 | NA | 0.85791191 | 0.85791191 | NA |
| nsp16-Q9H0H0 | 0 | 10 | 186.67 | 0 | 0.319705 | 0.969170384 | 6 | S2 | NA | 0.969170384 | 0.649465384 | NA |
| nsp16-Q9H4B6 | 0 | 0 | 233.33 | 0 | 0 | 0.934805068 | 6 | S2 | NA | 0.934805068 | 0.934805068 | NA |
| nsp16-Q9NVH2 | 0 | 0 | 176.67 | 0 | 0 | 0.960012505 | 6 | S2 | NA | 0.960012505 | 0.960012505 | NA |
| nsp16-Q9NX08 | 0 | 0 | 10.93 | 0 | 0 | 0.913492843 | 6 | S2 | NA | 0.913492843 | 0.913492843 | NA |
| nsp16-Q9P000 | 0 | 0 | 36.67 | 0 | 0 | 0.986832599 | 6 | S2 | NA | 0.986832599 | 0.986832599 | NA |
| nsp16-Q9P209 | 86.67 | 0 | 3.33 | 0.980135 | 0 | 0.342755123 | 5 | M | −0.980135 | −0.637379877 | NA | −0.808757439 |
| nsp16-Q9P2D0 | 0 | 0 | 180 | 0 | 0 | 0.887081752 | 6 | S2 | NA | 0.887081752 | 0.887081752 | NA |
| nsp16-Q9P2S5 | 0 | 0 | 53.33 | 0 | 0 | 0.993772275 | 6 | S2 | NA | 0.993772275 | 0.993772275 | NA |
| nsp16-Q9UBI1 | 0 | 0 | 40 | 0 | 0 | 0.994676141 | 6 | S2 | NA | 0.994676141 | 0.994676141 | NA |
| nsp16-Q9UHD2 | 0 | 0 | 113.33 | 0 | 0 | 0.865348264 | 6 | S2 | NA | 0.865348264 | 0.865348264 | NA |
| nsp16-Q9UHP3 | 0 | 0 | 100 | 0 | 0 | 0.990190321 | 6 | S2 | NA | 0.990190321 | 0.990190321 | NA |
| nsp16-Q9UKF6 | 0 | 83.33 | 196.67 | 0 | 0.946375 | 0.865984944 | 4 | S2_S1 | 0.946375 | 0.865984944 | −0.080390056 | 0.906179972 |
| nsp16-Q9ULA0 | 110 | 0 | 0 | 0.964395 | 0 | 0 | 5 | M | −0.964395 | −0.964395 | NA | −0.964395 |
| nsp16-Q9UN81 | 0 | 0 | 26.67 | 0 | 0 | 0.920674794 | 6 | S2 | NA | 0.920674794 | 0.920674794 | NA |
| nsp16-Q9Y2D8 | 0 | 10 | 263.33 | 0 | 0.496975 | 0.972204186 | 4 | S2_S1 | 0.496975 | 0.972204186 | 0.475229186 | 0.734589593 |
| nsp16-Q9Y2K2 | 0 | 0 | 63.33 | 0 | 0 | 0.988628258 | 6 | S2 | NA | 0.988628258 | 0.988628258 | NA |
| nsp16-Q9Y2S7 | 6.67 | 66.67 | 10 | 0.113415 | 0.8709 | 0.253465437 | 3 | S1 | 0.757485 | NA | −0.617434563 | NA |
| nsp16-Q9Y305 | 83.33 | 0 | 0 | 0.978815 | 0 | 0 | 5 | M | −0.978815 | −0.978815 | NA | −0.978815 |
| nsp16-Q9Y6G5 | 0 | 0 | 53.33 | 0 | 0 | 0.996204159 | 6 | S2 | NA | 0.996204159 | 0.996204159 | NA |
| nsp2-O00186 | 36.67 | 0 | 0 | 0.99584 | 0 | 0 | 5 | M | −0.99584 | −0.99584 | NA | −0.99584 |
| nsp2-O00303 | 69.33 | 183.33 | 0 | 0.767155 | 0.936365 | 0 | 1 | S1_M | 0.16921 | −0.767155 | −0.936365 | NA |
| nsp2-O00746 | 23.33 | 10 | 0 | 0.878735 | 0.355555 | 0 | 5 | M | −0.52318 | −0.878735 | NA | −0.7009575 |
| nsp2-O14975 | 20 | 30 | 46.67 | 0.55072 | 0.538755 | 0.952901743 | 2 | S2_S1_M | −0.011965 | 0.402181743 | 0.414146743 | 0.195108372 |
| nsp2-O15372 | 43.43 | 28.67 | 3.33 | 0.733135 | 0.857295 | 0.009825276 | 1 | S1_M | 0.12416 | −0.723309725 | −0.847469725 | NA |
| nsp2-O60573 | 155 | 118.4 | 103.33 | 0.75766 | 0.91511 | 0.903416875 | 2 | S2_S1_M | 0.15745 | 0.145756875 | −0.011693126 | 0.151603437 |
| nsp2-O75821 | 23.43 | 33.09 | 0 | 0.672165 | 0.884765 | 0 | 1 | S1_M | 0.2126 | −0.672165 | −0.884765 | NA |
| nsp2-O75822 | 29.33 | 106.67 | 0 | 0.779205 | 0.92797 | 0 | 1 | S1_M | 0.148765 | −0.779205 | −0.92797 | NA |
| nsp2-P00387 | 36.67 | 6.67 | 0 | 0.86857 | 0.13245 | 0 | 5 | M | −0.73612 | −0.86857 | NA | −0.802345 |
| nsp2-P15954 | 26.67 | 0 | 10 | 0.97975 | 0 | 0.221215066 | 5 | M | −0.97975 | −0.758534934 | NA | −0.869142467 |
| nsp2-P16435 | 73.33 | 20 | 33.33 | 0.873805 | 0.55664 | 0.855480885 | 2 | S2_S1_M | −0.317165 | −0.018324116 | 0.298840885 | −0.167744558 |
| nsp2-P52306 | 0 | 46.67 | 120 | 0 | 0.963885 | 0.995817872 | 4 | S2_S1 | 0.963885 | 0.995817872 | 0.031932872 | 0.979851436 |
| nsp2-P60228 | 92 | 44.89 | 0 | 0.774535 | 0.877505 | 0 | 1 | S1_M | 0.10297 | −0.774535 | −0.877505 | NA |
| nsp2-Q10471 | 30 | 0 | 0 | 0.976945 | 0 | 0 | 5 | M | −0.976945 | −0.976945 | NA | −0.976945 |
| nsp2-Q13423 | 36.67 | 0 | 0 | 0.872595 | 0 | 0 | 5 | M | −0.872595 | −0.872595 | NA | −0.872595 |
| nsp2-Q14152 | 51.11 | 71.3 | 0 | 0.761245 | 0.93187 | 0 | 1 | S1_M | 0.170625 | −0.761245 | −0.93187 | NA |
| nsp2-Q15650 | 180 | 0 | 0 | 0.93926 | 0 | 0 | 5 | M | −0.93926 | −0.93926 | NA | −0.93926 |
| nsp2-Q2M389 | 0 | 0 | 36.67 | 0 | 0 | 0.981057591 | 6 | S2 | NA | 0.981057591 | 0.981057591 | NA |
| nsp2-Q5SZL2 | 40 | 0 | 0 | 0.76736 | 0 | 0 | 5 | M | −0.76736 | −0.76736 | NA | −0.76736 |
| nsp2-Q5T1M5 | 0 | 16.67 | 196.67 | 0 | 0.804275 | 0.994028348 | 4 | S2_S1 | 0.804275 | 0.994028348 | 0.189753348 | 0.899151674 |
| nsp2-Q5VT66 | 30 | 0 | 0 | 0.911505 | 0 | 0 | 5 | M | −0.911505 | −0.911505 | NA | −0.911505 |
| nsp2-Q6NUN9 | 70 | 36.67 | 0 | 0.982745 | 0.755435 | 0 | 1 | S1_M | −0.22731 | −0.982745 | −0.755435 | NA |
| nsp2-Q6Y7W6 | 79.08 | 126.82 | 403.33 | 0.884135 | 0.936885 | 0.883612278 | 2 | S2_S1_M | 0.05275 | −0.000522722 | −0.053272722 | 0.026113639 |
| nsp2-Q7L2H7 | 253.33 | 260 | 0 | 0.813735 | 0.98171 | 0 | 1 | S1_M | 0.167975 | −0.813735 | −0.98171 | NA |
| nsp2-Q86UK7 | 36 | 45.44 | 38.5 | 0.741785 | 0.88422 | 0.782745415 | 2 | S2_S1_M | 0.142435 | 0.040960415 | −0.101474585 | 0.091697707 |
| nsp2-Q8N3C0 | 950 | 0 | 0 | 0.915915 | 0 | 0 | 5 | M | −0.915915 | −0.915915 | NA | −0.915915 |
| nsp2-Q8N9N2 | 130 | 0 | 0 | 0.991115 | 0 | 0 | 5 | M | −0.991115 | −0.991115 | NA | −0.991115 |
| nsp2-Q8NBU5 | 63.33 | 0 | 0 | 0.864215 | 0 | 0 | 5 | M | −0.864215 | −0.864215 | NA | −0.864215 |
| nsp2-Q8TF46 | 106.67 | 0 | 0 | 0.99519 | 0 | 0 | 5 | M | −0.99519 | −0.99519 | NA | −0.99519 |
| nsp2-Q8WVC6 | 26.67 | 0 | 0 | 0.872865 | 0 | 0 | 5 | M | −0.872865 | −0.872865 | NA | −0.872865 |
| nsp2-Q96A26 | 50 | 3.33 | 3.33 | 0.889775 | 0.0071725 | 0.005577709 | 5 | M | −0.8826025 | −0.884197292 | NA | −0.883399896 |
| nsp2-Q96B26 | 26.67 | 0 | 0 | 0.726055 | 0 | 0 | 5 | M | −0.726055 | −0.726055 | NA | −0.726055 |
| nsp2-Q96D09 | 193.33 | 0 | 0 | 0.99498 | 0 | 0 | 5 | M | −0.99498 | −0.99498 | NA | −0.99498 |
| nsp2-Q99613 | 46.67 | 40 | 0 | 0.9963 | 0.996585 | 0 | 1 | S1_M | 0.000285 | −0.9963 | −0.996585 | NA |
| nsp2-Q9BQ70 | 190 | 0 | 0 | 0.911145 | 0 | 0 | 5 | M | −0.911145 | −0.911145 | NA | −0.911145 |
| nsp2-Q9C037 | 10 | 120 | 0 | 0.178415 | 0.873945 | 0 | 3 | SI | 0.69553 | NA | −0.873945 | NA |
| nsp2-Q9H1I8 | 216.67 | 0 | 0 | 0.94009 | 0 | 0 | 5 | M | −0.94009 | −0.94009 | NA | −0.94009 |
| nsp2-Q9HD20 | 53.33 | 0 | 0 | 0.95877 | 0 | 0 | 5 | M | −0.95877 | −0.95877 | NA | −0.95877 |
| nsp2-Q9UBQ5 | 33 | 32 | 0 | 0.773085 | 0.86888 | 0 | 1 | S1_M | 0.095795 | −0.773085 | −0.86888 | NA |
| nsp2-Q9UH62 | 23.33 | 0 | 0 | 0.969445 | 0 | 0 | 5 | M | −0.969445 | −0.969445 | NA | −0.969445 |
| nsp2-Q9UPQ9 | 236 | 0 | 0 | 0.868555 | 0 | 0 | 5 | M | −0.868555 | −0.868555 | NA | −0.868555 |
| nsp2-Q9Y262 | 76 | 134 | 0 | 0.733055 | 0.93681 | 0 | 1 | S1_M | 0.203755 | −0.733055 | −0.93681 | NA |
| nsp4-P13674 | 50 | 0 | 16.67 | 0.951615 | 0 | 0.347077058 | 5 | M | −0.951615 | −0.604537943 | NA | −0.778076471 |
| nsp4-P14735 | 0 | 50 | 113.33 | 0 | 0.99431 | 0.959015721 | 4 | S2_S1 | 0.99431 | 0.959015721 | −0.035294279 | 0.976662861 |
| nsp4-P49257 | 116.67 | 6.67 | 0 | 0.884265 | 0.28957 | 0 | 5 | M | −0.594695 | −0.884265 | NA | −0.73948 |
| nsp4-P62072 | 0 | 3.33 | 53.33 | 0 | 0.021763 | 0.980735991 | 6 | S2 | NA | 0.980735991 | 0.958972991 | NA |
| nsp4-P62699 | 0 | 30 | 0 | 0 | 0.991805 | 0 | 3 | S1 | 0.991805 | NA | −0.991805 | NA |
| nsp4-Q13586 | 26.67 | 0 | 0 | 0.969345 | 0 | 0 | 5 | M | −0.969345 | −0.969345 | NA | −0.969345 |
| nsp4-Q2TAA5 | 0 | 40 | 70 | 0 | 0.800615 | 0.863728025 | 4 | S2_S1 | 0.800615 | 0.863728025 | 0.063113025 | 0.832171513 |
| nsp4-Q6VN20 | 0 | 36.67 | 0 | 0 | 0.996385 | 0 | 3 | S1 | 0.996385 | NA | −0.996385 | NA |
| nsp4-Q7L5Y9 | 0 | 26.67 | 0 | 0 | 0.984585 | 0 | 3 | S1 | 0.984585 | NA | −0.984585 | NA |
| nsp4-Q8NBJ7 | 33.33 | 0 | 0 | 0.990575 | 0 | 0 | 5 | M | −0.990575 | −0.990575 | NA | −0.990575 |
| nsp4-Q8NFQ8 | 46.67 | 0 | 0 | 0.89845 | 0 | 0 | 5 | M | −0.89845 | −0.89845 | NA | −0.89845 |
| nsp4-Q8TEM1 | 86.67 | 3.33 | 63.33 | 0.69621 | 0.00199495 | 0.855087349 | 7 | S2_M | −0.69421505 | 0.158877349 | 0.853092399 | NA |
| nsp4-Q92643 | 50 | 6.67 | 30 | 0.914435 | 0.11348 | 0.540710722 | 7 | S2_M | −0.800955 | −0.373724278 | 0.427230722 | NA |
| nsp4-Q969N2 | 40 | 0 | 16.67 | 0.85454 | 0 | 0.341991813 | 5 | M | −0.85454 | −0.512548188 | NA | −0.683544094 |
| nsp4-Q96S59 | 0 | 70 | 0 | 0 | 0.99675 | 0 | 3 | S1 | 0.99675 | NA | −0.99675 | NA |
| nsp4-Q9BSF4 | 0 | 0 | 76.67 | 0 | 0 | 0.993490156 | 6 | S2 | NA | 0.993490156 | 0.993490156 | NA |
| nsp4-Q9H7D7 | 0 | 93.33 | 0 | 0 | 0.964705 | 0 | 3 | S1 | 0.964705 | NA | −0.964705 | NA |
| nsp4-Q9H871 | 0 | 40 | 0 | 0 | 0.9787 | 0 | 3 | S1 | 0.9787 | NA | −0.9787 | NA |
| nsp4-Q9NVH1 | 0 | 0 | 113.33 | 0 | 0 | 0.863433437 | 6 | S2 | NA | 0.863433437 | 0.863433437 | NA |
| nsp4-Q9NWU2 | 0 | 46.67 | 0 | 0 | 0.990345 | 0 | 3 | S1 | 0.990345 | NA | −0.990345 | NA |
| nsp4-Q9Y5J6 | 0 | 0 | 30 | 0 | 0 | 0.982552028 | 6 | S2 | NA | 0.982552028 | 0.982552028 | NA |
| nsp4-Q9Y5J7 | 0 | 0 | 40 | 0 | 0 | 0.956903142 | 6 | S2 | NA | 0.956903142 | 0.956903142 | NA |
| nsp6-O75964 | 3.33 | 40 | 66.67 | 0.010592 | 0.711715 | 0.858632779 | 4 | S2_S1 | 0.701123 | 0.848040779 | 0.146917779 | 0.77458189 |
| nsp6-P25685 | 43.33 | 0 | 0 | 0.911885 | 0 | 0 | 5 | M | −0.911885 | −0.911885 | NA | −0.911885 |
| nsp6-Q15904 | 13.33 | 0 | 50 | 0.51662 | 0 | 0.994553461 | 7 | S2_M | −0.51662 | 0.477933461 | 0.994553461 | NA |
| nsp6-Q99720 | 0 | 63.33 | 50 | 0 | 0.870475 | 0.921106627 | 4 | S2_S1 | 0.870475 | 0.921106627 | 0.050631627 | 0.895790813 |
| nsp6-Q9H7F0 | 0 | 6.67 | 56.67 | 0 | 0.13509 | 0.902762927 | 6 | S2 | NA | 0.902762927 | 0.767672927 | NA |
| nsp6-Q9UDY4 | 23.33 | 0 | 0 | 0.769675 | 0 | 0 | 5 | M | −0.769675 | −0.769675 | NA | −0.769675 |
| nsp7-A8MTT3 | 46.67 | 30 | 16.67 | 0.996545 | 0.983035 | 0.814439289 | 2 | S2_S1_M | −0.01351 | −0.182105712 | −0.168595712 | −0.097807856 |
| nsp7-O00116 | 8 | 90 | 76.67 | 0.58034 | 0.81255 | 0.913245163 | 2 | S2_S1_M | 0.23221 | 0.332905163 | 0.100695163 | 0.282557581 |
| nsp7-O14975 | 36.67 | 10 | 3.33 | 0.89937 | 0.301675 | 0.024969109 | 5 | M | −0.597695 | −0.874400892 | NA | −0.736047946 |
| nsp7-O43169 | 13.33 | 33.33 | 33.33 | 0.46285 | 0.698355 | 0.896755095 | 2 | S2_S1_M | 0.235505 | 0.433905095 | 0.198400095 | 0.334705048 |
| nsp7-O94766 | 40 | 26.67 | 26.67 | 0.77505 | 0.703715 | 0.777879459 | 2 | S2_S1_M | −0.071335 | 0.002829459 | 0.074164459 | −0.034252771 |
| nsp7-O95159 | 23.33 | 10 | 0 | 0.83907 | 0.2099495 | 0 | 5 | M | −0.6291205 | −0.83907 | NA | −0.73409525 |
| nsp7-O95573 | 173.33 | 100 | 43.33 | 0.956415 | 0.80568 | 0.948534466 | 2 | S2_S1_M | −0.150735 | −0.007880534 | 0.142854466 | −0.079307767 |
| nsp7-P00387 | 3.33 | 60 | 73.33 | 0.0394585 | 0.87562 | 0.978174676 | 4 | S2_S1 | 0.8361615 | 0.938716176 | 0.102554676 | 0.887438838 |
| nsp7-P11233 | 23.33 | 33.33 | 23.33 | 0.619915 | 0.67243 | 0.860183243 | 2 | S2_S1_M | 0.052515 | 0.240268243 | 0.187753243 | 0.146391621 |
| nsp7-P21964 | 20 | 73.33 | 20 | 0.759765 | 0.69864 | 0.702615883 | 2 | S2_S1_M | −0.061125 | −0.057149118 | 0.003975882 | −0.059137059 |
| nsp7-P51148 | 0 | 56.67 | 80 | 0 | 0.77073 | 0.939542965 | 4 | S2_S1 | 0.77073 | 0.939542965 | 0.168812965 | 0.855136483 |
| nsp7-P51149 | 0 | 56.67 | 106.67 | 0 | 0.740855 | 0.986362115 | 4 | S2_S1 | 0.740855 | 0.986362115 | 0.245507115 | 0.863608557 |
| nsp7-P61006 | 3.33 | 46.67 | 23.33 | 0.047039 | 0.877235 | 0.772872298 | 4 | S2_S1 | 0.830196 | 0.725833298 | −0.104362702 | 0.778014649 |
| nsp7-P61019 | 0 | 33.33 | 20 | 0 | 0.770655 | 0.81459786 | 4 | S2_S1 | 0.770655 | 0.81459786 | 0.04394286 | 0.79262643 |
| nsp7-P61026 | 3.33 | 23.33 | 30 | 0.056935 | 0.68887 | 0.980721536 | 4 | S2_S1 | 0.631935 | 0.923786536 | 0.291851536 | 0.777860768 |
| nsp7-P61106 | 13.33 | 76.67 | 66.67 | 0.348925 | 0.684125 | 0.875356413 | 4 | S2_S1 | 0.3352 | 0.526431413 | 0.191231413 | 0.430815707 |
| nsp7-P61586 | 0 | 26.67 | 20 | 0 | 0.67556 | 0.7395147 | 4 | S2_S1 | 0.67556 | 0.7395147 | 0.0639547 | 0.70753735 |
| nsp7-P62820 | 0 | 36.67 | 40 | 0 | 0.71914 | 0.962644797 | 4 | S2_S1 | 0.71914 | 0.962644797 | 0.243504797 | 0.840892398 |
| nsp7-P62873 | 3.33 | 16.67 | 26.67 | 0.0137575 | 0.30248 | 0.909766068 | 6 | S2 | NA | 0.896008568 | 0.607286068 | NA |
| nsp7-P63218 | 6.67 | 16.67 | 20 | 0.162845 | 0.47149 | 0.733815783 | 4 | S2_S1 | 0.308645 | 0.570970783 | 0.262325783 | 0.439807892 |
| nsp7-Q12907 | 0 | 60 | 70 | 0 | 0.871285 | 0.862886992 | 4 | S2_S1 | 0.871285 | 0.862886992 | −0.008398008 | 0.867085996 |
| nsp7-Q13724 | 246.67 | 406.67 | 276.67 | 0.90434 | 0.834215 | 0.891165494 | 2 | S2_S1_M | −0.070125 | −0.013174507 | 0.056950493 | −0.041649753 |
| nsp7-Q2TAA5 | 0 | 63.33 | 30 | 0 | 0.9501 | 0.557525176 | 4 | S2_S1 | 0.9501 | 0.557525176 | −0.392574824 | 0.753812588 |
| nsp7-Q53H12 | 253.33 | 273.33 | 210 | 0.852945 | 0.702285 | 0.790614972 | 2 | S2_S1_M | −0.15066 | −0.062330028 | 0.088329972 | −0.106495014 |
| nsp7-Q5JTV8 | 3.33 | 20 | 20 | 0.018931 | 0.743185 | 0.697584025 | 4 | S2_S1 | 0.724254 | 0.678653025 | −0.045600975 | 0.701453513 |
| nsp7-Q5VT66 | 10 | 73.33 | 63.33 | 0.262925 | 0.914985 | 0.969860512 | 4 | S2_S1 | 0.65206 | 0.706935512 | 0.054875512 | 0.679497756 |
| nsp7-Q6P1M0 | 106.67 | 0 | 0 | 0.955085 | 0 | 0 | 5 | M | −0.955085 | −0.955085 | NA | −0.955085 |
| nsp7-Q6P1Q0 | 146.67 | 83.33 | 40 | 0.98912 | 0.895605 | 0.843229772 | 2 | S2_S1_M | −0.093515 | −0.145890229 | −0.052375229 | −0.119702614 |
| nsp7-Q6ZRP7 | 36.67 | 63.33 | 43.33 | 0.968085 | 0.994445 | 0.732162573 | 2 | S2_S1_M | 0.02636 | −0.235922427 | −0.262282427 | −0.104781214 |
| nsp7-Q7LGA3 | 10 | 43.33 | 50 | 0.28665 | 0.904245 | 0.853233417 | 4 | S2_S1 | 0.617595 | 0.566583417 | −0.051011583 | 0.592089209 |
| nsp7-Q8IUR0 | 0 | 20 | 6.67 | 0 | 0.929345 | 0.438749271 | 3 | S1 | 0.929345 | NA | −0.49059573 | NA |
| nsp7-Q8N183 | 0 | 13.33 | 30 | 0 | 0.69781 | 0.980722429 | 4 | S2_S1 | 0.69781 | 0.980722429 | 0.282912429 | 0.839266215 |
| nsp7-Q8N2K0 | 50 | 6.67 | 13.33 | 0.889245 | 0.1209 | 0.356790399 | 5 | M | −0.768345 | −0.532454601 | NA | −0.650399801 |
| nsp7-Q8N9F7 | 40 | 6.67 | 0 | 0.993505 | 0.43991 | 0 | 5 | M | −0.553595 | −0.993505 | NA | −0.77355 |
| nsp7-Q8NBU5 | 86.67 | 70 | 36.67 | 0.86913 | 0.79998 | 0.81621023 | 2 | S2_S1_M | −0.06915 | −0.05291977 | 0.01623023 | −0.061034885 |
| nsp7-Q8NBX0 | 23.33 | 56.67 | 23.33 | 0.813255 | 0.996085 | 0.97433756 | 2 | S2_S1_M | 0.18283 | 0.16108256 | −0.021747441 | 0.17195628 |
| nsp7-Q8WTV0 | 0 | 30 | 26.67 | 0 | 0.98008 | 0.757203124 | 4 | S2_S1 | 0.98008 | 0.757203124 | −0.222876877 | 0.868641562 |
| nsp7-Q8WUY8 | 70 | 40 | 43.33 | 0.970235 | 0.889705 | 0.860142873 | 2 | S2_S1_M | −0.08053 | −0.110092127 | −0.029562127 | −0.095311064 |
| nsp7-Q8WVC6 | 290 | 83.33 | 90 | 0.958145 | 0.8368 | 0.931226168 | 2 | S2_S1_M | −0.121345 | −0.026918833 | 0.094426168 | −0.074131916 |
| nsp7-Q96A26 | 136.67 | 166.67 | 110 | 0.92584 | 0.93852 | 0.874386791 | 2 | S2_S1_M | 0.01268 | −0.051453209 | −0.064133209 | −0.019386605 |
| nsp7-Q96DA6 | 20 | 26.67 | 30 | 0.713645 | 0.7685 | 0.980725063 | 2 | S2_S1_M | 0.054855 | 0.267080063 | 0.212225063 | 0.160967532 |
| nsp7-Q96ER9 | 0 | 26.67 | 3.33 | 0 | 0.9181 | 0.342755242 | 3 | S1 | 0.9181 | NA | −0.575344758 | NA |
| nsp7-Q96KC8 | 0 | 33.33 | 0 | 0 | 0.979895 | 0 | 3 | S1 | 0.979895 | NA | −0.979895 | NA |
| nsp7-Q9BQE4 | 23.33 | 50 | 33.33 | 0.82553 | 0.86263 | 0.850882202 | 2 | S2_S1_M | 0.0371 | 0.025352202 | −0.011747798 | 0.031226101 |
| nsp7-Q9H7Z7 | 196.67 | 60 | 60 | 0.988265 | 0.93241 | 0.877269166 | 2 | S2_S1_M | −0.055855 | −0.110995835 | −0.055140835 | −0.083425417 |
| nsp7-Q9NP72 | 0 | 26.67 | 20 | 0 | 0.54086 | 0.703302544 | 4 | S2_S1 | 0.54086 | 0.703302544 | 0.162442544 | 0.622081272 |
| nsp7-Q9NX40 | 70 | 80 | 76.67 | 0.954545 | 0.79609 | 0.845374481 | 2 | S2_S1_M | −0.158455 | −0.109170519 | 0.049284481 | −0.13381276 |
| nsp7-Q9NYP7 | 0 | 23.33 | 3.33 | 0 | 0.90949 | 0.342755427 | 3 | S1 | 0.90949 | NA | −0.566734573 | NA |
| nsp7-Q9Y3D7 | 6.67 | 33.33 | 13.33 | 0.296865 | 0.8098 | 0.5483636 | 4 | S2_S1 | 0.512935 | 0.2514986 | −0.261436401 | 0.3822168 |
| nsp7-Q9Y5J7 | 26.67 | 10 | 3.33 | 0.716075 | 0.16155 | 0.037183933 | 5 | M | −0.554525 | −0.678891068 | NA | −0.6167080 |
| nsp8-O00566 | 30 | 30 | 26.67 | 0.80071 | 0.886905 | 0.694279586 | 2 | S2_S1_M | 0.086195 | −0.106430414 | −0.192625414 | −0.010117707 |
| nsp8-O15381 | 30 | 30 | 0 | 0.94873 | 0.51182 | 0 | 1 | S1_M | −0.43691 | −0.94873 | −0.51182 | NA |
| nsp8-O60287 | 60 | 133.33 | 90 | 0.875535 | 0.81079 | 0.79329767 | 2 | S2_S1_M | −0.064745 | −0.08223733 | −0.017492331 | −0.073491165 |
| nsp8-O76094 | 253.33 | 336.67 | 336.67 | 0.751585 | 0.860345 | 0.869770328 | 2 | S2_S1_M | 0.10876 | 0.118185328 | 0.009425328 | 0.113472664 |
| nsp8-O95260 | 0 | 140 | 83.33 | 0 | 0.91861 | 0.902146319 | 4 | S2_S1 | 0.91861 | 0.902146319 | −0.016463682 | 0.910378159 |
| nsp8-O95373 | 46.67 | 0 | 0 | 0.86596 | 0 | 0 | 5 | M | −0.86596 | −0.86596 | NA | −0.86596 |
| nsp8-O95707 | 26.67 | 10 | 10 | 0.85579 | 0.590045 | 0.5935402 | 2 | S2_S1_M | −0.265745 | −0.2622498 | 0.0034952 | −0.2639974 |
| nsp8-O96028 | 6.67 | 20 | 20 | 0.24973 | 0.812515 | 0.75732598 | 4 | S2_S1 | 0.562785 | 0.50759598 | −0.055189021 | 0.53519049 |
| nsp8-P09132 | 140 | 150 | 120 | 0.78396 | 0.928905 | 0.916251186 | 2 | S2_S1_M | 0.144945 | 0.132291186 | −0.012653814 | 0.138618093 |
| nsp8-P10644 | 36.67 | 0 | 0 | 0.986265 | 0 | 0 | 5 | M | −0.986265 | −0.986265 | NA | −0.986265 |
| nsp8-P42285 | 60 | 30 | 23.33 | 0.87745 | 0.583995 | 0.607652812 | 2 | S2_S1_M | −0.293455 | −0.269797189 | 0.023657811 | −0.281626094 |
| nsp8-P51114 | 93.33 | 76.67 | 63.33 | 0.9278 | 0.6668 | 0.668238829 | 2 | S2_S1_M | −0.261 | −0.259561171 | 0.001438829 | −0.260280586 |
| nsp8-P51116 | 90 | 96.67 | 93.33 | 0.87708 | 0.67988 | 0.686838818 | 2 | S2_S1_M | −0.1972 | −0.190241183 | 0.006958817 | −0.193720591 |
| nsp8-P61011 | 6.18 | 30 | 40 | 0.577605 | 0.6537 | 0.872792074 | 2 | S2_S1_M | 0.076095 | 0.295187074 | 0.219092074 | 0.185641037 |
| nsp8-P82663 | 23.33 | 13.33 | 46.67 | 0.775315 | 0.439465 | 0.91321856 | 7 | S2_M | −0.33585 | 0.13790356 | 0.47375356 | NA |
| nsp8-Q03701 | 196.67 | 166.67 | 266.67 | 0.85365 | 0.72293 | 0.760986525 | 2 | S2_S1_M | −0.13072 | −0.092663475 | 0.038056525 | −0.111691738 |
| nsp8-Q12788 | 93.33 | 82 | 53.33 | 0.87482 | 0.73317 | 0.690414065 | 2 | S2_S1_M | −0.14165 | −0.184405936 | −0.042755936 | −0.163027968 |
| nsp8-Q13206 | 66.67 | 73.33 | 56.67 | 0.878515 | 0.89008 | 0.877876797 | 2 | S2_S1_M | 0.011565 | −0.000638203 | −0.012203203 | 0.005463398 |
| nsp8-Q14146 | 40 | 33.33 | 20 | 0.941165 | 0.777745 | 0.333093372 | 1 | S1_M | −0.16342 | −0.608071628 | −0.444651628 | NA |
| nsp8-Q14692 | 56.67 | 60 | 46.67 | 0.84302 | 0.8672 | 0.80826186 | 2 | S2_S1_M | 0.02418 | −0.034758141 | −0.058938141 | −0.00528907 |
| nsp8-Q15269 | 36.67 | 46.67 | 30 | 0.87901 | 0.688805 | 0.479327319 | 1 | S1_M | −0.190205 | −0.399682682 | −0.209477682 | NA |
| nsp8-Q15397 | 183.33 | 226.67 | 163.33 | 0.8118 | 0.86082 | 0.813323307 | 2 | S2_S1_M | 0.04902 | 0.001523307 | −0.047496693 | 0.025271654 |
| nsp8-Q16531 | 36.67 | 40 | 63.33 | 0.95416 | 0.64357 | 0.664919889 | 2 | S2_S1_M | −0.31059 | −0.289240112 | 0.021349889 | −0.299915056 |
| nsp8-Q4G0J3 | 96.67 | 150 | 126.67 | 0.719595 | 0.89692 | 0.906239841 | 2 | S2_S1_M | 0.177325 | 0.186644841 | 0.00931984 | 0.181984921 |
| nsp8-Q76FK4 | 83.33 | 43.33 | 20 | 0.902575 | 0.816175 | 0.760221042 | 2 | S2_S1_M | −0.0864 | −0.142353959 | −0.055953959 | −0.114376979 |
| nsp8-Q7L2J0 | 76.67 | 130 | 103.33 | 0.718475 | 0.89101 | 0.895489059 | 2 | S2_S1_M | 0.172535 | 0.177014059 | 0.004479059 | 0.174774529 |
| nsp8-Q7Z4Q2 | 23.33 | 0 | 0 | 0.96868 | 0 | 0 | 5 | M | −0.96868 | −0.96868 | NA | −0.96868 |
| nsp8-Q8IX01 | 23.33 | 0 | 0 | 0.83277 | 0 | 0 | 5 | M | −0.83277 | −0.83277 | NA | −0.83277 |
| nsp8-Q8IY37 | 23.33 | 43.33 | 0 | 0.580735 | 0.99481 | 0 | 1 | S1_M | 0.414075 | −0.580735 | −0.99481 | NA |
| nsp8-Q8N5D0 | 126.67 | 3.33 | 20 | 0.99578 | 0.0077805 | 0.683891711 | 7 | S2_M | −0.9879995 | −0.31188829 | 0.676111211 | NA |
| nsp8-Q8N983 | 0 | 23.33 | 0 | 0 | 0.98039 | 0 | 3 | S1 | 0.98039 | NA | −0.98039 | NA |
| nsp8-Q8NEJ9 | 16.67 | 33.33 | 36.67 | 0.603725 | 0.810405 | 0.85703947 | 2 | S2_S1_M | 0.20668 | 0.25331447 | 0.04663447 | 0.229997235 |
| nsp8-Q8NI36 | 50 | 63.33 | 83.33 | 0.879955 | 0.712755 | 0.73693436 | 2 | S2_S1_M | −0.1672 | −0.14302064 | 0.02417936 | −0.15511032 |
| nsp8-Q8TC07 | 43.33 | 0 | 0 | 0.99287 | 0 | 0 | 5 | M | −0.99287 | −0.99287 | NA | −0.99287 |
| nsp8-Q96B26 | 20 | 30 | 36.67 | 0.5721 | 0.97933 | 0.995449113 | 2 | S2_S1_M | 0.40723 | 0.423349113 | 0.016119113 | 0.415289556 |
| nsp8-Q96FK6 | 30 | 30 | 0 | 0.841435 | 0.991765 | 0 | 1 | S1_M | 0.15033 | −0.841435 | −0.991765 | NA |
| nsp8-Q96I59 | 13.33 | 3.33 | 53.33 | 0.750075 | 0.033522 | 0.890925175 | 7 | S2_M | −0.716553 | 0.140850175 | 0.857403175 | NA |
| nsp8-Q99547 | 20 | 23.33 | 13.33 | 0.84781 | 0.62049 | 0.647145842 | 2 | S2_S1_M | −0.22732 | −0.200664159 | 0.026655842 | −0.213992079 |
| nsp8-Q9BSC4 | 70 | 103.33 | 83.33 | 0.95159 | 0.900105 | 0.903909756 | 2 | S2_S1_M | −0.051485 | −0.047680245 | 0.003804755 | −0.049582622 |
| nsp8-Q9GZL7 | 36.67 | 26.67 | 20 | 0.918495 | 0.793965 | 0.606449939 | 2 | S2_S1_M | −0.12453 | −0.312045062 | −0.187515062 | −0.218287531 |
| nsp8-Q9H6F5 | 23.33 | 24 | 43.33 | 0.60171 | 0.970285 | 0.868401831 | 2 | S2_S1_M | 0.368575 | 0.266691831 | −0.10188317 | 0.317633415 |
| nsp8-Q9H6R4 | 123.33 | 90 | 56.67 | 0.866245 | 0.6852 | 0.677648918 | 2 | S2_S1_M | −0.181045 | −0.188596083 | −0.007551083 | −0.184820541 |
| nsp8-Q9HD40 | 13.33 | 10 | 43.33 | 0.642 | 0.36176 | 0.904779624 | 7 | S2_M | −0.28024 | 0.262779624 | 0.543019624 | NA |
| nsp8-Q9NQT4 | 23.33 | 30 | 36.67 | 0.77041 | 0.815345 | 0.847145951 | 2 | S2_S1_M | 0.044935 | 0.07673595 | 0.031800951 | 0.060835475 |
| nsp8-Q9NQT5 | 23.33 | 30 | 63.33 | 0.76155 | 0.791265 | 0.88739866 | 2 | S2_S1_M | 0.029715 | 0.12584866 | 0.09613366 | 0.07778183 |
| nsp8-Q9NTK5 | 46.67 | 3.33 | 46.67 | 0.78034 | 0.0067235 | 0.720728425 | 7 | S2_M | −0.7736165 | −0.059611576 | 0.714004925 | NA |
| nsp8-Q9NY61 | 23.33 | 70 | 116.67 | 0.803015 | 0.92578 | 0.891851841 | 2 | S2_S1_M | 0.122765 | 0.088836841 | −0.03392816 | 0.10580092 |
| nsp8-Q9UGI8 | 0 | 96.67 | 10 | 0 | 0.99523 | 0.507755438 | 4 | S2_S1 | 0.99523 | 0.507755438 | −0.487474562 | 0.751492719 |
| nsp8-Q9UHG3 | 86.67 | 0 | 0 | 0.995825 | 0 | 0 | 5 | M | −0.995825 | −0.995825 | NA | −0.995825 |
| nsp8-Q9UL40 | 2 | 33.33 | 0 | 0.20369 | 0.84735 | 0 | 3 | S1 | 0.64366 | NA | −0.84735 | NA |
| nsp8-Q9ULT8 | 0 | 53.33 | 53.33 | 0 | 0.913545 | 0.942752393 | 4 | S2_S1 | 0.913545 | 0.942752393 | 0.029207392 | 0.928148696 |
| nsp8-Q9ULX6 | 23.33 | 0 | 13.33 | 0.88436 | 0 | 0.42682183 | 5 | M | −0.88436 | −0.457538171 | NA | −0.670949085 |
| nsp8-Q9Y399 | 0 | 0 | 20 | 0 | 0 | 0.811028785 | 6 | S2 | NA | 0.811028785 | 0.811028785 | NA |
| nsp8-Q9Y3A4 | 30 | 10 | 13.33 | 0.881945 | 0.16819 | 0.330559314 | 5 | M | −0.713755 | −0.551385687 | NA | −0.632570343 |
| nsp9-O00142 | 0 | 96.67 | 73.33 | 0 | 0.992005 | 0.842759395 | 4 | S2_S1 | 0.992005 | 0.842759395 | −0.149245605 | 0.917382198 |
| nsp9-O00233 | 26.67 | 0 | 0 | 0.98034 | 0 | 0 | 5 | M | −0.98034 | −0.98034 | NA | −0.98034 |
| nsp9-P13984 | 0 | 23.2 | 140 | 0 | 0.777645 | 0.938713469 | 4 | S2_S1 | 0.777645 | 0.938713469 | 0.161068469 | 0.858179235 |
| nsp9-P21281 | 26.67 | 0 | 0 | 0.81161 | 0 | 0 | 5 | M | −0.81161 | −0.81161 | NA | −0.81161 |
| nsp9-P35555 | 0 | 6.67 | 153.33 | 0 | 0.502755 | 0.996186198 | 4 | S2_S1 | 0.502755 | 0.996186198 | 0.493431198 | 0.749470599 |
| nsp9-P35556 | 0 | 473.33 | 830 | 0 | 0.995555 | 0.995506165 | 4 | S2_S1 | 0.995555 | 0.995506165 | −4.88E−05 | 0.995530582 |
| nsp9-P35658 | 2 | 0 | 83.33 | 0.015781 | 0 | 0.981116632 | 6 | S2 | NA | 0.965335632 | 0.981116632 | NA |
| nsp9-P37198 | 0 | 3.33 | 180 | 0 | 0.082145 | 0.996505226 | 6 | S2 | NA | 0.996505226 | 0.914360226 | NA |
| nsp9-P38606 | 106.67 | 0 | 0 | 0.989065 | 0 | 0 | 5 | M | −0.989065 | −0.989065 | NA | −0.989065 |
| nsp9-P41250 | 20 | 0 | 0 | 0.927295 | 0 | 0 | 5 | M | −0.927295 | −0.927295 | NA | −0.927295 |
| nsp9-P49419 | 50 | 0 | 0 | 0.945525 | 0 | 0 | 5 | M | −0.945525 | −0.945525 | NA | −0.945525 |
| nsp9-P61962 | 0 | 50 | 160 | 0 | 0.880205 | 0.984617012 | 4 | S2_S1 | 0.880205 | 0.984617012 | 0.104412012 | 0.932411006 |
| nsp9-P62310 | 26.67 | 0 | 0 | 0.918185 | 0 | 0 | 5 | M | −0.918185 | −0.918185 | NA | −0.918185 |
| nsp9-Q14232 | 0 | 26.67 | 10 | 0 | 0.87989 | 0.496000682 | 4 | S2_S1 | 0.87989 | 0.496000682 | −0.383889318 | 0.687945341 |
| nsp9-Q15056 | 0 | 6.67 | 60 | 0 | 0.16176 | 0.934509695 | 6 | S2 | NA | 0.934509695 | 0.772749695 | NA |
| nsp9-Q5SW79 | 240 | 0 | 0 | 0.94098 | 0 | 0 | 5 | M | −0.94098 | −0.94098 | NA | −0.94098 |
| nsp9-Q6SZW1 | 26.67 | 0 | 0 | 0.74016 | 0 | 0 | 5 | M | −0.74016 | −0.74016 | NA | −0.74016 |
| nsp9-Q7Z3B4 | 0 | 0 | 213.33 | 0 | 0 | 0.995812411 | 6 | S2 | NA | 0.995812411 | 0.995812411 | NA |
| nsp9-Q86YT6 | 563.33 | 193.33 | 150 | 0.98055 | 0.857085 | 0.948911165 | 2 | S2_S1_M | −0.123465 | −0.031638835 | 0.091826165 | −0.077551918 |
| nsp9-Q8IWP9 | 50 | 6.67 | 0 | 0.96061 | 0.2048965 | 0 | 5 | M | −0.7557135 | −0.96061 | NA | −0.85816175 |
| nsp9-Q8N0X7 | 0 | 110 | 136.67 | 0 | 0.919655 | 0.981482065 | 4 | S2_S1 | 0.919655 | 0.981482065 | 0.061827065 | 0.950568532 |
| nsp9-Q8N1G2 | 10 | 30 | 0 | 0 | 0.689855 | 0 | 3 | S1 | 0.689855 | NA | −0.689855 | NA |
| nsp9-Q8TD19 | 10 | 56.67 | 390 | 0.697675 | 0.88751 | 0.995986433 | 2 | S2_S1_M | 0.189835 | 0.298311433 | 0.108476433 | 0.244073216 |
| nsp9-Q96F45 | 0.5 | 14.67 | 93.5 | 0.039492 | 0.7588 | 0.888790724 | 4 | S2_S1 | 0.719308 | 0.849298724 | 0.129990724 | 0.784303362 |
| nsp9-Q96PM5 | 63.33 | 0 | 0 | 0.90321 | 0 | 0 | 5 | M | −0.90321 | −0.90321 | NA | −0.90321 |
| nsp9-Q99567 | 0 | 0 | 36.67 | 0 | 0 | 0.95862156 | 6 | S2 | NA | 0.95862156 | 0.95862156 | NA |
| nsp9-Q9BU61 | 23.33 | 0 | 0 | 0.923145 | 0 | 0 | 5 | M | −0.923145 | −0.923145 | NA | −0.923145 |
| nsp9-Q9BVL2 | 0 | 0 | 120 | 0 | 0 | 0.989793112 | 6 | S2 | NA | 0.989793112 | 0.989793112 | NA |
| nsp9-Q9NZL9 | 0 | 0 | 43.33 | 0 | 0 | 0.989141328 | 6 | S2 | NA | 0.989141328 | 0.989141328 | NA |
| nsp9-Q9UBX5 | 10 | 0 | 20 | 0.496875 | 0 | 0.976001097 | 7 | S2_M | −0.496875 | 0.479126097 | 0.976001097 | NA |

TABLE 10B

| Column Headers | |
|---|---|
| from 8A | Description |
| Bait_Prey | Viral bait protein followed by uniprot identifier of |
| | human prey protein. |
| Bait | Viral bait protein. |
| Prey | Human prey protein as HGNC gene symbols. |
| MIST_MERS | MiST score for interaction in MERS-COV. |
| MIST_SARS1 | MiST score for interaction in SARS-COV-1. |
| MIST_SARS2 | MiST score for interaction in SARS-COV-2. |
| Saint_MERS | Saint score for interaction in MERS-COV. |
| Saint_SARS1 | Saint score for interaction in SARS-COV-1. |
| Saint_SARS2 | Saint score for interaction in SARS-COV-2. |
| BFDR_MERS | False discovery rate of Saint score for |
| | interaction in MERS-COV. |
| BFDR_SARS1 | False discovery rate of Saint score for |
| | interaction in SARS-COV-1. |
| BFDR_SARS2 | False discovery rate of Saint score for |
| | interaction in SARS-COV-2. |
| AvgSpec_MERS | Average spectral counts across three biological |
| | replicates for interaction in MERS-COV. |
| AvgSpec_SARS1 | Average spectral counts across three biological |
| | replicates for interaction in SARS-COV-1. |
| AvgSpec_SARS2 | Average spectral counts across three biological |
| | replicates for interaction in SARS-COV-2. |
| FoldChange_MERS | Fold change between spectral counts detected in |
| | experimental versus control samples for interaction |
| | in MERS-COV; derived from Saint scoring |
| | algorithm. |
| FoldChange_SARS1 | Fold change between spectral counts detected in |
| | experimental versus control samples for interaction |
| | in SARS-COV-1; derived from Saint scoring |
| | algorithm. |
| FoldChange_SARS2 | Fold change between spectral counts detected in |
| | experimental versus control samples for interaction |
| | in SARS-COV-2; derived from Saint scoring |
| | algorithm. |
| K_InteractionScore_ | Interaction score (K) for interaction from MERS- |
| MERS | COV, defined as the average between the MiST |
| | and Saint score. |
| K_InteractionScore_ | Interaction score (K) for interaction from SARS- |
| SARS1 | COV-1, defined as the average between the MiST |
| | and Saint score. |
| K_InteractionScore_ | Interaction score (K) for interaction from SARS- |
| SARS2 | COV-2, defined as the average between the MiST |
| | and Saint score. |
| Cluster | Cluster number assigned from hierarchical clustering. |
| Cluster_Assignments | Cluster category from hierarchical clusters. |
| | Annotations denote where interactions exist. |
| | M = MERS-COV only. |
| | S1 = SARS-COV-1 only. S2 = SARS-COV-2 only. |
| | S2_S1 = SARS-COV-2 and SARS-COV-1 only. |
| | S1_M = SARS-COV-1 and MERS-COV only. |
| | S2_M = SARS-COV-2 and MERS-COV only. |
| | S2_S1_M = SARS-COV-2, SARS-COV-1, and |
| | MERS-CoV. |
| DIS_SARS1_MERS | Differential interaction score comparing SARSI- |
| | MERS. Ranges from −1 to 1. DIS of 1 indicates |
| | SARS-COV-1 specificity, −1 indicates MERS- |
| | COV specificity, and 0 indicates shared between |
| | both. |
| DIS_SARS2_MERS | DIfferential interaction score comparing SARS2- |
| | MERS. Ranges from −1 to 1. DIS of 1 indicates |
| | SARS-COV-2 specificity, −1 indicates MERS-COV |
| | specificity, and 0 indicates shared between both. |
| DIS_SARS2_SARS1 | Differential interaction score comparing SARS2- |
| | SARS1. Ranges from −1 to 1. DIS of 1 indicates |
| | SARS-COV-2 specificity, −1 indicates SARS- |
| | COV-1 specificity, and 0 indicates shared between |
| | both. |
| DIS_SARS_MERS | Differential interaction score comparing SARS- |
| | MERS. Ranges from −1 to 1. DIS of 1 indicates |
| | SARS-COV-1 and SARS-COV-2 specificity, −1 |
| | indicates MERS-COV specificity, and 0 indicates |
| | shared between all three viruses. |

In agreement with previous results (FIG. 2A), DIS scores for the comparison between SARS-CoV-2 and SARS-CoV-1 are enriched near zero, indicating a high number of shared interactions (FIG. 15B, star). On the other hand, comparing interactions from either SARS-CoV-1 or SARS-CoV-2 with MERS-CoV resulted in DIS values closer to ±1, indicating a higher divergence (FIG. 15B, line and circle). The breakdown of DIS by homologous viral proteins reveals high similarity of interactions for proteins N, Nsp8, Nsp7, and Nsp13 (FIG. reinforcing the observations made by overlapping thresholded interactions (FIG. 15C and FIG. 15D). As the greatest dissimilarity was observed between the SARS-CoVs and MERS-CoV, a fourth DIS (SARS-MERS) was computed by averaging K from SARS-CoV-1 and SARS-CoV-2 prior to calculating the difference with MERS-CoV (FIG. 15B and FIG. triangle). Next, a network visualization of the SARS-MERS comparison was created (FIG. 15D), permitting an appreciation of SARS-specific (red; DIS near ±1) versus MERS-specific (blue; DIS near −1) interactions, as well as those conserved between all three coronavirus species (black; DIS near zero). SARS-specific interactions include: DNA polymerase a interacting with Nsp 1; stress granule regulators interacting with N protein; TLE transcription factors interacting with Nsp13; and AP2 clathrin interacting with Nsp10. Notable MERS-CoV-specific interactions include: mTOR and Stat3 interacting with Nsp1; DNA damage response components p53 (TP53), MRE11, RAD50, and UBR5 interacting with Nsp14; and the activating signal cointegrator 1 (ASC-1) complex interacting with Nsp2. Interactions shared between all three coronaviruses include: casein kinase II and RNA processing regulators interacting with N protein; IMP dehydrogenase 2 (IMPDH2) interacting with Nsp14; centrosome, protein kinase A, and TBK1 interacting with Nsp13; and the signal recognition particle, 7SK snRNP, exosome, and ribosome biogenesis components interacting with Nsp8 (FIG. 15D).

Referring to FIG. 15B, a density histogram of the DIS for all comparisons is shown.

Referring to FIG. 15C, a dot plot depicting the DIS of interactions from viral bait proteins shared between all three viruses, ordered left-to-right by the mean DIS per viral bait, is shown.

Referring to FIG. 15D, a virus-human protein-protein interaction map depicting the SARS-MERS comparison (triangle/purple in FIG. 15B-C) is shown. The network depicts interactions derived from cluster 2 (all 3 viruses), cluster 4 (SARS-CoV-1 and SARS-CoV-2), and cluster 5 (MERS-CoV only). Edge color denotes DIS: red, interactions specific to SARS-CoV-1 and SARS-CoV-2 but absent in MERS-CoV; blue, interactions specific to MERS-CoV but absent from both SARS-CoV-1 and SARS-CoV-2; black, interactions shared between all three viruses. Human-human interactions (thin dark grey line), proteins sharing the same protein complexes or biological processes (light yellow or light blue highlighting, respectively) are shown. Host-host physical interactions, protein complex definitions, and biological process groupings are derived from CORUM (M. Giurgiu, et al., CORUM: the comprehensive resource of mammalian protein complexes-2019. Nucleic Acids Res. 47, D559-D563 (2019)), Gene Ontology (biological process), and manually curated from literature sources. DIS=differential interactions score; SARS2=SARS-CoV-2; SARS1=SARS-CoV-1; MERS=MERS-CoV; SARS=both SARS-CoV-1 and SARS-CoV-2.

Cell-Based Genetic Screens Identify SARS-CoV-2 Host Dependency Factors

To identify host factors that are critical for infection and therefore potential targets for host-directed therapies, genetic perturbations of 332 human proteins were performed, 331 previously identified to interact with SARS-CoV-2 proteins (Gordon, et al. A SARS-CoV-2 protein interaction map reveals targets for drug repurposing. Nature (2020) plus ACE2, and their effect on infectivity observed. To ensure a broad coverage of potential hits, two screens in different cell lines were carried out to investigate the effects on infection: siRNA knockdowns in A549 cells stably expressing ACE2 (A549-ACE2) (FIG. 4A) and CRISPR-based knockouts in Caco-2 cells (FIG. 4B). ACE2 was included as positive control in both screens as were non-targeting siRNAs or non-targeted Caco-2 cells as negative controls. After SARS-CoV-2 infection, effects on virus infectivity were quantified by RT-qPCR on cell supernatants (siRNA) or by titrating virus-containing supernatants on Vero E6 cells (CRISPR). Cells were monitored for viability, and knockdown or editing efficiency was determined as described (FIG. 3A-F). This revealed that 93% of the genes were knocked down at least 50% in the A549-ACE2 screen, and 95% of the knockdowns exhibited less than a 20% decrease in viability. In the Caco-2 assay, an editing efficiency of at least 80% for 89% of the genes tested was observed (FIG. 3A-F). Of the 332 human SARS-CoV-2 interactors, the final A549-ACE2 dataset includes 331 gene knockdowns and the Caco-2 dataset includes 286 gene knockouts, with the difference mainly due to removal of essential genes. The readouts from both assays were then separately normalized using robust Z-scores, with negative and positive Z-scores indicating proviral dependency factors (perturbation=decreased infectivity) and antiviral host factors with restrictive activity (perturbation=increased infectivity), respectively. As expected, negative controls resulted in neutral Z-scores (FIG. 4C-D and Tables S6-7 provide in U.S. Provisional Application No. 63/091,929 filed on Oct. 15, 2020, expressly incorporated by reference herein). Similarly, perturbations of the positive control ACE2 resulted in strongly negative Z-scores in both assays (FIG. 4C-D). Overall, the Z-scores did not exhibit any trends related to viability, knockdown efficiency, or editing efficiency (FIG. 3A-F). With a cutoff of |Z|>2 to highlight genes that notably affect SARS-CoV-2 infectivity when perturbed, 31 and 40 dependency factors (Z<−2) and 3 and 4 factors with restrictive activity (Z>2) were identified in A549-ACE2 and Caco-2 cells, respectively (FIG. 4E). Of particular interest are the host dependency factors for SARS-CoV-2 infection, which represent potential targets for drug development and repurposing. For example, non-opioid receptor sigma 1 (sigma-1, encoded by SIGMAR1) was identified as a functional host-dependency factor in both cell systems in agreement with a previous report of antiviral activity for sigma receptor ligands (Gordon, et al. A SARS-CoV-2 protein interaction map reveals targets for drug repurposing. Nature (2020). To provide a contextual view of the genetics results, a network that integrates the hits from both cell lines and the PPIs of their encoded proteins with SARS-CoV-2, SARS-CoV-1, and MERS-CoV proteins was geneterated (FIG. 4F). Interestingly, an enrichment of genetic hits that encode proteins interacting with viral Nsp7, which has a high degree of interactions shared across all the three viruses, was observed (FIG. 2C). Prostaglandin E synthase 2 (encoded by PTGES2), for example, is a functional interactor of Nsp7 from SARS-CoV-1, SARS-CoV-2 and MERS-CoV. Other dependency factors were specific to SARS-CoV-2, including interleukin-17 receptor A (IL17RA), which interacts with SARS-CoV-2 Orf8. Dependency factors that are shared interactors between SARS-CoV-1 and SARS-CoV-2, such as the aforementioned sigma-1 (SIGMAR1) which interacts with Nsp6, and the mitochondrial import receptor subunit Tom70 (TOMM70) which interacts with Orf9b, were also identified.

SARS Orf9b Interacts with Tom70

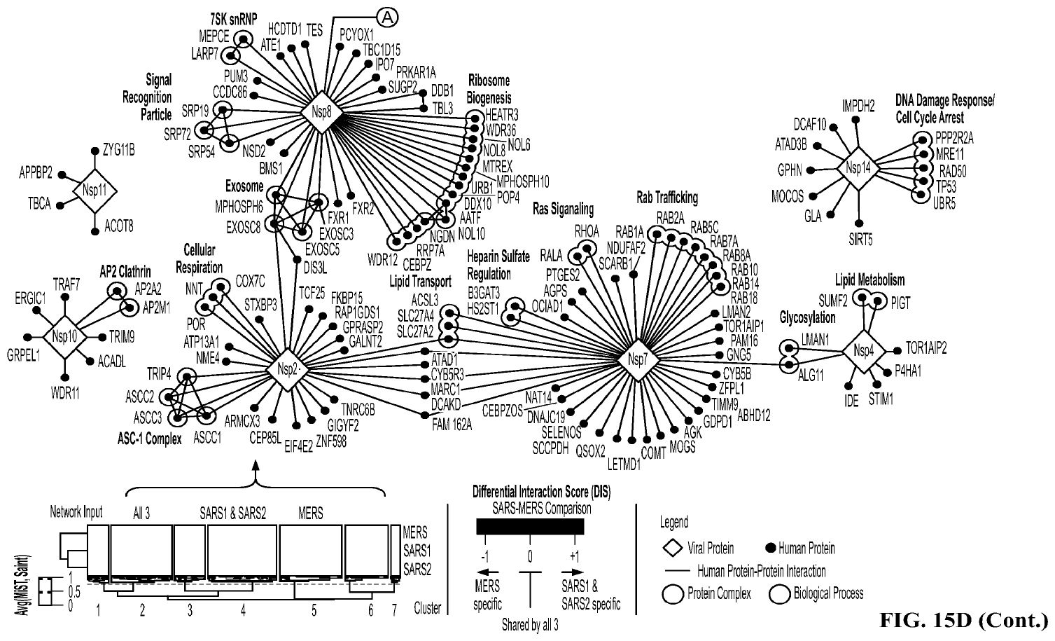

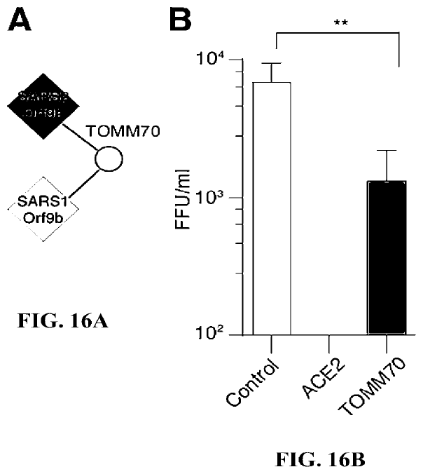

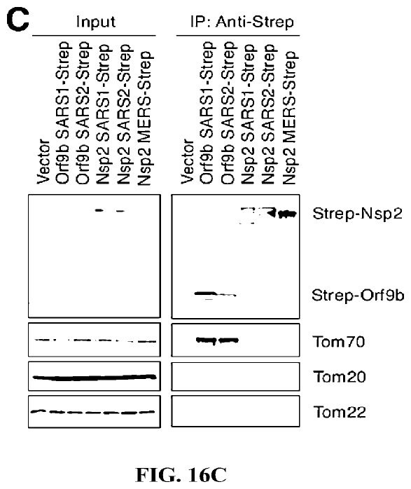

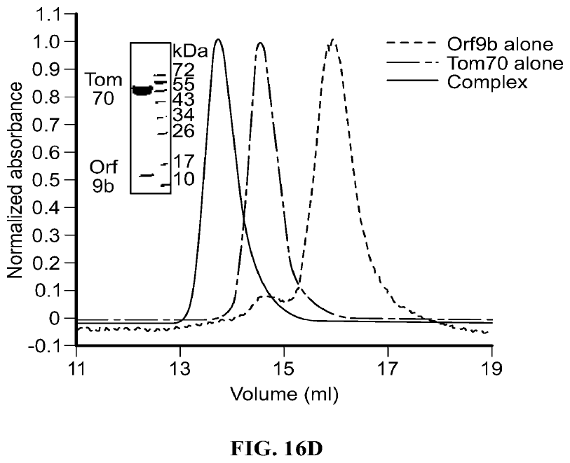

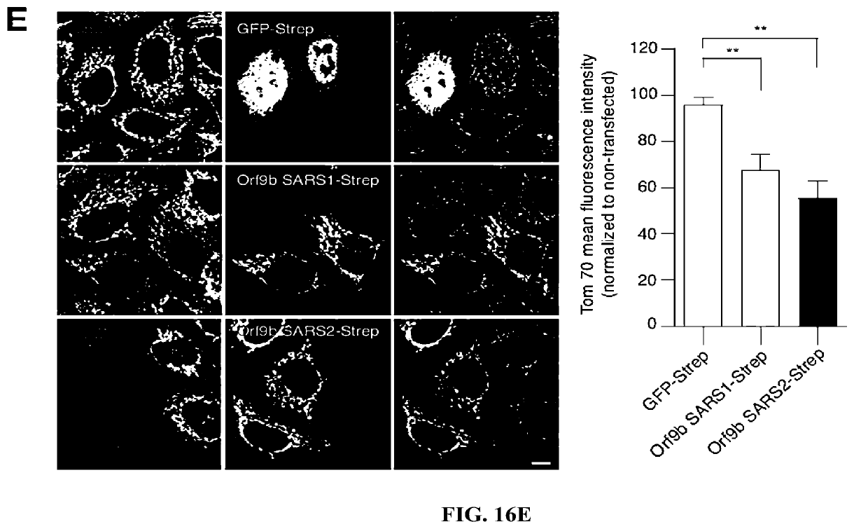

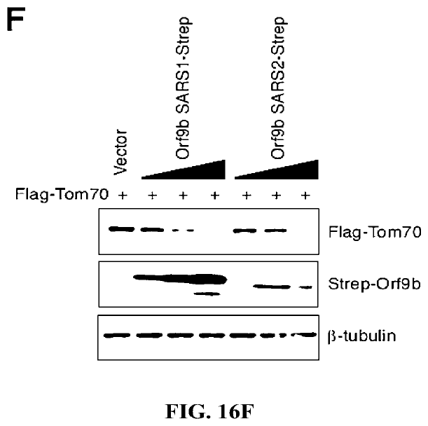

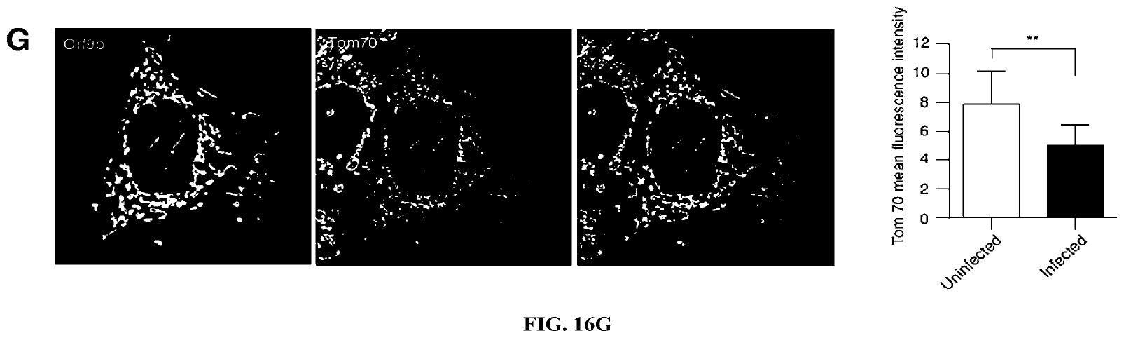

The mitochondrial outer membrane protein Tom70 (encoded by TOMM70) is a high-confidence interactor of Orf9b in both SARS-CoV-1 and SARS-CoV-2 interactomes (FIG. 16A) and a putative interactor of MERS-CoV Nsp2 with an observed interaction that falls below the scoring threshold. TOMM70 knockout in Caco-2 cells led to a significant decrease in viral titers upon SARS-CoV-2 infection, suggesting that Tom70 acts as a host dependency factor (FIG. 16B). Tom70 is one of the major import receptors in the TOM complex that recognizes and mediates the translocation of mitochondrial preproteins from the cytosol into the mitochondria in a chaperone dependent manner (J. C. Young, et al., Molecular chaperones Hsp90 and Hsp70 deliver preproteins to the mitochondrial import receptor Tom70. Cell. 112, 41-50 (2003)). Additionally, Tom70 is involved in the activation of MAVS-dependent antiviral signaling and apoptosis upon virus infection (R. Lin, et al., Tom70 imports antiviral immunity to the mitochondria. Cell Res. 20, 971-973 (2010); B. Wei, Tom70 mediates Sendai virus-induced apoptosis on mitochondria. J. Virol. 89, 3804-3818 (2015)).

Referring to FIG. 16A, Orf9b-Tom70 interaction is conserved between SARS-CoV-1 and SARS-CoV-2.

Referring to FIG. 16B, viral titers in Caco-2 cells after CRISPR knockout of TOMM70 or controls is shown.

Referring to FIG. 16C, co-immunoprecipitation of endogenous Tom70 with Strep-tagged Orf9b from SARS-CoV-1 and SARS-CoV-2, Nsp2 from SARS-CoV-1, SARS-CoV-2, and MERS-CoV, or vector control in HEK293T cells is shown. Representative blots of whole cell lysates and eluates after IP are shown.

Referring to FIG. 16D, size exclusion chromatography traces (10/300 S200 Increase) of Orf9b alone, Tom70 alone, and co-expressed Orf9b-Tom70 complex purified from recombinant expression in E. coli are shown. Insert shows SDS-PAGE of the complex peak indicating presence of both proteins.

Referring to FIG. 16E, immunostainings for Tom70 in HeLaM cells transfected with GFP-Strep and Orf9b from SARS-CoV-1 and SARS-CoV-2 (left) and mean fluorescence intensity±SD values of Tom70 in GFP-Strep and Orf9b expressing cells (normalized to nontransfected cells; right) are shown.

Referring to FIG. 16F, flag-Tom70 expression levels in total cell lysates of HEK293T cells upon titration of co-transfected Strep-Orf9b from SARS-CoV-1 and SARS-CoV-2 are shown.

Referring to FIG. 16G, immunostaining for Orf9b and Tom70 in Caco-2 cells infected with SARS-CoV-2 (left) and mean fluorescence intensity±SD values of Tom70 in uninfected and SARS-CoV-2 infected cells (right) is shown. SARS2=SARS-CoV-2; SARS1=SARS-CoV-1; MERS=MERS-CoV; IP=immunoprecipitation. **p<0.05. B, E, G, Student's t-test. E, scale bar=10 μm.

To validate the interaction between viral proteins and Tom70, a co-immunoprecipitation experiment was performed in the presence or absence of Strep-tagged Orf9b from SARS-CoV-1 and SARS-CoV-2 as well as Strep-tagged Nsp2 from all three CoVs. Endogenous Tom70, but not other translocase proteins of the outer membrane including Tom20, Tom22, and Tom40, co-precipitated only in the presence of Orf9b in both HEK293T and A549 cells, confirming the AP-MS data and suggesting that Orf9b specifically interacts with Tom70 (FIG. 16C and FIG. 17A). Further, upon co-expression in bacterial cells, it was possible to co-purify the Orf9b-Tom70 protein complex, indicating a high degree of stability (FIG. 16D). It was found that SARS-CoV-1 and SARS-CoV-2 Orf9b expressed in HeLaM cells co-localized with Tom70 (FIG. 16E), and it was observed that SARS-CoV-1 or SARS-CoV-2 Orf9b overexpression led to decreases in Tom70 expression (FIG. 16F). Similarly, Orf9b was found to co-localize with Tom70 upon SARS-CoV-2 infection (FIG. 16G). This is in agreement with the known outer mitochondrial membrane localization of Tom70 (A. M. Edmonson, et al., Characterization of a human import component of the mitochondrial outer membrane, TOMM70A. Cell Commun. Adhes. 9, 15-27 (2002)), and Orf9b localization to mitochondria upon over-expression and during SARS-CoV-2 infection (FIG. 6B). A decreases in Tom70 expression was also seen during SARS-CoV-2 infection (FIG. 16G) but did not see dramatic changes in expression levels of the mitochondrial protein Tom20 after individual Strep-Orf9b expression or upon SARS-CoV-2 infection (FIG. 17B-C).

Referring to FIG. 17A, co-immunoprecipitation between Strep-Orf9b and endogenous Tom70 is shown. A549 cells were transfected with Strep-tagged Orf9b from SARS-CoV-1 and SARS-CoV-2 along with Nsp2 from MERS-CoV. IP was performed using anti-Strep beads and representative immunoblots of whole cell lysates and eluates are shown.

Referring to FIG. 17B, immunostained images of SARS-CoV-2 Orf9b-expressing HeLaM cells stained for Tom20 and Strep-Orf9b (left) are shown. Mean fluorescence intensity±SD values of Tom20 in GFP-Strep and Orf9b expressing cells (normalized to non-transfected cells; right).

Referring to FIG. 17C, representative immunostained images of Orf9b and Tom20 upon SARS-CoV-2 infection are shown. IP=immunoprecipitation; SD=standard deviation.

CryoEM Structure of Orf9b-Tom70 Complex Reveals Orf9b Interacting at the Substrate Binding Site of Tom70

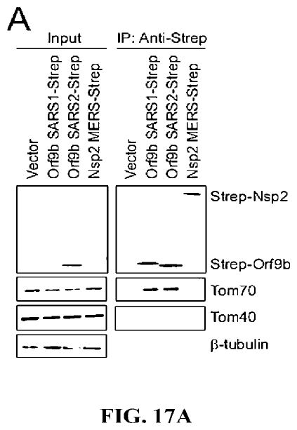

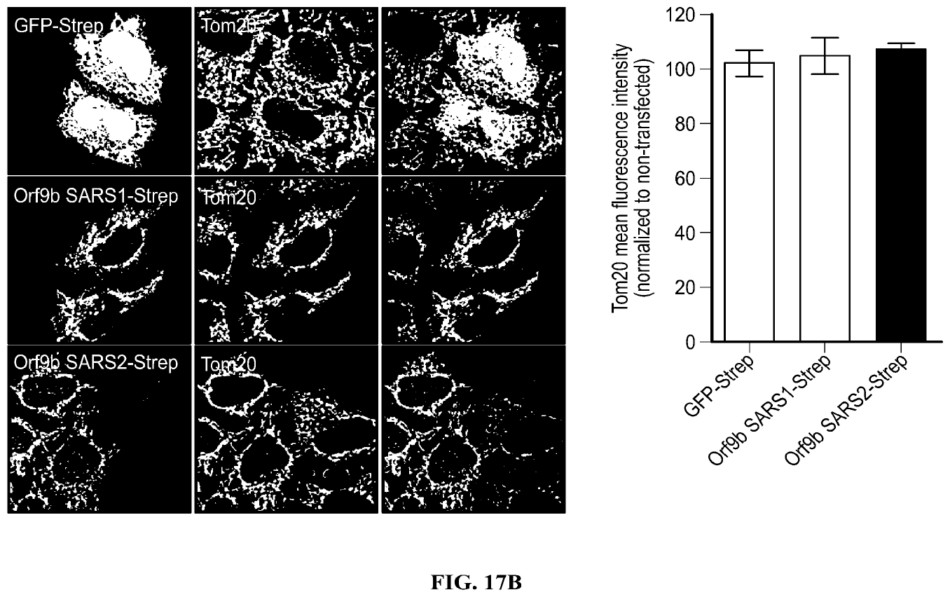

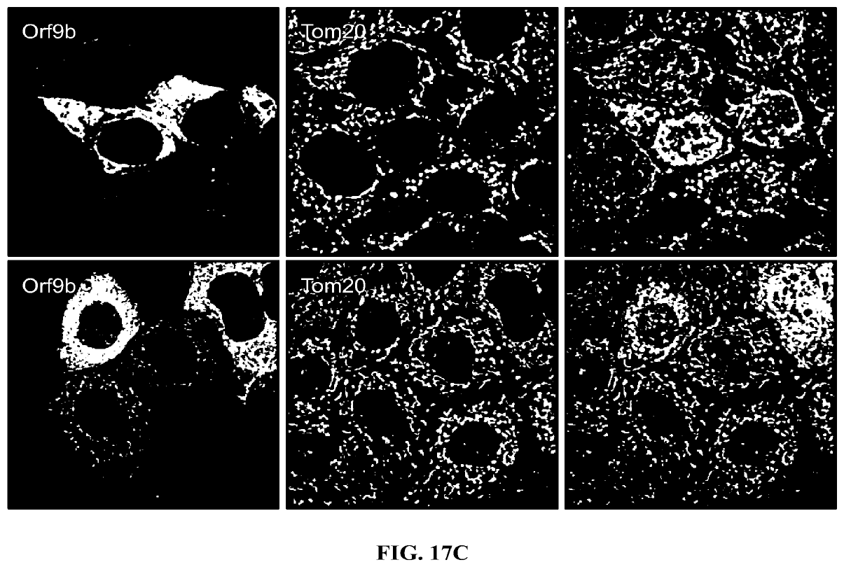

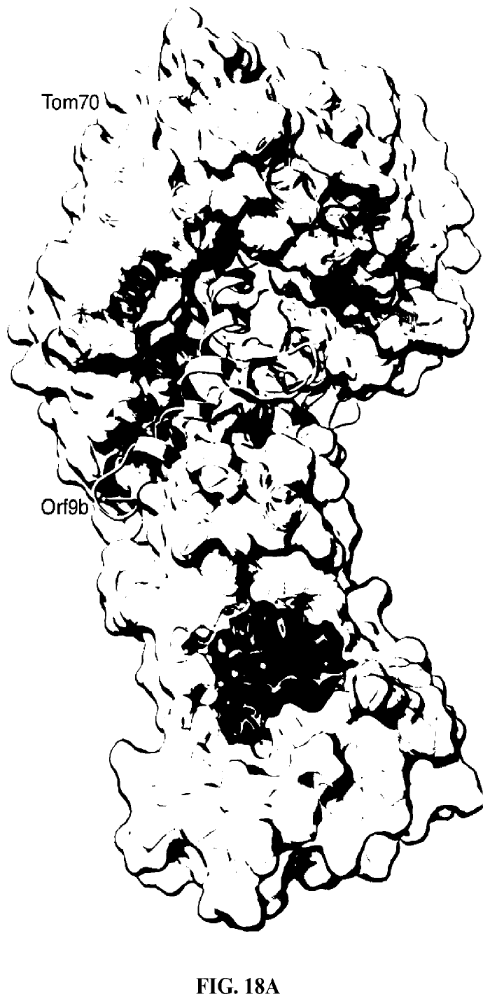

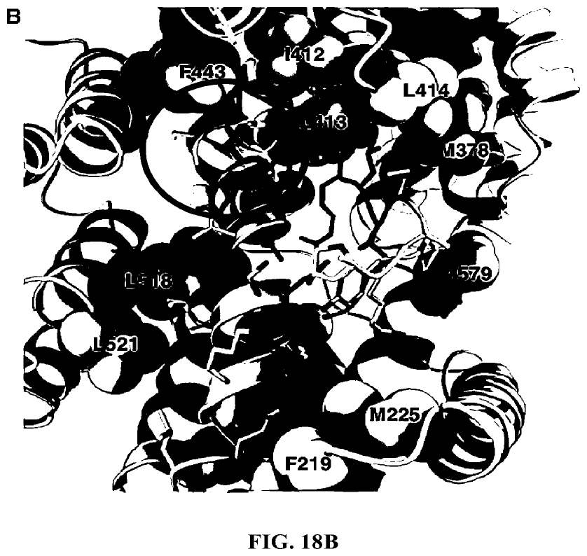

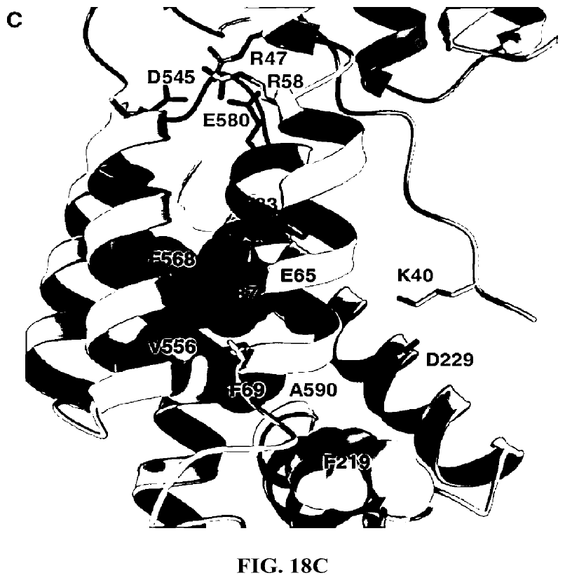

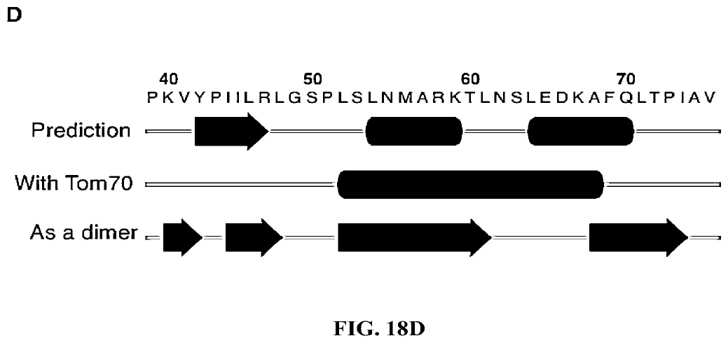

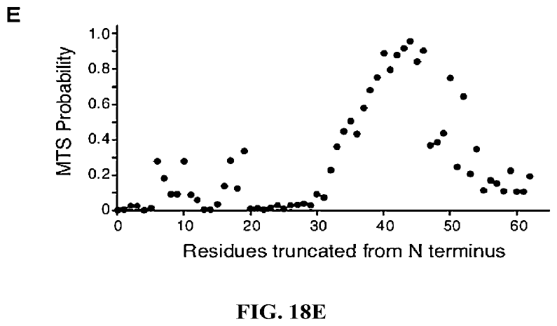

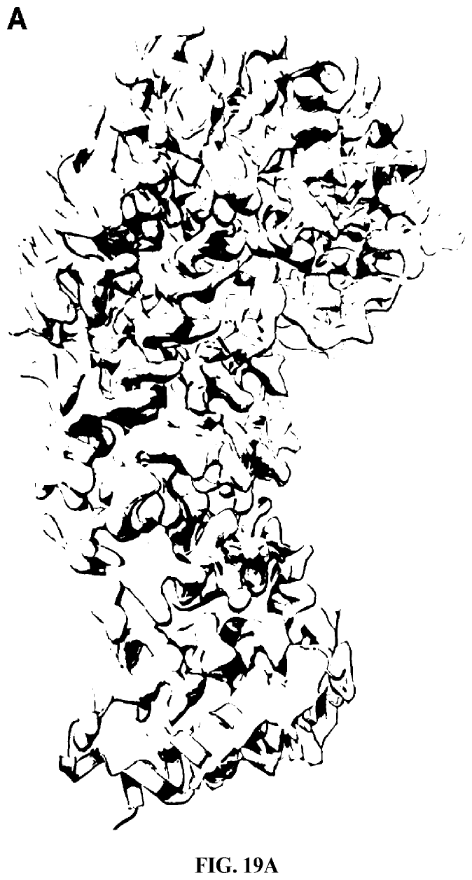

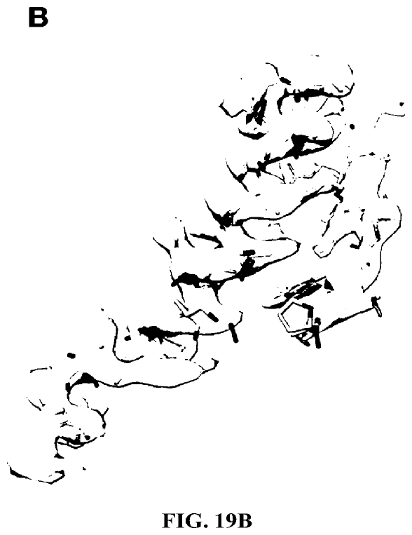

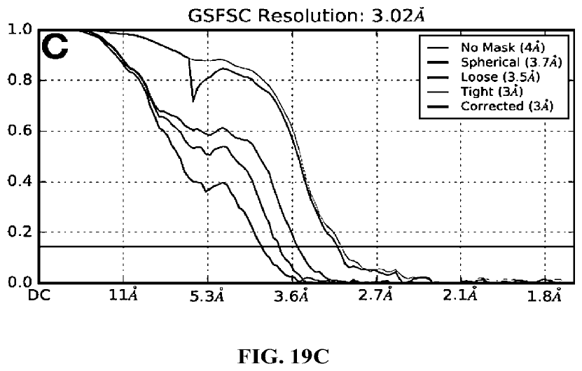

Tom70 preferentially binds preproteins with internal hydrophobic targeting sequences (J. Brix, et al., Differential recognition of preproteins by the purified cytosolic domains of the mitochondrial import receptors Tom20, Tom22, and Tom70. J Biol. Chem. 272, 20730-20735 (1997)). It contains an N-terminal transmembrane domain and tetratricopeptide repeat (TPR) motifs in its cytosolic segment. The C-terminal TPR motifs recognize the internal mitochondrial targeting signals (MTS) of preproteins, and the N-terminal TPR clamp domain serves as a docking site for multi-chaperone complexes that contain preprotein (J. Brix, et al., The mitochondrial import receptor Tom70: identification of a 25 kDa core domain with a specific binding site for preproteins. J. Mol. Biol. 303, 479-488 (2000); R. D. Mills, et al., Domain organization of the monomeric form of the Tom70 mitochondrial import receptor. J. Mol. Biol. 388, 1043-1058 (2009)). To further understand the molecular details of Orf9b-Tom70 interactions, a 3 Å cryoEM structure of the Orf9b-Tom70 complex was obtained (FIG. 18A and FIG. 19A-C). Interestingly, although purified proteins failed to interact upon attempted in vitro complex reconstitution, they yielded a stable and pure complex when co-expressed in E. coli (FIG. 16D). This may be due to the fact that Orf9b alone purifies as a dimer (as inferred by the apparent molecular weight on size exclusion chromatography) and would need to dissociate to interact with Tom70 based on the structure. Obtained cryoEM density allowed for atomic models to be built for residues 109-600 of human Tom70 and residues 39-76 of SARS-CoV-2 Orf9b (FIG. 18A and Table 11). Orf9b makes extensive hydrophobic interactions at the pocket on Tom70 that has been implicated in its binding to MTS, with the total buried surface area at the interface being quite extensive, approximately 2000 A² (FIG. 18B). In addition to the mostly hydrophobic interface, four salt bridges further stabilize the interaction (FIG. 18C). Upon interaction with Orf9b, the interacting helices on Tom70 move inward to tightly wrap around Orf9b as compared to previously crystallized yeast Tom70 homologs. No structure for human Tom70 without a substrate has been reported to date and therefore it cannot be ruled out that the conformational differences are due to differences between homologs. However, it is possible that this conformational change upon substrate binding is conserved across homologs as many of the Tom70 residues interacting with Orf9b are highly conserved, likely indicating residues essential for endogenous MTS substrate recognition.

Referring to FIG. 18A, a surface representation of the Orf9b-Tom70 structure. Tom70 is depicted as molecular surface in green, Orf9b is depicted as ribbon in orange. Region in charcoal indicates Hsp70/Hsp90 binding site on Tom70, is shown.

Referring to FIG. 18B, a magnified view of Orf9b-Tom70 interactions with interacting hydrophobic residues on Tom70 is indicated and shown in spheres. The two phosphorylation sites on Orf9b, S50 and S53, are shown in yellow.

Referring to FIG. 18C, ionic interactions between Tom70 and Orf9b are depicted as sticks. Highly conserved residues on Tom70 making hydrophobic interactions with Orf9b are depicted as spheres.

Referring to FIG. 19A, a cryoEM density (weighted by FSC and sharpened with a B-factor of −145) of Orf9b-Tom70 complex with the built atomic models depicted as ribbon is shown. Tom70 is in green, Orf9b is in orange.

Referring to FIG. 19B, a magnified view of the cryoEM density just around Orf9b indicated in sticks showing a good agreement between the density and the model is shown.

Referring to FIG. 19C, a gold standard Fourier shell correlation of the resulting reconstruction as output by cryosparc software package is shown.

TABLE 11

| | Orf9b-TOM70 |
|---|---|
| | (EMDB-XXXX) |
| | (PDB XXXX) |
| Data collection and processing | |
| Magnification | 105,000× |
| Voltage (kV) | 300 |
| Electron exposure (e−/Å²) | 66 |
| Dose rate (e−/pix/sec) | 8 |
| Defocus range (μm) | −0.7 to −2.4 |
| Pixel size (Å) | 0.834 (physical) |
| Symmetry imposed | C1 |
| Initial particle images (no.) | 2,805,121 |
| Final particle images (no.) | 178,373 |
| Map resolution (Å) | 3.05 |
| FSC threshold | 0.143 |
| Map resolution range (Å) | 3-4 |
| Refinement | |
| Initial model used (PDB code) | 3FP3 |
| Model resolution (Å) | 3.4 |
| FSC threshold | 0.5 |
| Model resolution range (Å) | 3-4 |
| Map sharpening B factor (Å2) | −145 |
| Model composition | |
| Non-hydrogen atoms | 4022 |
| Protein residues | 505 |
| Ligands | N/A |
| B factors (Å2) | |
| Protein | 60 |
| Ligand | N/A |
| R.m.s. deviations | |
| Bond lengths (Å) | 0.012 (1) |
| Bond angles (°) | 1.882 (3) |
| Validation | |
| MolProbity score | 0.55 |
| Clashscore | 0.12 |
| Poor rotamers (%) | 0.47 |
| Ramachandran plot | |
| Favored (%) | 0 |
| Allowed (%) | 1.4 |
| Disallowed (%) | 98.6 |

Surprisingly, although a previously published crystal structure of SARS-CoV-2 Orf9b revealed that it entirely consists of beta sheets (PDB:6Z4U) (S. D. Weeks, et al., X-ray Crystallographic Structure of Orf9b from SARS-CoV-2 (2020), doi:10.2210/pdb6z4u/pdb), upon binding Tom70 residues 52-68, Orf9b forms a helix (FIG. 18D). This is consistent with the fact that MTS sequences recognized by Tom70 are usually helical, and analysis with the TargetP MTS prediction server revealed a high probability for this region of Orf9b to possess an MTS (FIG. 18E). This shows an incredible structural plasticity in this viral protein where, depending on the binding partner, Orf9b changes between helical and beta strand folds. Furthermore, two infection-driven phosphorylation sites on Orf9b had been identified, S50 and S53 (M. Bouhaddou, et al., The Global Phosphorylation Landscape of SARS-CoV-2 Infection. Cell (2020)), which map to the region on Orf9b buried deep in the Tom70 binding pocket (FIG. 18B, within circle region). S53 contributes two hydrogen bonds to the interaction with Tom70 in this overall hydrophobic region. Therefore, once phosphorylated, it is likely that the Orf9b-Tom70 interaction is weakened. These residues are surface exposed in the dimeric structure of the Orf9b, which could potentially allow phosphorylation to partition Orf9b between Tom70-bound and dimeric populations.

Referring to FIG. 18D, a diagram depicting secondary structure comparison of Orf9b as predicted by Jpred server, as visualized in the structure herein, or as visualized in the previously-crystallized dimer structure (PDB:6Z4U) (S. D. Weeks, S. De Graef, A. Munawar, X-ray Crystallographic Structure of Orf9b from SARS-CoV-2 (2020), doi:10.2210/pdb6z4u/pdb) is shown. Pink tubes indicate helices, charcoal arrows indicate beta strands, amino acid sequence for the region visualized in the cryoEM structure is shown on top.

Referring to FIG. 18E, predicted probability of possessing an internal MTS as output by TargetP server by serially running N-terminally truncated regions of SARS-CoV-2 Orf9b. Region visualized in the cryoEM structure (amino acids 39-76) overlaps with the highest internal MTS probability region (amino acids 40-50) is shown. MTS=mitochondrial targeting signal.

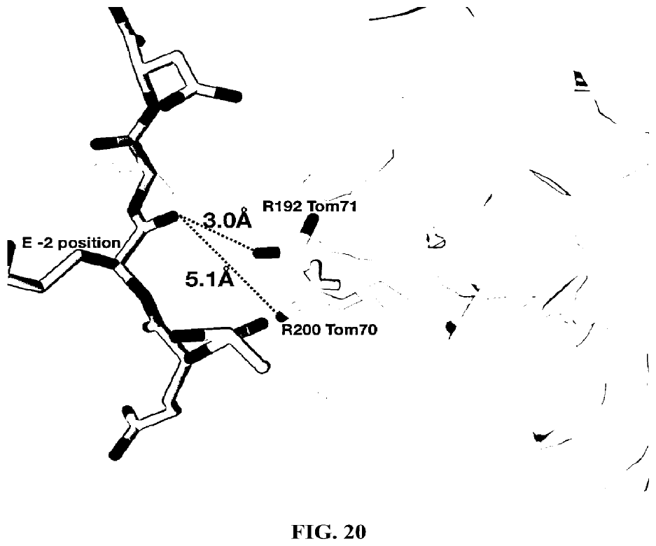

The two binding sites on Tom70—the substrate binding site and the TPR domain that recognizes Hsp70/Hsp90—are known to be conformationally coupled (M. Bouhaddou, et al., The Global Phosphorylation Landscape of SARS-CoV-2 Infection. Cell (2020)); J. Li, et al., Molecular chaperone Hsp70/Hsp90 prepares the mitochondrial outer membrane translocon receptor Tom71 for preprotein loading. J. Biol. Chem. 284, 23852-23859 (2009)). Tom70's interaction with a C-terminal EEVD motif of Hsp90 via the TPR domain is key for its function in the interferon pathway, and induction of apoptosis upon virus infection (B. Wei, et al., Tom70 mediates Sendai virus-induced apoptosis on mitochondria. J Virol. 89, 3804-3818 (2015); X.-Y. Liu, et al., Tom70 mediates activation of interferon regulatory factor 3 on mitochondria. Cell Res. 20, 994-1011 (2010)). It is hypothesized that Orf9b, by binding to the substrate recognition site of Tom70, allosterically inhibits Tom70's interaction with Hsp90 at the TPR domain. Indeed, it can be seen in the structure that R192, a key residue in the interaction with Hsp70/Hsp90, is moved out of position to interact with the EEVD sequence, suggesting that Orf9b may modulate interferon and apoptosis signaling via Tom70 (FIG. 20).

Referring to FIG. 20, a magnified view of R192/R200 (human Tom70/yeast Tom71), which is a key interacting residue with the EEVD motif from Hsp70/Hsp90, is shown. The conformation in yeast Tom71 (competent to bind EEVD, PDB:3FP2 (J. Li, X. Qian, J. Hu, B. Sha, Crystal structure of Tom71 complexed with Hsp82 C-terminal fragment (2009)) is shown in lavender. Conformation in our human Tom70 structure is shown in green, indicating that the arginine (R) is moved out of position to hydrogen bond with the glutamate. The EEVD peptide is shown as sticks in blue with the E at the −2 position (where terminal D is position 0) indicated. The cryoEM density is also shown depicting good agreement between the model and the density for R192.

Overall, the structure of Orf9b bound to Tom70 visualizes Orf9b in a completely different conformation than previously observed, potentially explaining the pleiotropic functions of this viral protein. In addition to being one of the smallest asymmetric protein complexes resolved at near-atomic resolution by cryoEM, it also clearly places Orf9b at a substrate binding site of Tom70, facilitating informed hypotheses on how Orf9b binding may regulate Tom70.

Implications of the Orf8-IL17RA Interaction for COVID-19

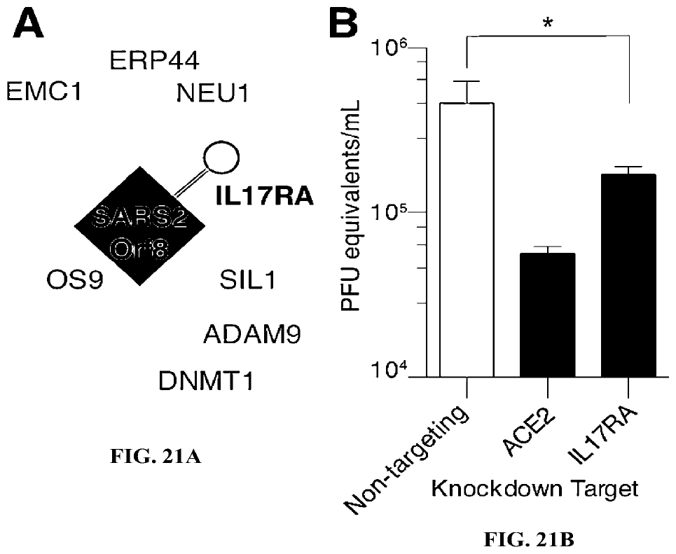

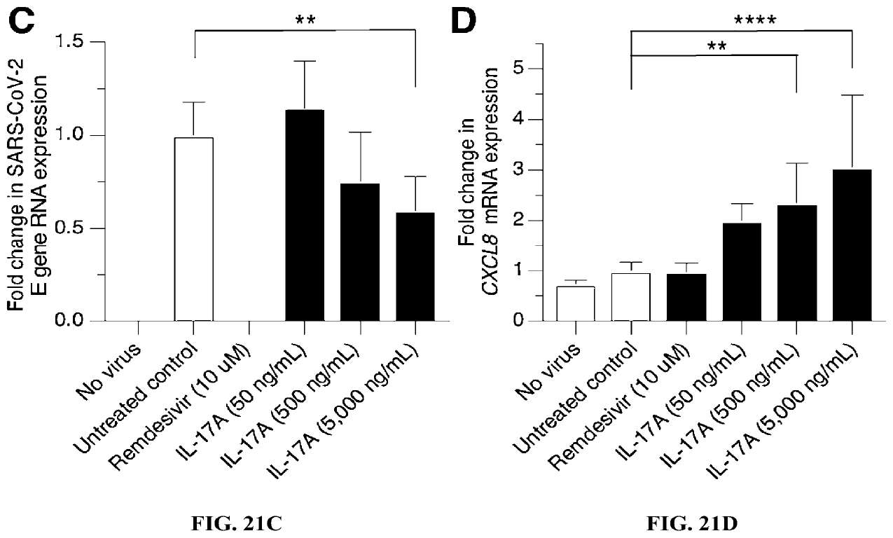

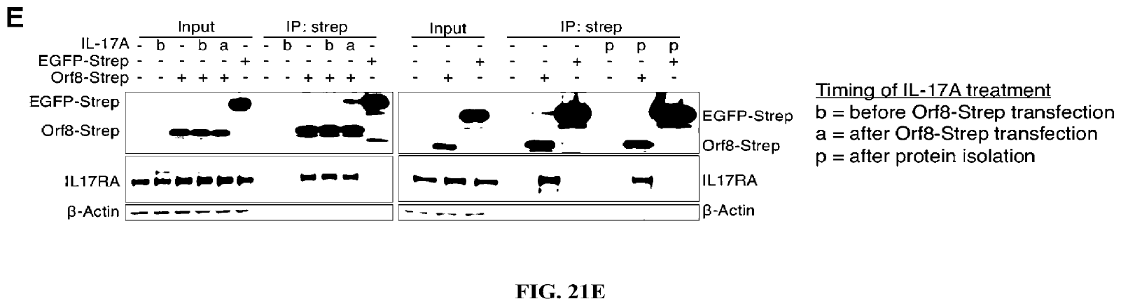

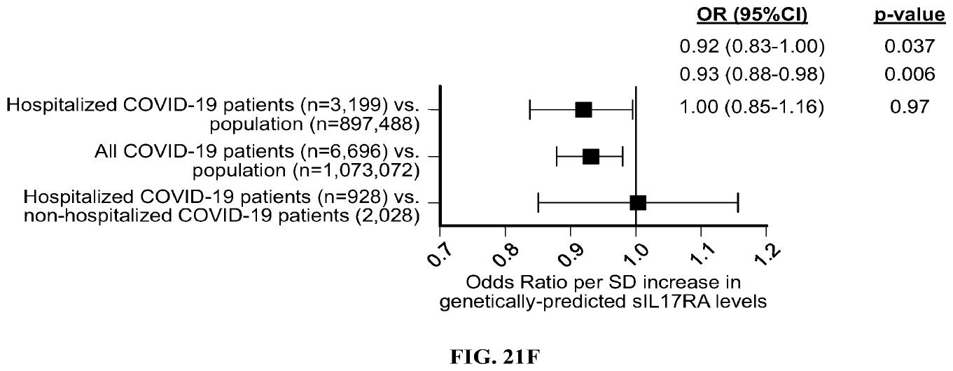

Infectious and transmissible SARS-CoV-2 viruses with large deletions of Orf8 have arisen during the pandemic and have been associated with milder disease and lower concentrations of pro-inflammatory cytokines (B. E. Young, et al., Effects of a major deletion in the SARS-CoV-2 genome on the severity of infection and the inflammatory response: an observational cohort study. Lancet. 396, 603-611 (2020)). Notably, compared to healthy controls, patients infected with wildtype but not Orf8-deleted virus had three-fold elevated plasma levels of IL-17A (B. E. Young, et al., Effects of a major deletion in the SARS-CoV-2 genome on the severity of infection and the inflammatory response: an observational cohort study. Lancet. 396, 603-611 (2020)). It was found that IL-17 receptor A (IL17RA) physically interacts with Orf8 from SARS-CoV-2, but not SARS-CoV-1 or MERS-CoV (FIG. 21A). Furthermore, knockdown of IL17RA or IL-17A treatment led to significant decreases in SARS-CoV-2 viral replication in A549-ACE2 cells (FIG. 21B-D). Regardless of whether IL-17A treatment occurred on cells before or after Orf8 plasmid transfection, or on bulk cell protein lysate, IL17RA was consistently and robustly found to immunoprecipitate with Orf8 in overexpression experiments, suggesting that IL-17A signaling or ligation to IL17RA does not disrupt the interaction with Orf8 (FIG. 21E).

Referring to FIG. 21A, IL17RA is a functional interactor of SARS-CoV-2 Orf8. Only interactors identified in the genetic screening are shown.

Referring to FIG. 21B, viral titers of after IL17RA or control knockdown in A549-ACE2 cells are shown.

Referring to FIG. 21C, viral gene E RNA expression after infection with indicated agents in A549-ACE2 cells is shown.

Referring to FIG. 21D, CXCL8 mRNA expression after infection with indicated agents in A549-ACE2 cells. Plots represent 2 biological replicates with 3 technical replicates each.

Referring to FIG. 21E, co-immunoprecipitation of endogenous IL17RA with Strep-tagged Orf8 or EGFP with or without IL-17A treatment at different times is shown. Overexpression was done in HEK293T cells.

Referring to FIG. 21F, odds ratio of membership in indicated cohorts by genetically-predicted sIL17RA levels. SARS2=SARS-CoV-2; IP=immunoprecipitation; SD=standard deviation; OR=odds ratio; CI=confidence interval; sIL17RA=soluble IL17RA. *=p<0.05, **=p<0.005, ****=p<0.00005. B, unpaired t-test; C-D, one-way ANOVA relative to untreated control condition with Dunnet multiple comparison correction. Error bars in B-D indicate SD; in F they indicate 95% CI.

Orf8 may use its physical interaction with IL17RA to modulate IL-17 signaling systemically, which may not be readily detectable in in vitro epithelial cell monoculture experiments. One manner in which IL-17 signaling is regulated is through the release of the extracellular domain as soluble IL17RA (sIL17RA), which acts as a decoy receptor in circulation and inhibits IL-17 signalling (M. Zaretsky, et al., Directed evolution of a soluble human IL-17A receptor for the inhibition of psoriasis plaque formation in a mouse model. Chem. Biol. 20, 202-211 (2013)). Production of sIL17RA has been demonstrated by alternative splicing in cultured cells (Identification of a soluble isoform of human IL-17RA generated by alternative splicing. Cytokine. 64, 642-645 (2013)), but the mechanism by which IL17RA is shed in vivo remains unclear (Biological functions and therapeutic opportunities of soluble cytokine receptors. Cytokine Growth Factor Rev. (2020)). ADAM family proteases—including dependency factor ADAM9—are known to mediate the release of other interleukin receptors into their soluble form (M. Sammel, et al., Differences in Shedding of the Interleukin-11 Receptor by the Proteases ADAM9, ADAM10, ADAM17, Meprin α, Meprin β and MT1-MMP. Int. J. Mol. Sci. 20, 3677 (2019)). Interestingly, it was found that SARS-CoV-2 Orf8 interacted with both ADAM9 and ADAMTS1 in a previous study (D. E. Gordon, et al. Nature (2020)). In order to test the in vivo relevance of sIL17RA in modulating SARS-CoV-2 infection, the largest proteomic genome-wide association study (GWAS) to date was used, which identified 14 single nucleotide polymorphisms (SNPs) near the IL17RA gene that causally regulate sIL17RA plasma levels (B. B. Sun, Jet al., Genomic atlas of the human plasma proteome. Nature. 558, 73-79 (2018)). Then, generalized summary-based Mendelian randomization (GSMR) was used (B. B. Sun, Jet al., Genomic atlas of the human plasma proteome. Nature. 558; Z. Zhu, et al., Causal associations between risk factors and common diseases inferred from GWAS summary data. Nat. Commun. 9, 224 (2018)) on the curated GWAS datasets of the COVID-19 Host Genetics Initiative (COVID-HGI) (C. Huang, et al., The COVID-19 Host Genetics Initiative, a global initiative to elucidate the role of host genetic factors in susceptibility and severity of the SARS-CoV-2 virus pandemic. Eur. J Hum. Genet. 28, 715-718 (2020)) and it was observed that increased predicted sIL17RA plasma levels were associated with lower risk of COVID-19 when compared to the population (FIG. 21F and Table 12A-B). Similar results were obtained when comparing only hospitalized COVID-19 patients to the population. However, there was no evidence of association in hospitalized versus non-hospitalized COVID-19 patients. Though the COVID-HGI dataset is underpowered and this observation needs to be replicated in other cohorts, the evidence suggests that genetically-predicted higher sIL17RA levels may be associated with disease susceptibility, but not necessarily disease severity amongst symptomatic individuals. Overall, this is consistent with the improved clinical outlook for infections with Orf8-deleted virus.

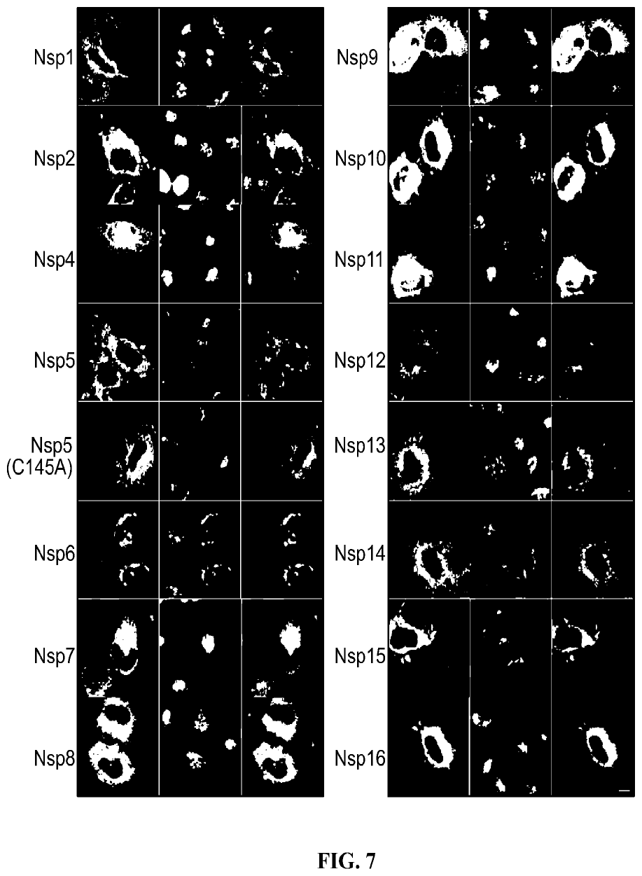

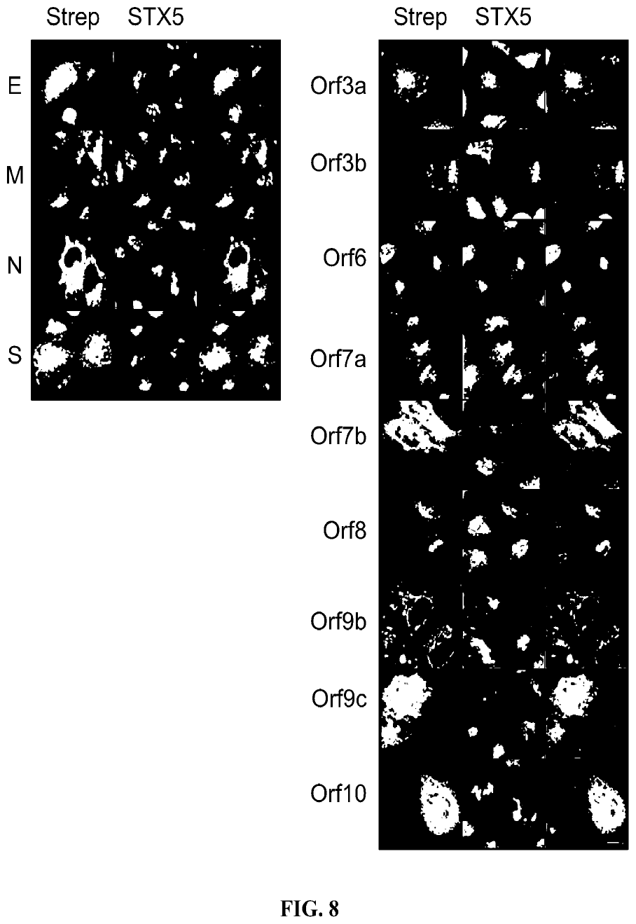

TABLE 12A

| Column | Definition |
|---|---|
| Comparison | Indication of which comparison in FIG. 8F is |
| | being described |
| Case | Phenotype definition of case as established in COVID-HGI |
| definition | “Phenotype defnitions for analyses v 2.0” found here: |
| | https://docs.google.com/document/d/ |
| | 1okamrqYmJfa35ClLvCt_vEe4PkvrTwggHq7T3jbeyCI/edit |
| Case n | Number of individuals in the case cohort |
| Control | Phenotype definition of case as established in COVID-HGI |
| definition | “Phenotype defnitions for analyses v 2.0” found here: |
| | https://docs.google.com/document/d/ |
| | 1okamrqYmJfa35ClLvCt_vEe4PkvrTwggHq7T3jbeyCI/edit |
| Control n | Number of individuals in the control cohort |
| n SNPs | number of cis-acting IL17RA pQTL SNPs analyzed |
| p | p value of comparison |
| OR | Odds ratio of comparison |
| LCI | Lower bound of the 95% confidence interval |
| UCI | Upper bound of the 95% confidence interval |

TABLE 12B

| | Case | Case | Control | Control | | | | | |
|---|---|---|---|---|---|---|---|---|---|
| Comparison | definition | n | definition | n | nSNPs | p | OR | LCI | UCI |
| hospitalized_covid_vs_pop- | Hospitalized | 3199 | Everybody | 897488 | 12 | 0.0371043 | 0.92008134 | 0.85077536 | 0.99503313 |
| ulation | laboratory | | that is | | | | | | |
| | confirmed | | not a case, | | | | | | |
| | SARS-CoV- | | e.g. | | | | | | |
| | 2 infection | | population | | | | | | |
| | (RNA and/or | | | | | | | | |
| | serology | | | | | | | | |
| | based) OR | | | | | | | | |
| | hospitalization | | | | | | | | |
| | due to corona- | | | | | | | | |
| | related | | | | | | | | |
| | symptoms. | | | | | | | | |
| covid_vs_population | Individuals | 6696 | Everybody | 1073072 | 14 | 0.00586206 | 0.93156836 | 0.88576034 | 0.97974539 |
| | with laboratory | | that is | | | | | | |
| | confirmation | | not a case, | | | | | | |
| | of SARS-CoV- | | e.g. | | | | | | |
| | 2 infection | | population | | | | | | |
| | (RNA and/or | | | | | | | | |
| | serology | | | | | | | | |
| | based) OR | | | | | | | | |
| | EHR/ICD | | | | | | | | |
| | coding/ | | | | | | | | |
| | Physician | | | | | | | | |
| | Confirmed | | | | | | | | |
| | COVID-19 OR | | | | | | | | |
| | self-reported | | | | | | | | |
| | COVID-19 | | | | | | | | |
| | positive | | | | | | | | |
| | (e.g. by | | | | | | | | |
| | questionnaire) | | | | | | | | |
| hospital- | Hospitalized | 928 | Laboratory | 2028 | 13 | 0.965391 | 1.003398 | 0.86084471 | 1.16955768 |
| ized_covid_vs_not_hos- | laboratory | | confirmed | | | | | | |
| pitalized_covid | confirmed | | SARS-CoV- | | | | | | |
| | SARS-CoV- | | 2 infection | | | | | | |
| | 2 infection | | (RNA and/or | | | | | | |
| | (RNA and/or | | serology | | | | | | |
| | serology | | based) | | | | | | |
| | based) OR | | AND not | | | | | | |
| | hospitalization | | hospitalised | | | | | | |
| | due to corona- | | 21 days after | | | | | | |
| | related | | the test. | | | | | | |
| | symptoms. | | | | | | | | |

Investigation of Druggable Targets Identified as Interactors of Multiple Coronaviruses

The identification of druggable host factors provides a rationale for drug repurposing efforts. Given the extent of the current pandemic, real-world data can now be used to study the outcome of COVID-19 patients coincidentally treated with host factor-directed, FDA-approved therapeutics. Using medical billing data, 738,933 patients in the United States with documented SARS-CoV-2 infection were identified. In this cohort, the use of drugs against targets identified here that were shared across coronavirus strains was probed, and found to be functionally relevant in the genetic perturbation screens. In particular, outcomes for an inhibitor of prostaglandin E synthase type 2 (PGES-2, encoded by PTGES2) and for ligands of sigma non-opioid receptor 1 (sigma-1, encoded by SIGMAR1) were analyzed, and whether these patients fared better than carefully-matched patients treated with clinically-similar drugs that do not act on coronavirus host factors was investigated.

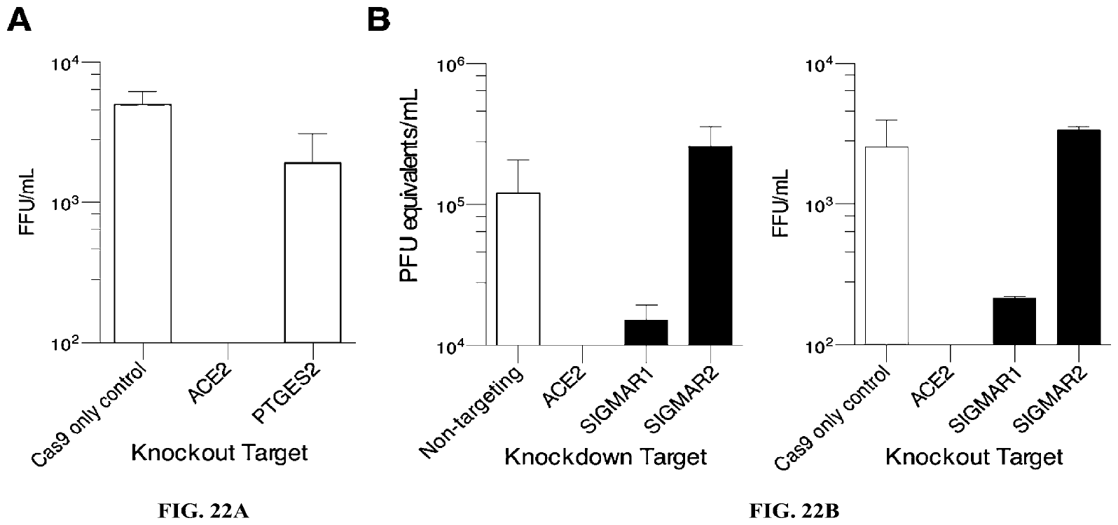

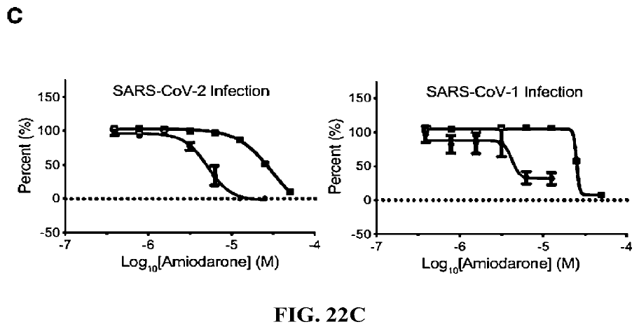

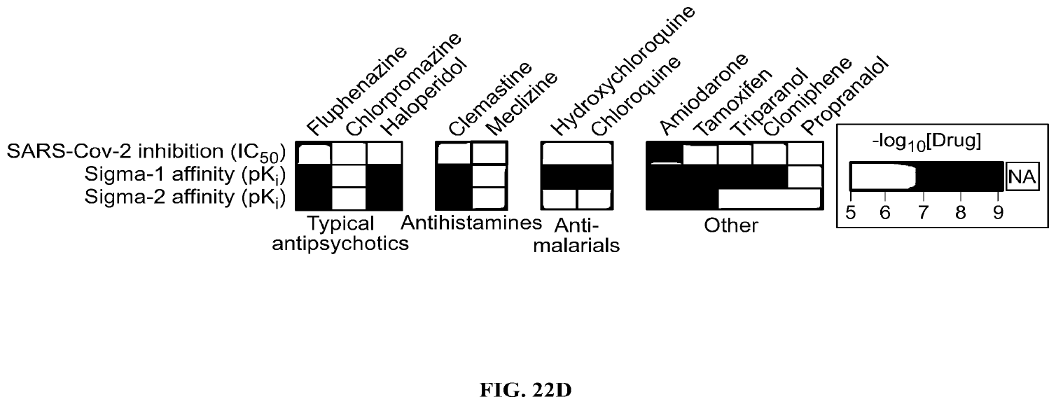

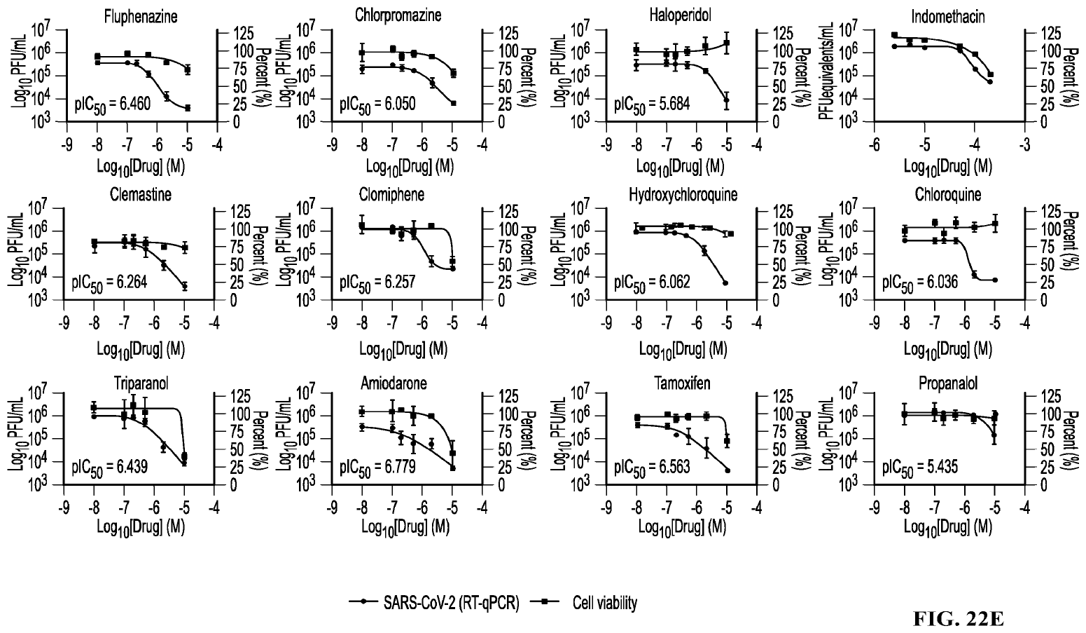

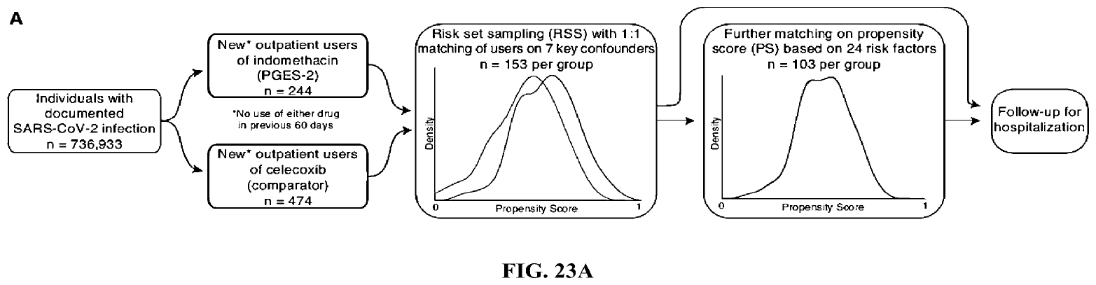

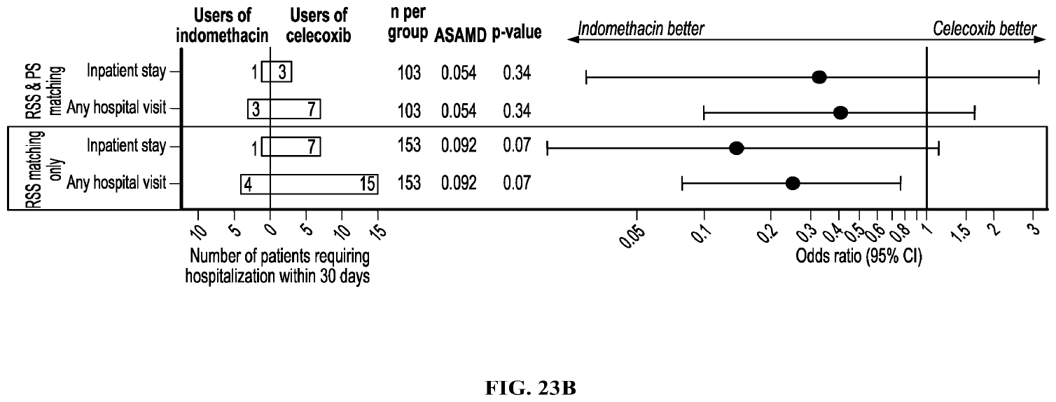

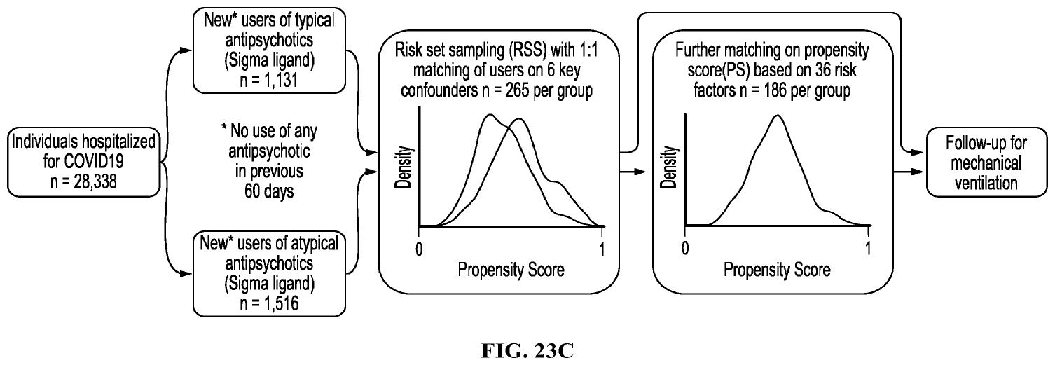

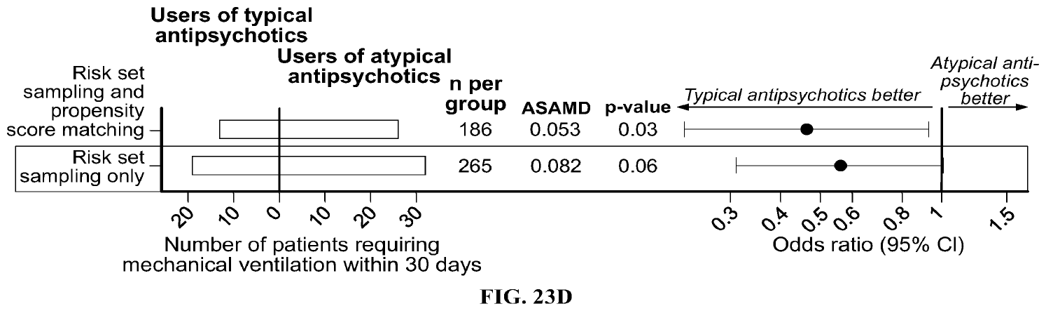

PGES-2, an interactor of Nsp7 from all three viruses (FIG. 15D), is a dependency factor for SARS-CoV-2 (FIG. 4F). It is inhibited by the FDA-approved prescription nonsteroidal anti-inflammatory drug (NSAID) indomethacin. Computational docking of Nsp7 and PGES-2 to predict binding configuration showed that the dominant cluster of models localizes Nsp7 adjacent to the PGES-2-indomethacin binding site (FIG. 20A-C). However, indomethacin did not inhibit SARS-CoV-2 in vitro at reasonable antiviral concentrations (FIG. 22A-E). A previous study also found that similarly high levels of the drug were needed for inhibition of SARS-CoV-1 in vitro, but still showed efficacy for indomethacin against canine coronavirus in vivo (C. Amici, et al., Indomethacin has a potent antiviral activity against SARS coronavirus. Antivir. Ther. 11, 1021-1030 (2006)). This provided motivation to observe outcomes in a cohort of outpatients with confirmed SARS-CoV-2 infection who by happenstance initiated a course of indomethacin, as compared to those who initiated the prescription NSAID celecoxib, which lacks anti-PGES-2 activity. The odds of hospitalization were compared by risk-set sampling (RSS) patients treated at the same time and at similar levels of disease severity and then further matching on propensity score (PS) (P. R. Rosenbaum, D. B. Rubin, The central role of the propensity score in observational studies for causal effects. Biometrika. 70, 41-55 (1983)) (FIG. 23A and Table 7A-I). This new user, active comparator design mimics the interventional component of prospective clinical studies. Relative to celecoxib, indomethacin treatment showed a strong trend towards improved outcomes (FIG. 23B). In sensitivity analysis, neither using the larger, risk-set-sampled cohort nor relaxing the outcome definition to include any hospital visit appreciably changed the trend that was initially observed, but it did increase the significance of the observation: SARS-CoV-2-positive, new users of indomethacin in the outpatient setting were less likely than matched new users of celecoxib to require hospitalization or inpatient services. While it is important to acknowledge that this is a small, non-interventional study, it is nonetheless a powerful example of how molecular insight can rapidly generate testable clinical hypotheses and help prioritize candidates for prospective clinical trials or future drug development.

Referring to FIG. 22A, SARS-CoV-2 replication in Caco-2 cells after knockout of PTGES2 or controls is shown.

Referring to FIG. 22B, SARS-CoV-2 replication in A549-ACE2 cells or Caco-2 cells after knockdown and knockout, respectively, of SIGMAR1, SIGMAR2 (TMEM97) or controls is shown.

Referring to FIG. 22C, antiviral activity of amiodarone against SARS-CoV-2 (left) and SARS-CoV-1 (right) in Vero E6 cells is shown.

Referring to FIG. 22D, clinically-approved sigma receptor-targeting drugs with verified anti-SARS-CoV-2 activity by clinical drug class are shown. Heatmap indicates, from top to bottom: pIC50 (−log 10[IC50]) of the drug against SARS-CoV-2; reported pKi (−log 10[Ki]) of the drug against sigma-1 receptor; reported pKi of the drug against sigma-2 receptor. SARS-CoV-2 IC50 was determined in A549-ACE2 cells or in Vero E6 cells where indicated by a black border. Grey boxes indicate no value was reported in the literature.

Referring to FIG. 22E, performance of representative clinical drugs against SARS-CoV-2 in vitro in A549-ACE2 cells is shown. Error bars indicate standard deviation.

Referring to FIG. 23A, a schematic of retrospective real-world clinical data analysis of indomethacin use for outpatients with SARS-CoV-2 is shown. Plots show distribution of propensity scores for all included patients (red, indomethacin users; blue, celecoxib users). For a full list of inclusion, exclusion, and matching criteria see Table 7A-I.

Referring to FIG. 23B, the effectiveness of indomethacin vs. celecoxib in patients with confirmed SARS-CoV-2 infection treated in an outpatient setting is shown. Average standardized absolute mean difference (ASAMD) is a measure of balance between indomethacin and celecoxib groups calculated as the mean of the absolute standardized difference for each propensity score factor (Table 7A-I); p-value and odds ratios with 95% CI are estimated using the Aetion Evidence Platform r4.6. No ASAMD was greater than 0.1.

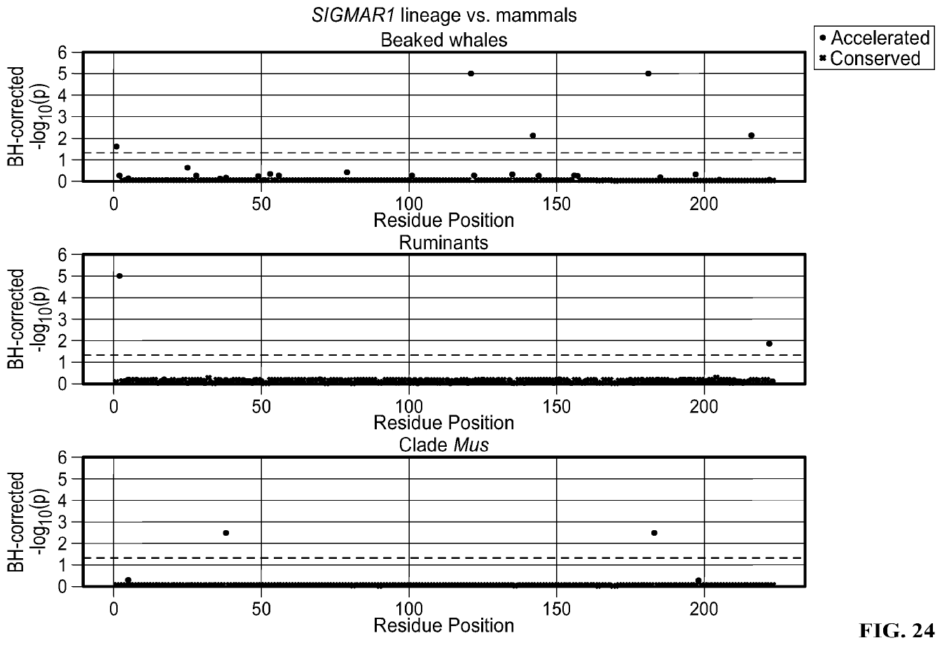

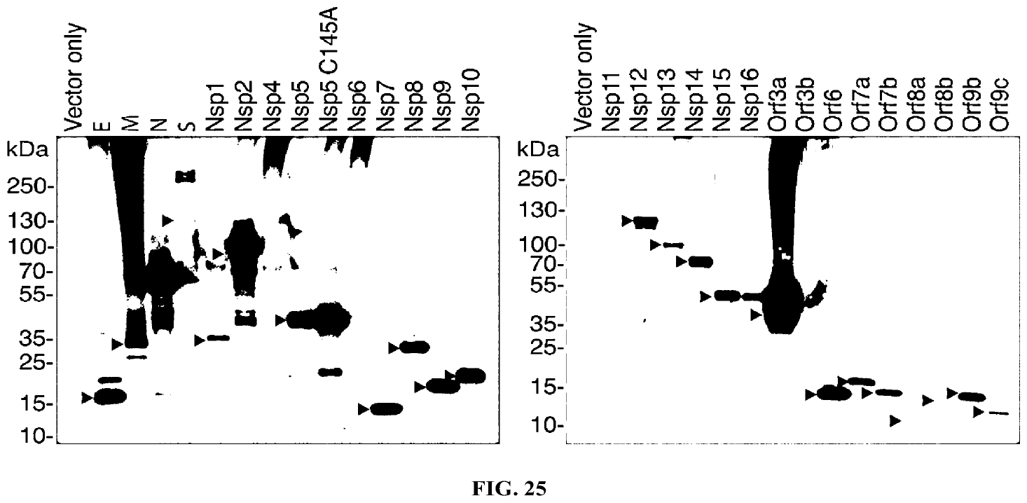

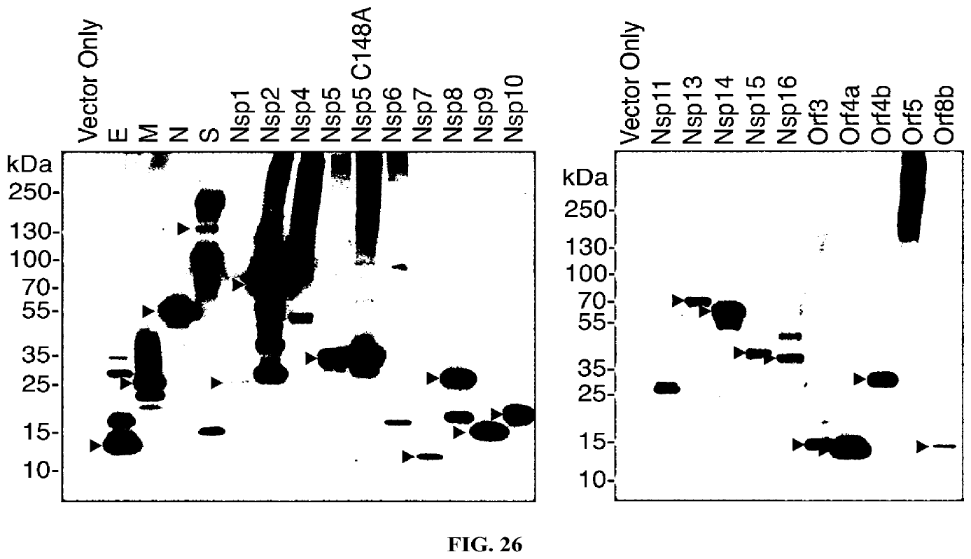

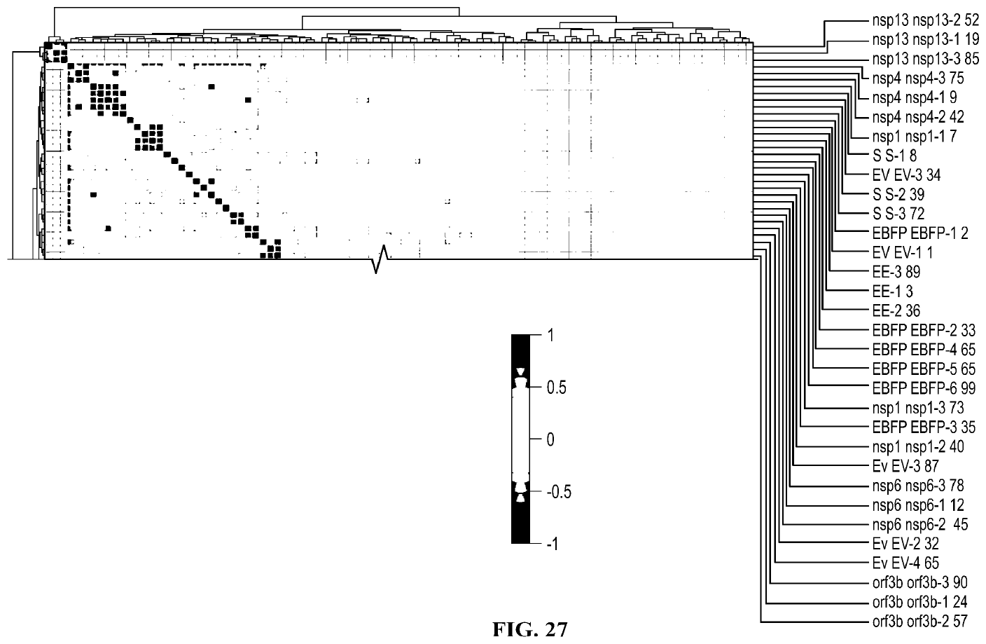

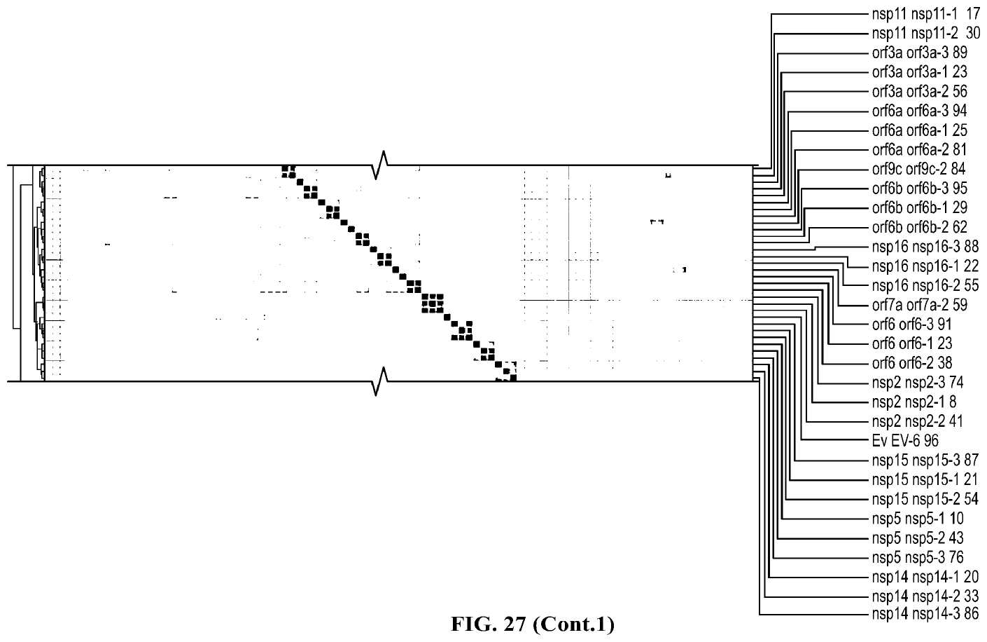

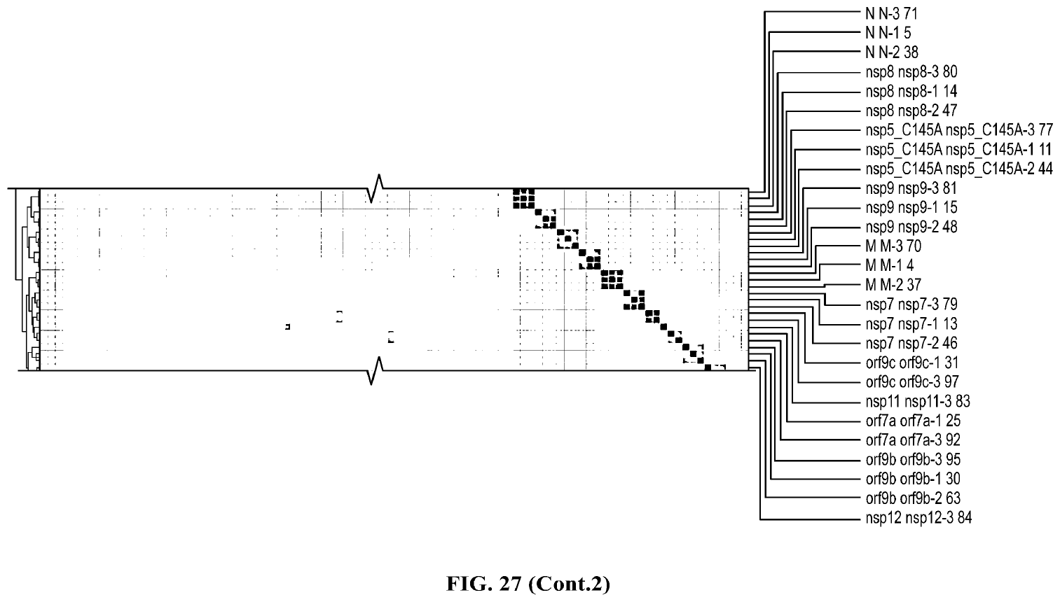

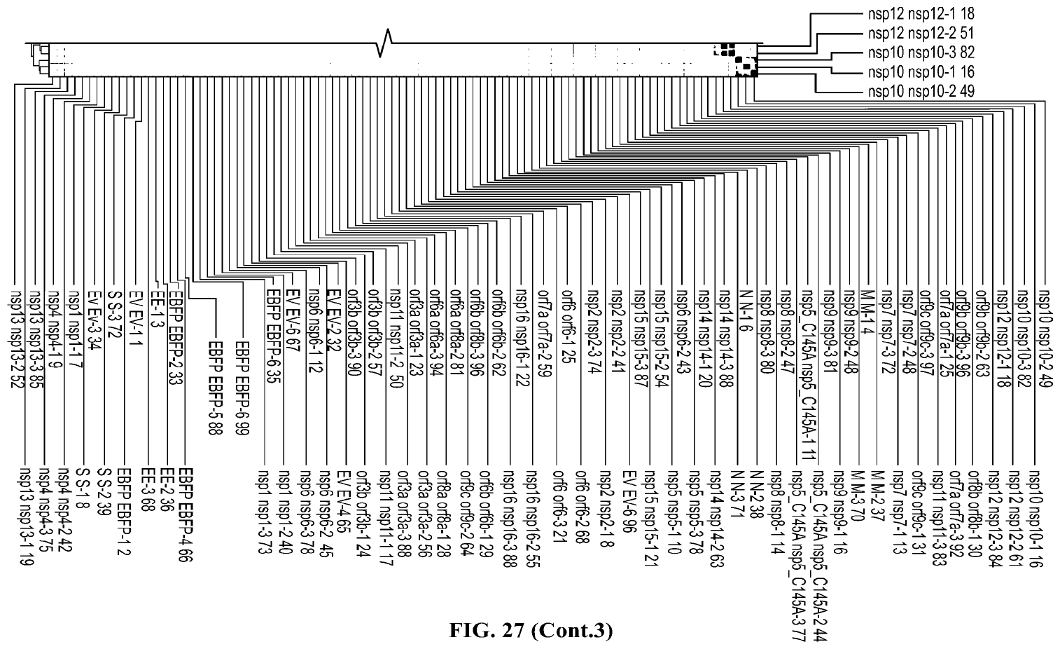

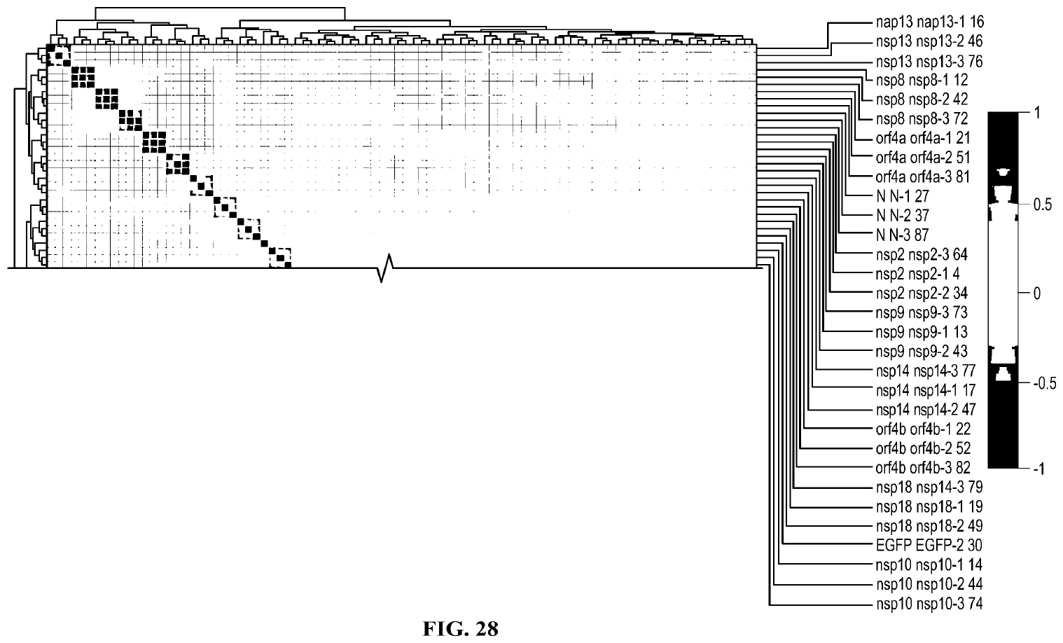

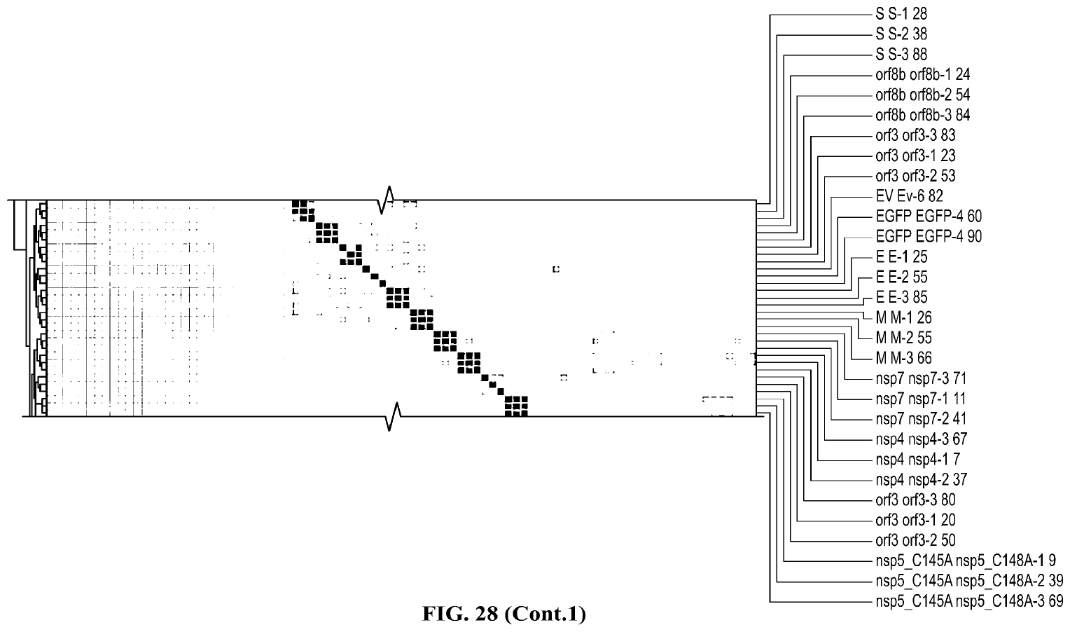

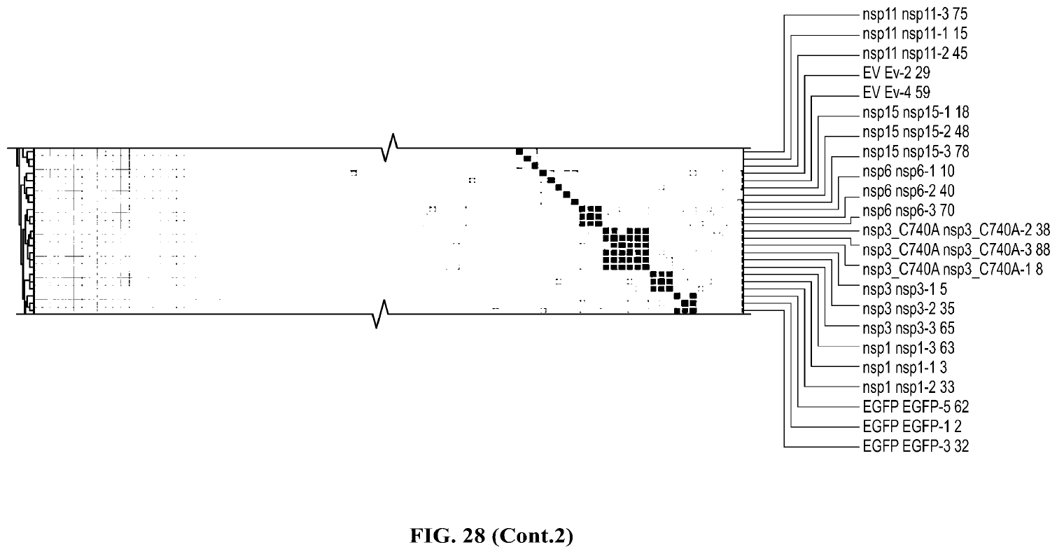

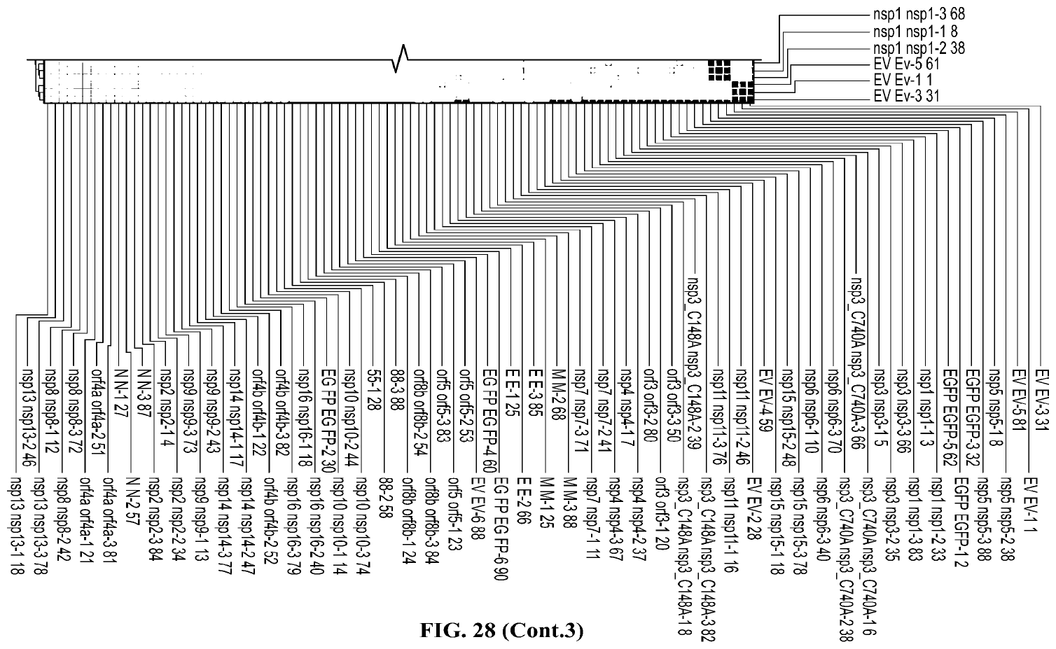

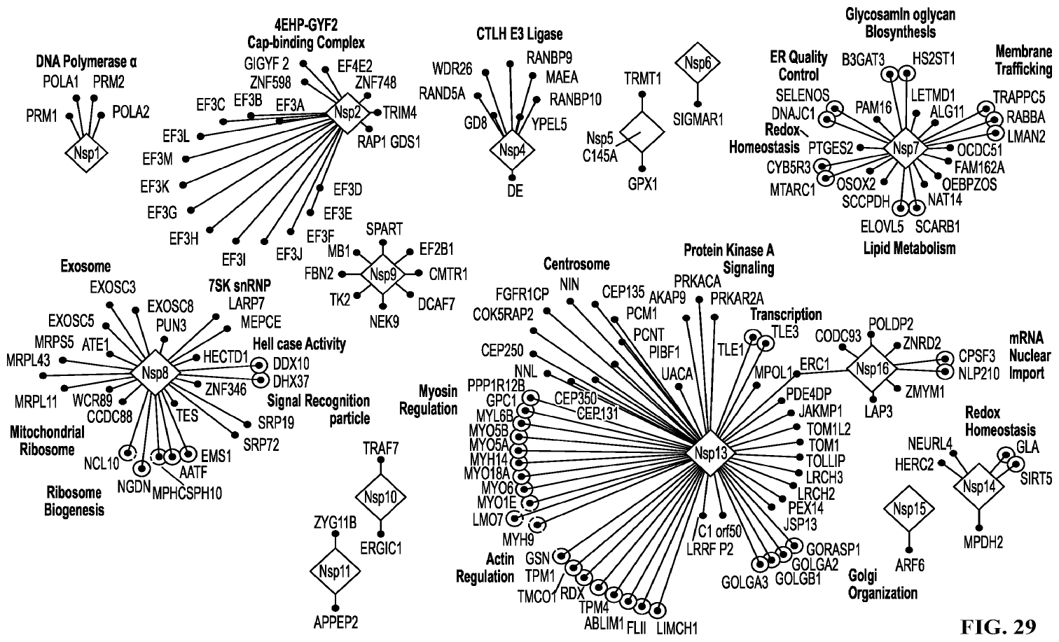

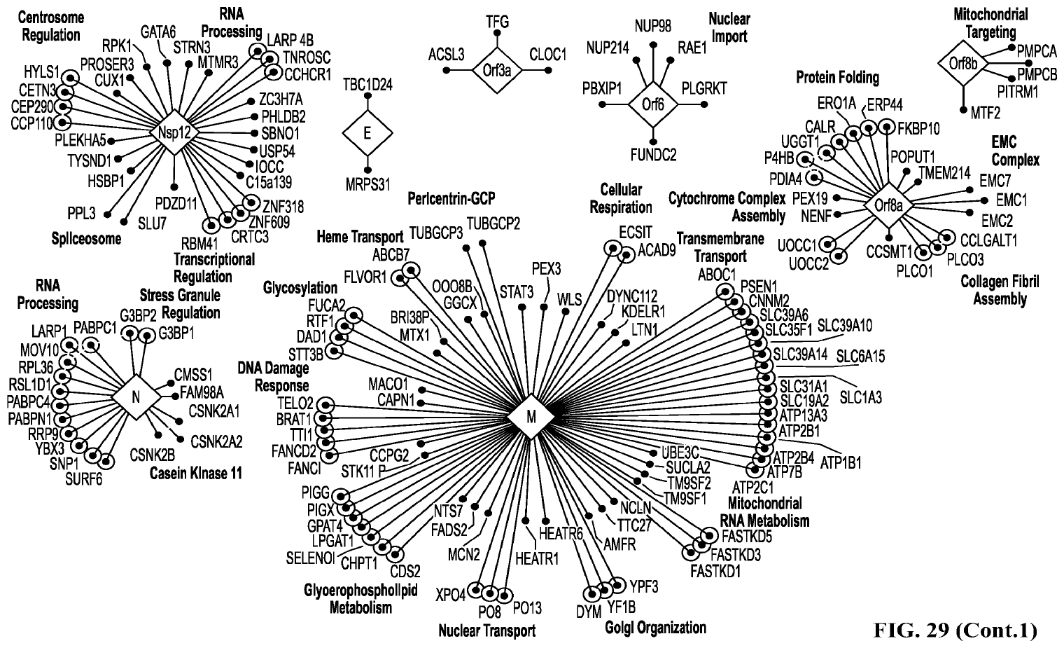

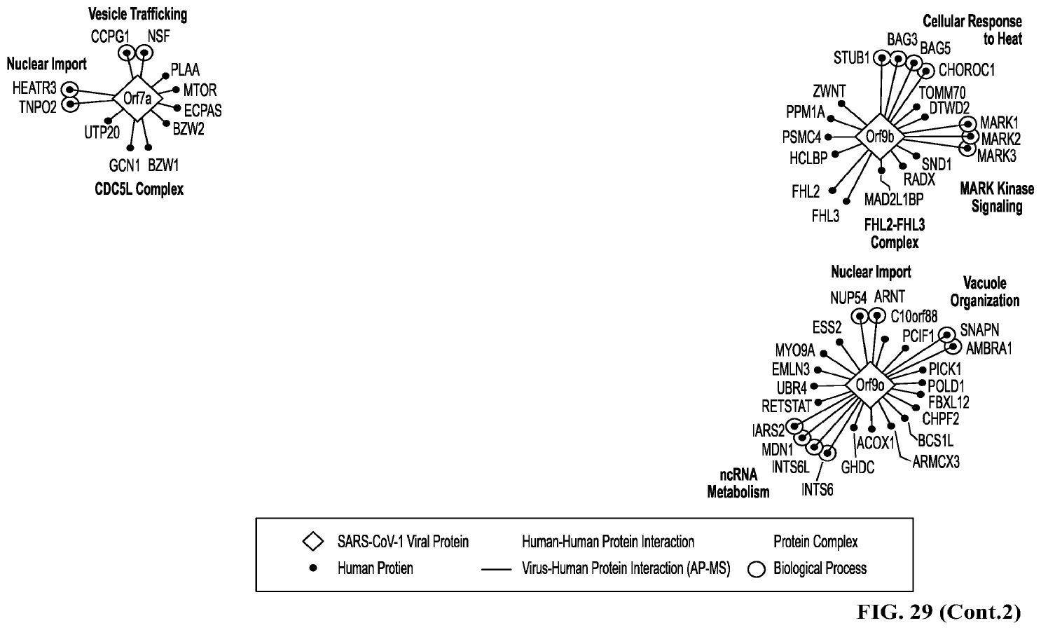

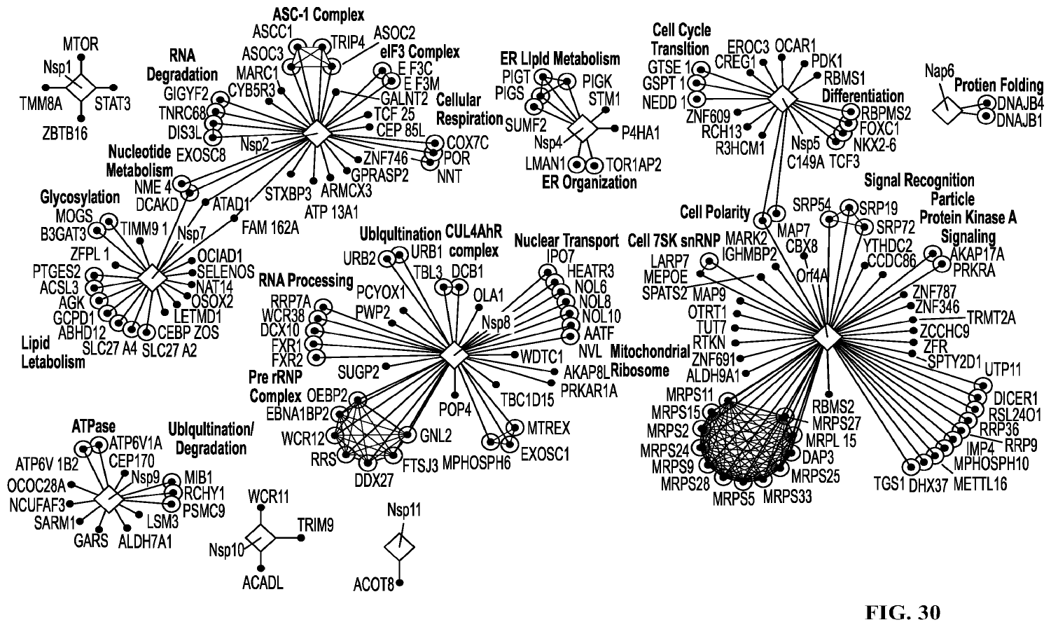

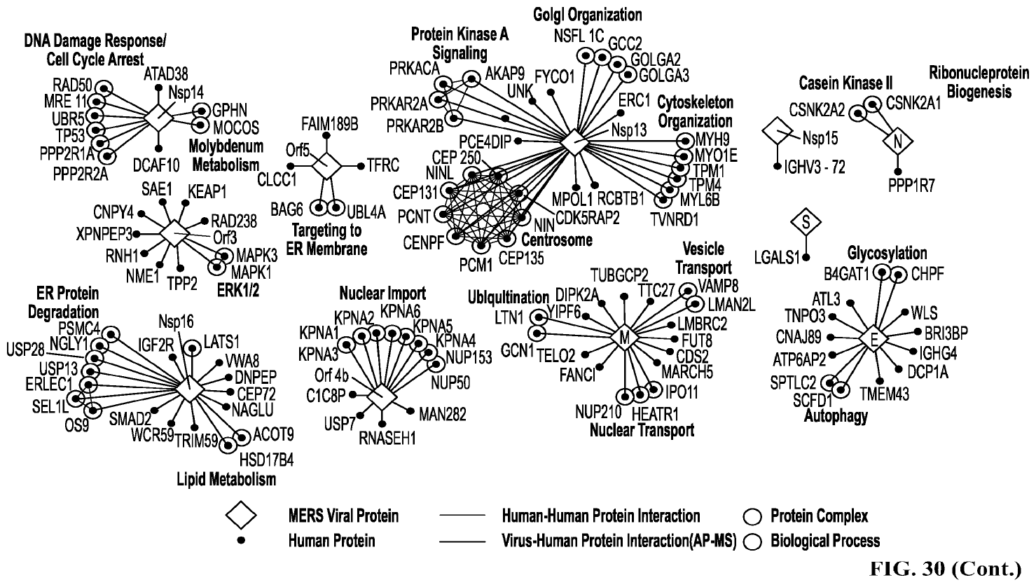

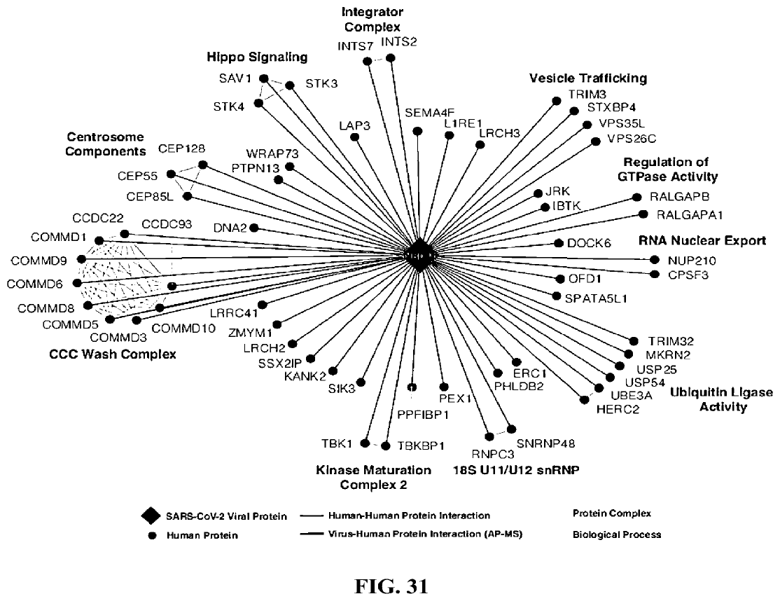

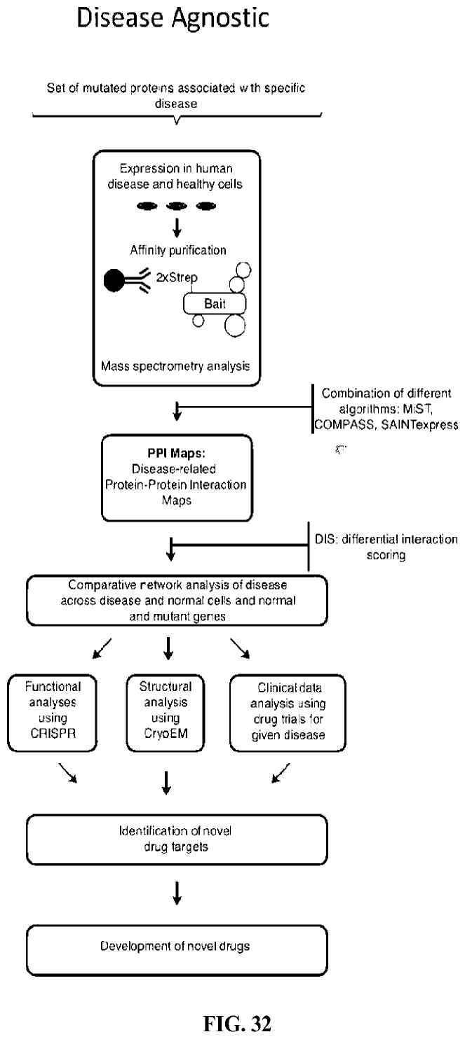

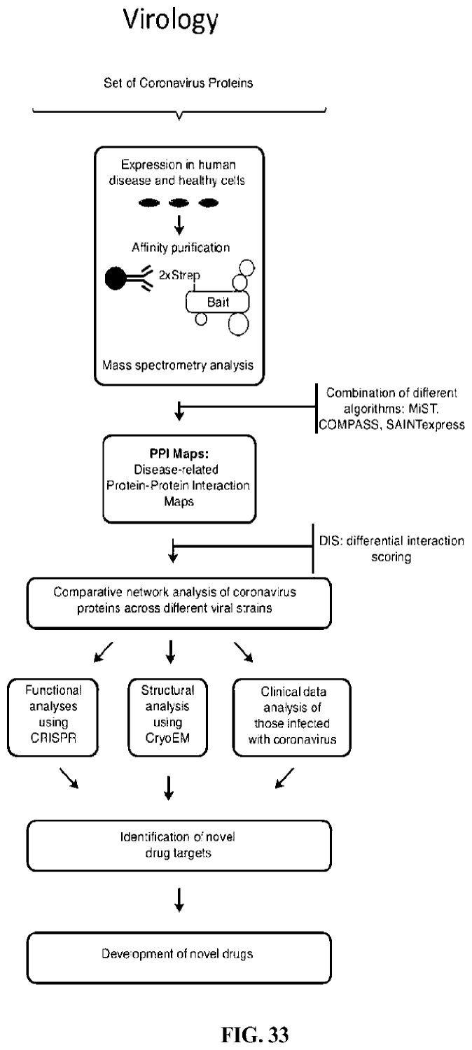

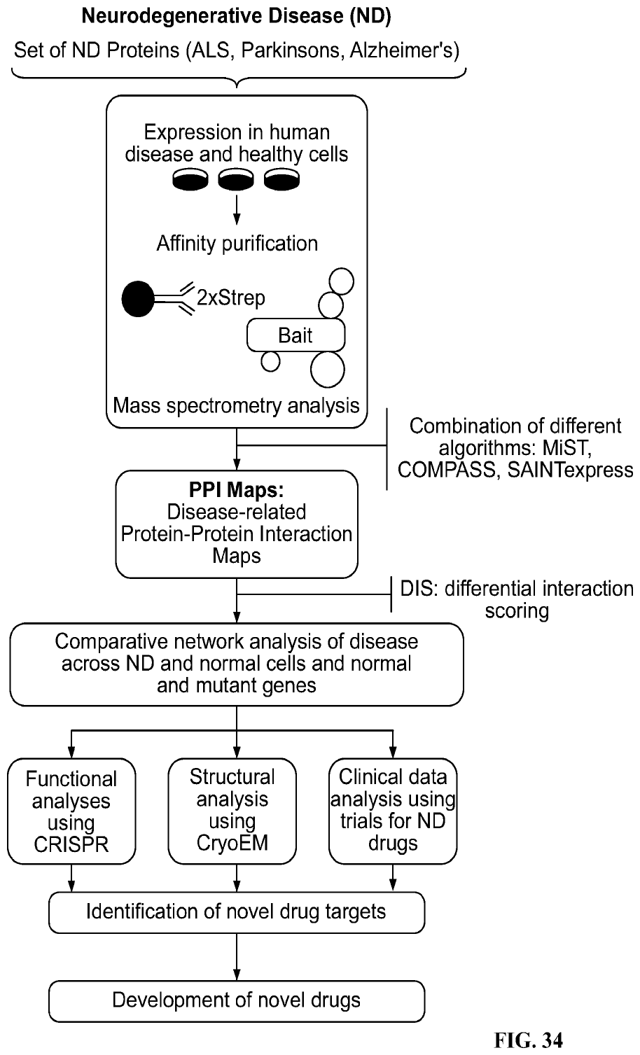

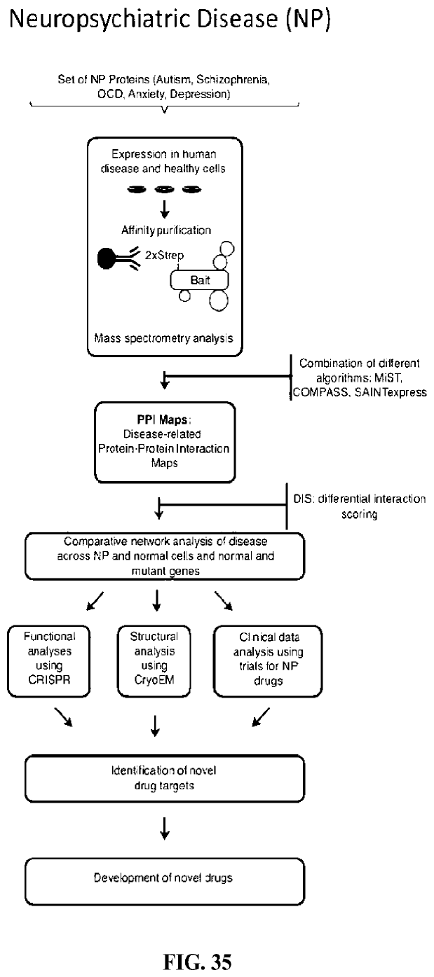

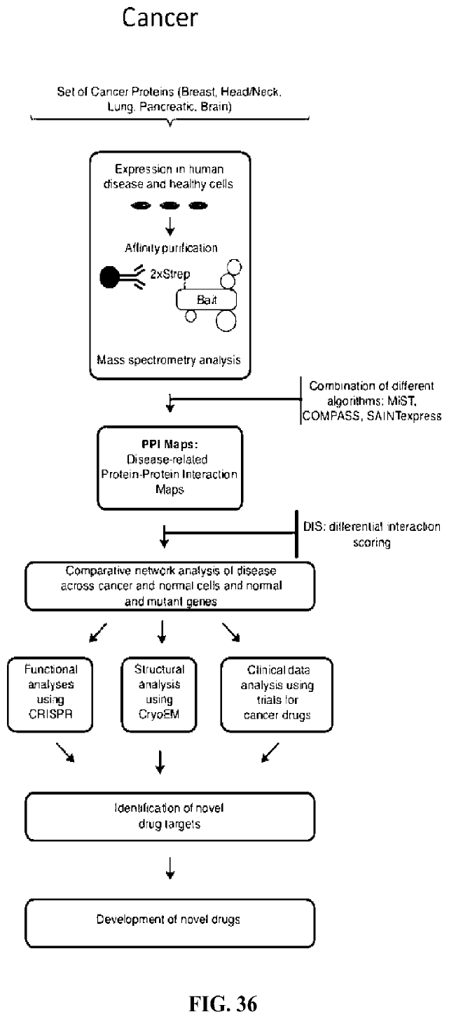

To create larger patient cohorts, drugs that shared activity against the same target, sigma receptors, were grouped. Sigma-1 and sigma-2 were previously identified as drug targets in the SARS-CoV-2-human protein-protein interaction map and multiple potent, non-selective sigma ligands were among the most promising inhibitors of SARS-CoV-2 replication in Vero E6 cells (D. E. Gordon, et al. Nature (2020)). As shown above, knockout and knockdown of SIGMAR1, but not SIGMAR2 (also known as TMEM97), led to robust decreases in SARS-CoV-2 replication (FIG. 4F and FIG. 22A-E), suggesting that sigma-1 may be a key therapeutic target. SIGMARJ sequences were analyzed across 359 mammals, and positive selection of several residues was observed within beaked whale, mouse, and ruminant lineages, which may indicate a role in host-pathogen competition (FIG. 24). Additionally, the sigma ligand drug amiodarone inhibited SARS-CoV-1 as well as SARS-CoV-2, consistent with the conservation of the Nsp6-sigma-1 interaction across the SARS viruses (FIG. 15D and FIG. 22A-E). Then, a search for other FDA-approved drugs with reported nanomolar affinity for sigma receptors or that fit the sigma ligand chemotype was conducted (D. E. Gordon, et al. Nature (2020); C. Abate, et al., A structure-affinity and comparative molecular field analysis of sigma-2 (sigma2) receptor ligands. Cent. Nerv. Syst. Agents Med. Chem. 9, 246-257 (2009); R. A. Glennon, Sigma receptor ligands and the use thereof. US Patent (2000), (available at https://patentimages.storage.googleapis.com/dc/36/68/73f4ccdac4c973/U.S. Pat. No. 6,057,371.pdf); R. R. Matsumoto, B. Pouw, Correlation between neuroleptic binding to sigma(1) and sigma(2) receptors and acute dystonic reactions. Eur. J. Pharmacol. 401, 155-160 (2000); M. Dold, et al., Haloperidol versus first-generation antipsychotics for the treatment of schizophrenia and other psychotic disorders. Cochrane Database Syst. Rev. 1, CD009831 (2015); F. F. Moebius, et al., Pharmacological analysis of sterol delta8-delta7 isomerase proteins with [3H]ifenprodil. Mol. Pharmacol. 54, 591-598 (1998); E. Gregori-Puigjané, et al.t, Identifying mechanism-of-action targets for drugs and probes. Proc. Natl. Acad. Sci. U S. A. 109, 11178-11183 (2012); Z. Hubler, et al., Accumulation of 8,9-unsaturated sterols drives oligodendrocyte formation and remyelination. Nature. 560, 372-376 (2018); F. F. Moebius, et al., High affinity of sigma 1-binding sites for sterol isomerization inhibitors: evidence for a pharmacological relationship with the yeast sterol C8-C7 isomerase. Br. J. Pharmacol. 121, 1-6 (1997)), and 12 such therapeutics were selected. It was found that all are potent inhibitors of SARS-CoV-2 with IC₅₀ values under 10 μM, though it is important to note that a wide range in sigma receptor affinity is seen, with no clear correlation between sigma receptor binding affinity and antiviral activity (FIG. 22D). Several clinical drug classes were represented by more than one candidate, including typical antipsychotics and antihistamines. Over-the-counter antihistamines are not well represented in medical billing data and are therefore poor candidates for real-world analysis, but users of typical antipsychotics can be easily identified in the patient cohort. By grouping these individual drug candidates by clinical indication, a better-powered comparison was built.

Referring to FIG. 24, Benjamini-Hochberg-corrected p-values (y-axis) for accelerated (blue circles) or conserved (green Xs) evolution at codons in SIGMAR1 in the denoted lineages relative to the neutral rate in mammals are shown.

A cohort for retrospective analysis on new, inpatient users of antipsychotics was constructed. In inpatient settings, typical and atypical antipsychotics are used similarly, most commonly for delirium. The effectiveness of typical antipsychotics, which have sigma activity and antiviral effects, versus atypical antipsychotics, which are not predicted to, was compared for treatment of COVID-19 (FIG. 23C). Observing mechanical ventilation outcomes in inpatient cohorts is a proxy for worsening of severe illness, rather than the progression from mild disease signified by the hospitalization of indomethacin-exposed outpatients above. RSS plus PS was again employed to build a robust, directly comparable cohort of inpatients (Table 7A-I). In the primary analysis, half as many new users of the sigma-ligand typical antipsychotics compared to new users of atypical antipsychotics progressed to the point of requiring mechanical ventilation, demonstrating significantly lower propensity with an odds ratio (OR) of 0.46 (95% CI=0.23-0.93, p=0.03, FIG. 23D). As above, a sensitivity analysis was conducted in the RSS-only cohort, and the same trend observed (OR=0.56, 95% CI=0.31-1.02, p=0.06), emphasizing the primary result of a beneficial effect for typical versus atypical antipsychotics observed in the RSS-plus-PS-matched cohort. Although a careful analysis of the relative benefits and risks of typical antipsychotics should be undertaken before considering prospective studies or interventions, these data and analysis demonstrate how molecular information can be translated into real-world implications for the treatment of COVID-19, an approach that can ultimately be applied to other diseases in the future.

Referring to FIG. 23C, a schematic of retrospective real-world clinical data analysis of typical antipsychotic use for inpatients with SARS-CoV-2 is shown. Plots show distribution of propensity scores for all included patients (red, typical users; blue, atypical users). For a full list of inclusion, exclusion, and matching criteria see Table 7A-I.

Referring to FIG. 23D, the effectiveness of typical vs. atypical antipsychotics among hospitalized patients with confirmed SARS-CoV-2 infection treated inhospital is shown. Average standardized absolute mean difference (ASAMD) is a measure of balance between typical and atypical groups calculated as the mean of the absolute standardized difference for each propensity score factor (Table 7A-I); p-value and odds ratios with 95% CI are estimated using the Aetion Evidence Platform r4.6. No ASAMD was greater than 0.1.

### Discussion

In this study, three different coronavirus-human protein-protein interaction maps were generated and compared in an attempt to identify and understand pan-coronavirus molecular mechanisms. The use of a quantitative differential interaction scoring (DIS) approach permitted the identification of virus-specific as well as shared interactions among distinct coronaviruses. Subcellular localization analysis was also systemically carried out using tagged viral proteins as well as antibodies targeting specific SARS-CoV-2 proteins.

These data were integrated with genetic data where the interactions uncovered with SARS-CoV-2 were perturbed using RNAi and CRISPR in different cellular systems and viral assays, an effort that functionally connected many host factors to infection. One of these, Tom70, which has been shown to bind to Orf9b from both SARS-CoV-1 and SARS-CoV-2, is a mitochondrial outer membrane translocase that has been previously shown to be important for mounting an interferon response (H.-W. Jiang, et al., SARS-CoV-2 Orf9b suppresses type I interferon responses by targeting TOM70. Cell. Mol. Immunol. 17, 998-1000 (2020)). These functional data, however, show that Tom70 has at least some role in promoting infection rather than inhibiting it. Using cryoEM, a 3 Å structure of a region of Orf9b binding to the active site of Tom70 was obtained. Remarkably, it was found that Orf9b is in a drastically different conformation than previously visualized. This offers the possibility that Orf9b may partition between two distinct structural states in the cells, with each possessing a different function and possibly explaining its potential functional pleiotropy. The exact details of functional significance and regulation of the Orf9b-Tom70 interaction await further experimental elucidation. This interaction, however, which is conserved between SARS-CoV-1 and SARS-CoV-2, could have value as a pan-coronavirus therapeutic target.

Finally, an attempt to connect the in vitro molecular data to clinical information available for COVID-19 patients was made to understand the pathophysiology of COVID-19 and explore new therapeutic avenues. To this end, using GWAS datasets of the COVID-19 Host Genetics Initiative (C. Huang, et al., The COVID-19 Host Genetics Initiative, a global initiative to elucidate the role of host genetic factors in susceptibility and severity of the SARS-CoV-2 virus pandemic. Eur. J. Hum. Genet. 28, 715-718 (2020)), it was observed that increased predicted sIL17RA plasma levels were associated with lower risk of COVID-19. Interestingly, it was found that IL17RA physically binds to SARS-CoV-2 Orf8 and genetic disruption results in decreased infection. Without wishing to be bound by theory, these collective data suggest that future studies should be focused on this pathway as both an indicator and therapeutic target for COVID-19. Furthermore, using medical billing data, trends in COVID-19 patients on specific drugs indicated by the molecular studies were also observed. For example, inpatients prescribed sigma-ligand typical antipsychotics seemingly have better COVID-19 outcomes when compared to users of atypical antipsychotics, which do not bind to sigma-1. It is uncertain whether sigma receptor interaction is the mechanism underpinning this effect, as typical antipsychotics are known to bind to a multitude of cellular targets. Replication in other patient cohorts and further work will be needed to see if there is therapeutic value in these connections, but at the very least a strategy has been demonstrated wherein protein network analyses can be used to make testable predictions from real-world, clinical information.

Overall, an integrative and collaborative approach to study and understand pathogenic coronavirus infection is described, identifying conserved targeted mechanisms that are likely to be of high relevance for other viruses of this family. Proteomics, cell biology, virology, genetics, structural biology, biochemistry, and clinical and genomic information was used in an attempt to provide a holistic view of SARS-CoV-2 and other coronaviruses' interactions with infected host cells. Without wishing to be bound by theory, it is proposed that such an integrative and collaborative approach could and should be used to study other infectious agents as well as other disease areas.

### Additional Exemplifications

In some embodiments, it is envisioned that the methods and systems disclosed herein can be used on a variety of different diseases, uncovering new biology and ultimately novel targets as well as new drugs. For example, the integrated suite of technologies disclosed herein will be focused on neurodegenerative diseases (e.g., Parkinsons disease, Amyotrophic Lateral Sclerosis, Alzheimer's disease) and neuropsychiatric disorders (e.g., autism, schizophrenia, obsessive compulsive disorder, depression). A number of cancers will also be studied, including lung, brain, and pancreatic cancers. Finally, additional efforts will be placed on pathogens, both bacterial and viral, with a focus on coronaviruses and other viruses that could result in future pandemics.

Exemplary genes and cell lines that can be utilized in focusing on neurodegenerative diseases are listed in Table 13A and Table 13B, respectively.

TABLE 13A

| | | GenBank ID or |
|---|---|---|
| Indication | Gene | Ensembl ID |
| Amyotrophic Lateral Sclerosis | SOD1 | 6647 |
| (ALS) | | |
| Amyotrophic Lateral Sclerosis | ALS2 | 57679 |
| (ALS) | | |
| Amyotrophic Lateral Sclerosis | SETX | 23064 |
| (ALS) | | |
| Amyotrophic Lateral Sclerosis | SPG11 | 80208 |
| (ALS) | | |
| Amyotrophic Lateral Sclerosis | FUS | 2521 |
| (ALS) | | |
| Amyotrophic Lateral Sclerosis | VAPB | 9217 |
| (ALS) | | |
| Amyotrophic Lateral Sclerosis | ANG | 283 |
| (ALS) | | |
| Amyotrophic Lateral Sclerosis | TARDBP | 23435 |
| (ALS) | | |
| Amyotrophic Lateral Sclerosis | FIG4 | 9896 |
| (ALS) | | |
| Amyotrophic Lateral Sclerosis | OPTN | 10133 |
| (ALS) | | |
| Amyotrophic Lateral Sclerosis | ATXN2 | 6311 |
| (ALS) | | |
| Amyotrophic Lateral Sclerosis | VCP | 7415 |
| (ALS) | | |
| Amyotrophic Lateral Sclerosis | UBQLN2 | 29978 |
| (ALS) | | |
| Amyotrophic Lateral Sclerosis | SIGMAR1 | 10280 |
| (ALS) | | |
| Amyotrophic Lateral Sclerosis | CHMP2B | 25978 |
| (ALS) | | |
| Amyotrophic Lateral Sclerosis | PFN1 | 5216 |
| (ALS) | | |
| Amyotrophic Lateral Sclerosis | ERBB4 | 2066 |
| (ALS) | | |
| Amyotrophic Lateral Sclerosis | HNRNPA1 | 3178 |
| (ALS) | | |
| Amyotrophic Lateral Sclerosis | MATR3 | 9782 |
| (ALS) | | |
| Amyotrophic Lateral Sclerosis | TUBA4A | 7277 |
| (ALS) | | |
| Amyotrophic Lateral Sclerosis | ANXA11 | 311 |
| (ALS) | | |
| Amyotrophic Lateral Sclerosis | NEK1 | 4750 |
| (ALS) | | |
| Amyotrophic Lateral Sclerosis | C9orf72 | 203228 |
| (ALS) | | |
| Amyotrophic Lateral Sclerosis | CHCHD10 | 400916 |
| (ALS) | | |
| Amyotrophic Lateral Sclerosis | SQSTM1 | 8878 |
| (ALS) | | |
| Alzheimer's disease (AD) | APOE | 348 |
| Alzheimer's disease (AD) | CD2AP | 23607 |
| Alzheimer's disease (AD) | ABCA7 | 10347 |
| Alzheimer's disease (AD) | CLU | 1191 |
| Alzheimer's disease (AD) | CR1 | 1378 |
| Alzheimer's disease (AD) | PICALM | 8301 |
| Alzheimer's disease (AD) | PLD3 | 23646 |
| Alzheimer's disease (AD) | TREM2 | 54209 |
| Alzheimer's disease (AD) | SORL1 | 6653 |
| Alzheimer's disease (AD) | APP | 351 |
| Alzheimer's disease (AD) | PSEN1 | 5663 |
| Alzheimer's disease (AD) | PSEN2 | 5664 |
| Alzheimer's disease (AD) | RUFY1 | 80230 |
| Alzheimer's disease (AD) | PSD2 | 84249 |
| Alzheimer's disease (AD) | TCIRG1 | 10312 |
| Alzheimer's disease (AD) | RIN3 | 79890 |
| Alzheimer's disease (AD) | STH | 246744 |
| Alzheimer's disease (AD) | CLU | 1191 |
| Alzheimer's disease (AD) | PICALM | 8301 |
| Alzheimer's disease (AD) | BIN1 | 274 |
| Alzheimer's disease (AD) | EPHA1 | 2041 |
| Alzheimer's disease (AD) | SORL1 | 6653 |
| Alzheimer's disease (AD) | ABI3 | 51225 |
| Parkinson's Disease (PD) | LRRK2 | 120892 |
| Parkinson's Disease (PD) | PINK1 | 65018 |
| Parkinson's Disease (PD) | PRKN | 5071 |
| Parkinson's Disease (PD) | SNCA | 6622 |
| Parkinson's Disease (PD) | GBA | 2629 |
| Parkinson's Disease (PD) | UCHL1 | 7345 |
| Parkinson's Disease (PD) | ATP13A2 | 23400 |
| Parkinson's Disease (PD) | VPS35 | 55737 |
| Parkinson's Disease (PD) | PARK3 | 5072 |
| Parkinson's Disease (PD) | DJ-1 | 11315 |
| Parkinson's Disease (PD) | PARK10 | 170534 |
| Parkinson's Disease (PD) | PARK11 | 26058 |
| Parkinson's Disease (PD) | PARK12 | 677662 |
| Parkinson's Disease (PD) | HTRA2 | 27429 |
| Parkinson's Disease (PD) | PLA2G6 | 8398 |
| Parkinson's Disease (PD) | FBX07 | 25793 |
| Parkinson's Disease (PD) | PARK16 | 100359403 |
| Parkinson's Disease (PD) | EIF4G1 | 1981 |

TABLE 13B

| Indication | Cell Lines |
|---|---|
| Amyotrophic Lateral | WC034i-SOD1-D90A |
| Sclerosis (ALS) | |
| Amyotrophic Lateral | WC035i-SOD1-D90D |
| Sclerosis (ALS) | |
| Amyotrophic Lateral | Human iPSC-derived neural |
| Sclerosis (ALS) | stem cells |
| Amyotrophic Lateral | HEK293T |
| Sclerosis (ALS) | |
| Alzheimer's disease | Human iPSC-derived neural |
| (AD) | stem cells |
| Alzheimer's disease | HEK293T |
| (AD) | |
| Parkinson's Disease | Human iPSC-derived neural |
| (PD) | stem cells |
| Parkinson's Disease | HEK293T |
| (PD) | |

Exemplary genes and cell lines that can be utiliz5d in focusing on neuropsychiatric disorders are listed in Table 14A and Table 14B, respectively.

TABLE 14A

| | | GenBank ID or |
|---|---|---|
| Indication | Gene | Ensembl ID |
| Autism | CHD8 | 57680 |
| Autism | SCN2A | 6326 |
| Autism | SYNGAP1 | 8831 |
| Autism | ADNP | 23394 |
| Autism | FOXP1 | 27086 |
| Autism | POGZ | 23126 |
| Autism | ARID1B | 57492 |
| Autism | SUV420H1 | 51111 |
| Autism | DYRK1A | 1859 |
| Autism | SLC6A1 | 6529 |
| Autism | GRIN2B | 2904 |
| Autism | PTEN | 5728 |
| Autism | SHANK3 | 85358 |
| Autism | MED13L | 23389 |
| Autism | GIGYF1 | 64599 |
| Autism | CHD2 | 1106 |
| Autism | ANKRD11 | 29123 |
| Autism | ANK2 | 287 |
| Autism | ASH1L | 55870 |
| Autism | TLK2 | 11011 |
| Autism | DNMT3A | 1788 |
| Autism | DEAF1 | 10522 |
| Autism | CTNNB1 | 1499 |
| Autism | KDM6B | 23135 |
| Autism | DSCAM | 1826 |
| Autism | SETD5 | 55209 |
| Autism | KCNQ3 | 3786 |
| Autism | SRPR | 6734 |
| Autism | KDM5B | 10765 |
| Autism | WAC | 51322 |
| Autism | SHANK2 | 22941 |
| Autism | NRXN1 | 9378 |
| Autism | TBL1XR1 | 79718 |
| Autism | MYTIL | 23040 |
| Autism | BCL11A | 53335 |
| Autism | RORB | 6096 |
| Autism | RAI1 | 10743 |
| Autism | DYNC1H1 | 1778 |
| Autism | DPYSL2 | 1808 |
| Autism | AP2S1 | 1175 |
| Autism | KMT2C | 58508 |
| Autism | PAX5 | 5079 |
| Autism | MKX | 283078 |
| Autism | GABRB3 | 2562 |
| Autism | SIN3A | 25942 |
| Autism | MBD5 | 55777 |
| Autism | MAP1A | 4130 |
| Autism | STXBP1 | 6812 |
| Autism | CELF4 | 56853 |
| Autism | PHF12 | 57649 |
| Autism | TBR1 | 10716 |
| Autism | PPP2R5D | 5528 |
| Autism | TM9SF4 | 9777 |
| Autism | PHF21A | 51317 |
| Autism | PRR12 | 57479 |
| Autism | SKI | 6497 |
| Autism | ASXL3 | 80816 |
| Autism | SPAST | 6683 |
| Autism | SMARCC2 | 6601 |
| Autism | TRIP12 | 9320 |
| Autism | CREBBP | 1387 |
| Autism | TCF4 | 6925 |
| Autism | CACNA1E | 777 |
| Autism | GNAI1 | 2770 |
| Autism | TCF20 | 6942 |
| Autism | FOXP2 | 93986 |
| Autism | NSD1 | 64324 |
| Autism | TCF7L2 | 6934 |
| Autism | LDB1 | 8861 |
| Autism | EIF3G | 8666 |
| Autism | PHF2 | 5253 |
| Autism | KIAA0232 | 9778 |
| Autism | VEZF1 | 7716 |
| Autism | GFAP | 2670 |
| Autism | IRF2BPL | 64207 |
| Autism | ZMYND8 | 23613 |
| Autism | SATB1 | 6304 |
| Autism | RFX3 | 5991 |
| Autism | SCN1A | 6323 |
| Autism | PPP5C | 5536 |
| Autism | TRIM23 | 373 |
| Autism | TRAF7 | 84231 |
| Autism | ELAVL3 | 1995 |
| Autism | GRIA2 | 2891 |
| Autism | LRRC4C | 57689 |
| Autism | CACNA2D3 | 55799 |
| Autism | NUP155 | 9631 |
| Autism | KMT2E | 55904 |
| Autism | NR3C2 | 4306 |
| Autism | NACC1 | 112939 |
| Autism | PTK7 | 5754 |
| Autism | PPP1R9B | 84687 |
| Autism | GABRB2 | 2561 |
| Autism | HDLBP | 3069 |
| Autism | TAOK1 | 57551 |
| Autism | UBR1 | 197131 |
| Autism | TEK | 7010 |
| Autism | KCNMA1 | 3778 |
| Autism | CORO1A | 11151 |
| Autism | HECTD4 | 283450 |
| Autism | NCOA1 | 8648 |
| Autism | DIP2A | 23181 |

TABLE 14B

| Indication | Cell Lines |
|---|---|
| Autism | HEK293T |
| Autism | NPCs |

Exemplary genes and cell lines that can be utilized in focusing on cancer are listed in Table 15A and Table 15B, respectively.

TABLE 15A

| | | GenBank ID or |
|---|---|---|
| Indication | Gene | Ensembl ID |
| Glioblastoma | PTEN | ENSG00000171862 |
| Glioblastoma | TTN | ENSG00000155657 |
| Glioblastoma | TP53 | ENSG00000141510 |
| Glioblastoma | EGFR | ENSG00000146648 |
| Glioblastoma | FLG | ENSG00000143631 |
| Glioblastoma | MUC16 | ENSG00000181143 |
| Glioblastoma | NF1 | ENSG00000196712 |
| Glioblastoma | RYR2 | ENSG00000198626 |
| Glioblastoma | PKHD1 | ENSG00000170927 |
| Glioblastoma | HMCN1 | ENSG00000143341 |
| Glioblastoma | SYNE1 | ENSG00000131018 |
| Glioblastoma | SPTA1 | ENSG00000163554 |
| Glioblastoma | PIK3R1 | ENSG00000145675 |
| Glioblastoma | RB1 | ENSG00000139687 |
| Glioblastoma | ATRX | ENSG00000085224 |
| Glioblastoma | PIK3CA | ENSG00000121879 |
| Glioblastoma | OBSCN | ENSG00000154358 |
| Glioblastoma | APOB | ENSG00000084674 |
| Glioblastoma | FLG2 | ENSG00000143520 |
| Glioblastoma | LRP2 | ENSG00000081479 |
| Glioblastoma | USH2A | ENSG00000042781 |
| Glioblastoma | LAMA1 | ENSG00000101680 |
| Glioblastoma | PCLO | ENSG00000186472 |
| Glioblastoma | DNAHS | ENSG00000039139 |
| Glioblastoma | MUC17 | ENSG00000169876 |
| Glioblastoma | DNAH3 | ENSG00000158486 |
| Glioblastoma | COL6A3 | ENSG00000163359 |
| Glioblastoma | DNAH2 | ENSG00000183914 |
| Glioblastoma | TRRAP | ENSG00000196367 |
| Glioblastoma | DST | ENSG00000151914 |
| Glioblastoma | HRNR | ENSG00000197915 |
| Glioblastoma | KMT2C | ENSG00000055609 |
| Glioblastoma | FCGBP | ENSG00000275395 |
| Glioblastoma | SDK1 | ENSG00000146555 |
| Glioblastoma | GRIN2A | ENSG00000183454 |
| Glioblastoma | SYNE2 | ENSG00000054654 |
| Glioblastoma | AHNAK | ENSG00000124942 |
| Glioblastoma | RELN | ENSG00000189056 |
| Glioblastoma | MXRA5 | ENSG00000101825 |
| Glioblastoma | DNAH8 | ENSG00000124721 |
| Glioblastoma | DNAH9 | ENSG00000007174 |
| Glioblastoma | RYR3 | ENSG00000198838 |
| Glioblastoma | TAF1L | ENSG00000122728 |
| Glioblastoma | FAT2 | ENSG00000086570 |
| Glioblastoma | HYDIN | ENSG00000157423 |
| Glioblastoma | AHNAK2 | ENSG00000185567 |
| Glioblastoma | EP400 | ENSG00000183495 |
| Glioblastoma | TMEM132D | ENSG00000151952 |
| Glioblastoma | IDH1 | ENSG00000138413 |
| Glioblastoma | DNAH11 | ENSG00000105877 |
| Glioblastoma | PDZD2 | ENSG00000133401 |
| Glioblastoma | PDGFRA | ENSG00000134853 |
| Glioblastoma | DOCK5 | ENSG00000147459 |
| Glioblastoma | PIK3CG | ENSG00000105851 |
| Glioblastoma | ADAM29 | ENSG00000168594 |
| Glioblastoma | FRAS1 | ENSG00000138759 |
| Glioblastoma | ESPL1 | ENSG00000135476 |
| Glioblastoma | SACS | ENSG00000151835 |
| Glioblastoma | FAT4 | ENSG00000196159 |
| Glioblastoma | CFAP4Z | ENSG00000165164 |
| Glioblastoma | ANK2 | ENSG00000145362 |
| Glioblastoma | CSMD2 | ENSG00000121904 |
| Glioblastoma | RIMS2 | ENSG00000176406 |
| Glioblastoma | ZNF318 | ENSG00000171467 |
| Glioblastoma | NOS1 | ENSG00000089250 |
| Glioblastoma | LRP1 | ENSG00000123384 |
| Glioblastoma | HCN1 | ENSG00000164588 |
| Glioblastoma | PKDREJ | ENSG00000130943 |
| Glioblastoma | VWF | ENSG00000110799 |
| Glioblastoma | DSP | ENSG00000096696 |
| Glioblastoma | CNTNAP2 | ENSG00000174469 |
| Glioblastoma | HSPG2 | ENSG00000142798 |
| Glioblastoma | TSHZ2 | ENSG00000182463 |
| Glioblastoma | ZFHX3 | ENSG00000140836 |
| Glioblastoma | LCT | ENSG00000115850 |
| Glioblastoma | SPHKAP | ENSG00000153820 |
| Glioblastoma | ADAMTS12 | ENSG00000151388 |
| Glioblastoma | UBR4 | ENSG00000127481 |
| Glioblastoma | KIF2B | ENSG00000141200 |
| Glioblastoma | RYR1 | ENSG00000196218 |
| Glioblastoma | GRM3 | ENSG00000198822 |
| Glioblastoma | LRRK1 | ENSG00000154237 |
| Glioblastoma | ADGRV1 | ENSG00000164199 |
| Glioblastoma | SLIT3 | ENSG00000184347 |
| Glioblastoma | KMT2A | ENSG00000118058 |
| Glioblastoma | PLCG2 | ENSG00000197943 |
| Glioblastoma | ANK3 | ENSG00000151150 |
| Glioblastoma | WBSCR17 | ENSG00000185274 |
| Glioblastoma | TCHH | ENSG00000159450 |
| Glioblastoma | MYH2 | ENSG00000125414 |
| Glioblastoma | MYH11 | ENSG00000133392 |
| Glioblastoma | NLRP7 | ENSG00000167634 |
| Glioblastoma | TSHZ3 | ENSG00000121297 |
| Glioblastoma | PRDM9 | ENSG00000164256 |
| Glioblastoma | UNC79 | ENSG00000133958 |
| Glioblastoma | COL1A2 | ENSG00000164692 |
| Glioblastoma | HERC2P3 | ENSG00000180229 |
| Glioblastoma | KANK1 | ENSG00000107104 |
| Glioblastoma | RNF213 | ENSG00000173821 |
| Glioblastoma | ATP10B | ENSG00000118322 |
| Pancreatic | KRAS | ENSG00000133703 |
| Pancreatic | TP53 | ENSG00000141510 |
| Pancreatic | SMAD4 | ENSG00000141646 |
| Pancreatic | CDKN2A | ENSG00000147889 |
| Pancreatic | TTN | ENSG00000155657 |
| Pancreatic | DNM1P47 | ENSG00000259660 |
| Pancreatic | MUC16 | ENSG00000181143 |
| Pancreatic | RNF43 | ENSG00000108375 |
| Pancreatic | CSMD2 | ENSG00000121904 |
| Pancreatic | RNF213 | ENSG00000173821 |
| Pancreatic | RYR1 | ENSG00000196218 |
| Pancreatic | GLI3 | ENSG00000106571 |
| Pancreatic | DNAH11 | ENSG00000105877 |
| Pancreatic | SCNSA | ENSG00000183873 |
| Pancreatic | OBSCN | ENSG00000154358 |
| Pancreatic | GNAS | ENSG00000087460 |
| Pancreatic | ARID1A | ENSG00000117713 |
| Pancreatic | RREB1 | ENSG00000124782 |
| Pancreatic | FLG | ENSG00000143631 |
| Pancreatic | CACNA1B | ENSG00000148408 |
| Pancreatic | USH2A | ENSG00000042781 |
| Pancreatic | CSMD3 | ENSG00000164796 |
| Pancreatic | PCDH15 | ENSG00000150275 |
| Pancreatic | LRP1B | ENSG00000168702 |
| Pancreatic | COL6A2 | ENSG00000142173 |
| Pancreatic | APOB | ENSG00000084674 |
| Pancreatic | FBN3 | ENSG00000142449 |
| Pancreatic | SYNE1 | ENSG00000131018 |
| Pancreatic | MACE1 | ENSG00000127603 |
| Pancreatic | COL5A1 | ENSG00000130635 |
| Pancreatic | SDK1 | ENSG00000146555 |
| Pancreatic | ADAMTS16 | ENSG00000145536 |
| Pancreatic | ATP10A | ENSG00000206190 |
| Pancreatic | ZFHX4 | ENSG00000091656 |
| Pancreatic | TGFBR2 | ENSG00000163513 |
| Pancreatic | ADAMTS12 | ENSG00000151388 |
| Pancreatic | KCNA6 | ENSG00000151079 |
| Pancreatic | KMT2D | ENSG00000167548 |
| Pancreatic | FAT2 | ENSG00000086570 |
| Pancreatic | MYO18B | ENSG00000133454 |
| Pancreatic | HMCN1 | ENSG00000143341 |
| Pancreatic | HECW2 | ENSG00000138411 |
| Pancreatic | FAT3 | ENSG00000165323 |
| Pancreatic | ATM | ENSG00000149311 |
| Pancreatic | PCDHB7 | ENSG00000113212 |
| Pancreatic | KIF1A | ENSG00000130294 |
| Pancreatic | PEG3 | ENSG00000198300 |
| Pancreatic | PLEC | ENSG00000178209 |
| Pancreatic | DCHS1 | ENSG00000166341 |
| Pancreatic | TPO | ENSG00000115705 |
| Pancreatic | ADGRD1 | ENSG00000111452 |
| Pancreatic | DSI | ENSG00000151914 |
| Pancreatic | FLNC | ENSG00000128591 |
| Pancreatic | PCDHA9 | ENSG00000204961 |
| Pancreatic | RIMS2 | ENSG00000176406 |
| Pancreatic | NOS1 | ENSG00000089250 |
| Pancreatic | KCNB2 | ENSG00000182674 |
| Pancreatic | LRP1 | ENSG00000123384 |
| Pancreatic | SSPO | ENSG00000197558 |
| Pancreatic | RP1 | ENSG00000104237 |
| Pancreatic | DSCAM | ENSG00000171587 |
| Pancreatic | MTUS2 | ENSG00000132938 |
| Pancreatic | RYR3 | ENSG00000198838 |
| Pancreatic | CSMD1 | ENSG00000183117 |
| Pancreatic | FN1 | ENSG00000115414 |
| Pancreatic | NYNG1 | ENSG00000162631 |
| Pancreatic | RELN | ENSG00000189056 |
| Pancreatic | MYLK | ENSG00000065534 |
| Pancreatic | MYO16 | ENSG00000041515 |
| Pancreatic | KDM6A | ENSG00000147050 |
| Pancreatic | FLT4 | ENSG00000037280 |
| Pancreatic | ATR | ENSG00000175054 |
| Pancreatic | CMYA5 | ENSG00000164309 |
| Pancreatic | TMEM132D | ENSG00000151952 |
| Pancreatic | APBA2 | ENSG00000034053 |
| Pancreatic | ABCA4 | ENSG00000198691 |
| Pancreatic | MUC17 | ENSG00000169876 |
| Pancreatic | PCDH9 | ENSG00000184226 |
| Pancreatic | WDR17 | ENSG00000150627 |
| Pancreatic | PKD1 | ENSG00000008710 |
| Pancreatic | COL22A1 | ENSG00000169436 |
| Pancreatic | PBRM1 | ENSG00000163939 |
| Pancreatic | SCN9A | ENSG00000169432 |
| Pancreatic | SORCS2 | ENSG00000184985 |
| Pancreatic | PTCHD2 | ENSG00000204624 |
| Pancreatic | MEFV | ENSG00000103313 |
| Pancreatic | KCNT1 | ENSG00000107147 |
| Pancreatic | PSG7 | ENSG00000221878 |
| Pancreatic | NLRP2 | ENSG00000022556 |
| Pancreatic | POM121L12 | ENSG00000221900 |
| Pancreatic | CUBN | ENSG00000107611 |
| Pancreatic | ANK3 | ENSG00000151150 |
| Pancreatic | NRXN3 | ENSG00000021645 |
| Pancreatic | ADGRL2 | ENSG00000117114 |
| Pancreatic | TENM3 | ENSG00000218336 |
| Pancreatic | ADAMTSL4 | ENSG00000143382 |
| Pancreatic | AKAP6 | ENSG00000151320 |
| Pancreatic | DPP6 | ENSG00000130226 |
| Pancreatic | TRPS1 | ENSG00000104447 |
| Pancreatic | SACS | ENSG00000151835 |
| Lung | TP53 | ENSG00000141510 |
| Lung | TTN | ENSG00000155657 |
| Lung | MUC16 | ENSG00000181143 |
| Lung | CSMD3 | ENSG00000164796 |
| Lung | RYR2 | ENSG00000198626 |
| Lung | SYNE1 | ENSG00000131018 |
| Lung | LRP1B | ENSG00000168702 |
| Lung | USH24 | ENSG00000042781 |
| Lung | FLG | ENSG00000143631 |
| Lung | PCLO | ENSG00000186472 |
| Lung | PIK3CA | ENSG00000121879 |
| Lung | OBSCN | ENSG00000154358 |
| Lung | ZFHX4 | ENSG00000091656 |
| Lung | MUC4 | ENSG00000145113 |
| Lung | DNAH5 | ENSG00000039139 |
| Lung | CSMD1 | ENSG00000183117 |
| Lung | FAT4 | ENSG00000196159 |
| Lung | FAT3 | ENSG00000165323 |
| Lung | DST | ENSG00000151914 |
| Lung | XIRP2 | ENSG00000163092 |
| Lung | HMCN1 | ENSG00000143341 |
| Lung | KMT2D | ENSG00000167548 |
| Lung | RYR1 | ENSG00000196218 |
| Lung | SPTA1 | ENSG00000163554 |
| Lung | MUC17 | ENSG00000169876 |
| Lung | APOB | ENSG00000084674 |
| Lung | RYR3 | ENSG00000198838 |
| Lung | MACF1 | ENSG00000127603 |
| Lung | KRAS | ENSG00000133703 |
| Lung | PCDH15 | ENSG00000150275 |
| Lung | NEB | ENSG00000183091 |
| Lung | ADGRY1 | ENSG00000164199 |
| Lung | AHNAK2 | ENSG00000185567 |
| Lung | LRP2 | ENSG00000081479 |
| Lung | KMT2C | ENSG00000055609 |
| Lung | DNAH9 | ENSG00000007174 |
| Lung | PTEN | ENSG00000171862 |
| Lung | MUC5B | ENSG00000117983 |
| Lung | DNAH8 | ENSG00000124721 |
| Lung | ABCA13 | ENSG00000179869 |
| Lung | CSMD2 | ENSG00000121904 |
| Lung | DMD | ENSG00000198947 |
| Lung | DNAH11 | ENSG00000105877 |
| Lung | PKHD1L1 | ENSG00000205038 |
| Lung | ARID1A | ENSG00000117713 |
| Lung | SYNE2 | ENSG00000054654 |
| Lung | FAT1 | ENSG00000083857 |
| Lung | DNAH7 | ENSG00000118997 |
| Lung | ANK2 | ENSG00000145362 |
| Lung | DNAH3 | ENSG00000158486 |
| Lung | APC | ENSG00000134982 |
| Lung | PKHD1 | ENSG00000170927 |
| Lung | CACNA1E | ENSG00000198216 |
| Lung | COL6A3 | ENSG00000163359 |
| Lung | RELN | ENSG00000189056 |
| Lung | HYDIN | ENSG00000157423 |
| Lung | AHNAK | ENSG00000124942 |
| Lung | BRAF | ENSG00000157764 |
| Lung | CUBN | ENSG00000107611 |
| Lung | IGHG1 | ENSG00000211896 |
| Lung | FAM135B | ENSG00000147724 |
| Lung | NPAP1 | ENSG00000185823 |
| Lung | NAV3 | ENSG00000067798 |
| Lung | ZNFS36 | ENSG00000198597 |
| Lung | COL11A1 | ENSG00000060718 |
| Lung | ANK3 | ENSG00000151150 |
| Lung | FCGBP | ENSG00000275395 |
| Lung | DNAH17 | ENSG00000187775 |
| Lung | PAPPA2 | ENSG00000116183 |
| Lung | TENM1 | ENSG00000009694 |
| Lung | NRXN1 | ENSG00000179915 |
| Lung | ATRX | ENSG00000085224 |
| Lung | SSPO | ENSG00000197558 |
| Lung | DNAH10 | ENSG00000197653 |
| Lung | HERC2 | ENSG00000128731 |
| Lung | NF1 | ENSG00000196712 |
| Lung | MXRA5 | ENSG00000101825 |
| Lung | DSCAM | ENSG00000171587 |
| Lung | LAMA1 | ENSG00000101680 |
| Lung | SI | ENSG00000090402 |
| Lung | SACS | ENSG00000151835 |
| Lung | FAT2 | ENSG00000086570 |
| Lung | RNF213 | ENSG00000173821 |
| Lung | DCHS2 | ENSG00000197410 |
| Lung | RP1 | ENSG00000104237 |
| Lung | LRP1 | ENSG00000123384 |
| Lung | RIMS2 | ENSG00000176406 |
| Lung | PLEC | ENSG00000178209 |
| Lung | HUWE1 | ENSG00000086758 |
| Lung | FMN2 | ENSG00000155816 |
| Lung | PLXNA4 | ENSG00000221866 |
| Lung | PCDH11X | ENSG00000102290 |
| Lung | DNAH2 | ENSG00000183914 |
| Lung | FBN2 | ENSG00000138829 |
| Lung | ZFHX3 | ENSG00000140836 |
| Lung | PTPRT | ENSG00000196090 |
| Lung | HRNR | ENSG00000197915 |
| Lung | KIAA1109 | ENSG00000138688 |
| Lung | COL22A1 | ENSG00000169436 |
| Lung | PTPRD | ENSG00000153707 |

TABLE 15B

| Indication | Cell Lines |
|---|---|
| Glioblastoma | U-138 MG |
| Glioblastoma | LN-229 |
| Glioblastoma | U-87 MG |
| Glioblastoma | T98G |
| Glioblastoma | M059K |
| Glioblastoma | U-118 MG |
| Glioblastoma | LN-18 |
| Glioblastoma | DBTRG-05MG |
| Glioblastoma | A-172 |
| Glioblastoma | M059J |
| Glioblastoma | B104-1-1 |
| Glioblastoma | 9L/lacZ |
| Pancreatic | SW1990 |
| Pancreatic | SU.86.86 |
| Pancreatic | MIA-PaCa-2 |
| Pancreatic | CFPAC-1 |
| Pancreatic | HPAF-II |
| Pancreatic | SW 1990 |
| Pancreatic | Capan-1 |
| Pancreatic | MIA PaCa-2 |
| Pancreatic | BxPC-3 |
| Pancreatic | PANC-1 Ecadherin EmGFP |
| Pancreatic | LTPA |
| Pancreatic | HPAC |
| Pancreatic | AsPC-1 |
| Pancreatic | 1116-NS-19-9 |
| Pancreatic | Panc 10.05 |
| Pancreatic | Capan-2 |
| Lung | 201T |
| Lung | A549 |
| Lung | ABC-1 |
| Lung | Calu-3 |
| Lung | Calu-6 |
| Lung | COR-L105 |
| Lung | EKVX |
| Lung | EMC-BAC-1 |
| Lung | EMC-BAC-2 |
| Lung | H3255 |
| Lung | HCC-44 |
| Lung | HCC-78 |
| Lung | HCC-827 |
| Lung | LC-2-ad |
| Lung | LXF-289 |
| Lung | NCI-H1355 |
| Lung | NCI-H1395 |
| Lung | NCI-H1435 |
| Lung | NCI-H1563 |
| Lung | NCI-H1568 |
| Lung | NCI-H1573 |
| Lung | NCI-H1623 |
| Lung | NCI-H1648 |
| Lung | NCI-H1650 |
| Lung | NCI-H1651 |
| Lung | NCI-H1666 |
| Lung | NCI-H1693 |
| Lung | NCI-H1703 |
| Lung | NCI-H1734 |
| Lung | NCI-H1755 |
| Lung | NCI-H1781 |
| Lung | NCI-H1792 |
| Lung | NCI-H1793 |
| Lung | NCI-H1838 |
| Lung | NCI-H1944 |
| Lung | NCI-H1975 |
| Lung | NCI-H1993 |
| Lung | NCI-H2009 |
| Lung | NCI-H2023 |
| Lung | NCI-H2030 |
| Lung | NCI-H2085 |
| Lung | NCI-H2087 |
| Lung | NCI-H2122 |
| Lung | NCI-H2228 |
| Lung | NCI-H2291 |
| Lung | NCI-H23 |
| Lung | NCI-H2342 |
| Lung | NCI-H2347 |
| Lung | NCI-H2405 |
| Lung | NCI-H292 |
| Lung | NCI-H3122 |
| Lung | NCI-H322M |
| Lung | NCI-H358 |
| Lung | NCI-H441 |
| Lung | NCI-H522 |
| Lung | NCI-H596 |
| Lung | NCI-H650 |
| Lung | NCI-H838 |
| Lung | PC-14 |
| Lung | RERF-LC-KJ |
| Lung | RERF-LC-MS |
| Lung | SK-LU-1 |
| Lung | SW1573 |
| Lung | NCI-H720 |
| Lung | NCI-H727 |
| Lung | NCI-H835 |
| Lung | UMC-11 |
| Lung | COR-L23 |
| Lung | HOP-92 |
| Lung | IA-LM |
| Lung | LCLC-103H |
| Lung | LCLC-97TM1 |
| Lung | LU-65 |
| Lung | LU-99A |
| Lung | NCI-H1155 |
| Lung | NCI-H1299 |
| Lung | NCI-H1581 |
| Lung | NCI-H1915 |
| Lung | NCI-H661 |
| Lung | NCI-H810 |
| Lung | A427 |
| Lung | BEN |
| Lung | CAL-12T |
| Lung | ChaGo-K-1 |
| Lung | HCC-366 |
| Lung | NCI-H1770 |
| Lung | NCI-H2110 |
| Lung | NCI-H2135 |
| Lung | NCI-H2172 |
| Lung | NCI-H2444 |
| Lung | NCI-H647 |
| Lung | EBC-1 |
| Lung | EPLC-272H |
| Lung | HARA |
| Lung | HCC-15 |
| Lung | KNS-62 |
| Lung | LC-1-sq |
| Lung | LK-2 |
| Lung | LOU-NH91 |
| Lung | NCI-H1869 |
| Lung | NCI-H2170 |
| Lung | NCI-H226 |
| Lung | NCI-H520 |
| Lung | RERF-LC-Sq1 |
| Lung | SK-MES-1 |
| Lung | SW900 |
| Lung | COR-L321 |
| Lung | COLO-668 |
| Lung | COR-L279 |
| Lung | COR-L303 |
| Lung | COR-L311 |
| Lung | COR-L32 |
| Lung | COR-L88 |
| Lung | CPC-N |
| Lung | DMS-114 |
| Lung | DMS-273 |
| Lung | DMS-53 |
| Lung | IST-SL1 |
| Lung | IST-SL2 |
| Lung | LB647-SCLC |
| Lung | LU-134-A |
| Lung | LU-135 |
| Lung | LU-139 |
| Lung | LU-165 |
| Lung | MS-1 |
| Lung | NCI-H1048 |
| Lung | NCI-H1092 |
| Lung | NCI-H1105 |
| Lung | NCI-H1341 |
| Lung | NCI-H1417 |
| Lung | NCI-H1436 |
| Lung | NCI-H146 |
| Lung | NCI-H1688 |
| Lung | NCI-H1694 |
| Lung | NCI-H1836 |
| Lung | NCI-H187 |
| Lung | NCI-H1876 |
| Lung | NCI-H196 |
| Lung | NCI-H1963 |
| Lung | NCI-H2029 |
| Lung | NCI-H2066 |
| Lung | NCI-H209 |
| Lung | NCI-H211 |
| Lung | NCI-H2141 |
| Lung | NCI-H2196 |
| Lung | NCI-H2227 |
| Lung | NCI-H250 |
| Lung | NCI-H345 |
| Lung | NCI-H378 |
| Lung | NCI-H446 |
| Lung | NCI-H510A |
| Lung | NCI-H524 |
| Lung | NCI-H526 |
| Lung | NCI-H64 |
| Lung | NCI-H69 |
| Lung | NCI-H748 |
| Lung | NCI-H82 |
| Lung | NCI-H841 |
| Lung | NCI-H847 |
| Lung | SBC-1 |
| Lung | SBC-3 |
| Lung | SBC-5 |
| Lung | H2369 |
| Lung | H2373 |
| Lung | H2461 |
| Lung | H2591 |
| Lung | H2595 |
| Lung | H2722 |
| Lung | H2731 |
| Lung | H2795 |
| Lung | H2803 |
| Lung | H2804 |
| Lung | H2810 |
| Lung | H2818 |
| Lung | H2869 |
| Lung | H290 |
| Lung | H513 |
| Lung | IST-MES1 |
| Lung | MPP-89 |
| Lung | MSTO-211H |
| Lung | NCI-H2052 |
| Lung | NCI-H2452 |
| Lung | NCI-H28 |
| Lung | DMS-79 |
| Lung | HOP-62 |
| Lung | NCI-H1437 |
| Lung | PC-3 [JPC-3] |
| Lung | NCI-H740 |
| Lung | COR-L95 |
| Lung | HCC-33 |
| Lung | NCI-H128 |
| Lung | NCI-H1304 |
| Lung | NCI-H2081 |
| Lung | NCI-H2171 |
| Lung | SHP-77 |
| Lung | SW1271 |
| Lung | VMRC-LCD |
| Lung | NCI-H460 |
| Lung | RERF-LC-FM |

Without wishing to be bound by theory, it is believed that the following protocols, as well as those detailed elsewhere herein, could be used on a variety of diseases including, but not limited to, viral and bacterial diseases, cancers, neurodegenerative diseases, and neuropsychiatric disorders.

### Affinity Purification Mass Spectrometry (AP-MS)

Plasmid Cloning

Sequences of interest are downloaded from Genbank and utilized to design 2×-Strep tagged expression constructs. Protein termini are analyzed for predicted acylation motifs, signal peptides, and transmembrane regions, and either the N- or C-terminus is chosen for tagging as appropriate. Finally, reading frames are codon optimized and cloned into pLVX-EF1alpha-IRES-Puro (Takara/Clontech) including a 5′ Kozak motif.

Transfection and Cell Harvest for Immunoprecipitation Experiments

For each affinity purification, ten million cells are transfected with up to 15 μg of individual expression constructs using PolyJet transfection reagent (SignaGen Laboratories) at a 1:3 μg:μl ratio of plasmid to transfection reagent based on manufacturer's protocol. After more than 38 hours, cells are dissociated at room temperature using 10 ml PBS without calcium and magnesium (D-PBS) with 10 mM EDTA for at least 5 minutes, pelleted by centrifugation at 200×g, at 4° C. for 5 minutes, washed with 10 ml D-PBS, pelleted once more and frozen on dry ice before storage at −80° C. for later immunoprecipitation analysis. For each protein, three independent biological replicates are prepared. Whole cell lysates are resolved on 4%-20% Criterion SDS-PAGE gels (Bio-Rad Laboratories) to assess Strep-tagged protein expression by immunoblotting using mouse anti-Strep tag antibody 34850 (QIAGEN) and anti-mouse HRP secondary antibody (BioRad).

Anti-Strep-Tag Affinity Purification

Frozen cell pellets are thawed on ice for 15-20 minutes and suspended in 1 ml Lysis Buffer, composed of 50 mM Tris-HCl, pH 7.4 at 4° C., 150 mM NaCl, 1 mM EDTA supplemented with 0.5% Nonidet P 40 Substitute (NP-40; Fluka Analytical) and cOmplete mini EDTA-free protease and PhosSTOP phosphatase inhibitor cocktails (Roche). Samples are then freeze-fractured by refreezing on dry ice for 10-20 minutes, then rethawed and incubated on a tube rotator for 30 minutes at 4° C. Debris is pelleted by centrifugation at 13,000×g, at 4° C. for 15 minutes. Up to 56 samples are arrayed into a 96-well Deepwell plate for affinity purification on the KingFisher Flex Purification System (Thermo Scientific) as follows: MagStrep “type3” beads (30 μl; IBA Lifesciences) are equilibrated twice with 1 ml Wash Buffer (IP Buffer supplemented with 0.05% NP-40) and incubated with 0.95 ml lysate for 2 hours. Beads are washed three times with 1 ml Wash Buffer and then once with 1 ml IP Buffer. Beads are released into 75 μl Denaturation-Reduction Buffer (2 M urea, 50 mM Tris-HCl pH 8.0, 1 mM DTT) in advance of on-bead digestion. All automated protocol steps are performed at 4° C. using the slow mix speed and the following mix times: 30 seconds for equilibration/wash steps, 2 hours for binding, and 1 minute for final bead release. Three 10 second bead collection times are used between all steps.

On-Bead Digestion for Affinity Purification

Bead-bound proteins are denatured and reduced at 37° C. for 30 minutes, alkylated in the dark with 3 mM iodoacetamide for 45 minutes at room temperature, and quenched with 3 mM DTT for 10 minutes. To offset evaporation, 22.5 μl 50 mM Tris-HCl, pH 8.0 is added prior to trypsin digestion. Proteins are then incubated at 37° C., initially for 4 hours with 1.5 μl trypsin (0.5 μg/μl; Promega) and then another 1-2 hours with 0.5 μl additional trypsin. All steps are performed with constant shaking at 1,100 rpm on a ThermoMixer C incubator. Resulting peptides are combined with 50 μl 50 mM Tris-HCl, pH 8.0 used to rinse beads and acidified with trifluoroacetic acid (0.5% final, pH<2.0). Acidified peptides are desalted for MS analysis using a BioPureSPE Mini 96-Well Plate (20 mg PROTO 300 C18; The Nest Group, Inc.) according to standard protocols.

Mass Spectrometry Operation and Peptide Search

Samples are re-suspended in 4% formic acid, 2% acetonitrile solution, and separated by a reversed-phase gradient over a nanoflow C18 column (Dr. Maisch). HPLC buffer A is composed of 0.1% formic acid, and HPLC buffer B was composed of 80% acetonitrile in formic acid. Peptides are eluted by a linear gradient from 7 to 36% B over the course of 52 min, after which the column is washed with 95% B, and re-equilibrated at 2% B. Each sample is directly injected via a Easy-nLC 1200 (Thermo Fisher Scientific) into a Q-Exactive Plus mass spectrometer (Thermo Fisher Scientific) and analyzed with a 75 minute acquisition, with all MS1 and MS2 spectra collected in the orbitrap; data is acquired using the Thermo software Xcalibur (4.2.47) and Tune (2.11 QF1 Build 3006). For all acquisitions, QCloud is used to control instrument longitudinal performance during the project (C. Chiva, R. Olivella, E. Bonis, G. Espadas, O. Pastor, A. Solé, E. Sabidó, QCloud: A cloud-based quality control system for mass spectrometry-based proteomics laboratories. PLoS One. 13, e0189209 (2018)). All proteomic data is searched against the human proteome, EGFP sequence, and the sequences of bait proteins using the default settings for MaxQuant (version 1.6.12.0) (J. Cox, M. Mann, MaxQuant enables high peptide identification rates, individualized p.p.b.-range mass accuracies and proteome-wide protein quantification. Nat. Biotechnol. 26, 1367-1372 (2008)). Detected peptides and proteins are filtered to 1% false discovery rate in MaxQuant.

### Scoring and Comparing Protein-Protein Interactions

High-Confidence Protein Interaction Scoring

Identified proteins are then subjected to protein-protein interaction scoring with SAINTexpress (version 3.6.3), MiST (https://github.com/kroganlab/mist), and compPASS (G. Teo, et al., SAINTexpress: improvements and additional features in Significance Analysis of INTeractome software. J. Proteomics. 100, 37-43 (2014); S. Jager, et al., Global landscape of HIV-human protein complexes. Nature. 481, 365-370 (2011); P. K. Jackson, Navigating the deubiquitinating proteome with a CompPASS. Cell. 138 (2009), pp. 222-224). A two-step filtering strategy is applied to determine the final list of reported interactors, which relies on two different scoring stringency cut-offs. In the first step, all protein interactions that fall above specific thresholds defined for MiST, compPASS, and/or SAINTexpress are chosen. For all proteins that fulfilled these criteria, information about the stable protein complexes that they participated in is extracted from the CORUM (M. Giurgiu, et al., CORUM: the comprehensive resource of mammalian protein complexes-2019. Nucleic Acids Res. 47, D559-D563 (2019)) database of known protein complexes. In the second step, the stringency is relaxed, and additional interactors that formed complexes with interactors determined in filtering step 1 are recovered. Proteins that fulfilled filtering criteria in either step 1 or step 2 are considered to be high-confidence protein-protein interactions (HC-PPIs).

Protein Protein Interaction Scoring: MiST

The MiST score is a weighted sum of three features: (1) normalized protein abundance measured by peak intensities, spectral counts, or unique number of peptide per protein (abundance); (2) invariability of abundance over replicated experiments (reproducibility); and (3) a measure of how unique a bait-prey pair is compared to all other baits (specificity). The weights of the three features are configurable in three different ways: first, pre-configured fixed weights can be used; second, they can be trained de novo on a custom list of trusted bait-prey pairs identified in the data set; lastly, a principal component analysis (PCA) can be run to assign the feature weights according their contribution to the variance in the data set.

Specifically, the amount of prey i interacting with bait b is quantified using modified SI_{N} score that is computed from a protein intensity I_{b,i} (not spectral counts as in the original design), total protein intensities of N number of preys observed from a single pull-down experiment is:

$${\sum }_{i = 1}^{N} {I}_{b , i} .$$

The length (number of residues) of the identified prey, Lᵢ, is as follows:

$$\text{?}$$

$$\text{?} \text{indicates text missing or illegible when filed}$$

The quantity Q_{b,i,r} of bait-prey pair b, i in a replica r is defined as SI_{N} score of b, i pair normalized by a sum of SI_{N} scores of all preys from a given pull-down experiment r as:

$${Q}_{b , i , r} = \frac{{SI}_{N ; b , i , r}}{{\sum }_{i = 1}^{N} {SI}_{N ; b , i , r}} .$$

Next, the three features used to define the biological relevance score are calculated as follows. The first feature, the abundance, A_{b,i}, of a given bait-prey pair i,b, is defined as the mean of the bait-prey quantities Q_{b,i,r} over all NR number of replicas:

$${A}_{b , i} = \frac{{\sum }_{r = 1}^{N \text{?}} {Q}_{b , i , r}}{{N}_{R}} .$$

$$\text{?} \text{indicates text missing or illegible when filed}$$

The second feature, the reproducibility, R_{b,i}, of a given bait-prey pair b,i, is defined as the normalized entropy of the vector Q_{b,i}:

$${R}_{b , i} + \frac{{\sum }_{r = 1}^{N \text{?}} {Q}_{b , i , r} \cdot log ( {Q}_{b , i , r} )}{{{log}_{2} ( {N}_{R} )}^{- 1}} .$$

$$\text{?} \text{indicates text missing or illegible when filed}$$

The third feature, the specificity, S_{b,i}, of a given bait-prey pair b, i, is defined as the proportion of the abundance of prey i compared to the abundances of prey i for the other N_{B} number of baits:

$${S}_{b , i} = \frac{{A}_{b , i}}{{\sum }_{b = 1}^{N \text{?}} {A}_{b , i}} .$$

$$\text{?} \text{indicates text missing or illegible when filed}$$

Optionally, MiST can exclude consideration of specificity for baits that are expected to bind similar preys (based on either manual annotation or clustering of pull-downs). The three features are combined into a single composite score (the MiST score) by maximizing the variance in the three features space using the standard principal component analysis (PCA), as implemented in the MDP toolkit.

Protein Protein Interaction Scoring: CompPASS

CompPASS is an acronym for Comparative Proteomic Analysis Software Suite. It relies on an unbiased comparative approach for identifying high-confidence candidate interacting proteins (HCIPs for short) from the hundreds of proteins typically identified in IP-MS/MS experiments. There are several scoring metrics calculated as part of comPASS: The Z-score, the S-score, the D-score, and the WD-score. The S-score, D-score, and WD-score were all developed empirically based on their ability to effectively discriminate known interactors from known background proteins. Each score has advantages and disadvantages, and each are used to assess distinct aspects of the dataset. However, the primary score use to determine the high-confidence protein-protein interaction dataset is the WD-score. Typically, the top 5% of the WD-score scores are taken (more information under “Determining Thresholds”).

The Z-Score. The first score is the conventional Z-score, which determines the number of standard deviations away from the mean (Eq. 1) at which a measurement lies (Eq. 2). In Eq. 1 & 2 X is the TSC, i is the bait number, j is the interactor, n denotes which interactor is being considered, k is the total number of baits, and s is the standard deviation of the TSC mean.

$$\begin{matrix}{\overline{x}}_{j} = \frac{{\sum }_{\text{?}}^{\text{?}} {x}_{i , j}}{k} & \text{?} n = 1 , 2 , … m\end{matrix} \qquad ( Eq . 1 )$$

$${z}_{i , j} = \frac{{x}_{i , j} - {\overline{x}}_{j}}{{σ}_{j}} \qquad ( Eq . 2 )$$

$$\text{?} \text{indicates text missing or illegible when filed}$$

Each interactor for each bait has a Z-score calculated and therefore, the same interactor will have a different Z-score depending on the bait (assuming the TSC is different when identified for that bait). Although the Z-score can effectively identify interactors who's TSC is significantly different from the mean, if an interactor is unique (found in association with only 1 bait), then it fails to discriminate between interactors with a single TSC (“one hit wonders”) and another that may have 20 TSC or 50 TSC, etc. In this way, the Z-score will tend to upweight unique proteins, no matter their abundance. This can be dangerous since the stochastic nature of data-dependent acquisition mass spectrometry leads to spurious identification of proteins. These would be assigned the maximal Z-score as they would be unique, however they likely do not represent bona fide interactors.

The S-Score. The next score is the S-score which incorporates the frequency of the observed interactor and its' abundance (TSC). Both the D- and WD-scores are based on the S-score, sharing the same fundamental formulation, but have additional terms that add increasing resolving power. The S-score (Eq. 3) is essentially a uniqueness and abundance measurement.

$$\begin{matrix}{S}_{i , j} = \sqrt{( \frac{k}{{\sum }_{\text{?}}^{\text{?}} \text{?}} ) {x}_{i , j}} ; & {f}_{i , j} = \{ \begin{matrix}1 \text{?} {x}_{i , j} > 0 \\ {x}_{i , j}\end{matrix}\end{matrix} \qquad ( Eq . 3 )$$

$$\text{?} \text{indicates text missing or illegible when filed}$$

In Eq. 3, the variables are the same as for Eq. 1 & 2. f is a term which is 0 or 1 depending on whether or not the interacting protein is found in a given bait. Placed in the summation across all baits, it is a counting term and therefore, k/Sf is the inverse ratio (or frequency) of this interactor across all baits. The smaller f the larger this value becomes and thus upweights interactors that are rare. The term X_{i,j} is the TSC for interactor j from bait i and therefore multiplying by this value scales the S-score with increasing interactor TSC—this provides a higher score to interactors having high TSC and are therefore more abundant and less likely to be stochastically sampled. Although increasing the resolution above using the Z-score alone (the S-score can discriminate between unique one hit wonders and unique interactors with high TSC), the S-score will give its highest values to interactors that very rare and can lead to one hit wonders being scored among the top proteins. However, with a stringent cut-off value, the S-score reliably identifies HCIPs and bona fide interacting proteins but at this level, is prone to miss lower abundant likely interacting proteins. In order to address this limitation, the S-score is modified to take into account the reproducibility of the interactor for a given bait—a quantity that can be determined as a result of performing duplicate mass spectrometry runs. After adding this modification, the S-score becomes the D-score (Eq. 4).

The D-Score. The D-score is fundamentally the same as the S-score except with an added power term to take into account the reproducibility of the interaction. The term p can either be 1 (if the interactor was found in 1 of 2 duplicate runs) or 2 (if the interactor was found in both duplicate runs).

$$\begin{matrix}{D}_{\text{?}} = \sqrt{{( \frac{k}{{\sum }_{\text{?}}^{\text{?}} \text{?}} )}^{P} {x}_{i , j}} ; & \begin{matrix}{f}_{i , j} = \{ \begin{matrix}1 \text{?} {x}_{i , j} > 0 \\ {x}_{i , j}\end{matrix} \\ p = \begin{matrix}\text{?} \\ \text{?}\end{matrix}\end{matrix}\end{matrix} \qquad ( Eq . 4 )$$

$$\text{?} \text{indicates text missing or illegible when filed}$$

If p is 1 (the interactor was found in 1 of 2 duplicates) then the D-score is the same as the S-score. Adding the reproducibility term now allows for better discrimination between a true one hit wonder (a protein found with 1 peptide in a single run, not in the duplicate) which is likely a false positive versus a true interactor with low (even 1) TSC that is found in both duplicate runs. Although powerful in its ability to delineate HCIPs from background proteins, the D-score still relies heavily on the frequency term, k/Sf and will thus assign lower scores to more frequently observed proteins. In the vast majority of the cases, this is of course a good thing since these proteins are more than likely background. However, in the event that a canonical background protein is a bona fide interactor for a specific bait, its D-score would likely be too low for passing the D-score threshold (discussed below) and would not be considered a HCIP. Another example pertains to CompPASS analysis of baits from within the same biological network or pathway. In the case of the Dub Project, most of these proteins do not share interactors as this analysis is performed across a protein family—in which case the D-score works very well. However, sometimes baits do share interactors as these proteins are part of the same biological pathway and determining these share interactors (and hence the connections among these proteins) is critical for a reliable assessment of the pathway. In these cases, the D-score works fairly well for most interactors, however it can downweigh very commonly found bona fide interactors (especially when these interactors have low TSC). To address this limitation, a weighting factor was designed to be added into the D-score and thus created the WD-score (or Weighted D-score; Eq. 5).

The WD-Score. Upon examination of frequently observed proteins (considered background) that are either known not to be a bona fide interactor for any bait and those that are known to be true interactors for a subset of baits, it is found that the distributions of the TSC for these groups vary in a correlated manner. In the first case, where these “background” proteins are never true interactors, the standard deviation of the TSC (s_{TSC}) is smaller than that of the latter case (“background” proteins that are known to be true interactors for specific baits). This occurs since real background protein abundance is mainly determined by the amount of resin used in the IP whereas in the case of a background protein becoming a true interactor, its TSC then rises far above this consistent level (and thus cause s_{TSC} to increase. In fact, when s_{TSC} is systematically examined across all proteins found in >50% of the IP-MS/MS datasets, the proteins that are known to be real interactors for specific baits are found to have a s_{TSC} that is >100% of the TSC mean for that protein across all IPs. Therefore, a weight factor term is introduced as wⱼ and is essentially the s_{TSC}/TSC mean for interactor j (shown below).

$${WD}_{i , j} = \sqrt{{( \frac{k}{{\sum }_{\text{?}}^{\text{?}} \text{?}} {ω}_{j} )}^{P} {x}_{i , j}} \qquad ( Eq . 5 )$$

$${ω}_{j} = ( \frac{{σ}_{j}}{{\overline{x}}_{j}} ) , \qquad \begin{matrix}{\overline{x}}_{j} = \frac{{\sum }_{\text{?}}^{\text{?}} {x}_{i , j}}{k} & \text{?} n = 1 , 2 , … m ,\end{matrix} \qquad \begin{matrix}if {ω}_{j} \text{?} 1 \text{?} {ω}_{j} = 1 \\ if {ω}_{j} \text{?} 1 \text{?} {ω}_{j} = {ω}_{j}\end{matrix}$$

$$\begin{matrix}{f}_{i , j} = \{ \begin{matrix}1 ; {x}_{i , j} > 0 \\ {x}_{i , j}\end{matrix} & p = \begin{matrix}number of \text{?} in \\ which the \text{?} is present\end{matrix}\end{matrix}$$

$$\text{?} \text{indicates text missing or illegible when filed}$$

The weight factor, wⱼ, is added as a multiplicative factor to the frequency term in order to offset this low value for interactors that are found frequently across baits but will only be >1 if the conditions in Eq. 5 are met. If these conditions are not met, then oⱼ is set to 1 and the WD-score is the same as the D-score. In this way, only if a frequent interactor displays the observed characteristics of a true interactor will its score increase due to the weight factor.

To determine score thresholds for determining high-confidence protein-protein interactions, randomly generated simulated run data are compared against. In order to create simulated random runs, the data from actual experiments is first used to create the proteome observed from the experiments. To do this, each protein is represented by its TSC from each run—in other words, if a protein is found with a total of 450 TSC summed across all real runs, then it is represented 450 times. Simulated runs are then created by randomly drawing from this “experimental proteome” until 300 proteins are selected and the total TSC for the simulated run is 1500 (these are the average values found across the actual experiments). Next, scores are calculated for the random runs to determine the distributions of the scores for random data. Finally, for each score, the corresponding value above which 5% of the random data lies is found, and that value taken to be that score's threshold. Although 5% of the random data is above this threshold value, an examination of the TSC distribution for these random data is expected to show that >99% have TSC<4. Therefore, although there are false positive HCIPs in real datasets, this distribution can now be used to assign a p-value for proteins passing the score thresholds. In this way, an argument can be made that a protein passing a score threshold and found to have high enough TSC (reflected in the p-value) is very likely to be a real interactor. A suitable approximation for this above described method is to simply take the minimal value of the top 5% of the scores for each metric and set that value to be the threshold for that score.

Protein-Protein Interaction Scoring: SAINT

The aim of SAINT is to convert the label free quantification (spectral count Xᵢⱼ) for a prey protein i identified in a purification of bait j into the probability of true interaction between the two proteins, P(True|Xᵢⱼ). The spectral counts for each prey-bait pair are modeled with a mixture distribution of two components representing true and false interactions. Note that these distributions are specific to each bait-prey pair. The parameters for true and false distributions, P(Xᵢⱼ|True) and P(Xᵢⱼ|False), and the prior probability π_{T} of true interactions in the dataset, are inferred from the spectral counts for all interactions involving prey i and bait j. SAINT normalizes spectral counts to the length of the proteins and to the total number of spectra in the purification.

The spectral counts for prey i in purification with bait j are considered to be either from a Poisson distribution representing true interaction (with mean count λᵢⱼ) or from a Poisson distribution representing false interaction (with mean count κᵢⱼ. In the form of probability distribution, the following formula is written:

P(Xᵢⱼ|*)=π_{T}P(Xᵢⱼ|λᵢⱼ)+(I−π_{T})P(Xᵢⱼ|κᵢⱼ) (1)

where π_{T} is the proportion of true interactions in the data, and dot notation represents all relevant model parameters estimated from the data (here, specifically for the pair of prey i and bait j). The individual bait-prey interaction parameters λᵢⱼ and κᵢⱼ are estimated from joint modeling of the entire bait-prey association matrix, with the probability distribution (likelihood) of the form P(X|)=Π_{i,j}P(Xᵢⱼ|). The proportion π_{T} is also estimated from the model, which relies on latent variables in the sampling algorithm (see below).

When at least three control purifications are available, and assuming that the control purifications provide a robust representation of nonspecific interactors, the parameter κᵢⱼ can be estimated from spectral counts for prey i observed in the negative controls. This is equivalent to assuming

P(Xᵢⱼ|*)=π_{i,j;j∈E}(π_{T}P(Xᵢⱼ|λᵢⱼ)+(1−π_{T})P(Xᵢⱼ|κᵢⱼ))×π_{i,j,j∈C}(P(Xᵢⱼ|κᵢⱼ)) (2)

where E and C denote the group of experimental purifications and the group of negative controls, respectively. This leads to a semi-supervised mixture model in the sense that there is a fixed assignment to false interaction distribution for negative controls. As negative controls guarantee sufficient information for inferring model parameters for false interaction distributions, Bayesian nonparametric inference using Dirichlet process mixture priors can be used to derive the posterior distribution of protein-specific abundance parameters in the model. As a result, the mean parameters in the Poisson likelihood functions follow a nonparametric posterior distribution, allowing more flexible modeling at the proteome level. Under this setting, all model parameters are estimated from an efficient Markov chain Monte Carlo algorithm.

To elaborate on the two distributions, the mean parameter for each distribution is assumed to have the following form. For false interactions, it is assumed that spectral counts follow a Poisson distribution with mean count:

log(κᵢⱼ)=log(lᵢ)+log(cⱼ)+γ₀+μᵢ (3)

where lᵢ is the sequence length of prey i, and cⱼ is the bait coverage, the spectral count of the bait in its own purification experiment, γ₀ is the average abundance of all contaminants and μᵢ is prey i specific mean difference from γ₀. For true interactions, it is assumed that spectral counts follow a Poisson distribution with mean count:

log(λᵢⱼ)=log(lᵢ)+log(cⱼ)+β₀+α_{bj}+αₚᵢ (4)

where β₀ is the average abundance of prey proteins in those cases where they are true interactors of the bait, α_{bj} is bait j specific abundance factor and αₚᵢ is prey i specific abundance factor. In other words, the mean spectral count for a prey protein in a true interaction is calculated using a multiplicative model combining bait- and prey-specific abundance parameters. This formulation substantially reduces the number of parameters in the model, avoiding the need to estimate every λᵢⱼ separately.

For datasets without negative control purifications, the mixture component distributions for true and false interactions have to be identified solely from experimental (non-control) purifications. In this case, a user-specified threshold is applied to divide preys into high-frequency and low-frequency groups, denoted as Yᵢ=1 or 0 if prey i belongs to the high- or low-frequency group, respectively. An arbitrary 20% threshold is applied in the case of the DUB dataset; however, the results are not expected to be very sensitive to the choice of the threshold. For preys in the high frequency group, the model considers spectral counts for the observed prey proteins (ignoring zero count data, which represent the absence of protein identification), as there are sufficient data to estimate distribution parameters. In the low-frequency group, non-detection of a prey is included to help the separation of high-count from low-count hits. The entire mixture model can then be expressed as

P(Xᵢⱼ|*)=π_{i,j}(π_{T}P(Xᵢⱼ|λᵢⱼ)+(1−π_{T})P(Xᵢⱼ|κᵢⱼ))^{Z}^{ᵢⱼ} (5)

where Zᵢⱼ=1(Yᵢ=0)+1(Yᵢ=1,Xᵢⱼ>0) and the false and true interaction distributions are modeled by equations (3) and (4), respectively.

The posterior probability of a true interaction given the data is computed using Bayes rule

P(true|Xᵢⱼ)=TᵢⱼI(Tᵢⱼ+Fᵢⱼ) (6)

where Tᵢⱼ=π_{T}P(Xᵢⱼ|λᵢⱼ) and Fᵢⱼ=(1−π_{T}) P(Xᵢⱼ|κᵢⱼ). If there are replicate purifications for bait j, the final probability is computed as an average of individual probabilities over replicates. Note that one alternative approach is to compute the probability assuming conditional independence over replicates, that is, Π_{k∈j}P(Xᵢⱼₖ|λᵢⱼₖ) and Π_{k∈j}P(Xᵢⱼₖ|κᵢⱼₖ) for true and false interactions, with additional index k denoting replicates for bait j. Unlike average probability, this probability puts less emphasis on the degree of reproducibility, and thus may be more appropriate in datasets where replicate analysis of the same bait is performed using different experimental conditions (for example, purifications using different affinity tags) to increase the coverage of the interactome.

When probabilities have been calculated for all interaction partners, the Bayesian false discovery rate (FDR) can be estimated from the posterior probabilities as follows. For each probability threshold p*, the Bayesian FDR is approximated by

FDR(p*)=(Σₖ1(pₖ≥p*)(1−pₖ))/(Σₖ1(pₖ≥p*)) (7)

where pₖ is the posterior probability of true interaction of protein pair k. The output from SAINT allows the user to select a probability threshold to filter the data to achieve the desired FDR.

Comparing Protein Interactions Using Hierarchical Clustering

Hierarchical clustering is performed on interactions for distinct but related proteins, including viral proteins, cancer proteins, or proteins from other diseases, which are hereout simply referred to as “conditions.” First, protein interactions that pass the master threshold (defined in “High-confidence protein interaction scoring” section above) in at least one condition are assembled. New interaction scores (K) are created by taking the average of several interaction scores. This is done to provide a single score that captures the benefits from each scoring method. Clustering is then done using this new Interaction Score (K). Clustering is performed using the ComplexHeatmap package in R, using the “average” clustering method and “euclidean” distance metric. K-means clustering is applied to capture all possible combinations of interaction patterns between conditions.

Differential Interaction Score (DIS) Analysis

To compare PPIs across conditions (i.e., cell lines, viruses, diseases), a method for calculating a differential interaction score (DIS) was developed, and a corresponding false discovery rate (FDR) can be calculated using AP-MS data across multiple conditions. This approach uses the SAINTexpress score (G. Teo, et al., SAINTexpress: improvements and additional features in Significance Analysis of INTeractome software. J. Proteomics. 100, 37-43 (2014)), which is the probability of a PPI being bonafide in a single condition. Here, S_{c}(b, p) is the SAINTexpress score of a specific PPI denoted as (b, p) in a condition c. Here, an example is provided using three distinct conditions, C1, C2, and C3. Given that PPIs are independent events across different conditions, the differential interaction score is calculated for each PPI (b, p) as the product of the probability of a PPI being present in two of the conditions but absent in the third for each PPI:

DIS_{A}(b,p)=S_{C1}(b,p)×S_{C2}(b,p)×[1−S_{C3}(b,p)]

This differential interaction score highlights PPIs that are strongly conserved across two of the conditions, but not shared by the third. Additionally, PPIs that are present in the one conditions, but depleted in the other two, can be highlighted as follows:

DIS_{B}(b,p)=[1−S_{C1}(b,p)]×[1−S_{C2}(b,p)]×S_{C3}(b,p)

These two DIS scores can be further merged to define a single score for each PPI, where if DIS_{A}>DIS_{B}, the DIS is assigned a positive (+) sign, while if DIS_{A}<DIS_{B}, the unified DIS is assigned a negative (−) sign. In this way, the DIS for each PPI is represented by a continuum, in which negative DIS scores represent PPIs depleted in two of the three conditions, while positive DIS scores represent PPIs enriched in two of the three conditions. Additionally, for all differential interaction scores calculated, the Bayesian false discovery rate (BFDR) (G. Teo, G. Liu, J. Zhang, A. I. Nesvizhskii, A.-C. Gingras, H. Choi, SAINTexpress: improvements and additional features in Significance Analysis of INTeractome software. J. Proteomics. 100, 37-43 (2014)) estimates are also computed at all possible thresholds (p*) as follows:

$$F D R ( {p}^{*} ) = \frac{{\sum }_{i , i} ( 1 - D I S ( {p}_{i} , {p}_{j} ) ) \times I \{ D I S ( {p}_{i} , {p}_{j} ) > {p}^{*} \}}{{\sum }_{i , j} I \{ DIS ( {p}_{i} , {p}_{j} ) > {p}^{*} \}} ,$$

$$where I \{ A \} is 1 when A is True and 0 otherwise .$$

Note, while these scores are used here for comparison across 3 conditions, it can also be used more simply to compare between any two conditions. Such a comparison is calculated as follows where DIS_{1/2} results in PPIs specific to condition 1 have a positive DIS value, while PPIs specific to condition 2 results in a negative DIS value:

DIS_{C1/C2}(p₁,p₂)=S_{C1}(p₁,p₂)×(1−S_{C2}(p₁,p₂)) or

DIS_{C3/C2}(p₁,p₂)=S_{C3}(p₁,p₂)×(1−S_{C2}(p₁,p₂)) or

DIS_{C3/C1}(p₁,p₂)=S_{C3}(p₁,p₂)×(1−S_{C1}(p₁,p₂)).

### Genetic Perturbation Analysis

Network Generation and Visualization

Protein-protein interaction networks are generated in Cytoscape (P. Shannon, et al., Cytoscape: a software environment for integrated models of biomolecular interaction networks. Genome Res. 13, 2498-2504 (2003)) and subsequently annotated using Adobe Illustrator. Host-host physical interactions, protein complex definitions, and biological process groupings are derived from CORUM (M. Giurgiu, et al., CORUM: the comprehensive resource of mammalian protein complexes-2019. Nucleic Acids Res. 47, D559-D563 (2019)), Gene Ontology (biological process), and manually curated from literature sources. All networks are deposited in NDEx (R. T. Pillich, J. Chen, V. Rynkov, D. Welker, D. Pratt, NDEx: A Community Resource for Sharing and Publishing of Biological Networks. Methods Mol. Biol. 1558, 271-301 (2017)).

siRNA Library and Transfection into Human Cells

An OnTargetPlus siRNA SMARTpool library (Horizon Discovery) is purchased targeting proteins of interest. This library is arrayed in 96-well format, with each plate also including two non-targeting siRNAs as well as positive and negative controls. The siRNA library is transfected into cells using Lipofectamine RNAiMAX reagent (Thermo Fisher). Briefly, 6 pmoles of each siRNA pool are mixed with 0.25 μl RNAiMAX transfection reagent and OptiMEM (Thermo Fisher) in a total volume of 20 μl. After a 5 minute incubation period, the transfection mix is added to cells seeded in a 96-well format. 24 hours post-transfection, the cells are subjected to viral infection or drug treatment as warranted by the current investigation. Next, the cells are incubated for 72 hours to assess cell viability using the CellTiter-Glo luminescent viability assay according to the manufacturer's protocol (Promega). Luminescence is measured in a Tecan Infinity 2000 plate reader, and percentage viability calculated relative to untreated cells (100% viability) and cells lysed with 20% ethanol or 4% formalin (0% viability), included in each experiment.

Knockdown Validation with qRT-PCR in Human Cells

Gene-specific quantitative PCR primers targeting all genes represented in the OnTargetPlus library are purchased and arrayed in a 96-well format identical to that of the siRNA library (IDT). Cells treated with siRNA are lysed using the Luna® Cell Ready Lysis Module (New England Biolabs) following the manufacturer's protocol. The lysate is used directly for gene quantification by RT-qPCR with the Luna® Universal One-Step RT-qPCR Kit (New England Biolabs), using the gene-specific PCR primers and GAPDH as a housekeeping gene. The following cycling conditions are used in an Applied Biosystems QuantStudio 6 thermocycler: 55° C. for 10 minutes, 95° C. for 1 minute, and 40 cycles of 95° C. for 10 seconds, followed by 60° C. for 1 minute. The fold change in gene expression for each gene is derived using the 2^{-ΔΔCT}, 2 (Delta Delta CT) method (K. J. Livak, T. D. Schmittgen, Analysis of relative gene expression data using real-time quantitative PCR and the 2(-Delta Delta C(T)) Method. Methods. 25, 402-408 (2001)), normalized to the constitutively expressed housekeeping gene GAPDH. Relative changes are generated comparing the control siRNA knockdown transfected cells to the cells transfected with each siRNA.

sgRNA Selection and Synthesis for Cas9 Knockout Screen

sgRNAs are designed according to Synthego's multi-guide gene knockout (R. Stoner, T. Maures, D. Conant, Methods and systems for guide ma design and use. US Patent (2019), (available at https://patentimages. storage.googleapis. com/95/c7/43/3d48387ce0f116/US20190382797A1.p df)). Briefly, two or three sgRNAs are bioinformatically designed to work in a cooperative manner to generate small, knockout-causing, fragment deletions in early exons. These fragment deletions are larger than standard indels generated from single guides. The genomic repair patterns from a multi-guide approach are highly predictable based on the guide-spacing and design constraints to limit off-targets, resulting in a higher probability protein knockout phenotype. RNA oligonucleotides are chemically synthesized on Synthego solid-phase synthesis platform, using CPG solid support containing a universal linker. 5-Benzylthio-1H-tetrazole (BTT, 0.25 M solution in acetonitrile) is used for coupling, (3-((Dimethylamino-methylidene)amino)-3H-1,2,4-dithiazole-3-thione (DDTT, 0.1 M solution in pyridine)) is used for thiolation, dichloroacetic acid (DCA, 3% solution in toluene) is used for detritylation. Modified sgRNA are chemically synthesized to contain 2′-O-methyl analogs and 3′ phosphorothioate nucleotide interlinkages in the terminal three nucleotides at both 5′ and 3′ ends of the RNA molecule. After synthesis, oligonucleotides are subject to a series of deprotection steps, followed by purification by solid phase extraction (SPE). Purified oligonucleotides are analyzed by ESI-MS.

Arrayed Knockout Generation with Cas9-RNPs

For transfection into human cells, 10 pmol Streptococcus Pyogenes NLS-Sp.Cas9-NLS (SpCas9) nuclease (Aldevron; 9212) is combined with 30 pmol total synthetic sgRNA (10 pmol each sgRNA, Synthego) to form ribonucleoproteins (RNPs) in 20 μl total volume with SF Buffer (Lonza VSSC-2002) and allowed to complex at room temperature for 10 minutes. All cells are dissociated into single cells using TrypLE Express (Gibco), resuspended in culture media and counted. 100,000 cells per nucleofection reaction are pelleted by centrifugation at 200×g for 5 minutes. Following centrifugation, cells are resuspended in transfection buffer according to cell type and diluted to 2×10⁴ cells/μl. 5 μl of cell solution was added to preformed RNP solution and gently mixed. Nucleofections were performed on a Lonza HT 384-well nucleofector system (Lonza, #AAU-1001) using program CM-150 Immediately following nucleofection, each reaction is transferred to a tissue-culture treated 96-well plate containing 100 μl normal culture media and seeded at a density of 50,000 cells/well. Transfected cells are incubated following standard protocols.

Quantification of Arrayed Knockout Efficiency

Two days post-nucleofection, genomic DNA is extracted from cells using DNA QuickExtract (Lucigen, #QE09050). Briefly, cells are lysed by removal of the spent media followed by addition of 40 μl of QuickExtract solution to each well. Once the QuickExtract DNA Extraction Solution is added, the cells are scraped off the plate into the buffer. Following transfer to compatible plates, DNA extract is then incubated at 68° C. for 15 minutes followed by 95° C. for 10 minutes in a thermocycler before being stored for downstream analysis Amplicons for indel analysis are generated by PCR amplification with NEBNext polymerase (NEB, #M0541) or AmpliTaq Gold 360 polymerase (Thermo Fisher Scientific, #4398881) according to the manufacturer's protocol. The primers are designed to create amplicons between 400-800 bp, with both primers at least 100 bp distance from any of the sgRNA target sites. PCR products are cleaned-up and analyzed by Sanger sequencing (Genewiz). Sanger data files and sgRNA target sequences are input into Inference of CRISPR Edits (ICE) analysis (ice.synthego.com) to determine editing efficiency and to quantify generated indels (T. Hsiau, T. Maures, K. Waite, J. Yang, R. Kelso, K. Holden, R. Stoner, Inference of CRISPR Edits from Sanger Trace Data (2018), p. 251082). Percentage of alleles edited is expressed as an ice-d score. This score is a measure of how discordant the sanger trace is before vs. after the edit. It is a simple and robust estimate of editing efficiency in a pool, especially suited to highly disruptive editing techniques like multi-guide.

Identification of Essential Genes for siRNA and Cas9 Knockout Screen

Here, longitudinal imaging in human cells is used to assess cell viability. For benchmarking, relative cell viability is measured by CellTiter-Glo Luminescent Cell Viability Assay (Promega; G7571) as per manufacturer's instructions. Briefly, two passages post-nucleofection siRNA pools cultured in 96-well tissue-culture treated plates (Corning, #3595) are lysed in the CellTIter-Glo reagent, by removing spent media and adding 100 μl of the CellTiter-Glo reagent containing the CellTiter-Glo buffer and CellTiter-Glo Substrate. Cells are placed on an orbital shaker for 2 minutes on a SpectraMax iD5 (Molecular Devices) and then incubated in the dark at room temperature for 10 minutes. Completely lysed cells are pipette mixed and 25 μl are transferred to a 384-well assay plate (Corning, #3542). The luminescence is recorded on a SpectraMax iD5 (Molecular Devices) with an integration time of 0.25 seconds per well. Luminescence readings are all normalized to the without-sgRNA control condition.

To determine cell viability in Caco-2 knockouts, longitudinal imaging is used. All gene knockout pools are maintained for a minimum of six passages to determine the effect of loss of protein function on cell fitness prior to viral infection. Viability is determined through longitudinal imaging and automated image analysis using a Celigo Imaging Cytometer (Celigo). Each gene knockout pool is split in triplicate wells on separate plates. Every day, except the day of seeding, each well is scanned and analyzed using built in “Confluence” imaging parameters using auto-exposure and autofocus with an offset of −45 μm. Analysis is performed with standard settings except for an intensity threshold setting of 8. Confluency is averaged across 3 wells and plotted over time. Viability genes are determined as pools that are less than 20% confluent 5 days post seeding following 6 passages. Genes deemed essential are excluded from the knockout screen.

Quantitative Analysis and Scoring of Knockdown and Knockout Library Screens

Assay readouts from genetic perturbation screens are processed using the RNAither package (https://www.bioconductor.org/packages/release/bioc/html/RNAither.html) in the statistical computing environment R. The two datasets are normalized separately, using the following method. The readouts are first log transformed (natural logarithm), and robust Z-scores (using median and MAD “median absolute deviation” instead of mean and standard deviation) are then calculated for each 96-well plate separately. Z-scores of multiple replicates of the same perturbation are averaged into a final Z-score for presentation.

### Cryogenic Electron Microscopy (Cryo-EM)

Co-Expression and Purification of Protein Complexes

Protein components are coexpressed using a pET29-b(+) vector backbone where one protein is tag-less and one has an N-terminal 10×His-tag and SUMO-tag. LOBSTR E. coli cells are transformed and grown at 37° C. till O.D. (600 nm)=0.8 and the expression is induced at 37° C. with 1 mM IPTG for 4 hours. Frozen cell pellets are resuspended in 25 ml lysis buffer (200 mM NaCl, 50 mM Tris-HCl pH 8.0, 10% v/v glycerol, 2 mM MgCl₂) per liter cell culture, supplemented with cOmplete protease inhibitor tablets (Roche), 1 mM PMSF (Sigma), 100 μg/ml lysozyme (Sigma), 5 μg/ml DNaseI (Sigma), and then homogenized with an immersion blender (Cuisinart). Cells are lysed by 3× passage through an Emulsiflex C3 cell disruptor (Avestin) at −15,000 psi, and the lysate clarified by ultracentrifugation at 100,000×g for 30 minutes at 4° C. The supernatant is collected, supplemented with 20 mM imidazole, loaded into a gravity flow column containing Ni-NTA superflow resin (Qiagen), and rocked with the resin at 4° C. for 1 hour. After allowing the column to drain, resin is rinsed twice with 5 column volumes (cv) of wash buffer (150 mM KCl, 30 mM Tris-HCl pH 8.0, 10% v/v glycerol, 20 mM imidazole, 0.5 mM tris(hydroxypropyl)phosphine (THP, VWR)) supplemented with 2 mM ATP (Sigma) and 4 mM MgCl₂, then washed with 5 cv wash buffer with 40 mM imidazole. Resin is then rinsed with 5 cv Buffer A (50 mM KCl, 30 mM Tris-HCl pH 8.0, 5% glycerol, 0.5 mM THP) and protein is eluted with 2×2.5 cv Buffer A+300 mM imidazole. Elution fractions are combined, supplemented with Ulp1 protease, and rocked at 4° C. for 2 hours. Ulp1-digested Ni-NTA eluate is diluted 1:1 with additional Buffer A, loaded into a 50 ml Superloop, and applied to a MonoQ 10/100 column on an Äkta pure system (GE Healthcare) using 100% Buffer A, 0% Buffer B (1000 mM KCl, 30 mM Tris-HCl pH 8.0, 5% glycerol, 0.5 mM THP). The MonoQ column is washed with 0%-40% Buffer B gradient over 15 cv, peak fractions are analyzed by SDS-PAGE and the identity of the tagless protein and the other protein confirmed by intact protein mass spectrometry (Xevo G2-XS Mass Spectrometer, Waters). Peak fractions are concentrated using 10 kDa Amicon centrifugal filter (Millipore) and further purified by size exclusion chromatography using a Superdex 200 increase 10/300 GL column (GE healthcare) in buffer containing 150 mM KCl, mM HEPES-NaOH pH 7.5, 0.5 mM THP. Peak fractions are used directly for cryo-EM grid preparation.

CryoEM Sample Preparation and Data Collection

Three μL of purified protein complex (12.5 μM) is added to a 400 mesh 1.2/1.3R Au Quantifoil grid previously glow discharged at 15 mA for 30 seconds. Blotting is performed with a blot force of 0 for 5 seconds at 4° C. and 100% humidity in a FEI Vitrobot Mark IV (ThermoFisher) prior to plunge freezing into liquid ethane. 1534 118-frame super-resolution movies are collected with a 3×3 image shift collection strategy at a nominal magnification of 105,000× (physical pixel size: 0.834 Å/pix) on a Titan Krios (ThermoFisher) equipped with a K3 camera and a Bioquantum energy filter (Gatan) set to a slit width of 20 eV. Collection dose rate is 8 e-/pixel/second for a total dose of 66 e-/Å2. Defocus range was −0.7 um to −2.4 um. Each collection is performed with semi-automated scripts in SerialEM (D. N. Mastronarde, Automated electron microscope tomography using robust prediction of specimen movements. J Struct. Biol. 152, 36-51 (2005)).

CryoEM Image Processing and Model Building

1534 movies are motion corrected using Motioncor2 (S. Q. Zheng, E. Palovcak, J.-P. Armache, K. A. Verba, Y. Cheng, D. A. Agard, MotionCor2: anisotropic correction of beam-induced motion for improved cryo-electron microscopy. Nat. Methods. 14, 331-332 (2017)) and dose-weighted summed micrographs are imported in cryosparc (v2.15.0). 1427 micrographs were curated based on CTF fit (better than 5 Å) from a patch CTF job. Template-based particle picking results in 2,805,121 particles and 1,616,691 particles are selected after 2D-classification. Five rounds of 3D-classification using multi-class ab-initio reconstruction and heterogenous refinement yields 178,373 particles. Homogenous refinement of these final particles leads to a 3.1 Å electron density map that is used for model building. The reconstruction is filtered by the masked FSC and sharpened with a b-factor of −145.

To build the model of the protein complex, crystal structures of orthologous proteins are used as a scaffold, and are fit into the cryoEM density as a rigid body in UCSF ChimeraX and then relaxed into the final density using Rosetta FastRelax mover in torsion space. This model, along with a BLAST alignment of the two sequences (S. F. Altschul, et al., Gapped BLAST and PSI-BLAST: a new generation of protein database search programs. Nucleic Acids Res. 25, 3389-3402 (1997)), is used as a starting point for manual building using COOT (P. Emsley, K. Cowtan, Coot: model-building tools for molecular graphics. Acta Crystallogr. D Biol. Crystallogr. 60, 2126-2132 (2004)). After initial building by hand the regions with poor density fit/geometry are iteratively rebuilt using Rosetta (R. Y.-R. Wang, et al., Automated structure refinement of macromolecular assemblies from cryo-EM maps using Rosetta. Elife. 5 (2016), doi:10.7554/eLife.17219). Final densities can be built using COOT, informed and facilitated by the predictions of the TargetP-2.0, MitoFates, and JPRED servers. The model of the protein complex is submitted to the Namdinator web server (R. T. Kidmose, et al., Namdinator—automatic molecular dynamics flexible fitting of structural models into cryo-EM and crystallography experimental maps. IUCrJ. 6, 526-531 (2019)) and further refined in ISOLDE 1.0 (T. I. Croll, ISOLDE: a physically realistic environment for model building into low-resolution electron-density maps. Acta Crystallogr D Struct Biol. 74, 519-530 (2018)) using the plugin for UCSF ChimeraX (T. D. Goddard, et al., UCSF ChimeraX: Meeting modern challenges in visualization and analysis. Protein Sci. 27, 14-25 (2018)). Final model B-factors are estimated using Rosetta. The model is validated using phenix.validation_cryoem (P. V. Afonine, B. P. Klaholz, N. W. Moriarty, B. K. Poon, O. V. Sobolev, T. C. Terwilliger, P. D. Adams, A. Urzhumtsev, New tools for the analysis and validation of cryo-EM maps and atomic models. Acta Crystallogr D Struct Biol. 74, 814-840 (2018)). Molecular interface residues between the proteins in the complex are analyzed using the PISA web server (E. Krissinel, K. Henrick, Inference of macromolecular assemblies from crystalline state. J. Mol. Biol. 372, 774-797 (2007)). Figures are prepared using UCSF ChimeraX.

### Determination of 3-Dimensional Structure of a Protein of Interest

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

A comparative overview of COVID-19, MERS and SARS: Review article. Int. J Surg. 81, 1-8 (2020).
J. H. Beigel, et al., Remdesivir for the treatment of Covid-19—preliminary report. N. Engl. J Med.
T. R. C. Group, The RECOVERY Collaborative Group, Dexamethasone in Hospitalized Patients with Covid-19—Preliminary Report. New England Journal of Medicine (2020), doi:10.1056/nejmoa2021436.
M. Becerra-Flores, T. Cardozo, SARS-CoV-2 viral spike G614 mutation exhibits higher case fatality rate. Int. J Clin. Pract. (2020), doi:10.1111/ijcp.13525.
D. E. Gordon, et al., SARS-CoV-2 protein interaction map reveals targets for drug repurposing. Nature (2020), doi:10.1038/s41586-020-2286-9.
G. Teo, et al., SAINTexpress: improvements and additional features in Significance Analysis of INTeractome software. J. Proteomics. 100, 37-43 (2014).
S. Jäger, et al., Global landscape of HIV-human protein complexes. Nature. 481, 365-370 (2011).
M. Giurgiu, et al., CORUM: the comprehensive resource of mammalian protein complexes-2019. Nucleic Acids Res. 47, D559-D563 (2019).
J. C. Young, et al., Molecular chaperones Hsp90 and Hsp70 deliver preproteins to the mitochondrial import receptor Tom70. Cell. 112, 41-50 (2003).
R. Lin, et al., Tom70 imports antiviral immunity to the mitochondria. Cell Res. 20, 971-973 (2010).
B. Wei, et al., Tom70 mediates Sendai virus-induced apoptosis on mitochondria. J. Virol. 89, 3804-3818 (2015).
A. M. Edmonson, et al., Characterization of a human import component of the mitochondrial outer membrane, TOMM70A. Cell Commun. Adhes. 9, 15-27 (2002).
J. Brix, et al., Differential recognition of preproteins by the purified cytosolic domains of the mitochondrial import receptors Tom20, Tom22, and Tom70. J. Biol. Chem. 272, 20730-20735 (1997).
J. Brix, et al., The mitochondrial import receptor Tom70: identification of a 25 kDa core domain with a specific binding site for preproteins. J. Mol. Biol. 303, 479-488 (2000).
R. D. Mills, et al., Domain organization of the monomeric form of the Tom70 mitochondrial import receptor. J. Mol. Biol. 388, 1043-1058 (2009).
S. D. Weeks, et al., X-ray Crystallographic Structure of Orf9b from SARS-CoV-2 (2020), doi:10.2210/pdb6z4u/pdb.
M. Bouhaddou, et al., The Global Phosphorylation Landscape of SARS-CoV-2 Infection. Cell (2020), doi:10.1016/j.cell.2020.06.034.
J. Li, et al., Molecular chaperone Hsp70/Hsp90 prepares the mitochondrial outer membrane translocon receptor Tom71 for preprotein loading. J Biol. Chem. 284, 23852-23859 (2009).
X.-Y. Liu, et al., Tom70 mediates activation of interferon regulatory factor 3 on mitochondria. Cell Res. 20, 994-1011 (2010).
B. E. Young, et al., Effects of a major deletion in the SARS-CoV-2 genome on the severity of infection and the inflammatory response: an observational cohort study. Lancet. 396, 603-611 (2020).
M. Zaretsky, et al., Directed evolution of a soluble human IL-17A receptor for the inhibition of psoriasis plaque formation in a mouse model. Chem. Biol. 20, 202-211 (2013).
Identification of a soluble isoform of human IL-17RA generated by alternative splicing. Cytokine. 64, 642-645 (2013).
Biological functions and therapeutic opportunities of soluble cytokine receptors. Cytokine Growth Factor Rev. (2020), doi:10.1016/j.cytogfr.2020.04.003.
M. Sammel, et al., Differences in Shedding of the Interleukin-11 Receptor by the Proteases ADAM9, ADAM10, ADAM17, Meprin α, Meprin β and MT1-MMP. Int. J. Mol. Sci. 20, 3677 (2019).
B. B. Sun, et al., Genomic atlas of the human plasma proteome. Nature. 558, 73-79 (2018).
Z. Zhu, et al., Causal associations between risk factors and common diseases inferred from GWAS summary data. Nat. Commun. 9, 224 (2018).
C. Huang, Y et al., The COVID-19 Host Genetics Initiative, a global initiative to elucidate the role of host genetic factors in susceptibility and severity of the SARS-CoV-2 virus pandemic. Eur. J Hum. Genet. 28, 715-718 (2020).
C. Amici, et al., Indomethacin has a potent antiviral activity against SARS coronavirus. Antivir. Ther. 11, 1021-1030 (2006).
P. R. Rosenbaum, D. B. Rubin, The central role of the propensity score in observational studies for causal effects. Biometrika. 70, 41-55 (1983).
C. Abate, et al., A structure-affinity and comparative molecular field analysis of sigma-2 (sigma2) receptor ligands. Cent. Nerv. Syst. Agents Med. Chem. 9, 246-257 (2009).
R. A. Glennon, Sigma receptor ligands and the use thereof. US Patent (2000), (available at https://patentimages.storage.googleapis.com/dc/36/68/73f4ccdac4c973/U.S. Pat. No. 6,057,371.pdf).

R. R. Matsumoto, B. Pouw, Correlation between neuroleptic binding to sigma(1) and sigma(2) receptors and acute dystonic reactions. Eur. J. Pharmacol. 401, 155-160 (2000).
M. Dold, et al., Haloperidol versus first-generation antipsychotics for the treatment of schizophrenia and other psychotic disorders. Cochrane Database Syst. Rev. 1, CD009831 (2015).
F. F. Moebius, et al., Pharmacological analysis of sterol delta8-delta7 isomerase proteins with [3H]ifenprodil. Mol. Pharmacol. 54, 591-598 (1998).
E. Gregori-Puigjané, et al., Identifying mechanism-of-action targets for drugs and probes. Proc. Natl. Acad. Sci. U.S.A 109, 11178-11183 (2012).
Z. Hubler, et al., Accumulation of 8,9-unsaturated sterols drives oligodendrocyte formation and remyelination. Nature. 560, 372-376 (2018).
F. F. Moebius, et al., High affinity of sigma 1-binding sites for sterol isomerization inhibitors: evidence for a pharmacological relationship with the yeast sterol C8-C7 isomerase. Br. J Pharmacol. 121, 1-6 (1997).
H.-W. Jiang, et al., SARS-CoV-2 Orf9b suppresses type I interferon responses by targeting TOM70. Cell. Mol. Immunol. 17, 998-1000 (2020).
Y. Perez-Riverol, et al., The PRIDE database and related tools and resources in 2019: improving support for quantification data. Nucleic Acids Res. 47, D442-D450 (2019).
J. J. Almagro Armenteros, et al., DeepLoc: prediction of protein subcellular localization using deep learning. Bioinformatics. 33, 3387-3395 (2017).
C. Chiva, et al., QCloud: A cloud-based quality control system for mass spectrometry-based proteomics laboratories. PLoS One. 13, e0189209 (2018).
J. Cox, M. Mann, MaxQuant enables high peptide identification rates, individualized p.p.b.-range mass accuracies and proteome-wide protein quantification. Nat. Biotechnol. 26, 1367-1372 (2008).
E. L. Huttlin, et al., The BioPlex Network: A Systematic Exploration of the Human Interactome. Cell. 162, 425-440 (2015).
G. Yu, et al., clusterProfiler: an R package for comparing biological themes among gene clusters. OMICS. 16, 284-287 (2012).
M. Remmert, et al., HHblits: lightning-fast iterative protein sequence searching by HMM-HMM alignment. Nat. Methods. 9, 173-175 (2011).
J. Yang, et al., Improved protein structure prediction using predicted interresidue orientations. Proc. Natl. Acad. Sci. U.S.A 117, 1496-1503 (2020).
Y. Zhai, et al., Insights into SARS-CoV transcription and replication from the structure of the nsp7-nsp8 hexadecamer. Nat. Struct. Mol. Biol. 12, 980-986 (2005).
A. Waterhouse, et al., SWISS-MODEL: homology modelling of protein structures and complexes. Nucleic Acids Res. 46, W296—W303 (2018).
J. Durairaj, et al., Geometricus Represents Protein Structures as Shape-mers Derived from Moment Invariants (2020), p. 2020.09.07.285569.
M. Akdel, et al., Caretta—A multiple protein structure alignment and feature extraction suite. Comput. Struct. Biotechnol. J. 18, 981-992 (2020).
P. Shannon, et al., Cytoscape: a software environment for integrated models of biomolecular interaction networks. Genome Res. 13, 2498-2504 (2003).
R. T. Pillich, et al., NDEx: A Community Resource for Sharing and Publishing of Biological Networks. Methods Mol. Biol. 1558, 271-301 (2017).
D. K. W. Chu, et al., Molecular Diagnosis of a Novel Coronavirus (2019-nCoV) Causing an Outbreak of Pneumonia. Clin. Chem. 66, 549-555 (2020).
K. J. Livak, T. D. Schmittgen, Analysis of relative gene expression data using real-time quantitative PCR and the 2(-Delta Delta C(T)) Method. Methods. 25, 402-408 (2001).
R. Stoner, T. Maures, D. Conant, Methods and systems for guide ma design and use. US Patent (2019).
T. Hsiau, et al., Inference of CRISPR Edits from Sanger Trace Data (2018), p. 251082.
A. S. Jureka, et al., Propagation, Inactivation, and Safety Testing of SARS-CoV-2. Viruses. 12 (2020), doi:10.3390/v12060622.
A. C. Y. Fan, et al., Hsp90 functions in the targeting and outer membrane translocation steps of Tom70-mediated mitochondrial import. J. Biol. Chem. 281, 33313-33324 (2006).
S. Backes, et al., Tom70 enhances mitochondrial preprotein import efficiency by binding to internal targeting sequences. J Cell Biol. 217, 1369-1382 (2018).
J. J. Almagro Armenteros, et al., Detecting sequence signals in targeting peptides using deep learning. Life Sci Alliance. 2 (2019), doi:10.26508/1sa.201900429.
Y. Fukasawa, et al., MitoFates: improved prediction of mitochondrial targeting sequences and their cleavage sites. Mol. Cell. Proteomics. 14, 1113-1126 (2015).
A. Drozdetskiy, et al., JPred4: a protein secondary structure prediction server. Nucleic Acids Res. 43, W389-94 (2015).
D. N. Mastronarde, Automated electron microscope tomography using robust prediction of specimen movements. J Struct. Biol. 152, 36-51 (2005).
S. Q. Zheng, et al., MotionCor2: anisotropic correction of beam-induced motion for improved cryo-electron microscopy. Nat. Methods. 14, 331-332 (2017).
S. F. Altschul, et al., Gapped BLAST and PSI-BLAST: a new generation of protein database search programs. Nucleic Acids Res. 25, 3389-3402 (1997).
P. Emsley, K. Cowtan, Coot: model-building tools for molecular graphics. Acta Crystallogr. D Biol. Crystallogr. 60, 2126-2132 (2004).
R. Y.-R. Wang, et al., Automated structure refinement of macromolecular assemblies from cryo-EM maps using Rosetta. Elife. 5 (2016), doi:10.7554/eLife.17219.
R. T. Kidmose, et al., Namdinator—automatic molecular dynamics flexible fitting of structural models into cryo-EM and crystallography experimental maps. IUCrJ. 6, 526-531 (2019).
T. I. Croll, ISOLDE: a physically realistic environment for model building into low-resolution electron-density maps. Acta Crystallogr D Struct Biol. 74, 519-530 (2018).
T. D. Goddard, et al., UCSF ChimeraX: Meeting modern challenges in visualization and analysis. Protein Sci. 27, 14-25 (2018).
P. V. Afonine, et al., A. Urzhumtsev, New tools for the analysis and validation of cryo-EM maps and atomic models. Acta Crystallogr D Struct Biol. 74, 814-840 (2018).
E. Krissinel, K. Henrick, Inference of macromolecular assemblies from crystalline state. J. Mol. Biol. 372, 774-797 (2007).
A. N. Honko, et al., Rapid Quantification and Neutralization Assays for Novel Coronavirus SARS-CoV-2 Using Avicel RC-591 Semi-Solid Overlay.
A. Sali, T. L. Blundell, Comparative protein modelling by satisfaction of spatial restraints. J. Mol. Biol. 234, 779-815 (1993).
T. Yamada, et al., Crystal structure and possible catalytic mechanism of microsomal prostaglandin E synthase type 2 (mPGES-2). J. Mol. Biol. 348, 1163-1176 (2005).
W. Yin, et al., Structural basis for inhibition of the RNA-dependent RNA polymerase from SARS-CoV-2 by remdesivir. Science. 368, 1499-1504 (2020).
D. Kozakov, et al., The ClusPro web server for protein-protein docking. Nat. Protoc. 12, 255-278 (2017).
B. G. Pierce, et al., ZDOCK server: interactive docking prediction of protein-protein complexes and symmetric multimers. Bioinformatics. 30, 1771-1773 (2014).
Y. Yan, et al., The HDOCK server for integrated protein-protein docking. Nat. Protoc. 15, 1829-1852 (2020).
A. Tovchigrechko, I. A. Vakser, GRAMM-X public web server for protein-protein docking. Nucleic Acids Res. 34, W310-4 (2006).
M. Torchala, et al., SwarmDock: a server for flexible protein-protein docking. Bioinformatics. 29, 807-809 (2013).
D. Schneidman-Duhovny, et al., PatchDock and SymmDock: servers for rigid and symmetric docking. Nucleic Acids Res. 33, W363-7 (2005).
G. Q. Dong, H. Fan, D. Schneidman-Duhovny, B. Webb, A. Sali, Optimized atomic statistical potentials: assessment of protein interfaces and loops. Bioinformatics. 29, 3158-3166 (2013).
J. Armstrong, et al., Progressive alignment with Cactus: a multiple-genome aligner for the thousand-genome era (2019), p. 730531.
B. Paten, et al., Cactus: Algorithms for genome multiple sequence alignment. Genome Res. 21, 1512-1528 (2011).
M. D. Smith, et al., Less Is More: An Adaptive Branch-Site Random Effects Model for Efficient Detection of Episodic Diversifying Selection. Mol. Biol. Evol. 32, 1342-1353 (2015).
S. L. K. Pond, et al., HyPhy: hypothesis testing using phylogenies. Bioinformatics. 21, 676-679 (2004).
K. S. Pollard, et al., Detection of nonneutral substitution rates on mammalian phylogenies. Genome Res. 20, 110-121 (2010).
M. J. Hubisz, K. S. Pollard, A. Siepel, PHAST and RPHAST: phylogenetic analysis with space/time models. Brief Bioinform. 12, 41-51 (2011).
R. Ramani, et al., PhastWeb: a web interface for evolutionary conservation scoring of multiple sequence alignments using phastCons and phyloP. Bioinformatics. 35, 2320-2322 (2019).
W. A. Ray, Evaluating medication effects outside of clinical trials: new-user designs. Am. J. Epidemiol. 158, 915-920 (2003).
S. Schneeweiss, A basic study design for expedited safety signal evaluation based on electronic healthcare data. Pharmacoepidemiol. Drug Saf 19, 858-868 (2010).
H. Quan, et al., Coding algorithms for defining comorbidities in ICD-9-CM and ICD-10 administrative data. Med. Care. 43, 1130-1139 (2005).
P. C. Austin, Balance diagnostics for comparing the distribution of baseline covariates between treatment groups in propensity-score matched samples. Stat. Med. 28, 3083-3107 (2009).
WHO R&D Blueprint novel Coronavirus: COVID-19 Therapeutic Trial Synopsis.
World Health Organization, 2020.
J. Li, X. Qian, J. Hu, B. Sha, Crystal structure of Tom71 complexed with Hsp82 C-terminal fragment (2009), doi: 10.2210/pdb3fp2/pdb.

1. A method of identifying an interaction between a pathogen protein and a host protein, the method comprising:
(a) identifying a first pathogen protein that co-localizes with a first host protein in one or a plurality of bioassays;
(b) calculating a differential interaction score (DIS) corresponding to a pathogen protein and a host protein in a sample; and
(c) correlating the DIS with the likelihood that the dysfunctional protein-protein interaction is a causal agent of pathogenicity of the pathogen.

2. (canceled)

3. The method of claim 1, wherein the bioassay comprises one or a combination of: mass spectrometry analysis is performed on a plurality of samples from a population of subjects infected with the pathogen; siRNA knockdown analysis, CRISPR-mediated knockout analysis, infectivity analysis; and co-immunoprecipitation.

4. The method of claim 1 further comprising the step of: compiling genetic data about a population of subjects that comprise a mutation in a nucleic acid sequence that encodes the first host protein.

5. The method of claim 1, wherein the one or plurality of bioassays comprises performing a mass spectrometry analysis on a sample associated with the disorder to identify dysfunctional protein-protein interactions associated with the disorder

6. The method of claim 1, wherein each sample comprises a mixture of population of cells unaffected by the disorder and a population of cells expressing a mutation.

7. The method of claim 6, wherein the calculating comprises calculating one or more of a SAINTexpress algorithm score, a CompPASS algorithm score, and a MiST algorithm score.

8. The method of claim 7, wherein the calculating comprises calculating a SAINTexpress algorithm score and a MiST algorithm score.

9. The method of claim 7, wherein the SAINTexpress algorithm score is calculated by a formula:
P(Xᵢⱼ|♦)=π_{T}P(Xᵢⱼ|λᵢⱼ)+(1−π_{T})P(Xᵢⱼ|κᵢⱼ) (1)
wherein Xᵢⱼ is the spectral count for a prey protein i identified in a purification of bait j;
wherein λᵢⱼ is the mean count from a Poisson distribution representing true interaction;
wherein κᵢⱼ is the mean count from a Poisson distribution representing false interaction;
wherein π_{T} is the proportion of true interactions in the data; and
wherein dot notation represents all relevant model parameters estimated from the data for the pair of prey i and bait j.

10. The method of claim 7, wherein the MiST algorithm score is calculated by a first formula:
$${A}_{b , i} = \frac{{\sum }_{r = 1}^{N \text{?}} {Q}_{b , i , r}}{{N}_{R}}$$
$$\text{?} \text{indicates text missing or illegible when filed}$$
wherein A_{b,i} is the abundance of a given bait-prey pair i,b;
wherein Q_{b,i,r} is the quantity of bait-prey pair b,I in a replica r; and
Nᵣ is the number of replicas;
a second formula:
$${R}_{b , i} + \frac{{\sum }_{r = 1}^{N \text{?}} {Q}_{b , i , r} \cdot log ( {Q}_{b , i , r} )}{{{log}_{2} ( {N}_{R} )}^{- 1}}$$
$$\text{?} \text{indicates text missing or illegible when filed}$$
wherein R_{b,i} is the reproducibility of a given bait-prey pair b,I; and
a third formula:
$${S}_{b , i} = \frac{{A}_{b , i}}{{\sum }_{b = 1}^{N \text{?}} {A}_{b , i}}$$
$$\text{?} \text{indicates text missing or illegible when filed}$$
wherein S_{b,i} is the specificity of a given bait-prey pair b,i; and
wherein N_{B} is the number of baits.

11. The method of claim 7, wherein the CompPASS algorithm score is calculated by a Z-score formula pair:
$$\begin{matrix}{\overline{x}}_{j} = \frac{{\sum }_{\text{?}}^{\text{?}} {x}_{i , j}}{k} & \text{?} n = 1 , 2 , … m\end{matrix} \qquad ( Eq . 1 )$$
$${z}_{i , j} = \frac{{x}_{i , j} - {\overline{x}}_{j}}{{σ}_{j}} \qquad ( Eq . 2 )$$
$$\text{?} \text{indicates text missing or illegible when filed}$$
wherein X is the TSC;
wherein i is the bait number;
wherein j is the interactor;
wherein n is which interactor is being considered;
wherein k is the total number of baits; and
wherein s is the standard deviation of the TSC mean;
a S-score formula:
$$\begin{matrix}{S}_{i , j} = \sqrt{( \frac{k}{{\sum }_{\text{?}}^{\text{?}} \text{?}} ) {x}_{i , j}} ; & {f}_{i , j} = \{ \begin{matrix}1 \text{?} {x}_{i , j} > 0 \\ {x}_{i , j}\end{matrix}\end{matrix} \qquad ( Eq . 3 )$$
$$\text{?} \text{indicates text missing or illegible when filed}$$
wherein f is 0 or 1;
a D-score formula:
$$\begin{matrix}{D}_{\text{?}} = \sqrt{{( \frac{k}{{\sum }_{\text{?}}^{\text{?}} \text{?}} )}^{P} {x}_{i , j}} ; & \begin{matrix}{f}_{i , j} = \{ \begin{matrix}1 : {x}_{i , j} > 0 \\ {x}_{i , j}\end{matrix} \\ p = \begin{matrix}number of \text{?} in \\ which the \text{?} is present\end{matrix}\end{matrix}\end{matrix} \qquad ( Eq . 4 )$$
$$\text{?} \text{indicates text missing or illegible when filed}$$
wherein p is 1 or 2; and
a WD-score formula:
$${WD}_{i , j} = \sqrt{{( \frac{k}{{\sum }_{\text{?}}^{\text{?}} \text{?}} {ω}_{j} )}^{P} {x}_{i , j}} \qquad ( Eq . 5 )$$
$${ω}_{j} = ( \frac{{σ}_{j}}{{\overline{x}}_{j}} ) , \qquad \begin{matrix}{\overline{x}}_{j} = \frac{{\sum }_{\text{?}}^{\text{?}} {x}_{i , j}}{k} & \text{?} n = 1 , 2 , … m ,\end{matrix} \qquad \begin{matrix}if {ω}_{j} \text{?} 1 \text{?} {ω}_{j} = 1 \\ if {ω}_{j} \text{?} 1 \text{?} {ω}_{j} = {ω}_{j}\end{matrix}$$
$$\begin{matrix}{f}_{i , j} = \{ \begin{matrix}1 ; {x}_{i , j} > 0 \\ {x}_{i , j}\end{matrix} & p = \begin{matrix}number of replicates \text{?} in \\ which the interactor is present\end{matrix}\end{matrix}$$
$$\text{?} \text{indicates text missing or illegible when filed}$$
wherein wⱼ is a weight factor
wherein σⱼ is a standard deviation.

12. The method of claim 1, wherein the DIS is calculated by a first formula:
DIS_{A}(b,p)=S_{C1}(b,p)×S_{C2}(b,p)×[1−S_{C3}(b,p)]
wherein DIS_{A}(b,p) is the DIS for each protein-protein interaction (PPI) (b, p) that is conserved in a first bioassay and a second bioassay, but not shared by a third bioassay;
wherein S_{C1}(b,p) is the probability of a PPI being present in the first bioassay;
wherein S_{C2}(b,p) is the probability of a PPI being present in the second bioassay; and
wherein S_{c□}(b,p) is the probability of a PPI being present in the third bioassay; and a second formula:
DIS_{B}(b,p)=[1−S_{C1}(b,p)]×[1−S_{C2}(b,p)]×S_{C3}(b,p
wherein DIS_{B}(b,p) is the DIS score for each PPI (b, p) that is conserved in the third bioassay, but not shared by the first bioassay and the second bioassay;
wherein a (+) sign is assigned if DIS_{A}(b,p)>DIS_{B}(b,p); and
wherein a (−) sign is assigned if DIS_{A}(b,p)<DIS_{B}(b,p).

13-25. (canceled)

26. A method of identifying an interaction between a first protein and a second protein, wherein the first protein is associated with a disorder of a subject, the method comprising:
(a) identifying a first protein that co-localizes with the second protein in one or a plurality of bioassays;
(b) calculating a differential interaction score (DIS) corresponding to the first protein and a second protein in a sample from the subject; and
(c) correlating the DIS with the likelihood that the dysfunctional protein-protein interaction is a causal agent of pathogenicity of the pathogen.

27. The method of claim 26, wherein the sample is a population of cells.

28. The method of claim 26, wherein the bioassay comprises one or a combination of: mass spectrometry analysis is performed on a plurality of samples from a population of subjects infected with the pathogen; siRNA knockdown analysis, CRISPR-mediated knockout analysis, infectivity analysis; and co-immunoprecipitation.

29. The method of any of claim 26 further comprising the step of: compiling genetic data about a population of subjects that comprise a mutation in a nucleic acid sequence that encodes the first protein.

30. The method of, claim 26 wherein the one or plurality of bioassays comprises performing a mass spectrometry analysis on a sample associated with the disorder to identify dysfunctional protein-protein interactions associated with the disorder.

31.-50. (canceled)

51. A method of identifying a subject likely to respond to a disorder treatment, the method comprising:
a. calculating a differential interaction score (DIS); and
b. correlating the DIS with a likelihood that a dysfunctional protein-protein interaction is a causal agent of the disorder,
wherein if the DIS score is above a first threshold, then the subject is likely to respond to a disorder treatment based upon the causal agent, and
wherein if the DIS score is below the first threshold, then the subject is not likely to respond to the disorder treatment based upon the causal agent.

52. The method of claim 51, further comprising:
a. compiling genetic data about a population of subjects comprising the subject, wherein the population of subjects has a mutation candidate that causes the disorder; and
b. performing a mass spectrometry analysis on a sample associated with the disorder to identify dysfunctional protein-protein interactions associated with the disorder.

53. A method of predicting a likelihood that a subject does or does not respond to a disorder treatment, the method comprising:
a. compiling genetic data about a population of subjects that has a mutation candidate that causes a disorder, wherein the population of subjects includes the subject;
b. performing a mass spectrometry analysis on a sample associated with the disorder to identify dysfunctional protein-protein interactions associated with the disorder;
c. calculating a differential interaction score (DIS);
d. correlating the DIS with the likelihood that the dysfunctional protein-protein interaction is the causal agent of the disorder; and
e. selecting a treatment for the subject based upon the causal agent.

54. The method of claim 53, further comprising:
(f) comparing the DIS score to a first threshold; and
(g) classifying the subject as being likely to respond to a disorder treatment,
wherein each of steps (f) and (g) are performed after step (c), and
wherein the first threshold is calculated relative to a first control dataset.

55. The method of claim 54, wherein the disorder is a viral infection.

56. The method of claim 55, wherein the viral infection is due to a Coronavirus.

57. A computer program product encoded on a computer-readable storage medium, wherein the computer program product comprises instructions for:
a. identifying protein-protein interactions associated with the disorder; and
b. calculating a differential interaction score (DIS).

58. The computer program product of claim 57, further comprising a step of correlating the DIS with the likelihood that the dysfunctional protein-protein interaction is a causal agent of the disorder.

59. The computer program product of claim 57, further comprising instructions for selecting a treatment for the subject based upon the causal agent.

60. The computer program product of claim 57, further comprising instructions for:
(d) comparing the DIS score to a first threshold; and
(e) classifying the subject as being likely to respond to a disorder treatment,
wherein each of steps (d) and (e) are performed after step (c), and
wherein the first threshold is calculated relative to a first control dataset.

61. A system comprising the computer program product of claim 57, and one or more of:
a. a processor operable to execute programs; and
b. a memory associated with the processor.

62-66. (canceled)

67. A method of selecting a disorder treatment for a subject in need thereof, the method comprising:
a. identifying genetic data from the subject in need of treatment;
b. comparing the genetic data from the subject to a compilation of genetic data from population of subjects that has a mutation candidate that causes a disorder, wherein the population of subjects includes the subject in need thereof;
c. performing a mass spectrometry analysis on a sample from the subject associated with the disorder to identify dysfunctional protein-protein interactions associated with the disorder;
d. calculating a differential interaction score (DIS);
e. correlating the DIS with the likelihood that the dysfunctional protein-protein interaction is a causal agent of the disorder; and
f. selecting a disorder treatment for the subject based upon the causal agent.

68. The method of claim 0, wherein the step of identifying the genetic information from a subject comprises sequencing the genetic information from a biopsy or sample obtained from the subject.

69. The method of claim 0, wherein the calculating of the DIS score is calculated by a first formula:
DIS_{A}(b,p)=S_{C1}(b,p)×S_{C2}(b,p)×[1−S_{C3}(b,p)]
wherein DIS_{A}(b,p) is the DIS for each PPI (b, p) that is conserved in a first cell line and a second cell line, but not shared by a third cell line;
wherein S_{C1}(b,p) is the probability of a PPI being present in the first cell line;
wherein S_{C2}(b,p) is the probability of a PPI being present in the second cell line; and
wherein S_{c□}(b,p) is the probability of a PPI being present in the third cell line; and a second formula:
DIS_{B}(b,p)=[1−S_{C1}(b,p)]×[1−S_{C2}(b,p)]×S_{C3}(b,p
wherein DIS_{B}(b,p) is the DIS score for each PPI (b, p) that is conserved in the third cell line, but not shared by the first cell line and the second cell line;
wherein a (+) sign is assigned if DIS_{A}(b,p)>DIS_{B}(b,p); and
wherein a (−) sign is assigned if DIS_{A}(b,p)<DIS_{B}(b,p).

70-74. (canceled)

75. A method of constructing a three-dimensional (3D) structure of a protein comprising:
a. obtaining a molecular 3D structure of the protein using one or a plurality of structural-biology techniques;
b. obtaining a predicted 3D structure of the protein based on sequence using one or a plurality of deep neural networks;
c. dividing the predicted 3D structure into a plurality of overlapping regions;
d. rigid-body fitting the plurality of overlapping regions against the molecular 3D structure;
e. examining a plurality of regions with top scoring fits and generating new region boundaries;
f. combining the plurality of regions with top scoring fits into a complete 3D protein structure; and
g. refining the complete 3D protein structure into the molecular 3D structure to construct the 3D structure of the protein.

76. The method of claim 75, further comprising repeating steps d) and e) for one or a plurality of times.

77. The method of claim 75, wherein the one or plurality of structural-biology techniques are chosen from cryogenic electron microscopy (cryo-EM), cryo-electron tomography (cryo-ET), nuclear magnetic resonance (NMR) spectroscopy, X-ray crystallography, and small-angle X-ray scattering (SAXS).

78. The method of claim 75, wherein the molecular 3D structure of the protein is obtained using cryo-EM.

79. The method of claim 75, wherein the molecular 3D structure of the protein has a resolution of about 20 ångströms (□) or better.

80-84. (canceled)
